(19) United States
(12) Patent Application Publication
(10) Pub. No.: US 2013/0217592 A1
(43) Pub. Date: Aug. 22, 2013

(54) ARRAYS AND METHODS COMPRISING M. SMITHII GENE PRODUCTS

(71) Applicant: The Washington University, (US)

(72) Inventors: Buck Sparrow Samuel, St. Louis, MO (US); Elizabeth E. Hansen, St. Louis, MO (US); Jeffrey I. Gordon, St. Louis, MO (US)

(73) Assignee: THE WASHINGTON UNIVERSITY, St. Louis, MO (US)

(21) Appl. No.: 13/764,427

(22) Filed: Feb. 11, 2013

Related U.S. Application Data
(63) Continuation-in-part of application No. 12/627,961, filed on Nov. 30, 2009, now abandoned.
Application No. 12/627,961 is a continuation-in-part of application No. PCT/US2008/065344, filed on May 30, 2008.
(60) Provisional application No. 60/932,457, filed on May 31, 2007.

(51) Int. Cl. C12N 15/10

(52) U.S. Cl. CPC C12N 15/1086
USPC 506/10; 506/17; 506/18

(57) ABSTRACT

The present invention encompasses arrays and methods related to the genome of M. smithii.

### CROSS REFERENCE TO RELATED APPLICATIONS

This application is a continuation-in-part of U.S. application Ser. No. 12/627,961, filed on Nov. 30, 2008, which is a continuation-in-part of application No. PCT/US2008/065344, filed on May 30, 2008, which claims the priority of U.S. provisional application No. 60/932,457, filed on May 31, 2007, each of which is hereby incorporated by reference in its entirety.

### GOVERNMENTAL RIGHTS

This invention was made with government support under Grant numbers DK30292 and DK70077 awarded by the National Institutes of Health. The government has certain rights in the invention.

### FIELD OF THE INVENTION

The present invention encompasses arrays and methods related to the genome of M. smithii.

### BACKGROUND OF THE INVENTION

### I. Weight Problems and Current Approaches

According to the Center for Disease Control (CDC), over sixty percent of the United States population is overweight, and almost twenty percent are obese. This translates into 38.8 million adults in the United States with a Body Mass Index (BMI) of 30 or above. Obesity is also a world-wide health problem with an estimated 500 million overweight adult humans [body mass index (BMI) of 25.0-29.9 kg/m²] and 250 million obese adults. This epidemic of obesity is leading to worldwide increases in the prevalence of obesity-related disorders, such as diabetes, hypertension, as well as cardiac pathology, and non-alcoholic fatty liver disease (NAFLD).

According to the National Institute of Diabetes, Digestive and Kidney Diseases (NIDDK) approximately 280,000 deaths annually are directly related to obesity. The NIDDK further estimated that the direct cost of healthcare in the U.S. associated with obesity is $51 billion. In addition, Americans spend $33 billion per year on weight loss products. In spite of this economic cost and consumer commitment, the prevalence of obesity continues to rise at alarming rates. From 1991 to 2000, obesity in the U.S. grew by 61%.

Additionally, malnourishment or disease may lead to individuals being under weight. The World Health Organization estimates that one-third of the world is under-fed and one-third is starving. Over 4 million will die this year from malnourishment. One in twelve people worldwide is malnourished, including 160 million children under the age of 5.

### II. Gastrointestinal Microbiota

Humans are host to a diverse and dynamic population of microbial symbionts, with the majority residing within the distal intestine. The gut microbiota contains representatives from ten known divisions of the domain Bacteria, with an estimated 500-1000 species-level phylogenetic types present in a given healthy adult human; the microbiota is dominated by members of two divisions of Bacteria, the Bacteroidetes and the Firmicutes. Members of the domain Archaea are also represented, most prominently by a methanogenic Euryarchaeote, Methanobrevibacter smithii and occasionally Methanosphaera stadtmanae. The density of colonization increases by eight orders of magnitude from the proximal small intestine (10³) to the colon (10¹¹). The distal intestine is an anoxic bioreactor whose microbial constituents help the subject by providing a number of key functions: e.g., breakdown of otherwise indigestible plant polysaccharides and regulating subject storage of the extracted energy; biotransformation of conjugated bile acids and xenobiotics; degradation of dietary oxalates; synthesis of essential vitamins; and education of the immune system.

Dietary fiber is a key source of nutrients for the microbiota. Monosaccharides are absorbed in the proximal intestine, leaving dietary fiber that has escaped digestion (e.g. resistant starches, fructans, cellulose, hemicelluloses, pectins) as the primary carbon sources for microbial members of the distal gut. Fermentation of these polysaccharides yields short-chain fatty acids (SCFAs; mainly acetate, butyrate and propionate) and gases (H₂ and CO₂). These end products benefit humans. For example, SCFAs are an important source of energy, as they are readily absorbed from the gut lumen and are subsequently metabolized in the colonic mucosa, liver, and a variety of peripheral tissues (e.g., muscle). SCFAs also stimulate colonic blood flow and the uptake of electrolytes and water.

### III. Methanogens

Methanogens are members of the domain Archaea. Methanogens thrive in many anaerobic environments together with fermentative bacteria. These habitats include natural wetlands as well as man-made environments, such as sewage digesters, landfills, and bioreactors. Hydrogen-consuming, mesophilic methanogens are also present in the intestinal tracts of many invertebrate and vertebrate species, including termites, birds, cows, and humans. Using methane breath tests, clinical studies estimate that between 50 and 80 percent of humans harbor methanogens.

Culture- and non-culture-based enumeration studies have demonstrated that members of the Methanobrevibacter genus are prominent gut mesophilic methanogens. The most comprehensive enumeration of the adult human colonic microbiota reported to date found a single predominant archaeal species, Methanobrevibacter smithii. This gram-positive-staining Euryarchaeote can comprise up to 10¹⁰ cells/g feces in healthy humans, or ˜10% of all anaerobes in the colons of healthy adults.

A focused set of nutrients are consumed for energy by methanogens: primarily H₂/CO₂, formate, acetate, but also methanol, ethanol, methylated sulfur compounds, methylated amines and pyruvate. These compounds are typically converted to CO₂ and methane (e.g. acetate) or reduced with H₂ to methane alone (e.g. methanol or CO₂). Some methanogens are restricted to utilizing only H₂/CO₂ (e.g. Methanobrevibacter arbophilicus), or methanol (e.g. Methanospaera stadtmanae). Other more ubiquitous methanogens exhibit greater metabolic diversity, like Methanosarcina species. In vitro studies suggest that M. smithii is intermediate in this metabolic spectrum, consuming H₂/CO₂ and formate as energy sources.

### IV. Anaerobic Microbial Fermentation in the Mammalian Intestine

Fermentation of dietary fiber is accomplished by syntrophic interactions between microbes linked in a metabolic food web, and is a major energy-producing pathway for members of the Bacteroidetes and the Firmicutes. Bacteroides thetaiotaomicron has previously been used as a model bacterial symbiont for a variety of reasons: (i) it effectively ferments a range of otherwise indigestible plant polysaccharides in the human colon; (ii) it is genetically manipulatable; and, (iii) it is a predominant member of the human distal intestinal microbiota. Its 6.26 Mb genome has been sequenced: the results reveal that B. thetaiotaomicron has the largest collection of known or predicted glycoside hydrolases of any prokaryote sequenced to date (226 in total; by comparison, our human genome only encodes 98 known or predicted glycoside hydrolases). B. thetaiotaomicron also has a significant expansion of outer membrane polysaccharide binding and importing proteins (over 200 paralogs of two starch binding proteins known as SusC and SusD), as well as a large repertoire of environmental sensing proteins [e.g. 50 extra-cytoplasmic function (ECF)-type sigma factors; 25 anti-sigma factors, and 32 novel hybrid two-component systems]. Functional genomics studies of B. thetaiotaomicron in vitro and in the ceca of gnotobiotic mice, indicates that it is capable of very flexible foraging for dietary (and host-derived) polysaccharides, allowing this organism to have a broad niche and contributing to the functional stability of the microbiota in the face of changes in the diet.

In vitro biochemical studies of B. thetaiotaomicron and closely related Bacteroides species (B. fragilis and B. succinogenes) indicate that their major end products of fermentation are acetate, succinate, H₂ and CO₂. Small amounts of pyruvate, formate, lactate and propionate are also formed.

### V. Removal of Hydrogen from the Intestinal Ecosystem is Important for Efficient Microbial Fermentation

Anaerobic fermentation of sugars causes flux through glycolytic pathways, leading to accumulation of NADH (via glyceraldehyde-3P dehydrogenase) and the reduced form of ferredoxin (via pyruvate:ferredoxin oxidoreductase). B. thetaiotaomicron is able to couple NAD⁺ recovery to reduction of pyruvate to succinate (via malate dehydrogenase and fumarase reductase), or lactate (via lactate dehydrogenase). Oxidation of reduced ferredoxin is easily coupled to production of H₂. However, H₂ formation is, in principle, not energetically feasible at high partial pressures of the gas. In other words, lower partial pressures of H₂ (1-10 Pa) allow for more complete oxidation of carbohydrate substrates. The subject removes some hydrogen from the colon by excretion of the gas in the breath and as flatus. However, the primary mechanism for eliminating hydrogen is by interspecies transfer from bacteria by hydrogenotrophic methanogens. Formate and acetate can also be transferred between some species, but their transfer is complicated by their limited diffusion across the lipophilic membranes of the producer and consumer. In areas of high microbial density or aggregation like in the gut, interspecies transfer of hydrogen, formate and acetate is likely to increase with decreasing physical distance between microbes.

Methanogen-mediated removal of hydrogen can have a profound impact on bacterial metabolism. Not only does re-oxidation of NADH occur, but end products of fermentation undergo a shift from a mixture of acetate, formate, H₂, CO₂, succinate and other organic acids to predominantly acetate and methane with small amounts of succinate. This facilitates disposal of reducing equivalents, and produces a potential gain in ATP production due to increased acetate levels. For example, a reduction in hydrogen allows Clostridium butyricum to acquire 0.7 more ATP equivalents from fermentation of hexose sugars. Co-culture of M. smithii with a prominent cellulolytic ruminal bacterial species, Fibrobacter succinogenes S85, results in augmented fermentation, as manifested by increases in the rate of ATP production and organic acid concentrations. Co-culture of M. smithii association with Ruminococcus albus eliminates NADH-dependent ethanol production from acetyl-CoA, thereby skewing bacterial metabolism towards production of acetate, which is more energy yielding. H₂-producing fibrolytic bacterial strains from the human colon exhibit distinct cellulose degradation phenotypes when co-cultured with M. smithii, indicating that some bacteria are more responsive to syntrophy with methanogens.

While there is suggestive evidence that methanogens cooperate metabolically with members of Bacteroides, studies have not elucidated the impact of this relationship on a subject's energy storage or on the specificity and efficiency of carbohydrate metabolism. Colonization of adult germ-free mice with M. smithii and/or B. thetaiotaomicron revealed that the methanogen increased the efficiency and changed the specificity of bacterial digestion of dietary glycans. Moreover, co-colonized mice exhibited a significantly greater increase in adiposity compared with mice colonized with either organism alone.

### SUMMARY OF THE INVENTION

One aspect of the present invention encompasses an array. The array comprises a substrate having disposed thereon at least one nucleic acid, wherein the nucleic acid comprises a nucleic acid sequence selected from the nucleic acid sequences listed in Table A.

Another aspect of the present invention encompasses an array. The array comprises a substrate having disposed thereon at least one polypeptide, wherein the polypeptide is encoded by a nucleic acid sequence selected from the nucleic acid sequences listed in Table A.

Yet another aspect of the present invention encompasses an array. The array comprises a substrate having diposed thereon at least one nucleic add encoding an adhesin-like protein, wherein the nucleic acid comprises a nucleic acid sequence selected from group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, and 95.

Yet another aspect of the present invention encompasses an array. The array comprises a substrate having diposed thereon at least one nucleic acid encoding an adhesin-like protein, wherein the nucleic acid comprises a nucleic acid sequence selected from group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, and 95. In addition, the array further comprises at least one nucleic acid sequence selected from the group consisting of SEQ ID NOs: 97-2140

Yet another aspect of the present invention encompasses an array. The array comprises a substrate having disposed thereon at least one polypeptide, wherein the polypeptide is encoded by a nucleic acid sequence selected from group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, and 95.

Yet another aspect of the present invention encompasses an array. The array comprises a substrated having disposed thereon at least one polypeptide, wherein the polypeptide comprises at least one amino acid sequence selected from the group consisting of SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, and 96.

Yet another aspect of the present invention encompasses an array. The array comprises a substrate having disposed thereon at least one polypeptide, wherein the polypeptide is encoded by a nucleic acid sequence selected from group consisting of SEQ ID NOs: 97-2140.

Yet another aspect of the present invention encompaases a method of selecting a compound that has efficacy for modulating a gene product of M. smithii present in the gastrointestinal tract of a subject, wherein the gene product correlates with a biomolecule selected from the group consisting of SEQ ID NOs: 1-96. The method comprises comparing a plurality of biomolecules from M. smithii before and after administration of a compound for modulating a gene product of M. smithii, such that if the abundance of a biomolecule that correlates with the gene product is modulated, the compound is efficacious in modulating a gene product of M. smithii, and selecting a compound that modulates a M. smithii gene product.

Yet another aspect of the present invention encompasses a method of selecting a compound that has efficacy for modulating a gene product of M. smithii present in the gastrointestinal tract of a subject. The method comprises comparing an M. smithii gene profile to a gene profile of the subject, identifying a gene product of the M. smithii gene profile that is divergent from a corresponding gene product of the subject gene profile, or absent in the gene profile of the subject, and selecing a compound that modulates the M. smithii gene product but does not substantially modulate the corresponding divergent gene product of the subject.

Still another aspect of the invention encompasses a method for modulating a gene product of M. smithii present in the gastrointestinal tract of a subject. The method comprises administering to the subject an HMG-CoA reductase inhibitor. The inhibitor may be formulated for release in the distal portion of the subject's gastrointestinal tract and thereby substantial inhibit more of the HMG-CoA reductase of M. smithii compared to the subject's HMG-CoA reductase.

Other aspects and iterations of the invention are described more thoroughly below.

### DESCRIPTION OF THE DRAWINGS

FIG. 1. depicts a micrograph and a graph illustrating that M. smithii produces glycans that mimic those produced by humans—(A) TEM of M. smithii harvested from the ceca of adult GF mice after a 14 day colonization. The inset shows a comparable study of stationary phase M. smithii recovered from a batch fermentor containing Methanobrevibacter complex medium (MBC). Note that the size of the capsule is greater in cells recovered from the cecum (open vs. closed arrow). (B) Comparison of glycosyltransferase (GT), glycosylhydrolase (GH) and carbohydrate esterase (CE) families (defined in CAZy; Table 10) represented in the genomes of the following sequenced methanogens (see Table 5): Msm, Methanobrevibacter smithii; Msp, Methanosphaera stadtmanae; Mth, Methanothermobacter thermoautotrophicus; Mac, Methanosarcina acetivorans; Mba, M. barkeri; Mma, M. mazei; Mmp, Methanococcus maripaludis; Mja, M. jannaschii; Mhu, Methanospirillum hungatei; Mbu, Methanococcoides burtonii; and Mka, Methanopyrus kandleri. Gut methanogens (highlighted in orange) have no GH or CE family members, but have a larger proportion of family 2 GTs (^{ψ}, p<0.00005 based on binomial test for enrichment vs. non-gut associated methanogens). Scale bar, 100 μm in panel A.

FIG. 2. depicts graphs and diagrams illustrating functional genomic and biochemical assays of M. smithii metabolism in the ceca of gnotobiotic mice. (A) In silico metabolic reconstructions of M. smithii pathways involved in (i) methanogenesis from formate, H₂/CO₂, and alcohols, (ii) carbon assimilation from acetate and bicarbonate, and (iii) nitrogen assimilation from ammonium. Abbreviations: Acs, acetyl-CoA synthase; Adh, alcohol dehydrogenase; Ags, 18 α-ketoglutarate synthase; AmtB, ammonium transporter; BtcA/B, bicarbonate (HCO₃) ABC transporter; Cab, carbonic anhydrase; CH₃, methyl; CoA, coenzyme A; CoB, coenzyme B; CoM, coenzyme M; COR, corrinoid; F₄₂₀, cofactor F₄₂₀; F₄₃₀, cofactor F₄₃₀; Fd, ferredoxin (ox-oxidized, red-reduced); FdhAB, formate dehydrogenase subunits; FdhC, formate transporter; Fno, F₄₂₀-dependent NADP reductase; Ftr, formylmethanofuran:tetrahydromethanopterin (H₄MPT) formyltransferase; Fum, fumarate hydratase; Fwd, tungsten formylmethanofuran dehydrogenase; GdhA, glutamate dehydrogenase; GInA, glutamine synthetase; GltA/B, glutamate synthase subunits A and B; Hmd, H₂-forming methylene-H₄MPT dehydrogenase; Kor, 2-oxoglutarate synthase; Mch, methenyl-H₄MPT cyclohydrolase; Mcr, methyl-CoM reductase; Mdh, malate dehydrogenase; MeOH, methanol; Mer, methylene-H₄MPT reductase; MFN, methanofuran; MtaB, methanol:cobalamin methyltransferase; Mtd, F₄₂₀-dependent methylene-H₄MPT dehydrogenase; Mtr, methyl-H₄MPT:CoM methyltransferase; NH4, ammonium; OA, oxaloacetate; PEP, phosphoenol pyruvate; Por, pyruvate:ferredoxin oxidoreductase; Pps, phosphoenolpyruvate synthase; PRPP, 5-phospho-a-D-ribosyl-1-pyrophosphate; Pyc, pyruvate carboxylase; RfaS, ribofuranosylaminobenzene 5′-phosphate (RFA-P) synthase; Sdh, succinate dehydrogenase; Suc, succinyl-CoA synthetase. Potential drug targets are noted (Rx). (B,C,G) qRT-PCR assays of the expression of key M. smithii (Ms) genes in gnotobiotic mice that do or do not harbor B. thetaiotaomicron (Bt)(n=5-6 animals/group; each sample assayed in triplicate; mean values±SEM plotted; see Table 11 for full list of analyses). Results are summarized in Panel A using the following color codes: red, upregulated; green, downregulated; grey, assayed but no significant change; black arrows, transcript not assayed. (D) Ethanol (EtOH) levels in the ceca of mice colonized with B. thetaiotaomicron±M. smithii (n=10-15 animals/group representing 3 independent experiments; each sample assayed in duplicate; mean values±SEM plotted). (E) Ratio of cecal concentrations of glutamine (Gln) and 2-oxoglutarate (2-OG) (n=5 animals/group; samples assayed in duplicate; mean values±SEM). (F) Cecal levels of free Gln (glutamine), Glu (glutamate) and Asn (asparagine) (n=5 animals/group; samples assayed in duplicate; mean values±SEM). (H) Cecal ammonium and urea levels measured in samples used for the assays shown in panels E and F. *, p<0.05; **, p<0.01; ***, p<0.005, according to Student's t-test.

FIG. 3. depicts a diagram of the analysis of the M. smithii pan-genome. Schematic depiction of the conservation of M. smithii PS genes [depicted in the outermost circle where the color code is orange for forward strand ORFs (F) and blue for reverse strand ORFs (R)] in (i) other M. smithii strains (GeneChip-based genotyping of strains Fi, ALI, and B181; circles in increasingly lighter shades of green, respectively), (ii) the fecal microbiomes of two healthy individuals [human gut microbiome (HGM), shown as the red plot in the fifth innermost circle with nucleotide identity plotted from 80% (closest to the purple circle) to 100% (closest to lightest green ring); see also FIG. 9 for details], and (iii) two other members of the Methanobacteriales division, M. stadtmanae (Msp; purple circle), another human gut methanogen, and M. thermoautotrophicus (Mth; yellow circle), an environmental thermophile [mutual best blastp hits (e-value<10⁻²⁰)]. Tick marks in the center of the Figure indicate nucleotide number in kbps. Asterisks denote the positions of ribosomal rRNA operons. Letters highlight distinguishing features among M. smithii genomes: the table below the figure summarizes differences in M. smithii gene content between strains F1, ALI, and B181 as well as the two human fecal metagenomic datasets.

FIG. 4. depicts two illustrations of the analysis of synteny between M. smithii and M. stadtmanae genomes. (A) Dot plot comparison. (B) Results obtained with the Artemis Comparison Tool (Carver et al., (2005) Bioinformatics 21:3422-3) set to tBLASTX and the most stringent confidence level (blue, forward strand; orange, reverse strand). The gut methanogens exhibit limited synteny.

FIG. 5. depicts an illustration of the predicted interaction network of M. smithii clusters of orthologous groups (COGs) based on STRING. Individual M. smithii COGs are represented by nodes (circles; 622 of the 1352 COGs in M. smithii's genome). Predicted interactions are represented by black lines (0.95 confidence interval; summary of 9,765 total predicted interactions are shown). COG conservation among the Methanobacteriales is denoted by node color: red, M. smithii alone; yellow, gut methanogens; green, M. smithii and M. thermoautotrophicus; and gray, all three genomes. Several clusters are highlighted: (A) molybdopterin biosynthesis (methanogenesis from CO₂); (B) ion transport; (C) DNA repair/recombination; (D) antimicrobial transport; (E) sialic acid synthesis; (F) amino acid transport system; (G) HMG-CoA reductase cluster; and (H) conserved archaeal membrane protein cluster. See Table 9 for lists of genes assigned to COGs.

FIG. 6. depicts an illustration, a graph, and a micrograph showing sialic acid production by M. smithii in vitro. (A) M. smithii gene cluster (MSM1535-40) encoding enzymes needed to synthesize sialic acid (N-acetylneuraminic acid; Neu5Ac): CapD, polysaccharide biosynthesis protein/sugar epimerase; DegT, pleiotropic regulatory protein/amidotransferase; NeuS, Neu5Ac cytidylyltransferase; NeuA, CMP-Neu5Ac synthetase; NeuB, Neu5Ac synthase; Gpd, glycerol-3-phosphate dehydrogenase. (B) Reverse phase-HPLC of derivatized M. smithii cell wall extracts. The position of elution of N-acetylneuraminic acid (Neu5Ac) and N-glycolylneuraminic acid (Neu5Gc) standards are shown. The concentration of Neu5Ac species of sialic acid in M. smithii cell walls, when the organism has been cultured in a batch fermentor for 6d in supplemented MBC medium (does not contain any sialic acid sources), is 410 pmol/g wet weight of cells (average of three assays). (C) Lectin staining with fluorescein-labeled SNA (Sambucus nigra agglutinin) shows that M. smithii F1 is decorated with Neu5Ac epitopes (counter stained with DAPI; X100 magnification). The specificity of lectin staining was assessed using E. coli K92 (positive control; sialic acid-producing), B. longum NCC2705 (negative control) and M. smithii cells with no lectin added (background autofluorescence control).

FIG. 7. depicts distinct complements of adhesin-like proteins in gut methanogens (A) A maximum likelihood tree of a CLUSTALW alignment of all adhesin-like proteins (ALPs) in M. smithii (47; red branches) and in M. stadtmanae (38; black branches). Each methanogen possesses specific clades of ALPs. Branches that are supported by bootstrap values >70% are noted. InterPro-based analysis reveals that many of these proteins contain common adhesin domains [i.e., invasin/intimin domains (IPR008964) and pectate lyase folds (IPR011050)]. They also have domains associated with additional functionality (basis for branch highlighting): (i) sugar binding [e.g., galactose-binding-like (IPR008979) and Concanavalin A-like lectin (IPR013320)]; (ii) glycosaminoglycan (GAG)-binding (IPR012333); or (iii) peptidase activity [e.g., carboxypeptidase regulatory region (IPR008969) and beta-lactamase/transpeptidase-like fold (IPR012338)]; (iv) transglycosidase activity [e.g., glycosidase superfamily domains (SSF51445)]; and/or (v) general adhesin/porin activity [e.g., Bacillus anthracis OMP repeats/DUF11 (IPR001434)]. See Table 12 for a complete list of ALPs and domains identified by InterProScan. (B) qRT-PCR analyses of the expression of selected M. smithii ALP genes in the ceca of gnotobiotic mice colonized with M. smithii (Msm) alone or with Msm and B. thetaiotaomicron (Bt) [n=5-6/group; each sample assayed in triplicate; mean values±SEM are plotted]. *, P<0.05; ***, P<0.005.

FIG. 8. depicts an illustration showing the importance of the molybdopterin biosynthesis pathway for methanogenesis from carbon dioxide in M. smithii. (A) In silico metabolic reconstruction of the predicted molybdopterin biosynthesis pathway encoded by the M. smithii genome. Molybdopterin can chelate molybdate (MoO₄⁻) or tungstate (WO₄²⁻) ions. Abbreviations: MoaABCE, molybdenum cofactor biosynthesis proteins A (MSM0849, MSM1406), B (MSM0840), C (MSM1362), and E (MSM0130); MoeAB, molybdopterin biosynthesis proteins A (MSM1343) and B (MSM0729); ModABC, molybdate ABC transport system (MSM1609-11); MobAB, molybdopterin-guanine dinucleotide (MGD) biosynthesis proteins A (MSMO240) and B (MSM1407); PP, pyrophosphate. Note that the molybdate transporter may also be used for WO₄²⁻, as no dedicated complex has been identified for its transport. (B) Schematic of the first step in the methanogenesis pathway from carbon dioxide (CO₂) catalyzed by tungsten-containing formylmethanofuran dehydrogenase (Fwd; MSM1408-14, MSM0783, MSM1396). Essential cofactors for this reaction include tungsten delivered by MGD, methanofuran (MFN), and ferridoxin [Fd; converted from a reduced (red) to oxidized (ox) form during the reaction].

FIG. 9. illustrates the divergence in genes involved in surface variation, genome evolution, and metabolism among M. smithii strains and in the human gut microbiomes of two healthy adults. Each of the 139,521 unidirectional reads in the metagenomic dataset (Gill et al., (2006) Science 312, 1355-9) were compared to the M. smithii PS genome using NUCmer. Reads with nucleotide sequence identity ≧80% (present) are plotted. A summary of representation of M. smithii PS genes present in the metagenomic dataset is displayed at the bottom of the graph (92% of the total ORFs). [Note that the gaps are indications of genome plasticity in the dataset, and include transposases, restriction-modification systems and prophage genes.] Selected regions of heterogeneity (divergence) are highlighted; genes in these regions are involved in the metabolism of bacterial products, recombination/repair machinery (Recomb), anti-microbial resistance (AntiMicrob), surface variation (Surface), and adhesion (ALPs). See Table 2 for details.

FIG. 10 depicts three graphs showing the dose effect of atorvastatin (A), pravastatin (B), and rosuvastatin (C) on M. smithii strain PS.

FIG. 11 depicts three graphs showing the dose effect of atorvastatin (A), pravastatin (B), and rosuvastatin (C) on M. smithii strain F1.

FIG. 12 depicts three graphs showing the dose effect of atorvastatin (A), pravastatin (B), and rosuvastatin (C) on M. smithii strain ALI.

FIG. 13 depicts three graphs showing the dose effect of atorvastatin (A), pravastatin (B), and rosuvastatin (C) on M. smithii strain B181.

FIG. 14 depicts three graphs showing the effect of statins (concentration of 1 mM) on B. thetaiotaomicron.

FIG. 15 depicts two photographs of the PHAT system described in the Examples. Panel A shows the pressurized incubation vessels within the anaerobic chamber, while Panel B shows an individual PHAT system outside of the chamber.

FIG. 16 depicts three graphs showing the correlation of methanogen levels in the fecal microbiota of MZ and DZ co-twins. The presence and levels of fecal methanogens were defined by qPCR assay that targeted the mcrA gene in samples obtained from MZ twin pairs (A) (n=40) and DZ twin pairs (B) (n=28). Dashed lines represent 95% confidence intervals for linear regression. (C) Correlation between mcrA levels in fecal samples collected at two time points per individual (2-mo interval between sampling). All axes in A-C are log₁₀ (genome equivalents per ng total DNA+1).

FIG. 17 depicts a schematic showing ammonia assimilation for M. smithii and charts showing normalized RNA-Seq reads assigned to the gene encoding an ammonium transporter (AmtB) and ECs involved in ammonia assimilation. (A) Overview of the two pathways in M. smithii for assimilating ammonia: The energy-dependent glutamine synthetase-glutamate synthase pathway has high affinity for ammonia (red arrow); an ATP-independent pathway has lower affinity (orange). (B) Strain-specific differences in the relative expression of components of the high affinity Gln pathway and the energy-independent low affinity pathway for ammonia assimilation. Mean values±SEM are plotted. Colors represent components of the two pathways shown in A; color codes are coordinated between A and B. (C) Strain-specific differences in levels of expression of amtB. P<0.0001 by one-way ANOVA.

FIG. 18 depicts graphs showing differential expression of M. smithii adhesin-like proteins (ALPs). Members of selected ALP OGUs with strain-specific differences in their expression profiles (A) and strain-specific, as well as OGU-associated, differences in their sensitivity to levels of formate during midlog phase growth (B). OGUs 112, 412, 827, and 208 exhibit strain-specific differences in their expression irrespective of formate concentration (one-way ANOVA, P<0.0001), whereas OGUs 226, 287, 18, 133, and 37 contain at least one representative that is significantly regulated by formate concentration. Mean values±SEM are plotted (n=6 replicates per condition). * indicates a ≧2-fold difference, PPDE≧0.97.

FIG. 19 depicts schematics showing the phylogentic lineage of bacterial taxa that co-occur with human gut methanogens (M. smithii) and their phylogenetic lineages. Shown in A-C are sections of the Arb parsimony insertion trees for selected co-occurring lineages. Trees contain all OTUs found in >9 samples and their relatives with cultured representatives or with known biological properties for [Firmicutes; Clostridiales; Cluster I; Gut Clone Group] (A); [Proteobacteria; Delta Proteobacteria; Desulfovibrio] (B); and [Firmicutes; Clostridiales; Cluster IV; Sporobacter/Oscillospira] (C). The Desulfovibrio tree (B) has two OTUs, OTU7973 and OTU12216, that were found in fewer than 10 fecal samples. OTU7973 was only present in samples that were mcrA positive (abbreviated “M.+”). In contrast, all samples that contained OTU12216 were mcrA negative (“M.−”). The branches of the tree are colored by the co-occurrence index (CI), which is calculated as the log-fold difference in the average relative abundance in M. smithii-positive versus -negative samples. Red indicates a positive association with M. smithii; blue, negative; purple, neutral. The CI scores are listed after the OTU name (the number following the colon). OTUs with a significantly higher relative abundance in M. smithii-positive versus -negative individuals (ANOVA, P<0.05 with FDR correction) are marked with a star. Internal branches are colored based on the average value for all of the OTUs descending from that node. The branches were colored across a red-blue spectrum by using −1.8 and +1.8 as min/max values. These values were selected to represent the range of CI scores (which were between −1.71 and 1.8). OTUs always or never detected in M. smithii-positive individuals were assigned the maximum and minimum CI score, respectively; a CI could not be calculated for these OTUs because it would require dividing by zero.

FIG. 20 depicts a graph and images showing the comparison of strains based on their SNP content. Draft M. smithii genomes were aligned by using Mauve, and SNPs were identified within localized collinear blocks (LCBs). (A) Pair-wise comparison of shared SNPs among all 20 strains plus the reference type strain (MsmPS). (B) Comparison of percent shared SNPs among M. smithii strains by familial relationship. The statistical analysis consisted of a one-way ANOVA followed by Tukey's post hoc analysis. (C) Principal components analysis of SNP data reveals clustering by individual and by family.

FIG. 21 depicts graphs and images showing a comparison of M. smithii strains based on their gene content. (A) Overview of M. smithii pan-genome as defined by operational gene units (OGUs) with >90% identity by CD-Hit. (B) Pairwise comparisons of strains for the presence of shared OGUs. Boxes are shaded from light gray to black to display the percent of total OGUs that are shared in a given comparison. The colored inset summarizes M. smithii strain nomenclature and relates the nomenclature to the human donor based on family and the zygosity of co-twins. (C) Principal components analysis of the OGU table shown in B. (D) Comparison of percent shared OGUs of M. smithii strains by familial relationship. Mean values±SEM are plotted. The statistical significance of observed differences between groups was determined one-way ANOVA followed by Tukey's post hoc analysis, with red bars indicating P<0.001 and green bars indicating P<0.01.

FIG. 22 depicts a graph and table showing a rarefaction analysis of gene discovery in the M. smithii pan-genome. (A) Rarefaction curve. Light blue and light orange lines indicate 95% confidence limits. (B) OGUs present in strains as shown by the cumulative number of strains containing the OGU. Just over 1,000 OGUs are present in all 10 strains of a family. The MZ family (blue) has a higher number of OGUs present in a greater number of strains (5-10), whereas the DZ family (orange) has more OGUs present in 2-4 strains.

FIG. 23 depicts graphs and table, which discriminate M. smithii strains based on their content of genes encoding COGs and enzymes with assigned enzyme classification (EC) numbers. (A) COG assignments in core versus variable OGUs distributed over the various strains. COG assignments were given to all possible OGUs, both for core genes (i.e., OGUs containing genes from all strains) and variably represented genes (OGUs containing genes from one or more of the strains). The left column shows the distribution of COG categories in the defined “core” component of the M. smithii pan-genome. COG categories represented in each strain are displayed as the percent of all OGUs in that strain that had an assigned COG annotation. Each COG was assigned a color, which is defined in the key in (B). (C, D) Distribution of strains based on their enzyme classification (EC) assignments. ECs were assigned to protein coding genes in each strain by using KEGG. Canonical correspondence analysis was used to determine which ECs contributed to the variation seen between the strains. ECs located furthest from the origin contribute most to the variance of strains. (E) Results of a binomial test for enrichment or depletion of ECs in various strains after normalizing to the number of genes in that strain that could be assigned a KEGG annotation. Strain prefixes are listed across the table. The total number of genes assigned to a KEGG annotation for each strain is listed below each strain prefix. A description of the EC numbers listed in (E) is provided in the table in (F).

FIG. 24 (A) depicts graphically the growth characteristics of M. smithii strains when cultured in modified MBC medium containing either low or high concentrations of formate. All strains were grown under an atmosphere of 80% H₂/20% CO₂ at 30 psi. Gases were replenished every 6 h. Aliquots were taken at the time of repressurization for measurement of optical density (OD) at 600 nm to monitor growth. (B) depicts graphically the normalized RNA-Seq reads assigned to KEGG gene families involved in the methanogenesis pathway. For each EC, expression is displayed as mean percent normalized counts (normalized per million reads and to the length of the gene). (C) shows a diagram of the methanogenesis pathway, The colors assigned to each EC are coordinated with the diagram, in order to indicate the step at which each EC acts.

FIG. 25 depicts two graphs showing gene and dinucleotide atypicality in strain METSMIALI. (A) Threshold for gene atypicality in strain METSMIALI against the whole-genome model. The vertical axis represents the compositional typicality of each gene in the genome of the METSMIALI type strain. Scores along the vertical axis represent the G-statistic [made negative so as to represent gene typicality following the convention of Tsirigos et al. (57)]. A threshold for the significance of atypical genes has been chosen in two ways: either using a rank order threshold (ref. 57; red points) or by naively assuming a normal distribution and applying the Bonferroni corrected G-test (red plus blue points). In this case, the two methods select similar significance thresholds. (B) Dinucleotide atypicality in the METSMIALI genome. The colored trendlines indicate differences between gene dinucleotide composition and the composition of either the whole-genome (black line) or ribosomal proteins (blue lines). Each trendline represents a moving average over a 50-gene window. The gray lines show gene typicality for each gene against the whole genome model. In order for a gene to be scored as transferred, the individual gene typicality must be below the significance threshold (horizontal lines) for both comparison sets. Tracks along the top of the graph represent gene annotations; from top to bottom, core genome members (thin blue line), ribosomal proteins (blue squares), horizontally transferred genes (green circles), ALP genes (red triangles), degenerate prophage (pink bar), and members of the variable genome (thin black line).

FIG. 26 depicts graphically the correlation between M. smithii transcriptional profiles generated from RNA-Seq versus GeneChip analyses. RNA samples were processed and analyzed by both RNA-Seq and by GeneChip. The two platforms yielded highly similar results (Pearson's correlation r² values: 0.86-0.89, P<2e⁻¹⁶).

FIG. 27 depicts an analysis of proghages present in M. smithii strains schematically. Raw 454 titanium sequencing reads from those strains with predicted prophages (Table 23) were mapped onto the M. smithii type strain prophage sequence (coordinates 1705364:1736208) by using Nucmer and plotted with Mummer (53). Axes are from 80 to 100% similarity. The map is divided into two panels (A) and (B) at approximately the midpoint.

### DETAILED DESCRIPTION

The present invention provides arrays and methods utilizing the genome and proteome of the methanogen M. smithii, which is the predominant methanogen present in the human gastrointestinal tract. Modulating the Archea population of the gastrointestinal tract of a subject, of which M. smithii is a major component, modulates the efficiency and selectivity of carbohydrate metabolism. The genome and proteome of M. smithii may be used, according to the methods presented herein, to promote weight loss or weight gain in a subject. In particular, the methods of the present invention may be used to identify compounds that promote weight loss or weight gain in a subject. The method relies on applicants' discovery that certain M. smithii gene products are conserved between M. smithii strains, yet divergent (or absent) from the correlating gene products expressed by the subject's microbiome or genome. This allows the selection of compounds that specifically modulate the M. smithii gene product, while substantially not modulating the subject's gene product.

### I. Arrays

One aspect of the invention encompasses use of biomolecules in an array. As used herein, biomolecule refers to either nucleic acids derived from a M. smithii genome, or polypeptides derived from a M. smithii proteome. A M. smithii genome or proteome may be utilized to construct arrays that may be used for several applications, including discovery of compounds that modulate one or more M. smithii gene products, judging efficacy of existing weight gain or loss regimes, and for the identification of biomarkers involved in weight gain or loss, or a weight gain or loss related disorder.

The array may be comprised of a substrate having disposed thereon at least one biomolecule. Several substrates suitable for the construction of arrays are known in the art. The substrate may be a material that may be modified to contain discrete individual sites appropriate for the attachment or association of the biomolecule and is amenable to at least one detection method. Alternatively, the substrate may be a material that may be modified for the bulk attachment or association of the biomolecule and is amenable to at least one detection method. Non-limiting examples of substrate materials include glass, modified or functionalized glass, plastics (including acrylics, polystyrene and copolymers of styrene and other materials, polypropylene, polyethylene, polybutylene, polyurethanes, TeflonJ, etc.), nylon or nitrocellulose, polysaccharides, nylon, resins, silica or silica-based materials including silicon and modified silicon, carbon, metals, inorganic glasses and plastics. In an exemplary embodiment, the substrates may allow optical detection without appreciably fluorescing.

A substrate may be planar, a substrate may be a well, i.e. a 1534-, 384-, or 96-well plate, or alternatively, a substrate may be a bead. Additionally, the substrate may be the inner surface of a tube for flow-through sample analysis to minimize sample volume. Similarly, the substrate may be flexible, such as a flexible foam, including closed cell foams made of particular plastics. Other suitable substrates are known in the art.

The biomolecule or biomolecules may be attached to the substrate in a wide variety of ways, as will be appreciated by those in the art. The biomolecule may either be synthesized first, with subsequent attachment to the substrate, or may be directly synthesized on the substrate. The substrate and the biomolecule may both be derivatized with chemical functional groups for subsequent attachment of the two. For example, the substrate may be derivatized with a chemical functional group including, but not limited to, amino groups, carboxyl groups, oxo groups or thiol groups. Using these functional groups, the biomolecule may be attached using functional groups on the biomolecule either directly or indirectly using linkers.

The biomolecule may also be attached to the substrate non-covalently. For example, a biotinylated biomolecule can be prepared, which may bind to surfaces covalently coated with streptavidin, resulting in attachment. Alternatively, a biomolecule or biomolecules may be synthesized on the surface using techniques such as photopolymerization and photolithography. Additional methods of attaching biomolecules to arrays and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, each of which is hereby incorporated by reference in its entirety).

In one embodiment, the biomolecule or biomolecules attached to the substrate are located at a spatially defined address of the array. Arrays may comprise from about 1 to about several hundred thousand addresses. In one embodiment, the array may be comprised of less than 10,000 addresses. In another alternative embodiment, the array may be comprised of at least 10,000 addresses. In yet another alternative embodiment, the array may be comprised of less than 5,000 addresses. In still another alternative embodiment, the array may be comprised of at least 5,000 addresses. In a further embodiment, the array may be comprised of less than 500 addresses. In yet a further embodiment, the array may be comprised of at least 500 addresses.

A biomolecule may be represented more than once on a given array. In other words, more than one address of an array may be comprised of the same biomolecule. In some embodiments, two, three, or more than three addresses of the array may be comprised of the same biomolecule. In certain embodiments, the array may comprise control biomolecules and/or control addresses. The controls may be internal controls, positive controls, negative controls, or background controls.

The biomolecule may be a nucleic acid derived from any M. smithii genome. In some embodiments, a biomolecule may be a nucleic acid derived from the M. smithii genome with the GenBank Accession number CP000678, comprising, in part, nucleic acid sequences labeled MSM001 through MSM1795, inclusive. In other embodiments, a biomolecule may be a nucleic acid derived from a M. smithii genome selected from the group consisting of a M. smithii genome with the GenBank Accession number CP000678, AEKU00000000, AELL00000000, AELM00000000, AELN00000000, AELO00000000, AELP00000000, AELQ00000000, AELR00000000, AELS00000000, AELT00000000, AELU00000000, AELV00000000, AELW00000000, AELX00000000, AELY00000000, AELZ00000000, AEMA00000000, AEMB00000000, AEMC00000000, and AEMD00000000. Such nucleic acids may include RNA (including mRNA, tRNA, and rRNA), DNA, and naturally occurring or synthetically created derivatives. A nucleic acid derived from a M. smithii genome is a nucleic acid that comprises at least a portion of a nucleic acid sequence selected from the nucleic acid sequences listed in Table A, Table B, and/or Table D. The nucleic acid may comprise fewer than 10, at least 10, at least 20, at least 30, at least 40, at least 50, at least 60, at least 70, at least 80, at least 90, at least 100, at least 150, at least 200, or more than 200 bases of a nucleic acid sequence selected from the nucleic acid sequences listed in Table A, Table B, and/or Table D. One embodiment of the invention is an array comprising a substrate, the substrate having disposed thereon at least one nucleic acid, wherein the nucleic acid comprises a nucleic acid sequence selected from the nucleic acid sequences listed in Table A. In another embodiment, the nucleic acid consists of a nucleic acid sequence selected from the nucleic acid sequences listed in Table A. In another embodiment, the nucleic acid comprises a nucleic acid sequence selected from the nucleic acid sequences listed in Table D. In other exemplary embodiments, the nucleic acid consists of a nucleic acid sequence selected from the nucleic acid sequences listed in Table D. In some exemplary embodiments, the nucleic acid comprises a nucleic acid sequence selected from the nucleic acid sequences listed in Table B. In other exemplary embodiments, the nucleic acid consists of a nucleic acid sequence selected from the nucleic acid sequences listed in Table B. In still other exemplary embodiments, the nucleic acid comprises a nucleic acid sequence selected from the nucleic acid sequences listed in Table B, and further comprises a nucleic acid sequence selected from the nucleic acid sequences listed in Table D.

In one embodiment, the nucleic acid or nucleic acids may be selected from the group of nucleic acids listed in Table A, B and D that are conserved among M. smithii strains, but divergent from a corresponding nucleic acid of the subject. In this context, a “corresponding nucleic acid” refers to a nucleic acid sequence of the subject, or the subject's micobiome, that has greater than 75% identity to a nucleic acid sequence of Table A, B or D. The term, “divergent,” as used herein, refers to a sequence of Table A, B or D that has less than 99% identity, but greater than 75% identity, with a nucleic acid sequence of the subject, or the subject's microbiome. For instance, in some embodiments, divergent refers to less than or equal to about 98%, 97%, 96%, 95%, 94%, 93%, 92%, 91%, 90%, 89%, 88%, 87%, 86%, 85%, 84%, 83%, 82%, 81%, 80%, 79%, 78%, 77%, or 76%, identity between the nucleic acid sequence of Table A, B or D and the nucleic acid sequence of the subject. Conversely, the term “conserved,” as used herein, refers to a nucleic acid sequence of one M. smithii strain that has greater than about 90% identity to a nucleic acid sequence from another M. smithii strain.

If a subject, or the subject's microbiome, does not comprise a nucleic acid sequence that has greater than 75% identity to a nucleic acid sequence of Table A, B, or D, that nucleic acid sequence of Table A, B, or D is “absent” from the subject. In certain embodiments, the nucleic acid or nucleic acids of the array of the invention are selected from the group comprising nucleic acid sequences that are absent from the subject gut microbiome or genome. For instance, in one embodiment, the nucleic acid may be selected from the group of nucleic acids designated absent or divergent in Table 2. Percent identity may be determined as discussed below.

Alternatively, the nucleic acid or nucleic acids may be selected from the group of nucleic acids listed in Table A, B and D that are not conserved among M. smithii strains, For example, while the genome of a M. smithii strain may comprise at least one nucleic acid that enodes an adhesin-like protein (ALP), the nucleic acid encoding a particular ALP may not be present in all strains. Stated another way, a nucleic acid encoding a particular type of protein (e.g. an ALP) may show strain-specific differences in representation among M. smithii strains.

Alternatively, the nucleic acid or nucleic acids derived from a M. smithii genome may be selected from the group of nucleic acids comprising nucleic acid sequences that are expressed in vivo by M. smithii while residing in the gastrointestinal tract of a subject. In another embodiment, the nucleic acid or nucleic acids may be selected from the group of nucleic acids comprising nucleic acid sequences that are expressed by M. smithii while residing in the gastrointestinal tract of a subject, and whose expression levels are not affected by the presence of actively fermenting bacteria. In another embodiment, the nucleic acid or nucleic acids may be selected from the group of nucleic acids comprising nucleic acid sequences that are expressed by M. smithii while residing in the gastrointestinal tract of a subject, and whose expression levels are affected by the presence of actively fermenting bacteria. The in vivo expression levels of a nucleic acid may be determined by methods known in the art, including RT-PCR. In yet another embodiment, the nucleic acid or nucleic acids may be selected from the group of nucleic acids that encode the M. smithii transcriptome or metabolome. In yet another embodiment, the nucleic acid or nucleic acids may be selected from the group of nucleic acids whose expression level differ between strains of M. smithii when the bacteria are grown in vitro or in vivo under similar conditions.

The biomolecule may also be a polypeptide derived from a M. smithii proteome. A polypeptide derived from the M. smithii proteome is a polypeptide that is encoded by at least a portion of a nucleic acid sequence selected from the nucleic acid sequences listed in Table A, Table B or Table D. The polypeptide may comprise fewer than 10, at least 10, at least 20, at least 30, at least 40, at least 50, at least 60, at least 70, at least 80, at least 90, at least 100, at least 150, at least 200, or more than 200 amino acids encoded by a nucleic acid sequence selected from the nucleic acid sequences listed in Table A, Table B or Table D. One embodiment of the invention is an array comprising a substrate, the substrate having disposed thereon at least one polypeptide, wherein the polypeptide is encoded by a nucleic acid sequence selected from the nucleic acid sequences listed in Table A. Another embodiment of the invention is an array comprising a substrate, the substrate having disposed thereon at least one polypeptide, wherein the polypeptide is encoded by a nucleic acid sequence selected from the nucleic acid sequences listed in Table B. Still another embodiment of the invention is an array comprising a substrate, the substrate having disposed thereon at least one polypeptide, wherein the polypeptide comprises an amino acid sequence selected listed in Table C. A different embodiment of the invention is an array comprising a substrate, the substrate having disposed thereon at least one polypeptide, wherein the polypeptide is encoded by a nucleic acid sequence selected from the nucleic acid sequences listed in Table D.

In one embodiment, the polypeptide or polypeptides may be selected from the group of polypeptides comprising polypeptide sequences that are conserved amoung M. smithii strains, but divergent from a corresponding polypeptide of the subject. The terms conserved and divergent are used as defined above. In certain embodiments, the polypeptide or polypeptides are selected from the group comprising polypeptides absent from the subject gut microbiome or genome. In another embodiment, the polypeptide or polypeptides may be selected from the group of polypeptides comprising polypeptide sequences with greater than about 75% but less than about 99% identity to a correlating polypeptide from the subject gut microbiome or genome. In yet another embodiment, the polypeptide or polypeptides may be selected from the group of polypeptides comprising polypeptide sequence with greater than about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, or 98% identity to a correlating polypeptide from the subject gut microbiome or genome. In one embodiment, for instance, the polypeptide may be encoded by a nucleic acid designated absent or divergent in Table 2. Percent identity may be determined as discussed below.

Alternatively, the polypeptide or polypeptides derived from a M. smithii proteome may be encoded by a nucleic acid selected from the group of nucleic acids comprising nucleic acid sequences that are expressed in vivo by M. smithii while residing in the gastrointestinal tract of a subject. In another embodiment, the polypeptide or polypeptides may be encoded by a nucleic acid selected from the group of nucleic acids comprising nucleic acid sequences that are expressed by M. smithii while residing in the gastrointestinal tract of a subject, and whose expression levels are not affected by the presence of actively fermenting bacteria. In still another embodiment, the polypeptide or polypeptides may be encoded by a nucleic acid selected from the group of nucleic acids comprising nucleic acid sequences that are expressed by M. smithii while residing in the gastrointestinal tract of a subject, and whose expression levels are affected by the presence of actively fermenting bacteria. In yet another embodiment, the polypeptide or polypeptides may be encoded by a nucleic acid selected from the group of nucleic acids that encode the M. smithii transcriptome or metabolome.

The array may alternatively be comprised of biomolecules from the genome or proteome of M. smithii that are indicative of an obese subject microbiome. Alternatively, the array may be comprised of biomolecules from the genome or proteome of M. smithii that are indicative of a lean subject microbiome. A biomolecule is “indicative” of an obese or lean microbiome if it tends to appear more often in one type of microbiome compared to the other. Such differences may be quantified using commonly known statistical measures, such as binomial tests. An “indicative” biomolecule may be referred to as a “biomarker.”

Additionally, the array may be comprised of biomolecules from the genome or proteome of M. smithii that are modulated in the obese subject microbiome compared to the lean subject microbiome. As used herein, “modulated” may refer to a biomolecule whose representation or activity is different in an obese subject microbiome compared to a lean subject microbiome. For instance, modulated may refer to a biomolecule that is enriched, depleted, up-regulated, down-regulated, degraded, or stabilized in the obese subject microbiome compared to a lean subject microbiome. In one embodiment, the array may be comprised of a biomolecule enriched in the obese subject microbiome compared to the lean subject microbiome. In another embodiment, the array may be comprised of a biomolecule depleted in the obese subject microbiome compared to the lean subject microbiome. In yet another embodiment, the array may be comprised of a biomolecule up-regulated in the obese subject microbiome compared to the lean subject microbiome. In still another embodiment, the array may be comprised of a biomolecule down-regulated in the obese subject microbiome compared to the lean subject microbiome. In still yet another embodiment, the array may be comprised of a biomolecule degraded in the obese subject microbiome compared to the lean subject microbiome. In an alternative embodiment, the array may be comprised of a biomolecule stabilized in the obese subject microbiome compared to the lean subject microbiome.

Additionally, the biomolecule may be at least 80, 85, 90, or 95% homologous to a biomolecule derived from Tables A-D. In one embodiment, the biomolecule may be at least 80, 81, 82, 83, 84, 85, 86, 87, 88, or 89% homologous to a biomolecule derived from Table A. In another embodiment, the biomolecule may be at least 90, 91, 92, 93, 94, 95, 96, 97, 98, 99, or 100% homologous to a biomolecule derived from Table A. In another embodiment, the biomolecule may be at least 80, 81, 82, 83, 84, 85, 86, 87, 88, or 89% homologous to a biomolecule derived from Table B. In another embodiment, the biomolecule may be at least 90, 91, 92, 93, 94, 95, 96, 97, 98, 99, or 100% homologous to a biomolecule derived from Table B. In another embodiment, the biomolecule may be at least 80, 81, 82, 83, 84, 85, 86, 87, 88, or 89% homologous to a biomolecule derived from Table C. In another embodiment, the biomolecule may be at least 90, 91, 92, 93, 94, 95, 96, 97, 98, 99, or 100% homologous to a biomolecule derived from Table C. In another embodiment, the biomolecule may be at least 80, 81, 82, 83, 84, 85, 86, 87, 88, or 89% homologous to a biomolecule derived from Table D. In another embodiment, the biomolecule may be at least 90, 91, 92, 93, 94, 95, 96, 97, 98, 99, or 100% homologous to a biomolecule derived from Table D.

In determining whether a biomolecule is substantially homologous or shares a certain percentage of sequence identity with a sequence of the invention, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See http://www.ncbi.nlm.nih.gov for more details.

Furthermore, the biomolecules used for the array may be labeled. One skilled in the art understands that the type of label selected depends in part on how the array is being used. Suitable labels may include fluorescent labels, chromagraphic labels, chemi-luminescent labels, FRET labels, etc. Such labels are well known in the art.

### II. Use of the Arrays

The arrays may be utilized in several suitable applications. For example, the arrays may be used in methods for detecting association between a biomolecule of the array and a compound in a sample. In this context, compound refers to a nucleic acid, a protein, a lipid, or chemical compound. This method typically comprises incubating a sample with the array under conditions such that the compounds comprising the sample may associate with the biomolecules attached to the array. The association is then detected, using means commonly known in the art, such as fluorescence. “Association,” as used in this context, may refer to hybridization, covalent binding, ionic binding, hydrogen binding, van der Waals binding, and dated binding. A skilled artisan will appreciate that conditions under which association may occur will vary depending on the biomolecules, the compounds, the substrate, and the detection method utilized. As such, suitable conditions may have to be optimized for each individual array created.

In one embodiment, the array may be used as a tool in methods to determine whether a compound has efficacy for modulating a gene product of M. smithii. In certain embodiments, the array may be used as a tool in methods to determine whether a compound has efficacy for modulating a gene product of M. smithii while M. smithii is residing in the gastrointestinal tract of a subject. Typically, such a method comprises comparing a plurality of biomolecules from either the M. smithii genome or proteome before and after administration of a compound for modulating a gene product of M. smithii, such that if the abundance of a biomolecule that correlates with the gene product is modulated, the compound is efficacious in modulating a gene product of M. smithii. The array may also be used to quantitate the plurality of biomolecule's of M. smithii's genome or proteome before and after administration of a compound. The abundance of each biomolecule in the plurality may then be compared to determine if there is a decrease in the abundance of biomolecules associated with the compound. In other embodiments, the array may be used to quantify the levels of M. smithii in an obese subject prior to, during, or after treatment for obesity. Alternatively, the array may be used to quantify the levels of M. smithii in an underfed individual prior to, during, or after implementation of dietary recommendations designed to increase nutrient and energy harvest.

In a further embodiment, the array may be used as a tool in methods to determine the identity of an M. smithii strain present in a subject's microbiome. Typically, such a method comprises collecting a sample from a subject and using an array of the invention to determine the presence, absence or abundance of an ALP gene product in the sample, and determining whether a particular strain is present in the sample based on the presence, absence or abundance of an ALP gene product.

In still a further embodiment, the array may be used as a tool in methods to determine whether a compound has efficacy for treatment of weight gain or a weight gain related disorder in a subject. Typically, such a method comprises comparing a plurality of biomolecules of M. smithii's genome or proteome before and after administration of a compound for the treatment of weight gain or a weight gain related disorder, such that if the abundance of biomolecules associated with weight gain decreased after treatment, the compound is efficacious in treating weight gain in a subject.

In still a further embodiment, the array may be used as a tool in methods to determine whether a compound has efficacy for treatment of weight loss or a weight loss related disorder in a subject. Typically, such a method comprises comparing a plurality of biomolecules of M. smithii's genome or proteome before and after administration of a compound for the treatment of weight loss or a weight loss related disorder, such that if the abundance of biomolecules associated with weight loss decreased after treatment, the compound is efficacious in treating weight loss in a subject.

The present invention also encompasses M. smithii gene profiles. Generally speaking, a gene profile is comprised of a plurality of values with each value representing the abundance of a biomolecule derived from either the M. smithii genome or proteome. The abundance of a biomolecule may be determined, for instance, by sequencing the nucleic acids of the M. smithii genome as detailed in the examples. This sequencing data may then be analyzed by known software to determine the abundance of a biomolecule in the analyzed sample. An M. smithii gene profile may comprise biomolecules from more than one M. smithii strain. The abundance of a biomolecule may also be determined using an array described above. For instance, by detecting the association between compounds comprising an M. smithii derived sample and the biomolecules comprising the array, the abundance of M. smithii biomolecules in the sample may be determined.

A profile may be digitally-encoded on a computer-readable medium. The term “computer-readable medium” as used herein refers to any medium that participates in providing instructions to a processor for execution. Such a medium may take many forms, including but not limited to non-volatile media, volatile media, and transmission media. Non-volatile media may include, for example, optical or magnetic disks. Volatile media may include dynamic memory. Transmission media may include coaxial cables, copper wire and fiber optics. Transmission media may also take the form of acoustic, optical, or electromagnetic waves, such as those generated during radio frequency (RF) and infrared (IR) data communications. Common forms of computer-readable media include, for example, a floppy disk, a flexible disk, hard disk, magnetic tape, or other magnetic medium, a CD-ROM, CDRW, DVD, or other optical medium, punch cards, paper tape, optical mark sheets, or other physical medium with patterns of holes or other optically recognizable indicia, a RAM, a PROM, and EPROM, a FLASH-EPROM, or other memory chip or cartridge, a carrier wave, or other medium from which a computer can read.

A particular profile may be coupled with additional data about that profile on a computer readable medium. For instance, a profile may be coupled with data about what therapeutics, compounds, or drugs may be efficacious for that profile. Conversely, a profile may be coupled with data about what therapeutics, compounds, or drugs may not be efficacious for that profile. Alternatively, a profile may be coupled with known risks associated with that profile. Non-limiting examples of the type of risks that might be coupled with a profile include disease or disorder risks associated with a profile. The computer readable medium may also comprise a database of at least two distinct profiles.

Profiles may be stored on a computer-readable medium such that software known in the art and detailed in the examples may be used to compare more than one profile.

Another aspect of the invention is a method for selecting a compound that has efficacy for modulating a gene product of M. smithii present in the gastrointestinal tract of a subject. The method generally comprises comparing an M. smithii gene profile to a gene profile of the subject and identifying a gene product of the M. smithii gene profile that is divergent from a corresponding gene product of the subject gene profile, or absent in the gene profile of the subject. Next the method comprises selecting a compound that modulates the M. smithii gene product, but does not substantially modulate the corresponding gene product of the subject. In a further embodiment, the compound also does not substantially modulate the corresponding gene product of an archaeon other than M. smithii, or a non-archaeal microbe, in the gastrointestinal tract of the subject. The compound may for instance, inhibit or promote the growth of M. smithii. The compound may also decrease or increase the efficiency of carbohydrate metabolism in the subject. Accordingly, the compound may also promote weight loss or weight gain in the subject.

Another further aspect of the invention is a method for selecting a compound that has efficacy for modulating a gene product of M. smithii present in the gastrointestinal tract of a subject. The method comprises comparing an M. smithii gene profile to a gene profile of the subject and identifying a gene product of the M. smithii gene profile that is divergent from a corresponding gene product of the subject gene profile, or absent in the gene profile of the subject. Next the method comprises selecting a compound that can be administered so as to modulate the M. smithii gene product, but not substantially modulate the corresponding gene product of the subject. In a further embodiment, the administered compound also does not substantially modulate the corresponding gene product of an archaeon other than M. smithii, or a non-archaeal microbe, in the gastrointestinal tract of the subject. The compound may be administered, for instance, so as to inhibit or promote the growth of M. smithii. The compound may also be administered so as to decrease or increase the efficiency of carbohydrate metabolism in the subject. Accordingly, the compound may also be administered so as to promote weight loss or weight gain in the subject.

The present invention also encompasses a kit for evaluating a compound, therapeutic, or drug. Typically, the kit comprises an array and a computer-readable medium. The array may comprise a substrate having disposed thereon at least one biomolecule that is derived from the M. smithii genome or proteome. In some embodiments, the array may comprise at least one biomolecule that is derived from the M. smithii metabolome or transcriptome. The computer-readable medium may have a plurality of digitally-encoded profiles wherein each profile of the plurality has a plurality of values, each value representing the abundance of a biomolecule derived from M. smithii detected by the array. The array may be used to determine a profile for a particular subject under particular conditions, and then the computer-readable medium may be used to determine if the profile is similar to known profile stored on the computer-readable medium. Non-limiting examples of possible known profiles include obese and lean profiles for several different subjects.

### III. Method of Promoting Weight Loss or Gain

A further aspect of the invention encompasses a method of promoting weight loss or gain. The method incorporates the discovery that modulating the Archaeon population of the gastrointestinal tract of a subject, of which M. smithii is a major component, modulates the efficiency and selectivity of carbohydrate metabolism. Furthermore, the method relies on applicants' discovery that certain M. smithii gene products are conserved amoung M. smithii strains, yet divergent (or absent) from the correlating gene products expressed by the subject's microbiome or genome. This divergence allows the selection of compounds to specifically modulate the M. smithii gene product, while substantially not modulating the subject's gene product, as described above.

By way of non-limiting example, weight loss may be promoted by administering an HMG-CoA reductase inhibitor to a subject. In an exemplary embodiment, the inhibitor will selectively inhibit the HMG-CoA reductase expressed by M. smithii and not the HMG-CoA reductase expressed by the subject. In another embodiment, a second HMG CoA-reductase inhibitor may be administered that selectively inhibits the HMG CoA-reductase expressed by the subject in lieu of the HMG-CoA reductase expressed by M. smithii. In yet another embodiment, an HMG-CoA reductase inhibitor that selectively inhibits the HMG-CoA reductase expressed by the subject may be administered in combination with an HMG-CoA reductase inhibitor that selectively inhibits the HMG-CoA reducase expressed by M. smithii. One means that may be utilized to achieve such selectivity is via the use of time-release formulations as discussed below. Compounds that inhibit HMG-CoA reductase are well known in the art. For instance, non-limiting examples include atorvastatin, pravastatin, rosuvastatin, and other statins.

### (a) Pharmaceutical Compositions

These compounds, for example HMG-CoA reductase inhibitors, may be formulated into pharmaceutical compositions and administered to subjects to promote weight loss. According to the present invention, a pharmaceutical composition includes, but is not limited to, pharmaceutically acceptable salts, esters, salts of such esters, or any other adduct or derivative which upon administration to a subject in need is capable of providing, directly or indirectly, a composition as otherwise described herein, or a metabolite or residue thereof, e.g., a prodrug.

The pharmaceutical compositions maybe administered by several different means that will deliver a therapeutically effective dose. Such compositions can be administered orally, parenterally, by inhalation spray, rectally, intradermally, intracisternally, intraperitoneally, transdermally, bucally, as an oral or nasal spray, or topically (i.e. powders, ointments or drops) in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. In an exemplary embodiment, the pharmaceutical composition will be administered in an oral dosage form. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

The amount of an HMG-CoA reductase inhibitor that constitutes an “effective amount” can and will vary. The amount will depend upon a variety of factors, including whether the administration is in single or multiple doses, and individual subject parameters including age, physical condition, size, and weight. Those skilled in the art will appreciate that dosages may also be determined with guidance from Goodman & Goldman's The Pharmacological Basis of Therapeutics, Ninth Edition (1996), Appendix II, pp. 1707-1711 and from Goodman & Goldman's The Pharmacological Basis of Therapeutics, Tenth Edition (2001), Appendix II, pp. 475-493.

### (b) Controlled Release Formulations

As described above, an HMG-CoA reductase inhibitor may be specific for the M. smithii enzyme, or for the subject's enzyme, depending, in part, on the selectivity of the particular inhibitor and the area the inhibitor is targeted for release in the subject. For example, an inhibitor may be targeted for release in the upper portion of the gastrointestinal tract of a subject to substantially inhibit the subject's enzyme. In contrast, the inhibitor may be targeted for release in the lower portion of the gastrointestinal tract of a subject, i.e., where M. smithii resides, then the inhibitor may substantially inhibit M. smithii's enzyme.

In order to selectively control the release of an inhibitor to a particular region of the gastrointestinal tract for release, the pharmaceutical compositions of the invention may be manufactured into one or several dosage forms for the controlled, sustained or timed release of one or more of the ingredients. In this context, typically one or more of the ingredients forming the pharmaceutical composition is microencapsulated or dry coated prior to being formulated into one of the above forms. By varying the amount and type of coating and its thickness, the timing and location of release of a given ingredient or several ingredients (in either the same dosage form, such as a multi-layered capsule, or different dosage forms) may be varied.

The coating can and will vary depending upon a variety of factors, including, the particular ingredient, and the purpose to be achieved by its encapsulation (e.g., time release). The coating material may be a biopolymer, a semi-synthetic polymer, or a mixture thereof. The microcapsule may comprise one coating layer or many coating layers, of which the layers may be of the same material or different materials. In one embodiment, the coating material may comprise a polysaccharide or a mixture of saccharides and glycoproteins extracted from a plant, fungus, or microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, agar, alginates, chitosans, or gellan gum. In another embodiment, the coating material may comprise a protein. Suitable proteins include, but are not limited to, gelatin, casein, collagen, whey proteins, soy proteins, rice protein, and corn proteins. In an alternate embodiment, the coating material may comprise a fat or oil, and in particular, a high temperature melting fat or oil. The fat or oil may be hydrogenated or partially hydrogenated, and preferably is derived from a plant. The fat or oil may comprise glycerides, free fatty acids, fatty acid esters, or a mixture thereof. In still another embodiment, the coating material may comprise an edible wax. Edible waxes may be derived from animals, insects, or plants. Non-limiting examples include beeswax, lanolin, bayberry wax, carnauba wax, and rice bran wax. The coating material may also comprise a mixture of biopolymers. As an example, the coating material may comprise a mixture of a polysaccharide and a fat.

In an exemplary embodiment, the coating may be an enteric coating. The enteric coating generally will provide for controlled release of the ingredient, such that drug release can be accomplished at some generally predictable location in the lower intestinal tract below the point at which drug release would occur without the enteric coating. In certain embodiments, multiple enteric coatings may be utilized. Multiple enteric coatings, in certain embodiments, may be selected to release the ingredient or combination of ingredients at various regions in the lower gastrointestinal tract and at various times.

The enteric coating is typically, although not necessarily, a polymeric material that is pH sensitive. A variety of anionic polymers exhibiting a pH-dependent solubility profile may be suitably used as an enteric coating in the practice of the present invention to achieve delivery of the active to the lower gastrointestinal tract. Suitable enteric coating materials include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the trade name “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In one embodiment, the coating may comprise plant polysaccharides that can only be digested in the distal gut by the microbiota. For instance, a coating may comprise pectic galactans, polygalacturonates, arabinogalactans, arabinans, or rhamnogalacturonans. Combinations of different coating materials may also be used to coat a single capsule.

The thickness of a microcapsule coating may be an important factor in some instances. For example, the “coating weight,” or relative amount of coating material per dosage form, generally dictates the time interval between oral ingestion and drug release. As such, a coating utilized for time release of the ingredient or combination of ingredients into the gastrointestinal tract is typically applied to a sufficient thickness such that the entire coating does not dissolve in the gastrointestinal fluids at pH below about 5, but does dissolve at pH about 5 and above. The thickness of the coating is generally optimized to achieve release of the ingredient at approximately the desired time and location.

As will be appreciated by a skilled artisan, the encapsulation or coating method can and will vary depending upon the ingredients used to form the pharmaceutical composition and coating, and the desired physical characteristics of the microcapsules themselves. Additionally, more than one encapsulation method may be employed so as to create a multi-layered microcapsule, or the same encapsulation method may be employed sequentially so as to create a multi-layered microcapsule. Suitable methods of microencapsulation may include spray drying, spinning disk encapsulation (also known as rotational suspension separation encapsulation), supercritical fluid encapsulation, air suspension microencapsulation, fluidized bed encapsulation, spray cooling/chilling (including matrix encapsulation), extrusion encapsulation, centrifugal extrusion, coacervation, alginate beads, liposome encapsulation, inclusion encapsulation, colloidosome encapsulation, sol-gel microencapsulation, and other methods of microencapsulation known in the art. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6^{th} Ed. (Media, Pa.: Williams & Wilkins, 1995).

### DEFINITIONS

The term “activity of the microbiota population” refers to the microbiome's ability to harvest energy.

An “effective amount” is a therapeutically-effective amount that is intended to qualify the amount of agent that will achieve the goal of modulating an M. smithii gene product, promoting weight loss, or promoting weight gain.

As used herein, “gene product” refers to a nucleic acid derived from a particular gene, or a polypeptide derived from a particular gene. For instance, a gene product may be a mRNA, tRNA, rRNA, cDNA, peptide, polypeptide, protein, or metabolite.

“Metabolome” as used herein is defined as the network of enzymes and their substrates and biochemical products, which operate within subject or microbial cells under various physiological conditions.

As used herein, the term “pharmaceutically acceptable salt” refers to those salts which are, within the scope of sound medical judgment, suitable for use in contact with the tissues of humans and other subjects without undue toxicity, irritation, allergic response and the like, and are commensurate with a reasonable benefit/risk ratio. Pharmaceutically acceptable salts are well known in the art. For example, S. M. Berge, et al. describe pharmaceutically acceptable salts in detail in J. Pharmaceutical Sciences, 66: 1 19 (1977), incorporated herein by reference. The salts can be prepared in situ during the final isolation and purification of the composition of the invention, or separately by reacting the free base function with a suitable organic acid. Non-limiting examples of pharmaceutically acceptable, nontoxic acid addition salts are salts of an amino group formed with inorganic acids such as hydrochloric acid, hydrobromic acid, hydroionic acid, nitric acid, carbonic acid, phosphoric acid, sulfuric acid and perchloric acid.

As used herein, the “subject” may be, generally speaking, an organism capable of supporting M. smithii in its gastrointestinal tract. For instance, the subject may be a rodent or a human. In one embodiment, the subject may be a rodent, i.e. a mouse, a rat, a guinea pig, etc. In an exemplary embodiment, the subject is human.

“Transcriptome” as used herein is defined as the network of genes that are being actively transcribed into mRNA in subject or microbial cells under various physiological conditions.

The phrase “weight gain related disorder” includes disorders resulting from, at least in part, obesity. Representative disorders include metabolic syndrome, type II diabetes, hypertension, cardiovascular disease, and nonalcoholic fatty liver disease. The phrase “weight loss related disorder” includes disorders resulting from, at least in part, weight loss. Representative disorders include malnutrition and cachexia.

As various changes could be made in the above compounds, products and methods without departing from the scope of the invention, it is intended that all matter contained in the above description and in the examples given below, shall be interpreted as illustrative and not in a limiting sense.

### EXAMPLES

The following examples illustrate various iterations of the invention.

### Materials and Methods for Examples 1-5

### Genome Sequencing and Annotation

Methanobrevibacter smithii strain PS (ATCC 35061) was grown as described below for 6d at 37° C. DNA was recovered from harvested cell pellets using the QIAGEN Genomic DNA Isolation kit with mutanolysin (1 unit/mg wet weight cell pellet; Sigma) added to facilitate lysis of the microbe. An ABI 3730xl instrument was used for paired end-sequencing of inserts in a plasmid library (average insert size 5 Kb; 42,823 reads; 11.6×-fold coverage), and a fosmid library (average insert size of 40 Kb; 7,913 reads; 0.6×-fold coverage). Phrap and PCAP (Huang et al. (2003) Genome Res 13:2164-70) were used to assemble the reads. A primer-walking approach was used to fill-in sequence gaps. Physical gaps and regions of poor quality (as defined by Consed; Gordon et al., (1998) Genome Res. 8, 195-202) were resolved by PCR-based re-sequencing. The assembly's integrity and accuracy was verified by clone constraints. Regions containing insufficient coverage or ambiguous assemblies were resolved by sequencing spanning fosmids. Sequence inversions were identified based on inconsistency of constraints for a fraction of read pairs in those regions. The final assembly consisted of 12.6× sequence coverage with a Phred base quality value 40. Open-reading frames (ORFs) were identified and annotated as described below.

### Quantitative RT-PCR Analyses

All experiments using mice were performed using protocols approved by the animal studies committee of Washington University. Gnotobiotic male mice belonging to the NMRI inbred strain (n=5-6/group/experiment) were colonized with either M. smithii (14d) or B. thetaiotaomicron (28d) alone, or first with B. thetaiotaomicron for 14d followed by co-colonization with M. smithii. All mice were sacrificed at 12 weeks of age. Cecal contents from each mouse were flash frozen, and stored at −80° C. RNA was extracted from an aliquot of the harvested cecal contents (100-300 mg) and used to generate cDNA for qRT-PCR assays. qRT-PCR data were normalized to 16S rRNA (ΔΔC_{T} method) prior to comparing treatment groups. PCR primers are listed in Table 14. All amplicons were 100-150 bp.

### Biochemical Assays

Perchloric acid-, hydrochloric acid-, and alkali extracts of freeze dried cecal contents were prepared, and established pyridine nucleotide-linked microanalytic assays (Passonneau et al., (1993) Enzymatic Analysis: A practical guide) used to measure metabolites.

### Microbes and Culturing

All M. smithii strains [PS (ATCC 35061), ALI (DSMZ 2375), B181 (DSMZ 11975), and F1 (DSMZ 2374)] were cultivated in 125 ml serum bottles containing 15 ml MBC medium supplemented with 3 g/L formate, 3 g/L acetate, and 0.3 mL of a freshly prepared anaerobic solution of filter-sterilized 2.5% Na₂S (Samuel et al., (2006) PNAS 103:10011-6). The remaining volume in the bottle (headspace) contained a 4:1 mixture of H₂ and CO₂: the headspace was replenished every 1-2d for a 6d growth at 37° C.

M. smithii PS was also cultured in a BioFlor-110 batch fermentor with dual 1.5 L fermentation vessels (New Brunswick Scientific). Each vessel contained 750 ml of supplemented MBC medium. One hour prior to inoculation, 7.5 ml of sterile 2.5% Na₂S solution was added to the vessel, followed by one half of the contents of a serum bottle culture that had been harvested on day 5 of growth. Microbes were then incubated at 37° C. under a constant flow of H₂/CO₂ (4:1) (agitation setting, 250 rpm). One milliliter of a sterile solution of 2.5% Na₂S was added daily.

Colonization of Germ-Free Mice with M. smithii PS with and without B. thetaiotaomicron VPI-5482

Mice belonging to the NMRI/KI inbred strain (Bry et al., (1996) Science 273:1380-3) were housed in gnotobiotic isolators (Hooper et al., (2002) Mol Cell Micro 31:559-589) where they were maintained under a strict 12 h light cycle (lights on at 0600 h) and fed a standard, autoclaved, polysaccharide-rich chow diet (B&K Universal, East Yorkshire, UK) ad libitum. Each mouse was inoculated at age 8 weeks with a single gavage of 10⁸ microbes/strain [B. thetaiotaomicron was harvested from an overnight culture in TYG medium (Sonnenburg et al., Science 307:1955-9); M. smithii from serum bottles containing MBC medium after a 5d incubation at 37° C. (Samuel et al., (2006) PNAS 103:10011-6)]. For a given experiment, the same preparation of cultured microbes was used for mono-association (single species added) and co-colonization (both species added).

Immediately after animals were sacrificed, cecal contents were recovered for preparation of DNA, RNA and biochemical studies (n=5 mice/treatment group/experiment; n=3 independent experiments). Colonization density was assessed using a qPCR-based assay employing species-specific primers, as described in Samuel et al., (2006) PNAS 103:10011-6.

### Genome Annotation

M. smithii genes were identified by comparing outputs from GLIMMER v.3.01 (Delcher et al., (1999) Nucleic Acids Res 27:4636-41), CRITICA v.1.05b (Badger et al., (1999) Mol Biol Evol 16:512-24), and GeneMarkS v.2.1 (Besemer et al. (2001) Nucleic Acids Res 29:2607-18). WUBLAST (http://blast.wustl.edu/) was then used to identify all ORFs with significant hits to the NR database (as of Dec. 1, 2006). ORFs containing <30 codons and without significant homology (e-value threshold of 10⁻⁵) to other proteins, were eliminated. rRNA and tRNA genes were identified using BLASTN and tRNA-Scan (Lowe et al., (1997) Nucleic cids Res 25:955-64). Annotation of the predicted proteome of M. smithii was completed by using BLAST homology searches against public databases, and domain analysis with Pfam (http://pfam.janelia.org/) and InterProScan [release 12.1; (Apweiler et al., Nucleic Acids Res 29:37-40)]. Functional classifications were made based on GO terms assigned by InterProScan and homology searches against COGs (Tatusov et al., (2001) Nucleic Acids Res 29:22-8), followed by manual curation. Metabolic pathways were constructed based on KEGG (Kanehisa et al., (2004) Nucleic Acids Res 32:D277-80) and MetaCyc [(Caspi et al., (2006) Nucleic Acids Res 34:D511-6); http://metacyc.org/)]. Glycosyltransferases (GT) were categorized according to CAZy [http://www.cazy.org; (Coutinho et al., (1999) Recent Advances in Carbohydrate Bioengineering p. 3-12)]. Putative prophage genes were identified using two independent approaches: (i) BLASTN of predicted M. smithii ORFs against a database of all known phage sequences (http://phage.sdsu.edu/phage); and (ii) Hidden Markov Model (HMM)-based analysis using Phage_Finder (Fouts (2006) Nucleic Acids Res 34:5839-51).

### Comparative Genomic Analyses

GO term assignments—The number of genes in each archaeal genome that were assigned to each GO term, or to its parents in the GO hierarchy [version available on Jun. 6, 2006; (Ashburner et al., (2000) Nat Genet. 25:25-9)] were totaled. All terms assigned to at least five genes in a given genome were then subjected to statistical tests for overrepresentation, and all terms with a total of five genes across all tested genomes for under-representation, using a binomial comparison reference set (see Table 6). Genes that could not be assigned to a GO category were excluded from the reference sets. A false discovery rate of <0.05 was set for each comparison (Benjamini et al., (1995) J of the Royal Statistical Society B 57:289-300). All tests were implemented using the Math::CDF Perl module (E. Callahan, Environmental Statistics, Fountain City, Wis.; available at http://www.cpan.org/), and scripts written in Perl.

Percent identity comparisons—The M. smithii PS genome sequence was compared to the M. stadtmanae genome (Fricke et al., (2006) J Bacteriol 188:642-58) and a 78 Mb metagenomic dataset of the human fecal microbiome (Gill et al., (2006) Science 312:1355-9) using NUCmer (part of MUMmer v.3.19 package; (Kurtz et al., Genome Biol 5:R12), and a percent identity plot was generated using Mummerplot.

Genomic synteny—Comparisons of synteny between M. smithii and M. stadtmanae were completed using the Artemis Comparison Tool (Carver et al., (2005) Bioinformatics 21:3422-3) set to tBLASTX and the most stringent confidence level.

M. smithii interaction network analyses—All M. smithii COGs were submitted to the STRING database (http://string.embl.de/; (von Mering et al., (2003) Nucleic Acids Res 31:258-61) to create predicted interaction networks (0.95 confidence interval). The program Medusa (Hooper et al., (2005) Bioinformatics 21:4432-3) was then used to organize the networks and color the nodes based on their conservation in M. smithii's proteome (mutual best BLASTP hits with e-values<10⁻²⁰ to the other Methanobacteriales genomes).

Clustering of adhesin-like proteins—M. smithii and M. stadtmanae ALPs were first aligned using CLUSTALW (v.1.83; (Chenna et al., (2003) Nucleic Acids Res 31:3497-500)). To retain the highest level of discrimination between the proteins, the alignment was subsequently converted into a nucleotide alignment using PAL2NAL (Suyama et al., (2006) Nucleic Acids Res 34:W609-12). The resulting alignment was used to create a maximum likelihood tree with RA×ML [Randomized accelerated maximum likelihood for high performance computing [RA×ML-VI-HPC, v2.2.1; (Stamatakis (2006) Bioinformatics 22:2688-90)] first using the GTR+CAT approximation method for rapid generation of tree topology, followed by the GTR+gamma evolutionary model for determination of likelihood values. ModelTest (v3.7; http://darwin.uvigo.es/software/modeltest.html) also identified GTR+gamma as the most appropriate evolutionary model for the dataset. Bootstrap values were determined from 100 neighbor-joining trees in Paup (v. 4.0b10, http://paup.csit.fsu.edu/). Tree visualization was completed with TreeView (Page (1996) Comput Appl Biosci 12:357-8).

Functional Genomic Analysis of M. smithii Gene Expression in Gnotobiotic Mice

RNA isolation—100-300 mg aliquots of frozen cecal contents from each gnotobiotic mouse was added to 2 ml tubes containing 250 μl of 212-300 μm-diameter acid-washed glass beads (Sigma), 500 μl of buffer A (200 mM NaCl, 20 mM EDTA), 210 μl of 20% SDS, and 500 μl of a mixture of phenol:chloroform:isoamyl alcohol (125:24:1; pH 4.5; Ambion). Samples were lysed using a bead beater (BioSpec; ‘high’ setting for 5 min at room temperature) and cellular debris was pelleted by centrifugation (10,000×g at 4° C. for 3 min). The extraction was repeated by adding another 500 μL of phenol:chloroform:isoamyl alcohol to the aqueous supernatant. RNA was precipitated from the pooled aqueous phases, resuspended in 100 μl nuclease-free water (Ambion), 350 μl Buffer RLT (QIAGEN) was added, and RNA further purified using the RNeasy mini kit (QIAGEN).

Analysis of the Sialic Acid Production by M. smithii

Reverse-phase HPLC analysis of cellular extracts—M. smithii was cultured in MBC medium, in a batch fermenter, to stationary phase (6d incubation). Cells were collected by centrifugation, washed three times in PBS, snap frozen in liquid nitrogen, and stored at −80° C. Sialic acid content was assayed using established protocols (Manzi et al., (1995) Current Protocols in Molecular Biology)). Briefly, sialic acids were liberated by homogenization of the cell pellet (˜30-50 mg wet weight) in 0.5 ml of 2M acetic acid with subsequent incubation of the homogenate for 3 h at 80° C. Samples were filtered through Microcon 10 filters (Millipore) and the filtrate, containing free sialic acid, was dried (speed-vacuum). The released sialic acid was derivatized with DMB (1,2-diamino-4,5-methylene-dioxybenzene) to yield a fluorescent adduct, which was analyzed by C18 reverse phase high-pressure liquid chromatography (RP-HPLC; Dionex DX-600 workstation). Sialic acid was quantified by comparison to known amounts of derivatized standards [N-acetylneuraminic acid (Neu5Ac) and Nglycolylneuraminic acid (Neu5Gc)], and blanks (buffer alone).

Histochemical studies—M. smithii strains PS and F1 were grown in MBC as above. Bacteroides thetaiotaomicron VPI-5482, and Bifidobacterium longum NCC2705 were grown under anaerobic conditions in TYG medium to stationary phase and used as negative controls. Escherichia coli strain K92 (ATCC 35860), which is known to produce sialic acid (Egan et al., (1977) Biochemistry 16:3687-92), was incubated in 1419 medium (ATCC) to stationary phase and used as a positive control. All strains were fixed in 1.5 ml conical plastic tubes in either 4% paraformaldehyde or 100% ethanol for at least 8 h at 4° C. Samples were then washed with PBS and stored at −20° C. in 50% ethanol, 20 mM Tris and 0.1% IGEPAL CA-630 (Sigma; prepared in deionized water) until assayed. Samples were diluted in deionized water, placed on coated glass slides (Cel-Line/Erie Scientific Co.), air-dried, dehydrated in graded ethanols (50%, 80%, 100%), treated with blocking buffer (0.3% Triton X-100, 1% BSA in PBS; 30 min at room temperature), and then incubated with 10 μg/ml fluorescein-labeled Sambucus nigra lectin (SNA; Vector Laboratories; specificity, Neu5Acα-2,6Gal/GalNAc epitopes) for 1 h at room temperature. Slides were subsequently washed with PBS, stained with 4′,6-diamidino-2-phenylindole (DAPI, 2 μg/ml; 5 min at room temperature), washed with de-ionized water, and mounted in PBS/glycerol. Slides were visualized with an Olympus BX41 microscope and photographed using a Q Imaging QICAM camera and OpenLab software (Improvision, Inc., v.3.1.5).

Transmission Electron Microscopy (TEM) of M. smithii.

Cells were harvested at day 6 of growth in the batch fermentor, and cellular morphology was defined by TEM using methods identical to those described previously for B. thetaiotaomicron (Sonnenburg et al., (2005) Science 307:1955-9). TEM studies of M. smithii present in the ceca of gnotobiotic mice that had been colonized for 14d with the archaeon were conducted using the same protocol.

Microanalytic Biochemical Analyses of Cecal Samples Recovered from Gnotobiotic Mice

Extraction of metabolites from cecal contents—For measurement of ammonia and urea levels, perchloric acid extracts were prepared from 2 mg of freeze-dried cecal contents. [Contents were collected with a 10 μl inoculation loop, quick frozen in liquid nitrogen, and lyophilized at −35° C.] The lyophilized sample was homogenized in 0.2 ml of 0.3M perchloric acid at 1° C.

For the remaining metabolites, alkali and acid extracts were prepared from 4 mg of dried cecal samples that were homogenized in 0.4 ml 0.2M NaOH at 1° C. For the alkali extract, an 80 μl aliquot was removed, heated for 20 min at 80° C. and then neutralized with 80 μl of 0.25M HCl and 100 mM Tris base. For the acid extract, a 60 μl aliquot was removed and added to 20 μl 0.7M HCl, heated for 20 min at 80° C., and then neutralized with 40 μl 100 mM Tris base. Protein content was determined in the alkali extracts using the Bradford method (Bio Rad).

Metabolite assays—The sample concentrations for ammonium and urea were high enough so that direct fluorometric measurements could be used for detection. However, to measure the low sample concentrations for asparagine, glutamate, glutamine, α-ketoglutarate and ethanol, protocols were adapted from previously established pyridine nucleotide-linked assays, an “oil well” technique, and enzymatic cycling amplification (Passonneau et al., (1993) Enzymatic Analysis: A Practical Guide). All chemicals and enzymes were from Sigma unless otherwise noted.

Ammonium and Urea: For measurement of ammonium, a 20 μl aliquot of a perchloric acid extract of a given sample of cecal contents was added to 1 ml of a solution containing 50 mM imidazole HCl (pH 7.0), 0.2 mM α-ketoglutarate, 0.5 mM EDTA, 0.02% BSA, 10 μM NADH, and 10 μg/ml beef liver glutamate dehydrogenase (in glycerol; specific activity, 40 units/mg protein). Following a 40 min incubation at 24° C., fluorescence was measured using a Ratio-3 system filter fluorometer (Farrand Optical Components and Instruments, Valhalla, N.Y.; excitation at 360 nm; emission at 460 nm). Sample blanks were run that lacked added glutamate dehydrogenase. Ammonium acetate standards were carried throughout all steps.

To measure urea concentrations, 2 μl of a 50 mg/ml solution of Jack bean urease (50 units/mg) was added to the same sample used to determine ammonium levels. Following a 40 min incubation at 24° C., urea levels were defined based on a further reduction in fluorescence. Control sample blanks lacked added urease. Reference urea standards were carried throughout all steps.

Asparagine: A 0.5 μl aliquot of the alkali extract of a given sample of cecal contents was added to 0.5 μl of a solution containing 50 mM Trizma HCl (pH 8.7), 0.04% BSA, and 4 μg/ml E. coli asparaginase (160 units/mg protein). Sample blanks lacked added asparaginase. After a 30 min incubation at 24° C., 2 μl of a solution containing 50 mM Trizma HCl (pH 8.1), 10 μM α-ketoglutarate, 10 μM NADH, 4 mM freshly prepared ascorbic acid, 10 μg/ml of pig heart glutamic-oxalacetic transaminase (220 units/mg protein), plus 5 μg/ml beef heart malic dehydrogenase (2800 units/mg protein) was added, and the resulting mixture was incubated for 30 min at 24° C. One microliter of 0.25M HCl was then introduced. After a 10 min incubation at 24° C., a 2 μl aliquot of the reaction mixture was transferred to 0.1 ml of NAD cycling reagent for 20,000 cycles of amplification and the amplified product measured according to methods described by Passonneau and Lowry ((1993) Enzymatic Analysis: A Practical Guide). Sample blanks lacked added asparaginase. Reference asparagine standards were carried throughout all steps.

Glutamate and Glutamine: A 0.1 μl aliquot from an acid extract of a given sample of cecal contents was added to 0.1 μl of reagent containing 100 mM Na acetate (pH 4.9), 20 mM HCl, 0.4 mM EDTA and 50 μg/ml E. coli glutaminase (780 units/mg protein). Another 0.1 μl aliquot of the cecal contents was added to the same reagent in a parallel reaction that lacked added glutaminase (to measure glutamate alone). Following a 60 min incubation at 24° C., 2 μl of a solution containing 50 mM Tris acetate (pH 8.5), 0.1 mM NAD+, 0.1 mM ADP and 50 μg/ml beef liver glutamate dehydrogenase (120 units/mg protein; Roche) was added to both reaction mixtures, which were subsequently incubated for 30 min at 24° C. The reactions were terminated by addition of 1 μl of 0.2M NaOH and then heated for 20 min at 80° C. A 2 μl aliquot was subsequently transferred to 0.1 ml NAD cycling reagent and subjected to 20,000 cycles of amplification. Reference glutamine and glutamate standards were carried throughout all steps.

α-Ketoglutarate—A 0.5 μl aliquot from an given alkali extract was added to 0.5 μl of reagent containing 100 mM imidazole acetate (pH 6.5), 0.04% BSA, 50 mM ammonium acetate, 0.2 mM ADP, 4 mM ascorbic acid (freshly prepared), 40 μM NADH and 20 μg/ml beef liver glutamate dehydrogenase (120 units/mg protein; Roche). Following a 30 min incubation at 24° C., the reaction was terminated by adding 0.5 μl of 0.2M HCl. A 1 μl aliquot was transferred to 0.1 ml NAD cycling reagent and subjected to 30,000 cycles of amplification. α-Ketoglutarate standards were carried throughout all steps.

Ethanol: A 0.5 μl aliquot of an acid extract from cecal contents was added to 0.5 μl of a solution consisting of 5 mM Tris HCl (pH 8.1), 0.04% BSA, 0.1 mM NAD+, and 20 μg/ml yeast alcohol dehydrogenase (350 units/mg protein). Following a 60 min incubation at 24° C., 1 μl of 0.15M NaOH was added and the mixture heated for 20 min at 80° C. A 0.5 μl aliquot of this reaction mixture was transferred to 0.1 ml of NAD cycling reagent and amplified 5000-fold. Ethanol standards were carried throughout all steps.

Whole Genome Genotyping with Custom M. smithii Gene Chips

GeneChips were manufactured by Affymetrix (http://www.affymetrix.com), based on the sequence of the PS strain genome (see Table 13 for details of the GeneChip design). Duplicate cultures of M. smithii strains PS (ATCC 35061), F1 (DSMZ 2374), ALI (DSMZ 2375) and B181 (DSMZ 11975), were grown in 125 ml serum bottles as described above. Genomic DNA was prepared from each strain using the QIAGEN Genomic DNA Isolation kit: mutanolysin (Sigma; 2.5 U/mg wet wt. cell pellet) was added to facilitate lysis of the microbes. DNA (5-7 μg) was further purified by phenolchloroform extraction and then sheared by sonication to <200 bp, labeled with biotin (Enzo BioArray Terminal Labeling Kit), denatured at 95° C. for 5 min, and hybridized to replicate GeneChips using standard Affymetrix protocols (http://www.affymetrix.com). M. smithii genes represented on the GeneChip were called “Present” or “Absent” by DNA-Chip Analyzer v1.3 (dChip; www.biostat.harvard.edu/complab/dchip/) using modeled (PM/MM ratio) data.

### Statistical Analysis

Pairwise comparisons were made using unpaired Student's t-test. One-way ANOVA, followed by Tukey's post hoc multiple comparison test, was used to determine the statistical significance of differences observed between three groups.

### Development of PHAT (Pressurized Heated Anaerobic Tank) System

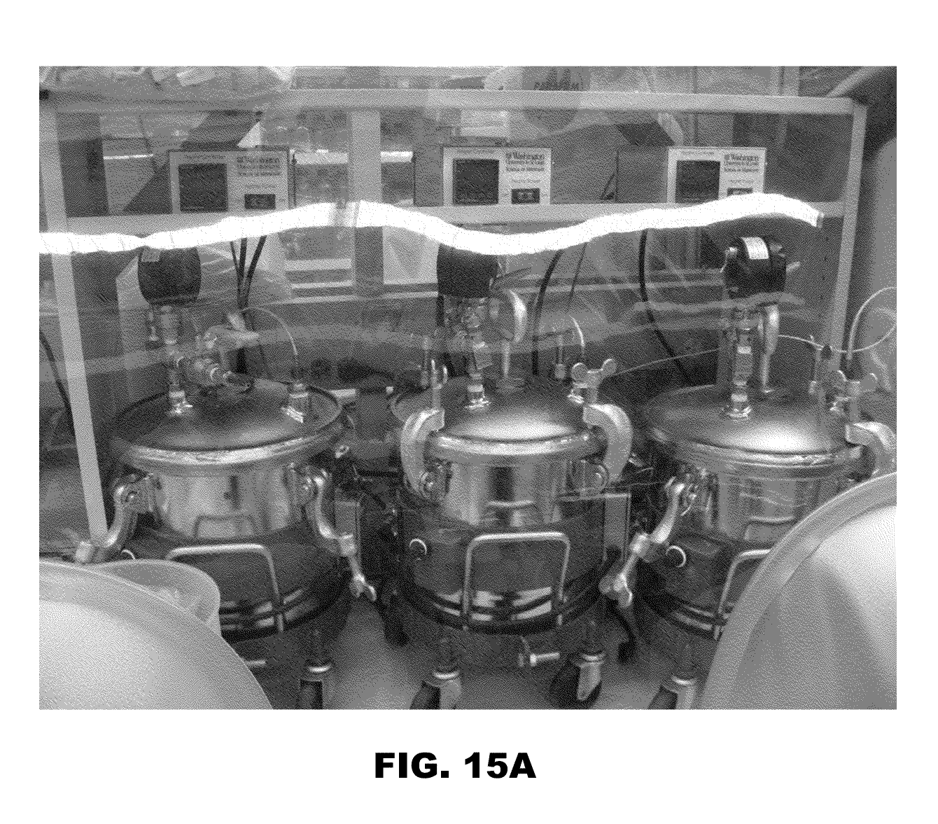

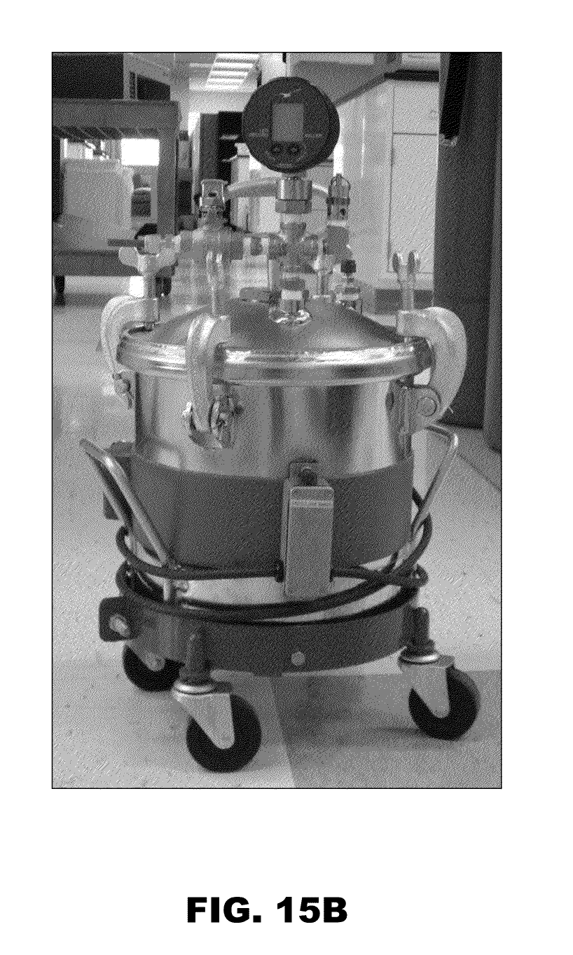

A system for culturing M. smithii in 96-well plate format was designed and constructed in the following manner (See FIG. 15). Three stainless steel paint canisters (Binks, 83S-210, 2 gallon size) were modified for incubation of plates at 37° C. in an oxygen-free gas mix of 20% CO₂/80% H₂ at a pressure of 30 psi, where all of these growth parameters can be monitored and recorded.

The canisters are heated using Electro-Flex Heat brand Pail Heaters controlled by a custom designed controller consisting of a 16A2120 temperature/process control (Love Controls), an RTD (resistance temperature detector) probe to measure internal tank temperature, and several safety features to prevent overheating or burns.

The system is pressurized with oxygen-free gas that has flowed through a custom-built oxygen scrub. Commercially available gas mixes used for culturing M. smithii contain trace levels of oxygen that would kill the organism: thus, the gas mixture must be passed through an oxygen scrub. This scrub consists of a glass tube filled with copper mesh that is heated to 350° C. with heating tape (HTS/Amptek Duo-Tape), controlled by a benchtop power controller (HTS/Amptek BT-Z). The oxygen scrub is covered with insulating tape and secured behind a heat resistant polyetherimide case. Pressure in each tank is measured and recorded with a digital manometer (LEO record, Omni Instruments).

The system is housed inside an anaerobic chamber (COY laboratories) to allow inspection and manipulation of cultures and plates without exposing M. smithii to oxygen. Each tank can house 30 standard volume 96-well plates, which can be analyzed inside the COY anaerobic chamber with a microplate reader (BioRad) that monitors growth by measuring optical density.

### Statin Susceptibility

Stock solutions (100×) of atorvastatin were prepared in methanol, pravastatin in ethanol, and rosuvastatin in DMSO (dimethyl sulfoxide) to concentrations of 100 mM, 10 mM and 1 mM. 1.5 μl of the stock solutions were added to wells in 96-well plates and transferred to the COY anaerobic chamber where they were kept for at least 24 hours to become anaerobic. 150 microliters of actively growing Methanobrevibacter smithii cultures were then added to each well (excluding medium+drug blanks) to bring the drug concentrations to 1 mM, 100 μM and 10 μM, respectively. The plates were incubated in the newly developed pressurized heated anaerobic tank system in a 4:1 mixture of oxygen-scrubbed H₂ and CO₂ at a pressure of 30 psi. Cultures grown in 1% ethanol, methanol and DMSO were used as controls. Growth was measured by determining optical density at 600 nm using the BioRad microplate reader (model 680).

Starting cultures of M. smithii strains [DSMZ 861 (PS), 2374 (F1), 2375 (ALI) and 11975 (B181)] were grown in 96 well plates in 150 μl volume/well of Methanobrevibacter complex medium (MBC) supplemented with 3 g/liter formate, 3 g/liter acetate, and 33 ml/liter of 2.5% Na₂S (added just before use). Each condition was tested in triplicate with the average measurement plotted.

### Example 1

### M. smithii Genome Description

The 1,853,160 base pair (bp) genome of the M. smithii type strain PS contains 1,795 predicted protein coding genes (Tables 1-4), 34 tRNAs, and two rRNA clusters. Some observations on the genome itself are as follows:

Elements that Affect Genome Evolution

The M. smithii PS genome contains multiple elements that can influence genome evolution, including 30 transposases, an integrated prophage (˜38 kb; MSM1640-92), eight insertion sequence (IS) elements, 16 genes involved in DNA repair, 9 restriction-modification (R-M) system subunits, and four predicted integrases (Table 4).

Several lytic phages have been reported to infect M. smithii, including a 69 kb linear phage known as PG that belongs to the ψM1-like viruses (Prangishvili et al. (2006) Virus Res 117:52-67), and another 35 kb phage (PMS11; Calendar (2005) The Bacteriophages). The PG phage is AT-rich, heavily nicked, and lytic (burst size, 30-90), with a latent period of 3-4 h (Bertani et al. (1985) EMBO Workshop on Molecular Genetics of Archaebacteria and the International Workshop on Biology and Biochemistry of Archaebacteria, pg. 398). BLAST comparisons of the 52 predicted genes in the integrated prophage of M. smithii PS against known phage genes revealed only a few homologs (Table 15). One of the prophage genes (MSM1691) encodes a pseudomurein endoisopeptidase (PeiW): this enzyme may function to cleave M. smithii's cell wall and contribute to autolysis, as related enzymes in a defective Methanothermobacter wolfeii prophage have been shown to do (Luo et al., FEMS Microbiology Letters 208:47-51). The specific ends of the prophage genome could not be identified, and further studies are needed to determine whether the prophage is active and lytic.

The eight insertion sequence (IS) elements in M. smithii's genome (Table 4) range in length from 137 bp (MSM1519) to 1013 bp (MSM0527) and all are ISM1 (family ISNCY) according to ISfinder (Siguier et al., (2006) Nucleic Acids Res 34:D32-6; http://www-is.biotoul.fr/). ISM1 is a mobile IS element (Hamilton and Reeve (1985) Molecular Genetics and Genomics 200:47-59). IS elements promote genome evolution and plasticity through recombination, gene loss and, potentially, lateral gene transfer (Brugger et al., (2002) FEMS Microbiol Lett 206:131-41).

### Transcriptional Regulation

M. smithii PS contains 60 predicted transcriptional regulators, including homologs of known nutrient sensors [e.g., a HypF family member (maturation of hydrogenases), a PhoU family member (phosphate metabolism), and a NikR family member (nickel)], plus five regulators of amino acid metabolism (Table 3). However, several GO categories related to environmental sensing and regulation (e.g., two-component systems; GO:0000160) are significantly depleted in its proteome compared to the proteomes of methanogens that live in terrestrial or aquatic environments (Table 6). In contrast, B. thetaiotaomicron, which uses complex, structurally diversified glycans as its principal nutrient source, possesses a large and diverse arsenal of nutrient sensors including 32 hybrid two-component systems plus 50 ECF-type sigma factors and 25 anti-sigma factors (Sonnenburg et al, (2006) PNAS 103:8834-9; Xu et al., (2003) Science 299:2074-6). This relative paucity of nutrient sensors may reflect the fact that M. smithii's niche is restricted, and its nutrient substrates are relatively small, readily diffusible molecules that may not require extensive machinery for their recognition.

### Bile Acid Detoxification

In humans, cholic and chenodeoxycholic acids are synthesized in the liver and during their enterohepatic circulation undergo transformation by the intestinal microbiota to an array of metabolites (Hylemon and Harder (1998) FEMS Microbiol Rev 22:475-88). Bile acids and their metabolites have microbicidal activity and a genetically engineered deficiency of the bile acid-activated nuclear receptor FXR leads to reduced bile acid pools and bacterial overgrowth (Inagaki et al., (2006) PNAS 103:3920-5). Both M. smithii and M. stadtmanae encode a sodium:bile acid symporter (MSM1078), a conjugated bile acid hydrolase (CBAH; MSM0986), a short chain dehydrogenase with homology to a 7α-hydroxysteroid dehydrogenase (MSM0021). This is consistent with in vitro studies of M. smithii that demonstrate it is not inhibited by 0.1% deoxycholic acid (Miller et al, (1982) Appl Environ Microbiol 43:227-32).

We compared the proteome of M. smithii with the proteomes of (i) Methanosphaera stadtmanae, a methanogenic Euryarchaeote that is a minor and inconsistent member of the human gut microbiota (Eckburg et al., (2005) Science 308:1635-38), (ii) nine ‘non-gut methanogens’ recovered from microbial communities in the environment, and (iii) these non-gut methanogens plus an additional 17 sequenced Archaea (‘all archaea’) (Table 5).

Compared to non-gut methanogens and/or all archaea, M. smithii and M. stadtmanae are significantly enriched (binomial test, p<0.01) for genes assigned to GO (gene ontology) categories involved in surface variation (e.g., cell wall organization and biogenesis, see below), defense (e.g., multi-drug efflux/transport), and processing of bacteria-derived metabolites (Tables 6 and 7).

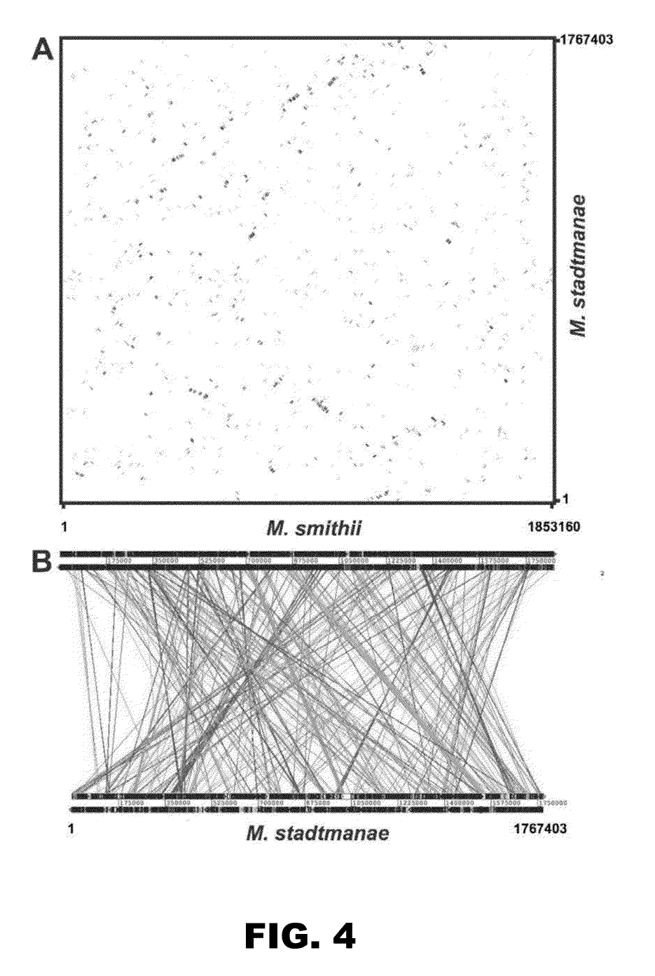

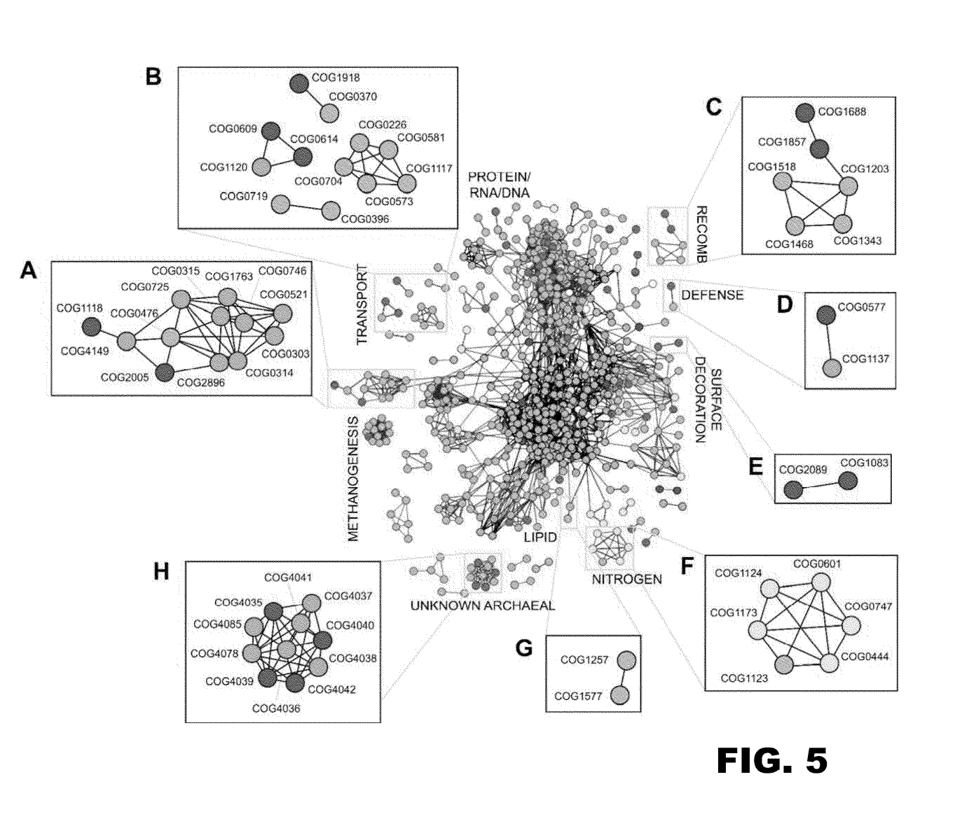

The M. smithii and M. stadtmanae genomes exhibit limited global synteny (FIG. 4) but share 968 proteins with mutual best BLAST hit e-values≦10-20 (46% of all M. smithii proteins; Table 8). A predicted interaction network of M. smithii clusters of orthologous groups (COGs) based on STRING, a database of predicted functional associations between proteins (von Mering et al., (2003) Nucleic Acids Res 31:258-61), shows that it contains more COGs for persistence, improved metabolic versatility, and machinery for genomic evolution compared to M. stadtmanae (FIG. 5 and Table 9).

### Cell Surface Variation

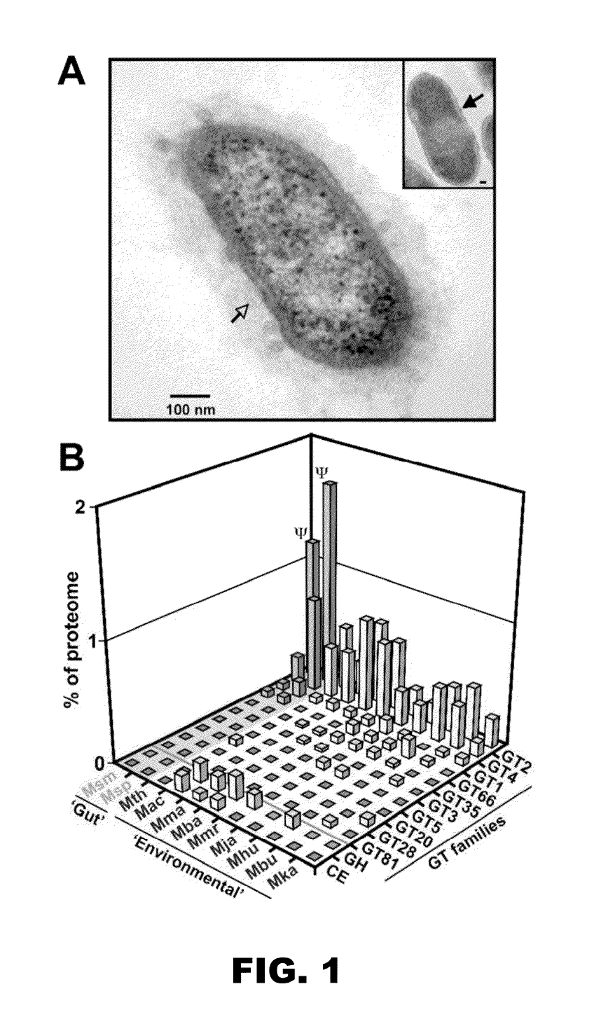

The ability to vary capsular polysaccharide surface structures in vivo by altering expression of glycosyltransferases (GTs) is a feature shared among sequenced bacterial species that are prominent in the distal human gut microbiota (Sonnenburg et al., (2005) Science 307:1955-59; Sonnenburg et al., (2006) PNAS 103:8834-39; Mazmanian et al., (2005) Cell 122:107-118; Coyne et al., (2005) Science 307:1778-81). Transmission EM studies of M. smithii harvested from gnotobiotic mice after a 14 day colonization revealed that it too has a prominent capsule (FIG. 1A). The proteomes of both human gut methanogens also contain an arsenal of GTs [26 in M. smithii and 31 in M. stadtmanae; see Table 10 for a complete list organized based on the Carbohydrate Active enZyme (CAZy) classification scheme (http://www.cazy.org; (Coutinho et al., (1999) Recent Advances in Carbohydrate Bioengineering)]. Unlike the sequenced Bacteroidetes, which possess large repertoires of glycoside hydrolases (GH) and carbohydrate esterases (CE) not represented in the human ‘glycobiome’, neither gut methanogen has any detectable GH or CE family members (FIG. 1B). Both M. smithii and M. stadtmanae dedicate a significantly larger proportion of their ‘glycobiome’ to GT2 family glycosyltransferases than any of the sequenced nongut associated methanogens (binomial test; p<0.00005; FIG. 1B). These GT2 family enzymes have diverse predicted activities, including synthesis of hyaluronan, a component of human glycosaminoglycans in the mucosal layer.

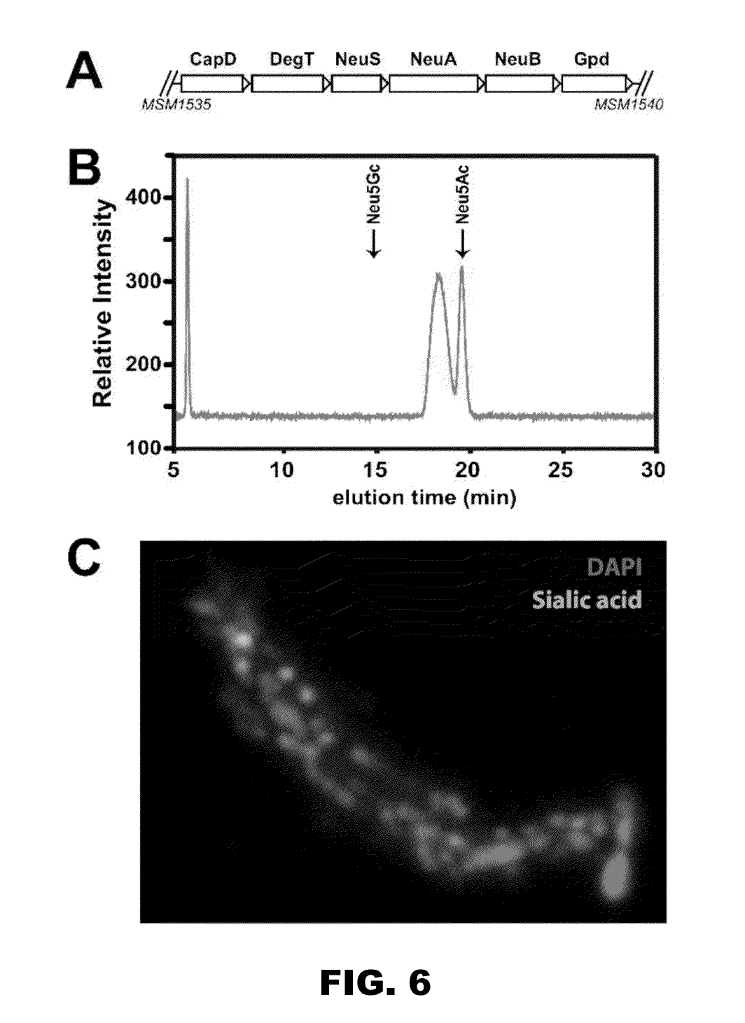

Sialic acids are a family of nine-carbon sugars that are abundantly represented in human mucus- and epithelial cell surface-associated glycans (Vimr et al., (2004) Microbiol Mol Biol Rev 68:132-53). N-acetylneuraminic acid (Neu5Ac) is the predominant type of sialic acid found in our species. Unique among sequenced archaea, M. smithii has a cluster of genes (MSM1535-1540) that encode all enzymes necessary for de novo synthesis of sialic acid from UDP-N-acetylglucosamine (i.e. UDP-GlcNAc epimerase, Neu5Ac synthase, CMP-Neu5Ac synthetase, and a putative polysialtransferase) (FIG. 1C). qRT-PCR assays of RNAs prepared from the cecal contents of 12-week-old gnotobiotic mice that had been colonized for 14d with the archaeon alone, or with B. thetaiotaomicron for 14d followed by addition of M. smithii for 14d (n=5-6 mice/treatment group) revealed that this cluster of genes is expressed in vivo at equivalent levels in mono- and co-colonized mice (n=5-6 animals/group; Table 11). Biochemical analysis of extracts prepared from cultured M. smithii, plus histochemical staining of the microbe with the sialic-acid specific lectin, Sambucus nigra 1 agglutinin (SNA), confirmed the presence of Neu5Ac (FIG. 6A-C). Taken together, our findings indicate that M. smithii has developed mechanisms to decorate its surface with carbohydrate moieties that mimic those encountered in the glycan landscape of its intestinal habitat.

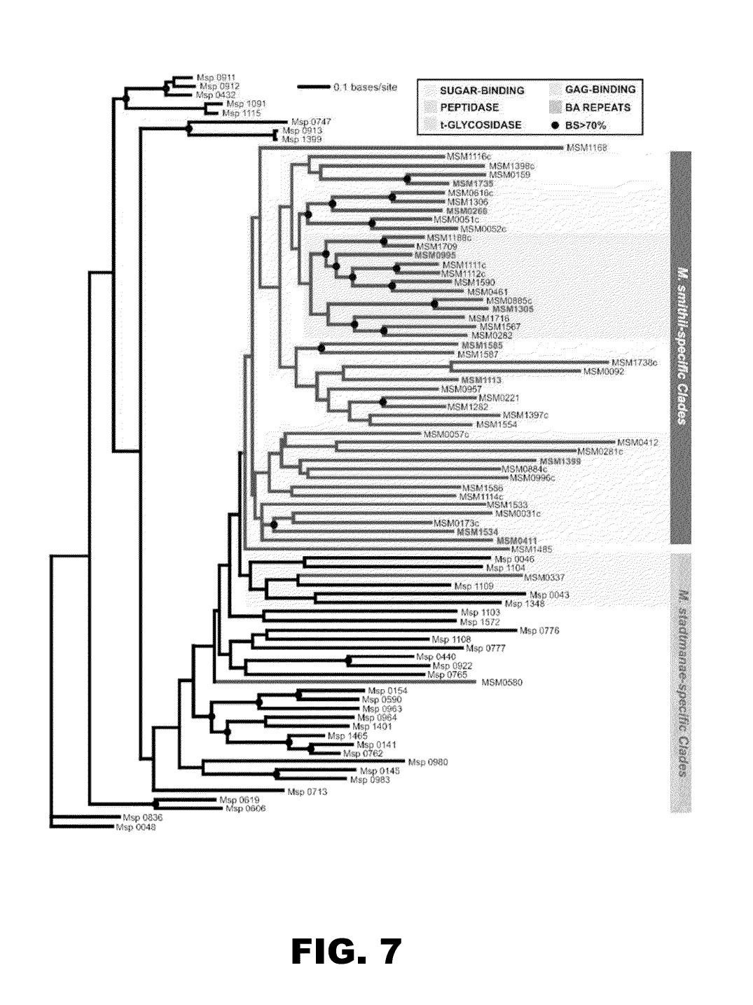

The genomes of both human gut methanogens also encode a novel class of predicted surface proteins that have features similar to bacterial adhesins (48 members in M. smithii and 37 in M. stadtmanae). A phylogenetic analysis indicated that each methanogen has a specific Glade of these Adhesin-Like Proteins (ALPs; FIG. 7A). A subset of the M. smithii ALPs has homology to pectin esterases (GO:0030599): this GO family, which is significantly enriched in this compared to other Archaea based on the binomial test (p<0.0005; Table 6), is associated with binding of chondroitin, a major component of mucosal glycosaminoglycans. Several other M. smithii ALPs have domains predicted to bind other sugar moieties (e.g. galactose-containing-glycans; FIG. 7A). Both methanogens also have ALPs with peptidase-like domains (see Table 12 for a complete list of InterPro domains).

We conducted qRT-PCR assays of cecal RNAs from the mono- and co-colonized gnotobiotic mice described above. The results revealed one ‘sugar-binding’ ALP (MSM1305) that was significantly upregulated in the presence of B. thetaiotaomicron, four that were suppressed (including one with a GAG binding domain), and two that exhibited no statistically significant alterations (FIG. 7B). Regulated expression of distinct subsets of ALPs may direct this methanogen to specific intestinal microhabitats where close association with saccharolytic bacterial partners could promote establishment and maintenance of syntrophic relationships: e.g., such intimate association is needed given the limited diffusion of H₂.

### Example 2

### Methanogenic and Non-Methanogenic Removal of Bacterial End-Products of Fermentation

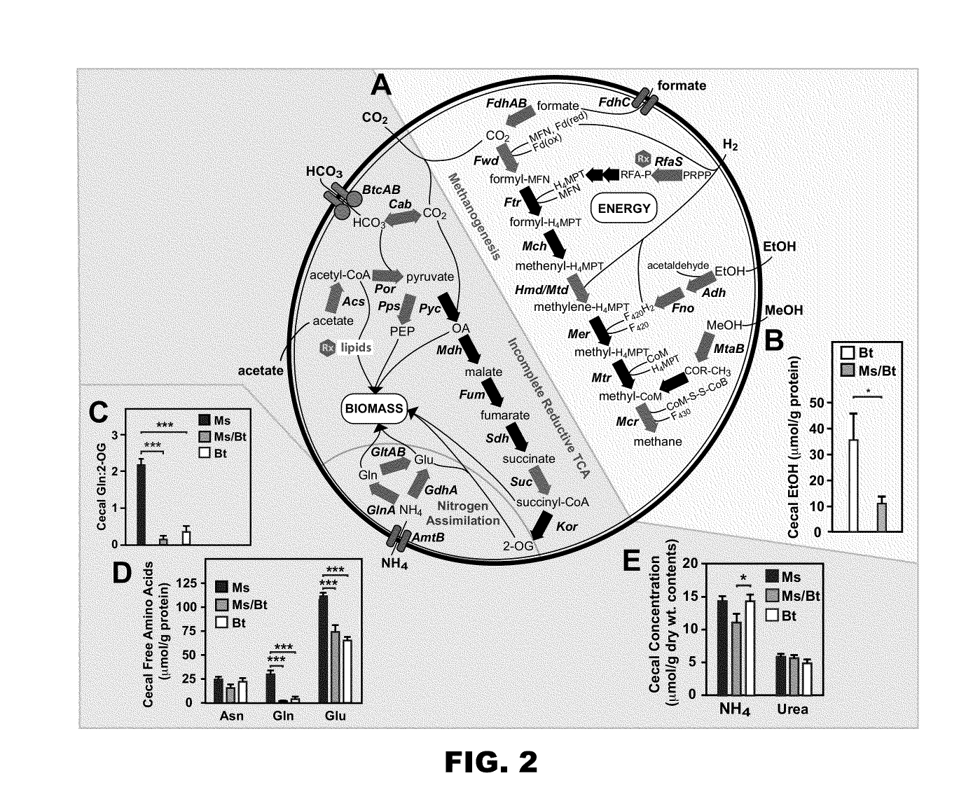

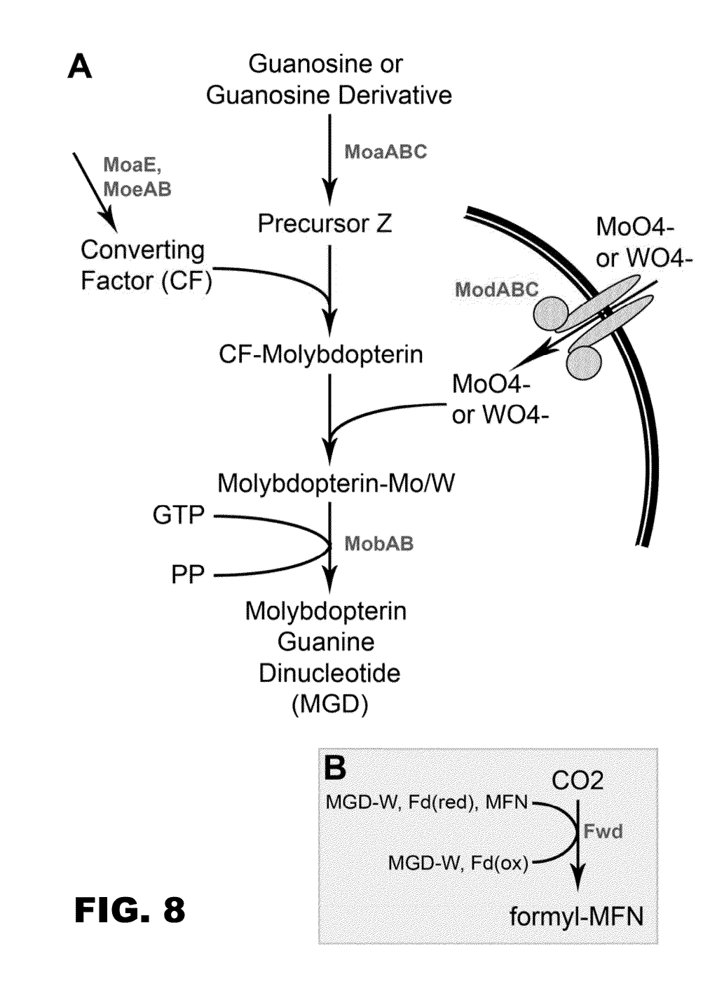

Compared to other sequenced non-gut associated methanogens, M. smithii has significant enrichment of genes involved in utilization of CO₂, H₂ and formate for methanogenesis (GO:0015948; Table 6). They include genes that encode proteins involved in synthesis of vitamin cofactors used by enzymes in the methanogenesis pathway [methyl group carriers (F₄₃₀ and corrinoids); riboflavin (precursor for F₄₃₀ biosynthesis); and coenzyme M synthase (involved in the terminal step of methanogenesis)] (see Table 7 for a list of these genes, and FIG. 2A for the metabolic pathways). M. smithii also has an intact pathway for molybdopterin biosynthesis to allow for CO₂ utilization (FIG. 8). qRT-PCR assays demonstrated that while key central methanogenesis enzymes are constitutively expressed in the presence or absence of B. thetaiotaomicron [see Fwd (tungsten formylmethanofuran dehydrogenase), Hmd (methylene-H₄ MPT dehydrogenase) and Mcr (methyl-CoM reductase)], ribofuranosylaminobenzene 5′-phosphate (RFA-P)-synthase (RfaS, MSM0848), an essential gene involved in methanopterin biosynthesis is significantly upregulated with co-colonization (see FIG. 2A and Table 11 for qRT-PCR results). M. smithii also upregulates a formate utilization gene cluster (FdhCAB; MSM1403-5) for methanogenic consumption of this B. thetaiotaomicron-produced metabolite (Samuel and Gordon (2006) PNAS 103:10011-10016).

Our previous qRT-PCR and mass spectrometry studies revealed that co-colonization increased B. thetaiotaomicron acetate production [acetate kinase (BT3963) 9-fold upregulated vs. B. thetaiotaomicron-mono-associated controls; P<0.0005; n=4-5 animals/group (Samuel and Gordon (2006) PNAS 103:10011-10016)]. Although acetate is not converted to methane by M. smithii (Miller et al., (1982) Appl. Environ. Microbiol. 43:227-32), we found that its proteome contains an ‘incomplete reductive TCA cycle’ that would allow it to assimilate acetate [Acs (acetyl-CoA synthase, MSM0330), Por (pyruvate:ferredoxin oxidoreductase, MSM0560), Pyc (pyruvate carboxylase, MSM0765), Mdh (malate dehydrogenase, MSM1040), Fum (fumarate hydratase, MSM0477, MSM0563, MSM0769, MSM0929), Sdh (succinate dehydrogenase, MSM1258), Suc (succinyl-CoA synthetase, MSMO228, MSM0924), and Kor (2-oxoglutarate synthase, MSM0925-8) in FIG. 2A]. qRT-PCR assays disclosed that co-colonization upregulated two important M. smithii genes associated with this pathway that participate in acetate assimilation: Por (pyruvate:ferredoxin oxidoreductase) as well as Cab (carbonic anhydrase, MSM0654), which converts CO₂ to bicarbonate, the substrate for Por (FIG. 2B).

M. smithii also possesses enzymes that in other methanogens facilitate utilization of two other products of bacterial fermentation, methanol and ethanol (Fricke et al, J Bacteriol 188:642-58; Berk et al., (1997) Arch Microbiol 168:396-402). qRT-PCR assays showed that co-colonization significantly increased expression of a methanol:cobalamin methyltransferase (MtaB, MSM0515), an NADP-dependent alcohol dehydrogenase (Adh, MSM1381), and an F₄₂₀-dependent NADP reductase (Fno, MSM0049) [2.4±0.3, 2.3±0.4 and 3.7±0.4 fold vs. mono-associated controls, respectively; p<0.01; see FIG. 2A for pathway information and FIG. 2C for qRT-PCR results]. Follow-up biochemical studies confirmed a significant decrease in ethanol levels in the ceca of co-colonized mice [35±6 μmol/g total protein in cecal contents versus 11±2 μmol/g and 12±2 μmol/g in B. thetaiotaomicron and M. smithii mono-associated animals respectively; p<0.05; FIG. 2D]. Expression of B. thetaiotaomicron's alcohol dehydrogenases (BT4512 and BT0535) is not altered by co-colonization (Samuel and Gordon (2006) PNAS 103:10011-10016), indicating that the reduction in cecal ethanol levels observed in co-colonized mice is not due to diminished bacterial production but rather to increased archaeal consumption.

Collectively, these findings indicate that M. smithii supports methanogenic and non-methanogenic removal of diverse bacterial end-products of fermentation: this capacity may endow it with a great flexibility to form syntrophic relationships with a broad range of bacterial members of the distal human gut microbiota.

### Example 3

### M. smithii Utilization of Ammonia as a Primary Nitrogen Source

Subject metabolism of amino acids by glutaminases associated with the intestinal mucosa (Wallace (1996) J Nutr 126:1326 S), or deamination of amino acids during bacterial degradation of dietary proteins yields ammonia (Cabello et al., (2004) Microbiology 150:3527-46). The M. smithii proteome contains a transporter for ammonium (AmtB; MSMO234) plus two routes for its assimilation: (i) the ATP-utilizing glutamine synthetase-glutamate synthase pathway which has a high affinity for ammonium and thus is advantageous under nitrogen-limited conditions; and (ii) the ATP-independent glutamate dehydrogenase pathway which has a lower affinity for ammonium (Dumitru et al., (2003) Appl. Environ. Microbiol. 69:7236-41).

Microanalytic biochemical assays revealed a ratio of glutamine to 2-oxoglutarate concentration that was 32-fold lower in the ceca of co-colonized gnotobiotic mice compared to animals colonized with M. smithii alone, and 5-fold lower compared to B. thetaiotaomicron mono-associated subjects (p<0.0001; FIG. 2E). In addition, levels of several polar amino acids were also significantly reduced in mice with the saccharolytic bacterium and methanogen (FIG. 2F), providing additional evidence for a nitrogen-limited gut environment. qRT-PCR analyses established that many of the key M. smithii genes involved in ammonia assimilation are upregulated with co-colonization, particularly those in the high affinity glutamine synthetase-glutamate synthase pathway [GInA (glutamine synthetase, MSM1418); GltA/GltB (two subunits of glutamate synthase, MSM0027, MSM0368); FIG. 2A,G]. GeneChip analysis of the transcriptional responses of B. thetaiotaomicron to co-colonization with M. smithii indicated that it also upregulates a high affinity glutamine synthase [BT4339; 2.4-fold vs. B. thetaiotaomicron monoassociated mice; n=4-5 mice/group; p<0.001; (Samuel et al., (2006) PNAS 103:10011-10016)]. This prioritization of ammonium assimilation by B. thetaiotaomicron and M. smithii is accompanied by a decrease in cecal ammonium levels in co-colonized subjects (11.1±1.3 pmol/g dry weight of cecal contents vs. 14.4±0.6 in M. smithii- and 14.3±0.9 in B. thetaiotaomicron-monoassociated animals; n=5-15/group; p<0.05; FIG. 2H). Together, these studies indicate that ammonium provides a key source of nitrogen for M. smithii when it exists in isolation in the gut of gnotobiotic mice, and that it must compete with B. thetaiotaomicron for this nutrient resource.

### Example 4

### Considering Targets for Development of Anti-M. smithii Agents

Manipulation of the representation of M. smithii in our gut microbiota could provide a novel means for treating obesity. Functional genomics studies in gnotobiotic mice illustrate one way to approach the issue. For example, inhibitors exist for several M. smithii enzymes. A class of N-substituted derivatives of para-aminobenzoic acid (pABA) interfere with methanogenesis by competitively inhibiting ribofuranosylaminobenzene 5′-phosphate synthase [RfaS; MSM0848; (Dumitru et al., (2003) Appl. Environ. Microbiol. 69:7236-41)]. As noted above, this enzyme, which participates in the first committed step in synthesis of methanopterin, is upregulated with co-colonization (4.6±0.9 fold versus mono-associated controls; p<0.01; FIG. 2A).

Archaeal membrane lipids, unlike bacterial lipids, contain ether-linkages. A key enzyme in the biosynthesis of archaeal lipids is hydroxymethylglutaryl (HMG)-CoA reductase (MSMO227), which catalyzes the formation of mevalonate, a precursor for membrane (isoprenoid) biosynthesis (23). HMG-CoA reductase inhibitors (statins) inhibit growth of Methanobrevibacter species in vitro (23). qRT-PCR revealed that MSMO227 is expressed at high levels in vivo in the presence or absence of B. thetaiotaomicron (P>0.05; Table 11).

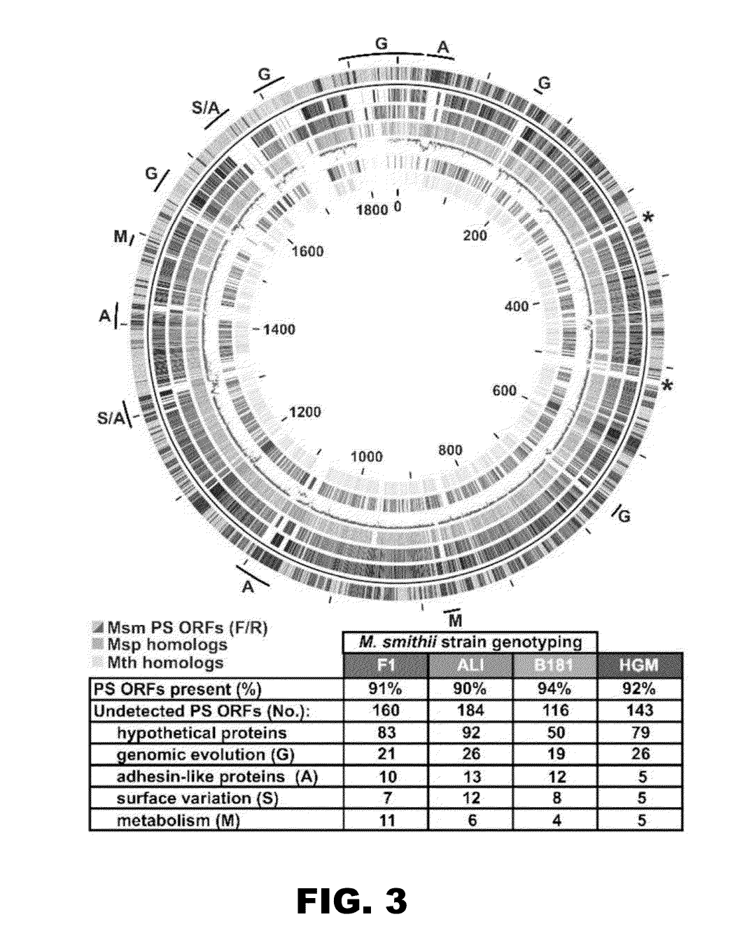

We designed a custom GeneChip containing probesets directed against 99.1% of M. smithii's 1795 known and predicted protein-coding genes (see Table 12 for details). This GeneChip was used to perform whole genome genotyping of M. smithii PS (control) plus three other strains recovered from the feces of healthy humans: F1 (DSMZ 2374), ALI (DSMZ 2375) and B181 (DSMZ 11975). Replicate hybridizations indicated that 100% of the open reading frames (ORFs) represented on the GeneChip were detected in M. smithii PS, while 90-94% were detected in the other strains, including the potential drug targets mentioned above (Table 2 and FIG. 3). Approximately 50% of the undetectable ORFs in each strain encode hypothetical proteins. The other undetectable genes are involved in genome evolution [e.g., recombinases, transposases, IS elements, and type II restriction modification (R-M) systems], or are components of a putative archaeal prophage in strain PS, or are related to surface variation, including several ALPs (e.g., MSM0057 and MSM1585-90; FIG. 7). Strains F1 and ALI also appear to lack redundant gene clusters encoding subunits of formate dehydrogenase (MSM1462-3) and methyl-CoM reductase (MSM0902-3) that are found in the PS strain (the latter cluster is also undetectable in strain B181). In addition, the only methanol utilization cluster present in the PS strain (MSM1515-8) was not detectable in strain F1 (Table 2).

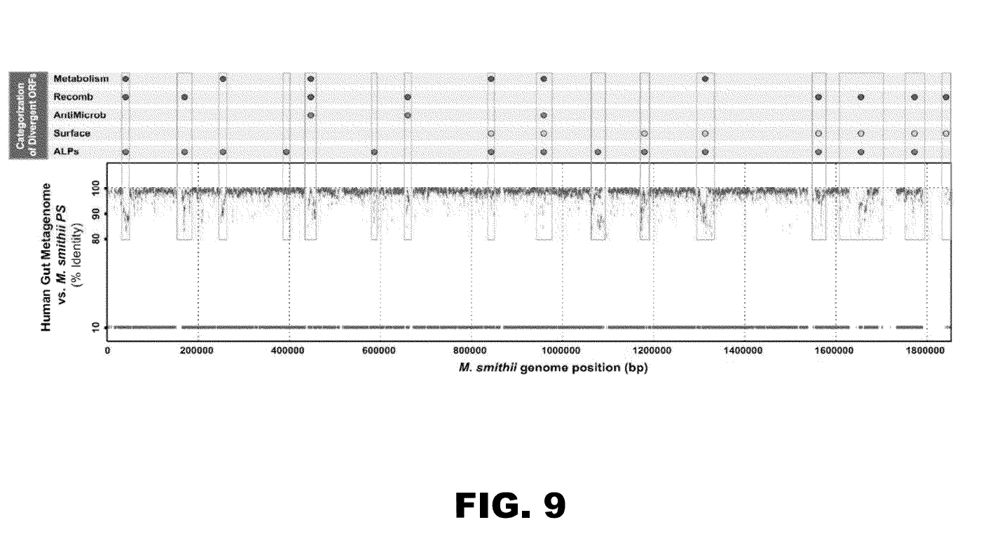

To further assess the degree of nucleotide sequence divergence among M. smithii strains, we compared the sequenced PS type strain to a 78 Mb metagenomic dataset generated from the aggregate fecal microbial community genome (microbiome) of two healthy humans (Gill et al., (2006) Science 312:1355-59). Their sequenced microbiomes contained 92% of the ORFs in the type strain (Table 2), including the potential drug targets described above. Several R-M system gene clusters (MSM0157-8, MSM1743, MSM1746-7), a number of transposases, a DNA repair gene cluster (MSM0689-95), and all ORFs in the prophage were not evident in the two microbiomes. Sequence divergence was also observed in 33 of the 48 ALP genes plus two ‘surface variation’ gene clusters (MSM1289-1398 and MSM1590-1616) that encode 11 glycosyltransferases and 9 proteins involved in pseudomurein cell wall biosynthesis (FIG. 9). A redundant methyl-CoM reductase cluster (MSM0902-3), an F₄₂₀-dependent NADP oxidoreductase (MSM0049) involved in consumption of bacteria-derived ethanol, and two subunits of the bicarbonate ABC transporter (MSM0990-1; carbon utilization) exhibited heterogeneity in the M. smithii populations present in the gut microbiota of these two adults (Table 2 and FIG. 9).

### Example 5

### Effect of HMG-CoA Reductase Inhibitors Administration

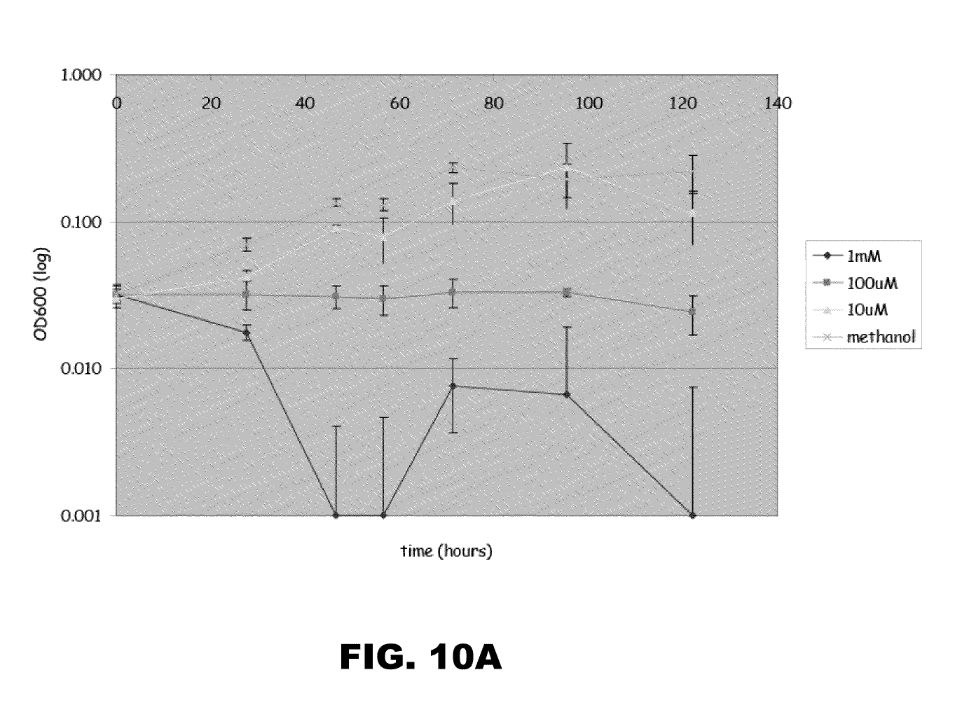

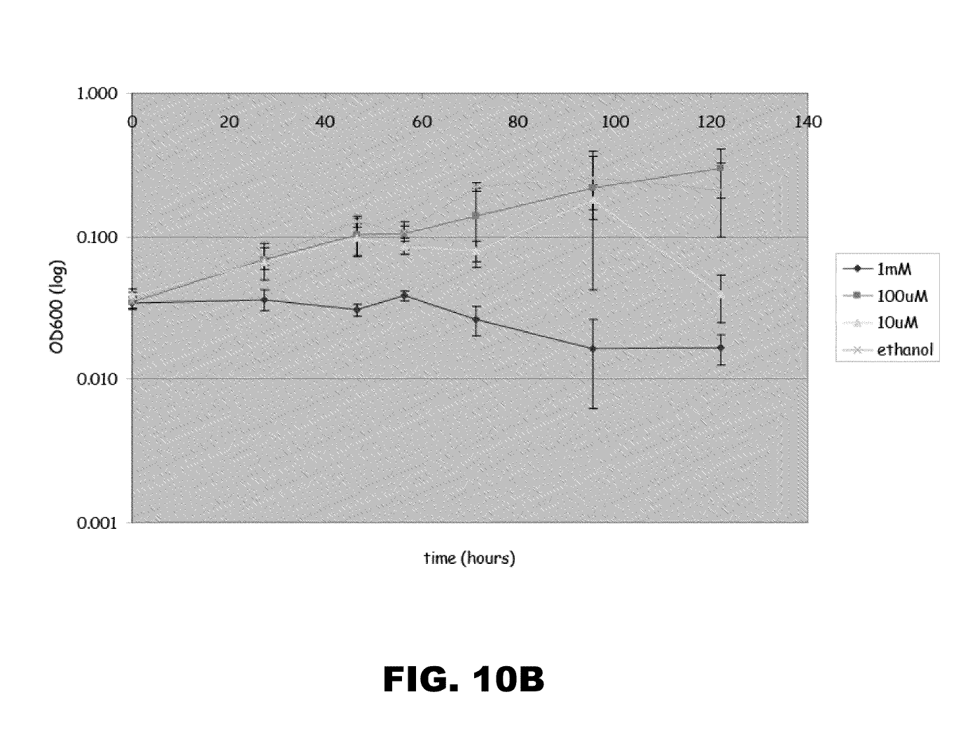

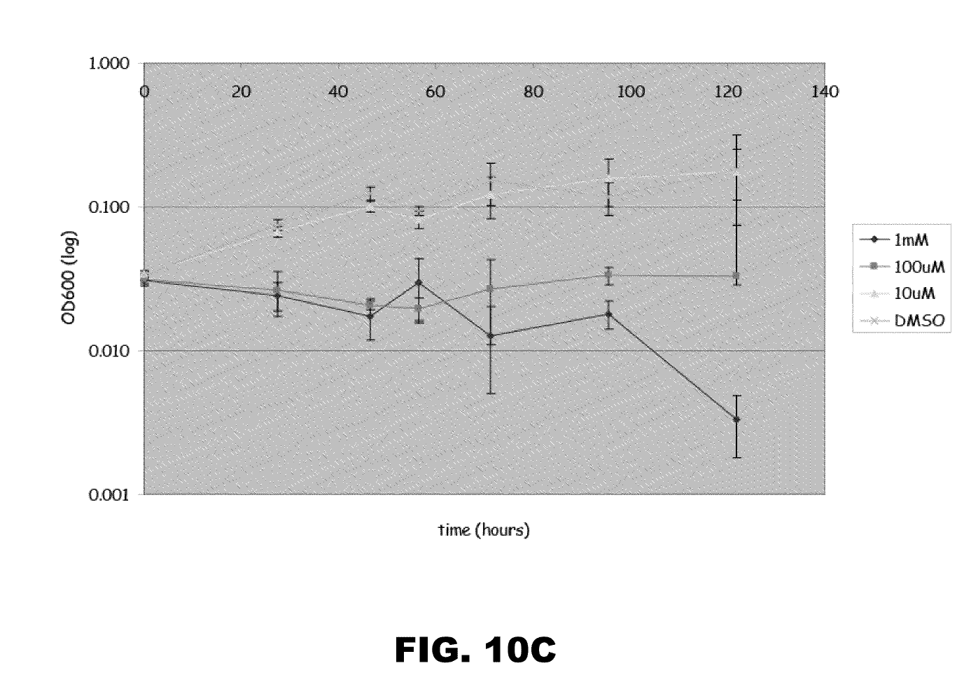

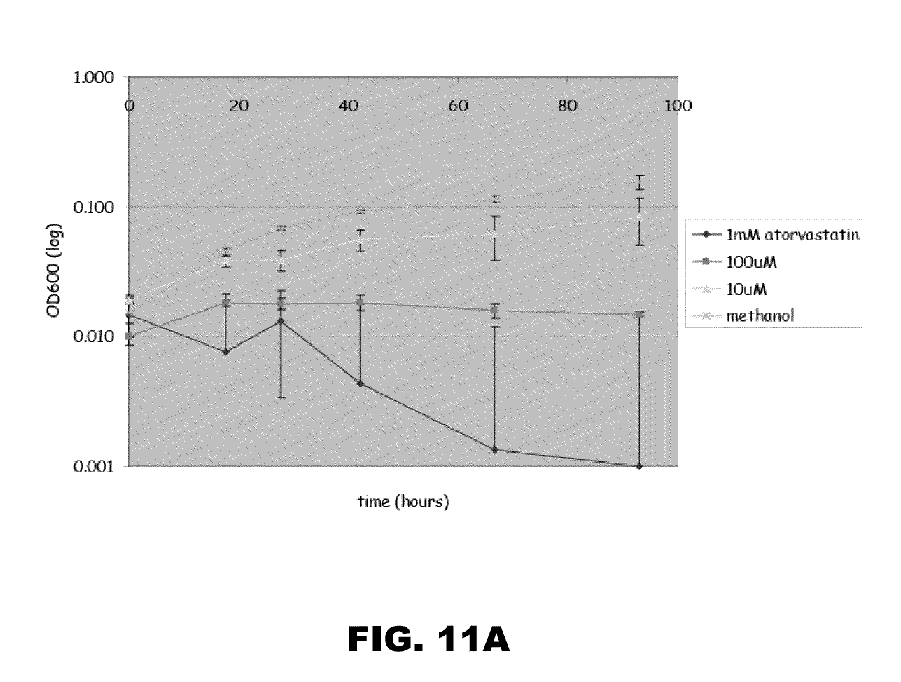

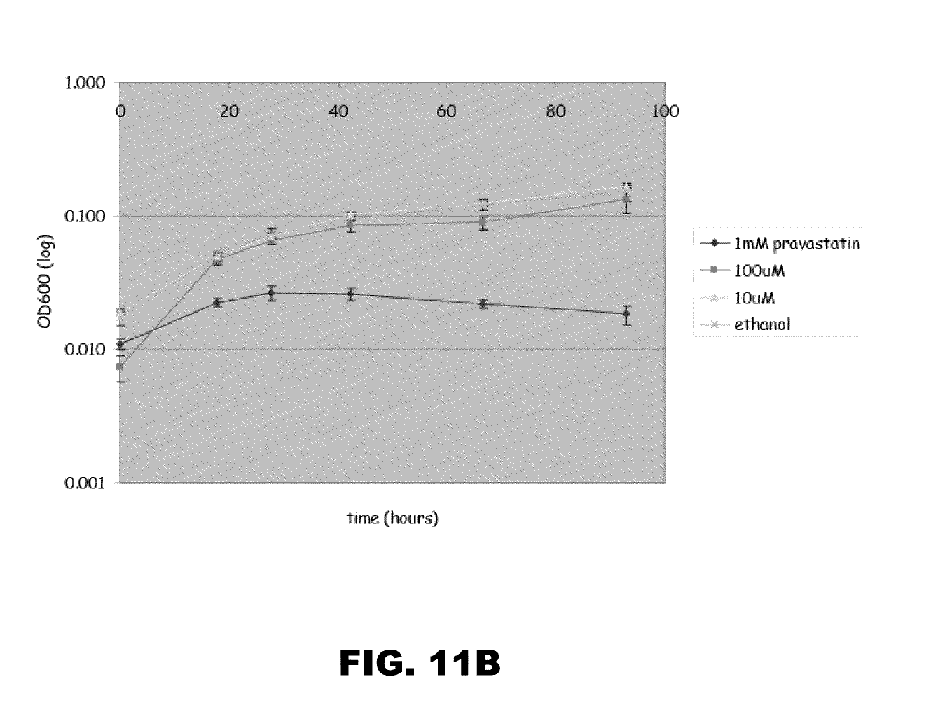

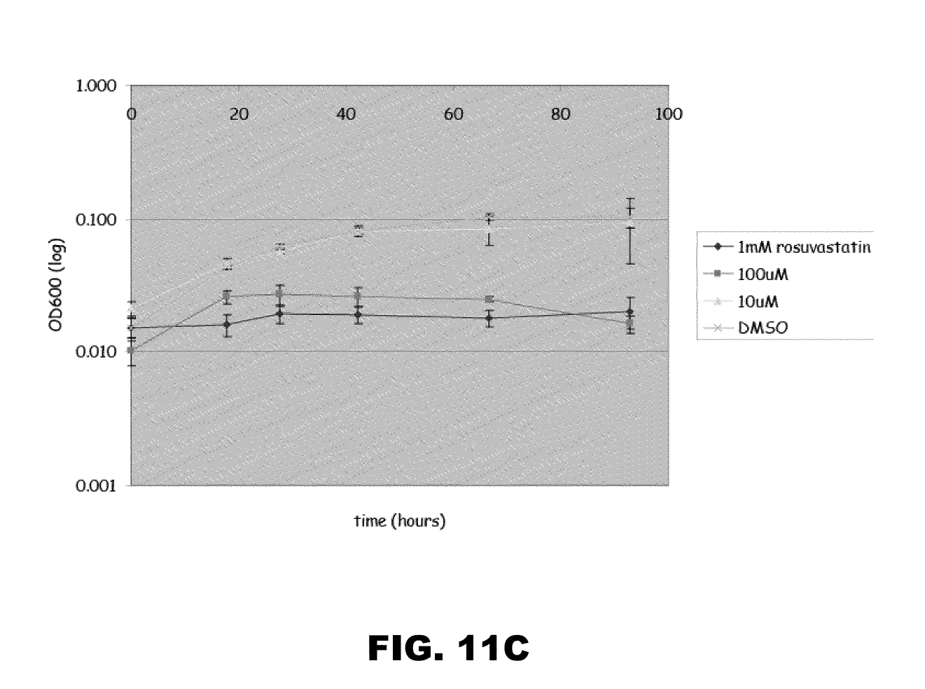

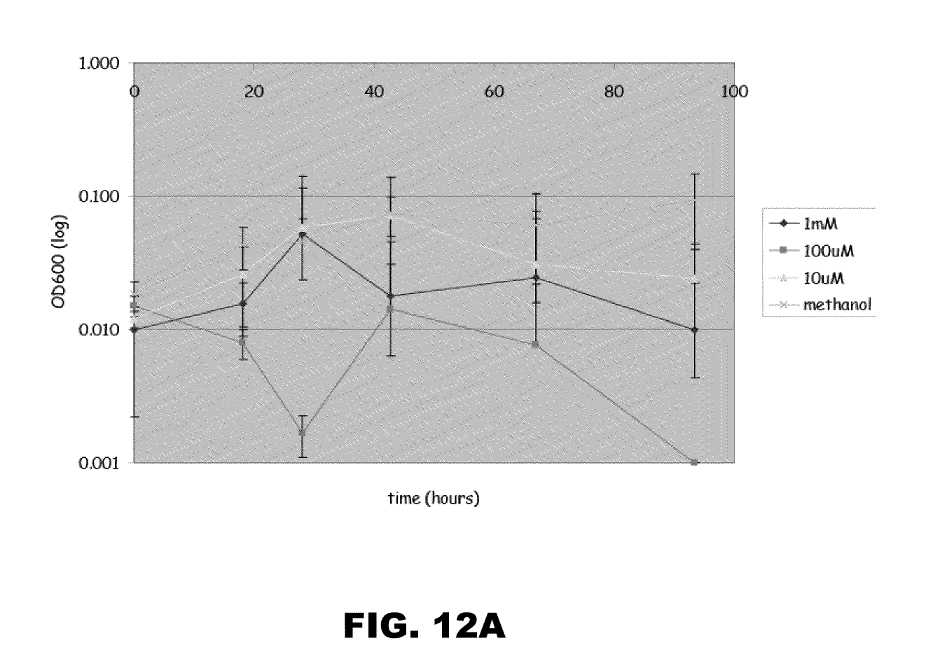

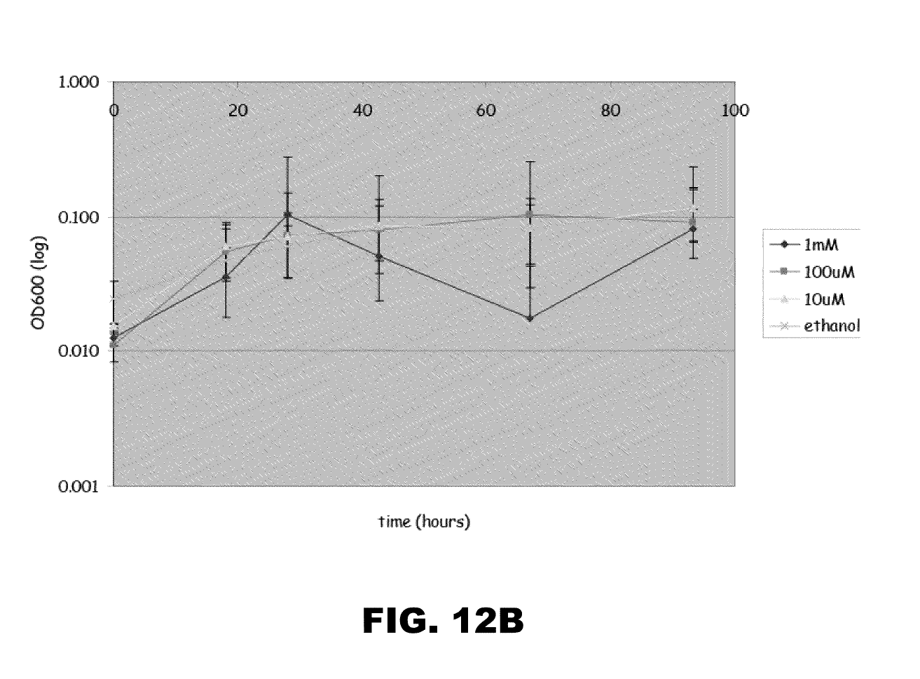

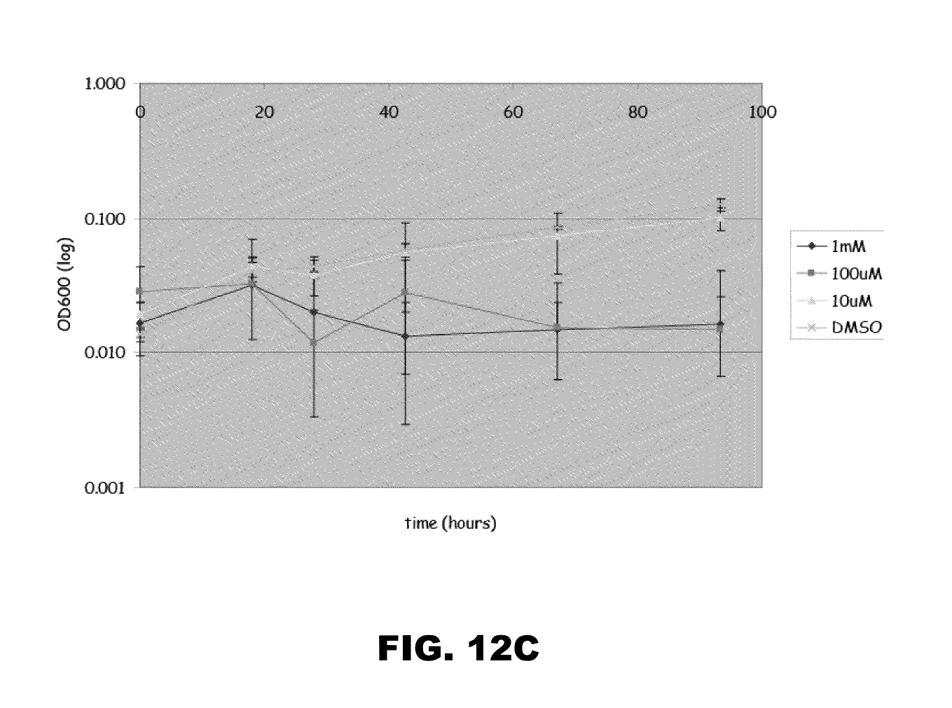

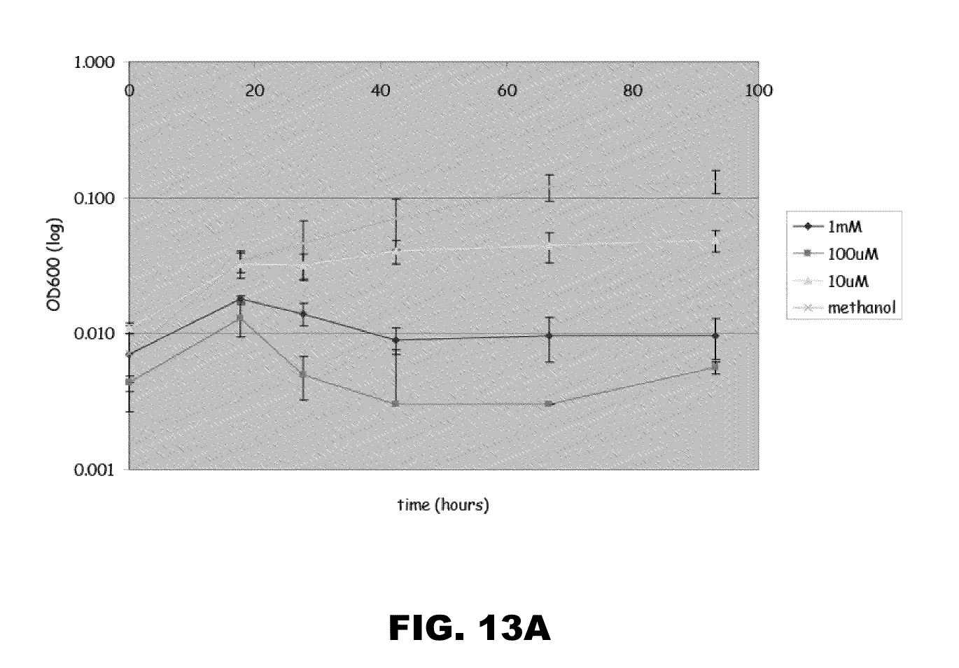

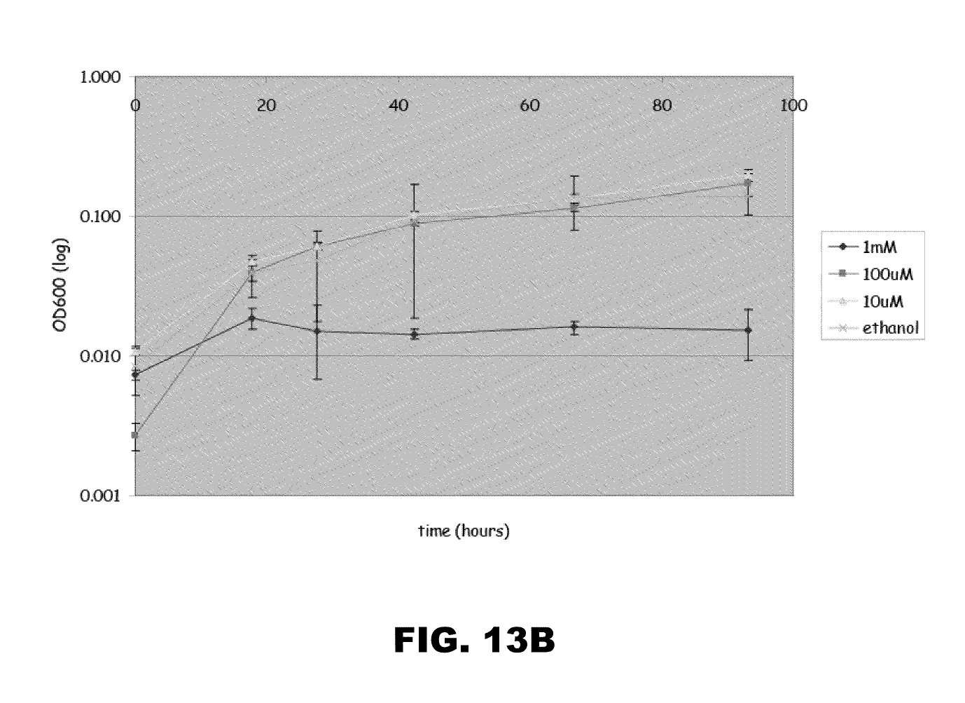

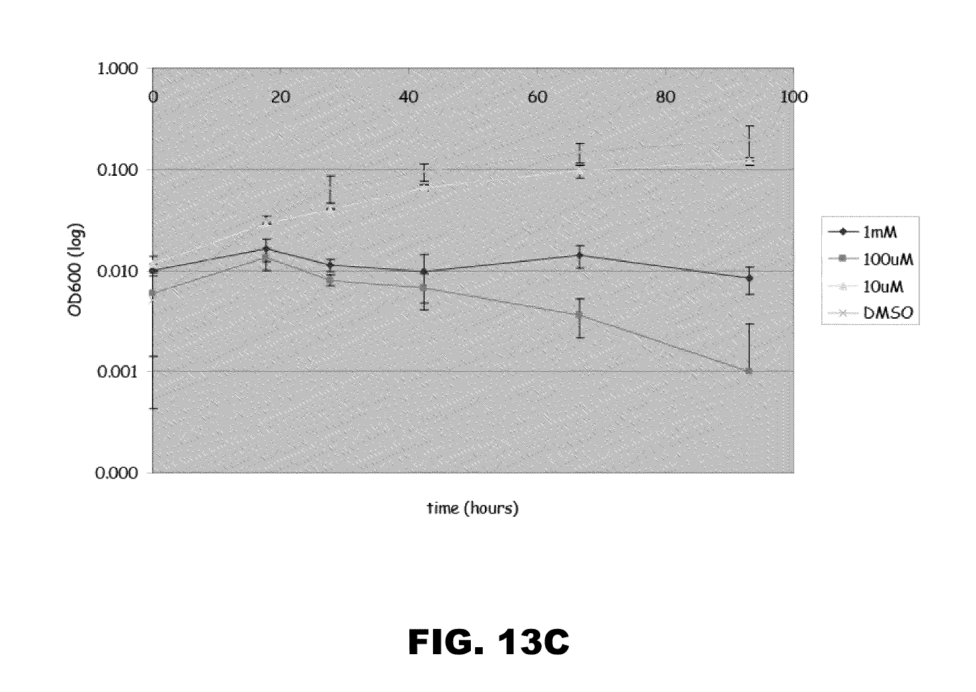

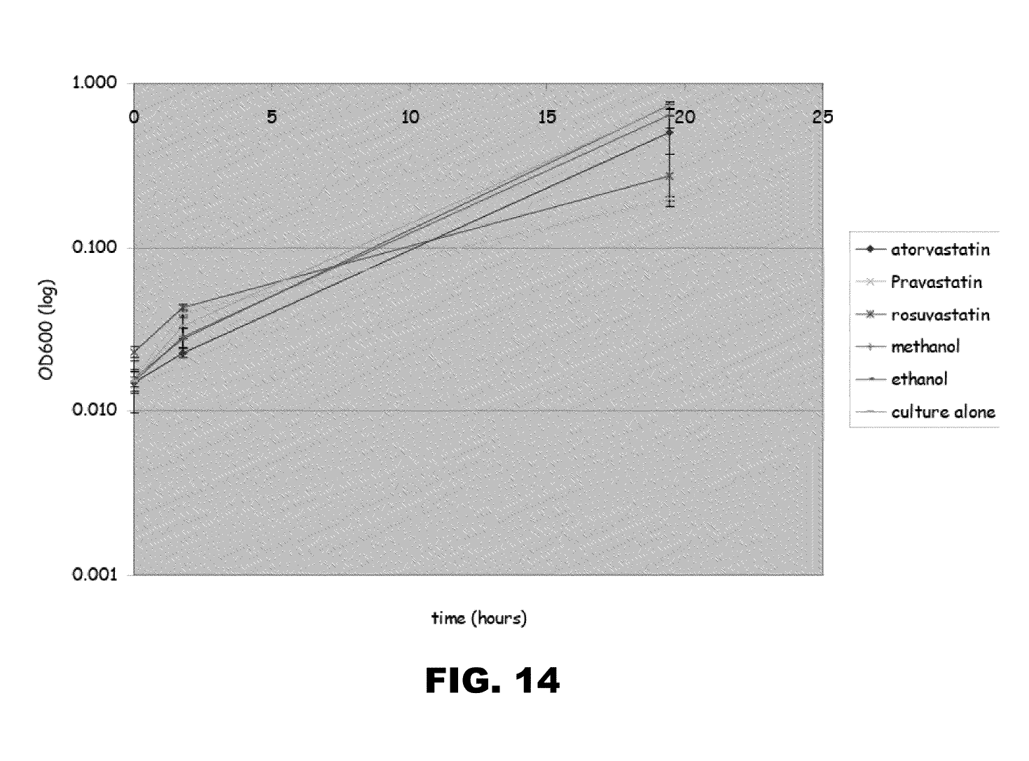

The PHAT system was used to culture 4 strains of M. smithii (DSMZ 861 (PS), 2374 (F1), 2375 (ALI) and 11975 (B181)) in 96-well plate format, and to test their sensitivities to various HMG-CoA reductase inhibitors. Preliminary results indicate that atorvastatin (Lipitor®), pravastatin (Pravachol®) and rosuvastatin (Crestor®) inhibit all strains tested at concentrations of 1 millimolar. Atorvastatin and rosuvastatin also inhibit all strains at 100 micromolar concentrations (FIG. 10-13; Tables 16-19). None of these three statins had any affect on the growth of a dominant human gut-associated saccharolytic bacterium, Bacteroides thetaiotaomicron (FIG. 14).

TABLE A

| | | | | |
|---|---|---|---|---|
| MSM0001 | MSM0002 | MSM0003 | MSM0004 | MSM0005 |
| MSM0006 | MSM0007 | MSM0008 | MSM0009 | MSM0010 |
| MSM0011 | MSM0012 | MSM0013 | MSM0014 | MSM0015 |
| MSM0016 | MSM0017 | MSM0018 | MSM0019 | MSM0020 |
| MSM0021 | MSM0022 | MSM0023 | MSM0024 | MSM0025 |
| MSM0026 | MSM0027 | MSM0028 | MSM0029 | MSM0030 |
| MSM0031 | MSM0032 | MSM0033 | MSM0034 | MSM0035 |
| MSM0036 | MSM0037 | MSM0038 | MSM0039 | MSM0040 |
| MSM0041 | MSM0042 | MSM0043 | MSM0044 | MSM0045 |
| MSM0046 | MSM0047 | MSM0048 | MSM0049 | MSM0050 |
| MSM0051 | MSM0052 | MSM0053 | MSM0054 | MSM0055 |
| MSM0056 | MSM0057 | MSM0058 | MSM0059 | MSM0060 |
| MSM0061 | MSM0062 | MSM0063 | MSM0064 | MSM0065 |
| MSM0066 | MSM0067 | MSM0068 | MSM0069 | MSM0070 |
| MSM0071 | MSM0072 | MSM0073 | MSM0074 | MSM0075 |
| MSM0076 | MSM0077 | MSM0078 | MSM0079 | MSM0080 |
| MSM0081 | MSM0082 | MSM0083 | MSM0084 | MSM0085 |
| MSM0086 | MSM0087 | MSM0088 | MSM0089 | MSM0090 |
| MSM0091 | MSM0092 | MSM0093 | MSM0094 | MSM0095 |
| MSM0096 | MSM0097 | MSM0098 | MSM0099 | MSM0100 |
| MSM0101 | MSM0102 | MSM0103 | MSM0104 | MSM0105 |
| MSM0106 | MSM0107 | MSM0108 | MSM0109 | MSM0110 |
| MSM0111 | MSM0112 | MSM0113 | MSM0114 | MSM0115 |
| MSM0116 | MSM0117 | MSM0118 | MSM0119 | MSM0120 |
| MSM0121 | MSM0122 | MSM0123 | MSM0124 | MSM0125 |
| MSM0126 | MSM0127 | MSM0128 | MSM0129 | MSM0130 |
| MSM0131 | MSM0132 | MSM0133 | MSM0134 | MSM0135 |
| MSM0136 | MSM0137 | MSM0138 | MSM0139 | MSM0140 |
| MSM0141 | MSM0142 | MSM0143 | MSM0144 | MSM0145 |
| MSM0146 | MSM0147 | MSM0148 | MSM0149 | MSM0150 |
| MSM0151 | MSM0152 | MSM0153 | MSM0154 | MSM0155 |
| MSM0156 | MSM0157 | MSM0158 | MSM0159 | MSM0160 |
| MSM0161 | MSM0162 | MSM0163 | MSM0164 | MSM0165 |
| MSM0166 | MSM0167 | MSM0168 | MSM0169 | MSM0170 |
| MSM0171 | MSM0172 | MSM0173 | MSM0174 | MSM0175 |
| MSM0176 | MSM0177 | MSM0178 | MSM0179 | MSM0180 |
| MSM0181 | MSM0182 | MSM0183 | MSM0184 | MSM0185 |
| MSM0186 | MSM0187 | MSM0188 | MSM0189 | MSM0190 |
| MSM0191 | MSM0192 | MSM0193 | MSM0194 | MSM0195 |
| MSM0196 | MSM0197 | MSM0198 | MSM0199 | MSM0200 |
| MSM0201 | MSM0202 | MSM0203 | MSM0204 | MSM0205 |
| MSM0206 | MSM0207 | MSM0208 | MSM0209 | MSM0210 |
| MSM0211 | MSM0212 | MSM0213 | MSM0214 | MSM0215 |
| MSM0216 | MSM0217 | MSM0218 | MSM0219 | MSM0220 |
| MSM0221 | MSM0222 | MSM0223 | MSM0224 | MSM0225 |
| MSM0226 | MSM0227 | MSM0228 | MSM0229 | MSM0230 |
| MSM0231 | MSM0232 | MSM0233 | MSM0234 | MSM0235 |
| MSM0236 | MSM0237 | MSM0238 | MSM0239 | MSM0240 |
| MSM0241 | MSM0242 | MSM0243 | MSM0244 | MSM0245 |
| MSM0246 | MSM0247 | MSM0248 | MSM0249 | MSM0250 |
| MSM0251 | MSM0252 | MSM0253 | MSM0254 | MSM0255 |
| MSM0256 | MSM0257 | MSM0258 | MSM0259 | MSM0260 |
| MSM0261 | MSM0262 | MSM0263 | MSM0264 | MSM0265 |
| MSM0266 | MSM0267 | MSM0268 | MSM0269 | MSM0270 |
| MSM0271 | MSM0272 | MSM0273 | MSM0274 | MSM0275 |
| MSM0276 | MSM0277 | MSM0278 | MSM0279 | MSM0280 |
| MSM0281 | MSM0282 | MSM0283 | MSM0284 | MSM0285 |
| MSM0286 | MSM0287 | MSM0288 | MSM0289 | MSM0290 |
| MSM0291 | MSM0292 | MSM0293 | MSM0294 | MSM0295 |
| MSM0296 | MSM0297 | MSM0298 | MSM0299 | MSM0300 |
| MSM0301 | MSM0302 | MSM0303 | MSM0304 | MSM0305 |
| MSM0306 | MSM0307 | MSM0308 | MSM0309 | MSM0310 |
| MSM0311 | MSM0312 | MSM0313 | MSM0314 | MSM0315 |
| MSM0316 | MSM0317 | MSM0318 | MSM0319 | MSM0320 |
| MSM0321 | MSM0322 | MSM0323 | MSM0324 | MSM0325 |
| MSM0326 | MSM0327 | MSM0328 | MSM0329 | MSM0330 |
| MSM0331 | MSM0332 | MSM0333 | MSM0334 | MSM0335 |
| MSM0336 | MSM0337 | MSM0338 | MSM0339 | MSM0340 |
| MSM0341 | MSM0342 | MSM0343 | MSM0344 | MSM0345 |
| MSM0346 | MSM0347 | MSM0348 | MSM0349 | MSM0350 |
| MSM0351 | MSM0352 | MSM0353 | MSM0354 | MSM0355 |
| MSM0356 | MSM0357 | MSM0358 | MSM0359 | MSM0360 |
| MSM0361 | MSM0362 | MSM0363 | MSM0364 | MSM0365 |
| MSM0366 | MSM0367 | MSM0368 | MSM0369 | MSM0370 |
| MSM0371 | MSM0372 | MSM0373 | MSM0374 | MSM0375 |
| MSM0376 | MSM0377 | MSM0378 | MSM0379 | MSM0380 |
| MSM0381 | MSM0382 | MSM0383 | MSM0384 | MSM0385 |
| MSM0386 | MSM0387 | MSM0388 | MSM0389 | MSM0390 |
| MSM0391 | MSM0392 | MSM0393 | MSM0394 | MSM0395 |
| MSM0396 | MSM0397 | MSM0398 | MSM0399 | MSM0400 |
| MSM0401 | MSM0402 | MSM0403 | MSM0404 | MSM0405 |
| MSM0406 | MSM0407 | MSM0408 | MSM0409 | MSM0410 |
| MSM0411 | MSM0412 | MSM0413 | MSM0414 | MSM0415 |
| MSM0416 | MSM0417 | MSM0418 | MSM0419 | MSM0420 |
| MSM0421 | MSM0422 | MSM0423 | MSM0424 | MSM0425 |
| MSM0426 | MSM0427 | MSM0428 | MSM0429 | MSM0430 |
| MSM0431 | MSM0432 | MSM0433 | MSM0434 | MSM0435 |
| MSM0436 | MSM0437 | MSM0438 | MSM0439 | MSM0440 |
| MSM0441 | MSM0442 | MSM0443 | MSM0444 | MSM0445 |
| MSM0446 | MSM0447 | MSM0448 | MSM0449 | MSM0450 |
| MSM0451 | MSM0452 | MSM0453 | MSM0454 | MSM0455 |
| MSM0456 | MSM0457 | MSM0458 | MSM0459 | MSM0460 |
| MSM0461 | MSM0462 | MSM0463 | MSM0464 | MSM0465 |
| MSM0466 | MSM0467 | MSM0468 | MSM0469 | MSM0470 |
| MSM0471 | MSM0472 | MSM0473 | MSM0474 | MSM0475 |
| MSM0476 | MSM0477 | MSM0478 | MSM0479 | MSM0480 |
| MSM0481 | MSM0482 | MSM0483 | MSM0484 | MSM0485 |
| MSM0486 | MSM0487 | MSM0488 | MSM0489 | MSM0490 |
| MSM0491 | MSM0492 | MSM0493 | MSM0494 | MSM0495 |
| MSM0496 | MSM0497 | MSM0498 | MSM0499 | MSM0500 |
| MSM0501 | MSM0502 | MSM0503 | MSM0504 | MSM0505 |
| MSM0506 | MSM0507 | MSM0508 | MSM0509 | MSM0510 |
| MSM0511 | MSM0512 | MSM0513 | MSM0514 | MSM0515 |
| MSM0516 | MSM0517 | MSM0518 | MSM0519 | MSM0520 |
| MSM0521 | MSM0522 | MSM0523 | MSM0524 | MSM0525 |
| MSM0526 | MSM0527 | MSM0528 | MSM0529 | MSM0530 |
| MSM0531 | MSM0532 | MSM0533 | MSM0534 | MSM0535 |
| MSM0536 | MSM0537 | MSM0538 | MSM0539 | MSM0540 |
| MSM0541 | MSM0542 | MSM0543 | MSM0544 | MSM0545 |
| MSM0546 | MSM0547 | MSM0548 | MSM0549 | MSM0550 |
| MSM0551 | MSM0552 | MSM0553 | MSM0554 | MSM0555 |
| MSM0556 | MSM0557 | MSM0558 | MSM0559 | MSM0560 |
| MSM0561 | MSM0562 | MSM0563 | MSM0564 | MSM0565 |
| MSM0566 | MSM0567 | MSM0568 | MSM0569 | MSM0570 |
| MSM0571 | MSM0572 | MSM0573 | MSM0574 | MSM0575 |
| MSM0576 | MSM0577 | MSM0578 | MSM0579 | MSM0580 |
| MSM0581 | MSM0582 | MSM0583 | MSM0584 | MSM0585 |
| MSM0586 | MSM0587 | MSM0588 | MSM0589 | MSM0590 |
| MSM0591 | MSM0592 | MSM0593 | MSM0594 | MSM0595 |
| MSM0596 | MSM0597 | MSM0598 | MSM0599 | MSM0600 |
| MSM0601 | MSM0602 | MSM0603 | MSM0604 | MSM0605 |
| MSM0606 | MSM0607 | MSM0608 | MSM0609 | MSM0610 |
| MSM0611 | MSM0612 | MSM0613 | MSM0614 | MSM0615 |
| MSM0616 | MSM0617 | MSM0618 | MSM0619 | MSM0620 |
| MSM0621 | MSM0622 | MSM0623 | MSM0624 | MSM0625 |
| MSM0626 | MSM0627 | MSM0628 | MSM0629 | MSM0630 |
| MSM0631 | MSM0632 | MSM0633 | MSM0634 | MSM0635 |
| MSM0636 | MSM0637 | MSM0638 | MSM0639 | MSM0640 |
| MSM0641 | MSM0642 | MSM0643 | MSM0644 | MSM0645 |
| MSM0646 | MSM0647 | MSM0648 | MSM0649 | MSM0650 |
| MSM0651 | MSM0652 | MSM0653 | MSM0654 | MSM0655 |
| MSM0656 | MSM0657 | MSM0658 | MSM0659 | MSM0660 |
| MSM0661 | MSM0662 | MSM0663 | MSM0664 | MSM0665 |
| MSM0666 | MSM0667 | MSM0668 | MSM0669 | MSM0670 |
| MSM0671 | MSM0672 | MSM0673 | MSM0674 | MSM0675 |
| MSM0676 | MSM0677 | MSM0678 | MSM0679 | MSM0680 |
| MSM0681 | MSM0682 | MSM0683 | MSM0684 | MSM0685 |
| MSM0686 | MSM0687 | MSM0688 | MSM0689 | MSM0690 |
| MSM0691 | MSM0692 | MSM0693 | MSM0694 | MSM0695 |
| MSM0696 | MSM0697 | MSM0698 | MSM0699 | MSM0700 |
| MSM0701 | MSM0702 | MSM0703 | MSM0704 | MSM0705 |
| MSM0706 | MSM0707 | MSM0708 | MSM0709 | MSM0710 |
| MSM0711 | MSM0712 | MSM0713 | MSM0714 | MSM0715 |
| MSM0716 | MSM0717 | MSM0718 | MSM0719 | MSM0720 |
| MSM0721 | MSM0722 | MSM0723 | MSM0724 | MSM0725 |
| MSM0726 | MSM0727 | MSM0728 | MSM0729 | MSM0730 |
| MSM0731 | MSM0732 | MSM0733 | MSM0734 | MSM0735 |
| MSM0736 | MSM0737 | MSM0738 | MSM0739 | MSM0740 |
| MSM0741 | MSM0742 | MSM0743 | MSM0744 | MSM0745 |
| MSM0746 | MSM0747 | MSM0748 | MSM0749 | MSM0750 |
| MSM0751 | MSM0752 | MSM0753 | MSM0754 | MSM0755 |
| MSM0756 | MSM0757 | MSM0758 | MSM0759 | MSM0760 |
| MSM0761 | MSM0762 | MSM0763 | MSM0764 | MSM0765 |
| MSM0766 | MSM0767 | MSM0768 | MSM0769 | MSM0770 |
| MSM0771 | MSM0772 | MSM0773 | MSM0774 | MSM0775 |
| MSM0776 | MSM0777 | MSM0778 | MSM0779 | MSM0780 |
| MSM0781 | MSM0782 | MSM0783 | MSM0784 | MSM0785 |
| MSM0786 | MSM0787 | MSM0788 | MSM0789 | MSM0790 |
| MSM0791 | MSM0792 | MSM0793 | MSM0794 | MSM0795 |
| MSM0796 | MSM0797 | MSM0798 | MSM0799 | MSM0800 |
| MSM0801 | MSM0802 | MSM0803 | MSM0804 | MSM0805 |
| MSM0806 | MSM0807 | MSM0808 | MSM0809 | MSM0810 |
| MSM0811 | MSM0812 | MSM0813 | MSM0814 | MSM0815 |
| MSM0816 | MSM0817 | MSM0818 | MSM0819 | MSM0820 |
| MSM0821 | MSM0822 | MSM0823 | MSM0824 | MSM0825 |
| MSM0826 | MSM0827 | MSM0828 | MSM0829 | MSM0830 |
| MSM0831 | MSM0832 | MSM0833 | MSM0834 | MSM0835 |
| MSM0836 | MSM0837 | MSM0838 | MSM0839 | MSM0840 |
| MSM0841 | MSM0842 | MSM0843 | MSM0844 | MSM0845 |
| MSM0846 | MSM0847 | MSM0848 | MSM0849 | MSM0850 |
| MSM0851 | MSM0852 | MSM0853 | MSM0854 | MSM0855 |
| MSM0856 | MSM0857 | MSM0858 | MSM0859 | MSM0860 |
| MSM0861 | MSM0862 | MSM0863 | MSM0864 | MSM0865 |
| MSM0866 | MSM0867 | MSM0868 | MSM0869 | MSM0870 |
| MSM0871 | MSM0872 | MSM0873 | MSM0874 | MSM0875 |
| MSM0876 | MSM0877 | MSM0878 | MSM0879 | MSM0880 |
| MSM0881 | MSM0882 | MSM0883 | MSM0884 | MSM0885 |
| MSM0886 | MSM0887 | MSM0888 | MSM0889 | MSM0890 |
| MSM0891 | MSM0892 | MSM0893 | MSM0894 | MSM0895 |
| MSM0896 | MSM0897 | MSM0898 | MSM0899 | MSM0900 |
| MSM0901 | MSM0902 | MSM0903 | MSM0904 | MSM0905 |
| MSM0906 | MSM0907 | MSM0908 | MSM0909 | MSM0910 |
| MSM0911 | MSM0912 | MSM0913 | MSM0914 | MSM0915 |
| MSM0916 | MSM0917 | MSM0918 | MSM0919 | MSM0920 |
| MSM0921 | MSM0922 | MSM0923 | MSM0924 | MSM0925 |
| MSM0926 | MSM0927 | MSM0928 | MSM0929 | MSM0930 |
| MSM0931 | MSM0932 | MSM0933 | MSM0934 | MSM0935 |
| MSM0936 | MSM0937 | MSM0938 | MSM0939 | MSM0940 |
| MSM0941 | MSM0942 | MSM0943 | MSM0944 | MSM0945 |
| MSM0946 | MSM0947 | MSM0948 | MSM0949 | MSM0950 |
| MSM0951 | MSM0952 | MSM0953 | MSM0954 | MSM0955 |
| MSM0956 | MSM0957 | MSM0958 | MSM0959 | MSM0960 |
| MSM0961 | MSM0962 | MSM0963 | MSM0964 | MSM0965 |
| MSM0966 | MSM0967 | MSM0968 | MSM0969 | MSM0970 |
| MSM0971 | MSM0972 | MSM0973 | MSM0974 | MSM0975 |
| MSM0976 | MSM0977 | MSM0978 | MSM0979 | MSM0980 |
| MSM0981 | MSM0982 | MSM0983 | MSM0984 | MSM0985 |
| MSM0986 | MSM0987 | MSM0988 | MSM0989 | MSM0990 |
| MSM0991 | MSM0992 | MSM0993 | MSM0994 | MSM0995 |
| MSM0996 | MSM0997 | MSM0998 | MSM0999 | MSM1000 |
| MSM1001 | MSM1002 | MSM1003 | MSM1004 | MSM1005 |
| MSM1006 | MSM1007 | MSM1008 | MSM1009 | MSM1010 |
| MSM1011 | MSM1012 | MSM1013 | MSM1014 | MSM1015 |
| MSM1016 | MSM1017 | MSM1018 | MSM1019 | MSM1020 |
| MSM1021 | MSM1022 | MSM1023 | MSM1024 | MSM1025 |
| MSM1026 | MSM1027 | MSM1028 | MSM1029 | MSM1030 |
| MSM1031 | MSM1032 | MSM1033 | MSM1034 | MSM1035 |
| MSM1036 | MSM1037 | MSM1038 | MSM1039 | MSM1040 |
| MSM1041 | MSM1042 | MSM1043 | MSM1044 | MSM1045 |
| MSM1046 | MSM1047 | MSM1048 | MSM1049 | MSM1050 |
| MSM1051 | MSM1052 | MSM1053 | MSM1054 | MSM1055 |
| MSM1056 | MSM1057 | MSM1058 | MSM1059 | MSM1060 |
| MSM1061 | MSM1062 | MSM1063 | MSM1064 | MSM1065 |
| MSM1066 | MSM1067 | MSM1068 | MSM1069 | MSM1070 |
| MSM1071 | MSM1072 | MSM1073 | MSM1074 | MSM1075 |
| MSM1076 | MSM1077 | MSM1078 | MSM1079 | MSM1080 |
| MSM1081 | MSM1082 | MSM1083 | MSM1084 | MSM1085 |
| MSM1086 | MSM1087 | MSM1088 | MSM1089 | MSM1090 |
| MSM1091 | MSM1092 | MSM1093 | MSM1094 | MSM1095 |
| MSM1096 | MSM1097 | MSM1098 | MSM1099 | MSM1100 |
| MSM1101 | MSM1102 | MSM1103 | MSM1104 | MSM1105 |
| MSM1106 | MSM1107 | MSM1108 | MSM1109 | MSM1110 |
| MSM1111 | MSM1112 | MSM1113 | MSM1114 | MSM1115 |
| MSM1116 | MSM1117 | MSM1118 | MSM1119 | MSM1120 |
| MSM1121 | MSM1122 | MSM1123 | MSM1124 | MSM1125 |
| MSM1126 | MSM1127 | MSM1128 | MSM1129 | MSM1130 |
| MSM1131 | MSM1132 | MSM1133 | MSM1134 | MSM1135 |
| MSM1136 | MSM1137 | MSM1138 | MSM1139 | MSM1140 |
| MSM1141 | MSM1142 | MSM1143 | MSM1144 | MSM1145 |
| MSM1146 | MSM1147 | MSM1148 | MSM1149 | MSM1150 |
| MSM1151 | MSM1152 | MSM1153 | MSM1154 | MSM1155 |
| MSM1156 | MSM1157 | MSM1158 | MSM1159 | MSM1160 |
| MSM1161 | MSM1162 | MSM1163 | MSM1164 | MSM1165 |
| MSM1166 | MSM1167 | MSM1168 | MSM1169 | MSM1170 |
| MSM1171 | MSM1172 | MSM1173 | MSM1174 | MSM1175 |
| MSM1176 | MSM1177 | MSM1178 | MSM1179 | MSM1180 |
| MSM1181 | MSM1182 | MSM1183 | MSM1184 | MSM1185 |
| MSM1186 | MSM1187 | MSM1188 | MSM1189 | MSM1190 |
| MSM1191 | MSM1192 | MSM1193 | MSM1194 | MSM1195 |
| MSM1196 | MSM1197 | MSM1198 | MSM1199 | MSM1200 |
| MSM1201 | MSM1202 | MSM1203 | MSM1204 | MSM1205 |
| MSM1206 | MSM1207 | MSM1208 | MSM1209 | MSM1210 |
| MSM1211 | MSM1212 | MSM1213 | MSM1214 | MSM1215 |
| MSM1216 | MSM1217 | MSM1218 | MSM1219 | MSM1220 |
| MSM1221 | MSM1222 | MSM1223 | MSM1224 | MSM1225 |
| MSM1226 | MSM1227 | MSM1228 | MSM1229 | MSM1230 |
| MSM1231 | MSM1232 | MSM1233 | MSM1234 | MSM1235 |
| MSM1236 | MSM1237 | MSM1238 | MSM1239 | MSM1240 |
| MSM1241 | MSM1242 | MSM1243 | MSM1244 | MSM1245 |
| MSM1246 | MSM1247 | MSM1248 | MSM1249 | MSM1250 |
| MSM1251 | MSM1252 | MSM1253 | MSM1254 | MSM1255 |
| MSM1256 | MSM1257 | MSM1258 | MSM1259 | MSM1260 |
| MSM1261 | MSM1262 | MSM1263 | MSM1264 | MSM1265 |
| MSM1266 | MSM1267 | MSM1268 | MSM1269 | MSM1270 |
| MSM1271 | MSM1272 | MSM1273 | MSM1274 | MSM1275 |
| MSM1276 | MSM1277 | MSM1278 | MSM1279 | MSM1280 |
| MSM1281 | MSM1282 | MSM1283 | MSM1284 | MSM1285 |
| MSM1286 | MSM1287 | MSM1288 | MSM1289 | MSM1290 |
| MSM1291 | MSM1292 | MSM1293 | MSM1294 | MSM1295 |
| MSM1296 | MSM1297 | MSM1298 | MSM1299 | MSM1300 |
| MSM1301 | MSM1302 | MSM1303 | MSM1304 | MSM1305 |
| MSM1306 | MSM1307 | MSM1308 | MSM1309 | MSM1310 |
| MSM1311 | MSM1312 | MSM1313 | MSM1314 | MSM1315 |
| MSM1316 | MSM1317 | MSM1318 | MSM1319 | MSM1320 |
| MSM1321 | MSM1322 | MSM1323 | MSM1324 | MSM1325 |
| MSM1326 | MSM1327 | MSM1328 | MSM1329 | MSM1330 |
| MSM1331 | MSM1332 | MSM1333 | MSM1334 | MSM1335 |
| MSM1336 | MSM1337 | MSM1338 | MSM1339 | MSM1340 |
| MSM1341 | MSM1342 | MSM1343 | MSM1344 | MSM1345 |
| MSM1346 | MSM1347 | MSM1348 | MSM1349 | MSM1350 |
| MSM1351 | MSM1352 | MSM1353 | MSM1354 | MSM1355 |
| MSM1356 | MSM1357 | MSM1358 | MSM1359 | MSM1360 |
| MSM1361 | MSM1362 | MSM1363 | MSM1364 | MSM1365 |
| MSM1366 | MSM1367 | MSM1368 | MSM1369 | MSM1370 |
| MSM1371 | MSM1372 | MSM1373 | MSM1374 | MSM1375 |
| MSM1376 | MSM1377 | MSM1378 | MSM1379 | MSM1380 |
| MSM1381 | MSM1382 | MSM1383 | MSM1384 | MSM1385 |
| MSM1386 | MSM1387 | MSM1388 | MSM1389 | MSM1390 |
| MSM1391 | MSM1392 | MSM1393 | MSM1394 | MSM1395 |
| MSM1396 | MSM1397 | MSM1398 | MSM1399 | MSM1400 |
| MSM1401 | MSM1402 | MSM1403 | MSM1404 | MSM1405 |
| MSM1406 | MSM1407 | MSM1408 | MSM1409 | MSM1410 |
| MSM1411 | MSM1412 | MSM1413 | MSM1414 | MSM1415 |
| MSM1416 | MSM1417 | MSM1418 | MSM1419 | MSM1420 |
| MSM1421 | MSM1422 | MSM1423 | MSM1424 | MSM1425 |
| MSM1426 | MSM1427 | MSM1428 | MSM1429 | MSM1430 |
| MSM1431 | MSM1432 | MSM1433 | MSM1434 | MSM1435 |
| MSM1436 | MSM1437 | MSM1438 | MSM1439 | MSM1440 |
| MSM1441 | MSM1442 | MSM1443 | MSM1444 | MSM1445 |
| MSM1446 | MSM1447 | MSM1448 | MSM1449 | MSM1450 |
| MSM1451 | MSM1452 | MSM1453 | MSM1454 | MSM1455 |
| MSM1456 | MSM1457 | MSM1458 | MSM1459 | MSM1460 |
| MSM1461 | MSM1462 | MSM1463 | MSM1464 | MSM1465 |
| MSM1466 | MSM1467 | MSM1468 | MSM1469 | MSM1470 |
| MSM1471 | MSM1472 | MSM1473 | MSM1474 | MSM1475 |
| MSM1476 | MSM1477 | MSM1478 | MSM1479 | MSM1480 |
| MSM1481 | MSM1482 | MSM1483 | MSM1484 | MSM1485 |
| MSM1486 | MSM1487 | MSM1488 | MSM1489 | MSM1490 |
| MSM1491 | MSM1492 | MSM1493 | MSM1494 | MSM1495 |
| MSM1496 | MSM1497 | MSM1498 | MSM1499 | MSM1500 |
| MSM1501 | MSM1502 | MSM1503 | MSM1504 | MSM1505 |
| MSM1506 | MSM1507 | MSM1508 | MSM1509 | MSM1510 |
| MSM1511 | MSM1512 | MSM1513 | MSM1514 | MSM1515 |
| MSM1516 | MSM1517 | MSM1518 | MSM1519 | MSM1520 |
| MSM1521 | MSM1522 | MSM1523 | MSM1524 | MSM1525 |
| MSM1526 | MSM1527 | MSM1528 | MSM1529 | MSM1530 |
| MSM1531 | MSM1532 | MSM1533 | MSM1534 | MSM1535 |
| MSM1536 | MSM1537 | MSM1538 | MSM1539 | MSM1540 |
| MSM1541 | MSM1542 | MSM1543 | MSM1544 | MSM1545 |
| MSM1546 | MSM1547 | MSM1548 | MSM1549 | MSM1550 |
| MSM1551 | MSM1552 | MSM1553 | MSM1554 | MSM1555 |
| MSM1556 | MSM1557 | MSM1558 | MSM1559 | MSM1560 |
| MSM1561 | MSM1562 | MSM1563 | MSM1564 | MSM1565 |
| MSM1566 | MSM1567 | MSM1568 | MSM1569 | MSM1570 |
| MSM1571 | MSM1572 | MSM1573 | MSM1574 | MSM1575 |
| MSM1576 | MSM1577 | MSM1578 | MSM1579 | MSM1580 |
| MSM1581 | MSM1582 | MSM1583 | MSM1584 | MSM1585 |
| MSM1586 | MSM1587 | MSM1588 | MSM1589 | MSM1590 |
| MSM1591 | MSM1592 | MSM1593 | MSM1594 | MSM1595 |
| MSM1596 | MSM1597 | MSM1598 | MSM1599 | MSM1600 |
| MSM1601 | MSM1602 | MSM1603 | MSM1604 | MSM1605 |
| MSM1606 | MSM1607 | MSM1608 | MSM1609 | MSM1610 |
| MSM1611 | MSM1612 | MSM1613 | MSM1614 | MSM1615 |
| MSM1616 | MSM1617 | MSM1618 | MSM1619 | MSM1620 |
| MSM1621 | MSM1622 | MSM1623 | MSM1624 | MSM1625 |
| MSM1626 | MSM1627 | MSM1628 | MSM1629 | MSM1630 |
| MSM1631 | MSM1632 | MSM1633 | MSM1634 | MSM1635 |
| MSM1636 | MSM1637 | MSM1638 | MSM1639 | MSM1640 |
| MSM1641 | MSM1642 | MSM1643 | MSM1644 | MSM1645 |
| MSM1646 | MSM1647 | MSM1648 | MSM1649 | MSM1650 |
| MSM1651 | MSM1652 | MSM1653 | MSM1654 | MSM1655 |
| MSM1656 | MSM1657 | MSM1658 | MSM1659 | MSM1660 |
| MSM1661 | MSM1662 | MSM1663 | MSM1664 | MSM1665 |
| MSM1666 | MSM1667 | MSM1668 | MSM1669 | MSM1670 |
| MSM1671 | MSM1672 | MSM1673 | MSM1674 | MSM1675 |
| MSM1676 | MSM1677 | MSM1678 | MSM1679 | MSM1680 |
| MSM1681 | MSM1682 | MSM1683 | MSM1684 | MSM1685 |
| MSM1686 | MSM1687 | MSM1688 | MSM1689 | MSM1690 |
| MSM1691 | MSM1692 | MSM1693 | MSM1694 | MSM1695 |
| MSM1696 | MSM1697 | MSM1698 | MSM1699 | MSM1700 |
| MSM1701 | MSM1702 | MSM1703 | MSM1704 | MSM1705 |
| MSM1706 | MSM1707 | MSM1708 | MSM1709 | MSM1710 |
| MSM1711 | MSM1712 | MSM1713 | MSM1714 | MSM1715 |
| MSM1716 | MSM1717 | MSM1718 | MSM1719 | MSM1720 |
| MSM1721 | MSM1722 | MSM1723 | MSM1724 | MSM1725 |
| MSM1726 | MSM1727 | MSM1728 | MSM1729 | MSM1730 |
| MSM1731 | MSM1732 | MSM1733 | MSM1734 | MSM1735 |
| MSM1736 | MSM1737 | MSM1738 | MSM1739 | MSM1740 |
| MSM1741 | MSM1742 | MSM1743 | MSM1744 | MSM1745 |
| MSM1746 | MSM1747 | MSM1748 | MSM1749 | MSM1750 |
| MSM1751 | MSM1752 | MSM1753 | MSM1754 | MSM1755 |
| MSM1756 | MSM1757 | MSM1758 | MSM1759 | MSM1760 |
| MSM1761 | MSM1762 | MSM1763 | MSM1764 | MSM1765 |
| MSM1766 | MSM1767 | MSM1768 | MSM1769 | MSM1770 |
| MSM1771 | MSM1772 | MSM1773 | MSM1774 | MSM1775 |
| MSM1776 | MSM1777 | MSM1778 | MSM1779 | MSM1780 |
| MSM1781 | MSM1782 | MSM1783 | MSM1784 | MSM1785 |
| MSM1786 | MSM1787 | MSM1788 | MSM1789 | MSM1790 |
| MSM1791 | MSM1792 | MSM1793 | MSM1794 | MSM1795 |

TABLE B
SEQ ID NOs for nucleic acid sequences of ALPs and
putative ALPs from M. smithii strain PS

| Locus tag | Annotation | SEQ ID NO | GeneID |
|---|---|---|---|
| MSM0031 | ALP | 1 | 5216283 |
| MSM0051 | ALP | 3 | 5215780 |
| MSM0052 | ALP | 5 | 5215781 |
| MSM0057 | ALP | 7 | 5215760 |
| MSM0092 | putative ALP | 9 | 5216811 |
| MSM0159 | ALP | 11 | 5216808 |
| MSM0173 | ALP | 13 | 5216543 |
| MSM0221 | ALP | 15 | 5216462 |
| MSM0266 | ALP | 17 | 5216710 |
| MSM0281 | putative ALP | 19 | 5216489 |
| MSM0282 | ALP | 21 | 5216741 |
| MSM0337 | putative ALP | 23 | 5216748 |
| MSM0411 | ALP | 25 | 5216551 |
| MSM0412 | ALP | 27 | 5216552 |
| MSM0461 | ALP | 29 | 5216168 |
| MSM0580 | ALP | 31 | 5217434 |
| MSM0616 | ALP | 33 | 5217327 |
| MSM0884 | ALP | 35 | 5215891 |
| MSM0885 | ALP | 37 | 5216018 |
| MSM0957 | ALP | 39 | 5216076 |
| MSM0995 | ALP | 41 | 5217000 |
| MSM0996 | ALP | 43 | 5217001 |
| MSM1111 | ALP | 45 | 5216938 |
| MSM1112 | ALP | 47 | 5216939 |
| MSM1113 | ALP | 49 | 5216940 |
| MSM1114 | ALP | 51 | 5216941 |
| MSM1116 | ALP | 53 | 5216944 |
| MSM1168 | putative ALP | 55 | 5217402 |
| MSM1188 | ALP | 57 | 5216254 |
| MSM1282 | putative ALP | 59 | 5217426 |
| MSM1305 | ALP | 61 | 5215879 |
| MSM1306 | ALP | 63 | 5215880 |
| MSM1397 | ALP | 65 | 5216612 |
| MSM1398 | ALP | 67 | 5216613 |
| MSM1399 | ALP | 69 | 5216614 |
| MSM1485 | putative ALP | 71 | 5216177 |
| MSM1533 | ALP | 73 | 5216447 |
| MSM1534 | ALP | 75 | 5216448 |
| MSM1554 | putative ALP | 77 | 5216474 |
| MSM1567 | ALP | 79 | 5216067 |
| MSM1585 | ALP | 81 | 5217144 |
| MSM1586 | ALP | 83 | 5217145 |
| MSM1587 | ALP | 85 | 5217146 |
| MSM1590 | ALP | 87 | 5217149 |
| MSM1709 | ALP | 89 | 5217342 |
| MSM1716 | ALP | 91 | 5217453 |
| MSM1735 | ALP | 93 | 5215918 |
| MSM1738 | putative ALP | 95 | 5215921 |

TABLE C
SEQ ID NOs for amino sequences of ALPs and
putative ALPs from M. smithii strain PS

| Locus tag | Annotation | SEQ ID NO | Protein ID |
|---|---|---|---|
| MSM0031 | ALP | 2 | YP_001272604.1 |
| MSM0051 | ALP | 4 | YP_001272624.1 |
| MSM0052 | ALP | 6 | YP_001272625.1 |
| MSM0057 | ALP | 8 | YP_001272665.1 |
| MSM0092 | putative ALP | 10 | YP_0012726321.1 |
| MSM0159 | ALP | 12 | YP_001272732.1 |
| MSM0173 | ALP | 14 | YP_001272746.1 |
| MSM0221 | ALP | 16 | YP_001272794.1 |
| MSM0266 | ALP | 18 | YP_001272839.1 |
| MSM0281 | putative ALP | 20 | YP_001272854.1 |
| MSM0282 | ALP | 22 | YP_001272855.1 |
| MSM0337 | putative ALP | 24 | YP_001272910.1 |
| MSM0411 | ALP | 26 | YP_001272984.1 |
| MSM0412 | ALP | 28 | YP_001272985.1 |
| MSM0461 | ALP | 30 | YP_001273034.1 |
| MSM0580 | ALP | 32 | YP_001273153.1 |
| MSM0616 | ALP | 34 | YP_001273189.1 |
| MSM0884 | ALP | 36 | YP_001273457.1 |
| MSM0885 | ALP | 38 | YP_001273458.1 |
| MSM0957 | ALP | 40 | YP_001273530.1 |
| MSM0995 | ALP | 42 | YP_001273568.1 |
| MSM0996 | ALP | 44 | YP_001273569.1 |
| MSM1111 | ALP | 46 | YP_001273684.1 |
| MSM1112 | ALP | 48 | YP_001273685.1 |
| MSM1113 | ALP | 50 | YP_001273686.1 |
| MSM1114 | ALP | 52 | YP_001273687.1 |
| MSM1116 | ALP | 54 | YP_001273689.1 |
| MSM1168 | putative ALP | 56 | YP_001273741.1 |
| MSM1188 | ALP | 58 | YP_001273761.1 |
| MSM1282 | putative ALP | 60 | YP_001273855.1 |
| MSM1305 | ALP | 62 | YP_001273878.1 |
| MSM1306 | ALP | 64 | YP_001273879.1 |
| MSM1397 | ALP | 66 | YP_001273970.1 |
| MSM1398 | ALP | 68 | YP_001273971.1 |
| MSM1399 | ALP | 70 | YP_001273972.1 |
| MSM1485 | putative ALP | 72 | YP_001274058.1 |
| MSM1533 | ALP | 74 | YP_001274106.1 |
| MSM1534 | ALP | 76 | YP_001274107.1 |
| MSM1554 | putative ALP | 78 | YP_001274127.1 |
| MSM1567 | ALP | 80 | YP_001274140.1 |
| MSM1585 | ALP | 82 | YP_001274158.1 |
| MSM1586 | ALP | 84 | YP_001274159.1 |
| MSM1587 | ALP | 86 | YP_001274160.1 |
| MSM1590 | ALP | 88 | YP_001274163.1 |
| MSM1709 | ALP | 90 | YP_001274282.1 |
| MSM1716 | ALP | 92 | YP_001274289.1 |
| MSM1735 | ALP | 94 | YP_001274308.1 |
| MSM1738 | putative ALP | 96 | YP_001274311.1 |

TABLE D
SEQ ID NOs for nucleic acid sequences of ALPs and
putative ALPs from other M. smithii strains

| | | SEQ |
|---|---|---|
| | | ID |
| Strain | ALP Gene Number | NO |
| METSMIALI | METSMIALI_0078 | 97 |
| METSMIALI | METSMIALI_0079 | 98 |
| METSMIALI | METSMIALI_0100 | 99 |
| METSMIALI | METSMIALI_0150 | 100 |
| METSMIALI | METSMIALI_0152 | 101 |
| METSMIALI | METSMIALI_0198 | 102 |
| METSMIALI | METSMIALI_0269 | 103 |
| METSMIALI | METSMIALI_0270 | 104 |
| METSMIALI | METSMIALI_0307 | 105 |
| METSMIALI | METSMIALI_0308 | 106 |
| METSMIALI | METSMIALI_0328 | 107 |
| METSMIALI | METSMIALI_0370 | 108 |
| METSMIALI | METSMIALI_0373 | 109 |
| METSMIALI | METSMIALI_0480 | 110 |
| METSMIALI | METSMIALI_0510 | 111 |
| METSMIALI | METSMIALI_0551 | 112 |
| METSMIALI | METSMIALI_0670 | 113 |
| METSMIALI | METSMIALI_0776 | 114 |
| METSMIALI | METSMIALI_0810 | 115 |
| METSMIALI | METSMIALI_0845 | 116 |
| METSMIALI | METSMIALI_0884 | 117 |
| METSMIALI | METSMIALI_0998 | 118 |
| METSMIALI | METSMIALI_0999 | 119 |
| METSMIALI | METSMIALI_1053 | 120 |
| METSMIALI | METSMIALI_1073 | 121 |
| METSMIALI | METSMIALI_1074 | 122 |
| METSMIALI | METSMIALI_1175 | 123 |
| METSMIALI | METSMIALI_1199 | 124 |
| METSMIALI | METSMIALI_1452 | 125 |
| METSMIALI | METSMIALI_1616 | 126 |
| METSMIALI | METSMIALI_1617 | 127 |
| METSMIF1 | METSMIF1_0060 | 128 |
| METSMIF1 | METSMIF1_0061 | 129 |
| METSMIF1 | METSMIF1_0226 | 130 |
| METSMIF1 | METSMIF1_0475 | 131 |
| METSMIF1 | METSMIF1_0593 | 132 |
| METSMIF1 | METSMIF1_0614 | 133 |
| METSMIF1 | METSMIF1_0615 | 134 |
| METSMIF1 | METSMIF1_0669 | 135 |
| METSMIF1 | METSMIF1_0670 | 136 |
| METSMIF1 | METSMIF1_0671 | 137 |
| METSMIF1 | METSMIF1_0672 | 138 |
| METSMIF1 | METSMIF1_0673 | 139 |
| METSMIF1 | METSMIF1_0788 | 140 |
| METSMIF1 | METSMIF1_0827 | 141 |
| METSMIF1 | METSMIF1_0861 | 142 |
| METSMIF1 | METSMIF1_0893 | 143 |
| METSMIF1 | METSMIF1_0894 | 144 |
| METSMIF1 | METSMIF1_0991 | 145 |
| METSMIF1 | METSMIF1_1105 | 146 |
| METSMIF1 | METSMIF1_1176 | 147 |
| METSMIF1 | METSMIF1_1264 | 148 |
| METSMIF1 | METSMIF1_1284 | 149 |
| METSMIF1 | METSMIF1_1287 | 150 |
| METSMIF1 | METSMIF1_1359 | 151 |
| METSMIF1 | METSMIF1_1379 | 152 |
| METSMIF1 | METSMIF1_1380 | 153 |
| METSMIF1 | METSMIF1_1381 | 154 |
| METSMIF1 | METSMIF1_1489 | 155 |
| METSMIF1 | METSMIF1_1536 | 156 |
| METSMIF1 | METSMIF1_1538 | 157 |
| METSMIF1 | METSMIF1_1583 | 158 |
| METSMIF1 | METSMIF1_1598 | 159 |
| METSMIF1 | METSMIF1_1599 | 160 |
| METSMIF1 | METSMIF1_1672 | 161 |
| METSMITS145A | METSMITS145A_0074 | 162 |
| METSMITS145A | METSMITS145A_0093 | 163 |
| METSMITS145A | METSMITS145A_0096 | 164 |
| METSMITS145A | METSMITS145A_0133 | 165 |
| METSMITS145A | METSMITS145A_0153 | 166 |
| METSMITS145A | METSMITS145A_0154 | 167 |
| METSMITS145A | METSMITS145A_0155 | 168 |
| METSMITS145A | METSMITS145A_0199 | 169 |
| METSMITS145A | METSMITS145A_0277 | 170 |
| METSMITS145A | METSMITS145A_0323 | 171 |
| METSMITS145A | METSMITS145A_0326 | 172 |
| METSMITS145A | METSMITS145A_0336 | 173 |
| METSMITS145A | METSMITS145A_0374 | 174 |
| METSMITS145A | METSMITS145A_0393 | 175 |
| METSMITS145A | METSMITS145A_0394 | 176 |
| METSMITS145A | METSMITS145A_0395 | 177 |
| METSMITS145A | METSMITS145A_0542 | 178 |
| METSMITS145A | METSMITS145A_0543 | 179 |
| METSMITS145A | METSMITS145A_0545 | 180 |
| METSMITS145A | METSMITS145A_0704 | 181 |
| METSMITS145A | METSMITS145A_0874 | 182 |
| METSMITS145A | METSMITS145A_0875 | 183 |
| METSMITS145A | METSMITS145A_0876 | 184 |
| METSMITS145A | METSMITS145A_0967 | 185 |
| METSMITS145A | METSMITS145A_0968 | 186 |
| METSMITS145A | METSMITS145A_0997 | 187 |
| METSMITS145A | METSMITS145A_0998 | 188 |
| METSMITS145A | METSMITS145A_1005 | 189 |
| METSMITS145A | METSMITS145A_1043 | 190 |
| METSMITS145A | METSMITS145A_1083 | 191 |
| METSMITS145A | METSMITS145A_1196 | 192 |
| METSMITS145A | METSMITS145A_1253 | 193 |
| METSMITS145A | METSMITS145A_1254 | 194 |
| METSMITS145A | METSMITS145A_1277 | 195 |
| METSMITS145A | METSMITS145A_1377 | 196 |
| METSMITS145A | METSMITS145A_1399 | 197 |
| METSMITS145A | METSMITS145A_1400 | 198 |
| METSMITS145A | METSMITS145A_1401 | 199 |
| METSMITS145A | METSMITS145A_1497 | 200 |
| METSMITS145A | METSMITS145A_1498 | 201 |
| METSMITS145A | METSMITS145A_1621 | 202 |
| METSMITS145A | METSMITS145A_1661 | 203 |
| METSMITS145A | METSMITS145A_1684 | 204 |
| METSMITS145A | METSMITS145A_1696 | 205 |
| METSMITS145A | METSMITS145A_1697 | 206 |
| METSMITS145A | METSMITS145A_1716 | 207 |
| METSMITS145A | METSMITS145A_1718 | 208 |
| METSMITS145B | METSMITS145B_0009 | 209 |
| METSMITS145B | METSMITS145B_0030 | 210 |
| METSMITS145B | METSMITS145B_0031 | 211 |
| METSMITS145B | METSMITS145B_0032 | 212 |
| METSMITS145B | METSMITS145B_0033 | 213 |
| METSMITS145B | METSMITS145B_0034 | 214 |
| METSMITS145B | METSMITS145B_0079 | 215 |
| METSMITS145B | METSMITS145B_0154 | 216 |
| METSMITS145B | METSMITS145B_0202 | 217 |
| METSMITS145B | METSMITS145B_0205 | 218 |
| METSMITS145B | METSMITS145B_0256 | 219 |
| METSMITS145B | METSMITS145B_0257 | 220 |
| METSMITS145B | METSMITS145B_0274 | 221 |
| METSMITS145B | METSMITS145B_0275 | 222 |
| METSMITS145B | METSMITS145B_0428 | 223 |
| METSMITS145B | METSMITS145B_0429 | 224 |
| METSMITS145B | METSMITS145B_0431 | 225 |
| METSMITS145B | METSMITS145B_0599 | 226 |
| METSMITS145B | METSMITS145B_0782 | 227 |
| METSMITS145B | METSMITS145B_0783 | 228 |
| METSMITS145B | METSMITS145B_0784 | 229 |
| METSMITS145B | METSMITS145B_0793 | 230 |
| METSMITS145B | METSMITS145B_0800 | 231 |
| METSMITS145B | METSMITS145B_0928 | 232 |
| METSMITS145B | METSMITS145B_0929 | 233 |
| METSMITS145B | METSMITS145B_1027 | 234 |
| METSMITS145B | METSMITS145B_1028 | 235 |
| METSMITS145B | METSMITS145B_1092 | 236 |
| METSMITS145B | METSMITS145B_1093 | 237 |
| METSMITS145B | METSMITS145B_1094 | 238 |
| METSMITS145B | METSMITS145B_1095 | 239 |
| METSMITS145B | METSMITS145B_1220 | 240 |
| METSMITS145B | METSMITS145B_1227 | 241 |
| METSMITS145B | METSMITS145B_1270 | 242 |
| METSMITS145B | METSMITS145B_1314 | 243 |
| METSMITS145B | METSMITS145B_1435 | 244 |
| METSMITS145B | METSMITS145B_1495 | 245 |
| METSMITS145B | METSMITS145B_1522 | 246 |
| METSMITS145B | METSMITS145B_1631 | 247 |
| METSMITS145B | METSMITS145B_1653 | 248 |
| METSMITS145B | METSMITS145B_1665 | 249 |
| METSMITS145B | METSMITS145B_1666 | 250 |
| METSMITS145B | METSMITS145B_1681 | 251 |
| METSMITS145B | METSMITS145B_1703 | 252 |
| METSMITS145B | METSMITS145B_1705 | 253 |
| METSMITS145B | METSMITS145B_1832 | 254 |
| METSMITS145B | METSMITS145B_1852 | 255 |
| METSMITS145B | METSMITS145B_1854 | 256 |
| METSMITS146A | METSMITS146A_0023 | 257 |
| METSMITS146A | METSMITS146A_0024 | 258 |
| METSMITS146A | METSMITS146A_0025 | 259 |
| METSMITS146A | METSMITS146A_0026 | 260 |
| METSMITS146A | METSMITS146A_0069 | 261 |
| METSMITS146A | METSMITS146A_0146 | 262 |
| METSMITS146A | METSMITS146A_0193 | 263 |
| METSMITS146A | METSMITS146A_0194 | 264 |
| METSMITS146A | METSMITS146A_0196 | 265 |
| METSMITS146A | METSMITS146A_0244 | 266 |
| METSMITS146A | METSMITS146A_0245 | 267 |
| METSMITS146A | METSMITS146A_0263 | 268 |
| METSMITS146A | METSMITS146A_0335 | 269 |
| METSMITS146A | METSMITS146A_0336 | 270 |
| METSMITS146A | METSMITS146A_0338 | 271 |
| METSMITS146A | METSMITS146A_0588 | 272 |
| METSMITS146A | METSMITS146A_0756 | 273 |
| METSMITS146A | METSMITS146A_0757 | 274 |
| METSMITS146A | METSMITS146A_0758 | 275 |
| METSMITS146A | METSMITS146A_0803 | 276 |
| METSMITS146A | METSMITS146A_0928 | 277 |
| METSMITS146A | METSMITS146A_1029 | 278 |
| METSMITS146A | METSMITS146A_1030 | 279 |
| METSMITS146A | METSMITS146A_1060 | 280 |
| METSMITS146A | METSMITS146A_1061 | 281 |
| METSMITS146A | METSMITS146A_1068 | 282 |
| METSMITS146A | METSMITS146A_1105 | 283 |
| METSMITS146A | METSMITS146A_1146 | 284 |
| METSMITS146A | METSMITS146A_1147 | 285 |
| METSMITS146A | METSMITS146A_1265 | 286 |
| METSMITS146A | METSMITS146A_1321 | 287 |
| METSMITS146A | METSMITS146A_1346 | 288 |
| METSMITS146A | METSMITS146A_1471 | 289 |
| METSMITS146A | METSMITS146A_1562 | 290 |
| METSMITS146A | METSMITS146A_1563 | 291 |
| METSMITS146A | METSMITS146A_1586 | 292 |
| METSMITS146A | METSMITS146A_1600 | 293 |
| METSMITS146A | METSMITS146A_1619 | 294 |
| METSMITS146A | METSMITS146A_1620 | 295 |
| METSMITS146A | METSMITS146A_1622 | 296 |
| METSMITS146A | METSMITS146A_1623 | 297 |
| METSMITS146A | METSMITS146A_1767 | 298 |
| METSMITS146A | METSMITS146A_1769 | 299 |
| METSMITS146A | METSMITS146A_1810 | 300 |
| METSMITS146B | METSMITS146B_0012 | 301 |
| METSMITS146B | METSMITS146B_0013 | 302 |
| METSMITS146B | METSMITS146B_0034 | 303 |
| METSMITS146B | METSMITS146B_0035 | 304 |
| METSMITS146B | METSMITS146B_0036 | 305 |
| METSMITS146B | METSMITS146B_0077 | 306 |
| METSMITS146B | METSMITS146B_0158 | 307 |
| METSMITS146B | METSMITS146B_0203 | 308 |
| METSMITS146B | METSMITS146B_0205 | 309 |
| METSMITS146B | METSMITS146B_0254 | 310 |
| METSMITS146B | METSMITS146B_0271 | 311 |
| METSMITS146B | METSMITS146B_0422 | 312 |
| METSMITS146B | METSMITS146B_0424 | 313 |
| METSMITS146B | METSMITS146B_0584 | 314 |
| METSMITS146B | METSMITS146B_0759 | 315 |
| METSMITS146B | METSMITS146B_0760 | 316 |
| METSMITS146B | METSMITS146B_0761 | 317 |
| METSMITS146B | METSMITS146B_0791 | 318 |
| METSMITS146B | METSMITS146B_0919 | 319 |
| METSMITS146B | METSMITS146B_0920 | 320 |
| METSMITS146B | METSMITS146B_1017 | 321 |
| METSMITS146B | METSMITS146B_1018 | 322 |
| METSMITS146B | METSMITS146B_1049 | 323 |
| METSMITS146B | METSMITS146B_1056 | 324 |
| METSMITS146B | METSMITS146B_1096 | 325 |
| METSMITS146B | METSMITS146B_1137 | 326 |
| METSMITS146B | METSMITS146B_1253 | 327 |
| METSMITS146B | METSMITS146B_1254 | 328 |
| METSMITS146B | METSMITS146B_1313 | 329 |
| METSMITS146B | METSMITS146B_1335 | 330 |
| METSMITS146B | METSMITS146B_1431 | 331 |
| METSMITS146B | METSMITS146B_1455 | 332 |
| METSMITS146B | METSMITS146B_1574 | 333 |
| METSMITS146B | METSMITS146B_1575 | 334 |
| METSMITS146B | METSMITS146B_1601 | 335 |
| METSMITS146B | METSMITS146B_1614 | 336 |
| METSMITS146B | METSMITS146B_1633 | 337 |
| METSMITS146B | METSMITS146B_1635 | 338 |
| METSMITS146B | METSMITS146B_1636 | 339 |
| METSMITS146B | METSMITS146B_1766 | 340 |
| METSMITS146B | METSMITS146B_1785 | 341 |
| METSMITS146C | METSMITS146C_0021 | 342 |
| METSMITS146C | METSMITS146C_0043 | 343 |
| METSMITS146C | METSMITS146C_0044 | 344 |
| METSMITS146C | METSMITS146C_0046 | 345 |
| METSMITS146C | METSMITS146C_0047 | 346 |
| METSMITS146C | METSMITS146C_0048 | 347 |
| METSMITS146C | METSMITS146C_0049 | 348 |
| METSMITS146C | METSMITS146C_0050 | 349 |
| METSMITS146C | METSMITS146C_0051 | 350 |
| METSMITS146C | METSMITS146C_0052 | 351 |
| METSMITS146C | METSMITS146C_0101 | 352 |
| METSMITS146C | METSMITS146C_0168 | 353 |
| METSMITS146C | METSMITS146C_0273 | 354 |
| METSMITS146C | METSMITS146C_0274 | 355 |
| METSMITS146C | METSMITS146C_0329 | 356 |
| METSMITS146C | METSMITS146C_0330 | 357 |
| METSMITS146C | METSMITS146C_0331 | 358 |
| METSMITS146C | METSMITS146C_0333 | 359 |
| METSMITS146C | METSMITS146C_0355 | 360 |
| METSMITS146C | METSMITS146C_0356 | 361 |
| METSMITS146C | METSMITS146C_0357 | 362 |
| METSMITS146C | METSMITS146C_0358 | 363 |
| METSMITS146C | METSMITS146C_0359 | 364 |
| METSMITS146C | METSMITS146C_0360 | 365 |
| METSMITS146C | METSMITS146C_0361 | 366 |
| METSMITS146C | METSMITS146C_0387 | 367 |
| METSMITS146C | METSMITS146C_0388 | 368 |
| METSMITS146C | METSMITS146C_0531 | 369 |
| METSMITS146C | METSMITS146C_0532 | 370 |
| METSMITS146C | METSMITS146C_0533 | 371 |
| METSMITS146C | METSMITS146C_0534 | 372 |
| METSMITS146C | METSMITS146C_0793 | 373 |
| METSMITS146C | METSMITS146C_0960 | 374 |
| METSMITS146C | METSMITS146C_1020 | 375 |
| METSMITS146C | METSMITS146C_1043 | 376 |
| METSMITS146C | METSMITS146C_1044 | 377 |
| METSMITS146C | METSMITS146C_1045 | 378 |
| METSMITS146C | METSMITS146C_1046 | 379 |
| METSMITS146C | METSMITS146C_1047 | 380 |
| METSMITS146C | METSMITS146C_1054 | 381 |
| METSMITS146C | METSMITS146C_1102 | 382 |
| METSMITS146C | METSMITS146C_1103 | 383 |
| METSMITS146C | METSMITS146C_1149 | 384 |
| METSMITS146C | METSMITS146C_1310 | 385 |
| METSMITS146C | METSMITS146C_1311 | 386 |
| METSMITS146C | METSMITS146C_1312 | 387 |
| METSMITS146C | METSMITS146C_1313 | 388 |
| METSMITS146C | METSMITS146C_1314 | 389 |
| METSMITS146C | METSMITS146C_1374 | 390 |
| METSMITS146C | METSMITS146C_1400 | 391 |
| METSMITS146C | METSMITS146C_1514 | 392 |
| METSMITS146C | METSMITS146C_1538 | 393 |
| METSMITS146C | METSMITS146C_1539 | 394 |
| METSMITS146C | METSMITS146C_1540 | 395 |
| METSMITS146C | METSMITS146C_1541 | 396 |
| METSMITS146C | METSMITS146C_1542 | 397 |
| METSMITS146C | METSMITS146C_1557 | 398 |
| METSMITS146C | METSMITS146C_1558 | 399 |
| METSMITS146C | METSMITS146C_1559 | 400 |
| METSMITS146C | METSMITS146C_1663 | 401 |
| METSMITS146C | METSMITS146C_1664 | 402 |
| METSMITS146C | METSMITS146C_1665 | 403 |
| METSMITS146C | METSMITS146C_1667 | 404 |
| METSMITS146C | METSMITS146C_1870 | 405 |
| METSMITS146C | METSMITS146C_1920 | 406 |
| METSMITS146C | METSMITS146C_1921 | 407 |
| METSMITS146C | METSMITS146C_1922 | 408 |
| METSMITS146C | METSMITS146C_1923 | 409 |
| METSMITS146C | METSMITS146C_1924 | 410 |
| METSMITS146C | METSMITS146C_1950 | 411 |
| METSMITS146C | METSMITS146C_1970 | 412 |
| METSMITS146C | METSMITS146C_1996 | 413 |
| METSMITS146C | METSMITS146C_1997 | 414 |
| METSMITS146C | METSMITS146C_1998 | 415 |
| METSMITS146C | METSMITS146C_2004 | 416 |
| METSMITS146C | METSMITS146C_2005 | 417 |
| METSMITS146C | METSMITS146C_2006 | 418 |
| METSMITS146C | METSMITS146C_2007 | 419 |
| METSMITS146C | METSMITS146C_2008 | 420 |
| METSMITS146C | METSMITS146C_2009 | 421 |
| METSMITS146C | METSMITS146C_2010 | 422 |
| METSMITS146C | METSMITS146C_2011 | 423 |
| METSMITS146C | METSMITS146C_2152 | 424 |
| METSMITS146C | METSMITS146C_2174 | 425 |
| METSMITS146C | METSMITS146C_2175 | 426 |
| METSMITS146C | METSMITS146C_2176 | 427 |
| METSMITS146C | METSMITS146C_2177 | 428 |
| METSMITS146C | METSMITS146C_2180 | 429 |
| METSMITS146C | METSMITS146C_2274 | 430 |
| METSMITS146D | METSMITS146D_0020 | 431 |
| METSMITS146D | METSMITS146D_0021 | 432 |
| METSMITS146D | METSMITS146D_0022 | 433 |
| METSMITS146D | METSMITS146D_0060 | 434 |
| METSMITS146D | METSMITS146D_0139 | 435 |
| METSMITS146D | METSMITS146D_0140 | 436 |
| METSMITS146D | METSMITS146D_0187 | 437 |
| METSMITS146D | METSMITS146D_0189 | 438 |
| METSMITS146D | METSMITS146D_0200 | 439 |
| METSMITS146D | METSMITS146D_0237 | 440 |
| METSMITS146D | METSMITS146D_0255 | 441 |
| METSMITS146D | METSMITS146D_0318 | 442 |
| METSMITS146D | METSMITS146D_0320 | 443 |
| METSMITS146D | METSMITS146D_0483 | 444 |
| METSMITS146D | METSMITS146D_0657 | 445 |
| METSMITS146D | METSMITS146D_0658 | 446 |
| METSMITS146D | METSMITS146D_0659 | 447 |
| METSMITS146D | METSMITS146D_0687 | 448 |
| METSMITS146D | METSMITS146D_0810 | 449 |
| METSMITS146D | METSMITS146D_0907 | 450 |
| METSMITS146D | METSMITS146D_0908 | 451 |
| METSMITS146D | METSMITS146D_0937 | 452 |
| METSMITS146D | METSMITS146D_0938 | 453 |
| METSMITS146D | METSMITS146D_0945 | 454 |
| METSMITS146D | METSMITS146D_0981 | 455 |
| METSMITS146D | METSMITS146D_1020 | 456 |
| METSMITS146D | METSMITS146D_1137 | 457 |
| METSMITS146D | METSMITS146D_1138 | 458 |
| METSMITS146D | METSMITS146D_1139 | 459 |
| METSMITS146D | METSMITS146D_1196 | 460 |
| METSMITS146D | METSMITS146D_1220 | 461 |
| METSMITS146D | METSMITS146D_1319 | 462 |
| METSMITS146D | METSMITS146D_1341 | 463 |
| METSMITS146D | METSMITS146D_1441 | 464 |
| METSMITS146D | METSMITS146D_1465 | 465 |
| METSMITS146D | METSMITS146D_1477 | 466 |
| METSMITS146D | METSMITS146D_1497 | 467 |
| METSMITS146D | METSMITS146D_1499 | 468 |
| METSMITS146D | METSMITS146D_1628 | 469 |
| METSMITS146D | METSMITS146D_1629 | 470 |
| METSMITS146D | METSMITS146D_1648 | 471 |
| METSMITS146D | METSMITS146D_1651 | 472 |
| METSMITS146D | METSMITS146D_1691 | 473 |
| METSMITS146E | METSMITS146E_0040 | 474 |
| METSMITS146E | METSMITS146E_0041 | 475 |
| METSMITS146E | METSMITS146E_0047 | 476 |
| METSMITS146E | METSMITS146E_0085 | 477 |
| METSMITS146E | METSMITS146E_0164 | 478 |
| METSMITS146E | METSMITS146E_0211 | 479 |
| METSMITS146E | METSMITS146E_0213 | 480 |
| METSMITS146E | METSMITS146E_0224 | 481 |
| METSMITS146E | METSMITS146E_0273 | 482 |
| METSMITS146E | METSMITS146E_0289 | 483 |
| METSMITS146E | METSMITS146E_0374 | 484 |
| METSMITS146E | METSMITS146E_0421 | 485 |
| METSMITS146E | METSMITS146E_0422 | 486 |
| METSMITS146E | METSMITS146E_0602 | 487 |
| METSMITS146E | METSMITS146E_0603 | 488 |
| METSMITS146E | METSMITS146E_0604 | 489 |
| METSMITS146E | METSMITS146E_0788 | 490 |
| METSMITS146E | METSMITS146E_0789 | 491 |
| METSMITS146E | METSMITS146E_0791 | 492 |
| METSMITS146E | METSMITS146E_0856 | 493 |
| METSMITS146E | METSMITS146E_0857 | 494 |
| METSMITS146E | METSMITS146E_0974 | 495 |
| METSMITS146E | METSMITS146E_1009 | 496 |
| METSMITS146E | METSMITS146E_1010 | 497 |
| METSMITS146E | METSMITS146E_1046 | 498 |
| METSMITS146E | METSMITS146E_1085 | 499 |
| METSMITS146E | METSMITS146E_1172 | 500 |
| METSMITS146E | METSMITS146E_1206 | 501 |
| METSMITS146E | METSMITS146E_1207 | 502 |
| METSMITS146E | METSMITS146E_1208 | 503 |
| METSMITS146E | METSMITS146E_1209 | 504 |
| METSMITS146E | METSMITS146E_1210 | 505 |
| METSMITS146E | METSMITS146E_1211 | 506 |
| METSMITS146E | METSMITS146E_1268 | 507 |
| METSMITS146E | METSMITS146E_1273 | 508 |
| METSMITS146E | METSMITS146E_1390 | 509 |
| METSMITS146E | METSMITS146E_1391 | 510 |
| METSMITS146E | METSMITS146E_1392 | 511 |
| METSMITS146E | METSMITS146E_1417 | 512 |
| METSMITS146E | METSMITS146E_1418 | 513 |
| METSMITS146E | METSMITS146E_1502 | 514 |
| METSMITS146E | METSMITS146E_1569 | 515 |
| METSMITS146E | METSMITS146E_1624 | 516 |
| METSMITS146E | METSMITS146E_1648 | 517 |
| METSMITS146E | METSMITS146E_1660 | 518 |
| METSMITS146E | METSMITS146E_1677 | 519 |
| METSMITS146E | METSMITS146E_1678 | 520 |
| METSMITS146E | METSMITS146E_1679 | 521 |
| METSMITS146E | METSMITS146E_1779 | 522 |
| METSMITS146E | METSMITS146E_1782 | 523 |
| METSMITS146E | METSMITS146E_1862 | 524 |
| METSMITS146E | METSMITS146E_1866 | 525 |
| METSMITS147A | METSMITS147A_0012 | 526 |
| METSMITS147A | METSMITS147A_0033 | 527 |
| METSMITS147A | METSMITS147A_0039 | 528 |
| METSMITS147A | METSMITS147A_0076 | 529 |
| METSMITS147A | METSMITS147A_0158 | 530 |
| METSMITS147A | METSMITS147A_0207 | 531 |
| METSMITS147A | METSMITS147A_0209 | 532 |
| METSMITS147A | METSMITS147A_0220 | 533 |
| METSMITS147A | METSMITS147A_0258 | 534 |
| METSMITS147A | METSMITS147A_0259 | 535 |
| METSMITS147A | METSMITS147A_0275 | 536 |
| METSMITS147A | METSMITS147A_0360 | 537 |
| METSMITS147A | METSMITS147A_0407 | 538 |
| METSMITS147A | METSMITS147A_0408 | 539 |
| METSMITS147A | METSMITS147A_0747 | 540 |
| METSMITS147A | METSMITS147A_0748 | 541 |
| METSMITS147A | METSMITS147A_0749 | 542 |
| METSMITS147A | METSMITS147A_0751 | 543 |
| METSMITS147A | METSMITS147A_0854 | 544 |
| METSMITS147A | METSMITS147A_0961 | 545 |
| METSMITS147A | METSMITS147A_0997 | 546 |
| METSMITS147A | METSMITS147A_0998 | 547 |
| METSMITS147A | METSMITS147A_1037 | 548 |
| METSMITS147A | METSMITS147A_1075 | 549 |
| METSMITS147A | METSMITS147A_1161 | 550 |
| METSMITS147A | METSMITS147A_1196 | 551 |
| METSMITS147A | METSMITS147A_1197 | 552 |
| METSMITS147A | METSMITS147A_1198 | 553 |
| METSMITS147A | METSMITS147A_1199 | 554 |
| METSMITS147A | METSMITS147A_1200 | 555 |
| METSMITS147A | METSMITS147A_1201 | 556 |
| METSMITS147A | METSMITS147A_1258 | 557 |
| METSMITS147A | METSMITS147A_1263 | 558 |
| METSMITS147A | METSMITS147A_1431 | 559 |
| METSMITS147A | METSMITS147A_1432 | 560 |
| METSMITS147A | METSMITS147A_1458 | 561 |
| METSMITS147A | METSMITS147A_1538 | 562 |
| METSMITS147A | METSMITS147A_1605 | 563 |
| METSMITS147A | METSMITS147A_1671 | 564 |
| METSMITS147A | METSMITS147A_1672 | 565 |
| METSMITS147A | METSMITS147A_1696 | 566 |
| METSMITS147A | METSMITS147A_1709 | 567 |
| METSMITS147A | METSMITS147A_1710 | 568 |
| METSMITS147A | METSMITS147A_1727 | 569 |
| METSMITS147A | METSMITS147A_1728 | 570 |
| METSMITS147A | METSMITS147A_1840 | 571 |
| METSMITS147A | METSMITS147A_1844 | 572 |
| METSMITS147A | METSMITS147A_1954 | 573 |
| METSMITS147A | METSMITS147A_1955 | 574 |
| METSMITS147A | METSMITS147A_1965 | 575 |
| METSMITS147A | METSMITS147A_1966 | 576 |
| METSMITS147B | METSMITS147B_0020 | 577 |
| METSMITS147B | METSMITS147B_0040 | 578 |
| METSMITS147B | METSMITS147B_0041 | 579 |
| METSMITS147B | METSMITS147B_0047 | 580 |
| METSMITS147B | METSMITS147B_0083 | 581 |
| METSMITS147B | METSMITS147B_0165 | 582 |
| METSMITS147B | METSMITS147B_0212 | 583 |
| METSMITS147B | METSMITS147B_0214 | 584 |
| METSMITS147B | METSMITS147B_0225 | 585 |
| METSMITS147B | METSMITS147B_0226 | 586 |
| METSMITS147B | METSMITS147B_0227 | 587 |
| METSMITS147B | METSMITS147B_0275 | 588 |
| METSMITS147B | METSMITS147B_0291 | 589 |
| METSMITS147B | METSMITS147B_0377 | 590 |
| METSMITS147B | METSMITS147B_0424 | 591 |
| METSMITS147B | METSMITS147B_0425 | 592 |
| METSMITS147B | METSMITS147B_0608 | 593 |
| METSMITS147B | METSMITS147B_0609 | 594 |
| METSMITS147B | METSMITS147B_0610 | 595 |
| METSMITS147B | METSMITS147B_0899 | 596 |
| METSMITS147B | METSMITS147B_0900 | 597 |
| METSMITS147B | METSMITS147B_1017 | 598 |
| METSMITS147B | METSMITS147B_1052 | 599 |
| METSMITS147B | METSMITS147B_1053 | 600 |
| METSMITS147B | METSMITS147B_1089 | 601 |
| METSMITS147B | METSMITS147B_1128 | 602 |
| METSMITS147B | METSMITS147B_1129 | 603 |
| METSMITS147B | METSMITS147B_1130 | 604 |
| METSMITS147B | METSMITS147B_1216 | 605 |
| METSMITS147B | METSMITS147B_1251 | 606 |
| METSMITS147B | METSMITS147B_1252 | 607 |
| METSMITS147B | METSMITS147B_1253 | 608 |
| METSMITS147B | METSMITS147B_1254 | 609 |
| METSMITS147B | METSMITS147B_1255 | 610 |
| METSMITS147B | METSMITS147B_1256 | 611 |
| METSMITS147B | METSMITS147B_1313 | 612 |
| METSMITS147B | METSMITS147B_1318 | 613 |
| METSMITS147B | METSMITS147B_1435 | 614 |
| METSMITS147B | METSMITS147B_1436 | 615 |
| METSMITS147B | METSMITS147B_1437 | 615 |
| METSMITS147B | METSMITS147B_1530 | 617 |
| METSMITS147B | METSMITS147B_1545 | 618 |
| METSMITS147B | METSMITS147B_1556 | 619 |
| METSMITS147B | METSMITS147B_1558 | 620 |
| METSMITS147B | METSMITS147B_1559 | 621 |
| METSMITS147B | METSMITS147B_1661 | 622 |
| METSMITS147B | METSMITS147B_1717 | 623 |
| METSMITS147B | METSMITS147B_1741 | 624 |
| METSMITS147B | METSMITS147B_1753 | 625 |
| METSMITS147B | METSMITS147B_1817 | 626 |
| METSMITS147B | METSMITS147B_1821 | 627 |
| METSMITS147B | METSMITS147B_1895 | 628 |
| METSMITS147B | METSMITS147B_1898 | 629 |
| METSMITS147C | METSMITS147C_0021 | 630 |
| METSMITS147C | METSMITS147C_0022 | 631 |
| METSMITS147C | METSMITS147C_0029 | 632 |
| METSMITS147C | METSMITS147C_0075 | 633 |
| METSMITS147C | METSMITS147C_0159 | 634 |
| METSMITS147C | METSMITS147C_0162 | 635 |
| METSMITS147C | METSMITS147C_0163 | 636 |
| METSMITS147C | METSMITS147C_0211 | 637 |
| METSMITS147C | METSMITS147C_0213 | 638 |
| METSMITS147C | METSMITS147C_0224 | 639 |
| METSMITS147C | METSMITS147C_0283 | 640 |
| METSMITS147C | METSMITS147C_0299 | 641 |
| METSMITS147C | METSMITS147C_0388 | 642 |
| METSMITS147C | METSMITS147C_0389 | 643 |
| METSMITS147C | METSMITS147C_0438 | 644 |
| METSMITS147C | METSMITS147C_0439 | 645 |
| METSMITS147C | METSMITS147C_0630 | 646 |
| METSMITS147C | METSMITS147C_0631 | 647 |
| METSMITS147C | METSMITS147C_0632 | 648 |
| METSMITS147C | METSMITS147C_0633 | 649 |
| METSMITS147C | METSMITS147C_0856 | 650 |
| METSMITS147C | METSMITS147C_0857 | 651 |
| METSMITS147C | METSMITS147C_0859 | 652 |
| METSMITS147C | METSMITS147C_0893 | 653 |
| METSMITS147C | METSMITS147C_0934 | 654 |
| METSMITS147C | METSMITS147C_0935 | 655 |
| METSMITS147C | METSMITS147C_0972 | 656 |
| METSMITS147C | METSMITS147C_1012 | 657 |
| METSMITS147C | METSMITS147C_1013 | 658 |
| METSMITS147C | METSMITS147C_1099 | 659 |
| METSMITS147C | METSMITS147C_1100 | 660 |
| METSMITS147C | METSMITS147C_1134 | 661 |
| METSMITS147C | METSMITS147C_1135 | 662 |
| METSMITS147C | METSMITS147C_1136 | 663 |
| METSMITS147C | METSMITS147C_1137 | 664 |
| METSMITS147C | METSMITS147C_1138 | 665 |
| METSMITS147C | METSMITS147C_1139 | 666 |
| METSMITS147C | METSMITS147C_1140 | 667 |
| METSMITS147C | METSMITS147C_1141 | 668 |
| METSMITS147C | METSMITS147C_1197 | 669 |
| METSMITS147C | METSMITS147C_1201 | 670 |
| METSMITS147C | METSMITS147C_1318 | 671 |
| METSMITS147C | METSMITS147C_1319 | 672 |
| METSMITS147C | METSMITS147C_1433 | 673 |
| METSMITS147C | METSMITS147C_1524 | 674 |
| METSMITS147C | METSMITS147C_1695 | 675 |
| METSMITS147C | METSMITS147C_1751 | 676 |
| METSMITS147C | METSMITS147C_1775 | 677 |
| METSMITS147C | METSMITS147C_1856 | 678 |
| METSMITS147C | METSMITS147C_1860 | 679 |
| METSMITS147C | METSMITS147C_1965 | 680 |
| METSMITS147C | METSMITS147C_1978 | 681 |
| METSMITS147C | METSMITS147C_2005 | 682 |
| METSMITS94A | METSMITS94A_0032 | 683 |
| METSMITS94A | METSMITS94A_0162 | 684 |
| METSMITS94A | METSMITS94A_0164 | 685 |
| METSMITS94A | METSMITS94A_0209 | 686 |
| METSMITS94A | METSMITS94A_0220 | 687 |
| METSMITS94A | METSMITS94A_0221 | 688 |
| METSMITS94A | METSMITS94A_0232 | 689 |
| METSMITS94A | METSMITS94A_0312 | 690 |
| METSMITS94A | METSMITS94A_0358 | 691 |
| METSMITS94A | METSMITS94A_0359 | 692 |
| METSMITS94A | METSMITS94A_0409 | 693 |
| METSMITS94A | METSMITS94A_0713 | 694 |
| METSMITS94A | METSMITS94A_0714 | 695 |
| METSMITS94A | METSMITS94A_0716 | 696 |
| METSMITS94A | METSMITS94A_0730 | 697 |
| METSMITS94A | METSMITS94A_0731 | 698 |
| METSMITS94A | METSMITS94A_0732 | 699 |
| METSMITS94A | METSMITS94A_0733 | 700 |
| METSMITS94A | METSMITS94A_0796 | 701 |
| METSMITS94A | METSMITS94A_0898 | 702 |
| METSMITS94A | METSMITS94A_0933 | 703 |
| METSMITS94A | METSMITS94A_0934 | 704 |
| METSMITS94A | METSMITS94A_0971 | 705 |
| METSMITS94A | METSMITS94A_1009 | 706 |
| METSMITS94A | METSMITS94A_1128 | 707 |
| METSMITS94A | METSMITS94A_1129 | 708 |
| METSMITS94A | METSMITS94A_1130 | 709 |
| METSMITS94A | METSMITS94A_1131 | 710 |
| METSMITS94A | METSMITS94A_1163 | 711 |
| METSMITS94A | METSMITS94A_1195 | 712 |
| METSMITS94A | METSMITS94A_1199 | 713 |
| METSMITS94A | METSMITS94A_1322 | 714 |
| METSMITS94A | METSMITS94A_1323 | 715 |
| METSMITS94A | METSMITS94A_1348 | 716 |
| METSMITS94A | METSMITS94A_1429 | 717 |
| METSMITS94A | METSMITS94A_1430 | 718 |
| METSMITS94A | METSMITS94A_1431 | 719 |
| METSMITS94A | METSMITS94A_1501 | 720 |
| METSMITS94A | METSMITS94A_1559 | 721 |
| METSMITS94A | METSMITS94A_1589 | 722 |
| METSMITS94A | METSMITS94A_1603 | 723 |
| METSMITS94A | METSMITS94A_1622 | 724 |
| METSMITS94A | METSMITS94A_1623 | 725 |
| METSMITS94A | METSMITS94A_1624 | 726 |
| METSMITS94A | METSMITS94A_1625 | 727 |
| METSMITS94A | METSMITS94A_1722 | 728 |
| METSMITS94A | METSMITS94A_1725 | 729 |
| METSMITS94A | METSMITS94A_1766 | 730 |
| METSMITS94A | METSMITS94A_1786 | 731 |
| METSMITS94A | METSMITS94A_1787 | 732 |
| METSMITS94A | METSMITS94A_1790 | 733 |
| METSMITS94A | METSMITS94A_1796 | 734 |
| METSMITS94B | METSMITS94B_0013 | 735 |
| METSMITS94B | METSMITS94B_0146 | 736 |
| METSMITS94B | METSMITS94B_0148 | 737 |
| METSMITS94B | METSMITS94B_0157 | 738 |
| METSMITS94B | METSMITS94B_0158 | 739 |
| METSMITS94B | METSMITS94B_0159 | 740 |
| METSMITS94B | METSMITS94B_0166 | 741 |
| METSMITS94B | METSMITS94B_0167 | 742 |
| METSMITS94B | METSMITS94B_0207 | 743 |
| METSMITS94B | METSMITS94B_0219 | 744 |
| METSMITS94B | METSMITS94B_0220 | 745 |
| METSMITS94B | METSMITS94B_0231 | 746 |
| METSMITS94B | METSMITS94B_0312 | 747 |
| METSMITS94B | METSMITS94B_0359 | 748 |
| METSMITS94B | METSMITS94B_0360 | 749 |
| METSMITS94B | METSMITS94B_0411 | 750 |
| METSMITS94B | METSMITS94B_0412 | 751 |
| METSMITS94B | METSMITS94B_0719 | 752 |
| METSMITS94B | METSMITS94B_0720 | 753 |
| METSMITS94B | METSMITS94B_0721 | 754 |
| METSMITS94B | METSMITS94B_0724 | 755 |
| METSMITS94B | METSMITS94B_0725 | 756 |
| METSMITS94B | METSMITS94B_0726 | 757 |
| METSMITS94B | METSMITS94B_0788 | 758 |
| METSMITS94B | METSMITS94B_0892 | 759 |
| METSMITS94B | METSMITS94B_0927 | 760 |
| METSMITS94B | METSMITS94B_0928 | 761 |
| METSMITS94B | METSMITS94B_0966 | 762 |
| METSMITS94B | METSMITS94B_1130 | 763 |
| METSMITS94B | METSMITS94B_1131 | 764 |
| METSMITS94B | METSMITS94B_1132 | 765 |
| METSMITS94B | METSMITS94B_1133 | 766 |
| METSMITS94B | METSMITS94B_1134 | 767 |
| METSMITS94B | METSMITS94B_1166 | 768 |
| METSMITS94B | METSMITS94B_1197 | 769 |
| METSMITS94B | METSMITS94B_1201 | 770 |
| METSMITS94B | METSMITS94B_1328 | 771 |
| METSMITS94B | METSMITS94B_1329 | 772 |
| METSMITS94B | METSMITS94B_1353 | 773 |
| METSMITS94B | METSMITS94B_1354 | 774 |
| METSMITS94B | METSMITS94B_1446 | 775 |
| METSMITS94B | METSMITS94B_1517 | 776 |
| METSMITS94B | METSMITS94B_1579 | 777 |
| METSMITS94B | METSMITS94B_1611 | 778 |
| METSMITS94B | METSMITS94B_1612 | 780 |
| METSMITS94B | METSMITS94B_1627 | 781 |
| METSMITS94B | METSMITS94B_1648 | 782 |
| METSMITS94B | METSMITS94B_1649 | 783 |
| METSMITS94B | METSMITS94B_1650 | 784 |
| METSMITS94B | METSMITS94B_1651 | 785 |
| METSMITS94B | METSMITS94B_1752 | 786 |
| METSMITS94B | METSMITS94B_1755 | 787 |
| METSMITS94B | METSMITS94B_1797 | 788 |
| METSMITS94B | METSMITS94B_1818 | 789 |
| METSMITS94B | METSMITS94B_1819 | 790 |
| METSMITS94B | METSMITS94B_1822 | 791 |
| METSMITS94B | METSMITS94B_1829 | 792 |
| METSMITS94C | METSMITS94C_0005 | 793 |
| METSMITS94C | METSMITS94C_0041 | 794 |
| METSMITS94C | METSMITS94C_0169 | 795 |
| METSMITS94C | METSMITS94C_0171 | 796 |
| METSMITS94C | METSMITS94C_0180 | 797 |
| METSMITS94C | METSMITS94C_0216 | 798 |
| METSMITS94C | METSMITS94C_0228 | 799 |
| METSMITS94C | METSMITS94C_0229 | 800 |
| METSMITS94C | METSMITS94C_0240 | 801 |
| METSMITS94C | METSMITS94C_0320 | 802 |
| METSMITS94C | METSMITS94C_0321 | 803 |
| METSMITS94C | METSMITS94C_0367 | 804 |
| METSMITS94C | METSMITS94C_0368 | 805 |
| METSMITS94C | METSMITS94C_0419 | 806 |
| METSMITS94C | METSMITS94C_0716 | 807 |
| METSMITS94C | METSMITS94C_0717 | 808 |
| METSMITS94C | METSMITS94C_0719 | 809 |
| METSMITS94C | METSMITS94C_0783 | 810 |
| METSMITS94C | METSMITS94C_0884 | 811 |
| METSMITS94C | METSMITS94C_0918 | 812 |
| METSMITS94C | METSMITS94C_0919 | 813 |
| METSMITS94C | METSMITS94C_0956 | 814 |
| METSMITS94C | METSMITS94C_1115 | 815 |
| METSMITS94C | METSMITS94C_1116 | 816 |
| METSMITS94C | METSMITS94C_1117 | 817 |
| METSMITS94C | METSMITS94C_1118 | 818 |
| METSMITS94C | METSMITS94C_1154 | 819 |
| METSMITS94C | METSMITS94C_1155 | 820 |
| METSMITS94C | METSMITS94C_1186 | 821 |
| METSMITS94C | METSMITS94C_1190 | 822 |
| METSMITS94C | METSMITS94C_1318 | 823 |
| METSMITS94C | METSMITS94C_1319 | 824 |
| METSMITS94C | METSMITS94C_1320 | 825 |
| METSMITS94C | METSMITS94C_1344 | 826 |
| METSMITS94C | METSMITS94C_1427 | 827 |
| METSMITS94C | METSMITS94C_1428 | 828 |
| METSMITS94C | METSMITS94C_1429 | 829 |
| METSMITS94C | METSMITS94C_1430 | 830 |
| METSMITS94C | METSMITS94C_1507 | 831 |
| METSMITS94C | METSMITS94C_1563 | 832 |
| METSMITS94C | METSMITS94C_1585 | 833 |
| METSMITS94C | METSMITS94C_1597 | 834 |
| METSMITS94C | METSMITS94C_1615 | 835 |
| METSMITS94C | METSMITS94C_1616 | 836 |
| METSMITS94C | METSMITS94C_1617 | 837 |
| METSMITS94C | METSMITS94C_1715 | 838 |
| METSMITS94C | METSMITS94C_1718 | 839 |
| METSMITS94C | METSMITS94C_1759 | 840 |
| METSMITS94C | METSMITS94C_1779 | 841 |
| METSMITS94C | METSMITS94C_1780 | 842 |
| METSMITS94C | METSMITS94C_1783 | 843 |
| METSMITS94C | METSMITS94C_1794 | 844 |
| METSMITS95A | METSMITS95A_0027 | 845 |
| METSMITS95A | METSMITS95A_0049 | 846 |
| METSMITS95A | METSMITS95A_0050 | 847 |
| METSMITS95A | METSMITS95A_0052 | 848 |
| METSMITS95A | METSMITS95A_0057 | 849 |
| METSMITS95A | METSMITS95A_0095 | 850 |
| METSMITS95A | METSMITS95A_0186 | 851 |
| METSMITS95A | METSMITS95A_0187 | 852 |
| METSMITS95A | METSMITS95A_0234 | 853 |
| METSMITS95A | METSMITS95A_0235 | 854 |
| METSMITS95A | METSMITS95A_0237 | 855 |
| METSMITS95A | METSMITS95A_0285 | 856 |
| METSMITS95A | METSMITS95A_0297 | 857 |
| METSMITS95A | METSMITS95A_0309 | 858 |
| METSMITS95A | METSMITS95A_0418 | 859 |
| METSMITS95A | METSMITS95A_0466 | 860 |
| METSMITS95A | METSMITS95A_0467 | 861 |
| METSMITS95A | METSMITS95A_0769 | 862 |
| METSMITS95A | METSMITS95A_0770 | 863 |
| METSMITS95A | METSMITS95A_0771 | 864 |
| METSMITS95A | METSMITS95A_0772 | 865 |
| METSMITS95A | METSMITS95A_0773 | 866 |
| METSMITS95A | METSMITS95A_0774 | 867 |
| METSMITS95A | METSMITS95A_0775 | 868 |
| METSMITS95A | METSMITS95A_0840 | 869 |
| METSMITS95A | METSMITS95A_0945 | 870 |
| METSMITS95A | METSMITS95A_0982 | 871 |
| METSMITS95A | METSMITS95A_0983 | 872 |
| METSMITS95A | METSMITS95A_1021 | 873 |
| METSMITS95A | METSMITS95A_1064 | 874 |
| METSMITS95A | METSMITS95A_1065 | 875 |
| METSMITS95A | METSMITS95A_1159 | 876 |
| METSMITS95A | METSMITS95A_1195 | 877 |
| METSMITS95A | METSMITS95A_1196 | 878 |
| METSMITS95A | METSMITS95A_1197 | 879 |
| METSMITS95A | METSMITS95A_1198 | 880 |
| METSMITS95A | METSMITS95A_1199 | 881 |
| METSMITS95A | METSMITS95A_1200 | 882 |
| METSMITS95A | METSMITS95A_1234 | 883 |
| METSMITS95A | METSMITS95A_1235 | 884 |
| METSMITS95A | METSMITS95A_1269 | 885 |
| METSMITS95A | METSMITS95A_1273 | 886 |
| METSMITS95A | METSMITS95A_1406 | 887 |
| METSMITS95A | METSMITS95A_1407 | 888 |
| METSMITS95A | METSMITS95A_1432 | 889 |
| METSMITS95A | METSMITS95A_1443 | 890 |
| METSMITS95A | METSMITS95A_1447 | 891 |
| METSMITS95A | METSMITS95A_1448 | 892 |
| METSMITS95A | METSMITS95A_1487 | 893 |
| METSMITS95A | METSMITS95A_1571 | 894 |
| METSMITS95A | METSMITS95A_1646 | 895 |
| METSMITS95A | METSMITS95A_1727 | 896 |
| METSMITS95A | METSMITS95A_1728 | 897 |
| METSMITS95A | METSMITS95A_1744 | 898 |
| METSMITS95A | METSMITS95A_1762 | 899 |
| METSMITS95A | METSMITS95A_1763 | 900 |
| METSMITS95A | METSMITS95A_1764 | 901 |
| METSMITS95A | METSMITS95A_1765 | 902 |
| METSMITS95A | METSMITS95A_1766 | 903 |
| METSMITS95A | METSMITS95A_1767 | 904 |
| METSMITS95A | METSMITS95A_1770 | 905 |
| METSMITS95A | METSMITS95A_1771 | 906 |
| METSMITS95A | METSMITS95A_1772 | 907 |
| METSMITS95A | METSMITS95A_1857 | 908 |
| METSMITS95A | METSMITS95A_1877 | 909 |
| METSMITS95A | METSMITS95A_1878 | 910 |
| METSMITS95A | METSMITS95A_1882 | 911 |
| METSMITS95A | METSMITS95A_1953 | 912 |
| METSMITS95A | METSMITS95A_1954 | 913 |
| METSMITS95A | METSMITS95A_1956 | 914 |
| METSMITS95A | METSMITS95A_1960 | 915 |
| METSMITS95B | METSMITS95B_0044 | 916 |
| METSMITS95B | METSMITS95B_0045 | 917 |
| METSMITS95B | METSMITS95B_0047 | 918 |
| METSMITS95B | METSMITS95B_0054 | 919 |
| METSMITS95B | METSMITS95B_0089 | 920 |
| METSMITS95B | METSMITS95B_0182 | 921 |
| METSMITS95B | METSMITS95B_0183 | 922 |
| METSMITS95B | METSMITS95B_0232 | 923 |
| METSMITS95B | METSMITS95B_0234 | 924 |
| METSMITS95B | METSMITS95B_0279 | 925 |
| METSMITS95B | METSMITS95B_0290 | 926 |
| METSMITS95B | METSMITS95B_0302 | 927 |
| METSMITS95B | METSMITS95B_0409 | 928 |
| METSMITS95B | METSMITS95B_0456 | 929 |
| METSMITS95B | METSMITS95B_0457 | 930 |
| METSMITS95B | METSMITS95B_0458 | 931 |
| METSMITS95B | METSMITS95B_0758 | 932 |
| METSMITS95B | METSMITS95B_0759 | 933 |
| METSMITS95B | METSMITS95B_0760 | 934 |
| METSMITS95B | METSMITS95B_0761 | 935 |
| METSMITS95B | METSMITS95B_0823 | 936 |
| METSMITS95B | METSMITS95B_0929 | 937 |
| METSMITS95B | METSMITS95B_0968 | 938 |
| METSMITS95B | METSMITS95B_0969 | 939 |
| METSMITS95B | METSMITS95B_0970 | 940 |
| METSMITS95B | METSMITS95B_1007 | 941 |
| METSMITS95B | METSMITS95B_1046 | 942 |
| METSMITS95B | METSMITS95B_1134 | 943 |
| METSMITS95B | METSMITS95B_1169 | 944 |
| METSMITS95B | METSMITS95B_1170 | 945 |
| METSMITS95B | METSMITS95B_1171 | 946 |
| METSMITS95B | METSMITS95B_1172 | 947 |
| METSMITS95B | METSMITS95B_1173 | 948 |
| METSMITS95B | METSMITS95B_1234 | 949 |
| METSMITS95B | METSMITS95B_1238 | 950 |
| METSMITS95B | METSMITS95B_1365 | 951 |
| METSMITS95B | METSMITS95B_1366 | 952 |
| METSMITS95B | METSMITS95B_1476 | 953 |
| METSMITS95B | METSMITS95B_1487 | 954 |
| METSMITS95B | METSMITS95B_1489 | 955 |
| METSMITS95B | METSMITS95B_1490 | 956 |
| METSMITS95B | METSMITS95B_1601 | 957 |
| METSMITS95B | METSMITS95B_1665 | 958 |
| METSMITS95B | METSMITS95B_1679 | 959 |
| METSMITS95B | METSMITS95B_1697 | 960 |
| METSMITS95B | METSMITS95B_1698 | 961 |
| METSMITS95B | METSMITS95B_1699 | 962 |
| METSMITS95B | METSMITS95B_1701 | 963 |
| METSMITS95B | METSMITS95B_1702 | 964 |
| METSMITS95B | METSMITS95B_1703 | 965 |
| METSMITS95B | METSMITS95B_1706 | 966 |
| METSMITS95B | METSMITS95B_1707 | 967 |
| METSMITS95B | METSMITS95B_1708 | 968 |
| METSMITS95B | METSMITS95B_1781 | 969 |
| METSMITS95B | METSMITS95B_1802 | 970 |
| METSMITS95B | METSMITS95B_1806 | 971 |
| METSMITS95B | METSMITS95B_1890 | 972 |
| METSMITS95B | METSMITS95B_1894 | 973 |
| METSMITS95C | METSMITS95C_0085 | 974 |
| METSMITS95C | METSMITS95C_0104 | 975 |
| METSMITS95C | METSMITS95C_0105 | 976 |
| METSMITS95C | METSMITS95C_0107 | 977 |
| METSMITS95C | METSMITS95C_0112 | 978 |
| METSMITS95C | METSMITS95C_0150 | 979 |
| METSMITS95C | METSMITS95C_0242 | 980 |
| METSMITS95C | METSMITS95C_0289 | 981 |
| METSMITS95C | METSMITS95C_0291 | 982 |
| METSMITS95C | METSMITS95C_0336 | 983 |
| METSMITS95C | METSMITS95C_0348 | 984 |
| METSMITS95C | METSMITS95C_0358 | 985 |
| METSMITS95C | METSMITS95C_0464 | 986 |
| METSMITS95C | METSMITS95C_0510 | 987 |
| METSMITS95C | METSMITS95C_0511 | 988 |
| METSMITS95C | METSMITS95C_0811 | 989 |
| METSMITS95C | METSMITS95C_0812 | 990 |
| METSMITS95C | METSMITS95C_0813 | 991 |
| METSMITS95C | METSMITS95C_0814 | 992 |
| METSMITS95C | METSMITS95C_0875 | 993 |
| METSMITS95C | METSMITS95C_0981 | 994 |
| METSMITS95C | METSMITS95C_1019 | 995 |
| METSMITS95C | METSMITS95C_1020 | 996 |
| METSMITS95C | METSMITS95C_1056 | 997 |
| METSMITS95C | METSMITS95C_1095 | 998 |
| METSMITS95C | METSMITS95C_1180 | 999 |
| METSMITS95C | METSMITS95C_1215 | 1000 |
| METSMITS95C | METSMITS95C_1216 | 1001 |
| METSMITS95C | METSMITS95C_1217 | 1002 |
| METSMITS95C | METSMITS95C_1218 | 1003 |
| METSMITS95C | METSMITS95C_1246 | 1004 |
| METSMITS95C | METSMITS95C_1278 | 1005 |
| METSMITS95C | METSMITS95C_1282 | 1006 |
| METSMITS95C | METSMITS95C_1407 | 1007 |
| METSMITS95C | METSMITS95C_1408 | 1008 |
| METSMITS95C | METSMITS95C_1516 | 1009 |
| METSMITS95C | METSMITS95C_1527 | 1010 |
| METSMITS95C | METSMITS95C_1529 | 1011 |
| METSMITS95C | METSMITS95C_1530 | 1012 |
| METSMITS95C | METSMITS95C_1640 | 1013 |
| METSMITS95C | METSMITS95C_1713 | 1014 |
| METSMITS95C | METSMITS95C_1727 | 1015 |
| METSMITS95C | METSMITS95C_1732 | 1016 |
| METSMITS95C | METSMITS95C_1751 | 1017 |
| METSMITS95C | METSMITS95C_1752 | 1018 |
| METSMITS95C | METSMITS95C_1753 | 1019 |
| METSMITS95C | METSMITS95C_1754 | 1020 |
| METSMITS95C | METSMITS95C_1755 | 1021 |
| METSMITS95C | METSMITS95C_1757 | 1022 |
| METSMITS95C | METSMITS95C_1758 | 1023 |
| METSMITS95C | METSMITS95C_1837 | 1024 |
| METSMITS95C | METSMITS95C_1857 | 1025 |
| METSMITS95C | METSMITS95C_1861 | 1026 |
| METSMITS95C | METSMITS95C_1874 | 1027 |
| METSMITS95D | METSMITS95D_0029 | 1028 |
| METSMITS95D | METSMITS95D_0050 | 1029 |
| METSMITS95D | METSMITS95D_0051 | 1030 |
| METSMITS95D | METSMITS95D_0052 | 1031 |
| METSMITS95D | METSMITS95D_0053 | 1032 |
| METSMITS95D | METSMITS95D_0055 | 1033 |
| METSMITS95D | METSMITS95D_0060 | 1034 |
| METSMITS95D | METSMITS95D_0097 | 1035 |
| METSMITS95D | METSMITS95D_0238 | 1036 |
| METSMITS95D | METSMITS95D_0240 | 1037 |
| METSMITS95D | METSMITS95D_0285 | 1038 |
| METSMITS95D | METSMITS95D_0296 | 1039 |
| METSMITS95D | METSMITS95D_0307 | 1040 |
| METSMITS95D | METSMITS95D_0411 | 1041 |
| METSMITS95D | METSMITS95D_0412 | 1042 |
| METSMITS95D | METSMITS95D_0458 | 1043 |
| METSMITS95D | METSMITS95D_0459 | 1044 |
| METSMITS95D | METSMITS95D_0726 | 1045 |
| METSMITS95D | METSMITS95D_0727 | 1046 |
| METSMITS95D | METSMITS95D_0728 | 1047 |
| METSMITS95D | METSMITS95D_0729 | 1048 |
| METSMITS95D | METSMITS95D_0730 | 1049 |
| METSMITS95D | METSMITS95D_0790 | 1050 |
| METSMITS95D | METSMITS95D_0892 | 1051 |
| METSMITS95D | METSMITS95D_0927 | 1052 |
| METSMITS95D | METSMITS95D_0928 | 1053 |
| METSMITS95D | METSMITS95D_0964 | 1054 |
| METSMITS95D | METSMITS95D_1003 | 1055 |
| METSMITS95D | METSMITS95D_1089 | 1056 |
| METSMITS95D | METSMITS95D_1123 | 1057 |
| METSMITS95D | METSMITS95D_1124 | 1058 |
| METSMITS95D | METSMITS95D_1125 | 1059 |
| METSMITS95D | METSMITS95D_1126 | 1060 |
| METSMITS95D | METSMITS95D_1127 | 1061 |
| METSMITS95D | METSMITS95D_1129 | 1062 |
| METSMITS95D | METSMITS95D_1130 | 1063 |
| METSMITS95D | METSMITS95D_1131 | 1064 |
| METSMITS95D | METSMITS95D_1189 | 1065 |
| METSMITS95D | METSMITS95D_1193 | 1066 |
| METSMITS95D | METSMITS95D_1316 | 1067 |
| METSMITS95D | METSMITS95D_1317 | 1068 |
| METSMITS95D | METSMITS95D_1423 | 1069 |
| METSMITS95D | METSMITS95D_1433 | 1070 |
| METSMITS95D | METSMITS95D_1435 | 1071 |
| METSMITS95D | METSMITS95D_1436 | 1072 |
| METSMITS95D | METSMITS95D_1540 | 1073 |
| METSMITS95D | METSMITS95D_1619 | 1074 |
| METSMITS95D | METSMITS95D_1632 | 1075 |
| METSMITS95D | METSMITS95D_1633 | 1076 |
| METSMITS95D | METSMITS95D_1634 | 1077 |
| METSMITS95D | METSMITS95D_1636 | 1078 |
| METSMITS95D | METSMITS95D_1637 | 1079 |
| METSMITS95D | METSMITS95D_1654 | 1080 |
| METSMITS95D | METSMITS95D_1655 | 1081 |
| METSMITS95D | METSMITS95D_1656 | 1082 |
| METSMITS95D | METSMITS95D_1657 | 1083 |
| METSMITS95D | METSMITS95D_1731 | 1084 |
| METSMITS95D | METSMITS95D_1751 | 1085 |
| METSMITS95D | METSMITS95D_1755 | 1086 |
| METSMITS95D | METSMITS95D_1804 | 1087 |
| METSMITS95D | METSMITS95D_1859 | 1088 |
| METSMITS96A | METSMITS96A_0055 | 1089 |
| METSMITS96A | METSMITS96A_0074 | 1090 |
| METSMITS96A | METSMITS96A_0075 | 1091 |
| METSMITS96A | METSMITS96A_0077 | 1092 |
| METSMITS96A | METSMITS96A_0082 | 1093 |
| METSMITS96A | METSMITS96A_0118 | 1094 |
| METSMITS96A | METSMITS96A_0191 | 1095 |
| METSMITS96A | METSMITS96A_0238 | 1096 |
| METSMITS96A | METSMITS96A_0240 | 1097 |
| METSMITS96A | METSMITS96A_0285 | 1098 |
| METSMITS96A | METSMITS96A_0296 | 1099 |
| METSMITS96A | METSMITS96A_0307 | 1100 |
| METSMITS96A | METSMITS96A_0414 | 1101 |
| METSMITS96A | METSMITS96A_0460 | 1102 |
| METSMITS96A | METSMITS96A_0461 | 1103 |
| METSMITS96A | METSMITS96A_0802 | 1104 |
| METSMITS96A | METSMITS96A_0907 | 1105 |
| METSMITS96A | METSMITS96A_0957 | 1106 |
| METSMITS96A | METSMITS96A_0958 | 1107 |
| METSMITS96A | METSMITS96A_0994 | 1108 |
| METSMITS96A | METSMITS96A_1032 | 1109 |
| METSMITS96A | METSMITS96A_1033 | 1110 |
| METSMITS96A | METSMITS96A_1119 | 1111 |
| METSMITS96A | METSMITS96A_1153 | 1112 |
| METSMITS96A | METSMITS96A_1154 | 1113 |
| METSMITS96A | METSMITS96A_1155 | 1114 |
| METSMITS96A | METSMITS96A_1156 | 1115 |
| METSMITS96A | METSMITS96A_1159 | 1116 |
| METSMITS96A | METSMITS96A_1188 | 1117 |
| METSMITS96A | METSMITS96A_1219 | 1118 |
| METSMITS96A | METSMITS96A_1223 | 1119 |
| METSMITS96A | METSMITS96A_1347 | 1120 |
| METSMITS96A | METSMITS96A_1348 | 1121 |
| METSMITS96A | METSMITS96A_1349 | 1122 |
| METSMITS96A | METSMITS96A_1455 | 1123 |
| METSMITS96A | METSMITS96A_1466 | 1124 |
| METSMITS96A | METSMITS96A_1468 | 1125 |
| METSMITS96A | METSMITS96A_1469 | 1126 |
| METSMITS96A | METSMITS96A_1512 | 1127 |
| METSMITS96A | METSMITS96A_1513 | 1128 |
| METSMITS96A | METSMITS96A_1514 | 1129 |
| METSMITS96A | METSMITS96A_1515 | 1130 |
| METSMITS96A | METSMITS96A_1586 | 1131 |
| METSMITS96A | METSMITS96A_1662 | 1132 |
| METSMITS96A | METSMITS96A_1674 | 1133 |
| METSMITS96A | METSMITS96A_1675 | 1134 |
| METSMITS96A | METSMITS96A_1677 | 1135 |
| METSMITS96A | METSMITS96A_1678 | 1136 |
| METSMITS96A | METSMITS96A_1697 | 1137 |
| METSMITS96A | METSMITS96A_1698 | 1138 |
| METSMITS96A | METSMITS96A_1699 | 1139 |
| METSMITS96A | METSMITS96A_1779 | 1140 |
| METSMITS96A | METSMITS96A_1798 | 1141 |
| METSMITS96A | METSMITS96A_1802 | 1142 |
| METSMITS96A | METSMITS96A_1845 | 1143 |
| METSMITS96A | METSMITS96A_1852 | 1144 |
| METSMITS96B | METSMITS96B_0027 | 1145 |
| METSMITS96B | METSMITS96B_0032 | 1146 |
| METSMITS96B | METSMITS96B_0066 | 1147 |
| METSMITS96B | METSMITS96B_0148 | 1148 |
| METSMITS96B | METSMITS96B_0149 | 1149 |
| METSMITS96B | METSMITS96B_0213 | 1150 |
| METSMITS96B | METSMITS96B_0260 | 1151 |
| METSMITS96B | METSMITS96B_0262 | 1152 |
| METSMITS96B | METSMITS96B_0306 | 1153 |
| METSMITS96B | METSMITS96B_0317 | 1154 |
| METSMITS96B | METSMITS96B_0328 | 1155 |
| METSMITS96B | METSMITS96B_0420 | 1156 |
| METSMITS96B | METSMITS96B_0466 | 1157 |
| METSMITS96B | METSMITS96B_0467 | 1158 |
| METSMITS96B | METSMITS96B_0811 | 1159 |
| METSMITS96B | METSMITS96B_0853 | 1160 |
| METSMITS96B | METSMITS96B_0887 | 1161 |
| METSMITS96B | METSMITS96B_0888 | 1162 |
| METSMITS96B | METSMITS96B_0924 | 1163 |
| METSMITS96B | METSMITS96B_0963 | 1164 |
| METSMITS96B | METSMITS96B_1049 | 1165 |
| METSMITS96B | METSMITS96B_1081 | 1166 |
| METSMITS96B | METSMITS96B_1082 | 1167 |
| METSMITS96B | METSMITS96B_1083 | 1168 |
| METSMITS96B | METSMITS96B_1084 | 1169 |
| METSMITS96B | METSMITS96B_1114 | 1170 |
| METSMITS96B | METSMITS96B_1145 | 1171 |
| METSMITS96B | METSMITS96B_1149 | 1172 |
| METSMITS96B | METSMITS96B_1303 | 1173 |
| METSMITS96B | METSMITS96B_1304 | 1174 |
| METSMITS96B | METSMITS96B_1315 | 1175 |
| METSMITS96B | METSMITS96B_1317 | 1176 |
| METSMITS96B | METSMITS96B_1318 | 1177 |
| METSMITS96B | METSMITS96B_1429 | 1178 |
| METSMITS96B | METSMITS96B_1505 | 1179 |
| METSMITS96B | METSMITS96B_1517 | 1180 |
| METSMITS96B | METSMITS96B_1534 | 1181 |
| METSMITS96B | METSMITS96B_1535 | 1182 |
| METSMITS96B | METSMITS96B_1536 | 1183 |
| METSMITS96B | METSMITS96B_1537 | 1184 |
| METSMITS96B | METSMITS96B_1539 | 1185 |
| METSMITS96B | METSMITS96B_1614 | 1186 |
| METSMITS96B | METSMITS96B_1633 | 1187 |
| METSMITS96B | METSMITS96B_1637 | 1188 |
| METSMITS96B | METSMITS96B_1709 | 1189 |
| METSMITS96B | METSMITS96B_1710 | 1190 |
| METSMITS96B | METSMITS96B_1711 | 1191 |
| METSMITS96B | METSMITS96B_1712 | 1192 |
| METSMITS96B | METSMITS96B_1716 | 1193 |
| METSMITS96B | METSMITS96B_1723 | 1194 |
| METSMITS96C | METSMITS96C_0022 | 1195 |
| METSMITS96C | METSMITS96C_0042 | 1196 |
| METSMITS96C | METSMITS96C_0043 | 1197 |
| METSMITS96C | METSMITS96C_0044 | 1198 |
| METSMITS96C | METSMITS96C_0084 | 1199 |
| METSMITS96C | METSMITS96C_0204 | 1200 |
| METSMITS96C | METSMITS96C_0206 | 1201 |
| METSMITS96C | METSMITS96C_0216 | 1202 |
| METSMITS96C | METSMITS96C_0253 | 1203 |
| METSMITS96C | METSMITS96C_0254 | 1204 |
| METSMITS96C | METSMITS96C_0255 | 1205 |
| METSMITS96C | METSMITS96C_0273 | 1206 |
| METSMITS96C | METSMITS96C_0275 | 1207 |
| METSMITS96C | METSMITS96C_0418 | 1208 |
| METSMITS96C | METSMITS96C_0420 | 1209 |
| METSMITS96C | METSMITS96C_0581 | 1210 |
| METSMITS96C | METSMITS96C_0782 | 1211 |
| METSMITS96C | METSMITS96C_0878 | 1212 |
| METSMITS96C | METSMITS96C_0879 | 1213 |
| METSMITS96C | METSMITS96C_0911 | 1214 |
| METSMITS96C | METSMITS96C_0918 | 1215 |
| METSMITS96C | METSMITS96C_0955 | 1216 |
| METSMITS96C | METSMITS96C_0995 | 1217 |
| METSMITS96C | METSMITS96C_1123 | 1218 |
| METSMITS96C | METSMITS96C_1126 | 1219 |
| METSMITS96C | METSMITS96C_1145 | 1220 |
| METSMITS96C | METSMITS96C_1265 | 1221 |
| METSMITS96C | METSMITS96C_1266 | 1222 |
| METSMITS96C | METSMITS96C_1268 | 1223 |
| METSMITS96C | METSMITS96C_1287 | 1224 |
| METSMITS96C | METSMITS96C_1299 | 1225 |
| METSMITS96C | METSMITS96C_1323 | 1226 |
| METSMITS96C | METSMITS96C_1324 | 1227 |
| METSMITS96C | METSMITS96C_1366 | 1228 |
| METSMITS96C | METSMITS96C_1432 | 1229 |
| METSMITS96C | METSMITS96C_1491 | 1230 |
| METSMITS96C | METSMITS96C_1512 | 1231 |
| METSMITS96C | METSMITS96C_1631 | 1232 |
| METSMITS96C | METSMITS96C_1632 | 1233 |
| METSMITS96C | METSMITS96C_1723 | 1234 |
| METSMITS96C | METSMITS96C_1724 | 1235 |
| METSMITS96C | METSMITS96C_1725 | 1236 |
| METSMITS96C | METSMITS96C_1762 | 1237 |
| ATCC 35061 | ref\|NC_009515.1\|: c209454-204964 | 1273 |
| ATCC 35061 | ref\|NC_009515.1\|: c748934-745596 | 1282 |
| ATCC 35061 | ref\|NC_009515.1\|: c885328-884720 | 1285 |

TABLE 1
General features of the M. smithii genome compared to other sequenced Methanobacteriales

| | Methanobrevibacter | Methanosphaera | Methanothermobacter |
|---|---|---|---|
| | smithii | stadtmanae | thermoautotrophicus |
| Genome Size (bp) | 1,853,160 | 1,767,403 | 1,751,377 |
| G + C content (%) | 31 | 28 | 50 |
| Coding Regions (%) | 90 | 84 | 90 |
| Number of ORFs | 1795 | 1534 | 1869 |
| rRNA operons | 2 | 4 | 2 |
| tRNA genes | 34 | 40 | 39 |
| tRNA genes with intron | 1 | 1 | 3 |
| Transposases (remnants) | 2 (20) | 1 (2) | 0 |
| Insertion Sequences | 8 | 4 | 0 |
| Restriction Modification System | 2/6/1 | 3/2/1 | 3/0/0 |
| Subunits (Type I/II/III) | | | |
| Putative Prophage | Yes | No | No |

TABLE 2
Predicted proteome of M. smithii strain PS and conservation among other strains
and in the fecal microbiome of two healthy adults.

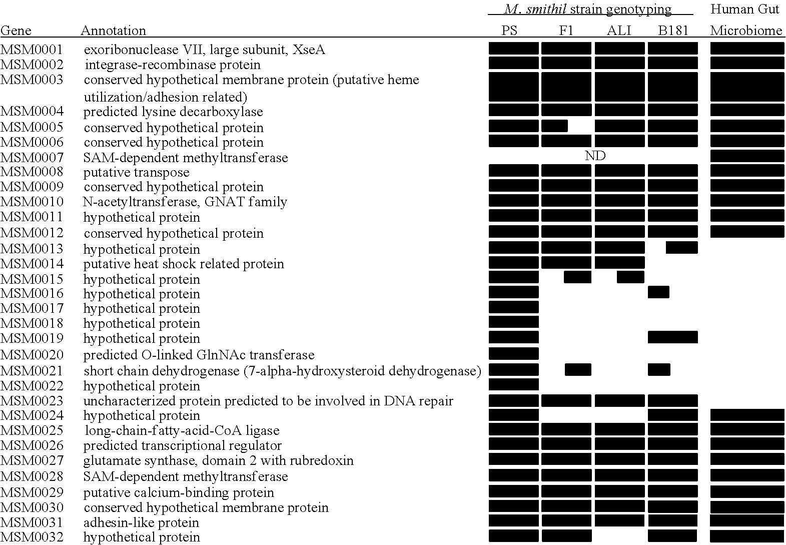

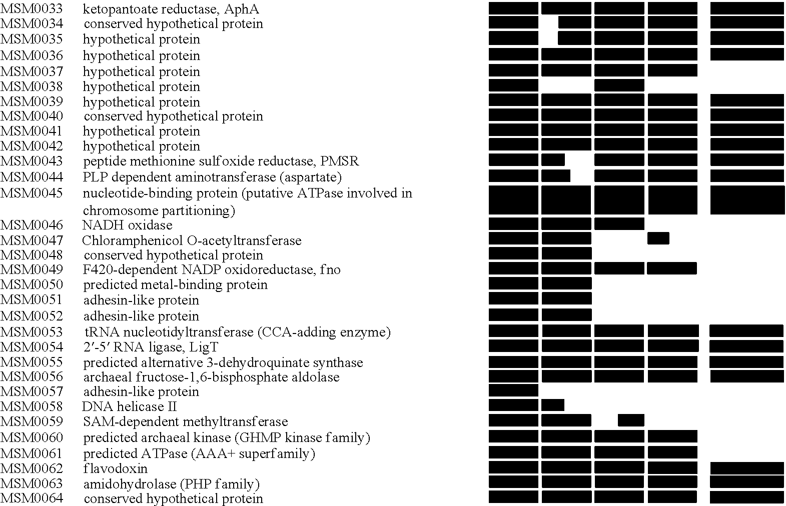

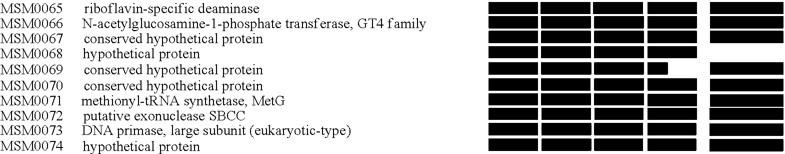

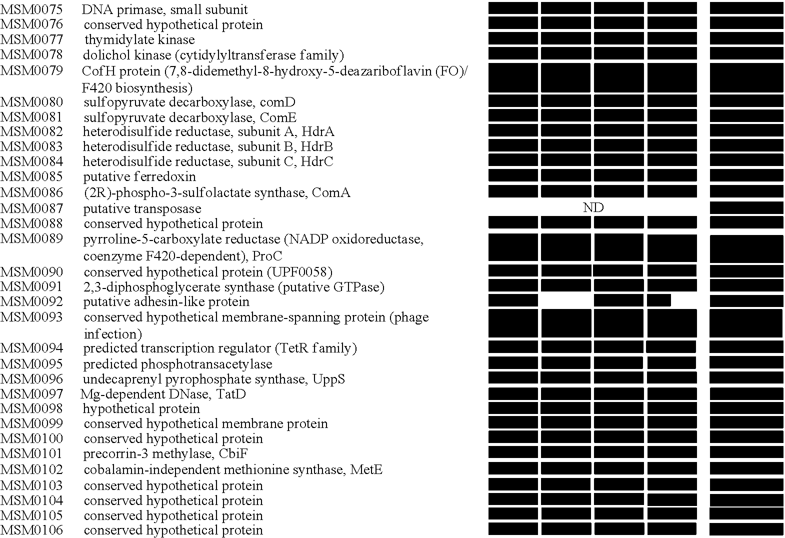

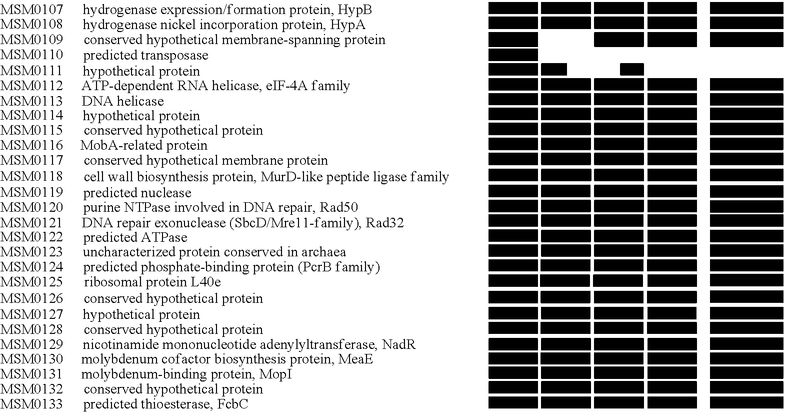

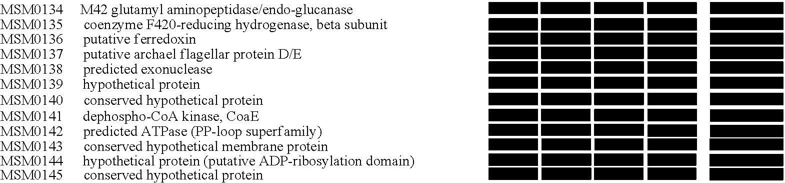

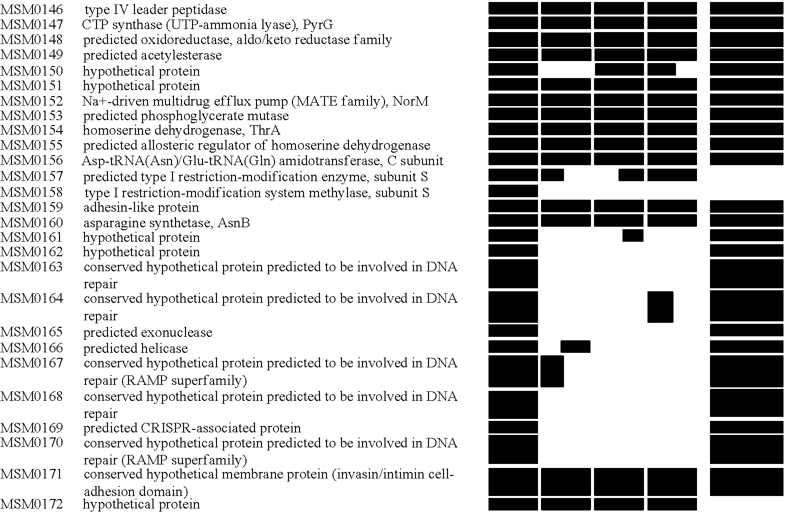

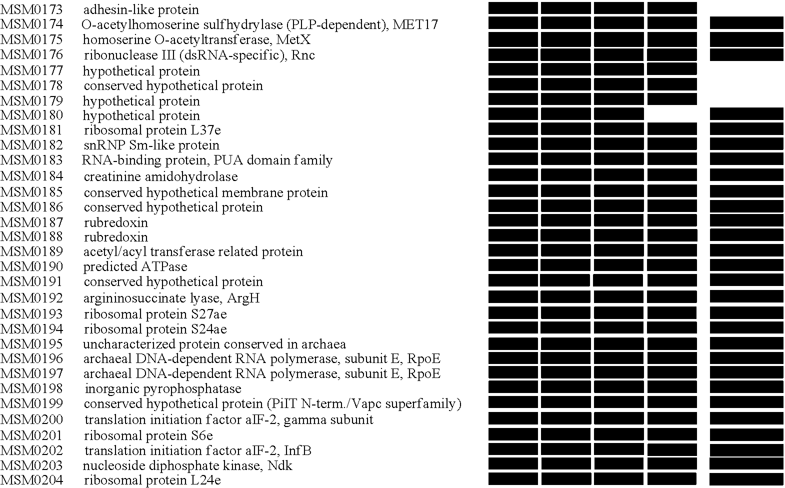

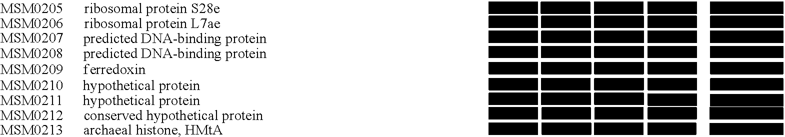

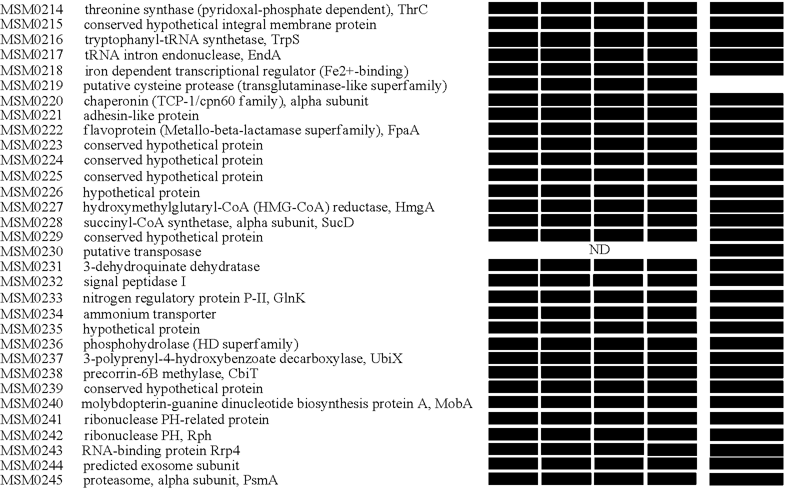

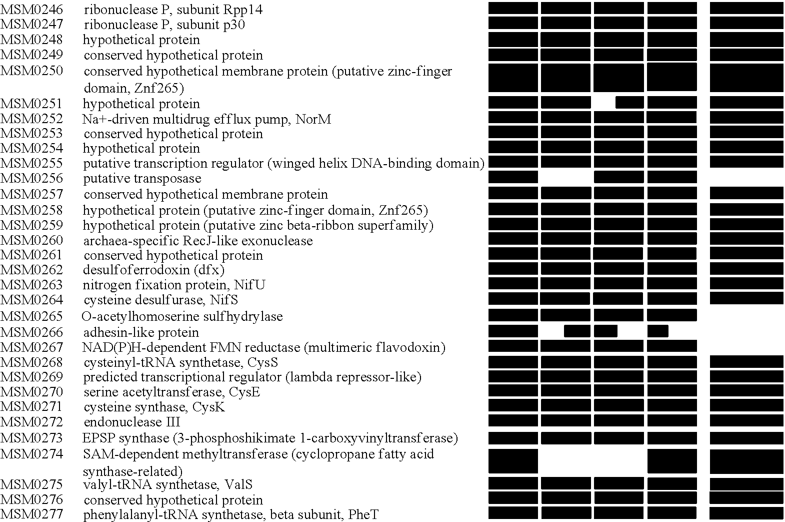

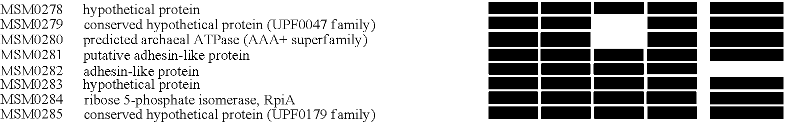

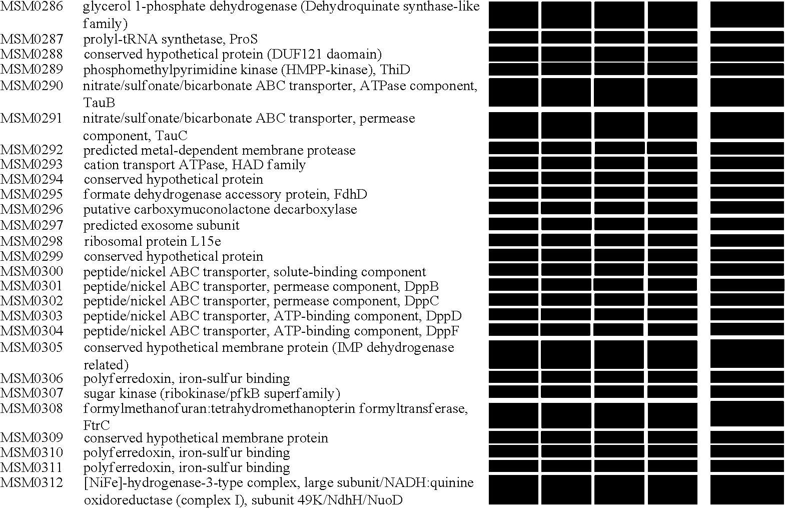

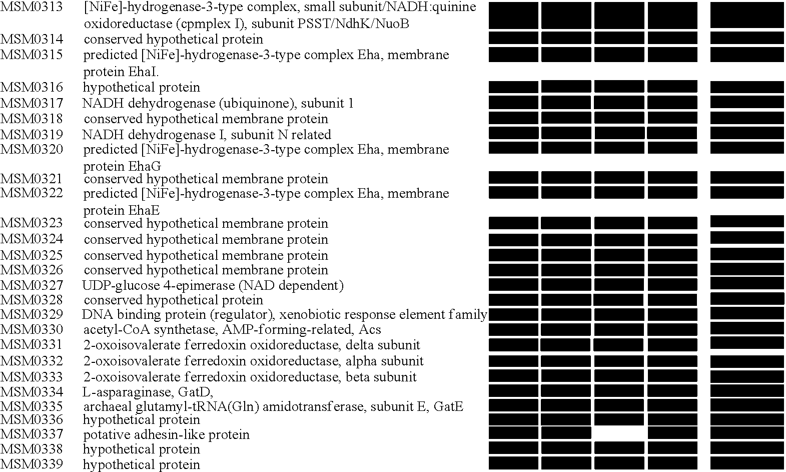

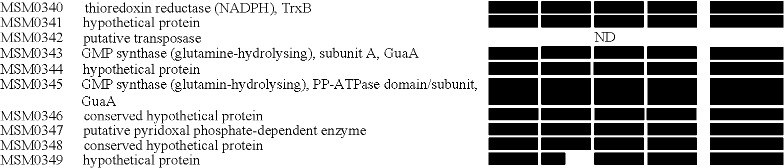

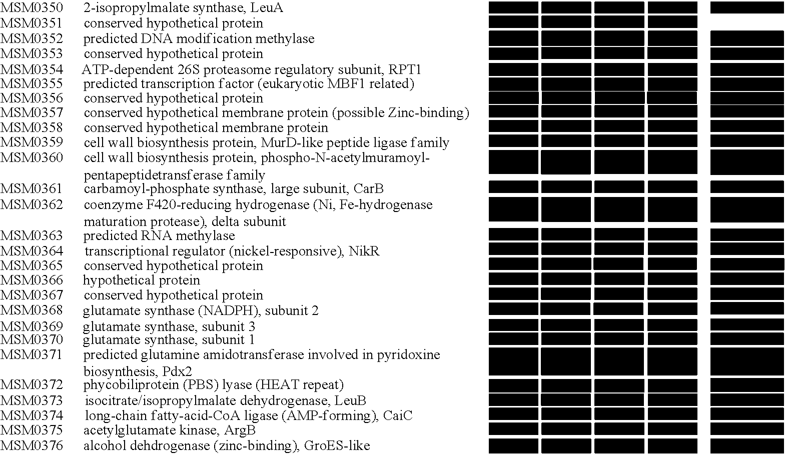

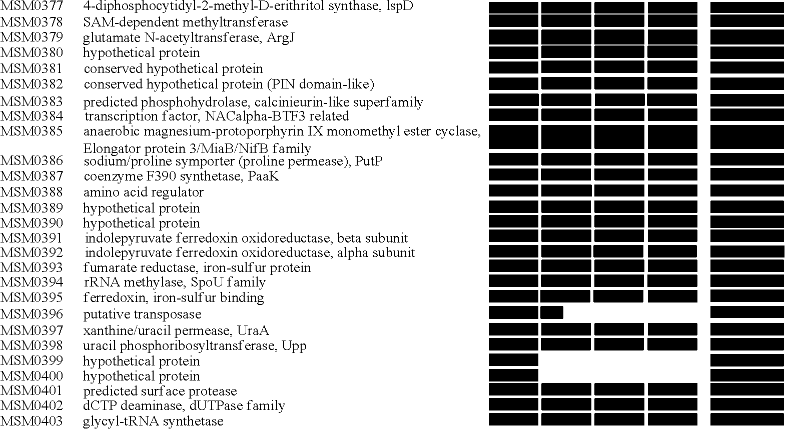

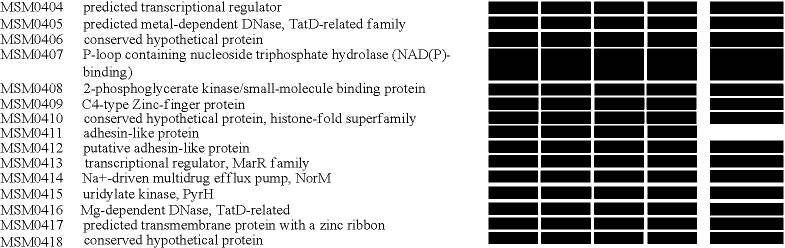

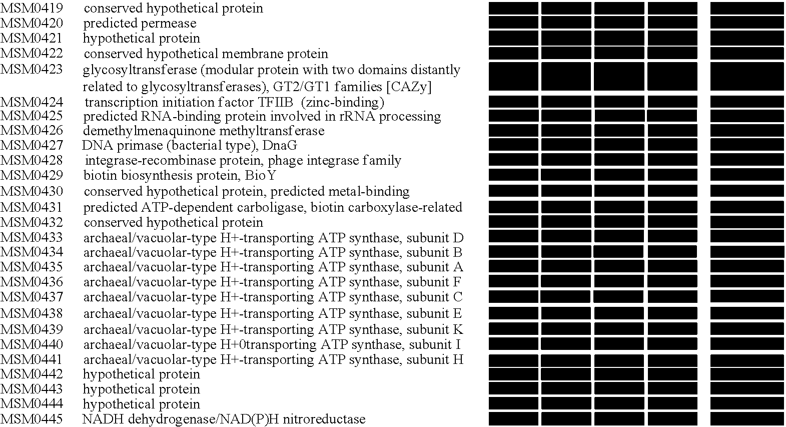

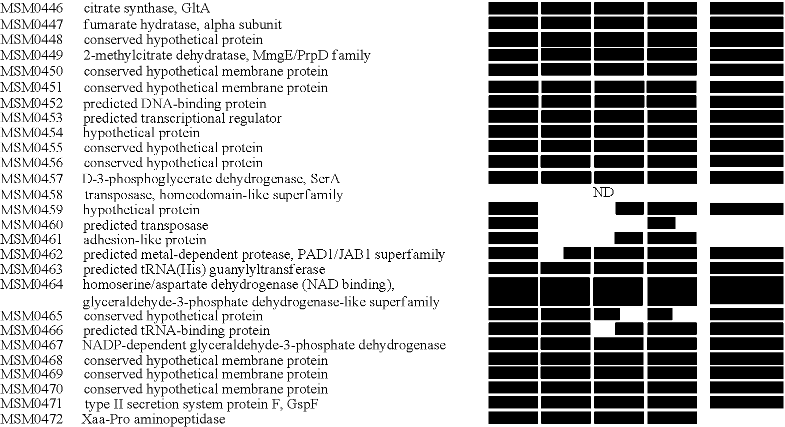

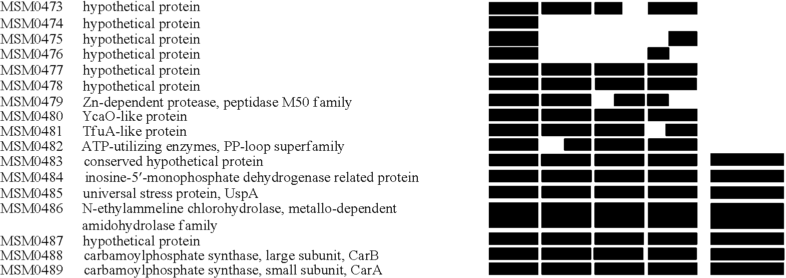

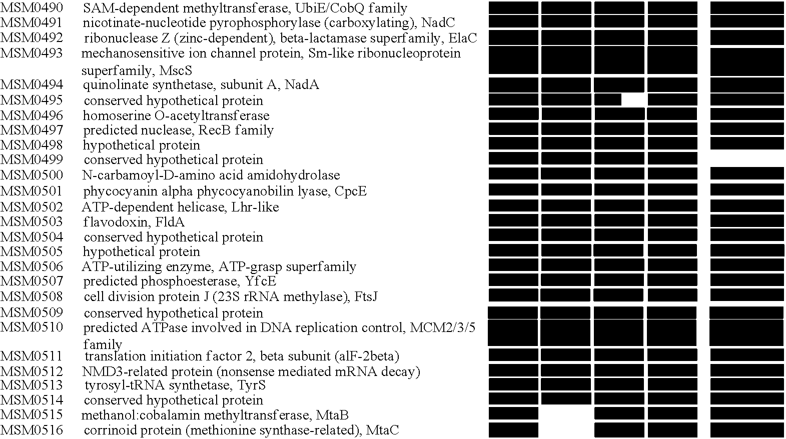

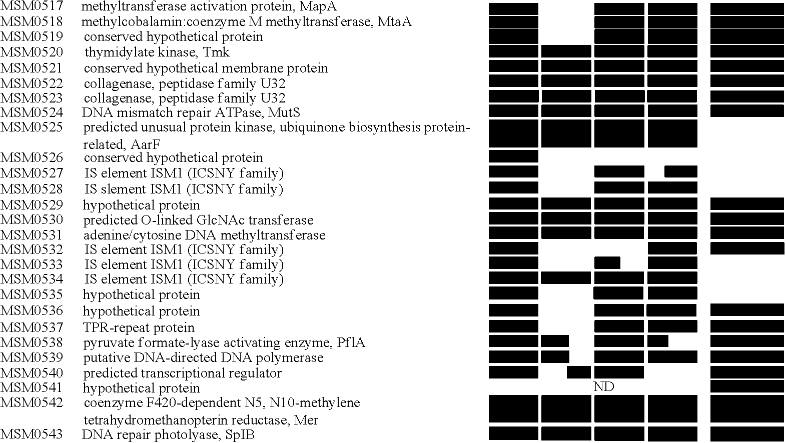

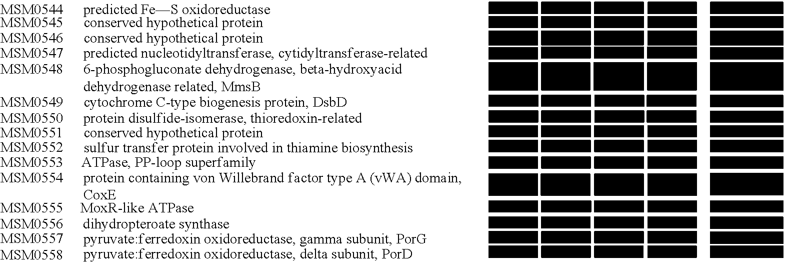

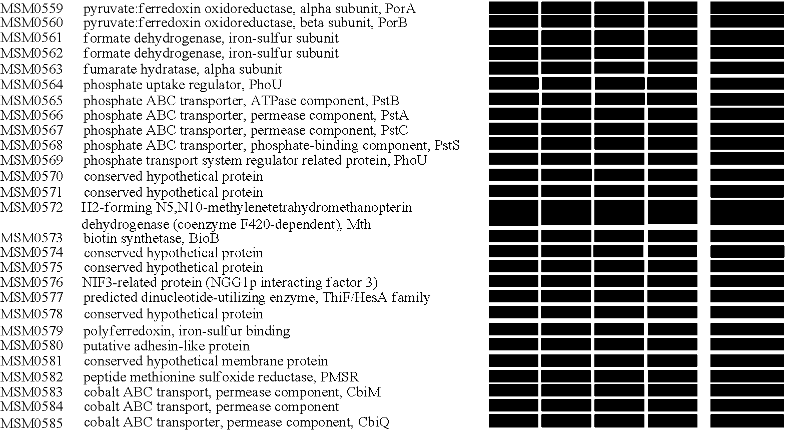

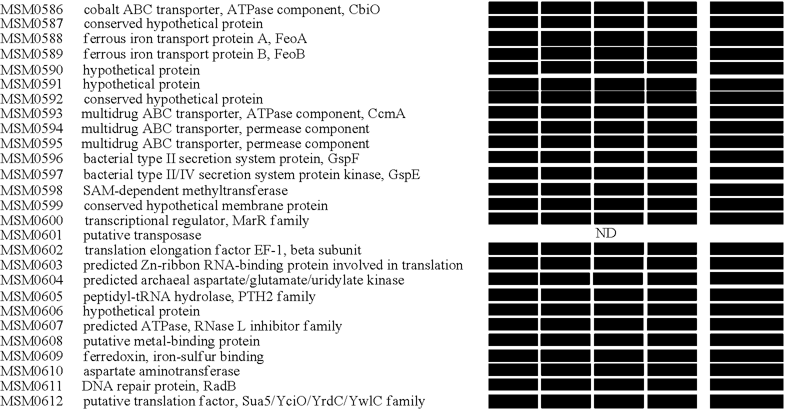

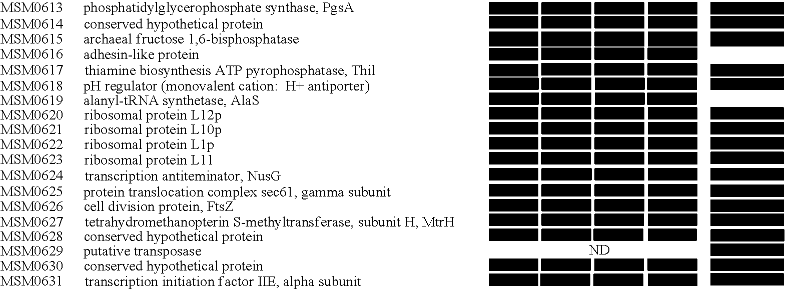

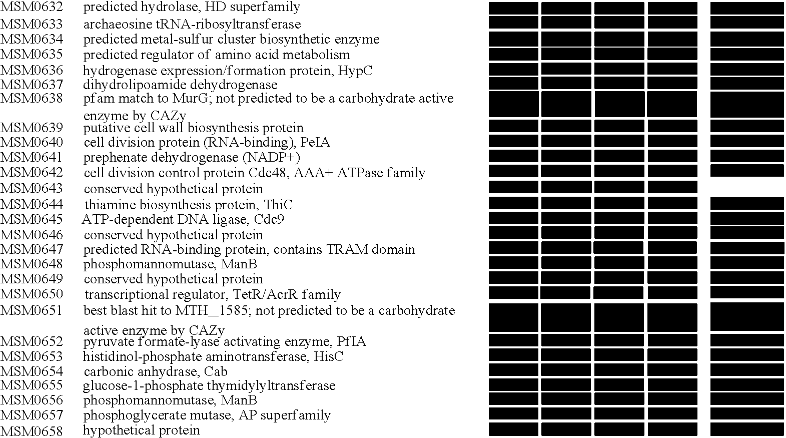

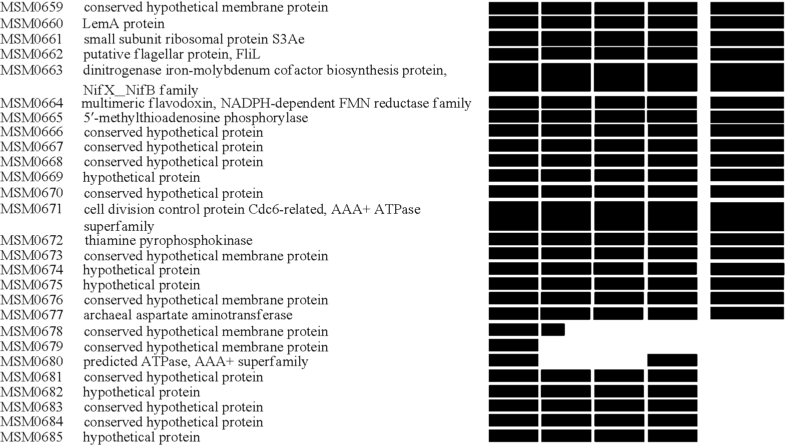

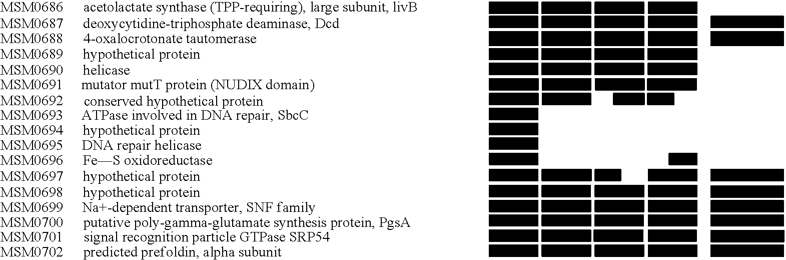

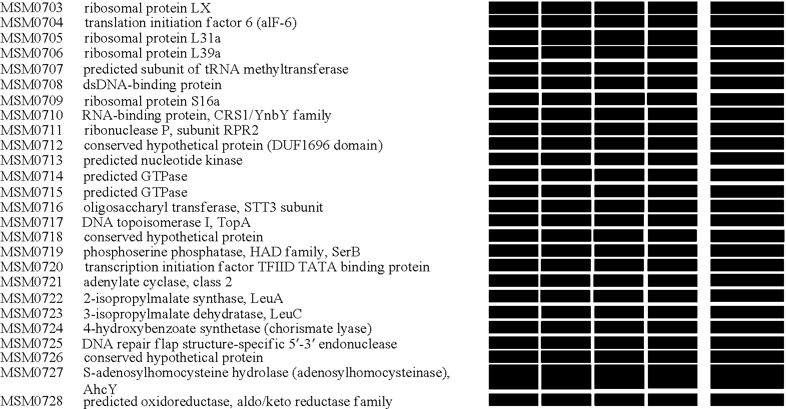

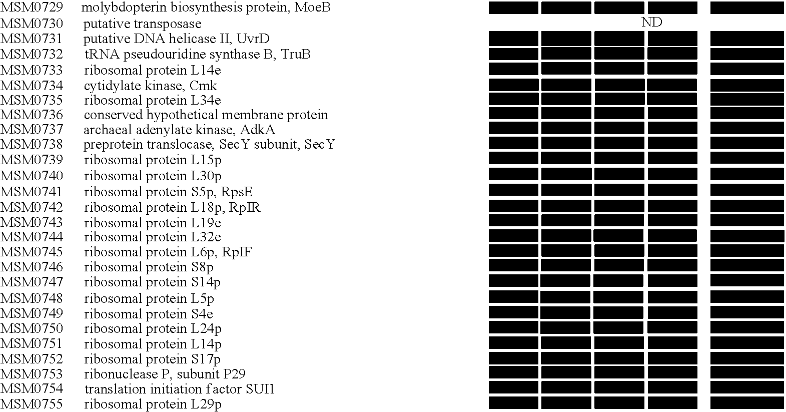

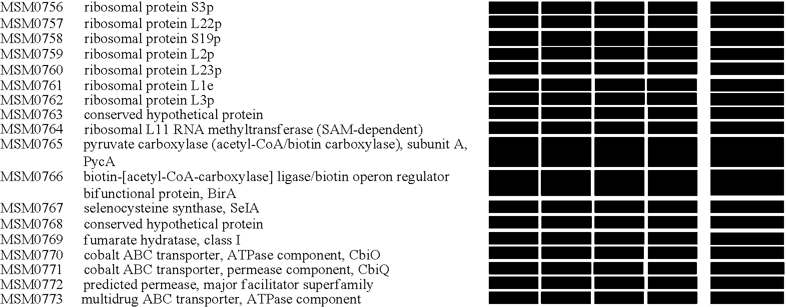

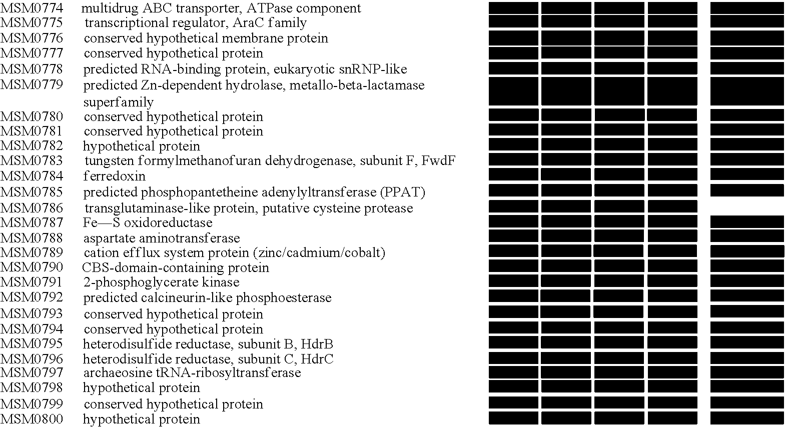

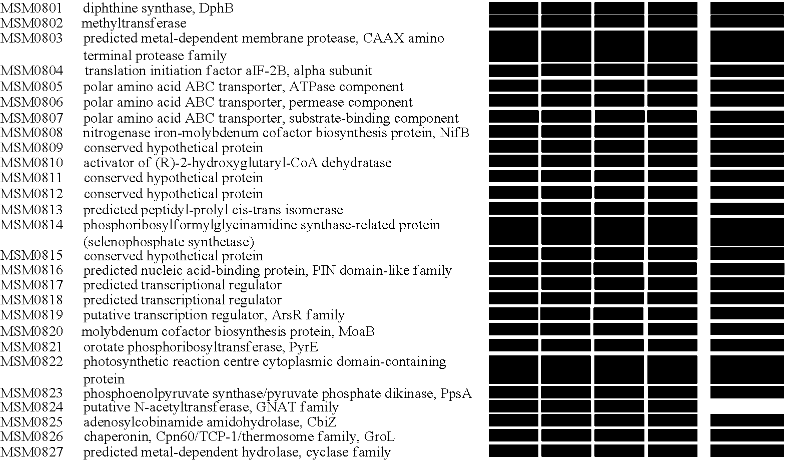

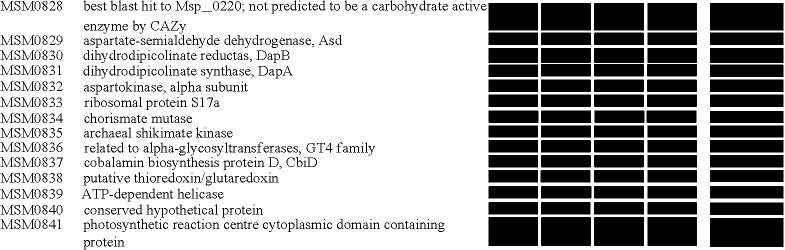

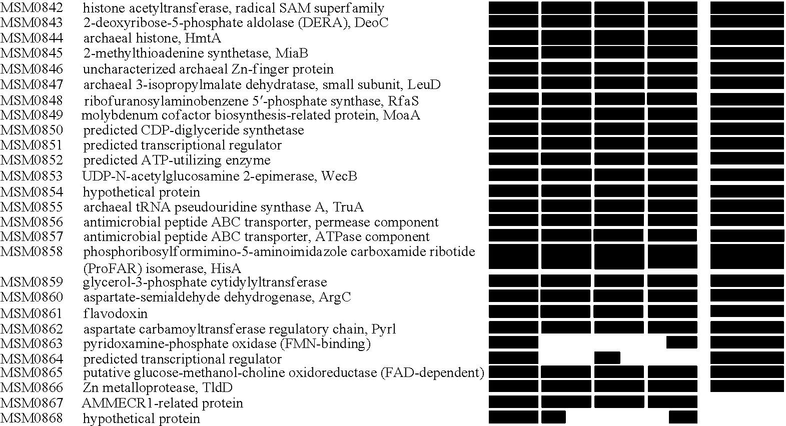

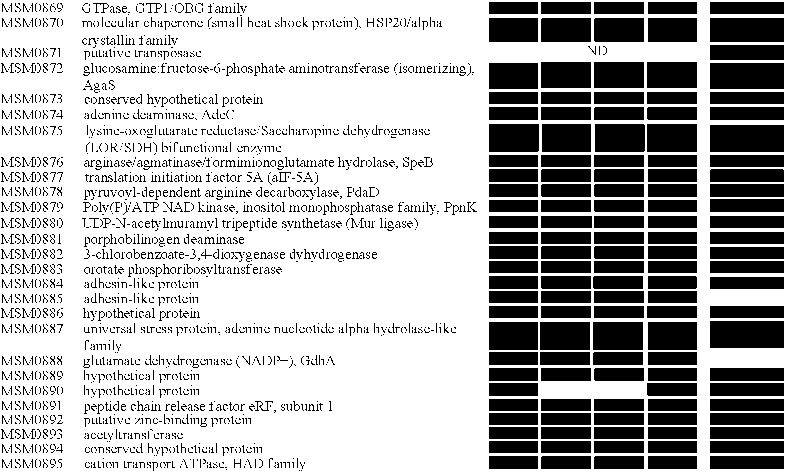

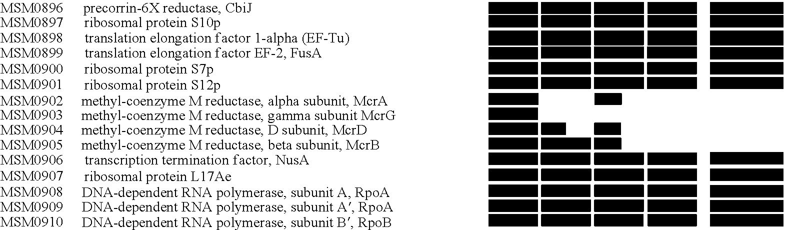

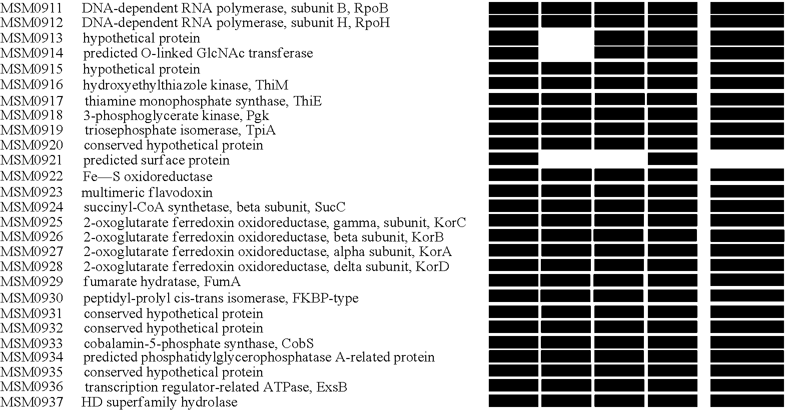

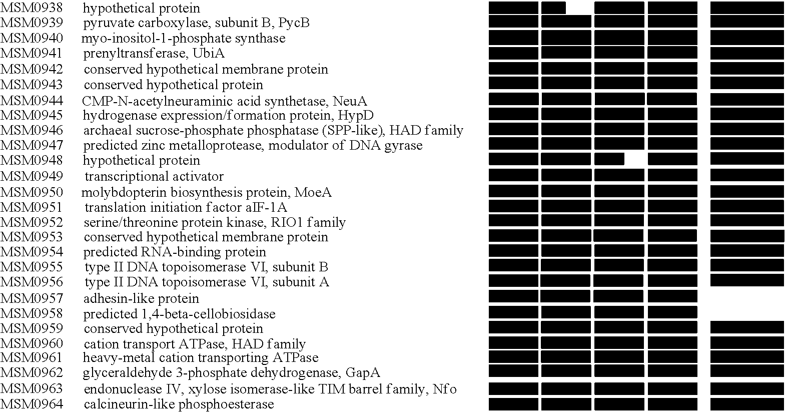

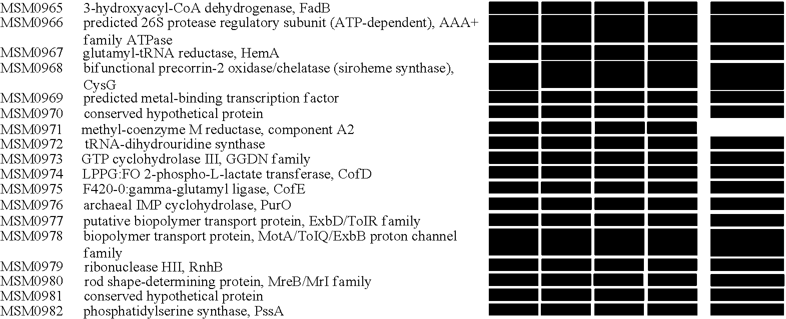

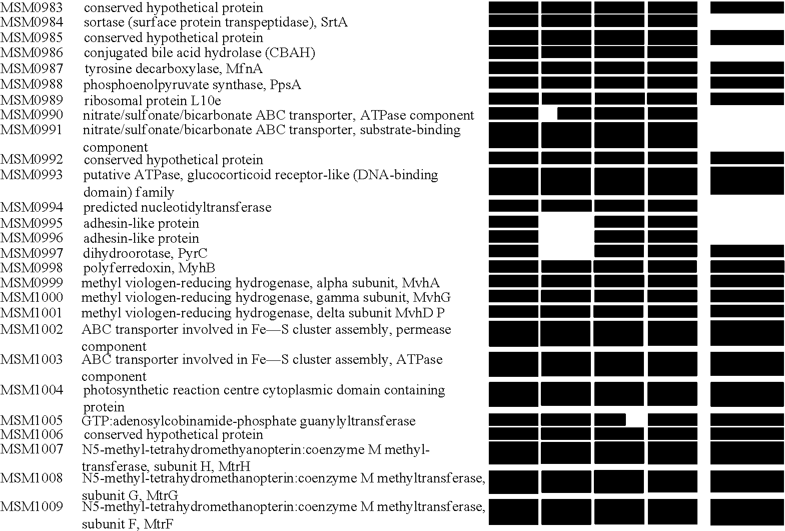

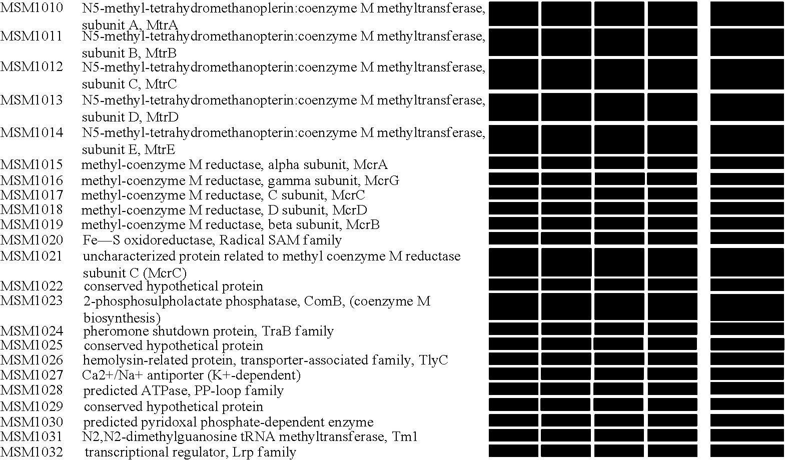

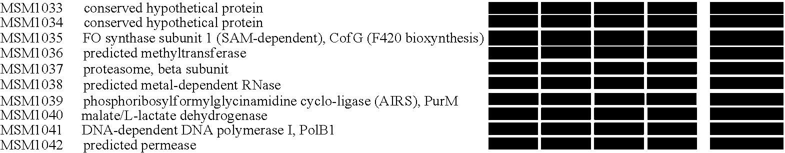

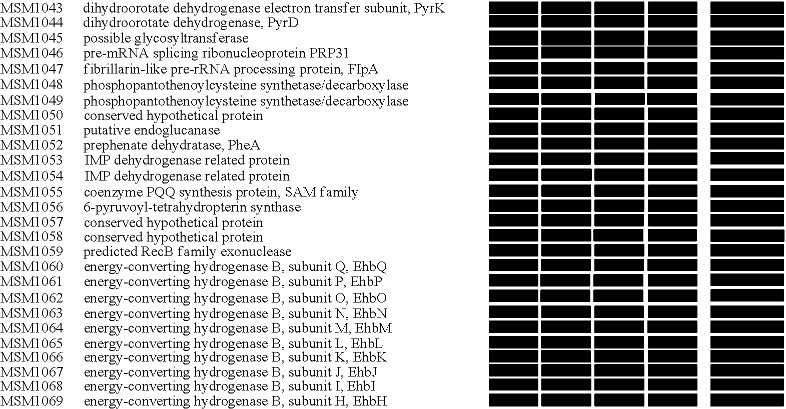

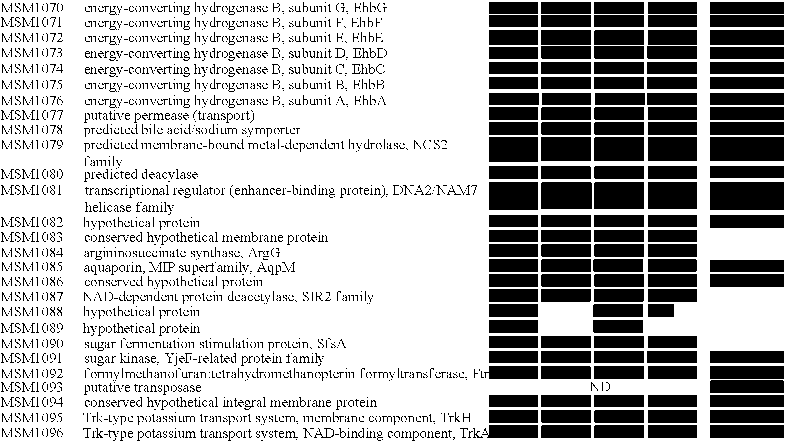

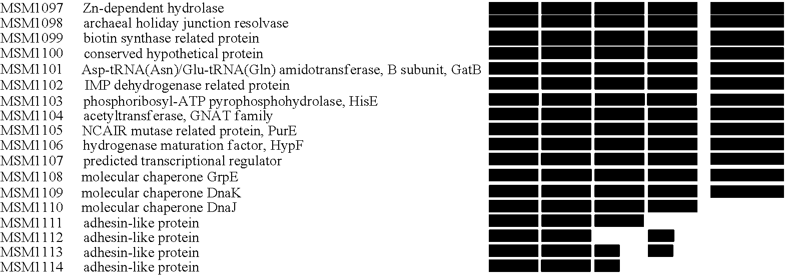

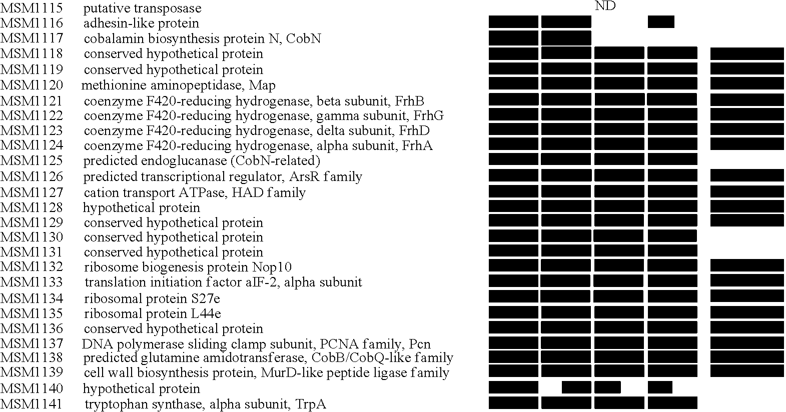

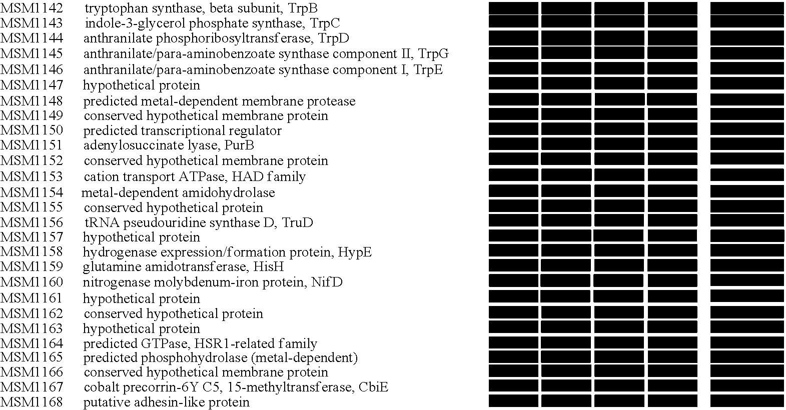

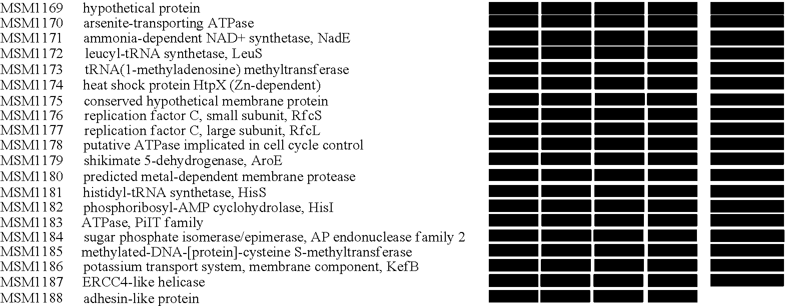

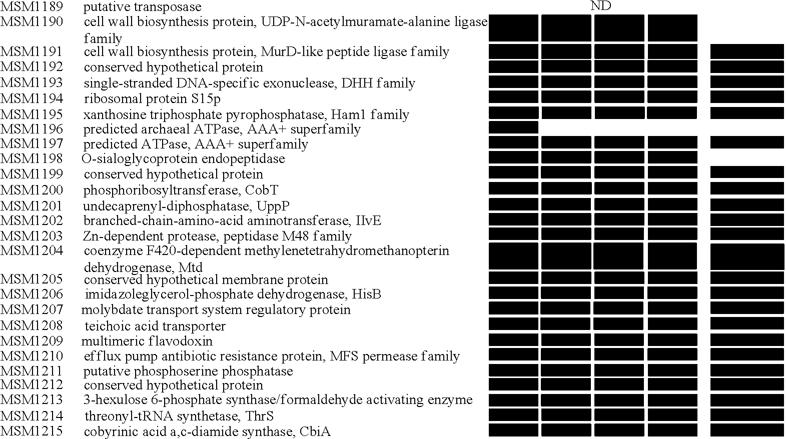

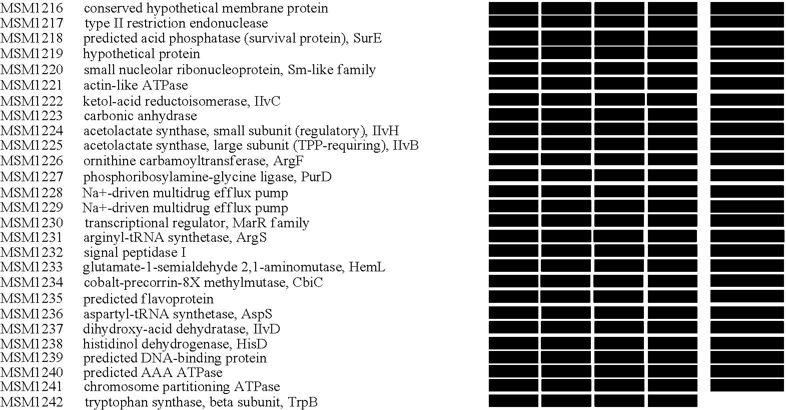

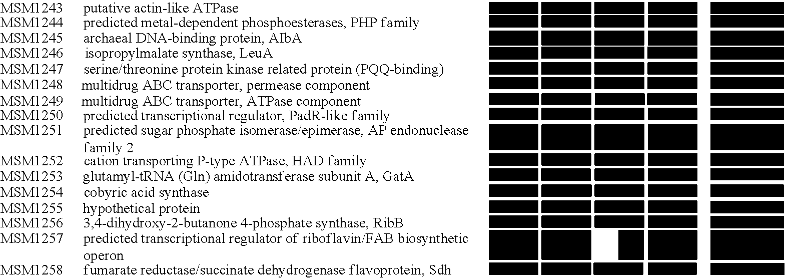

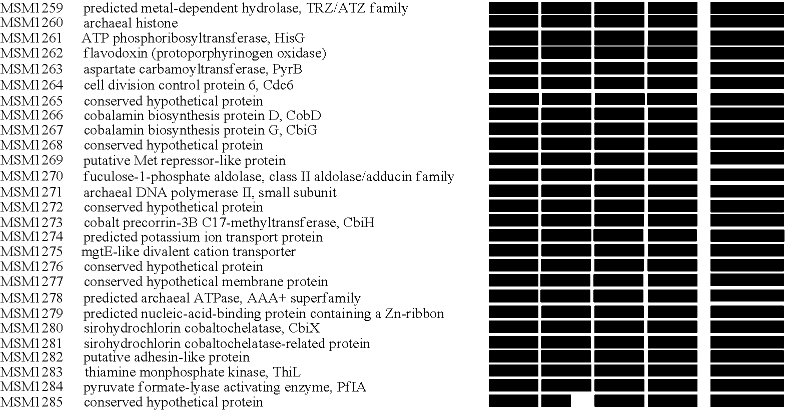

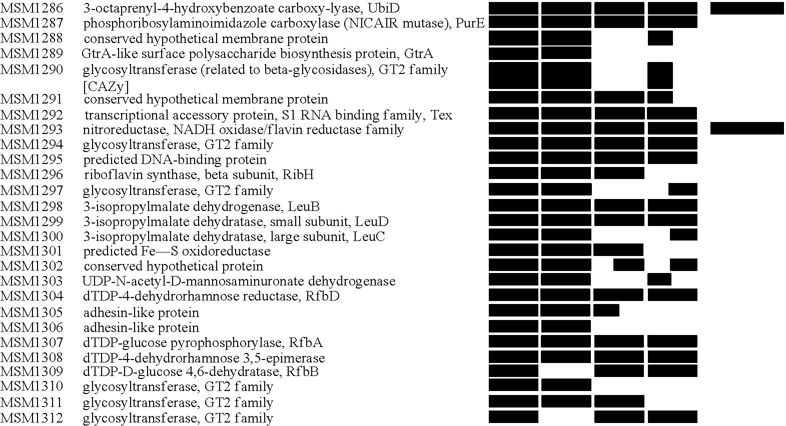

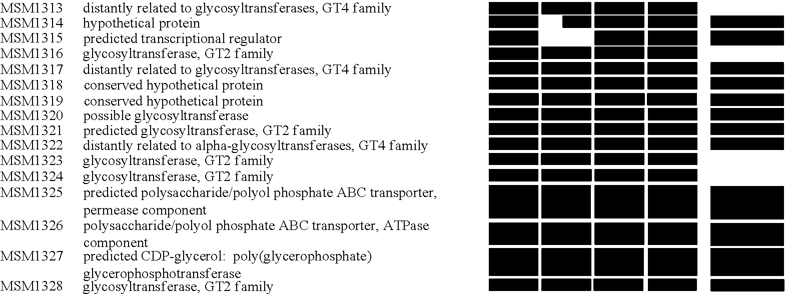

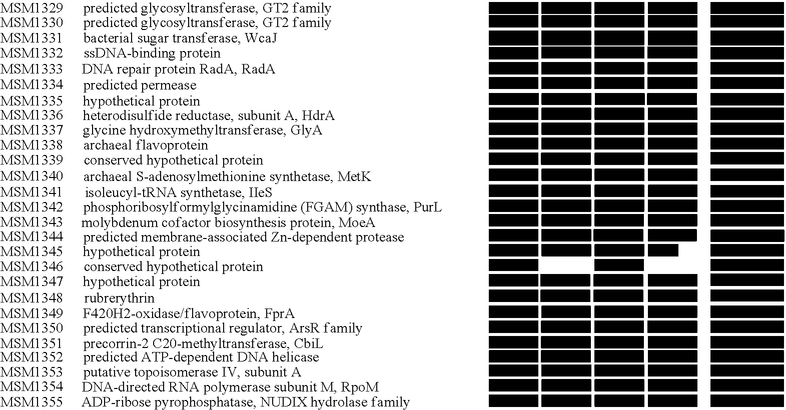

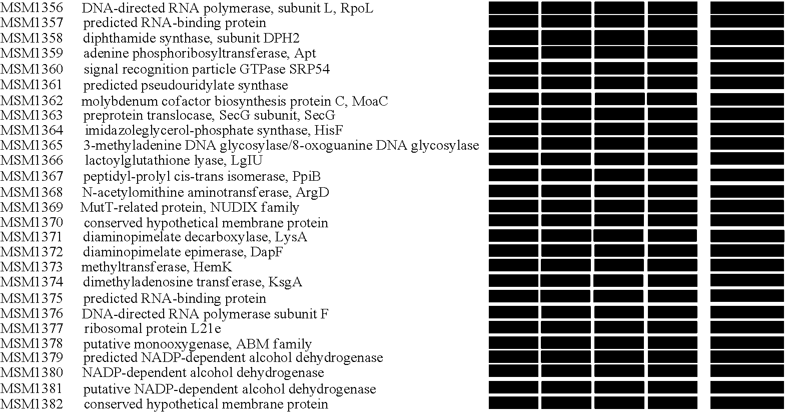

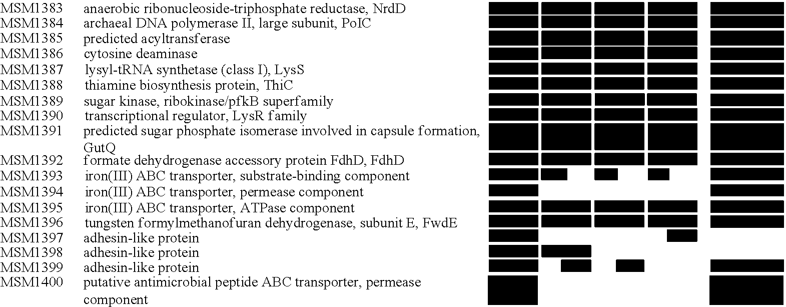

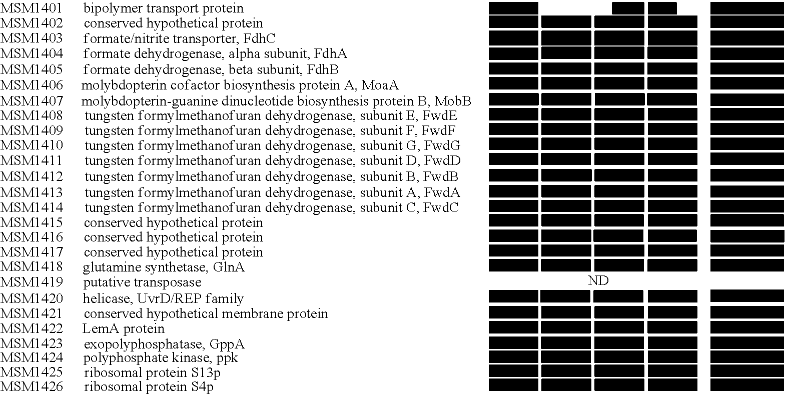

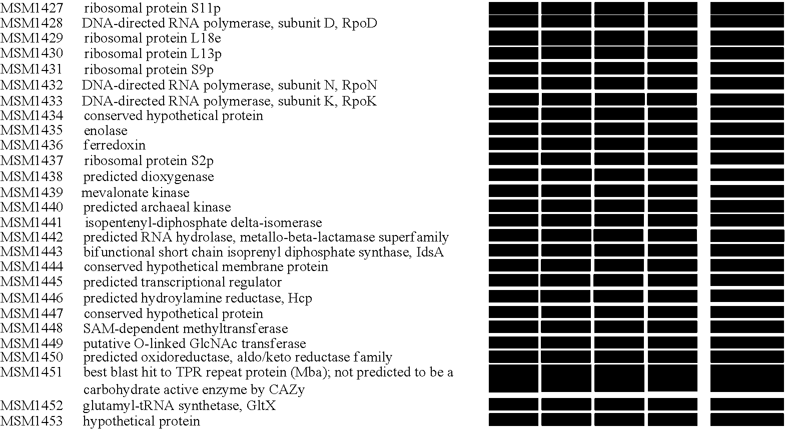

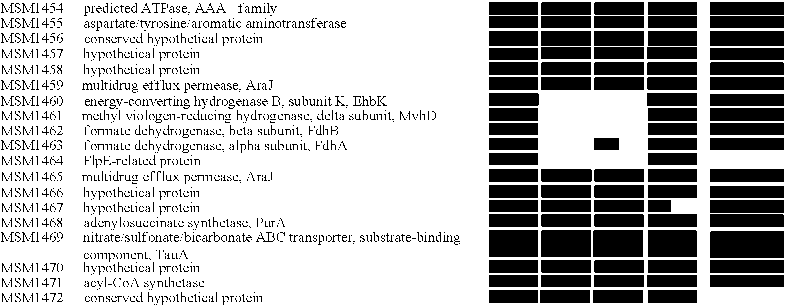

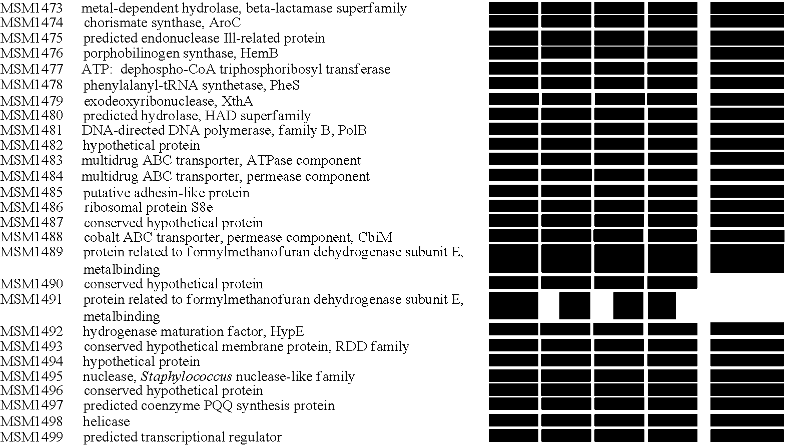

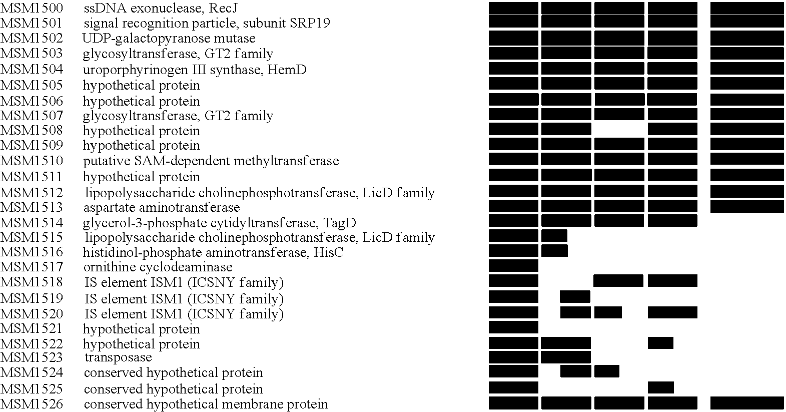

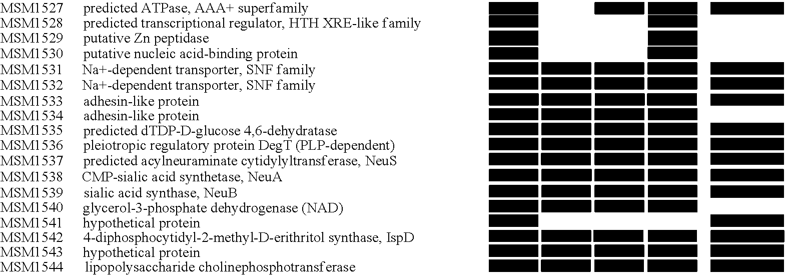

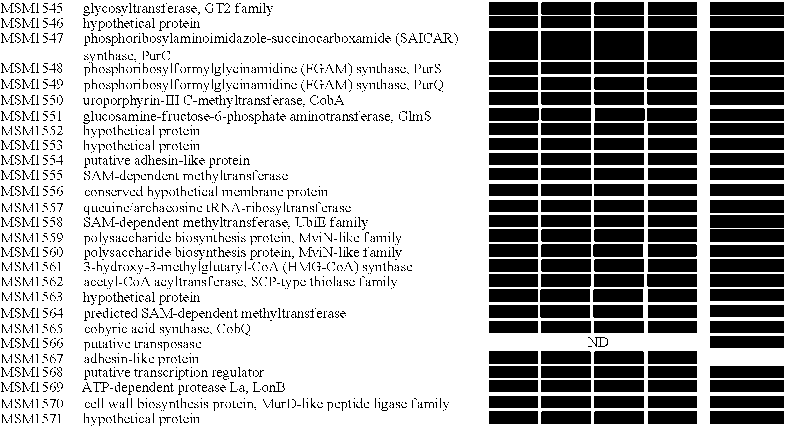

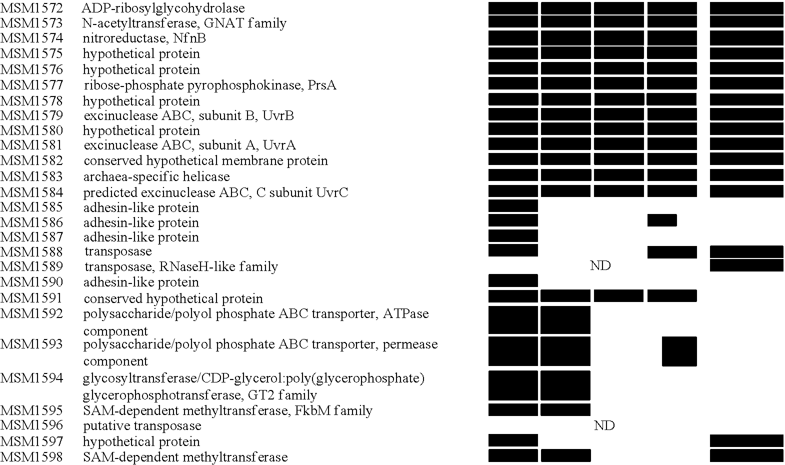

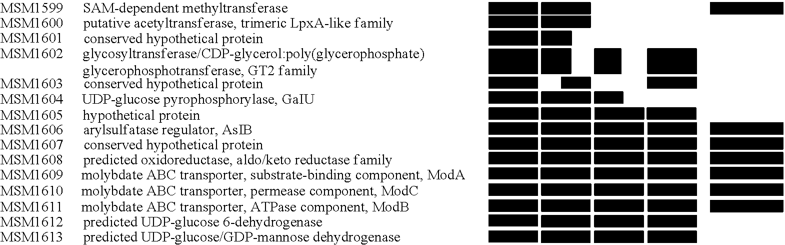

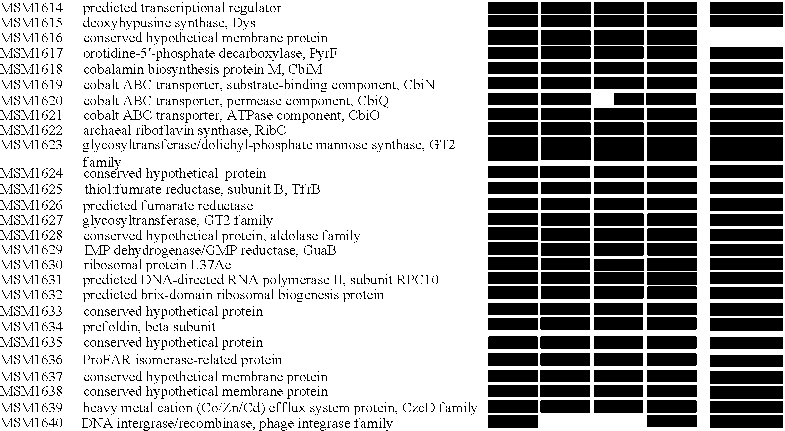

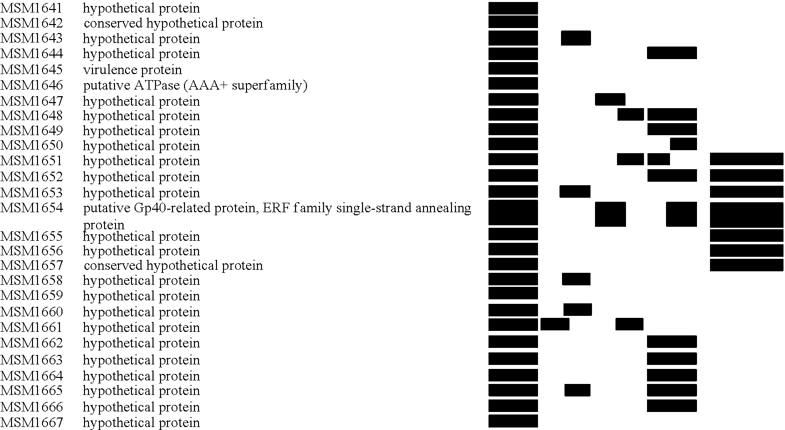

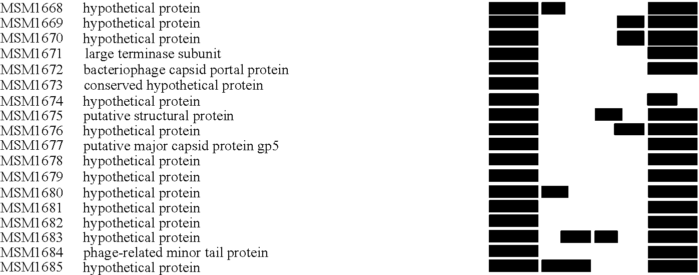

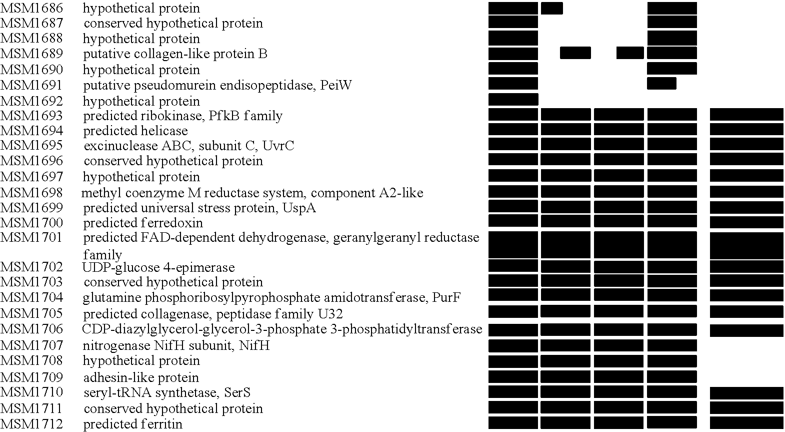

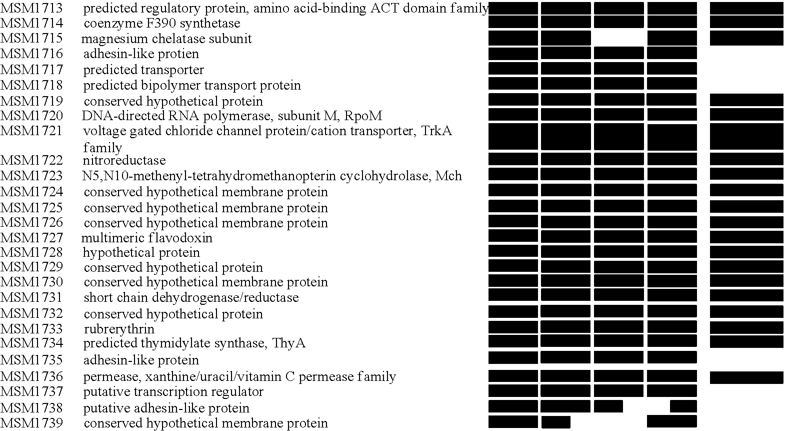

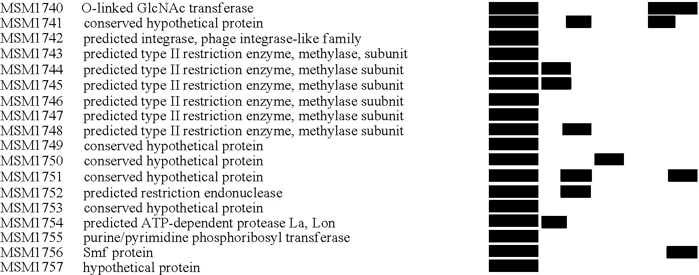

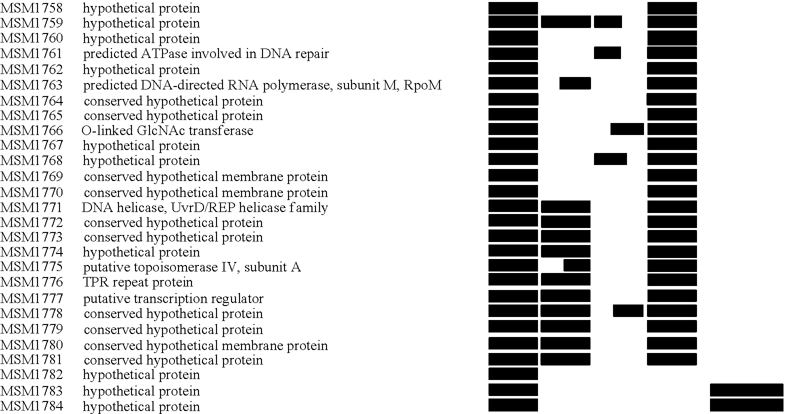

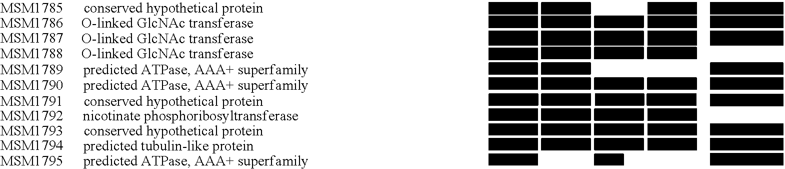

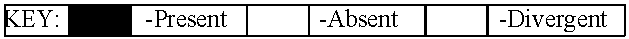

¹GeneChip-based genotyping of M. smithii strains done in duplicate; ‘present’ or ‘absent’ calls were determined using a perfect match/mismatch (PM/MM) model in dChip (see Methods). Note that the term ‘absent’ is based on different criteria than those used for the human microbiome dataset (see footnote 2).
²Metagenomic datasets from the microbiomes of two healthy lean adults (Gill et al., 2006) were tested for identity of M. smithii PS ORTs; ORFs with reads that matched with >95% identity are called ‘present,’ 80-95% identity are called ‘divergent’, and <80% identity are called ‘absent’.
ⁱⁱProbeset for M. smithii gene not represented on GeneChip.

TABLE 3
Transcriptional regulators identified in the M. smithii proteome
ORF COG ANNOTATION

| | | |
|---|---|---|
| MSM0026 | COG1396 | predicted transcriptional regulator (possible epoxidase activity) |
| MSM0094 | | predicted transcription regulator (TetR family) |
| MSM0155 | COG2061 | predicted allosteric regulator of homoserine dehydrogenase |
| MSM0218 | COG1321 | iron dependent transcriptional regulator (Fe2+-binding) |
| MSM0233 | COG0347 | nitrogen regulatory protein P-II, GlnK |
| MSM0255 | | putative transcription regulator (winged helix DNA-binding domain) |
| MSM0269 | COG2522 | predicted transcriptional regulator (lambda repressor-like) |
| MSM0329 | COG1396 | DNA binding protein, xenobiotic response element family |
| MSM0354 | COG1222 | ATP-dependent 26S proteasome regulatory subunit, RPT1 |
| MSM0364 | COG0864 | transcriptional regulator (nickel-responsive), NikR |
| MSM0383 | COG1409 | predicted phosphohydrolase, calcineurin-like superfamily |
| MSM0388 | COG4747 | amino acid regulator (ACT domain) |
| MSM0404 | COG4742 | predicted transcriptional regulator |
| MSM0413 | COG1846 | transcriptional regulator, MarR family |
| MSM0417 | COG4068 | predicted transmembrane protein with a zinc ribbon DNA-binding domain |
| MSM0452 | | predicted DNA-binding protein |
| MSM0453 | COG1395 | predicted transcriptional regulator |
| MSM0540 | COG2865 | predicted transcriptional regulator |
| MSM0564 | COG0704 | phosphate uptake regulator, PhoU |
| MSM0569 | COG0704 | phosphate transport system regulator related protein, PhoU |
| MSM0600 | COG1846 | transcriptional regulator, MarR family |
| MSM0635 | COG2150 | predicted regulator of amino acid metabolism |
| MSM0650 | COG1309 | transcriptional regulator, TetR/AcrR family |
| MSM0766 | COG0340 | biotin-[acetyl-CoA-carboxylase] ligase/biotin operon regulator bifunctional protein, BirA |
| MSM0775 | COG2207 | transcriptional regulator, AraC family |
| MSM0817 | COG4742 | predicted transcriptional regulator |
| MSM0818 | COG4742 | predicted transcriptional regulator |
| MSM0819 | COG0640 | putative transcription regulator, ArsR family (winged helix DNA-binding domain) |
| MSM0851 | COG1548 | predicted transcriptional regulator |
| MSM0862 | COG1781 | aspartate carbamoyltransferase regulatory chain, PyrI |
| MSM0864 | COG1733 | predicted transcriptional regulator |
| MSM0936 | COG0603 | transcription regulator-related ATPase, ExsB |
| MSM0966 | COG1223 | predicted 26S protease regulatory subunit (ATP-dependent), AAA+ family ATPase |
| MSM1030 | COG0399 | predicted pyridoxal phosphate-dependent enzyme |
| MSM1032 | COG1522 | transcriptional regulator, Lrp family |
| MSM1081 | COG1112 | transcriptional regulator, DNA2/NAM7 helicase family |
| MSM1090 | COG1489 | sugar fermentation stimulation protein, SfsA |
| MSM1106 | COG0068 | hydrogenase maturation factor, HypF |
| MSM1107 | COG1777 | predicted transcriptional regulator |
| MSM1126 | COG0640 | predicted transcriptional regulator, ArsR family (arsenic) |
| MSM1150 | COG1476 | predicted transcriptional regulator |
| MSM1207 | COG2005 | molybdate transport system regulatory protein |
| MSM1224 | COG0440 | acetolactate synthase, small subunit (regulatory), IlvH |
| MSM1230 | COG1846 | transcriptional regulator, MarR family |
| MSM1250 | COG1695 | predicted transcriptional regulator, PadR-like family |
| MSM1257 | COG1339 | predicted transcriptional regulator of riboflavin/FAD biosynthetic operon |
| MSM1292 | COG2183 | transcriptional accessory protein, S1 RNA binding family, Tex |
| MSM1315 | COG2865 | predicted transcriptional regulator |
| MSM1350 | COG0640 | predicted transcriptional regulator, ArsR family |
| MSM1390 | COG0583 | transcriptional regulator, LysR family |
| MSM1445 | COG1378 | predicted transcriptional regulator |
| MSM1499 | COG1497 | predicted transcriptional regulator |
| MSM1528 | COG1396 | predicted transcriptional regulator, HTH XRE-like family (xenobiotic) |
| MSM1536 | COG0399 | pleiotropic regulatory protein DegT (PLP-dependent) |
| MSM1568 | | putative transcription regulator |
| MSM1606 | COG0641 | arylsulfatase regulator, AslB |
| MSM1614 | COG2524 | predicted transcriptional regulator |
| MSM1713 | COG4747 | predicted regulatory protein, amino acid-binding ACT domain family |
| MSM1737 | | putative transcription regulator |
| MSM1777 | | putative transcription regulator |

TABLE 4
Machinery for genome evolution in M. smithii
ORF ANNOTATION

| | | |
|---|---|---|
| Restriction | MSM0157 | predicted type I restriction-modification enzyme, subunit S |
| Modification | MSM0158 | type I restriction-modification system methylase, subunit S |
| System | MSM1187 | predicted type III restriction enzyme |
| Subunits | MSM1217 | type II restriction endonuclease |
| | MSM1743 | predicted type II restriction enzyme, methylase subunit |
| | MSM1744 | predicted type II restriction enzyme, methylase subunit |
| | MSM1745 | predicted type II restriction enzyme, methylase subunit |
| | MSM1746 | predicted type II restriction enzyme, methylase subunit |
| | MSM1747 | predicted type II restriction enzyme, methylase subunit |
| | MSM1748 | predicted type II restriction enzyme, methylase subunit |
| | MSM1752 | predicted restriction endonuclease |
| Recombination/ | MSM0023 | uncharacterized protein predicted to be involved in DNA repair |
| Repair | MSM0097 | Mg-dependent DNase, TatD |
| | MSM0120 | purine NTPase involved in DNA repair, Rad50 |
| | MSM0121 | DNA repair exonuclease (SbcD/Mre11-family), Rad32 |
| | MSM0163 | conserved hypothetical proetin predicted to be involved in DNA repair |
| | MSM0164 | conserved hypothetical protein predicted to be involved in DNA repair |
| | MSM0167 | conserved hypothetical protein predicted to be involved in DNA repair |
| | MSM0168 | conserved hypothetical protein predicted to be involved in DNA repair |
| | MSM0170 | conserved hypothetical protein predicted to be involved in DNA repair |
| | MSM0405 | predicted metal-dependent DNase, TatD-related family |
| | MSM0416 | Mg-dependent DNase, TatD-related |
| | MSM0524 | DNA mismatch repair ATPase, MutS |
| | MSM0543 | DNA repair photolyase, SplB |
| | MSM0611 | DNA repair protein, RadB |
| | MSM0693 | ATPase involved in DNA repair, SbcC |
| | MSM0695 | DNA repair helicase |
| | MSM0725 | DNA repair flap structure-specific 5′-3′ endonuclease |
| | MSM1193 | single-stranded DNA-specific exonuclease, DHH family |
| | MSM1333 | DNA repair protein RadA, RadA |
| | MSM1500 | ssDNA exonuclease, RecJ |
| | MSM1640 | DNA intergrase/recombinase, phage integrase family |
| | MSM1761 | predicted ATPase involved in DNA repair |
| IS elements | MSM0527 | IS element ISM1 (ICSNY family) |
| | MSM0528 | IS element ISM1 (ICSNY family) |
| | MSM0532 | IS element ISM1 (ICSNY family) |
| | MSM0533 | IS element ISM1 (ICSNY family) |
| | MSM0534 | IS element ISM1 (ICSNY family) |
| | MSM1518 | IS element ISM1 (ICSNY family) |
| | MSM1519 | IS element ISM1 (ICSNY family) |
| | MSM1520 | IS element ISM1 (ICSNY family) |
| Transposases | MSM0008 | putative transposase |
| or | | |
| remnants of | MSM0087 | putative transposase |
| transposases | MSM0110 | predicted transposase |
| | MSM0230 | putative transposase |
| | MSM0256 | putative transposase |
| | MSM0342 | putative transposase |
| | MSM0396 | putative transposase |
| | MSM0458 | transposase, homeodomain-like superfamily |
| | MSM0460 | predicted transposase |
| | MSM0601 | putative transposase |
| | MSM0629 | putative transposase |
| | MSM0730 | putative transposase |
| | MSM0871 | putative transposase |
| | MSM1093 | putative transposase |
| | MSM1115 | putative transposase |
| | MSM1189 | putative transposase |
| | MSM1419 | putative transposase |
| | MSM1523 | transposase |
| | MSM1566 | putative transposase |
| | MSM1588 | predicted transposase |
| | MSM1589 | predicted transposase, RNaseH-like family |
| | MSM1596 | putative transposase |

TABLE 5
Publicly available finished genome sequences for members of Archaea

| | | | | | GenBank |
|---|---|---|---|---|---|
| | | | | Habitat of | Accession |
| Group | Strain Designation | Abbr. | Temp. | Origin | Number |
| Human Gut | Methanobrevibacter smithii PS (ATCC 35021) | Msm | Mesophilic | Host-associated | CP000678 |
| Methanogens | Methanosphaera stadtmanae DSM 3091 | Msp | Mesophilic | Host-associated | CP000102 |
| Non-Gut | Methanothermobacter thermautotrophicus | Mth | Thermophilic | Specialized | AE000666 |
| | Delta H | | | | |
| Methanogens | Methanocaldococcus jannaschii DSM 2661 | Mja | Hyperthermophilic | Aquatic | L77117 |
| | Methanococcoides burtonii DSM 6242 | Mbu | Mesophilic | Aquatic | CP000300 |
| | Methanococcus maripaludis S2 | Mmr | Mesophilic | Aquatic | BX950229 |
| | Methanopyrus kandleri AV19 | Mka | Hyperthermophilic | Specialized | AE009439 |
| | Methanosarcina acetivorans C2A | Mac | Mesophilic | Aquatic | AE010299 |
| | Methanosarcina barkeri str. Fusaro | Mba | Mesophilic | Multiple | CP000099 |
| | Methanosarcina mazei Go1 | Mma | Mesophilic | Multiple | AE008384 |
| | Methanospirillum hungatei JF-1 | Mhu | Mesophilic | Multiple | CP000254 |
| Other Archaea | Aeropyrum pernix K1 | Apx | Hyperthermophilic | Specialized | BA000002 |
| | Archaeoglobus fulgidus DSM 4304 | Afu | Hyperthermophilic | Aquatic | AE000782 |
| | Haloarcula marismortui ATCC 43049 | Hma | Mesophilic | Aquatic | AY596297 |
| | Halobacterium sp. NRC-1 | Hal | Mesophilic | Specialized | AE004437 |
| | Nanoarchaeum equitans Kin4-M | Neq | Hyperthermophilic | Host-associated | AE017199 |
| | Natronomonas pharaonis DSM 2160 | Nph | Mesophilic | Aquatic | CR936257 |
| | Picrophilus torridus DSM 9790 | Pto | Thermophilic | Specialized | AE017261 |
| | Pyrobaculum aerophilum str. IM2 | Pae | Hyperthermophilic | Aquatic | AE009441 |
| | Pyrococcus abyssi GE5 | Pab | Hyperthermophilic | Aquatic | AL096836 |
| | Pyrococcus furiosus DSM 3638 | Pfu | Hyperthermophilic | Aquatic | AE009950 |
| | Pyrococcus horikoshii OT3 | Pho | Hyperthermophilic | Aquatic | BA000001 |
| | Sulfolobus acidocaldarius DSM 639 | Sac | Thermophilic | Specialized | CP000077 |
| | Sulfolobus solfataricus P2 | Sso | Hyperthermophilic | Specialized | AE006641 |
| | Sulfolobus tokodaii str. 7 | Sto | Hyperthermophilic | Specialized | BA000023 |
| | Thermococcus kodakarensis KOD1 | Tko | Hyperthermophilic | Specialized | AP006878 |
| | Thermoplasma acidophilum DSM 1728 | Tac | Thermophilic | Specialized | AL139299 |
| | Thermoplasma volcanium GSS1 | Tvo | Thermophilic | Specialized | BA000011 |

TABLE 6
Representation of enriched gene ontology (GO) categories in the
M. smithii and M. stadtmanae proteomes compared to the
proteomes of all sequenced methanogenic archaea and all archaea

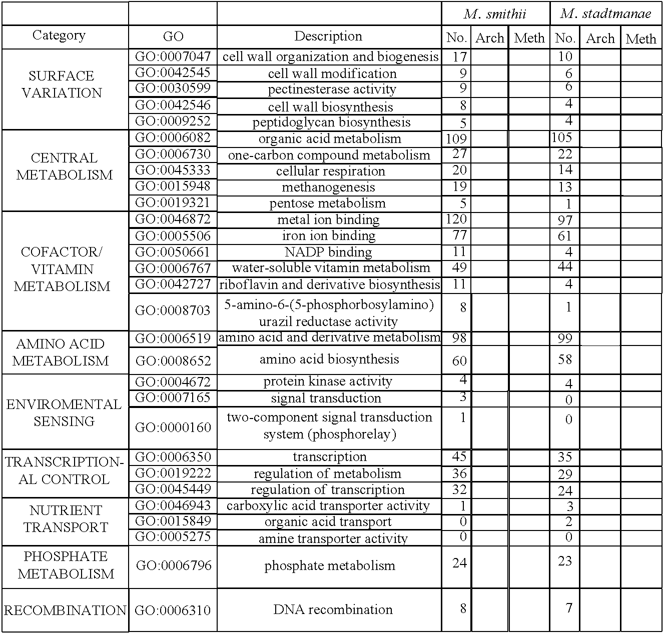

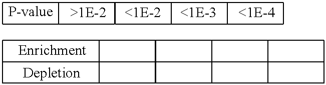

Abbreviations: ‘non-gut-associated methanogens’ (Meth) or ‘all Archaea’ (Arch) [see SI Table 5]; No., number of genes associated with gene ontology (GO)

TABLE 7
M. smithii genes in the significantly enriched GO categories listed in Table 6

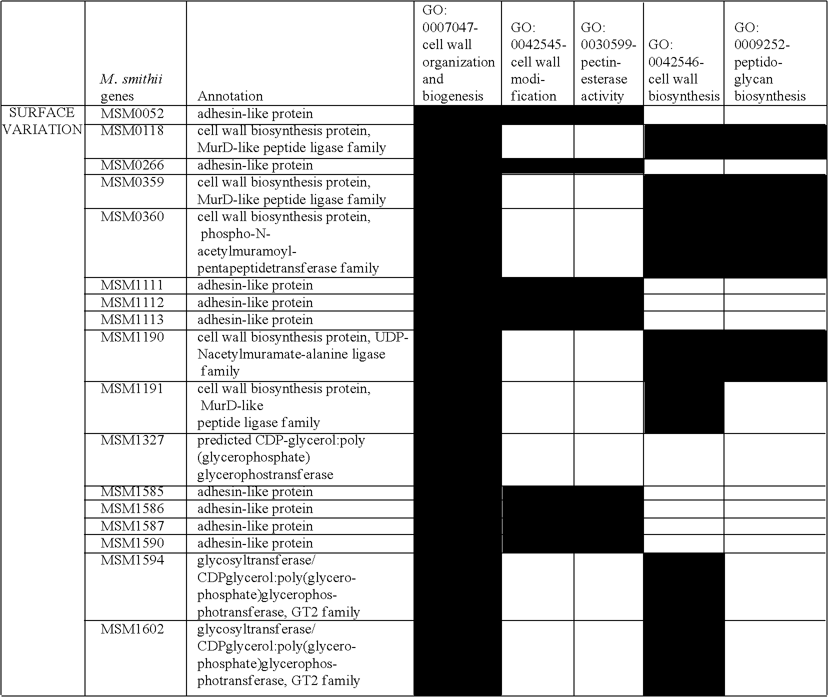

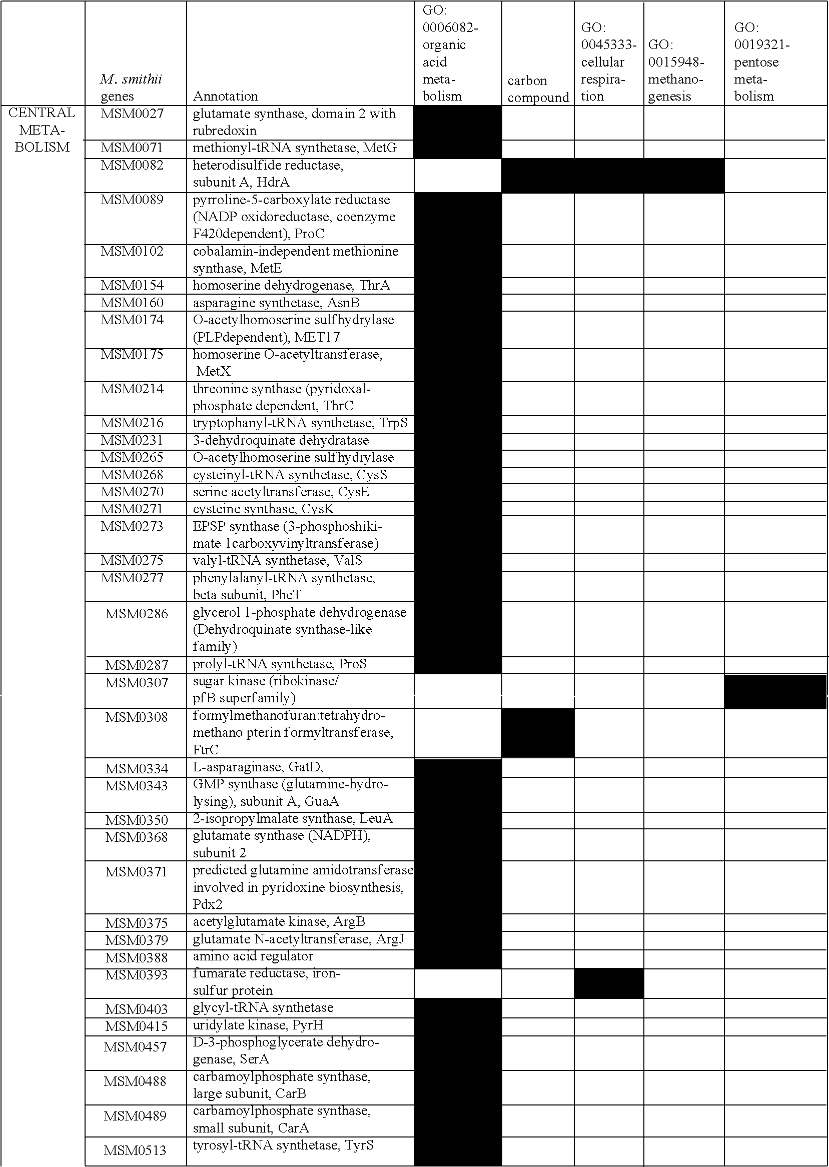

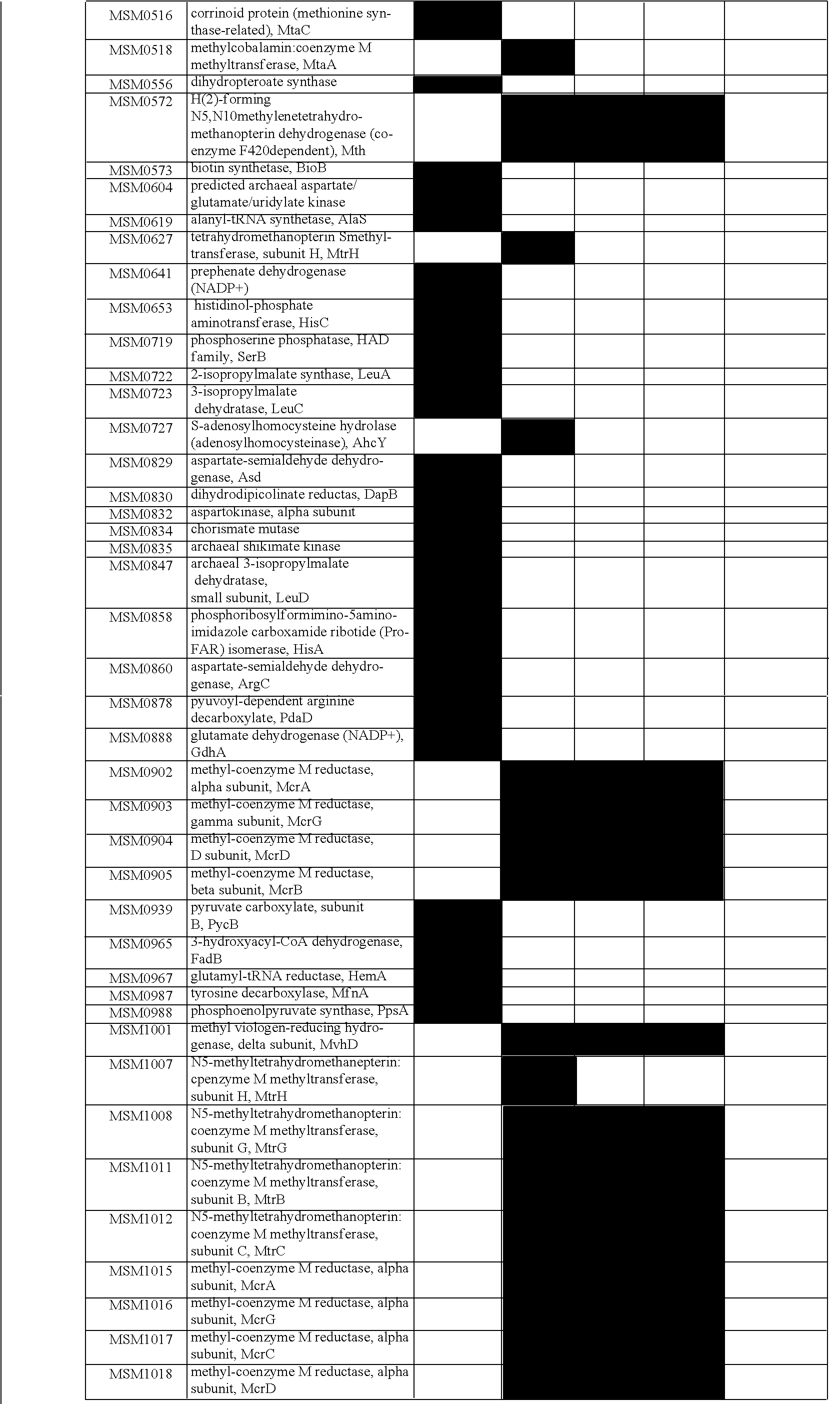

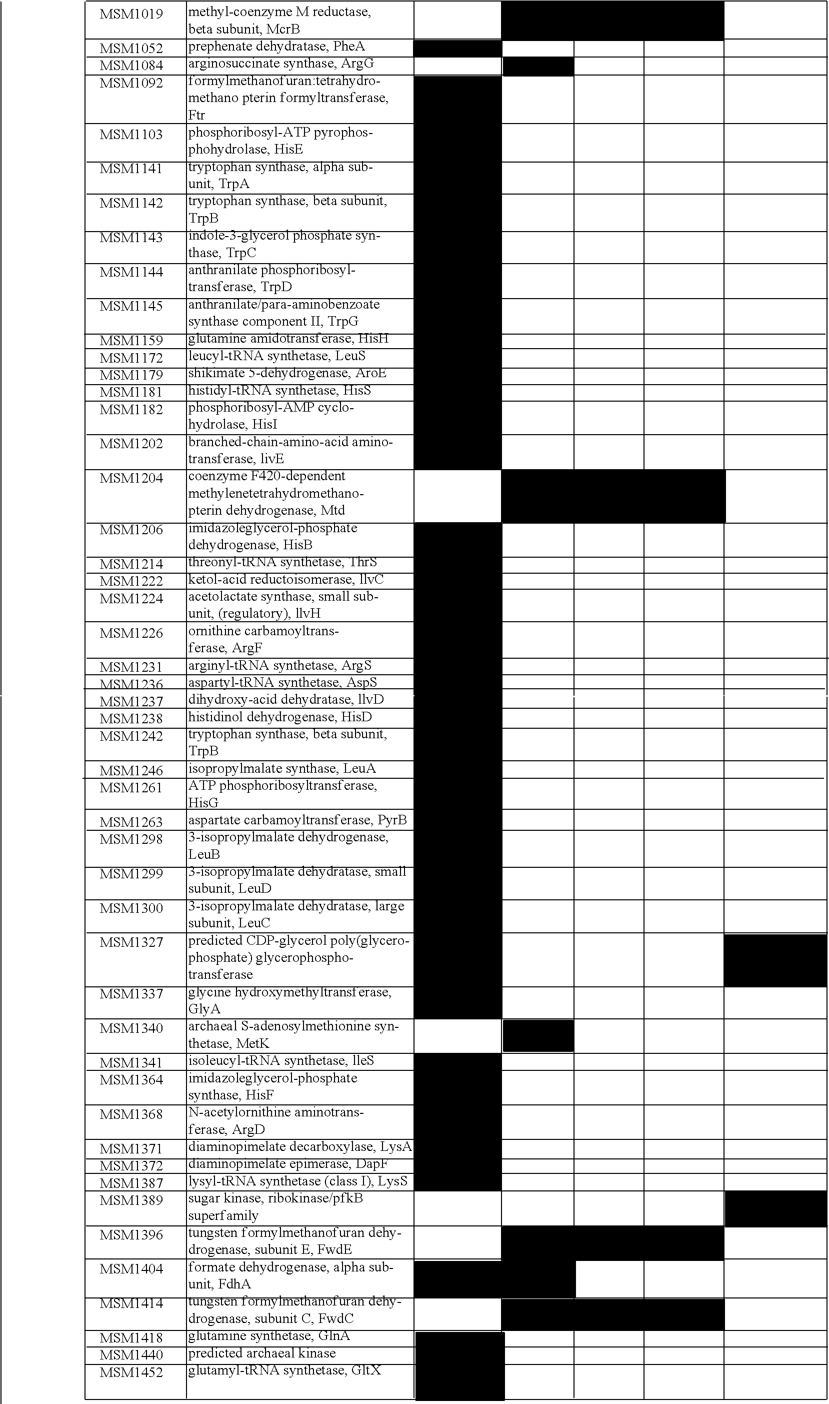

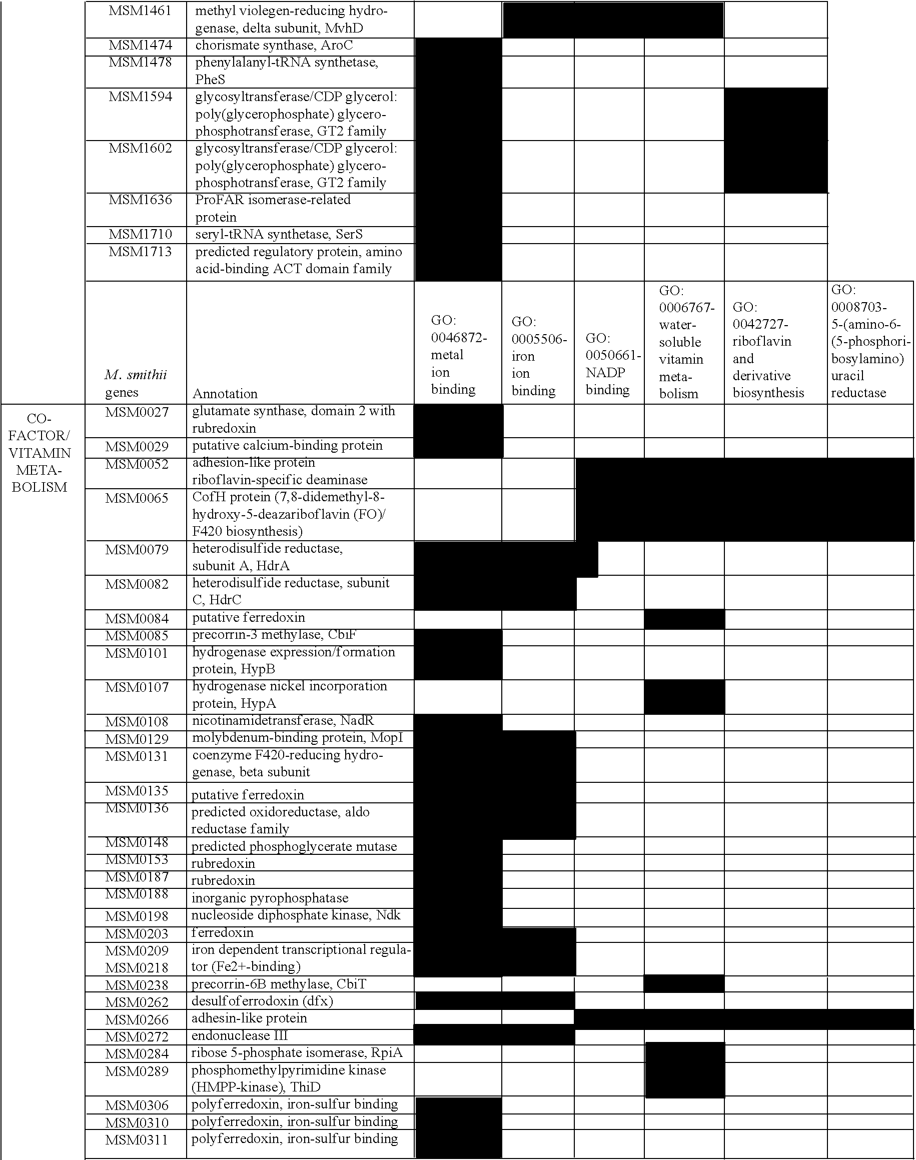

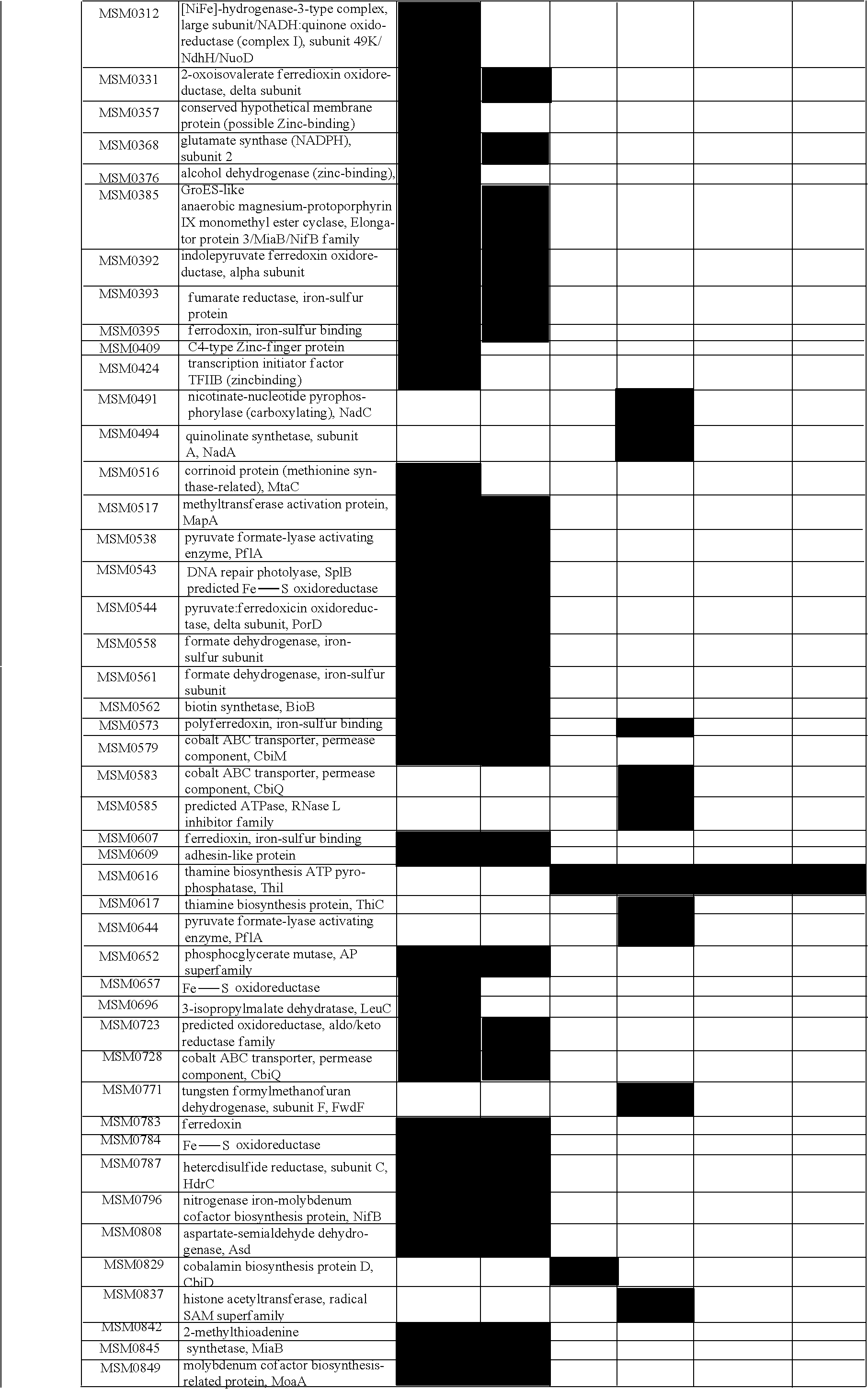

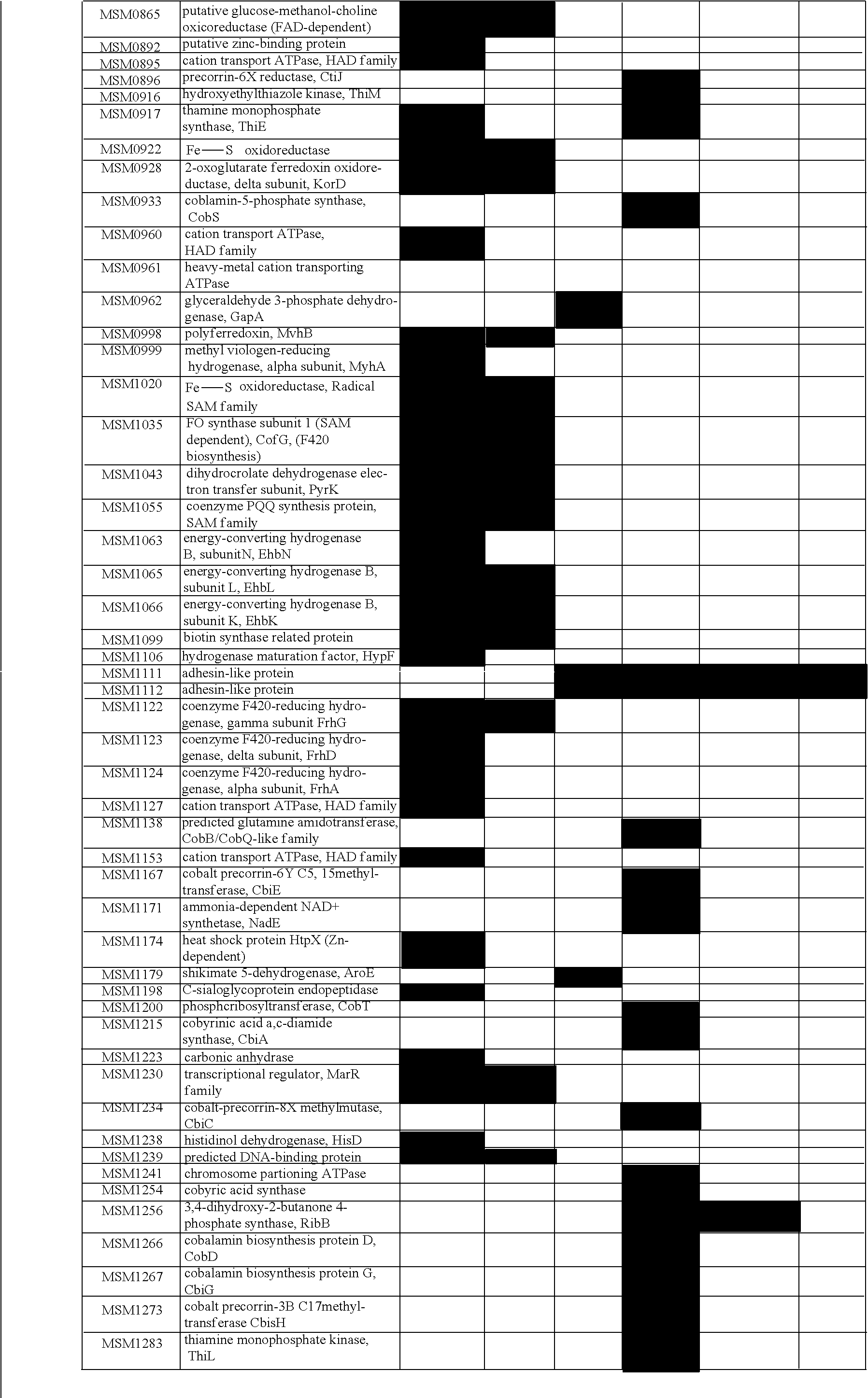

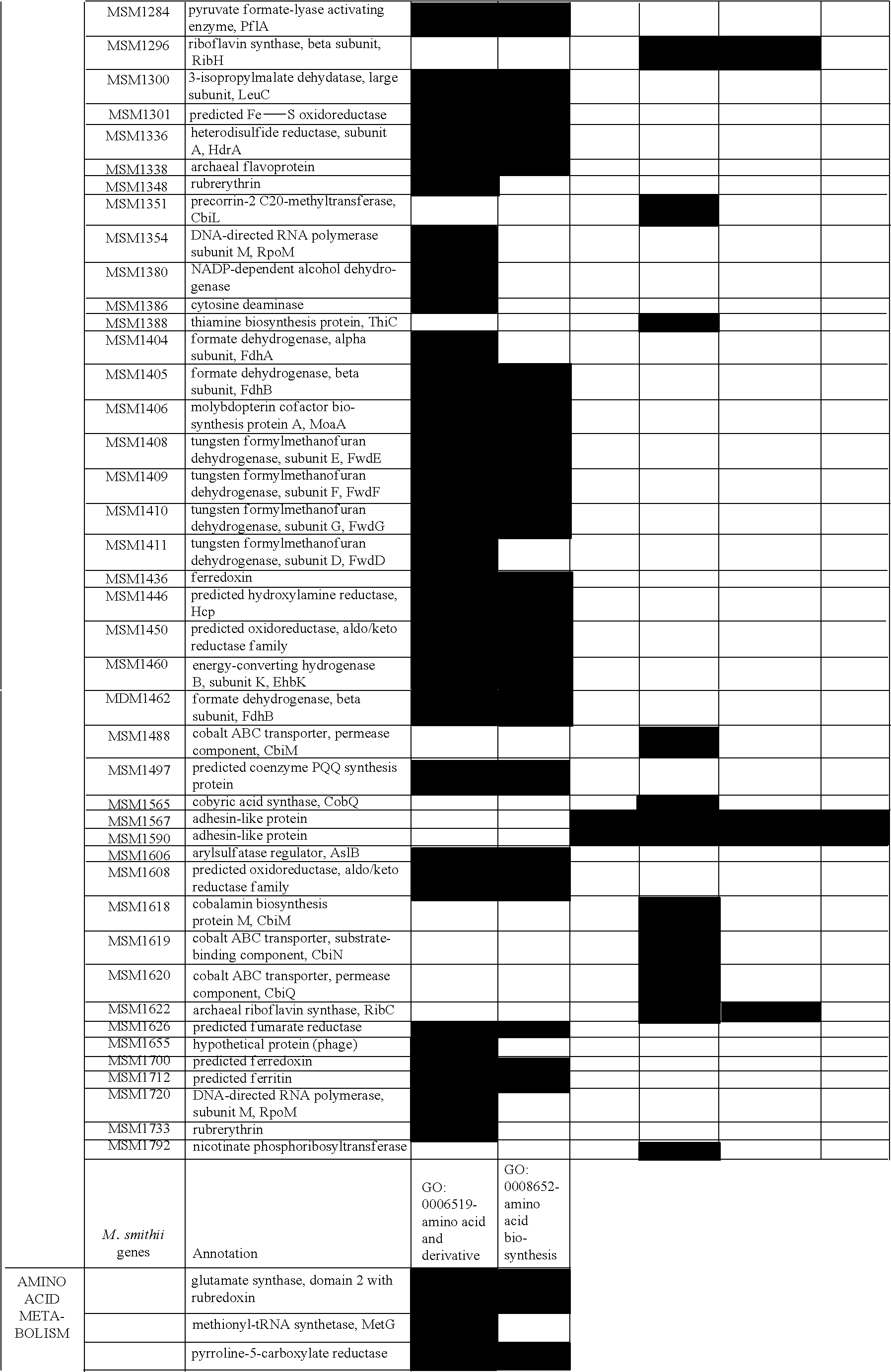

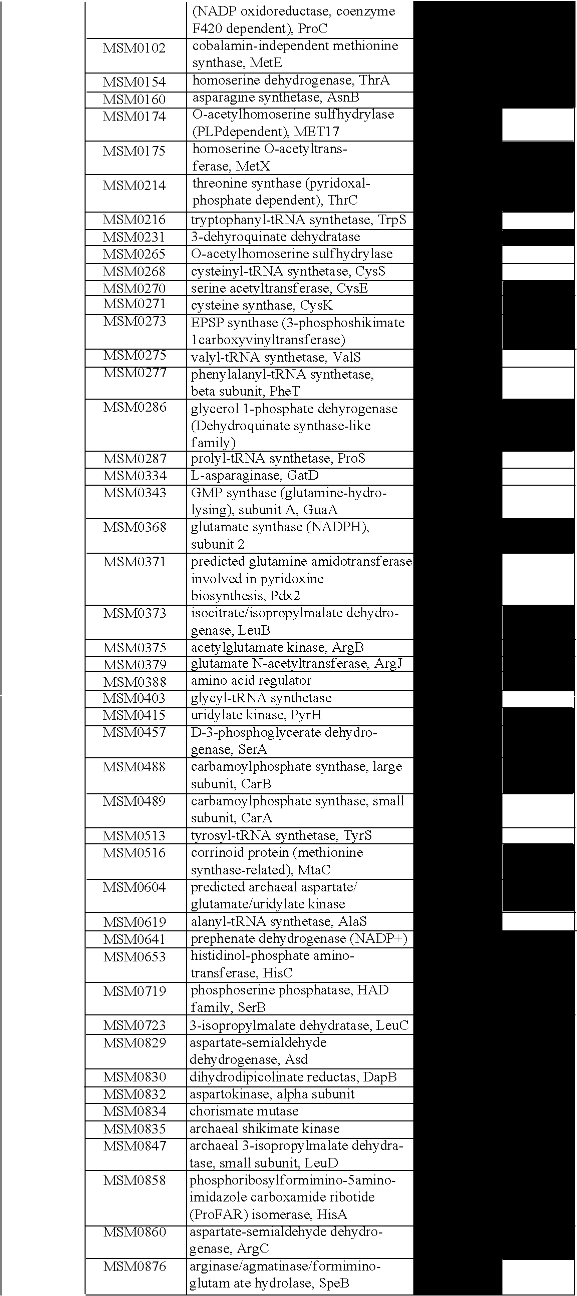

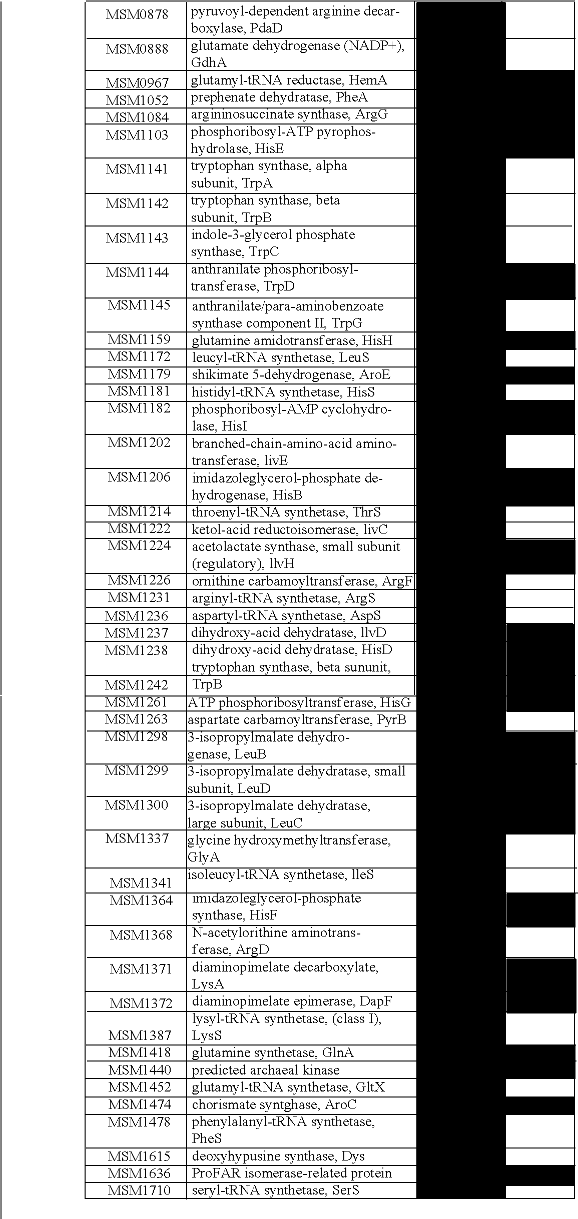

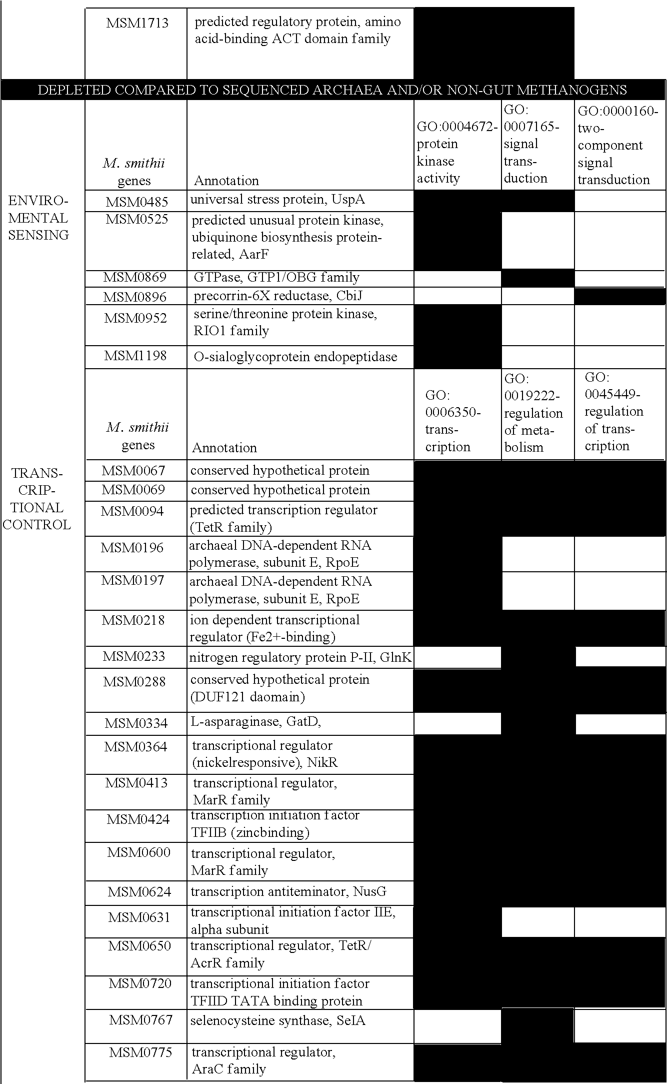

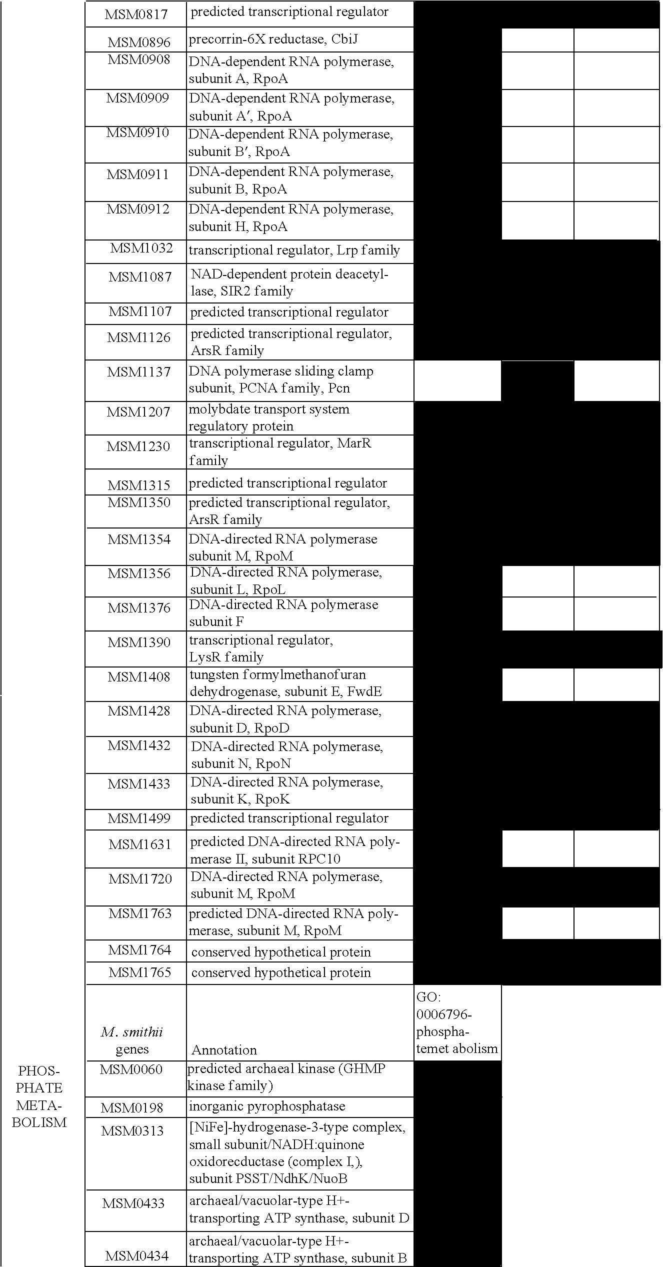

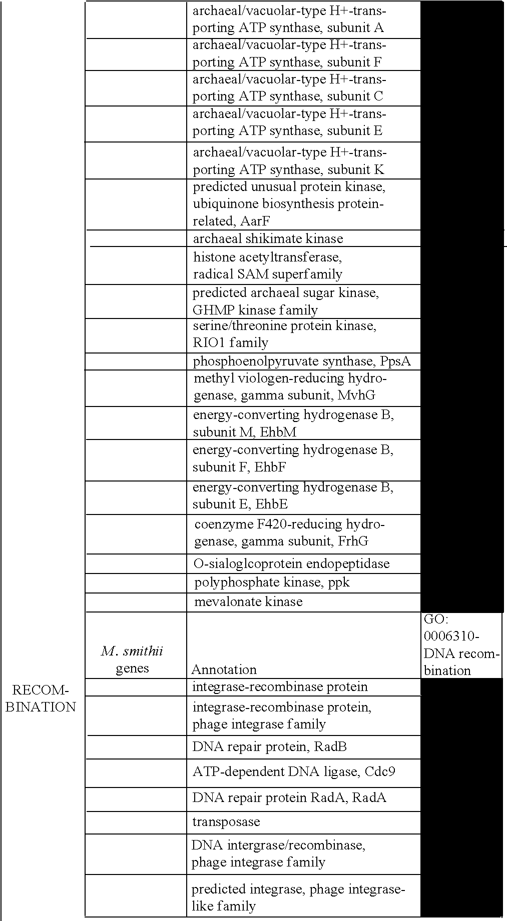

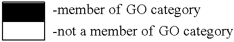

TABLE 8
M. smithii proteins with homologs in other sequenced Methanobacteriales

| | | | | Methanothermobacter | | |
|---|---|---|---|---|---|---|
| M. smithii | Methanosphaera stadtmanae | | | thermoautotrophicus | | |
| ORF | ORF | ANNOTATION | E-value | ORF | ANNOTATION | E-value |
| MSM0001 | Msp_0220 | predicted glycosyltransferase | 4.2E−08 | NONE | | |
| MSM0002 | Msp_1355 | predicted site-specific | 2.0E−08 | MTH_893 | integrase-recombinase | 8.1E−16 |
| | | recombinase/integrase | | | protein | |
| MSM0003 | Msp_0548 | hypothetical membrane-spanning | 6.8E−09 | NONE | | |
| | | protein | | | | |
| MSM0004 | Msp_0803 | conserved hypothetical protein | 2.3E−24 | NONE | | |
| MSM0005 | Msp_0783 | hypothetical membrane-spanning | 3.7E−05 | MTH_1439 | unknown | 6.2E−04 |
| | | protein | | | | |
| MSM0006 | Msp_0725 | hypothetical protein | 1.3E−05 | MTH_1277 | unknown | 3.3E−05 |
| MSM0007 | NONE | | | MTH_675 | unknown | 1.1E−34 |
| MSM0008 | Msp_0017 | conserved hypothetical protein | 1.7E−28 | NONE | | |
| MSM0009 | NONE | | | MTH_675 | unknown | 8.1E−34 |
| MSM0010 | Msp_0813 | conserved hypothetical protein | 1.5E−36 | MTH_676 | unknown | 1.7E−40 |
| MSM0011 | NONE | | | NONE | | |
| MSM0012 | Msp_0317 | hypothetical protein | 3.3E−04 | NONE | | |
| MSM0013 | NONE | | | NONE | | |
| MSM0014 | NONE | | | MTH_1289 | heat shock protein GrpE | 2.6E−04 |
| MSM0015 | NONE | | | NONE | | |
| MSM0016 | NONE | | | NONE | | |
| MSM0017 | NONE | | | NONE | | |
| MSM0018 | NONE | | | NONE | | |
| MSM0019 | NONE | | | NONE | | |
| MSM0020 | Msp_1323 | conserved hypothetical protein | 1.4E−05 | MTH_83 | O-linked GlcNAc | 3.3E−07 |
| | | | | | transferase | |
| MSM0021 | Msp_0047 | predicted short chain | 3.7E−40 | NONE | | |
| | | dehydrogenase | | | | |
| MSM0022 | NONE | | | NONE | | |
| MSM0023 | Msp_0424 | conserved hypothetical protein | 1.6E−25 | MTH_1084 | conserved protein | 4.4E−18 |
| MSM0024 | NONE | | | NONE | | |
| MSM0025 | Msp_0447 | predicted acyl-CoA synthetase | 3.7E−49 | MTH_657 | long-chain-fatty-acid-CoA | 8.7E−227 |
| | | | | | ligase | |
| MSM0026 | Msp_0265 | conserved hypothetical protein | 2.0E−16 | MTH_659 | epoxidase | 4.1E−62 |
| MSM0027 | Msp_0667 | putative glutamate synthase, | 7.9E−70 | NONE | glutamate synthase | 4.6E−79 |
| | | subunit 2 with ferredoxin domain | | | (NADPH), alpha subunit | |
| MSM0028 | Msp_0602 | conserved hypothetical protein | 1.9E−13 | MTH_1876 | conserved protein | 1.7E−04 |
| MSM0029 | NONE | | | NONE | | |
| MSM0030 | Msp_0741 | conserved hypothetical | 1.8E−72 | MTH_1812 | conserved protein | 1.6E−44 |
| | | membrane-spanning protein | | | | |
| MSM0031 | Msp_1465 | member of asn/thr-rich large | 2.9E−23 | MTH_716 | cell surface glycoprotein | 3.7E−04 |
| | | protein family | | | (s-layer protein) | |
| MSM0032 | NONE | | | NONE | | |
| MSM0033 | Msp_0966 | putative 2-dehydropantoate 2- | 6.8E−112 | NONE | | |
| | | reductase | | | | |
| MSM0034 | Msp_0725 | hypothetical protein | 7.9E−06 | NONE | | |
| MSM0035 | NONE | | | NONE | | |
| MSM0036 | NONE | | | NONE | | |
| MSM0037 | NONE | | | NONE | | |
| MSM0038 | NONE | | | NONE | | |
| MSM0039 | NONE | | | NONE | | |
| MSM0040 | Msp_1274 | conserved hypothetical protein | 5.5E−05 | NONE | | |
| MSM0041 | NONE | | | NONE | | |
| MSM0042 | NONE | | | NONE | | |
| MSM0043 | Msp_0737 | putative peptide methionine | 1.6E−32 | MTH_535 | peptide methionine | 5.3E−16 |
| | | sulfoxide reductase MsrA/MsrB | | | sulfoxide reductase | |
| MSM0044 | Msp_0510 | putative aspartate | 2.0E−15 | MTH_1894 | aspartate | 3.9E−13 |
| | | aminotransferase | | | aminotransferase | |
| | | | | | homolog | |
| MSM0045 | Msp_0283 | predicted ATPase | 3.9E−93 | MTH_1176 | nucleotide-binding protein | 1.4E−70 |
| | | | | | (putative ATPase) | |
| MSM0046 | Msp_1460 | predicted NAD(FAD)-dependent | 8.4E−114 | MTH_1354 | NADH oxidase | 2.0E−149 |
| | | dehydrogenase | | | | |
| MSM0047 | NONE | | | NONE | | |
| MSM0048 | Msp_0701 | hypothetical protein | 4.0E−20 | NONE | | |
| MSM0049 | Msp_0665 | F420H2:NADP oxidoreductase | 3.1E−75 | MTH_248 | conserved protein | 9.4E−56 |
| MSM0050 | Msp_1172 | conserved hypothetical protein | 1.7E−21 | NONE | | |
| MSM0051 | Msp_1399 | member of asn/thr-rich large | 4.0E−33 | MTH_716 | cell surface glycoprotein | 3.9E−11 |
| | | protein family | | | (s-layer protein) | |
| MSM0052 | Msp_0145 | member of asn/thr-rich large | 1.4E−53 | MTH_716 | cell surface glycoprotein | 1.8E−11 |
| | | protein family | | | (s-layer protein) | |
| MSM0053 | Msp_0086 | putative tRNA | 5.0E−100 | MTH_584 | tRNA | 2.5E−110 |
| | | nucleotidyltransferase | | | nucleotidyltransferase | |
| MSM0054 | Msp_0089 | predicted 2′-5′ RNA ligase | 7.2E−37 | MTH_583 | conserved protein | 9.1E−42 |
| MSM0055 | Msp_0090 | predicted 3-dehydroquinate | 3.5E−108 | MTH_580 | conserved protein | 3.3E−124 |
| | | synthase | | | | |
| MSM0056 | Msp_0091 | predicted fructose-bisphosphate | 1.5E−100 | MTH_579 | conserved protein | 2.9E−100 |
| | | aldolase | | | | |
| MSM0057 | Msp_0762 | member of asn/thr-rich large | 1.7E−13 | MTH_716 | cell surface glycoprotein | 8.2E−07 |
| | | protein family | | | (s-layer protein) | |
| MSM0058 | Msp_0128 | predicted helicase | 8.6E−23 | MTH_472 | DNA helicase II | 1.2E−90 |
| MSM0059 | Msp_0092 | conserved hypothetical protein | 9.4E−35 | MTH_578 | unknown | 2.1E−49 |
| MSM0060 | Msp_1187 | predicted archaeal kinase | 8.2E−52 | MTH_577 | conserved protein | 2.1E−49 |
| MSM0061 | Msp_0757 | predicted ATPase | 7.5E−97 | NONE | | |
| MSM0062 | Msp_0554 | hypothetical protein | 2.2E−08 | MTH_847 | unknown | 6.9E−08 |
| MSM0063 | Msp_1186 | predicted hydrolase | 1.3E−67 | MTH_576 | conserved protein | 7.0E−51 |
| MSM0064 | Msp_0099 | conserved hypothetical protein | 4.6E−10 | MTH_812 | conserved protein | 1.5E−09 |
| MSM0065 | Msp_1185 | putative 5-amino-6-(5- | 2.6E−55 | MTH_235 | riboflavin-specific | 1.5E−66 |
| | | phosphoribosylamino)uracil | | | deaminase | |
| | | reductase | | | | |
| MSM0066 | Msp_0080 | predicted glycosyltransferase | 8.2E−107 | MTH_590 | N-acetylglucosamine-1- | 7.9E−107 |
| | | | | | phosphate transferase | |
| MSM0067 | NONE | | | NONE | | |
| MSM0068 | Msp_0407 | conserved hypothetical protein | 6.0E−04 | MTH_521 | unknown | 8.4E−04 |
| MSM0069 | Msp_0081 | conserved hypothetical protein | 2.8E−26 | MTH_589 | conserved protein | 3.1E−25 |
| MSM0070 | Msp_0082 | conserved hypothetical protein | 2.8E−99 | MTH_588 | conserved protein | 4.8E−100 |
| MSM0071 | Msp_0083 | MetG | 5.3E−199 | MTH_587 | methionyl-tRNA | 2.9E−235 |
| | | | | | synthetase | |
| MSM0072 | Msp_0216 | hypothetical membrane-spanning | 2.2E−04 | NONE | | |
| | | protein | | | | |
| MSM0073 | Msp_0084 | DNA primase, large subunit | 1.4E−102 | MTH_586 | unknown | 1.7E−118 |
| MSM0074 | NONE | | | NONE | | |
| MSM0075 | Msp_0085 | DNA primase, small subunit | 1.2E−96 | NONE | DNA primase, small | 8.1E−105 |
| | | | | | subunit | |
| MSM0076 | Msp_0710 | hypothetical protein | 9.9E−04 | NONE | | |
| MSM0077 | Msp_0357 | putative thymidylate kinase | 6.9E−16 | MTH_1100 | conserved protein | 4.6E−47 |
| MSM0078 | NONE | | | MTH_1099 | conserved protein | 3.9E−50 |
| MSM0079 | Msp_0392 | CofH | 7.6E−81 | MTH_820 | conserved protein | 1.0E−106 |
| MSM0080 | Msp_0278 | ComD | 1.0E−53 | MTH_1206 | phosphonopyruvate | 1.7E−47 |
| | | | | | decarboxylase related | |
| | | | | | protein | |
| MSM0081 | Msp_0277 | ComE | 9.4E−51 | MTH_1207 | phosphonopyruvate | 1.7E−40 |
| | | | | | decarboxylase related | |
| | | | | | protein | |
| MSM0082 | Msp_0127 | HdrA2 | 1.3E−241 | NONE | heterodisulfide reductase, | 2.5E−133 |
| | | | | | subunit A | |
| MSM0083 | Msp_0126 | HdrB2 | 2.6E−94 | NONE | heterodisulfide reductase, | 8.6E−46 |
| | | | | | subunit B | |
| MSM0084 | Msp_0125 | HdrC2 | 2.6E−48 | NONE | heterodisulfide reductase, | 3.5E−17 |
| | | | | | subunit C | |
| MSM0085 | Msp_1261 | conserved hypothetical protein | 6.6E−114 | MTH_1684 | conserved protein | 2.1E−115 |
| | | | | | (contains ferredoxin | |
| | | | | | domain) | |
| MSM0086 | Msp_1270 | ComA | 5.2E−73 | MTH_1674 | conserved protein | 3.5E−81 |
| MSM0087 | Msp_0233 | conserved hypothetical protein | 2.3E−22 | NONE | | |
| MSM0088 | Msp_1322 | conserved hypothetical protein | 7.3E−44 | MTH_727 | conserved protein | 1.6E−51 |
| MSM0089 | Msp_1314 | ProC | 8.2E−07 | NONE | | |
| MSM0090 | NONE | | | MTH_224 | conserved protein | 8.6E−30 |
| MSM0091 | Msp_0129 | putative 2,3-diphosphoglycerate | 8.6E−144 | MTH_223 | unknown | 2.0E−172 |
| | | synthase | | | | |
| MSM0092 | Msp_0154 | member of asn/thr-rich large | 5.6E−08 | NONE | | |
| | | protein family | | | | |
| MSM0093 | Msp_1068 | partially conserved hypothetical | 1.1E−58 | MTH_1858 | phage infection protein | 5.7E−98 |
| | | membrane-spanning protein | | | homolog | |
| MSM0094 | Msp_0971 | hypothetical protein | 4.4E−09 | MTH_1787 | conserved protein | 9.3E−17 |
| MSM0095 | Msp_1181 | predicted phosphotransacetylase | 1.3E−44 | MTH_231 | conserved protein | 8.8E−44 |
| MSM0096 | Msp_1182 | UppS | 2.6E−96 | MTH_232 | conserved protein | 2.3E−100 |
| MSM0097 | Msp_1183 | predicted DNase | 3.2E−57 | MTH_233 | conserved protein | 3.4E−67 |
| MSM0098 | NONE | | | NONE | | |
| MSM0099 | Msp_0079 | hypothetical membrane-spanning | 2.1E−23 | MTH_596 | unknown | 8.2E−25 |
| | | protein | | | | |
| MSM0100 | Msp_0078 | hypothetical membrane-spanning | 7.3E−12 | MTH_429 | unknown | 1.1E−13 |
| | | protein | | | | |
| MSM0101 | Msp_0988 | CbiF | 9.8E−88 | MTH_602 | precorrin-3 methylase | 1.5E−80 |
| MSM0102 | Msp_1236 | MetE | 3.4E−69 | MTH_775 | cobalamin-independent | 3.8E−75 |
| | | | | | methionine synthase | |
| MSM0103 | NONE | | | MTH_776 | conserved protein | 7.3E−33 |
| MSM0104 | NONE | | | MTH_777 | conserved protein | 2.7E−42 |
| MSM0105 | Msp_1234 | conserved hypothetical | 3.8E−86 | MTH_778 | unknown | 5.9E−118 |
| | | membrane-spanning protein | | | | |
| MSM0106 | Msp_1232 | conserved hypothetical protein | 1.8E−109 | MTH_781 | conserved protein | 2.3E−132 |
| MSM0107 | Msp_1231 | HypB | 1.4E−79 | MTH_782 | hydrogenase | 1.1E−84 |
| | | | | | expression/formation | |
| | | | | | protein HypB | |
| MSM0108 | Msp_1230 | HypA | 5.8E−35 | MTH_783 | hydrogenase | 4.8E−36 |
| | | | | | expression/formation | |
| | | | | | protein HypA | |
| MSM0109 | Msp_0987 | hypothetical membrane-spanning | 8.6E−09 | NONE | | |
| | | protein | | | | |
| MSM0110 | Msp_0017 | conserved hypothetical protein | 1.5E−22 | NONE | | |
| MSM0111 | NONE | | | NONE | | |
| MSM0112 | Msp_0367 | predicted helicase | 1.2E−208 | NONE | ATP-dependent RNA | 1.4E−235 |
| | | | | | helicase, eIF-4A family | |
| MSM0113 | Msp_0128 | predicted helicase | 9.9E−137 | MTH_472 | DNA helicase II | 6.1E−26 |
| MSM0114 | NONE | | | NONE | | |
| MSM0115 | Msp_1290 | conserved hypothetical protein | 8.0E−29 | MTH_526 | conserved protein | 2.1E−51 |
| MSM0116 | Msp_1289 | conserved hypothetical protein | 3.5E−51 | MTH_528 | unknown | 9.1E−42 |
| MSM0117 | Msp_1288 | conserved hypothetical | 4.7E−56 | MTH_529 | unknown | 1.5E−66 |
| | | membrane-spanning protein | | | | |
| MSM0118 | Msp_1286 | conserved hypothetical protein | 1.1E−86 | MTH_532 | UDP-N-acetylmuramyl | 2.9E−86 |
| | | | | | tripeptide synthetase | |
| | | | | | related protein | |
| MSM0119 | Msp_0156 | predicted nuclease | 3.2E−18 | MTH_538 | unknown | 2.5E−14 |
| MSM0120 | Msp_1095 | DNA double-strand break repair | 1.3E−92 | MTH_540 | intracellular protein | 2.1E−27 |
| | | protein Rad50 | | | transport protein | |
| MSM0121 | Msp_1094 | DNA double-strand break repair | 3.7E−72 | MTH_541 | Rad32 related protein | 1.2E−16 |
| | | protein Mre11 | | | | |
| MSM0122 | Msp_1093 | predicted ATPase | 1.7E−122 | MTH_307 | conserved protein | 4.2E−124 |
| MSM0123 | Msp_1092 | conserved hypothetical protein | 2.4E−29 | MTH_306 | conserved protein | 1.2E−32 |
| MSM0124 | Msp_1291 | PcrB | 5.1E−75 | MTH_552 | conserved protein | 2.9E−84 |
| MSM0125 | Msp_1292 | 50S ribosomal protein L40e | 5.5E−23 | MTH_553 | ribosomal protein L40 | 7.6E−22 |
| MSM0126 | Msp_1293 | conserved hypothetical protein | 9.4E−51 | MTH_554 | conserved protein | 2.9E−54 |
| MSM0127 | NONE | | | NONE | | |
| MSM0128 | Msp_0853 | conserved hypothetical | 2.3E−10 | MTH_570 | unknown | 2.8E−31 |
| | | membrane-spanning protein | | | | |
| MSM0129 | Msp_0435 | nicotinamide-nucleotide | 8.1E−61 | MTH_150 | conserved protein | 6.7E−62 |
| | | adenylyltransferase | | | | |
| MSM0130 | NONE | | | MTH_149 | molybdenum cofactor | 6.6E−39 |
| | | | | | biosynthesis protein MoaE | |
| MSM0131 | NONE | | | MTH_920 | anion permease | 1.5E−04 |
| MSM0132 | NONE | | | MTH_1797 | conserved protein | 7.9E−20 |
| MSM0133 | Msp_1198 | predicted thioesterase | 2.2E−42 | MTH_658 | unknown | 4.8E−36 |
| MSM0134 | Msp_0565 | predicted M42 glutamyl | 2.2E−115 | NONE | endo-1,4-beta-glucanase | 3.7E−116 |
| | | aminopeptidase | | | | |
| MSM0135 | Msp_0668 | conserved hypothetical protein | 9.1E−85 | NONE | coenzyme F420-reducing | 4.5E−88 |
| | | | | | hydrogenase, beta | |
| | | | | | subunit homolog | |
| MSM0136 | Msp_0147 | ferredoxin | 2.2E−06 | NONE | tungsten | 2.2E−06 |
| | | | | | formylmethanofuran | |
| | | | | | dehydrogenase, subunit G | |
| MSM0137 | Msp_0220 | predicted glycosyltransferase | 3.7E−12 | MTH_540 | intracellular protein | 4.7E−05 |
| | | | | | transport protein | |
| MSM0138 | NONE | | | MTH_491 | conserved protein | 2.6E−51 |
| MSM0139 | Msp_0448 | predicted polysaccharide | 7.6E−04 | NONE | | |
| | | biosynthesis protein | | | | |
| MSM0140 | Msp_0560 | conserved hypothetical protein | 4.0E−59 | MTH_435 | conserved protein | 2.9E−68 |
| MSM0141 | Msp_0561 | predicted dephospho-CoA kinase | 5.5E−23 | MTH_434 | UMP/CMP kinase related | 5.6E−42 |
| | | | | | protein | |
| MSM0142 | Msp_0563 | predicted ATPase of PP-loop | 3.2E−66 | MTH_432 | conserved protein | 2.9E−68 |
| | | superfamily | | | | |
| MSM0143 | Msp_0564 | partially conserved hypothetical | 1.3E−30 | MTH_431 | unknown | 2.4E−34 |
| | | membrane-spanning protein | | | | |
| MSM0144 | NONE | | | NONE | | |
| MSM0145 | Msp_0451 | hypothetical membrane-spanning | 1.9E−13 | MTH_422 | unknown | 1.6E−14 |
| | | protein | | | | |
| MSM0146 | Msp_0452 | conserved hypothetical | 7.0E−18 | MTH_421 | unknown | 2.0E−21 |
| | | membrane-spanning protein | | | | |
| MSM0147 | Msp_0453 | PyrG | 2.2E−202 | MTH_419 | CTP synthase | 2.9E−212 |
| MSM0148 | Msp_0739 | predicted oxidoreductase | 3.9E−93 | MTH_907 | conserved protein | 3.1E−32 |
| MSM0149 | NONE | | | NONE | | |
| MSM0150 | NONE | | | NONE | | |
| MSM0151 | NONE | | | NONE | | |
| MSM0152 | Msp_1417 | predicted Na+-driven multidrug | 1.1E−28 | MTH_314 | conserved protein | 4.7E−23 |
| | | efflux pump | | | | |
| MSM0153 | Msp_0485 | ApgM1 | 1.3E−110 | MTH_418 | phosphonopyruvate | 2.1E−106 |
| | | | | | decarboxylase related | |
| | | | | | protein | |
| MSM0154 | Msp_0487 | putative homoserine | 1.3E−101 | MTH_417 | homoserine | 6.1E−100 |
| | | dehydrogenase | | | dehydrogenase homolog | |
| MSM0155 | Msp_0488 | predicted allosteric regulator of | 1.1E−29 | MTH_416 | conserved protein | 7.8E−36 |
| | | homoserine dehydrogenase | | | | |
| MSM0156 | Msp_0489 | conserved hypothetical protein | 2.6E−23 | MTH_415 | conserved protein | 3.3E−21 |
| MSM0157 | Msp_0484 | predicted type I restriction- | 1.9E−09 | NONE | type I restriction | 5.3E−09 |
| | | modification system subunit | | | modification system, | |
| | | | | | subunit S | |
| MSM0158 | Msp_0483 | hypothetical protein | 2.3E−17 | NONE | type I restriction | 2.2E−13 |
| | | | | | modification system, | |
| | | | | | subunit S | |
| MSM0159 | Msp_0777 | member of asn/thr-rich large | 2.1E−13 | NONE | | |
| | | protein family | | | | |
| MSM0160 | Msp_0490 | putative asparagine synthetase | 7.9E−102 | MTH_414 | asparagine synthetase | 2.3E−91 |
| MSM0161 | NONE | | | NONE | | |
| MSM0162 | NONE | | | NONE | | |
| MSM0163 | Msp_0425 | conserved hypothetical protein | 7.0E−23 | MTH_1083 | conserved protein | 5.6E−26 |
| MSM0164 | Msp_0946 | conserved hypothetical protein | 1.3E−106 | MTH_1084 | conserved protein | 4.6E−118 |
| MSM0165 | Msp_0945 | predicted RecB family | 7.9E−54 | MTH_1085 | conserved protein | 1.8E−45 |
| | | exonuclease | | | | |
| MSM0166 | Msp_0422 | predicted helicase | 2.3E−27 | MTH_1086 | conserved protein | 9.1E−32 |
| MSM0167 | NONE | | | MTH_1087 | unknown | 8.4E−04 |
| MSM0168 | NONE | | | NONE | | |
| MSM0169 | Msp_0220 | predicted glycosyltransferase | 2.1E−04 | NONE | | |
| MSM0170 | Msp_0944 | conserved hypothetical protein | 1.4E−63 | MTH_1091 | conserved protein | 3.4E−35 |
| MSM0171 | Msp_0835 | hypothetical membrane-spanning | 2.7E−43 | MTH_769 | unknown | 1.7E−34 |
| | | protein | | | | |
| MSM0172 | NONE | | | NONE | | |
| MSM0173 | Msp_0145 | member of asn/thr-rich large | 3.2E−34 | MTH_1074 | putative membrane | 5.5E−31 |
| | | protein family | | | protein | |
| MSM0174 | Msp_0677 | predicted O-acetylhomoserine | 1.9E−123 | NONE | | |
| | | sulfhydrylase | | | | |
| MSM0175 | Msp_0676 | MetX | 2.3E−166 | MTH_1820 | homoserine O- | 1.5E−21 |
| | | | | | acetyltransferase | |
| MSM0176 | NONE | | | NONE | | |
| MSM0177 | NONE | | | NONE | | |
| MSM0178 | Msp_1385 | conserved hypothetical protein | 1.5E−27 | NONE | | |
| MSM0179 | NONE | | | NONE | | |
| MSM0180 | NONE | | | MTH_698 | unknown | 1.6E−04 |
| MSM0181 | Msp_1174 | 50S ribosomal protein L37e | 9.6E−26 | MTH_648 | ribosomal protein L37 | 2.8E−24 |
| MSM0182 | Msp_1175 | putative snRNP Sm-like protein | 1.5E−27 | MTH_649 | conserved protein | 2.1E−33 |
| MSM0183 | Msp_1176 | predicted RNA-binding protein | 9.0E−46 | MTH_650 | conserved protein | 8.6E−46 |
| MSM0184 | Msp_1177 | predicted creatinine | 1.3E−51 | MTH_651 | conserved protein | 1.6E−51 |
| | | amidohydrolase | | | | |
| MSM0185 | Msp_0547 | hypothetical membrane-spanning | 7.8E−08 | MTH_515 | unknown | 4.3E−05 |
| | | protein | | | | |
| MSM0186 | Msp_0345 | conserved hypothetical protein | 1.3E−14 | NONE | | |
| MSM0187 | Msp_0444 | rubredoxin | 2.5E−09 | MTH_156 | rubredoxin | 2.3E−13 |
| MSM0188 | Msp_0444 | rubredoxin | 3.4E−14 | MTH_156 | rubredoxin | 3.5E−17 |
| MSM0189 | Msp_1301 | predicted nucleoside- | 4.6E−08 | MTH_272 | acetyl/acyl transferase | 1.3E−58 |
| | | diphosphate-sugar | | | related protein | |
| | | pyrophosphorylase | | | | |
| MSM0190 | Msp_0617 | predicted ATPase | 3.1E−84 | MTH_271 | conserved protein | 1.8E−75 |
| MSM0191 | Msp_1533 | RpoM1 | 1.5E−04 | NONE | | |
| MSM0192 | Msp_0618 | ArgH | 2.7E−147 | MTH_269 | argininosuccinate lyase | 8.2E−160 |
| MSM0193 | Msp_0620 | 30S ribosomal protein S27Ae | 1.8E−17 | MTH_268 | ribosomal protein S27a | 8.1E−18 |
| MSM0194 | Msp_0621 | 30S ribosomal protein S24e | 1.1E−26 | MTH_267 | ribosomal protein S24 | 1.6E−28 |
| MSM0195 | Msp_0622 | conserved hypothetical protein | 4.8E−31 | MTH_266 | conserved protein | 1.3E−33 |
| MSM0196 | Msp_0623 | RpoE2 | 9.0E−14 | NONE | DNA-dependent RNA | 1.5E−18 |
| | | | | | polymerase, subunit E″ | |
| MSM0197 | Msp_0624 | RpoE1 | 2.2E−65 | NONE | DNA-dependent RNA | 1.3E−67 |
| | | | | | polymerase, subunit E′ | |
| MSM0198 | Msp_0625 | inorganic pyrophosphatase | 3.1E−68 | MTH_263 | inorganic | 7.2E−65 |
| | | | | | pyrophosphatase | |
| MSM0199 | Msp_0626 | conserved hypothetical protein | 2.4E−22 | MTH_262 | conserved protein | 3.7E−29 |
| MSM0200 | Msp_0627 | putative translation initiation factor | 3.3E−158 | NONE | translation initiation factor | 1.6E−163 |
| | | 2, subunit gamma (aIF- | | | eIF-2, gamma subunit | |
| | | 2gamma)(eIF2G) | | | | |
| MSM0201 | Msp_0628 | 30S ribosomal protein S6e | 9.9E−40 | MTH_260 | ribosomal protein S6 | 1.5E−41 |
| MSM0202 | Msp_0629 | InfB | 9.3E−202 | MTH_259 | translation initiation factor | 2.6E−218 |
| | | | | | IF2 homolog | |
| MSM0203 | Msp_0630 | nucleoside diphosphate kinase | 1.8E−56 | MTH_258 | nucleoside diphosphate | 1.9E−57 |
| | | | | | kinase | |
| MSM0204 | Msp_0631 | 50S ribosomal protein L24e | 3.0E−22 | MTH_257 | ribosomal protein L24 | 8.2E−25 |
| MSM0205 | Msp_0632 | 30S ribosomal protein S28e | 4.3E−30 | MTH_256 | ribosomal protein S28 | 2.2E−31 |
| MSM0206 | Msp_0633 | 50S ribosomal protein L7Ae | 9.3E−44 | MTH_255 | ribosomal protein L7a | 1.3E−44 |
| MSM0207 | NONE | | | MTH_1178 | conserved protein | 1.9E−41 |
| MSM0208 | NONE | | | MTH_1178 | conserved protein | 3.9E−08 |
| MSM0209 | Msp_0861 | ferredoxin | 7.3E−12 | MTH_1106 | ferredoxin | 7.6E−22 |
| MSM0210 | Msp_0253 | conserved hypothetical | 1.1E−04 | NONE | | |
| | | membrane-spanning protein | | | | |
| MSM0211 | NONE | | | NONE | | |
| MSM0212 | NONE | | | NONE | | |
| MSM0213 | Msp_0769 | archaeal histone | 8.2E−20 | MTH_821 | histone HMtA1 | 3.7E−22 |
| MSM0214 | Msp_0588 | ThrC | 2.0E−153 | MTH_253 | threonine synthase | 8.8E−163 |
| MSM0215 | Msp_0232 | hypothetical membrane-spanning | 2.4E−22 | MTH_252 | conserved protein | 4.5E−24 |
| | | protein | | | | |
| MSM0216 | Msp_0653 | TrpS | 5.0E−132 | MTH_251 | tryptophanyl-tRNA | 1.8E−116 |
| | | | | | synthetase | |
| MSM0217 | Msp_0652 | EndA | 5.0E−45 | MTH_250 | tRNA intron endonuclease | 2.7E−49 |
| MSM0218 | Msp_0446 | predicted metal-dependent | 5.3E−57 | MTH_214 | iron repressor | 6.4E−57 |
| | | transcriptional regulator | | | | |
| MSM0219 | Msp_1129 | partially conserved hypothetical | 1.0E−46 | MTH_357 | conserved protein | 4.0E−67 |
| | | membrane-spanning protein | | | | |
| MSM0220 | Msp_0114 | ThsB | 1.7E−170 | MTH_218 | chaperonin | 4.0E−183 |
| MSM0221 | Msp_0590 | member of asn/thr-rich large | 6.9E−13 | MTH_719 | cell surface glycoprotein | 4.2E−05 |
| | | protein family | | | (s-layer protein) | |
| MSM0222 | Msp_0787 | FprA | 2.5E−128 | MTH_220 | flavoprotein A homolog (II) | 3.2E−133 |
| MSM0223 | NONE | | | MTH_557 | unknown | 1.4E−22 |
| MSM0224 | NONE | | | MTH_558 | unknown | 2.1E−28 |
| MSM0225 | Msp_1294 | conserved hypothetical | 1.4E−47 | MTH_559 | conserved protein | 1.4E−54 |
| | | membrane-spanning protein | | | | |
| MSM0226 | NONE | | | NONE | | |
| MSM0227 | Msp_0584 | HmgA | 2.2E−138 | MTH_562 | 3-hydroxy-3- | 1.7E−143 |
| | | | | | methylglutaryl CoA | |
| | | | | | reductase | |
| MSM0228 | Msp_0583 | SucD | 1.7E−99 | NONE | succinyl-CoA synthetase, | 1.3E−111 |
| | | | | | alpha subunit | |
| MSM0229 | Msp_0582 | conserved hypothetical protein | 1.6E−69 | MTH_564 | conserved protein | 1.5E−87 |
| MSM0230 | Msp_0233 | conserved hypothetical protein | 2.9E−21 | NONE | | |
| MSM0231 | Msp_0577 | AroD | 9.9E−40 | MTH_566 | 3-dehydroquinate | 2.9E−52 |
| | | | | | dehydratase | |
| MSM0232 | Msp_0145 | member of asn/thr-rich large | 3.8E−05 | MTH_567 | unknown | 7.5E−31 |
| | | protein family | | | | |
| MSM0233 | Msp_0664 | nitrogen regulatory protein P-II | 7.9E−31 | MTH_664 | nitrogen regulatory protein | 1.4E−36 |
| | | | | | P-II | |
| MSM0234 | Msp_0663 | ammonium transporter | 4.8E−150 | MTH_663 | ammonium transporter | 1.2E−142 |
| MSM0235 | Msp_0119 | hypothetical membrane-spanning | 6.0E−04 | MTH_181 | unknown | 1.4E−04 |
| | | protein | | | | |
| MSM0236 | Msp_0434 | predicted phosphohydrolase | 1.2E−100 | MTH_148 | conserved protein | 7.8E−123 |
| MSM0237 | Msp_0088 | predicted 3-polyprenyl-4- | 3.1E−59 | MTH_147 | phenylacrylic acid | 2.6E−53 |
| | | hydroxybenzoate decarboxylase | | | decarboxylase | |
| MSM0238 | Msp_0087 | CbiT | 4.2E−48 | MTH_146 | precorrin-8W | 3.1E−48 |
| | | | | | decarboxylase | |
| MSM0239 | NONE | | | MTH_145 | conserved protein | 6.9E−44 |
| MSM0240 | Msp_1289 | conserved hypothetical protein | 8.3E−07 | MTH_143 | molybdopterin-guanine | 1.6E−30 |
| | | | | | dinucleotide biosynthesis | |
| | | | | | MobA related protein | |
| MSM0241 | Msp_1252 | putative exosome complex, | 1.1E−61 | MTH_682 | conserved protein | 5.6E−90 |
| | | exonuclease 2 subunit | | | | |
| MSM0242 | Msp_1251 | putative exosome complex, | 1.4E−79 | MTH_683 | ribonuclease PH | 1.1E−93 |
| | | exonuclease 1 subunit | | | | |
| MSM0243 | Msp_1250 | putative exosome complex, RNA- | 1.6E−48 | MTH_684 | conserved protein | 2.1E−90 |
| | | binding subunit | | | | |
| MSM0244 | Msp_1249 | conserved hypothetical protein | 1.8E−70 | MTH_685 | conserved protein | 8.3E−80 |
| MSM0245 | Msp_1248 | PsmA | 6.3E−77 | NONE | proteasome, alpha | 2.5E−94 |
| | | | | | subunit | |
| MSM0246 | Msp_1246 | putative ribonuclease P, | 1.3E−19 | MTH_687 | conserved protein | 2.3E−22 |
| | | component 2 | | | | |
| MSM0247 | Msp_1245 | putative ribonuclease P, | 2.1E−28 | MTH_688 | conserved protein | 3.1E−41 |
| | | component 3 | | | | |
| MSM0248 | Msp_0950 | hypothetical protein | 7.2E−05 | NONE | | |
| MSM0249 | Msp_1548 | hypothetical protein | 1.8E−04 | MTH_301 | unknown | 4.1E−23 |
| MSM0250 | Msp_0501 | hypothetical membrane-spanning | 1.0E−05 | MTH_521 | unknown | 3.6E−10 |
| | | protein | | | | |
| MSM0251 | Msp_0725 | hypothetical protein | 1.5E−04 | NONE | | |
| MSM0252 | Msp_0824 | predicted Na+-driven multidrug | 1.6E−96 | MTH_314 | conserved protein | 3.7E−93 |
| | | efflux pump | | | | |
| MSM0253 | NONE | | | MTH_1725 | unknown | 1.4E−15 |
| MSM0254 | NONE | | | NONE | | |
| MSM0255 | NONE | | | NONE | | |
| MSM0256 | Msp_0017 | conserved hypothetical protein | 1.7E−28 | NONE | | |
| MSM0257 | Msp_0975 | hypothetical membrane-spanning | 4.3E−30 | NONE | | |
| | | protein | | | | |
| MSM0258 | Msp_0724 | hypothetical membrane-spanning | 1.6E−04 | NONE | | |
| | | protein | | | | |
| MSM0259 | Msp_1548 | hypothetical protein | 1.1E−05 | MTH_521 | unknown | 6.8E−04 |
| MSM0260 | Msp_0507 | predicted archaea-specific RecJ- | 2.0E−199 | MTH_763 | conserved protein | 3.4E−225 |
| | | like exonuclease | | | | |
| MSM0261 | Msp_1384 | conserved hypothetical | 1.1E−04 | MTH_759 | unknown | 1.5E−16 |
| | | membrane-spanning protein | | | | |
| MSM0262 | Msp_0788 | desulfoferrodoxin | 1.4E−26 | MTH_757 | rubredoxin | 3.4E−26 |
| | | | | | oxidoreductase | |
| MSM0263 | Msp_1003 | predicted NifU protein | 1.1E−47 | NONE | | |
| MSM0264 | Msp_1002 | IscS | 6.6E−121 | MTH_1389 | nifS protein | 1.6E−30 |
| MSM0265 | Msp_0677 | predicted O-acetylhomoserine | 1.5E−148 | MTH_1188 | pleiotropic regulatory | 3.1E−04 |
| | | sulfhydrylase | | | protein DegT | |
| MSM0266 | Msp_0145 | member of asn/thr-rich large | 2.7E−50 | MTH_911 | probable surface protein | 6.2E−09 |
| | | protein family | | | | |
| MSM0267 | Msp_0844 | predicted multimeric flavodoxin | 4.4E−53 | MTH_135 | conserved protein | 2.7E−17 |
| MSM0268 | Msp_0124 | CysS | 1.2E−139 | MTH_587 | methionyl-tRNA | 9.6E−08 |
| | | | | | synthetase | |
| MSM0269 | Msp_0527 | conserved hypothetical protein | 8.0E−38 | NONE | | |
| MSM0270 | Msp_0450 | predicted serine acetyltransferase | 8.1E−61 | MTH_1588 | ferripyochelin binding | 2.0E−06 |
| | | | | | protein | |
| MSM0271 | Msp_0449 | cysteine synthase | 2.2E−97 | NONE | tryptophan synthase, beta | 3.1E−08 |
| | | | | | subunit | |
| MSM0272 | Msp_0497 | putative endonuclease III | 2.2E−67 | MTH_764 | endonuclease III | 1.1E−70 |
| MSM0273 | Msp_0498 | AroA | 1.1E−102 | MTH_766 | 5-enolpyruvylshikimate 3- | 2.5E−62 |
| | | | | | phosphate synthase | |
| MSM0274 | NONE | | | NONE | | |
| MSM0275 | Msp_0499 | ValS | 2.4E−235 | MTH_767 | valyl-tRNA synthetase | 0.0E+00 |
| MSM0276 | Msp_0526 | hypothetical membrane-spanning | 8.1E−29 | MTH_768 | unknown | 2.9E−22 |
| | | protein | | | | |
| MSM0277 | Msp_0525 | PheT | 3.3E−151 | MTH_770 | phenylalanyl-tRNA | 4.2E−172 |
| | | | | | synthetase | |
| MSM0278 | NONE | | | NONE | | |
| MSM0279 | Msp_0522 | conserved hypothetical protein | 4.0E−36 | MTH_771 | conserved protein | 2.7E−35 |
| MSM0280 | Msp_0757 | predicted ATPase | 4.4E−13 | NONE | | |
| MSM0281 | Msp_0145 | member of asn/thr-rich large | 2.1E−09 | MTH_911 | probable surface protein | 2.9E−10 |
| | | protein family | | | | |
| MSM0282 | Msp_0141 | member of asn/thr-rich large | 1.3E−23 | MTH_911 | probable surface protein | 1.1E−17 |
| | | protein family | | | | |
| MSM0283 | NONE | | | MTH_436 | unknown | 1.1E−04 |
| MSM0284 | Msp_0995 | RpiA | 5.8E−74 | MTH_608 | ribose 5-phosphate | 1.3E−74 |
| | | | | | isomerase | |
| MSM0285 | Msp_0996 | conserved hypothetical protein | 1.3E−28 | MTH_609 | conserved protein | 1.3E−35 |
| MSM0286 | Msp_0997 | EgsA | 7.9E−102 | MTH_610 | glycerol 1-phosphate | 1.5E−112 |
| | | | | | dehydrogenase | |
| MSM0287 | Msp_1004 | ProS | 8.6E−160 | MTH_611 | prolyl-tRNA synthetase | 1.4E−155 |
| MSM0288 | Msp_1006 | conserved hypothetical protein | 1.7E−53 | MTH_613 | conserved protein | 4.2E−60 |
| MSM0289 | Msp_1007 | ThiD | 3.6E−58 | MTH_614 | transcriptional regulator | 5.1E−64 |
| MSM0290 | Msp_1000 | predicted ABC-type | 2.6E−71 | MTH_920 | anion permease | 1.4E−31 |
| | | nitrate/sulfonate/bicarbonate | | | | |
| | | transport system, ATB-binding | | | | |
| | | protein | | | | |
| MSM0291 | Msp_1001 | predicted ABC-type | 1.9E−84 | MTH_1730 | phosphate transporter | 4.8E−07 |
| | | nitrate/sulfonate/bicarbonate | | | permease PstC homolog | |
| | | transport system, permease | | | | |
| | | protein | | | | |
| MSM0292 | NONE | | | NONE | | |
| MSM0293 | Msp_0826 | predicted cation transport ATPase | 1.8E−198 | MTH_1535 | heavy-metal transporting | 1.2E−69 |
| | | | | | CPx-type ATPase | |
| MSM0294 | Msp_0825 | hypothetical protein | 4.2E−09 | NONE | | |
| MSM0295 | NONE | | | NONE | nitrate assimilation | 7.1E−49 |
| | | | | | protein, narQ | |
| MSM0296 | NONE | | | MTH_691 | conserved protein | 1.2E−30 |
| MSM0297 | Msp_1244 | predicted exosome subunit | 1.1E−24 | MTH_689 | conserved protein | 2.7E−26 |
| MSM0298 | Msp_1243 | 50S ribosomal protein L15e | 2.1E−76 | MTH_690 | ribosomal protein L15 | 1.3E−67 |
| MSM0299 | NONE | | | NONE | | |
| MSM0300 | Msp_0851 | predicted ABC-type | 1.5E−139 | NONE | | |
| | | dipeptide/oligopeptide/nickel | | | | |
| | | transport system, solute-binding | | | | |
| | | protein | | | | |
| MSM0301 | Msp_0811 | ABC-type dipeptide transport | 2.3E−120 | NONE | | |
| | | system, permease protein | | | | |
| MSM0302 | Msp_0810 | ABC-type dipeptide transport | 1.7E−99 | MTH_1729 | phosphate transporter | 2.3E−05 |
| | | system, permease protein | | | permease PstC | |
| MSM0303 | Msp_0848 | predicted ABC-type | 3.4E−101 | MTH_696 | ABC transporter | 1.4E−20 |
| | | dipeptide/oligopeptide/nickel | | | (glutamine transport ATP- | |
| | | transport system, ATP-binding | | | binding protein) | |
| | | protein | | | | |
| MSM0304 | Msp_0847 | predicted ABC-type | 4.8E−63 | NONE | methyl coenzyme M | 7.3E−21 |
| | | dipeptide/oligopeptide/nickel | | | reductase system, | |
| | | transport system, ATP-binding | | | component A2 | |
| | | protein | | | | |
| MSM0305 | Msp_0431 | GuaB | 6.1E−10 | MTH_406 | conserved protein | 7.6E−70 |
| MSM0306 | Msp_1447 | EhbK | 3.0E−18 | MTH_405 | polyferredoxin | 1.6E−37 |
| MSM0307 | Msp_0071 | predicted ribokinase | 3.4E−62 | MTH_404 | ribokinase | 3.5E−65 |
| MSM0308 | Msp_0070 | formylmethanofuran- | 6.7E−89 | MTH_403 | formylmethanofuran:tetrahydro- | 1.7E−95 |
| | | tetrahydromethanopterin | | | methanopterin formyltransferase II | |
| | | formyltransferase | | | | |
| MSM0309 | Msp_0069 | conserved hypothetical | 2.4E−68 | MTH_402 | unknown | 3.9E−57 |
| | | membrane-spanning protein | | | | |
| MSM0310 | Msp_1447 | EhbK | 1.7E−23 | MTH_401 | polyferredoxin | 7.7E−77 |
| MSM0311 | Msp_1447 | EhbK | 2.1E−13 | MTH_399 | polyferredoxin | 7.4E−111 |
| MSM0312 | Msp_1444 | EhbN | 2.2E−51 | NONE | formate hydrogenlyase, | 7.8E−139 |
| | | | | | subunit 5 | |
| MSM0313 | Msp_1445 | EhbM | 5.4E−32 | NONE | formate hydrogenlyase, | 6.3E−66 |
| | | | | | subunit 7 | |
| MSM0314 | NONE | | | MTH_396 | conserved protein | 2.9E−29 |
| MSM0315 | NONE | | | MTH_395 | conserved protein | 1.9E−18 |
| MSM0316 | Msp_0616 | partially conserved hypothetical | 9.5E−04 | MTH_394 | unknown | 5.8E−08 |
| | | membrane-spanning protein | | | | |
| MSM0317 | Msp_1443 | EhbO | 1.1E−16 | NONE | NADH dehydrogenase | 1.9E−105 |
| | | | | | (ubiquinone), subunit 1 | |
| | | | | | related protein | |
| MSM0318 | NONE | | | MTH_392 | unknown | 1.4E−15 |
| MSM0319 | Msp_1452 | EhbF | 4.0E−06 | NONE | NADH dehydrogenase I, | 5.5E−83 |
| | | | | | subunit N related protein | |
| MSM0320 | NONE | | | MTH_390 | conserved protein | 7.0E−67 |
| MSM0321 | NONE | | | MTH_389 | conserved protein | 6.6E−55 |
| MSM0322 | NONE | | | MTH_388 | unknown | 1.5E−25 |
| MSM0323 | NONE | | | MTH_387 | conserved protein | 3.9E−18 |
| MSM0324 | NONE | | | MTH_386 | unknown | 6.4E−18 |
| MSM0325 | NONE | | | MTH_385 | conserved protein | 4.1E−55 |
| MSM0326 | NONE | | | MTH_384 | unknown | 3.5E−17 |
| MSM0327 | Msp_0067 | putative UDP-glucose 4- | 1.2E−73 | MTH_380 | UDP-glucose 4-epimerase | 1.7E−86 |
| | | epimerase | | | homolog | |
| MSM0328 | NONE | | | MTH_698 | unknown | 2.7E−10 |
| MSM0329 | Msp_0265 | conserved hypothetical protein | 7.4E−51 | MTH_700 | conserved protein | 5.1E−64 |
| MSM0330 | Msp_0266 | predicted acyl-CoA synthetase | 1.1E−184 | MTH_701 | acetyl-CoA synthetase | 1.0E−138 |
| | | | | | related protein | |
| MSM0331 | Msp_1390 | KorD | 7.0E−07 | NONE | 2-oxoisovalerate | 7.9E−20 |
| | | | | | oxidoreductase, gamma | |
| | | | | | subunit | |
| MSM0332 | Msp_1389 | KorA | 1.6E−56 | NONE | 2-oxoisovalerate | 6.4E−144 |
| | | | | | oxidoreductase, beta | |
| | | | | | subunit | |
| MSM0333 | Msp_1388 | KorB | 2.0E−28 | NONE | 2-oxoisovalerate | 8.0E−169 |
| | | | | | oxidoreductase, alpha | |
| | | | | | subunit | |
| MSM0334 | Msp_1411 | GatD | 9.1E−140 | MTH_706 | L-asparaginase I | 6.4E−144 |
| MSM0335 | Msp_1412 | GatE | 8.1E−187 | MTH_707 | PET112-like protein | 7.1E−209 |
| MSM0336 | NONE | | | NONE | | |
| MSM0337 | Msp_0145 | member of asn/thr-rich large | 1.1E−08 | NONE | | |
| | | protein family | | | | |
| MSM0338 | NONE | | | NONE | | |
| MSM0339 | NONE | | | NONE | | |
| MSM0340 | Msp_1413 | predicted thioredoxin reductase | 1.4E−70 | MTH_708 | thioredoxin reductase | 6.9E−92 |
| MSM0341 | NONE | | | NONE | | |
| MSM0342 | Msp_0017 | conserved hypothetical protein | 1.7E−28 | NONE | | |
| MSM0343 | Msp_1311 | GMP synthase [glutamine | 4.2E−64 | NONE | GMP synthetase, subunit A | 1.1E−68 |
| | | hydrolyzing], subunit A | | | | |
| MSM0344 | NONE | | | NONE | | |
| MSM0345 | Msp_1312 | GMP synthase [glutamine | 3.4E−117 | NONE | GMP synthetase, subunit B | 7.1E−122 |
| | | hydrolyzing], subunit B | | | | |
| MSM0346 | Msp_1315 | conserved hypothetical protein | 8.0E−125 | MTH_720 | unknown | 3.1E−128 |
| MSM0347 | Msp_1316 | conserved hypothetical protein | 6.5E−43 | MTH_721 | conserved protein | 8.6E−62 |
| MSM0348 | Msp_1317 | conserved hypothetical protein | 7.1E−14 | MTH_722 | conserved protein | 2.3E−22 |
| MSM0349 | Msp_1317 | conserved hypothetical protein | 1.5E−05 | MTH_722 | conserved protein | 1.2E−04 |
| MSM0350 | Msp_1318 | predicted | 3.9E−155 | MTH_723 | 2-isopropylmalate | 6.2E−162 |
| | | isopropylmalate/homocitrate/citramalate | | | synthase | |
| | | synthase | | | | |
| MSM0351 | NONE | | | NONE | | |
| MSM0352 | Msp_1319 | predicted DNA modification | 1.4E−72 | MTH_724 | methyltransferase related | 4.3E−83 |
| | | methylase | | | protein | |
| MSM0353 | Msp_1321 | hypothetical membrane-spanning | 4.8E−11 | NONE | | |
| | | protein | | | | |
| MSM0354 | Msp_1206 | proteasome-activating | 4.1E−144 | MTH_728 | ATP-dependent 26S | 1.2E−172 |
| | | nucleotidase | | | protease regulatory | |
| | | | | | subunit 4 | |
| MSM0355 | Msp_1207 | predicted transcriptional regulator | 7.4E−35 | MTH_729 | conserved protein | 2.7E−33 |
| MSM0356 | Msp_1208 | conserved hypothetical protein | 2.3E−24 | MTH_730 | conserved protein | 6.2E−27 |
| MSM0357 | Msp_1209 | conserved hypothetical | 1.6E−128 | MTH_731 | unknown | 1.5E−110 |
| | | membrane-spanning protein | | | | |
| MSM0358 | Msp_1210 | conserved hypothetical | 7.3E−44 | MTH_733 | unknown | 3.7E−45 |
| | | membrane-spanning protein | | | | |
| MSM0359 | Msp_1213 | predicted UDP-N-acetylmuramyl | 1.7E−108 | MTH_530 | UDP-N-acetylmuramyl | 5.2E−14 |
| | | tripeptide synthase | | | tripeptide synthetase | |
| | | | | | related protein | |
| MSM0360 | Msp_1214 | predicted UDP-N-acetylmuramyl | 1.9E−91 | MTH_735 | phospho-N- | 2.8E−102 |
| | | pentapeptide phosphotransferase | | | acetylmuramoyl- | |
| | | | | | pentapeptide-transferase | |
| MSM0361 | Msp_1215 | partially conserved hypothetical | 6.8E−96 | MTH_736 | conserved protein | 2.0E−76 |
| | | protein, predicted carbamoyl- | | | | |
| | | phosphate synthase, large chain | | | | |
| MSM0362 | Msp_1216 | partially conserved hypothetical | 5.4E−16 | NONE | coenzyme F420-reducing | 5.3E−30 |
| | | protein | | | hydrogenase, delta | |
| | | | | | subunit homolog | |
| MSM0363 | Msp_1217 | predicted RNA methylase | 3.2E−50 | MTH_738 | conserved protein | 1.0E−56 |
| MSM0364 | Msp_1218 | putative nickel responsive | 3.0E−54 | MTH_739 | conserved protein | 9.1E−58 |
| | | regulator | | | | |
| MSM0365 | Msp_1090 | hypothetical protein | 2.1E−23 | MTH_741 | unknown | 1.8E−22 |
| MSM0366 | NONE | | | NONE | | |
| MSM0367 | Msp_0099 | conserved hypothetical protein | 6.0E−17 | MTH_812 | conserved protein | 5.6E−26 |
| MSM0368 | Msp_0667 | putative glutamate synthase, | 1.3E−193 | NONE | glutamate synthase | 1.3E−216 |
| | | subunit 2 with ferredoxin domain | | | (NADPH), alpha subunit | |
| MSM0369 | Msp_0669 | putative glutamate synthase, | 1.2E−68 | NONE | tungsten | 1.1E−82 |
| | | subunit 3 | | | formylmethanofuran | |
| | | | | | dehydrogenase, subunit C | |
| | | | | | homolog | |
| MSM0370 | Msp_0670 | putative glutamate synthase, | 5.7E−115 | MTH_191 | glutamine PRPP | 2.2E−127 |
| | | subunit 1 | | | amidotransferase | |
| MSM0371 | Msp_0671 | predicted glutamine | 6.2E−54 | MTH_190 | conserved protein | 3.3E−60 |
| | | amidotransferase | | | | |
| MSM0372 | Msp_0673 | partially conserved hypothetical | 1.3E−23 | MTH_187 | conserved protein | 2.8E−24 |
| | | protein | | | | |
| MSM0373 | Msp_1484 | LeuB | 3.3E−96 | MTH_184 | isocitrate dehydrogenase | 4.5E−104 |
| MSM0374 | Msp_0447 | predicted acyl-CoA synthetase | 8.3E−178 | MTH_657 | long-chain-fatty-acid-CoA | 5.0E−58 |
| | | | | | ligase | |
| MSM0375 | Msp_0550 | ArgB | 2.3E−111 | MTH_183 | acetylglutamate kinase | 2.5E−110 |
| MSM0376 | Msp_0967 | putative NADP-dependent alcohol | 6.2E−06 | NONE | | |
| | | dehydrogenase | | | | |
| MSM0377 | Msp_0310 | predicted | 4.9E−07 | MTH_1152 | conserved protein | 6.5E−05 |
| | | GTP:adenosylcobinamide- | | | | |
| | | phosphate guanylyltransferase | | | | |
| MSM0378 | NONE | | | MTH_1876 | conserved protein | 1.3E−24 |
| MSM0379 | Msp_0549 | ArgJ | 6.5E−107 | MTH_182 | glutamate N- | 1.9E−103 |
| | | | | | acetyltransferase | |
| MSM0380 | Msp_0506 | hypothetical membrane-spanning | 2.1E−05 | MTH_181 | unknown | 1.8E−04 |
| | | protein | | | | |
| MSM0381 | Msp_0546 | conserved hypothetical | 2.8E−99 | MTH_180 | unknown | 1.4E−114 |
| | | membrane-spanning protein | | | | |
| MSM0382 | Msp_0545 | conserved hypothetical protein | 3.7E−95 | MTH_179 | unknown | 1.9E−103 |
| MSM0383 | Msp_0544 | predicted phosphohydrolase | 1.0E−62 | MTH_178 | lcc related protein | 2.6E−53 |
| MSM0384 | Msp_0543 | conserved hypothetical protein | 4.1E−34 | MTH_177 | conserved protein | 1.9E−34 |
| MSM0385 | Msp_0511 | predicted Fe—S oxidoreductase | 3.2E−07 | MTH_1784 | Mg-protoporphyrin IX | 9.9E−84 |
| | | | | | monomethyl ester | |
| | | | | | oxidative cyclase | |
| MSM0386 | Msp_0148 | predicted sodium:solute | 1.9E−178 | MTH_1856 | sodium/proline symporter | 1.5E−181 |
| | | symporter | | | (proline permease) | |
| MSM0387 | Msp_1040 | coenzyme F390 synthetase II | 2.2E−145 | MTH_1855 | coenzyme F390 | 1.4E−162 |
| | | | | | synthetase II | |
| MSM0388 | Msp_1041 | predicted regulatory protein | 4.1E−34 | MTH_1854 | unknown | 2.6E−37 |
| MSM0389 | Msp_0136 | hypothetical protein | 1.5E−06 | NONE | | |
| MSM0390 | NONE | | | NONE | | |
| MSM0391 | Msp_1042 | IorB | 5.6E−53 | NONE | indolepyruvate | 2.4E−50 |
| | | | | | oxidoreductase, beta | |
| | | | | | subunit | |
| MSM0392 | Msp_1043 | IorA | 6.7E−185 | NONE | indolepyruvate | 4.1E−192 |
| | | | | | oxidoreductase, alpha | |
| | | | | | subunit | |
| MSM0393 | Msp_1044 | TfrB | 3.3E−135 | MTH_1850 | fumarate reductase | 1.4E−155 |
| MSM0394 | Msp_1047 | predicted rRNA methylase | 2.2E−65 | MTH_1849 | conserved protein | 1.2E−69 |
| MSM0395 | Msp_1581 | partially conserved hypothetical | 2.7E−46 | MTH_745 | unknown (contains | 3.9E−57 |
| | | protein | | | ferredoxin domain) | |
| MSM0396 | Msp_0233 | conserved hypothetical protein | 2.3E−22 | NONE | | |
| MSM0397 | NONE | | | NONE | | |
| MSM0398 | Msp_1229 | ribose-phosphate | 6.6E−04 | MTH_1114 | uracil | 6.6E−23 |
| | | pyrophosphokinase | | | phosphoribosyltransferase | |
| MSM0399 | NONE | | | NONE | | |
| MSM0400 | NONE | | | NONE | | |
| MSM0401 | NONE | | | MTH_75 | surface protease related | 2.7E−27 |
| | | | | | protein | |
| MSM0402 | Msp_1048 | deoxycytidine triphosphate | 3.5E−76 | MTH_1847 | deoxycytidine | 1.1E−75 |
| | | deaminase | | | triphosphate deaminase | |
| MSM0403 | Msp_1049 | GlyS | 2.1E−188 | MTH_1846 | glycyl-tRNA synthetase | 7.6E−196 |
| MSM0404 | Msp_0799 | predicted transcriptional regulator | 1.6E−25 | MTH_1843 | unknown | 9.1E−26 |
| MSM0405 | Msp_1050 | predicted metal-dependent | 1.7E−58 | MTH_1842 | conserved protein | 2.5E−46 |
| | | hydrolase | | | | |
| MSM0406 | Msp_1052 | hypothetical protein | 1.7E−10 | MTH_1838 | unknown | 6.6E−23 |
| MSM0407 | Msp_1053 | conserved hypothetical | 1.7E−115 | MTH_1837 | unknown | 1.2E−124 |
| | | membrane-spanning protein | | | | |
| MSM0408 | Msp_0406 | 2-phosphoglycerate kinase- | 4.2E−80 | MTH_1835 | 2-phosphoglycerate | 2.3E−91 |
| | | like/predicted small molecule- | | | kinase homolog | |
| | | binding domain fusion | | | | |
| MSM0409 | Msp_0407 | conserved hypothetical protein | 2.2E−42 | MTH_1834 | conserved protein | 9.5E−47 |
| MSM0410 | Msp_0409 | conserved hypothetical protein | 3.9E−52 | MTH_1833 | unknown | 4.6E−47 |
| MSM0411 | Msp_0145 | member of asn/thr-rich large | 1.3E−25 | MTH_1074 | putative membrane | 1.3E−115 |
| | | protein family | | | protein | |
| MSM0412 | Msp_0046 | member of asn/thr-rich large | 1.3E−06 | MTH_117 | unknown | 2.4E−41 |
| | | protein family | | | | |
| MSM0413 | Msp_0512 | predicted transcriptional regulator | 2.7E−21 | MTH_313 | transcriptional regulator | 1.9E−16 |
| MSM0414 | Msp_0824 | predicted Na+-driven multidrug | 2.8E−138 | MTH_314 | conserved protein | 6.7E−110 |
| | | efflux pump | | | | |
| MSM0415 | Msp_1362 | PyrH | 3.5E−76 | MTH_879 | uridine monophosphate | 2.8E−79 |
| | | | | | kinase | |
| MSM0416 | Msp_0974 | predicted Mg-dependent DNase | 1.5E−93 | MTH_233 | conserved protein | 8.0E−27 |
| MSM0417 | Msp_1361 | hypothetical membrane-spanning | 3.8E−15 | MTH_880 | unknown | 3.2E−14 |
| | | protein | | | | |
| MSM0418 | Msp_1045 | conserved hypothetical protein | 2.5E−34 | MTH_507 | conserved protein | 2.5E−32 |
| MSM0419 | Msp_0253 | conserved hypothetical | 1.4E−24 | MTH_506 | unknown | 4.2E−21 |
| | | membrane-spanning protein | | | | |
| MSM0420 | Msp_0355 | conserved hypothetical | 3.0E−22 | MTH_882 | conserved protein | 1.1E−27 |
| | | membrane-spanning protein | | | | |
| MSM0421 | NONE | | | NONE | | |
| MSM0422 | Msp_0644 | conserved hypothetical | 1.1E−36 | MTH_883 | unknown | 6.3E−48 |
| | | membrane-spanning protein | | | | |
| MSM0423 | Msp_0645 | predicted glycosyltransferase | 6.9E−157 | MTH_884 | teichoic acid biosynthesis | 4.5E−184 |
| | | | | | related protein | |
| MSM0424 | Msp_1360 | transcription initiation factor IIB | 8.1E−148 | MTH_885 | transcription initiation | 9.2E−152 |
| | | (TFIIB) | | | factor TFIIB | |
| MSM0425 | Msp_1359 | hypothetical protein | 2.3E−15 | MTH_886 | conserved protein | 3.4E−19 |
| MSM0426 | Msp_1358 | predicted demethylmenaquinone | 3.7E−33 | MTH_888 | conserved protein | 3.2E−46 |
| | | methyltransferase | | | | |
| MSM0427 | Msp_1356 | predicted DNA primase | 7.2E−108 | MTH_891 | conserved protein | 2.9E−141 |
| MSM0428 | Msp_1355 | predicted site-specific | 2.5E−66 | MTH_893 | integrase-recombinase | 7.7E−77 |
| | | recombinase/integrase | | | protein | |
| MSM0429 | Msp_1354 | conserved hypothetical protein | 4.3E−46 | MTH_905 | conserved protein | 1.8E−38 |
| MSM0430 | NONE | | | MTH_906 | unknown | 2.7E−17 |
| MSM0431 | Msp_1132 | predicted ATP-dependent | 1.7E−44 | MTH_947 | conserved protein | 2.8E−40 |
| | | carboligase | | | | |
| MSM0432 | Msp_1131 | hypothetical membrane-spanning | 5.5E−07 | NONE | | |
| | | protein | | | | |
| MSM0433 | Msp_1133 | AhaD | 1.6E−69 | NONE | ATP synthase, subunit D | 1.5E−73 |
| MSM0434 | Msp_1134 | AhaB | 1.4E−212 | NONE | ATP synthase, subunit B | 4.5E−214 |
| MSM0435 | Msp_1135 | AhaA | 1.4E−246 | NONE | ATP synthase, subunit A | 2.8E−260 |
| MSM0436 | Msp_1136 | AhaF | 8.6E−25 | NONE | ATP synthase, subunit F | 3.1E−25 |
| MSM0437 | Msp_1137 | AhaC | 1.5E−105 | NONE | ATP synthase, subunit C | 7.7E−116 |
| MSM0438 | Msp_1138 | AhaE | 3.2E−50 | NONE | ATP synthase, subunit E | 5.9E−54 |
| MSM0439 | Msp_1139 | AhaK | 7.0E−62 | NONE | ATP synthase, subunit K | 9.7E−70 |
| MSM0440 | Msp_1140 | AhaI | 1.9E−148 | NONE | ATP synthase, subunit I | 3.5E−191 |
| MSM0441 | Msp_1141 | AhaH | 7.6E−17 | MTH_961 | unknown | 3.1E−18 |
| MSM0442 | NONE | | | NONE | | |
| MSM0443 | NONE | | | NONE | | |
| MSM0444 | NONE | | | NONE | | |
| MSM0445 | Msp_0408 | putative nitroreductase protein | 2.0E−55 | MTH_120 | NADPH-oxidoreductase | 1.4E−13 |
| MSM0446 | NONE | | | MTH_962 | citrate synthase I | 6.2E−75 |
| MSM0447 | Msp_0338 | fumarate hydratase | 2.6E−15 | NONE | fumarate hydratase, class | 3.8E−75 |
| | | | | | I related protein | |
| MSM0448 | NONE | | | MTH_964 | unknown | 4.6E−102 |
| MSM0449 | NONE | | | MTH_965 | conserved protein | 1.1E−86 |
| MSM0450 | Msp_0680 | conserved hypothetical | 2.4E−38 | NONE | | |
| | | membrane-spanning protein | | | | |
| MSM0451 | Msp_0679 | conserved hypothetical | 7.8E−79 | NONE | | |
| | | membrane-spanning protein | | | | |
| MSM0452 | Msp_1142 | predicted DNA-binding protein | 3.9E−132 | MTH_966 | conserved protein | 1.8E−130 |
| MSM0453 | Msp_1143 | putative transcriptional regulator | 7.5E−58 | MTH_967 | conserved protein | 1.3E−88 |
| MSM0454 | NONE | | | NONE | | |
| MSM0455 | Msp_1144 | conserved hypothetical protein | 2.2E−35 | MTH_969 | unknown | 1.0E−43 |
| MSM0456 | Msp_1005 | conserved hypothetical protein | 2.3E−17 | MTH_544 | conserved protein | 2.7E−35 |
| MSM0457 | Msp_1145 | SerA | 8.8E−158 | MTH_970 | phosphoglycerate | 1.3E−177 |
| | | | | | dehydrogenase | |
| MSM0458 | NONE | | | NONE | | |
| MSM0459 | NONE | | | NONE | | |
| MSM0460 | NONE | | | NONE | | |
| MSM0461 | Msp_0983 | member of asn/thr-rich large | 3.0E−39 | MTH_911 | probable surface protein | 2.9E−18 |
| | | protein family | | | | |
| MSM0462 | Msp_1146 | partially conserved hypothetical | 1.8E−38 | MTH_971 | unknown | 1.0E−33 |
| | | protein | | | | |
| MSM0463 | Msp_1147 | conserved hypothetical protein | 2.0E−57 | MTH_972 | conserved protein | 3.7E−61 |
| MSM0464 | Msp_1148 | predicted dinucleotide-utilizing | 4.0E−59 | MTH_973 | conserved protein | 1.1E−77 |
| | | protein | | | | |
| MSM0465 | Msp_1149 | conserved hypothetical protein | 1.1E−17 | MTH_974 | unknown | 4.1E−23 |
| MSM0466 | Msp_1150 | predicted tRNA-binding protein | 2.4E−68 | MTH_975 | conserved protein | 1.4E−70 |
| MSM0467 | NONE | | | MTH_978 | NADP-dependent | 8.1E−137 |
| | | | | | glyceraldehyde-3- | |
| | | | | | phosphate | |
| | | | | | dehydrogenase | |
| MSM0468 | NONE | | | MTH_1490 | unknown | 2.2E−10 |
| MSM0469 | NONE | | | MTH_1490 | unknown | 1.8E−11 |
| MSM0470 | Msp_1151 | hypothetical membrane-spanning | 1.4E−10 | MTH_979 | unknown | 7.2E−10 |
| | | protein | | | | |
| MSM0471 | Msp_1152 | conserved hypothetical | 7.1E−53 | MTH_980 | conserved protein | 5.9E−70 |
| | | membrane-spanning protein | | | | |
| MSM0472 | Msp_1153 | PepQ | 2.7E−69 | MTH_981 | aminopeptidase P | 1.0E−65 |
| MSM0473 | Msp_0417 | hypothetical membrane-spanning | 2.5E−04 | NONE | | |
| | | protein | | | | |
| MSM0474 | NONE | | | NONE | | |
| MSM0475 | Msp_0417 | hypothetical membrane-spanning | 1.8E−04 | NONE | | |
| | | protein | | | | |
| MSM0476 | NONE | | | MTH_93 | unknown | 8.5E−04 |
| MSM0477 | NONE | | | NONE | | |
| MSM0478 | NONE | | | NONE | | |
| MSM0479 | Msp_1154 | conserved hypothetical | 2.4E−45 | MTH_986 | conserved protein | 2.1E−42 |
| | | membrane-spanning protein | | | | |
| MSM0480 | Msp_1155 | conserved hypothetical protein | 2.3E−95 | MTH_987 | conserved protein | 6.0E−109 |
| MSM0481 | Msp_1274 | conserved hypothetical protein | 4.4E−53 | MTH_989 | conserved protein | 2.2E−24 |
| MSM0482 | Msp_1275 | predicted ATP-utilizing enzyme | 4.6E−58 | MTH_990 | conserved protein | 2.6E−51 |
| MSM0483 | NONE | | | MTH_991 | unknown | 8.6E−14 |
| MSM0484 | Msp_1276 | conserved hypothetical protein | 9.2E−76 | MTH_992 | inosine-5′- | 2.8E−86 |
| | | | | | monophosphate | |
| | | | | | dehydrogenase related | |
| | | | | | protein IX | |
| MSM0485 | Msp_1410 | predicted universal stress protein | 9.6E−26 | MTH_993 | conserved protein | 1.0E−33 |
| MSM0486 | Msp_1199 | predicted metal-dependent | 3.1E−84 | MTH_994 | N-ethylammeline | 4.2E−85 |
| | | hydrolase | | | chlorohydrolase related | |
| | | | | | protein | |
| MSM0487 | NONE | | | NONE | | |
| MSM0488 | Msp_1200 | CarB | 0.0E+00 | NONE | carbamoyl-phosphate | 0.0E+00 |
| | | | | | synthase, large subunit | |
| MSM0489 | Msp_1201 | CarA | 1.5E−121 | NONE | carbamoyl-phosphate | 6.0E−125 |
| | | | | | synthase, small subunit | |
| MSM0490 | Msp_0602 | conserved hypothetical protein | 1.0E−28 | MTH_738 | conserved protein | 3.0E−06 |
| MSM0491 | Msp_0410 | NadC | 2.0E−64 | MTH_1832 | quinolinate | 7.7E−61 |
| | | | | | phosphoribosyltransferase | |
| MSM0492 | Msp_0411 | putative ribonuclease Z | 1.7E−76 | MTH_1831 | conserved protein | 2.6E−92 |
| MSM0493 | Msp_0982 | predicted mechanosensitive ion | 6.7E−25 | MTH_1830 | conserved protein | 1.7E−40 |
| | | channel | | | | |
| MSM0494 | Msp_0643 | NadA | 3.6E−90 | MTH_1827 | quinolinate synthetase | 6.8E−101 |
| MSM0495 | NONE | | | MTH_1821 | unknown | 2.7E−19 |
| MSM0496 | Msp_1526 | putative homoserine O- | 1.2E−84 | MTH_1820 | homoserine O- | 1.3E−67 |
| | | acetyltransferase | | | acetyltransferase | |
| MSM0497 | Msp_0157 | hypothetical protein | 6.9E−55 | MTH_1816 | conserved protein | 2.6E−76 |
| MSM0498 | NONE | | | NONE | | |
| MSM0499 | Msp_1548 | hypothetical protein | 1.0E−05 | MTH_1277 | unknown | 1.8E−06 |
| MSM0500 | Msp_0155 | predicted amidohydrolase | 3.1E−75 | MTH_1811 | N-carbamoyl-D-amino | 3.7E−77 |
| | | | | | acid amidohydrolase | |
| MSM0501 | Msp_0153 | conserved hypothetical protein | 1.8E−31 | MTH_1806 | phycocyanin alpha | 8.1E−34 |
| | | | | | phycocyanobilin lyase | |
| | | | | | CpcE | |
| MSM0502 | Msp_0150 | predicted helicase | 2.9e−310 | MTH_1802 | ATP-dependent helicase | 0.0E+00 |
| MSM0503 | Msp_0553 | hypothetical protein | 9.4E−19 | MTH_1799 | unknown | 3.9E−18 |
| MSM0504 | Msp_0927 | hypothetical protein | 2.1E−05 | MTH_1641 | unknown | 1.4E−06 |
| MSM0505 | NONE | | | NONE | | |
| MSM0506 | Msp_0240 | predicted ATP-utilizing enzyme | 3.0E−148 | MTH_1201 | conserved protein | 3.4E−145 |
| MSM0507 | Msp_0365 | predicted phosphoesterase | 6.0E−49 | MTH_1774 | conserved protein | 2.9E−52 |
| MSM0508 | Msp_0364 | putative 23S rRNA methylase | 1.9E−61 | MTH_1773 | cell division protein J | 5.9E−70 |
| MSM0509 | Msp_0363 | hypothetical membrane-spanning | 1.4E−24 | MTH_1772 | unknown | 9.1E−26 |
| | | protein | | | | |
| MSM0510 | Msp_0362 | predicted minichromosome | 1.4E−255 | MTH_1770 | DNA replication initiator | 1.4E−260 |
| | | maintenance protein | | | (Cdc21/Cdc54) | |
| MSM0511 | Msp_0361 | translation initiation factor aIF-2, | 2.3E−54 | NONE | translation initiation factor | 6.9E−60 |
| | | beta subunit (eIF2B) | | | eIF-2, beta subunit | |
| MSM0512 | Msp_0360 | predicted NMD3-related protein | 5.2E−73 | MTH_1768 | conserved protein | 2.1E−90 |
| MSM0513 | Msp_0359 | TyrS | 2.4E−100 | MTH_1767 | tyrosyl-tRNA synthetase | 1.1E−109 |
| MSM0514 | Msp_0358 | hypothetical protein | 3.5E−05 | MTH_1766 | unknown | 1.1E−08 |
| MSM0515 | Msp_0186 | MtaB2 | 1.3E−156 | NONE | | |
| MSM0516 | Msp_0185 | MtaC3 | 5.2E−89 | NONE | | |
| MSM0517 | Msp_0190 | MapA | 8.7E−167 | MTH_278 | ferredoxin | 7.0E−04 |
| MSM0518 | Msp_0112 | MtaA2 | 2.1E−94 | MTH_775 | cobalamin-independent | 3.4E−05 |
| | | | | | methionine synthase | |
| MSM0519 | Msp_0183 | hypothetical protein | 1.2E−32 | NONE | | |
| MSM0520 | Msp_0357 | putative thymidylate kinase | 2.1E−46 | MTH_1765 | thymidylate kinase | 7.5E−47 |
| MSM0521 | NONE | | | NONE | | |
| MSM0522 | Msp_0984 | predicted peptidase | 2.7E−234 | MTH_1763 | collagenase | 3.4E−99 |
| MSM0523 | Msp_0984 | predicted peptidase | 1.6E−96 | MTH_1763 | collagenase | 6.8E−108 |
| MSM0524 | Msp_0354 | MutS | 4.3E−133 | MTH_1762 | DNA mismatch | 1.9E−176 |
| | | | | | recognition protein MutS | |
| MSM0525 | Msp_1282 | predicted protein kinase | 1.8E−104 | MTH_1645 | ABC transporter | 3.1E−112 |
| MSM0526 | NONE | | | NONE | | |
| MSM0527 | Msp_0017 | conserved hypothetical protein | 3.5E−28 | NONE | | |
| MSM0528 | Msp_0233 | conserved hypothetical protein | 1.4E−10 | NONE | | |
| MSM0529 | Msp_0725 | hypothetical protein | 1.0E−04 | NONE | | |
| MSM0530 | Msp_1323 | conserved hypothetical protein | 3.3E−04 | MTH_72 | O-linked GlcNAc | 5.5E−06 |
| | | | | | transferase | |
| MSM0531 | NONE | | | NONE | | |
| MSM0532 | Msp_0233 | conserved hypothetical protein | 3.4E−08 | NONE | | |
| MSM0533 | Msp_0017 | conserved hypothetical protein | 3.1E−16 | NONE | | |
| MSM0534 | NONE | | | NONE | | |
| MSM0535 | Msp_0466 | hypothetical protein | 7.1E−05 | NONE | | |
| MSM0536 | NONE | | | NONE | | |
| MSM0537 | NONE | | | NONE | | |
| MSM0538 | Msp_1324 | predicted glycyl radical activating | 5.1E−07 | MTH_1586 | pyruvate formate-lyase | 1.3E−05 |
| | | enzyme | | | activating enzyme | |
| MSM0539 | Msp_0219 | conserved hypothetical protein | 3.1E−04 | NONE | | |
| MSM0540 | NONE | | | NONE | | |
| MSM0541 | NONE | | | NONE | | |
| MSM0542 | Msp_1128 | F420-dependent N5,N10- | 3.4E−94 | NONE | coenzyme F420- | 1.4E−132 |
| | | methylenetetrahydromethanopterin | | | dependent N5,N10- | |
| | | reductase | | | methylene | |
| | | | | | tetrahydromethanopterin | |
| | | | | | reductase | |
| MSM0543 | Msp_0646 | predicted DNA repair photolyase | 9.3E−28 | NONE | | |
| MSM0544 | Msp_1127 | predicted Fe—S oxidoreductase | 4.4E−92 | MTH_1751 | conserved protein | 1.3E−90 |
| MSM0545 | NONE | | | NONE | | |
| MSM0546 | Msp_1046 | hypothetical membrane-spanning | 2.6E−23 | MTH_813 | unknown | 2.4E−27 |
| | | protein | | | | |
| MSM0547 | Msp_0324 | predicted nucleotidyltransferase | 1.6E−08 | MTH_1749 | unknown | 7.2E−81 |
| MSM0548 | Msp_1148 | predicted dinucleotide-utilizing | 4.4E−04 | MTH_1747 | conserved protein | 5.4E−37 |
| | | protein | | | | |
| MSM0549 | Msp_0830 | Trk-type potassium transport | 3.9E−04 | MTH_1746 | cytochrome C-type | 2.1E−28 |
| | | system, membrane protein | | | biogenesis protein | |
| MSM0550 | Msp_0656 | hypothetical membrane-spanning | 2.0E−04 | MTH_1745 | protein disulphide | 7.9E−20 |
| | | protein | | | isomerase | |
| MSM0551 | Msp_1124 | conserved hypothetical protein | 1.9E−68 | MTH_1744 | conserved protein | 2.4E−73 |
| MSM0552 | Msp_0330 | hypothetical protein | 4.6E−10 | MTH_1743 | unknown | 8.9E−12 |
| MSM0553 | Msp_0331 | predicted ATPase | 3.5E−92 | MTH_1742 | conserved protein | 1.2E−80 |
| MSM0554 | Msp_0161 | conserved hypothetical protein | 2.8E−74 | MTH_1815 | conserved protein | 2.6E−83 |
| MSM0555 | Msp_0192 | predicted MoxR-like ATPase | 3.9E−93 | MTH_1814 | conserved protein | 1.9E−87 |
| MSM0556 | Msp_0333 | predicted pterin-binding enzyme | 4.1E−121 | MTH_1741 | conserved protein | 1.1E−153 |
| MSM0557 | Msp_0334 | PorC | 2.1E−53 | NONE | pyruvate oxidoreductase, | 2.1E−65 |
| | | | | | gamma subunit | |
| MSM0558 | Msp_0335 | PorD | 4.3E−30 | NONE | pyruvate oxidoreductase, | 1.2E−32 |
| | | | | | gamma subunit | |
| MSM0559 | Msp_0336 | PorA | 2.1E−140 | NONE | pyruvate oxidoreductase, | 2.3E−148 |
| | | | | | alpha subunit | |
| MSM0560 | Msp_0337 | PorB | 1.8E−118 | NONE | pyruvate oxidoreductase, | 2.2E−127 |
| | | | | | beta subunit | |
| MSM0561 | Msp_1447 | EhbK | 8.6E−08 | NONE | formate hydrogenlyase, | 4.5E−40 |
| | | | | | iron-sulfur subunit I | |
| MSM0562 | Msp_1447 | EhbK | 4.0E−09 | NONE | formate hydrogenlyase, | 5.3E−14 |
| | | | | | iron-sulfur subunit 2 | |
| MSM0563 | Msp_0338 | fumarate hydratase | 3.3E−96 | NONE | fumarate hydratase, class I | 8.3E−96 |
| MSM0564 | Msp_0339 | predicted phosphate uptake | 4.8E−31 | MTH_1734 | phosphate transport | 2.8E−47 |
| | | regulator | | | system regulator | |
| MSM0565 | Msp_0340 | PstB | 4.0E−107 | MTH_1731 | phosphate transport | 1.5E−105 |
| | | | | | system ATP-binding | |
| MSM0566 | Msp_0341 | PstA | 1.3E−94 | MTH_1730 | phosphate transporter | 4.5E−111 |
| | | | | | permease PstC homolog | |
| MSM0567 | Msp_0342 | PstC | 7.0E−94 | MTH_1729 | phosphate transporter | 4.8E−100 |
| | | | | | permease PstC | |
| MSM0568 | Msp_0343 | PstS | 1.6E−64 | MTH_1727 | phosphate-binding protein | 2.7E−81 |
| | | | | | PstS | |
| MSM0569 | Msp_0344 | predicted phosphate uptake | 5.5E−62 | MTH_1724 | phosphate transport | 2.4E−82 |
| | | regulator | | | system regulator related | |
| | | | | | protein | |
| MSM0570 | Msp_0346 | conserved hypothetical | 5.2E−17 | MTH_1723 | unknown | 9.1E−26 |
| | | membrane-spanning protein | | | | |
| MSM0571 | NONE | | | MTH_1137 | conserved protein (FlpA) | 5.2E−165 |
| MSM0572 | NONE | | | NONE | H(2)-dependent N5,N10- | 2.4E−128 |
| | | | | | methylenetetrahydromethanopterin | |
| | | | | | dehydrogenase | |
| MSM0573 | Msp_0296 | CofG | 1.4E−15 | MTH_1143 | biotin synthetase (BioB) | 5.1E−112 |
| MSM0574 | NONE | | | MTH_1144 | conserved protein | 2.9E−38 |
| MSM0575 | Msp_1393 | conserved hypothetical | 8.5E−05 | MTH_1145 | conserved protein | 2.9E−38 |
| | | membrane-spanning protein | | | | |
| MSM0576 | NONE | | | MTH_1146 | conserved protein | 2.9E−38 |
| MSM0577 | NONE | | | MTH_1147 | conserved protein | 6.1E−52 |
| MSM0578 | NONE | | | MTH_1148 | conserved protein | 8.1E−34 |
| MSM0579 | Msp_1581 | partially conserved hypothetical | 7.5E−10 | MTH_1106 | ferredoxin | 1.3E−10 |
| | | protein | | | | |
| MSM0580 | Msp_0911 | member of asn/thr-rich large | 2.5E−05 | MTH_654 | unknown | 5.2E−39 |
| | | protein family | | | | |
| MSM0581 | Msp_0166 | conserved hypothetical | 3.9E−29 | MTH_655 | conserved protein | 6.7E−94 |
| | | membrane-spanning protein | | | | |
| MSM0582 | Msp_0737 | putative peptide methionine | 4.5E−122 | MTH_535 | peptide methionine | 2.4E−34 |
| | | sulfoxide reductase MsrA/MsrB | | | sulfoxide reductase | |
| MSM0583 | Msp_0655 | CbiM2 | 2.7E−69 | MTH_1707 | cobalamin biosynthesis | 1.5E−64 |
| | | | | | protein M | |
| MSM0584 | Msp_0656 | hypothetical membrane-spanning | 2.2E−12 | MTH_1706 | unknown | 3.4E−12 |
| | | protein | | | | |
| MSM0585 | Msp_0657 | CbiQ2 | 5.4E−55 | MTH_1705 | cobalt transport | 4.2E−60 |
| | | | | | membrane protein | |
| MSM0586 | Msp_0401 | CbiO1 | 7.6E−81 | MTH_1704 | cobalt transport ATP- | 1.2E−85 |
| | | | | | binding protein O | |
| MSM0587 | Msp_1438 | hypothetical protein | 5.9E−10 | NONE | | |
| MSM0588 | Msp_1441 | FeoA | 1.7E−12 | MTH_1362 | unknown | 2.4E−11 |
| MSM0589 | Msp_1440 | FeoB | 3.6E−200 | MTH_1361 | ferrous iron transport | 5.7E−152 |
| | | | | | protein B | |
| MSM0590 | NONE | | | NONE | | |
| MSM0591 | NONE | | | NONE | | |
| MSM0592 | Msp_0202 | conserved hypothetical | 2.3E−40 | MTH_230 | unknown | 1.2E−48 |
| | | membrane-spanning protein | | | | |
| MSM0593 | Msp_0610 | predicted ABC-type multidrug | 3.9E−77 | MTH_1487 | ABC transporter (ATP- | 2.0E−37 |
| | | transport system, ATP-binding | | | binding | |
| | | protein | | | | |
| MSM0594 | Msp_0609 | conserved hypothetical | 2.7E−44 | NONE | | |
| | | membrane-spanning protein | | | | |
| MSM0595 | Msp_0609 | conserved hypothetical | 1.8E−40 | NONE | | |
| | | membrane-spanning protein | | | | |
| MSM0596 | Msp_1163 | predicted type II secretion protein F | 3.0E−47 | MTH_1703 | unknown | 4.9E−59 |
| MSM0597 | Msp_1162 | predicted type II/IV secretion | 4.1E−121 | MTH_1702 | secretory protein kinase | 2.9E−157 |
| | | protein | | | | |
| MSM0598 | Msp_1161 | conserved hypothetical protein | 3.5E−44 | MTH_1701 | unknown | 5.6E−42 |
| MSM0599 | Msp_1160 | conserved hypothetical | 1.3E−94 | MTH_1700 | conserved protein | 8.9E−99 |
| | | membrane-spanning protein | | | | |
| MSM0600 | Msp_0512 | predicted transcriptional regulator | 7.9E−15 | MTH_313 | transcriptional regulator | 5.5E−12 |
| MSM0601 | Msp_0017 | conserved hypothetical protein | 1.7E−28 | NONE | | |
| MSM0602 | Msp_1159 | elongation factor 1-beta (aEF- | 2.2E−26 | MTH_1699 | translation elongation | 1.3E−28 |
| | | 1beta) (ef1B) | | | factor EF-1b | |
| MSM0603 | Msp_1158 | predicted Zn-ribbon RNA-binding | 4.7E−17 | MTH_1178 | conserved protein | 8.3E−04 |
| | | protein | | | | |
| MSM0604 | Msp_1157 | predicted amino acid kinase | 1.7E−42 | MTH_1698 | delta 1-pyrroline-5- | 6.2E−43 |
| | | | | | carboxylate synthetase | |
| MSM0605 | Msp_1156 | putative peptidyl-tRNA hydrolase | 1.5E−29 | MTH_1697 | conserved protein | 1.1E−36 |
| MSM0606 | NONE | | | NONE | | |
| MSM0607 | Msp_0613 | predicted ATPase | 4.1E−224 | MTH_1695 | RNase L inhibitor | 6.8E−227 |
| MSM0608 | NONE | | | NONE | | |
| MSM0609 | Msp_0147 | ferredoxin | 2.6E−04 | MTH_221 | unknown | 6.4E−25 |
| MSM0610 | Msp_0370 | putative aspartate | 8.5E−121 | MTH_1694 | aspartate | 9.6E−134 |
| | | aminotransferase | | | aminotransferase related | |
| | | | | | protein | |
| MSM0611 | Msp_0369 | RadB | 3.9E−61 | MTH_1693 | DNA repair protein Rad51 | 3.6E−63 |
| | | | | | homolog | |
| MSM0612 | Msp_0096 | conserved hypothetical protein | 1.9E−36 | MTH_1692 | conserved protein | 3.8E−43 |
| MSM0613 | Msp_0095 | predicted | 1.0E−46 | MTH_1691 | conserved protein | 4.3E−44 |
| | | phosphatidylglycerophosphate | | | | |
| | | synthase | | | | |
| MSM0614 | Msp_0094 | conserved hypothetical protein | 2.1E−14 | MTH_1690 | unknown | 1.7E−17 |
| MSM0615 | Msp_0675 | conserved hypothetical protein | 4.7E−159 | MTH_1686 | conserved protein | 7.7E−164 |
| MSM0616 | Msp_0440 | member of asn/thr-rich large | 1.1E−93 | MTH_716 | cell surface glycoprotein | 1.4E−14 |
| | | protein family | | | (s-layer protein) | |
| MSM0617 | Msp_0160 | Thil | 1.4E−102 | MTH_1685 | conserved protein | 1.1E−118 |
| MSM0618 | Msp_1489 | predicted potassium transport | 3.0E−09 | MTH_760 | Na+/H+-exchanging | 2.3E−16 |
| | | system, membrane component | | | protein:Na+/H+ antiporter | |
| MSM0619 | Msp_1262 | AlaS | 7.0E−300 | MTH_1683 | alanyl-tRNA synthetase | 1.5e−316 |
| MSM0620 | Msp_1263 | 50S ribosomal protein L12P | 1.9E−36 | MTH_1682 | ribosomal protein Lp1 | 9.4E−40 |
| MSM0621 | Msp_1264 | 50S ribosomal protein L10P | 5.3E−96 | MTH_1681 | ribosomal protein Lp0 | 2.7E−106 |
| | | | | | (E. coli) | |
| MSM0622 | Msp_1265 | 50S ribosomal protein L1P | 9.5E−74 | MTH_1680 | ribosomal protein L10a | 1.3E−81 |
| | | | | | (E. coli) | |
| MSM0623 | Msp_1266 | 50S ribosomal protein L11P | 1.3E−62 | MTH_1679 | ribosomal protein L12 | 2.2E−63 |
| | | | | | (E. coli) | |
| MSM0624 | Msp_1267 | putative transcription | 1.3E−46 | MTH_1678 | transcription termination | 1.1E−61 |
| | | antiterminator | | | factor NusG | |
| MSM0625 | Msp_1268 | partially conserved hypothetical | 1.3E−12 | MTH_1677 | protein translocation | 1.1E−13 |
| | | membrane-spanning protein | | | complex sec61 gamma | |
| | | | | | subunit related protein | |
| MSM0626 | Msp_1269 | FtsZ | 8.7E−135 | MTH_1676 | cell division protein FtsZ | 1.7E−143 |
| MSM0627 | Msp_0307 | MtrH | 8.5E−105 | MTH_1156 | N5-methyl- | 3.7E−116 |
| | | | | | tetrahydromethanopterin: | |
| | | | | | coenzyme M | |
| | | | | | methyltransferase, | |
| | | | | | subunit H | |
| MSM0628 | NONE | | | MTH_1675 | conserved protein | 7.2E−49 |
| MSM0629 | Msp_0017 | conserved hypothetical protein | 1.7E−28 | NONE | | |
| MSM0630 | Msp_1271 | conserved hypothetical protein | 7.1E−69 | MTH_1670 | conserved protein | 4.2E−76 |
| MSM0631 | Msp_1272 | predicted transcription initiation | 3.4E−37 | MTH_1669 | conserved protein | 4.6E−47 |
| | | factor IIE, alpha subunit | | | | |
| MSM0632 | Msp_1273 | conserved hypothetical protein | 6.2E−38 | MTH_1668 | conserved protein | 1.7E−40 |
| MSM0633 | Msp_1063 | predicted RNA-binding protein | 9.2E−92 | MTH_1665 | conserved protein | 6.9E−92 |
| MSM0634 | Msp_1064 | conserved hypothetical protein | 1.8E−24 | MTH_1664 | conserved protein | 6.2E−27 |
| MSM0635 | Msp_1069 | predicted regulator of aminoacid | 1.6E−41 | MTH_1654 | unknown | 1.8E−45 |
| | | metabolism | | | | |
| MSM0636 | Msp_1067 | hypothetical protein | 1.6E−23 | MTH_1649 | hydrogenase | 1.2E−25 |
| | | | | | expression/formation | |
| | | | | | protein HypC | |
| MSM0637 | Msp_1077 | predicted dihydrolipoamide | 2.4E−93 | MTH_1648 | dihydrolipoamide | 1.2E−92 |
| | | dehydrogenase-related protein | | | dehydrogenase | |
| MSM0638 | Msp_1343 | hypothetical membrane-spanning | 2.6E−78 | MTH_1646 | unknown | 5.9E−54 |
| | | multicopy protein A 3 | | | | |
| MSM0639 | Msp_1080 | conserved hypothetical | 4.5E−67 | MTH_1644 | unknown | 1.8E−52 |
| | | membrane-spanning protein | | | | |
| MSM0640 | Msp_1081 | predicted release factor aRF1 | 2.2E−106 | MTH_1642 | cell division protein | 9.6E−118 |
| MSM0641 | Msp_1083 | putative prephenate | 4.4E−92 | MTH_1640 | chorismate mutase | 1.8E−100 |
| | | dehydrogenase | | | | |
| MSM0642 | Msp_1084 | CdcH | 9.3E−273 | MTH_1639 | cell division control | 4.7E−299 |
| | | | | | protein Cdc48 | |
| MSM0643 | Msp_0227 | conserved hypothetical protein | 3.3E−71 | MTH_1574 | conserved protein | 5.2E−78 |
| MSM0644 | Msp_0228 | ThiC1 | 1.2E−144 | MTH_1576 | thiamine biosynthesis | 3.2E−158 |
| | | | | | protein | |
| MSM0645 | Msp_0258 | ATP-dependent DNA ligase | 1.1E−148 | MTH_1580 | DNA ligase | 3.9E−176 |
| MSM0646 | Msp_0504 | conserved hypothetical | 5.5E−30 | NONE | | |
| | | membrane-spanning protein | | | | |
| MSM0647 | Msp_0259 | hypothetical protein | 3.8E−15 | MTH_1581 | conserved protein | 4.8E−20 |
| MSM0648 | Msp_0263 | predicted phosphomannomutase | 1.2E−169 | MTH_1584 | phosphomannomutase | 9.9E−171 |
| MSM0649 | Msp_0970 | hypothetical membrane-spanning | 3.5E−44 | MTH_559 | conserved protein | 1.0E−06 |
| | | protein | | | | |
| MSM0650 | Msp_0971 | hypothetical protein | 1.2E−36 | MTH_1787 | conserved protein | 1.3E−07 |
| MSM0651 | Msp_1323 | conserved hypothetical protein | 1.5E−98 | MTH_1585 | O-linked GlcNAc | 1.9E−105 |
| | | | | | transferase | |
| MSM0652 | Msp_1324 | predicted glycyl radical activating | 6.3E−45 | MTH_1586 | pyruvate formate-lyase | 1.5E−50 |
| | | enzyme | | | activating enzyme | |
| MSM0653 | Msp_1326 | HisC | 2.5E−112 | MTH_1587 | histidinol-phosphate | 1.2E−119 |
| | | | | | aminotransferase | |
| MSM0654 | Msp_1325 | predicted carbonic | 1.8E−47 | MTH_1588 | ferripyochelin binding | 4.6E−47 |
| | | anhydrase/acetyltransferase | | | protein | |
| MSM0655 | Msp_1301 | predicted nucleoside- | 3.0E−134 | MTH_1589 | glucose-1-phosphate | 8.1E−137 |
| | | diphosphate-sugar | | | thymidylyltransferase | |
| | | pyrophosphorylase | | | homolog | |
| MSM0656 | Msp_1300 | predicted phosphomannomutase | 9.7E−136 | MTH_1590 | phosphomannomutase | 7.6E−141 |
| MSM0657 | Msp_1299 | ApgM2 | 6.1E−150 | MTH_1591 | phosphonopyruvate | 6.0E−148 |
| | | | | | decarboxylase | |
| MSM0658 | NONE | | | NONE | | |
| MSM0659 | Msp_1298 | conserved hypothetical | 4.8E−63 | MTH_1592 | conserved protein | 1.1E−77 |
| | | membrane-spanning protein | | | | |
| MSM0660 | Msp_1568 | conserved hypothetical | 3.9E−52 | NONE | | |
| | | membrane-spanning protein | | | | |
| MSM0661 | Msp_1297 | 30S ribosomal protein S3Ae | 3.2E−66 | MTH_1593 | ribosomal protein S3a | 8.4E−71 |
| MSM0662 | Msp_0712 | hypothetical membrane-spanning | 8.9E−07 | NONE | | |
| | | protein | | | | |
| MSM0663 | Msp_1295 | predicted iron-molybdenum | 1.4E−08 | MTH_1594 | conserved protein | 1.2E−16 |
| | | cluster-binding protein | | | | |
| MSM0664 | Msp_0540 | predicted multimeric flavodoxin | 2.4E−22 | MTH_1595 | conserved protein | 5.0E−57 |
| MSM0665 | Msp_0642 | predicted purine nucleoside | 7.4E−74 | MTH_1596 | methylthioadenosine | 3.7E−77 |
| | | phosphorylase | | | phosphorylase | |
| MSM0666 | Msp_0641 | conserved hypothetical | 6.7E−176 | MTH_1597 | conserved protein | 3.5E−184 |
| | | membrane-spanning protein | | | | |
| MSM0667 | Msp_0587 | hypothetical membrane-spanning | 1.8E−05 | MTH_520 | unknown | 3.7E−13 |
| | | protein | | | | |
| MSM0668 | Msp_0637 | conserved hypothetical protein | 4.9E−22 | MTH_1598 | conserved protein | 5.8E−40 |
| MSM0669 | NONE | | | NONE | | |
| MSM0670 | NONE | | | NONE | | |
| MSM0671 | Msp_0635 | cell division control protein 6-like 2 | 2.7E−108 | MTH_1599 | Cdc6 related protein | 5.4E−131 |
| MSM0672 | Msp_0661 | conserved hypothetical protein | 1.4E−56 | MTH_1600 | conserved protein | 7.0E−67 |
| MSM0673 | Msp_1557 | conserved hypothetical | 5.1E−27 | NONE | | |
| | | membrane-spanning protein | | | | |
| MSM0674 | NONE | | | NONE | | |
| MSM0675 | NONE | | | NONE | | |
| MSM0676 | Msp_1557 | conserved hypothetical | 9.7E−33 | NONE | | |
| | | membrane-spanning protein | | | | |
| MSM0677 | Msp_0662 | putative aspartate | 1.3E−131 | MTH_1601 | aspartate | 7.3E−136 |
| | | aminotransferase | | | aminotransferase | |
| MSM0678 | Msp_0505 | conserved hypothetical | 8.1E−29 | MTH_519 | unknown | 1.1E−20 |
| | | membrane-spanning protein | | | | |
| MSM0679 | Msp_0587 | hypothetical membrane-spanning | 8.1E−12 | MTH_520 | unknown | 8.1E−34 |
| | | protein | | | | |
| MSM0680 | Msp_0757 | predicted ATPase | 2.4E−109 | NONE | | |
| MSM0681 | NONE | | | NONE | | |
| MSM0682 | NONE | | | NONE | | |
| MSM0683 | Msp_0380 | hypothetical protein | 3.1E−13 | MTH_626 | unknown | 9.7E−22 |
| MSM0684 | Msp_0381 | hypothetical membrane-spanning | 1.2E−09 | MTH_625 | unknown | 1.5E−04 |
| | | protein | | | | |
| MSM0685 | NONE | | | NONE | | |
| MSM0686 | Msp_0605 | predicted thiamine | 2.1E−94 | NONE | acetolactate synthase, | 8.5E−94 |
| | | pyrophosphate-requiring enzyme | | | large subunit homolog | |
| MSM0687 | Msp_0604 | predicted deoxycytidine | 1.6E−57 | MTH_1605 | deoxycytidine- | 8.2E−57 |
| | | triphosphate deaminase | | | triphosphate deaminase | |
| | | | | | related protein | |
| MSM0688 | Msp_1409 | predicted tautomerase | 3.2E−11 | MTH_1606 | unknown | 1.7E−08 |
| MSM0689 | NONE | | | NONE | | |
| MSM0690 | Msp_0767 | predicted helicase | 2.1E−243 | NONE | ATP-dependent RNA | 9.5E−09 |
| | | | | | helicase, eIF-4A family | |
| MSM0691 | Msp_0006 | predicted NUDIX-related protein | 1.4E−40 | MTH_1336 | mutator MutT protein | 4.1E−14 |
| | | | | | homolog | |
| MSM0692 | NONE | | | NONE | | |
| MSM0693 | Msp_0113 | conserved hypothetical protein | 1.4E−13 | MTH_540 | intracellular protein | 7.2E−10 |
| | | | | | transport protein | |
| MSM0694 | NONE | | | NONE | | |
| MSM0695 | Msp_0767 | predicted helicase | 1.0E−13 | NONE | ATP-dependent RNA | 3.7E−10 |
| | | | | | helicase, eIF-4A family | |
| MSM0696 | Msp_1095 | DNA double-strand break repair | 4.0E−04 | NONE | | |
| | | protein Rad50 | | | | |
| MSM0697 | NONE | | | NONE | | |
| MSM0698 | NONE | | | NONE | | |
| MSM0699 | Msp_0738 | predicted Na+-dependent | 4.1E−137 | MTH_1909 | unknown | 5.8E−04 |
| | | transporter | | | | |
| MSM0700 | Msp_0921 | putative poly-gamma-glutamate | 1.0E−108 | NONE | | |
| | | biosynthesis protein | | | | |
| MSM0701 | Msp_0601 | partially conserved hypothetical | 2.4E−116 | MTH_1608 | signal recognition particle | 3.6E−111 |
| | | protein, predicted GTPase | | | protein (docking protein) | |
| MSM0702 | Msp_0600 | conserved hypothetical protein | 1.5E−20 | MTH_1609 | conserved protein | 1.1E−36 |
| MSM0703 | Msp_0599 | RplX | 4.1E−18 | MTH_1610 | ribosomal protein L18a | 1.0E−17 |
| MSM0704 | Msp_0598 | translation initiation factor 6 (aIF- | 3.7E−56 | MTH_1611 | conserved protein | 3.8E−59 |
| | | 6) | | | | |
| MSM0705 | Msp_0597 | 50S ribosomal protein L31e | 1.4E−22 | MTH_1612 | ribosomal protein L31 | 4.7E−29 |
| MSM0706 | NONE | | | MTH_1613 | ribosomal protein L39 | 1.2E−16 |
| MSM0707 | Msp_0596 | predicted subunit of tRNA | 2.8E−58 | MTH_1614 | conserved protein | 3.8E−59 |
| | | methyltransferase | | | | |
| MSM0708 | Msp_0595 | partially conserved hypothetical | 1.4E−31 | MTH_1615 | conserved protein | 3.1E−32 |
| | | protein | | | | |
| MSM0709 | Msp_0594 | 30S ribosomal protein S19e | 1.5E−52 | MTH_1616 | ribosomal protein S19 | 5.9E−54 |
| MSM0710 | Msp_0593 | hypothetical protein | 1.3E−28 | MTH_1617 | conserved protein | 1.3E−19 |
| MSM0711 | Msp_0592 | putative ribonuclease P, subunit 4 | 8.7E−32 | MTH_1618 | conserved protein | 3.0E−34 |
| MSM0712 | NONE | | | NONE | | |
| MSM0713 | Msp_0589 | predicted nucleotide kinase | 3.1E−36 | MTH_1619 | conserved protein | 2.4E−34 |
| | | | | | (adenylate kinase | |
| | | | | | related) | |
| MSM0714 | Msp_0660 | predicted GTPase | 2.1E−46 | NONE | GTP-binding protein, | 3.9E−50 |
| | | | | | GTP1/OBG family | |
| MSM0715 | Msp_0660 | predicted GTPase | 2.4E−77 | NONE | GTP-binding protein, | 1.2E−87 |
| | | | | | GTP1/OBG family | |
| MSM0716 | Msp_0368 | conserved hypothetical | 1.1E−141 | MTH_1623 | oligosaccharyl | 7.3E−88 |
| | | membrane-spanning protein | | | transferase STT3 subunit | |
| | | | | | related protein | |
| MSM0717 | Msp_0366 | TopA | 8.0E−228 | MTH_1624 | DNA topoisomerase I | 3.1E−247 |
| MSM0718 | NONE | | | MTH_1625 | unknown | 4.6E−15 |
| MSM0719 | Msp_1096 | putative phosphoserine | 2.7E−124 | MTH_1626 | phosphoserine | 1.3E−83 |
| | | phosphatase | | | phosphatase | |
| MSM0720 | Msp_1097 | TATA-box binding protein | 5.0E−68 | MTH_1627 | TATA-binding | 1.2E−73 |
| | | | | | transcription initiation | |
| | | | | | factor | |
| MSM0721 | Msp_1098 | predicted adenylate cyclase | 2.6E−39 | MTH_1629 | conserved protein | 1.3E−42 |
| MSM0722 | Msp_1099 | LeuA2 | 1.9E−91 | MTH_1630 | 2-isopropylmalate | 1.5E−151 |
| | | | | | synthase | |
| MSM0723 | Msp_1100 | LeuC2 | 2.7E−140 | NONE | 3-isopropylmalate | 5.8E−150 |
| | | | | | dehydratase, LeuC | |
| | | | | | subunit | |
| MSM0724 | Msp_0326 | hypothetical protein | 9.1E−04 | MTH_1632 | conserved protein | 1.0E−40 |
| MSM0725 | Msp_1086 | flap structure-specific | 9.2E−92 | MTH_1633 | DNA repair protein Rad2 | 7.8E−100 |
| | | endonuclease | | | | |
| MSM0726 | NONE | | | MTH_1635 | conserved protein | 7.1E−42 |
| MSM0727 | Msp_1085 | AhcY | 1.3E−163 | MTH_1636 | S-adenosylhomocysteine | 3.7E−164 |
| | | | | | hydrolase | |
| MSM0728 | Msp_0524 | predicted oxidoreductase | 4.4E−92 | MTH_907 | conserved protein | 2.5E−62 |
| MSM0729 | Msp_0231 | predicted E1-like enzyme | 2.1E−46 | MTH_1571 | molybdopterin | 1.7E−65 |
| | | | | | biosynthesis protein | |
| | | | | | MoeB homolog | |
| MSM0730 | Msp_0017 | conserved hypothetical protein | 1.7E−28 | NONE | | |
| MSM0731 | Msp_0113 | conserved hypothetical protein | 1.6E−13 | MTH_511 | DNA helicase II | 4.6E−07 |
| MSM0732 | Msp_0873 | TruB | 3.2E−105 | MTH_32 | centromere/microtubule- | 3.2E−110 |
| | | | | | binding protein | |
| MSM0733 | Msp_0880 | 50S ribosomal protein L14e | 2.3E−24 | MTH_31 | ribosomal protein L14 | 4.1E−23 |
| MSM0734 | Msp_0881 | putative cytidylate kinase | 1.8E−56 | MTH_30 | cytidylate kinase | 3.8E−52 |
| MSM0735 | Msp_0882 | 50S ribosomal protein L34e | 2.4E−29 | MTH_29 | ribosomal protein L34 | 3.3E−37 |
| | | | | | (E. coli) | |
| MSM0736 | Msp_0883 | hypothetical membrane-spanning | 1.2E−34 | MTH_28 | conserved protein | 1.1E−50 |
| | | protein | | | | |
| MSM0737 | Msp_0884 | AdkA | 1.1E−61 | MTH_27 | adenylate kinase | 1.1E−63 |
| MSM0738 | Msp_0885 | SecY | 6.6E−153 | MTH_26 | preprotein translocase | 1.0E−145 |
| | | | | | SecY | |
| MSM0739 | Msp_0886 | 50S ribosomal protein L15P | 1.9E−43 | MTH_25 | ribosomal protein L27a | 4.1E−46 |
| | | | | | (E. coli) | |
| MSM0740 | Msp_0887 | 50S ribosomal protein L30P | 9.7E−49 | MTH_24 | ribosomal protein L7 | 1.2E−53 |
| | | | | | (E. coli) | |
| MSM0741 | Msp_0888 | 30S ribosomal protein S5P | 3.5E−92 | MTH_23 | ribosomal protein S2 | 3.7E−93 |
| | | | | | (E. coli) | |
| MSM0742 | Msp_0889 | 50S ribosomal protein L18P | 6.7E−57 | MTH_22 | ribosomal protein L5 | 8.9E−67 |
| MSM0743 | Msp_0890 | 50S ribosomal protein L19e | 4.6E−58 | MTH_21 | ribosomal protein L19 | 1.5E−64 |
| MSM0744 | Msp_0891 | 50S ribosomal protein L32e | 6.6E−34 | MTH_20 | ribosomal protein L32 | 3.1E−41 |
| MSM0745 | Msp_0892 | 50S ribosomal protein L6P | 5.7E−60 | MTH_19 | ribosomal protein L9 | 4.3E−67 |
| | | | | | (E. coli) | |
| MSM0746 | Msp_0893 | 30S ribosomal protein S8P | 9.5E−58 | MTH_18 | ribosomal protein S15a | 1.2E−55 |
| | | | | | (E. coli) | |
| MSM0747 | Msp_0894 | 30S ribosomal protein S14P | 2.1E−21 | MTH_17 | ribosomal protein S29 | 7.6E−22 |
| | | | | | (E. coli) | |
| MSM0748 | Msp_0895 | 50S ribosomal protein L5P | 2.4E−61 | MTH_16 | ribosomal protein L11 | 2.9E−61 |
| | | | | | (E. coli) | |
| MSM0749 | Msp_0896 | 30S ribosomal protein S4e | 3.0E−70 | MTH_15 | ribosomal protein S4 | 1.8E−77 |
| MSM0750 | Msp_0897 | 50S ribosomal protein L24P | 2.4E−29 | MTH_14 | ribosomal protein L26 | 1.3E−35 |
| | | | | | (E. coli) | |
| MSM0751 | Msp_0898 | 50S ribosomal protein L14P | 1.4E−56 | MTH_13 | ribosomal protein L23 | 1.0E−56 |
| | | | | | (E. coli) | |
| MSM0752 | Msp_0899 | 30S ribosomal protein S17P | 1.4E−42 | MTH_12 | ribosomal protein S11 | 1.4E−45 |
| | | | | | (E. coli) | |
| MSM0753 | Msp_0900 | putative ribonuclease P, | 4.8E−24 | MTH_11 | conserved protein | 8.7E−21 |
| | | component 1 | | | | |
| MSM0754 | Msp_0901 | protein translation factor SUI1-like | 2.4E−45 | MTH_10 | ribosomal protein SUI1 | 3.6E−47 |
| | | protein | | | | |
| MSM0755 | Msp_0902 | 50S ribosomal protein L29P | 3.3E−16 | MTH_9 | ribosomal protein L35 | 7.9E−20 |
| | | | | | (E. coli) | |
| MSM0756 | Msp_0903 | 30S ribosomal protein S3P | 6.8E−96 | MTH_8 | ribosomal protein S3 | 1.2E−96 |
| | | | | | (E. coli) | |
| MSM0757 | Msp_0904 | 50S ribosomal protein L22P | 1.3E−46 | MTH_7 | ribosomal protein L17 | 3.5E−56 |
| | | | | | (E. coli) | |
| MSM0758 | Msp_0905 | 30S ribosomal protein S19P | 1.4E−58 | MTH_6 | ribosomal protein S15 | 1.3E−58 |
| | | | | | (E. coli) | |
| MSM0759 | Msp_0906 | 50S ribosomal protein L2P | 3.1E−107 | MTH_5 | ribosomal protein L8 | 1.9E−105 |
| | | | | | (E. coli) | |
| MSM0760 | Msp_0907 | 50S ribosomal protein L23P | 2.8E−26 | MTH_4 | ribosomal protein L23a | 5.4E−28 |
| | | | | | (E. coli) | |
| MSM0761 | Msp_0908 | 50S ribosomal protein L1e | 4.5E−99 | MTH_3 | ribosomal protein L4 | 2.6E−99 |
| | | | | | (E. coli) | |
| MSM0762 | Msp_0909 | 50S ribosomal protein L3P | 1.5E−121 | MTH_2 | ribosomal protein L3 | 1.1E−132 |
| | | | | | (E. coli) | |
| MSM0763 | Msp_0910 | conserved hypothetical protein | 1.1E−79 | MTH_1 | conserved protein | 1.2E−73 |
| MSM0764 | Msp_1319 | predicted DNA modification | 1.7E−04 | MTH_1918 | possible protein | 3.7E−45 |
| | | methylase | | | methyltransferase | |
| MSM0765 | Msp_0914 | PycA | 1.7E−186 | MTH_1917 | biotin carboxylase | 5.5E−202 |
| MSM0766 | Msp_0915 | partially conserved hypothetical | 4.0E−36 | MTH_1916 | biotin acetyl-CoA | 5.3E−62 |
| | | protein | | | carboxylase ligase/biotin | |
| | | | | | operon repressor | |
| MSM0767 | Msp_0916 | predicted selenocysteine | 2.8E−99 | MTH_1914 | conserved protein | 2.3E−100 |
| | | synthase | | | | |
| MSM0768 | Msp_0917 | hypothetical protein | 7.5E−04 | MTH_1912 | unknown | 1.1E−11 |
| MSM0769 | Msp_0791 | fumarate hydratase | 3.1E−59 | NONE | fumarate hydratase, class | 1.5E−50 |
| | | | | | I related protein | |
| MSM0770 | Msp_1112 | CbiO2 | 1.2E−43 | NONE | methyl coenzyme M | 8.3E−64 |
| | | | | | reductase system, | |
| | | | | | component A2 homolog | |
| MSM0771 | Msp_0657 | CbiQ2 | 1.4E−05 | MTH_453 | conserved protein | 2.6E−12 |
| MSM0772 | NONE | | | MTH_452 | unknown | 9.2E−07 |
| MSM0773 | Msp_0958 | predicted ABC-type polar amino | 1.4E−26 | MTH_1704 | cobalt transport ATP- | 5.9E−25 |
| | | acid transport system, ATP- | | | binding protein O | |
| | | binding protein | | | | |
| MSM0774 | Msp_0340 | PstB | 1.6E−26 | MTH_1731 | phosphate transport | 5.2E−26 |
| | | | | | system ATP-binding | |
| MSM0775 | Msp_0149 | predicted transcriptional regulator | 2.0E−34 | NONE | | |
| MSM0776 | Msp_0790 | conserved hypothetical | 2.2E−138 | MTH_1909 | unknown | 2.8E−159 |
| | | membrane-spanning protein | | | | |
| MSM0777 | Msp_0491 | hypothetical membrane-spanning | 3.6E−10 | MTH_1908 | unknown | 3.2E−16 |
| | | protein | | | | |
| MSM0778 | Msp_0517 | predicted RNA-binding protein | 3.6E−184 | MTH_1907 | conserved protein | 2.0E−188 |
| MSM0779 | Msp_0516 | predicted Zn-dependent | 2.3E−70 | MTH_1902 | conserved protein | 3.5E−72 |
| | | hydrolase of the beta-lactamase | | | | |
| | | superfamily | | | | |
| MSM0780 | NONE | | | MTH_1901 | unknown | 2.9E−16 |
| MSM0781 | Msp_1151 | hypothetical membrane-spanning | 1.2E−09 | MTH_1533 | unknown | 1.3E−10 |
| | | protein | | | | |
| MSM0782 | Msp_1151 | hypothetical membrane-spanning | 2.4E−04 | MTH_979 | unknown | 1.2E−05 |
| | | protein | | | | |
| MSM0783 | Msp_1447 | EhbK | 3.3E−20 | NONE | tungsten | 3.5E−88 |
| | | | | | formylmethanofuran | |
| | | | | | dehydrogenase, subunit | |
| | | | | | F homolog | |
| MSM0784 | Msp_0236 | ferredoxin | 5.5E−14 | MTH_927 | ferredoxin | 5.1E−16 |
| MSM0785 | Msp_0514 | putative phosphopantetheine | 1.0E−37 | MTH_1896 | conserved protein | 1.3E−42 |
| | | adenylyltransferase | | | | |
| MSM0786 | Msp_1129 | partially conserved hypothetical | 1.1E−49 | MTH_412 | conserved protein | 1.3E−69 |
| | | membrane-spanning protein | | | | |
| MSM0787 | Msp_0511 | predicted Fe—S oxidoreductase | 7.6E−120 | MTH_1895 | conserved protein | 8.7E−124 |
| MSM0788 | Msp_0510 | putative aspartate | 5.5E−117 | MTH_1894 | aspartate | 3.3E−108 |
| | | aminotransferase | | | aminotransferase | |
| | | | | | homolog | |
| MSM0789 | Msp_0519 | predicted Co/Zn/Cd cation | 7.6E−33 | MTH_1893 | cation efflux system | 1.8E−77 |
| | | transporter | | | protein (zinc/cadmium) | |
| MSM0790 | Msp_1428 | conserved hypothetical protein | 1.3E−15 | MTH_1884 | conserved protein | 3.0E−36 |
| MSM0791 | Msp_0443 | 2-phosphoglycerate kinase | 3.6E−81 | MTH_1883 | 2-phosphoglycerate | 3.7E−84 |
| | | | | | kinase | |
| MSM0792 | Msp_1010 | predicted phosphoesterase | 1.8E−47 | MTH_1882 | conserved protein | 2.3E−52 |
| MSM0793 | Msp_1011 | conserved hypothetical protein | 1.9E−29 | MTH_1881 | conserved protein | 4.4E−42 |
| MSM0794 | Msp_1012 | conserved hypothetical protein | 1.9E−20 | MTH_1880 | conserved protein | 2.1E−28 |
| MSM0795 | Msp_1013 | HdrB1 | 1.9E−116 | NONE | heterodisulfide reductase, | 4.3E−115 |
| | | | | | subunit B | |
| MSM0796 | Msp_1014 | HdrC1 | 1.6E−69 | NONE | heterodisulfide reductase, | 4.7E−77 |
| | | | | | subunit C | |
| MSM0797 | Msp_1015 | conserved hypothetical protein | 2.5E−50 | MTH_1877 | conserved protein | 1.6E−53 |
| MSM0798 | NONE | | | NONE | | |
| MSM0799 | Msp_0113 | conserved hypothetical protein | 1.6E−12 | MTH_1626 | phosphoserine | 2.2E−06 |
| | | | | | phosphatase | |
| MSM0800 | NONE | | | NONE | | |
| MSM0801 | Msp_1017 | DphB | 1.7E−74 | MTH_1874 | diphthine synthase | 2.9E−77 |
| MSM0802 | Msp_1022 | predicted methyltransferase | 3.6E−81 | MTH_1873 | met-10+ protein | 1.3E−74 |
| MSM0803 | NONE | | | MTH_633 | conserved protein | 4.3E−04 |
| MSM0804 | Msp_1023 | putative translation initiation factor | 5.0E−100 | NONE | translation initiation factor | 2.2E−125 |
| | | aIF-2B, subunit 1 | | | eIF-2B, alpha subunit | |
| MSM0805 | Msp_0958 | predicted ABC-type polar amino | 5.0E−100 | MTH_696 | ABC transporter | 2.7E−35 |
| | | acid transport system, ATP- | | | (glutamine transport ATP- | |
| | | binding protein | | | binding protein) | |
| MSM0806 | Msp_0959 | predicted ABC-type polar amino | 2.1E−92 | NONE | | |
| | | acid transport system, permease | | | | |
| | | protein | | | | |
| MSM0807 | Msp_0960 | predicted ABC-type polar amino | 3.5E−108 | NONE | | |
| | | acid transport system, periplasmic | | | | |
| | | substrate-binding protein | | | | |
| MSM0808 | Msp_1024 | conserved hypothetical protein | 2.9E−104 | MTH_1871 | nitrogenase iron- | 1.6E−115 |
| | | | | | molybdenum cofactor | |
| | | | | | biosynthesis protein NifB | |
| MSM0809 | Msp_1025 | conserved hypothetical protein | 2.3E−40 | MTH_1870 | conserved protein | 3.1E−41 |
| MSM0810 | Msp_1026 | predicted activator of 2- | 5.5E−165 | MTH_1869 | activator of (R)-2- | 1.7E−175 |
| | | hydroxyglutaryl-CoA dehydratase | | | hydroxyglutaryl-CoA | |
| MSM0811 | Msp_1027 | conserved hypothetical protein | 1.7E−53 | MTH_1868 | conserved protein | 1.2E−57 |
| MSM0812 | Msp_1029 | conserved hypothetical protein | 1.3E−39 | MTH_1866 | conserved protein | 1.0E−40 |
| MSM0813 | Msp_1030 | predicted peptidyl-prolyl cis-trans | 2.6E−135 | MTH_1865 | conserved protein | 2.3E−146 |
| | | isomerase | | | | |
| MSM0814 | Msp_1032 | predicted selenophosphate | 3.3E−87 | MTH_1864 | phosphoribosylformylglycinamidine | 6.2E−91 |
| | | synthetase-related protein | | | synthase II | |
| | | | | | related protein | |
| MSM0815 | Msp_1033 | conserved hypothetical protein | 4.5E−99 | MTH_1863 | conserved protein | 4.4E−97 |
| MSM0816 | Msp_1034 | predicted nucleic acid-binding | 3.7E−33 | MTH_1862 | conserved protein | 3.5E−40 |
| | | protein | | | | |
| MSM0817 | Msp_0799 | predicted transcriptional regulator | 6.6E−34 | MTH_1843 | unknown | 1.0E−33 |
| MSM0818 | Msp_0798 | predicted transcriptional regulator | 5.0E−36 | MTH_1843 | unknown | 2.1E−26 |
| MSM0819 | NONE | | | MTH_1438 | unknown | 4.6E−15 |
| MSM0820 | NONE | | | MTH_1861 | molybdenum cofactor | 2.5E−46 |
| | | | | | biosynthesis MoaB | |
| MSM0821 | Msp_1036 | PyrE | 3.1E−59 | MTH_1860 | uridine 5′- | 5.2E−55 |
| | | | | | monophosphate synthase | |
| MSM0822 | Msp_1035 | hypothetical protein | 3.1E−13 | MTH_1859 | unknown | 1.4E−15 |
| MSM0823 | NONE | | | NONE | | |
| MSM0824 | NONE | | | NONE | N-terminal | 3.1E−06 |
| | | | | | acetyltransferase | |
| | | | | | complex, subunit ARD1 | |
| MSM0825 | Msp_0437 | conserved hypothetical protein | 4.7E−56 | NONE | | |
| MSM0826 | Msp_0114 | ThsB | 8.2E−226 | MTH_794 | chaperonin | 2.4E−231 |
| MSM0827 | Msp_0747 | member of asn/thr-rich large | 5.9E−04 | MTH_796 | conserved protein | 4.5E−33 |
| | | protein family | | | | |
| MSM0828 | Msp_0220 | predicted glycosyltransferase | 2.0E−14 | MTH_540 | intracellular protein | 8.1E−06 |
| | | | | | transport protein | |
| MSM0829 | Msp_0110 | aspartate-semialdehyde | 6.6E−121 | MTH_799 | aspartate-semialdehyde | 2.3E−132 |
| | | dehydrogenase | | | dehydrogenase | |
| MSM0830 | Msp_0109 | DapB | 1.0E−85 | MTH_800 | dihydrodipicolinate | 3.2E−87 |
| | | | | | reductase | |
| MSM0831 | Msp_0108 | DapA | 4.9E−86 | MTH_801 | dihydrodipicolinate | 2.0E−85 |
| | | | | | synthase | |
| MSM0832 | Msp_0107 | putative aspartokinase | 2.2E−129 | MTH_802 | aspartokinase II alpha | 6.7E−149 |
| | | | | | subunit | |
| MSM0833 | Msp_0106 | 30S ribosomal protein S17e | 1.3E−19 | MTH_803 | ribosomal protein S17 | 1.5E−23 |
| MSM0834 | Msp_0105 | putative chorismate mutase | 3.8E−15 | NONE | chorismate mutase, | 9.3E−17 |
| | | | | | subunit A | |
| MSM0835 | Msp_0104 | AroK | 4.7E−56 | MTH_805 | conserved protein | 2.6E−76 |
| | | | | | (homoserine kinase | |
| | | | | | related) | |
| MSM0836 | Msp_0101 | predicted glycosyltransferase | 2.6E−64 | MTH_450 | LPS biosynthesis RfbU | 9.6E−31 |
| | | | | | related protein | |
| MSM0837 | Msp_0102 | CbiD | 6.5E−91 | MTH_808 | cobalamin biosynthesis | 4.0E−87 |
| | | | | | protein D | |
| MSM0838 | Msp_0103 | putative thioredoxin | 2.5E−18 | MTH_807 | thioredoxin | 7.1E−19 |
| MSM0839 | Msp_0100 | predicted helicase | 2.1E−227 | MTH_810 | DNA helicase related | 9.1E−248 |
| | | | | | protein | |
| MSM0840 | Msp_0097 | conserved hypothetical protein | 3.0E−15 | MTH_814 | conserved protein | 1.6E−14 |
| MSM0841 | Msp_0371 | hypothetical protein | 6.6E−11 | MTH_815 | unknown | 2.2E−15 |
| MSM0842 | Msp_0372 | predicted histone | 1.5E−187 | MTH_817 | conserved protein | 6.2E−189 |
| | | acetyltransferase | | | | |
| MSM0843 | NONE | | | MTH_818 | deoxyribose-phosphate | 2.1E−26 |
| | | | | | aldolase | |
| MSM0844 | Msp_0122 | archaeal histone | 3.5E−21 | MTH_821 | histone HMtA1 | 2.5E−23 |
| MSM0845 | Msp_0376 | predicted 2-methylthioadenine | 8.9E−126 | MTH_826 | conserved protein | 3.8E−130 |
| | | synthetase | | | | |
| MSM0846 | Msp_0375 | conserved hypothetical protein | 1.6E−39 | MTH_828 | conserved protein | 1.6E−46 |
| MSM0847 | Msp_0374 | LeuD2 | 4.1E−57 | NONE | 3-isopropylmalate | 7.4E−56 |
| | | | | | dehydratase, LeuD | |
| | | | | | subunit | |
| MSM0848 | Msp_0373 | predicted archaeal sugar kinase | 1.5E−73 | MTH_830 | conserved protein | 3.0E−82 |
| MSM0849 | Msp_0384 | predicted Fe—S oxidoreductase | 6.6E−169 | MTH_831 | molybdenum cofactor | 2.7E−177 |
| | | | | | biosynthesis MoaA | |
| | | | | | homolog | |
| MSM0850 | Msp_0385 | conserved hypothetical | 2.4E−45 | MTH_832 | conserved protein | 1.4E−43 |
| | | membrane-spanning protein | | | | |
| MSM0851 | Msp_0386 | predicted transcriptional regulator | 1.1E−70 | MTH_834 | conserved protein | 3.0E−98 |
| MSM0852 | Msp_0387 | predicted ATP-utilizing enzyme | 2.3E−40 | MTH_835 | conserved protein | 1.0E−53 |
| MSM0853 | Msp_0217 | predicted UDP-N- | 1.4E−120 | MTH_837 | UDP-N- | 1.3E−136 |
| | | acetylglucosamine 2-epimerase | | | acetylglucosamine 2- | |
| | | | | | epimerase | |
| MSM0854 | NONE | | | NONE | | |
| MSM0855 | Msp_0388 | TruA | 5.2E−50 | MTH_840 | pseudouridylate synthase I | 1.6E−51 |
| MSM0856 | NONE | | | MTH_695 | conserved protein | 1.7E−08 |
| MSM0857 | Msp_1000 | predicted ABC-type | 1.5E−29 | MTH_696 | ABC transporter | 3.3E−44 |
| | | nitrate/sulfonate/bicarbonate | | | (glutamine transport ATP- | |
| | | transport system, ATB-binding | | | binding protein) | |
| | | protein | | | | |
| MSM0858 | Msp_0389 | HisA | 6.3E−77 | MTH_843 | phosphoribosylformimino- | 7.4E−79 |
| | | | | | 5-aminoimidazole | |
| | | | | | carboxamide ribotide | |
| | | | | | isomerase | |
| MSM0859 | Msp_0390 | putative cytidylyltransferase | 5.1E−43 | MTH_844 | autotrophic growth | 1.5E−48 |
| | | | | | protein | |
| MSM0860 | Msp_0552 | ArgC | 4.9E−109 | MTH_846 | N-acetyl-gamma-glutamyl- | 2.0E−108 |
| | | | | | phosphate reductase | |
| MSM0861 | Msp_0554 | hypothetical protein | 4.8E−31 | MTH_847 | unknown | 3.3E−44 |
| MSM0862 | Msp_0521 | PyrI | 2.1E−44 | MTH_850 | aspartate | 7.5E−47 |
| | | | | | carbamoyltransferase | |
| | | | | | regulatory subunit | |
| MSM0863 | Msp_1419 | hypothetical protein | 3.1E−20 | NONE | | |
| MSM0864 | NONE | | | MTH_1285 | conserved protein | 2.7E−10 |
| MSM0865 | Msp_0159 | conserved hypothetical protein | 1.1E−79 | MTH_853 | conserved protein | 2.4E−96 |
| MSM0866 | Msp_0402 | predicted zinc metalloprotease | 4.7E−143 | MTH_856 | zinc metalloproteinase | 8.2E−144 |
| MSM0867 | Msp_0403 | conserved hypothetical protein | 1.1E−47 | MTH_857 | conserved protein | 4.0E−48 |
| MSM0868 | NONE | | | NONE | | |
| MSM0869 | Msp_0404 | predicted GTPase | 3.0E−93 | NONE | GTP-binding protein, | 8.2E−112 |
| | | | | | GTP1/OBG family | |
| MSM0870 | Msp_0405 | putative small heat shock protein | 1.2E−16 | NONE | heat shock protein, class I | 3.8E−20 |
| MSM0871 | Msp_0017 | conserved hypothetical protein | 1.7E−28 | NONE | | |
| MSM0872 | Msp_1054 | predicted phosphosugar | 1.2E−103 | MTH_860 | glucosamine--fructose-6- | 5.6E−113 |
| | | isomerase | | | phosphate | |
| | | | | | aminotransferase | |
| MSM0873 | Msp_1309 | conserved hypothetical protein | 7.6E−17 | MTH_863 | conserved protein | 5.4E−28 |
| MSM0874 | Msp_1308 | adenine deaminase | 1.5E−139 | MTH_866 | adenine deaminase | 1.3E−132 |
| MSM0875 | Msp_1347 | conserved hypothetical protein | 6.0E−136 | MTH_867 | conserved protein | 6.4E−144 |
| MSM0876 | Msp_0415 | predicted | 1.3E−71 | MTH_868 | agmatine ureohydrolase | 1.2E−73 |
| | | arginase/agmatinase/formimionoglutamate | | | | |
| | | hydrolase | | | | |
| MSM0877 | Msp_1352 | translation initiation factor 5A (aIF- | 4.4E−53 | NONE | translation initiation factor, | 1.7E−49 |
| | | 5A) | | | eIF-5A | |
| MSM0878 | Msp_1327 | PdaD | 2.1E−37 | MTH_870 | conserved protein | 3.4E−42 |
| MSM0879 | Msp_1330 | PpnK | 7.2E−60 | MTH_872 | conserved protein | 9.0E−77 |
| MSM0880 | Msp_1331 | predicted UDP-N-acetylmuramyl | 1.1E−47 | MTH_873 | UDP-N-acetylmuramyl | 5.4E−81 |
| | | pentapeptide synthase | | | tripeptide synthetase | |
| | | | | | related protein | |
| MSM0881 | Msp_1332 | HemC | 7.3E−83 | MTH_874 | porphobilinogen | 2.0E−85 |
| | | | | | deaminase | |
| MSM0882 | Msp_1333 | predicted dehydrogenase | 2.7E−101 | NONE | 3-chlorobenzoate-3,4- | 3.0E−130 |
| | | | | | dioxygenase | |
| | | | | | dyhydrogenase related | |
| | | | | | protein | |
| MSM0883 | Msp_1334 | predicted orotate | 5.6E−53 | MTH_876 | orotate | 9.7E−70 |
| | | phosphoribosyltransferase | | | phosphoribosyltransferase | |
| MSM0884 | Msp_0747 | member of asn/thr-rich large | 1.5E−18 | MTH_716 | cell surface glycoprotein | 4.1E−07 |
| | | protein family | | | (s-layer protein) | |
| MSM0885 | Msp_1465 | member of asn/thr-rich large | 2.4E−39 | MTH_716 | cell surface glycoprotein | 1.7E−08 |
| | | protein family | | | (s-layer protein) | |
| MSM0886 | NONE | | | NONE | | |
| MSM0887 | Msp_1410 | predicted universal stress protein | 2.5E−18 | MTH_898 | conserved protein | 1.5E−18 |
| MSM0888 | Msp_1416 | GdhA | 2.6E−181 | NONE | | |
| MSM0889 | NONE | | | NONE | | |
| MSM0890 | NONE | | | NONE | | |
| MSM0891 | Msp_1363 | peptide chain release factor, | 3.4E−149 | NONE | peptide chain release | 8.7E−156 |
| | | subunit 1 (aRF-1) | | | factor eRF, subunit 1 | |
| MSM0892 | Msp_1056 | hypothetical membrane-spanning | 5.4E−06 | MTH_1905 | unknown | 3.2E−06 |
| | | protein | | | | |
| MSM0893 | Msp_1202 | predicted acetyltransferase | 2.4E−29 | NONE | N-terminal | 3.7E−38 |
| | | | | | acetyltransferase | |
| | | | | | complex, subunit ARD1 | |
| MSM0894 | Msp_1203 | conserved hypothetical protein | 5.7E−28 | MTH_1000 | conserved protein | 1.2E−25 |
| MSM0895 | Msp_1204 | predicted cation transport ATPase | 3.9E−235 | MTH_1001 | cation-transporting P- | 9.8E−251 |
| | | | | | ATPase PacL | |
| MSM0896 | Msp_1205 | CbiJ | 6.5E−43 | MTH_1002 | cobalamin biosynthesis | 8.5E−39 |
| | | | | | protein J | |
| MSM0897 | Msp_1365 | 30S ribosomal protein S10P | 1.6E−48 | MTH_1059 | ribosomal protein S20 | 1.3E−49 |
| | | | | | (E. coli) | |
| MSM0898 | Msp_1366 | translation elongation factor 1- | 1.9E−185 | NONE | translation elongation | 3.9E−192 |
| | | alpha (EF-Tu) | | | factor, EF-1 alpha | |
| MSM0899 | Msp_1367 | FusA | 1.7e−319 | NONE | translation elongation | 1.9e−318 |
| | | | | | factor, EF-2 | |
| MSM0900 | Msp_1368 | 30S ribosomal protein S7P | 3.3E−80 | MTH_1056 | ribosomal protein S5 | 9.2E−81 |
| | | | | | (E. coli) | |
| MSM0901 | Msp_1369 | 30S ribosomal protein S12P | 4.4E−69 | MTH_1055 | ribosomal protein S23 | 7.8E−68 |
| | | | | | (E. coli) | |
| MSM0902 | Msp_0321 | MrtA | 5.7E−250 | NONE | methyl coenzyme M | 2.0E−250 |
| | | | | | reductase II, alpha | |
| | | | | | subunit | |
| MSM0903 | Msp_0320 | MrtG | 1.6E−103 | NONE | methyl coenzyme M | 1.8E−116 |
| | | | | | reductase II, gamma | |
| | | | | | subunit | |
| MSM0904 | Msp_0319 | MrtD | 1.9E−45 | NONE | methyl coenzyme M | 2.2E−40 |
| | | | | | reductase II, D protein | |
| MSM0905 | Msp_0318 | MrtB | 9.8E−159 | NONE | methyl coenzyme M | 4.1E−181 |
| | | | | | reductase II, beta subunit | |
| MSM0906 | Msp_1370 | NusA | 1.7E−44 | MTH_1054 | transcription termination | 2.5E−55 |
| | | | | | factor NusA | |
| MSM0907 | Msp_1371 | 50S ribosomal protein L30e | 6.0E−33 | MTH_1053 | ribosomal protein L30 | 3.0E−36 |
| MSM0908 | Msp_1372 | RpoA2 | 2.1E−126 | NONE | DNA-dependent RNA | 4.7E−141 |
| | | | | | polymerase, subunit A″ | |
| MSM0909 | Msp_1373 | RpoA1 | 0.0E+00 | NONE | DNA-dependent RNA | 0.0E+00 |
| | | | | | polymerase, subunit A′ | |
| MSM0910 | Msp_1374 | RpoB1 | 6.1E−253 | NONE | DNA-dependent RNA | 4.6E−276 |
| | | | | | polymerase, subunit B′ | |
| MSM0911 | Msp_1375 | RpoB2 | 3.3E−103 | NONE | DNA-dependent RNA | 8.6E−220 |
| | | | | | polymerase, subunit B″ | |
| MSM0912 | Msp_1376 | RpoH | 7.6E−17 | NONE | DNA-dependent RNA | 4.6E−15 |
| | | | | | polymerase, subunit H | |
| MSM0913 | NONE | | | NONE | | |
| MSM0914 | NONE | | | MTH_72 | O-linked GlcNAc | 3.0E−04 |
| | | | | | transferase | |
| MSM0915 | NONE | | | NONE | | |
| MSM0916 | Msp_0682 | ThiM1 | 1.2E−73 | NONE | | |
| MSM0917 | Msp_0683 | hypothetical protein | 7.7E−56 | NONE | | |
| MSM0918 | Msp_1381 | phosphoglycerate kinase | 1.1E−120 | MTH_1042 | 3-phosphoglycerate | 4.3E−131 |
| | | | | | kinase | |
| MSM0919 | Msp_1382 | TpiA | 4.9E−77 | MTH_1041 | triosephosphate | 3.2E−71 |
| | | | | | isomerase | |
| MSM0920 | Msp_1103 | member of asn/thr-rich large | 4.2E−04 | NONE | | |
| | | protein family | | | | |
| MSM0921 | Msp_0548 | hypothetical membrane-spanning | 1.1E−05 | NONE | | |
| | | protein | | | | |
| MSM0922 | Msp_1383 | predicted Fe—S oxidoreductase | 1.7E−97 | MTH_1039 | conserved protein | 4.9E−98 |
| MSM0923 | Msp_0540 | predicted multimeric flavodoxin | 1.2E−16 | MTH_135 | conserved protein | 1.3E−17 |
| MSM0924 | Msp_1386 | SucC | 3.4E−101 | NONE | succinyl-CoA synthetase, | 3.7E−116 |
| | | | | | beta subunit | |
| MSM0925 | Msp_1387 | KorC | 9.5E−58 | NONE | 2-oxoglutarate | 8.8E−60 |
| | | | | | oxidoreductase, gamma | |
| | | | | | subunit | |
| MSM0926 | Msp_1388 | KorB | 1.3E−99 | NONE | 2-oxoglutarate | 2.2E−102 |
| | | | | | oxidoreductase, beta | |
| | | | | | subunit | |
| MSM0927 | Msp_1389 | KorA | 4.5E−138 | NONE | 2-oxoglutarate | 6.2E−130 |
| | | | | | oxidoreductase, alpha | |
| | | | | | subunit | |
| MSM0928 | Msp_1390 | KorD | 3.0E−15 | NONE | ferredoxin (putative 2- | 8.6E−14 |
| | | | | | oxoglutarate | |
| | | | | | oxidoreductase, delat | |
| | | | | | subunit) | |
| MSM0929 | Msp_0791 | fumarate hydratase | 3.7E−17 | NONE | fumarate hydratase, class I | 3.5E−40 |
| MSM0930 | Msp_0325 | predicted peptidyl-prolyl cis-trans | 3.5E−67 | MTH_1125 | fkbp-type peptidyl-prolyl | 1.8E−77 |
| | | isomerase 2 | | | cis-trans isomerase | |
| MSM0931 | Msp_0801 | conserved hypothetical protein | 7.0E−94 | MTH_448 | unknown | 4.8E−68 |
| MSM0932 | Msp_1167 | conserved hypothetical protein | 4.7E−49 | MTH_1113 | conserved protein | 1.6E−58 |
| MSM0933 | Msp_1168 | CobS | 1.2E−50 | MTH_1112 | cobalamin (5′-phosphate) | 1.9E−41 |
| | | | | | synthase | |
| MSM0934 | Msp_1169 | hypothetical protein | 1.1E−06 | MTH_1111 | conserved protein | 1.5E−41 |
| MSM0935 | Msp_1170 | conserved hypothetical protein | 4.5E−106 | MTH_1109 | conserved protein | 4.2E−92 |
| MSM0936 | Msp_1171 | predicted ATPase | 6.3E−77 | MTH_1108 | conserved protein | 1.0E−65 |
| MSM0937 | NONE | | | NONE | | |
| MSM0938 | NONE | | | NONE | | |
| MSM0939 | Msp_1173 | PycB | 1.4E−212 | NONE | oxaloacetate | 2.8E−221 |
| | | | | | decarboxylase, alpha | |
| | | | | | subunit | |
| MSM0940 | Msp_1166 | predicted myo-inositol-1- | 5.3E−151 | MTH_1105 | conserved protein | 9.4E−159 |
| | | phosphate synthase | | | | |
| MSM0941 | Msp_0634 | predicted prenyltransferase | 2.3E−70 | MTH_1098 | bacteriochlorophyll | 4.2E−69 |
| | | | | | synthase related protein | |
| MSM0942 | Msp_0616 | partially conserved hypothetical | 5.0E−52 | MTH_371 | unknown | 5.1E−35 |
| | | membrane-spanning protein | | | | |
| MSM0943 | NONE | | | MTH_466 | unknown | 5.6E−09 |
| MSM0944 | NONE | | | NONE | | |
| MSM0945 | Msp_1285 | hydrogenase | 9.3E−147 | MTH_1072 | hydrogenase | 2.2E−141 |
| | | expression/formation protein | | | expression/formation | |
| | | | | | protein HypD | |
| MSM0946 | Msp_0215 | predicted glycosyltransferase | 6.1E−04 | MTH_1071 | conserved protein | 3.9E−50 |
| MSM0947 | Msp_1284 | predicted modulator of DNA | 3.7E−95 | MTH_1070 | conserved protein | 1.5E−96 |
| | | gyrase | | | | |
| MSM0948 | Msp_0220 | predicted glycosyltransferase | 4.0E−04 | NONE | | |
| MSM0949 | Msp_1351 | predicted transcriptional activator | 6.7E−18 | MTH_628 | unknown | 1.6E−19 |
| MSM0950 | NONE | | | MTH_1003 | molybdenum cofactor | 6.8E−101 |
| | | | | | biosynthesis protein MoeA | |
| MSM0951 | Msp_1335 | translation initiation factor 1A (aIF- | 1.6E−41 | NONE | translation initiation factor, | 1.3E−44 |
| | | 1A) (eIF1A) | | | eIF-1A | |
| MSM0952 | Msp_1337 | predicted serine/threonine protein | 5.1E−59 | MTH_1005 | conserved protein | 1.1E−75 |
| | | kinase | | | | |
| MSM0953 | NONE | | | MTH_630 | unknown | 1.5E−04 |
| MSM0954 | Msp_1338 | predicted RNA-binding protein | 1.4E−56 | MTH_1006 | conserved protein | 2.0E−60 |
| MSM0955 | Msp_1339 | type II DNA topoisomerase VI, | 2.4E−203 | MTH_1007 | conserved protein | 1.5E−213 |
| | | subunit B | | | | |
| MSM0956 | Msp_1340 | type II DNA topoisomerase VI, | 4.3E−149 | MTH_1008 | conserved protein | 1.8E−155 |
| | | subunit A | | | | |
| MSM0957 | Msp_0119 | hypothetical membrane-spanning | 6.8E−20 | MTH_524 | unknown | 4.9E−35 |
| | | protein | | | | |
| MSM0958 | Msp_1110 | CobN | 5.3E−11 | MTH_515 | unknown | 1.1E−08 |
| MSM0959 | Msp_0994 | conserved hypothetical protein | 3.0E−31 | NONE | | |
| MSM0960 | Msp_0678 | predicted cation transport ATPase | 4.8E−134 | MTH_411 | cadmium efflux ATPase | 1.9E−80 |
| MSM0961 | Msp_0224 | predicted cation transport ATPase | 9.6E−07 | MTH_1535 | heavy-metal transporting | 1.4E−08 |
| | | | | | CPx-type ATPase | |
| MSM0962 | Msp_1346 | glyceraldehyde 3-phosphate | 4.7E−127 | MTH_1009 | glyceraldehyde 3- | 5.9E−134 |
| | | dehydrogenase | | | phosphate | |
| | | | | | dehydrogenase | |
| MSM0963 | Msp_0992 | putative endonuclease IV | 9.5E−06 | MTH_1010 | endonuclease IV | 6.6E−71 |
| MSM0964 | Msp_1349 | predicted phosphohydrolase | 8.0E−19 | MTH_1179 | conserved protein | 1.1E−38 |
| MSM0965 | Msp_0718 | preducted 3-hydroxyacyl-CoA | 2.6E−126 | NONE | | |
| | | dehydrogenase | | | | |
| MSM0966 | Msp_1415 | putative 26S protease, regulatory | 6.5E−107 | MTH_1011 | ATP-dependent 26S | 7.4E−111 |
| | | subunit | | | protease regulatory | |
| | | | | | subunit 8 | |
| MSM0967 | Msp_1408 | HemA | 4.6E−90 | MTH_1012 | glutamyl-tRNA reductase | 3.2E−94 |
| MSM0968 | Msp_1407 | predicted siroheme synthase | 2.4E−45 | MTH_1013 | conserved protein | 1.9E−41 |
| MSM0969 | Msp_1406 | predicted metal-binding | 4.9E−54 | MTH_1014 | conserved protein | 5.6E−58 |
| | | transcription factor | | | | |
| MSM0970 | Msp_0784 | hypothetical protein | 1.3E−21 | NONE | | |
| MSM0971 | Msp_0393 | methyl-coenzyme M reductase, | 7.6E−191 | NONE | methyl coenzyme M | 4.3E−209 |
| | | component A2 | | | reductase system, | |
| | | | | | component A2 | |
| MSM0972 | Msp_1405 | conserved hypothetical protein | 1.3E−46 | MTH_1016 | conserved protein | 5.5E−51 |
| MSM0973 | Msp_1404 | putative GTP cyclohydrolase III | 9.2E−76 | MTH_1017 | conserved protein | 1.3E−88 |
| MSM0974 | Msp_1403 | CofD | 3.6E−90 | MTH_1018 | conserved protein | 8.0E−98 |
| MSM0975 | Msp_1402 | CofE | 3.8E−63 | MTH_1019 | conserved protein | 1.6E−76 |
| MSM0976 | Msp_1398 | PurO | 2.8E−51 | MTH_1020 | conserved protein | 1.0E−51 |
| MSM0977 | Msp_1397 | conserved hypothetical | 3.7E−24 | MTH_1021 | unknown | 3.2E−30 |
| | | membrane-spanning protein | | | | |
| MSM0978 | Msp_1396 | predicted biopolymer transport | 1.5E−77 | MTH_1022 | biopolymer transport | 4.1E−94 |
| | | protein | | | protein | |
| MSM0979 | Msp_1395 | RnhB | 1.6E−48 | MTH_1023 | ribonuclease HII | 9.8E−61 |
| MSM0980 | Msp_1517 | DnaK | 5.3E−16 | MTH_1024 | rod shape-determining | 7.3E−136 |
| | | | | | protein | |
| MSM0981 | NONE | | | MTH_1025 | unknown | 2.6E−51 |
| MSM0982 | Msp_1394 | partially conserved hypothetical | 2.4E−38 | MTH_1027 | CDP-diacylglycerol-serine | 8.2E−41 |
| | | membrane-spanning protein | | | O-phosphatidyltransferase | |
| MSM0983 | Msp_1393 | conserved hypothetical | 8.7E−48 | MTH_1028 | unknown | 1.7E−70 |
| | | membrane-spanning protein | | | | |
| MSM0984 | NONE | | | MTH_1030 | unknown | 1.4E−45 |
| MSM0985 | Msp_1392 | conserved hypothetical protein | 1.1E−29 | MTH_1031 | conserved protein | 6.3E−34 |
| MSM0986 | Msp_0760 | putative bile salt acid hydrolase | 4.3E−110 | NONE | | |
| MSM0987 | Msp_0329 | MfnA | 3.9E−100 | MTH_1116 | glutamate decarboxylase | 6.1E−123 |
| MSM0988 | Msp_0328 | PpsA | 1.7E−273 | MTH_1118 | phosphoenolpyruvate | 2.0E−250 |
| | | | | | synthase | |
| MSM0989 | Msp_0327 | 50S ribosomal protein L10e | 2.8E−58 | MTH_1119 | ribosomal protein L10 | 2.1E−65 |
| MSM0990 | Msp_1000 | predicted ABC-type | 4.7E−40 | MTH_920 | anion permease | 4.2E−37 |
| | | nitrate/sulfonate/bicarbonate | | | | |
| | | transport system, ATB-binding | | | | |
| | | protein | | | | |
| MSM0991 | Msp_1001 | predicted ABC-type | 2.4E−11 | MTH_478 | sulfate transport system | 4.1E−09 |
| | | nitrate/sulfonate/bicarbonate | | | permease protein | |
| | | transport system, permease | | | | |
| | | protein | | | | |
| MSM0992 | Msp_0326 | hypothetical protein | 1.0E−12 | MTH_1121 | unknown | 8.9E−12 |
| MSM0993 | Msp_0601 | partially conserved hypothetical | 3.9E−04 | MTH_1123 | unknown | 1.9E−15 |
| | | protein, predicted GTPase | | | | |
| MSM0994 | Msp_0324 | predicted nucleotidyltransferase | 3.4E−101 | MTH_1126 | conserved protein | 2.7E−90 |
| MSM0995 | Msp_0590 | member of asn/thr-rich large | 8.7E−33 | MTH_716 | cell surface glycoprotein | 1.3E−09 |
| | | protein family | | | (s-layer protein) | |
| MSM0996 | Msp_0983 | member of asn/thr-rich large | 2.6E−26 | MTH_716 | cell surface glycoprotein | 1.1E−09 |
| | | protein family | | | (s-layer protein) | |
| MSM0997 | Msp_0323 | PyrC | 1.1E−97 | MTH_1127 | dihydroorotase | 7.8E−100 |
| MSM0998 | Msp_1447 | EhbK | 1.0E−30 | MTH_1133 | polyferredoxin (MvhB) | 4.4E−145 |
| MSM0999 | Msp_0316 | MvhA | 3.4E−181 | NONE | methyl viologen-reducing | 2.1E−207 |
| | | | | | hydrogenase, alpha | |
| | | | | | subunit | |
| MSM1000 | Msp_0315 | MvhG | 3.2E−128 | NONE | methyl viologen-reducing | 5.5E−138 |
| | | | | | hydrogenase, gamma | |
| | | | | | subunit | |
| MSM1001 | Msp_0314 | MvhD1 | 3.9E−61 | NONE | methyl viologen-reducing | 1.6E−67 |
| | | | | | hydrogenase, delta | |
| | | | | | subunit | |
| MSM1002 | Msp_0312 | conserved hypothetical protein | 1.2E−130 | MTH_1150 | ABC transporter subunit | 3.5E−152 |
| | | | | | Ycf24 | |
| MSM1003 | Msp_0313 | predicted ABC-type transport | 3.2E−82 | MTH_1149 | ABC transporter subunit | 8.0E−98 |
| | | system | | | Ycf16 | |
| MSM1004 | Msp_0311 | conserved hypothetical protein | 1.2E−27 | MTH_1151 | unknown | 9.3E−33 |
| MSM1005 | Msp_0310 | predicted | 4.0E−36 | MTH_1152 | conserved protein | 7.0E−35 |
| | | GTP:adenosylcobinamide- | | | | |
| | | phosphate guanylyltransferase | | | | |
| MSM1006 | Msp_0308 | conserved hypothetical protein | 2.2E−90 | MTH_1153 | conserved protein | 5.2E−165 |
| MSM1007 | Msp_0307 | MtrH | 2.1E−108 | MTH_1156 | N5-methyl- | 2.9E−125 |
| | | | | | tetrahydromethanopterin:coenzyme M | |
| | | | | | methyltransferase, subunit H | |
| MSM1008 | Msp_0306 | MtrG | 5.7E−12 | MTH_1157 | N5-methyl- | 4.2E−21 |
| | | | | | tetrahydromethanopterin:coenzyme M | |
| | | | | | methyltransferase, subunit G | |
| MSM1009 | Msp_0305 | MtrF | 5.5E−07 | MTH_1158 | N5-methyl- | 9.3E−17 |
| | | | | | tetrahydromethanopterin:coenzyme M | |
| | | | | | methyltransferase, subunit F | |
| MSM1010 | Msp_0304 | MtrA | 9.0E−62 | MTH_1159 | N5-methyl- | 9.8E−93 |
| | | | | | tetrahydromethanopterin:coenzyme M | |
| | | | | | methyltransferase, subunit A | |
| MSM1011 | Msp_0303 | MtrB | 1.0E−12 | MTH_1160 | N5-methyl- | 1.7E−31 |
| | | | | | tetrahydromethanopterin:coenzyme M | |
| | | | | | methyltransferase, subunit B | |
| MSM1012 | Msp_0302 | MtrC | 7.6E−49 | MTH_1161 | N5-methyl- | 7.2E−81 |
| | | | | | tetrahydromethanopterin:coenzyme M | |
| | | | | | methyltransferase, subunit C | |
| MSM1013 | Msp_0301 | MtrD | 2.0E−57 | MTH_1162 | N5-methyl- | 1.0E−81 |
| | | | | | tetrahydromethanopterin:coenzyme M | |
| | | | | | methyltransferase, subunit D | |
| MSM1014 | Msp_0300 | MtrE | 9.5E−74 | MTH_1163 | N5-methyl- | 1.5E−121 |
| | | | | | tetrahydromethanopterin:coenzyme M | |
| | | | | | methyltransferase, subunit E | |
| MSM1015 | Msp_0321 | MrtA | 7.6E−207 | NONE | methyl coenzyme M | 1.7E−253 |
| | | | | | reductase I, alpha subunit | |
| MSM1016 | Msp_0320 | MrtG | 6.2E−86 | NONE | methyl coenzyme M | 2.9E−109 |
| | | | | | reductase I, gamma | |
| | | | | | subunit | |
| MSM1017 | Msp_0299 | McrC | 2.8E−67 | NONE | methyl coenzyme M | 2.6E−83 |
| | | | | | reductase I, C protein | |
| MSM1018 | Msp_0319 | MrtD | 7.4E−19 | NONE | methyl coenzyme M | 1.1E−34 |
| | | | | | reductase I, D protein | |
| MSM1019 | Msp_0318 | MrtB | 1.6E−133 | NONE | methyl coenzyme M | 3.4E−177 |
| | | | | | reductase I, beta subunit | |
| MSM1020 | Msp_0298 | predicted Fe—S oxidoreductase | 2.0E−119 | MTH_1170 | conserved protein | 1.7E−136 |
| MSM1021 | Msp_0284 | conserved hypothetical protein | 1.7E−99 | MTH_1180 | conserved protein | 6.7E−117 |
| MSM1022 | Msp_0285 | conserved hypothetical protein | 8.5E−34 | MTH_1181 | unknown | 2.0E−23 |
| MSM1023 | Msp_0973 | ComB2 | 1.3E−44 | MTH_1182 | conserved protein | 2.7E−42 |
| MSM1024 | Msp_0287 | conserved hypothetical | 1.9E−98 | MTH_1183 | pheromone shutdown | 4.4E−58 |
| | | membrane-spanning protein | | | protein TraB | |
| MSM1025 | Msp_0288 | hypothetical protein | 1.5E−20 | MTH_1184 | unknown | 3.0E−20 |
| MSM1026 | NONE | | | MTH_1224 | inosine-5′- | 5.6E−04 |
| | | | | | monophosphate | |
| | | | | | dehydrogenase related | |
| | | | | | protein III | |
| MSM1027 | NONE | | | MTH_1155 | Na+/Ca+ exchanging | 2.1E−42 |
| | | | | | protein related | |
| MSM1028 | Msp_0289 | predicted ATPase | 9.5E−74 | MTH_1186 | conserved protein | 2.0E−85 |
| MSM1029 | Msp_0693 | conserved hypothetical protein | 1.3E−39 | MTH_1187 | conserved protein | 3.2E−23 |
| MSM1030 | Msp_0290 | predicted pyridoxal phosphate- | 1.3E−124 | MTH_1188 | pleiotropic regulatory | 6.1E−123 |
| | | dependent enzyme | | | protein DegT | |
| MSM1031 | Msp_0291 | N2,N2-dimethylguanosine tRNA | 1.1E−109 | NONE | N2,N2-dimethylguanosine | 4.1E−110 |
| | | methyltransferase | | | tRNA methyltransferase | |
| MSM1032 | Msp_0293 | predicted transcriptional regulator | 9.3E−44 | MTH_1193 | transcriptional regulator | 2.9E−52 |
| MSM1033 | Msp_0294 | conserved hypothetical protein | 1.8E−109 | MTH_1196 | conserved protein | 7.7E−116 |
| MSM1034 | Msp_0295 | conserved hypothetical protein | 6.0E−17 | MTH_1197 | conserved protein | 1.1E−22 |
| MSM1035 | Msp_0296 | CofG | 4.2E−96 | MTH_1198 | biotin synthetase related | 6.4E−105 |
| | | | | | protein | |
| MSM1036 | Msp_0297 | predicted methyltransferase | 2.3E−70 | MTH_1200 | met-10+ related protein | 5.7E−72 |
| MSM1037 | Msp_0282 | PsmB | 7.5E−58 | NONE | proteasome, beta subunit | 7.8E−68 |
| MSM1038 | Msp_0281 | predicted exonuclease | 5.4E−245 | MTH_1203 | cleavage and | 3.5E−278 |
| | | | | | polyadenylation specificity | |
| | | | | | factor | |
| MSM1039 | Msp_0280 | PurM | 1.6E−103 | MTH_1204 | phosphoribosylformylglycinamidine | 4.0E−112 |
| | | | | | cyclo-ligase | |
| MSM1040 | Msp_0279 | ComC | 7.6E−104 | MTH_1205 | malate dehydrogenase | 5.7E−104 |
| MSM1041 | Msp_1507 | putative DNA polymerase | 6.8E−167 | MTH_1208 | DNA-dependent DNA | 5.1E−183 |
| | | | | | polymerase family B | |
| | | | | | (PolB1) | |
| MSM1042 | NONE | | | MTH_1211 | conserved protein | 4.0E−71 |
| MSM1043 | Msp_1420 | PyrK | 4.4E−69 | NONE | cytochrome-c3 | 1.6E−74 |
| | | | | | hydrogenase, gamma | |
| | | | | | subunit | |
| MSM1044 | Msp_1421 | PyrD | 7.4E−90 | MTH_1213 | dihydroorotate oxidase | 1.3E−106 |
| MSM1045 | Msp_0220 | predicted glycosyltransferase | 1.9E−12 | MTH_1626 | phosphoserine | 2.4E−05 |
| | | | | | phosphatase | |
| MSM1046 | Msp_1422 | predicted ribosomal biogenesis | 1.2E−89 | MTH_1214 | pre-mRNA splicing protein | 1.4E−88 |
| | | protein | | | PRP31 | |
| MSM1047 | Msp_1423 | FlpA | 5.3E−64 | MTH_1215 | fibrillarin-like pre-rRNA | 2.5E−62 |
| | | | | | processing protein | |
| MSM1048 | Msp_1424 | predicted | 1.9E−43 | MTH_1216 | pantothenate metabolism | 2.3E−52 |
| | | phosphopantothenoylcysteine | | | flavoprotein | |
| | | synthetase/decarboxylase | | | | |
| MSM1049 | Msp_1424 | predicted | 2.0E−55 | MTH_1216 | pantothenate metabolism | 2.2E−54 |
| | | phosphopantothenoylcysteine | | | flavoprotein | |
| | | synthetase/decarboxylase | | | | |
| MSM1050 | Msp_1425 | conserved hypothetical | 4.7E−11 | MTH_1218 | unknown | 3.3E−21 |
| | | membrane-spanning protein | | | | |
| MSM1051 | Msp_1426 | hypothetical membrane-spanning | 3.5E−05 | MTH_1219 | unknown | 9.0E−19 |
| | | protein | | | | |
| MSM1052 | Msp_1427 | PheA | 2.5E−59 | MTH_1220 | chorismate mutase | 1.1E−70 |
| MSM1053 | Msp_1428 | conserved hypothetical protein | 4.4E−60 | MTH_1222 | inosine-5′- | 4.5E−72 |
| | | | | | monophosphate | |
| | | | | | dehydrogenase related | |
| | | | | | protein I | |
| MSM1054 | Msp_1429 | conserved hypothetical protein | 2.2E−74 | MTH_1224 | inosine-5′- | 1.3E−83 |
| | | | | | monophosphate | |
| | | | | | dehydrogenase related | |
| | | | | | protein III | |
| MSM1055 | Msp_1431 | partially conserved hypothetical | 1.9E−36 | MTH_1227 | coenzyme PQQ synthesis | 1.9E−57 |
| | | protein | | | protein III | |
| MSM1056 | Msp_1432 | putative 6-pyruvoyl | 1.4E−38 | MTH_1228 | conserved protein | 4.6E−47 |
| | | tetrahydrobiopterin synthase | | | | |
| MSM1057 | Msp_1433 | conserved hypothetical protein | 2.1E−53 | MTH_1229 | conserved protein | 2.1E−49 |
| MSM1058 | Msp_1434 | conserved hypothetical protein | 5.6E−85 | MTH_1231 | conserved protein | 1.1E−95 |
| MSM1059 | Msp_0945 | predicted RecB family | 1.2E−06 | MTH_1233 | unknown | 1.4E−36 |
| | | exonuclease | | | | |
| MSM1060 | Msp_1436 | EhbQ | 4.9E−61 | MTH_1235 | conserved protein | 1.2E−69 |
| MSM1061 | Msp_1442 | EhbP | 6.3E−22 | MTH_1236 | conserved protein | 1.6E−28 |
| MSM1062 | Msp_1443 | EhbO | 6.1E−79 | NONE | NADH dehydrogenase | 5.8E−111 |
| | | | | | (ubiquinone), subunit 1 | |
| | | | | | related protein | |
| MSM1063 | Msp_1444 | EhbN | 8.0E−141 | NONE | formate hydrogenlyase, | 2.8E−143 |
| | | | | | subunit 5 | |
| MSM1064 | Msp_1445 | EhbM | 1.0E−62 | NONE | formate hydrogenlyase, | 1.6E−67 |
| | | | | | subunit 7 | |
| MSM1065 | Msp_1446 | EhbL | 8.6E−41 | MTH_1240 | ferredoxin-like protein | 3.4E−51 |
| MSM1066 | Msp_1447 | EhbK | 7.7E−72 | MTH_1241 | polyferredoxin | 1.7E−97 |
| MSM1067 | Msp_1448 | EhbJ | 4.5E−12 | MTH_1242 | unknown | 5.5E−19 |
| MSM1068 | Msp_1449 | EhbI | 4.2E−48 | MTH_1243 | conserved protein | 1.0E−49 |
| MSM1069 | Msp_1450 | EhbH | 3.5E−21 | MTH_1244 | conserved protein | 5.0E−25 |
| MSM1070 | Msp_1451 | EhbG | 4.8E−15 | MTH_1245 | unknown | 6.6E−16 |
| MSM1071 | Msp_1452 | EhbF | 1.1E−134 | NONE | NADH dehydrogenase I, | 8.4E−142 |
| | | | | | subunit N | |
| MSM1072 | Msp_1453 | EhbE | 2.0E−32 | MTH_1247 | conserved protein | 4.5E−40 |
| MSM1073 | Msp_1454 | EhbD | 4.1E−18 | MTH_1248 | conserved protein | 9.4E−24 |
| MSM1074 | Msp_1455 | EhbC | 1.4E−10 | MTH_1249 | conserved protein | 1.5E−18 |
| MSM1075 | Msp_1456 | EhbB | 2.2E−10 | MTH_1250 | unknown | 1.1E−13 |
| MSM1076 | Msp_1457 | EhbA | 1.2E−27 | MTH_1251 | conserved protein | 6.8E−37 |
| MSM1077 | Msp_1336 | predicted permease | 2.3E−05 | NONE | | |
| MSM1078 | Msp_1336 | predicted permease | 9.6E−97 | MTH_900 | conserved protein | 3.1E−32 |
| MSM1079 | Msp_1458 | conserved hypothetical | 2.1E−28 | MTH_1252 | conserved protein | 1.6E−35 |
| | | membrane-spanning protein | | | | |
| MSM1080 | NONE | | | MTH_1253 | unknown | 2.5E−48 |
| MSM1081 | Msp_0795 | partially conserved hypothetical | 1.4E−56 | MTH_1634 | transcriptional control | 5.0E−176 |
| | | protein | | | factor (enhancer-binding | |
| | | | | | protein) | |
| MSM1082 | NONE | | | NONE | | |
| MSM1083 | Msp_0202 | conserved hypothetical | 4.5E−35 | MTH_230 | unknown | 1.0E−33 |
| | | membrane-spanning protein | | | | |
| MSM1084 | Msp_1459 | ArgG | 7.4E−138 | MTH_1254 | argininosuccinate | 2.1E−136 |
| | | | | | synthase | |
| MSM1085 | Msp_1240 | AqpM2 | 1.8E−54 | MTH_103 | water channel protein | 1.5E−71 |
| MSM1086 | NONE | | | MTH_101 | unknown | 3.8E−194 |
| MSM1087 | NONE | | | NONE | | |
| MSM1088 | NONE | | | NONE | | |
| MSM1089 | Msp_0506 | hypothetical membrane-spanning | 3.3E−04 | NONE | | |
| | | protein | | | | |
| MSM1090 | Msp_1057 | SfsA | 6.0E−33 | MTH_1521 | sugar fermentation | 3.6E−31 |
| | | | | | stimulation protein | |
| MSM1091 | Msp_1501 | predicted sugar kinase | 3.6E−97 | MTH_1256 | conserved protein | 1.4E−114 |
| MSM1092 | Msp_1502 | formylmethanofuran- | 1.2E−91 | MTH_1259 | formylmethanofuran:tetrahydro- | 1.3E−127 |
| | | tetrahydromethanopterin | | | methanopterin formyltransferase | |
| | | formyltransferase | | | | |
| MSM1093 | Msp_0233 | conserved hypothetical protein | 2.3E−22 | NONE | | |
| MSM1094 | Msp_1503 | conserved hypothetical | 2.8E−81 | MTH_1261 | conserved protein | 7.2E−97 |
| | | membrane-spanning protein | | | | |
| MSM1095 | Msp_0830 | Trk-type potassium transport | 2.6E−62 | MTH_1264 | TRK system potassium | 2.1E−122 |
| | | system, membrane protein | | | uptake protein TrkH | |
| MSM1096 | Msp_0250 | TrkA1 | 3.1E−52 | MTH_1265 | TRK system potassium | 3.6E−79 |
| | | | | | uptake protein TrkA | |
| MSM1097 | Msp_1505 | putative Zn-dependent hydrolase | 2.3E−40 | MTH_1267 | conserved protein | 1.2E−53 |
| MSM1098 | Msp_1418 | putative archaeal holliday junction | 1.4E−38 | MTH_1270 | conserved protein | 1.4E−43 |
| | | resolvase | | | | |
| MSM1099 | Msp_0270 | predicted biotin synthase related | 7.4E−106 | MTH_1279 | conserved protein | 2.3E−75 |
| | | protein | | | | |
| MSM1100 | NONE | | | MTH_627 | unknown | 7.2E−10 |
| MSM1101 | Msp_0269 | GatB | 1.4E−175 | MTH_1280 | PET112-like protein | 3.6E−182 |
| MSM1102 | Msp_0268 | conserved hypothetical protein | 3.4E−78 | MTH_1282 | inosine-5′- | 2.3E−93 |
| | | | | | monophosphate | |
| | | | | | dehydrogenase related | |
| | | | | | protein VI | |
| MSM1103 | Msp_0267 | HisE | 4.8E−31 | MTH_1283 | phosphoribosyl-AMP | 3.0E−34 |
| | | | | | cyclohydrolase homolog | |
| MSM1104 | Msp_1506 | predicted acetyltransferase | 2.6E−11 | MTH_1284 | conserved protein | 3.2E−16 |
| MSM1105 | Msp_1492 | conserved hypothetical protein | 7.0E−62 | MTH_1286 | phosphoribosylaminoimidazole | 1.7E−65 |
| | | | | | carboxylase related | |
| | | | | | protein | |
| MSM1106 | Msp_1497 | HypF | 8.5E−208 | MTH_1287 | transcriptional regulator | 2.3E−219 |
| | | | | | HypF homolog | |
| MSM1107 | Msp_1519 | predicted transcriptional regulator | 6.6E−34 | MTH_1288 | unknown | 1.8E−52 |
| MSM1108 | Msp_1518 | GrpE | 2.1E−44 | MTH_1289 | heat shock protein GrpE | 1.6E−44 |
| MSM1109 | Msp_1517 | DnaK | 8.6E−247 | MTH_1290 | DnaK protein (Hsp70) | 7.7E−251 |
| MSM1110 | Msp_1516 | DnaJ | 3.0E−118 | MTH_1291 | DnaJ protein | 1.0E−122 |
| MSM1111 | Msp_0145 | member of asn/thr-rich large | 5.9E−49 | MTH_716 | cell surface glycoprotein | 7.7E−12 |
| | | protein family | | | (s-layer protein) | |
| MSM1112 | Msp_0762 | member of asn/thr-rich large | 1.6E−40 | MTH_716 | cell surface glycoprotein | 3.3E−11 |
| | | protein family | | | (s-layer protein) | |
| MSM1113 | Msp_0762 | member of asn/thr-rich large | 2.9E−70 | MTH_716 | cell surface glycoprotein | 1.2E−05 |
| | | protein family | | | (s-layer protein) | |
| MSM1114 | Msp_0145 | member of asn/thr-rich large | 1.3E−24 | MTH_716 | cell surface glycoprotein | 3.3E−15 |
| | | protein family | | | (s-layer protein) | |
| MSM1115 | Msp_0017 | conserved hypothetical protein | 2.2E−21 | NONE | | |
| MSM1116 | Msp_1108 | member of asn/thr-rich large | 4.2E−137 | MTH_911 | probable surface protein | 1.5E−12 |
| | | protein family | | | | |
| MSM1117 | Msp_1110 | CobN | 8.5E−304 | MTH_514 | cobalamin biosynthesis | 1.4E−239 |
| | | | | | protein N | |
| MSM1118 | Msp_1494 | hypothetical membrane-spanning | 1.5E−18 | MTH_1294 | unknown | 2.5E−23 |
| | | protein | | | | |
| MSM1119 | Msp_1495 | hypothetical membrane-spanning | 4.1E−25 | MTH_1295 | unknown | 4.8E−36 |
| | | protein | | | | |
| MSM1120 | Msp_1496 | methionine aminopeptidase | 3.4E−53 | MTH_1296 | methionine | 2.8E−86 |
| | | | | | aminopeptidase | |
| MSM1121 | Msp_1305 | FrhB | 3.9E−77 | NONE | coenzyme F420-reducing | 2.1E−97 |
| | | | | | hydrogenase, beta | |
| | | | | | subunit | |
| MSM1122 | Msp_1304 | FrhG | 4.6E−81 | NONE | coenzyme F420-reducing | 2.2E−102 |
| | | | | | hydrogenase, gamma | |
| | | | | | subunit | |
| MSM1123 | Msp_1514 | putative coenzyme F420 | 9.3E−44 | NONE | coenzyme F420-reducing | 4.7E−61 |
| | | hydrogenase, delta subunit-like | | | hydrogenase, delta | |
| | | protein | | | subunit | |
| MSM1124 | Msp_1302 | FrhA | 9.4E−138 | NONE | coenzyme F420-reducing | 8.8E−163 |
| | | | | | hydrogenase, alpha | |
| | | | | | subunit | |
| MSM1125 | Msp_1110 | CobN | 2.3E−10 | MTH_1301 | unknown | 3.8E−11 |
| MSM1126 | Msp_0120 | predicted transcriptional regulator | 3.1E−20 | MTH_1795 | transcriptional regulator | 1.1E−20 |
| MSM1127 | Msp_0121 | predicted cation transport ATPase | 1.2E−162 | MTH_411 | cadmium efflux ATPase | 1.2E−119 |
| MSM1128 | NONE | | | NONE | | |
| MSM1129 | Msp_1523 | conserved hypothetical protein | 2.3E−118 | MTH_1305 | conserved protein | 3.6E−134 |
| MSM1130 | Msp_1028 | conserved hypothetical protein | 4.5E−44 | MTH_1868 | conserved protein | 1.4E−15 |
| MSM1131 | Msp_1524 | conserved hypothetical protein | 1.1E−56 | MTH_1306 | conserved protein | 1.1E−59 |
| MSM1132 | Msp_1525 | ribosome biogenesis protein | 2.3E−15 | MTH_1307 | unknown | 4.0E−16 |
| | | Nop10 | | | | |
| MSM1133 | Msp_1527 | putative translation initiation factor | 3.4E−94 | NONE | translation initiation factor | 3.5E−104 |
| | | 2, alpha subunit (alF-2alpha) | | | eIF-2, alpha subunit | |
| | | (eIF2A) | | | | |
| MSM1134 | Msp_1528 | 30S ribosomal protein S27e | 2.3E−17 | MTH_1309 | ribosomal protein S27 | 8.1E−18 |
| MSM1135 | Msp_1529 | 50S ribosomal protein L44e | 1.6E−41 | MTH_1310 | ribosomal protein L36a | 2.7E−42 |
| MSM1136 | Msp_1530 | partially conserved hypothetical | 1.6E−30 | MTH_1311 | unknown | 2.1E−49 |
| | | protein | | | | |
| MSM1137 | Msp_1531 | DNA polymerase sliding clamp | 1.5E−73 | MTH_1312 | proliferating-cell nuclear | 6.0E−93 |
| | | (PCNA) | | | antigen | |
| MSM1138 | Msp_0580 | predicted glutamine | 5.2E−73 | MTH_787 | cobyric acid synthase | 9.2E−10 |
| | | amidotransferase | | | | |
| MSM1139 | Msp_0581 | predicted UDP-N-acetylmuramyl | 3.6E−90 | MTH_530 | UDP-N-acetylmuramyl | 6.8E−16 |
| | | tripeptide synthase | | | tripeptide synthetase | |
| | | | | | related protein | |
| MSM1140 | Msp_0417 | hypothetical membrane-spanning | 2.7E−04 | NONE | | |
| | | protein | | | | |
| MSM1141 | Msp_1075 | TrpA | 7.3E−44 | NONE | tryptophan synthase, | 6.5E−48 |
| | | | | | subunit alpha | |
| MSM1142 | Msp_1074 | TrpB | 6.4E−123 | NONE | tryptophan synthase, | 1.3E−120 |
| | | | | | beta subunit | |
| MSM1143 | Msp_1072 | TrpC | 1.7E−42 | MTH_1657 | indole-3-glycerol | 1.4E−38 |
| | | | | | phosphate synthase | |
| MSM1144 | Msp_1076 | TrpD | 2.0E−71 | MTH_1661 | anthranilate | 2.3E−68 |
| | | | | | phosphoribosyltransferase | |
| MSM1145 | Msp_1071 | TrpG | 7.4E−51 | MTH_1656 | anthranilate synthase | 1.1E−43 |
| | | | | | component II | |
| MSM1146 | Msp_1070 | TrpE | 6.5E−78 | MTH_1655 | anthranilate synthase | 9.9E−84 |
| | | | | | component I | |
| MSM1147 | NONE | | | NONE | | |
| MSM1148 | NONE | | | MTH_1189 | conserved protein | 8.2E−08 |
| MSM1149 | Msp_0607 | hypothetical membrane-spanning | 6.0E−33 | MTH_1192 | conserved protein | 2.8E−31 |
| | | protein | | | | |
| MSM1150 | Msp_0608 | predicted transcriptional regulator | 9.4E−19 | MTH_1328 | conserved protein | 1.3E−17 |
| MSM1151 | Msp_1247 | PurB | 6.0E−159 | MTH_1537 | adenylosuccinate lyase | 8.4E−174 |
| MSM1152 | Msp_0879 | hypothetical membrane-spanning | 2.8E−04 | MTH_1538 | unknown | 6.4E−25 |
| | | protein | | | | |
| MSM1153 | Msp_0224 | predicted cation transport ATPase | 1.1E−205 | MTH_1535 | heavy-metal transporting | 5.1E−199 |
| | | | | | CPx-type ATPase | |
| MSM1154 | Msp_0200 | predicted metal-dependent | 1.2E−07 | MTH_1534 | aryldialkylphosphatase | 5.0E−89 |
| | | hydrolase | | | related protein | |
| MSM1155 | Msp_0225 | conserved hypothetical protein | 1.4E−40 | MTH_1530 | conserved protein | 1.7E−42 |
| MSM1156 | Msp_0221 | TruD | 6.2E−125 | MTH_1529 | conserved protein | 4.6E−134 |
| MSM1157 | Msp_1512 | hypothetical membrane-spanning | 3.5E−05 | MTH_1526 | conserved protein | 8.9E−04 |
| | | protein | | | | |
| MSM1158 | Msp_1511 | HypE2 | 8.9E−126 | MTH_1525 | hydrogenase | 4.2E−156 |
| | | | | | expression/formation | |
| | | | | | protein HypE related | |
| | | | | | protein | |
| MSM1159 | Msp_1510 | HisH | 3.0E−38 | MTH_1524 | imidazoleglycerol- | 9.1E−58 |
| | | | | | phosphate synthase | |
| MSM1160 | Msp_1461 | predicted nitrogenase | 3.8E−118 | MTH_1522 | nitrogenase alpha chain | 8.9E−131 |
| | | molybdenum-iron protein | | | (NifD) related protein | |
| MSM1161 | Msp_0719 | partially conserved hypothetical | 2.8E−05 | NONE | | |
| | | membrane-spanning protein | | | | |
| MSM1162 | NONE | | | NONE | | |
| MSM1163 | NONE | | | NONE | | |
| MSM1164 | Msp_1463 | predicted GTPase | 1.4E−143 | MTH_1515 | GTP-binding protein | 2.4E−153 |
| MSM1165 | Msp_1472 | predicted phosphohydrolase | 2.2E−67 | MTH_1179 | conserved protein | 9.0E−10 |
| MSM1166 | Msp_1474 | conserved hypothetical membrane- | 1.5E−146 | NONE | | |
| | | spanning protein | | | | |
| MSM1167 | Msp_1464 | CbiE | 6.8E−48 | MTH_1514 | precorrin-6Y methylase | 3.9E−50 |
| MSM1168 | Msp_0590 | member of asn/thr-rich large | 1.7E−16 | MTH_75 | surface protease related | 2.1E−11 |
| | | protein family | | | protein | |
| MSM1169 | NONE | | | NONE | | |
| MSM1170 | Msp_0169 | putative arsenical prump-driving | 5.3E−96 | MTH_1511 | arsenical pump-driving | 6.9E−108 |
| | | ATPase | | | ATPase | |
| MSM1171 | Msp_0170 | NadE | 1.1E−63 | MTH_1510 | NH(3)-dependent NAD+ | 1.3E−60 |
| | | | | | synthetase | |
| MSM1172 | Msp_0171 | LeuS | 0.0E+00 | MTH_1508 | leucyl-tRNA synthetase | 0.0E+00 |
| MSM1173 | Msp_0004 | predicted tRNA(1- | 1.0E−62 | MTH_1414 | protein-L-isoaspartate | 1.4E−77 |
| | | methyladenosine) | | | methyltransferase | |
| | | methyltransferase | | | homolog | |
| MSM1174 | Msp_0309 | HtpX | 1.8E−38 | MTH_569 | heat shock protein X | 2.1E−67 |
| MSM1175 | Msp_0548 | hypothetical membrane-spanning | 6.6E−11 | NONE | | |
| | | protein | | | | |
| MSM1176 | Msp_0413 | RfcS | 2.2E−115 | NONE | replication factor C, small | 3.7E−125 |
| | | | | | subunit | |
| MSM1177 | Msp_0414 | RfcL | 1.1E−113 | NONE | replication factor C, large | 3.8E−123 |
| | | | | | subunit | |
| MSM1178 | Msp_0578 | conserved hypothetical protein | 4.1E−34 | MTH_239 | unknown | 9.7E−38 |
| MSM1179 | Msp_0647 | AroE | 1.8E−72 | MTH_242 | shikimate 5- | 1.2E−71 |
| | | | | | dehydrogenase | |
| MSM1180 | NONE | | | MTH_1189 | conserved protein | 1.6E−08 |
| MSM1181 | Msp_0648 | HisS | 5.1E−114 | MTH_244 | histidyl-tRNA synthetase | 3.8E−130 |
| MSM1182 | Msp_0649 | HisI | 1.6E−39 | MTH_245 | phosphoribosyl-AMP | 1.0E−40 |
| | | | | | cyclohydrolase | |
| MSM1183 | Msp_0650 | predicted ATPase | 1.5E−155 | MTH_246 | twitching mobility (PilT) | 8.0E−185 |
| | | | | | related protein | |
| MSM1184 | Msp_0651 | predicted sugar phosphate | 8.7E−48 | MTH_247 | conserved protein | 4.5E−49 |
| | | isomerase/epimerase or | | | | |
| | | endonuclease | | | | |
| MSM1185 | Msp_1499 | putative methylated-DNA--protein- | 1.3E−12 | MTH_618 | O6-methylguanidine- | 2.8E−15 |
| | | cysteine methyltransferase | | | DNA methyltransferase | |
| MSM1186 | Msp_1489 | predicted potassium transport | 9.9E−111 | NONE | | |
| | | system, membrane component | | | | |
| MSM1187 | Msp_0007 | predicted ERCC4-like helicase | 5.4E−213 | NONE | ATP-dependent RNA | 3.5E−241 |
| | | | | | helicase, eIF-4A family | |
| MSM1188 | Msp_0590 | member of asn/thr-rich large | 1.4E−49 | MTH_716 | cell surface glycoprotein | 6.9E−13 |
| | | protein family | | | (s-layer protein) | |
| MSM1189 | Msp_0017 | conserved hypothetical protein | 1.7E−28 | NONE | | |
| MSM1190 | Msp_1211 | partially conserved hypothetical | 6.7E−128 | MTH_530 | UDP-N-acetylmuramyl | 3.1E−57 |
| | | membrane-spanning protein | | | tripeptide synthetase | |
| | | | | | related protein | |
| MSM1191 | Msp_1212 | predicted UDP-N- | 7.9E−102 | MTH_531 | UDP-N-acetylmuramyl | 1.3E−40 |
| | | acetylmuramoylalanine--D- | | | tripeptide synthetase | |
| | | glutamate ligase | | | related protein | |
| MSM1192 | Msp_0008 | conserved hypothetical protein | 9.1E−124 | MTH_1421 | conserved protein | 5.0E−137 |
| MSM1193 | Msp_0009 | putative single-stranded-DNA- | 9.9E−111 | MTH_1422 | conserved protein | 9.3E−136 |
| | | specific exonuclease | | | | |
| MSM1194 | Msp_0010 | 30S ribosomal protein S15P | 5.3E−48 | MTH_1423 | ribosomal protein S13 | 2.1E−49 |
| | | | | | (E. coli) | |
| MSM1195 | Msp_0011 | putative xanthosine triphosphate | 1.9E−61 | MTH_1424 | conserved protein | 1.2E−62 |
| | | pyrophosphatase | | | | |
| MSM1196 | Msp_0635 | cell division control protein 6-like 2 | 9.7E−06 | NONE | | |
| MSM1197 | NONE | | | NONE | | |
| MSM1198 | Msp_0013 | putative O-sialoglycoprotein | 7.7E−159 | MTH_1425 | O-sialoglycoprotein | 1.9E−174 |
| | | endopeptidase | | | endopeptidase | |
| MSM1199 | Msp_0999 | hypothetical protein | 7.0E−06 | NONE | | |
| MSM1200 | Msp_0012 | predicted | 1.4E−88 | MTH_1426 | conserved protein | 3.4E−99 |
| | | phosphoribosyltransferase | | | | |
| MSM1201 | Msp_0014 | UppP | 6.0E−72 | MTH_1428 | bacitracin resistance | 1.1E−43 |
| | | | | | protein | |
| MSM1202 | Msp_0015 | IlvE | 4.0E−114 | MTH_1430 | branched-chain amino- | 5.2E−110 |
| | | | | | acid aminotransferase | |
| MSM1203 | Msp_0724 | hypothetical membrane-spanning | 6.7E−09 | MTH_470 | conserved protein | 7.9E−05 |
| | | protein | | | | |
| MSM1204 | Msp_0163 | F420-dependent | 4.0E−82 | NONE | coenzyme F420- | 2.2E−102 |
| | | methylenetetrahydromethanopterin | | | dependent N5,N10- | |
| | | dehydrogenase | | | methylene | |
| | | | | | tetrahydromethanopterin | |
| | | | | | dehydrogenase | |
| MSM1205 | Msp_0417 | hypothetical membrane-spanning | 5.3E−04 | MTH_1490 | unknown | 3.5E−17 |
| | | protein | | | | |
| MSM1206 | Msp_0164 | HisB | 2.5E−57 | MTH_1467 | imidazoleglycerol- | 9.7E−54 |
| | | | | | phosphate dehydratase | |
| MSM1207 | NONE | | | MTH_1470 | molybdenum transport | 2.2E−17 |
| | | | | | protein ModA related | |
| | | | | | protein | |
| MSM1208 | Msp_0165 | predicted polysaccharide | 5.0E−116 | MTH_1471 | O-antigen transporter | 3.2E−87 |
| | | biosynthesis protein | | | homolog | |
| MSM1209 | Msp_0540 | predicted multimeric flavodoxin | 6.7E−25 | MTH_1473 | conserved protein | 4.7E−54 |
| MSM1210 | Msp_0925 | predicted arabinose efflux | 7.5E−22 | MTH_195 | efflux pump antibiotic | 2.5E−24 |
| | | permease | | | resistance protein | |
| MSM1211 | Msp_0260 | hypothetical protein | 4.6E−16 | MTH_1626 | phosphoserine | 4.3E−06 |
| | | | | | phosphatase | |
| MSM1212 | NONE | | | NONE | | |
| MSM1213 | Msp_1498 | formaldehyde activating enzyme | 8.3E−162 | MTH_1474 | D-arabino 3-hexulose 6- | 6.3E−169 |
| | | fused to 3-hexulose-6phosphate | | | phosphate formaldehyde | |
| | | synthase | | | lyase related protein | |
| MSM1214 | Msp_1573 | ThrS | 7.3E−202 | MTH_1455 | threonyl-tRNA | 1.3E−225 |
| | | | | | synthetase | |
| MSM1215 | Msp_0162 | CbiA | 1.7E−147 | NONE | cobyrinic acid a,c- | 9.4E−143 |
| | | | | | diamide synthase | |
| MSM1216 | Msp_0166 | conserved hypothetical membrane- | 1.3E−74 | MTH_1461 | conserved protein | 2.1E−67 |
| | | spanning protein | | | | |
| MSM1217 | Msp_0019 | partially conserved hypothetical | 5.0E−45 | MTH_1434 | unknown | 1.3E−55 |
| | | protein | | | | |
| MSM1218 | Msp_0020 | SurE | 1.2E−68 | MTH_1435 | survival protein SurE | 1.5E−73 |
| MSM1219 | NONE | | | NONE | | |
| MSM1220 | NONE | | | MTH_1440 | unknown | 8.6E−14 |
| MSM1221 | Msp_0021 | conserved hypothetical protein | 5.2E−89 | MTH_1441 | conserved protein | 3.4E−106 |
| MSM1222 | Msp_0022 | IlvC | 6.9E−126 | MTH_1442 | ketol-acid | 2.7E−122 |
| | | | | | reductoisomerase | |
| MSM1223 | Msp_0591 | predicted carbonic anhydrase | 8.1E−13 | MTH_1582 | carbonic anhydrase | 3.7E−38 |
| MSM1224 | Msp_0025 | IlvH1 | 1.1E−45 | NONE | acetolactate synthase, | 4.1E−55 |
| | | | | | small subunit | |
| MSM1225 | Msp_0026 | IlvB1 | 6.3E−180 | NONE | acetolactate synthase, | 3.5E−207 |
| | | | | | large subunit | |
| MSM1226 | Msp_0031 | ArgF | 2.3E−102 | MTH_1446 | ornithine | 4.6E−102 |
| | | | | | carbamoyltransferase | |
| MSM1227 | Msp_0030 | PurD | 1.1E−150 | MTH_1445 | glycinamide | 4.2E−147 |
| | | | | | ribonucleotide | |
| | | | | | synthetase | |
| MSM1228 | Msp_0513 | predicted Na+-driven multidrug | 5.6E−108 | MTH_314 | conserved protein | 2.8E−95 |
| | | efflux pump | | | | |
| MSM1229 | Msp_0513 | predicted Na+-driven multidrug | 1.1E−125 | MTH_314 | conserved protein | 3.1E−105 |
| | | efflux pump | | | | |
| MSM1230 | Msp_0512 | predicted transcriptional regulator | 5.3E−25 | MTH_313 | transcriptional regulator | 2.2E−17 |
| MSM1231 | Msp_1574 | ArgS | 1.4E−157 | MTH_1447 | arginyl-tRNA synthetase | 9.3E−175 |
| MSM1232 | Msp_1575 | putative signal peptidase | 3.6E−42 | MTH_1448 | signal peptidase | 2.7E−42 |
| MSM1233 | Msp_1180 | HemL | 5.8E−138 | MTH_228 | glutamate-1- | 2.1E−136 |
| | | | | | semialdehyde | |
| | | | | | aminotransferase | |
| MSM1234 | Msp_1179 | CbiC | 8.2E−68 | MTH_227 | precorrin isomerase | 7.1E−58 |
| MSM1235 | Msp_0093 | predicted flavoprotein | 2.5E−59 | NONE | | |
| MSM1236 | Msp_0135 | AspS | 1.9E−164 | MTH_226 | aspartyl-tRNA | 1.2E−165 |
| | | | | | synthetase | |
| MSM1237 | Msp_1576 | IlvD | 7.2E−195 | MTH_1449 | dihydroxy-acid | 3.4E−177 |
| | | | | | dehydratase | |
| MSM1238 | Msp_0134 | HisD | 2.7E−131 | MTH_225 | histidinol dehydrogenase | 2.7E−138 |
| MSM1239 | Msp_1569 | predicted DNA-binding protein | 2.7E−92 | MTH_1458 | unknown | 5.1E−96 |
| MSM1240 | Msp_1570 | conserved hypothetical protein | 8.9E−23 | MTH_1457 | unknown | 3.0E−24 |
| MSM1241 | Msp_1571 | predicted ATPase | 5.2E−82 | MTH_1456 | chromosome partitioning | 1.9E−73 |
| | | | | | protein Soj | |
| MSM1242 | Msp_1074 | TrpB | 7.2E−37 | NONE | tryptophan synthase, | 1.0E−168 |
| | | | | | beta subunit homolog | |
| MSM1243 | NONE | | | MTH_1477 | unknown | 3.1E−73 |
| MSM1244 | Msp_1491 | predicted metal-dependent | 1.9E−45 | MTH_1478 | conserved protein | 8.9E−28 |
| | | phosphoesterase | | | | |
| MSM1245 | Msp_0198 | AlbA | 2.2E−26 | MTH_1483 | conserved protein | 3.8E−27 |
| MSM1246 | Msp_0199 | LeuA1 | 8.3E−162 | MTH_1481 | isopropylmalate synthase | 2.8E−175 |
| MSM1247 | Msp_0197 | conserved hypothetical membrane- | 2.6E−78 | MTH_1485 | serine/threonine protein | 1.2E−92 |
| | | spanning protein | | | kinase related protein | |
| MSM1248 | Msp_0196 | ABC-type multidrug transport | 4.6E−74 | MTH_1486 | conserved protein | 1.5E−82 |
| | | system, permease protein | | | | |
| MSM1249 | Msp_0195 | ABC-type multidrug transport | 1.6E−94 | MTH_1487 | ABC transporter (ATP- | 5.1E−103 |
| | | system, ATP-binding protein | | | binding | |
| MSM1250 | Msp_0194 | predicted transcriptional regulator | 3.6E−19 | MTH_1488 | unknown | 1.6E−19 |
| MSM1251 | Msp_0651 | predicted sugar phosphate | 7.5E−26 | MTH_1489 | conserved protein | 8.8E−60 |
| | | isomerase/epimerase or | | | | |
| | | endonuclease | | | | |
| MSM1252 | Msp_0191 | MapB | 8.0E−38 | MTH_1493 | cation transporting P- | 1.8E−54 |
| | | | | | type ATPase related | |
| | | | | | protein | |
| MSM1253 | Msp_0181 | GatA | 2.1E−165 | MTH_1496 | amidase | 1.1E−164 |
| MSM1254 | Msp_0174 | predicted cobyric acid synthase | 7.3E−115 | NONE | cobyrinic acid a,c- | 8.9E−115 |
| | | | | | diamide synthase related | |
| | | | | | protein | |
| MSM1255 | NONE | | | NONE | | |
| MSM1256 | Msp_0175 | RibB | 2.5E−59 | MTH_1499 | GTP cyclohydrolase II | 2.8E−63 |
| MSM1257 | Msp_0177 | predicted transcriptional regulator | 1.7E−19 | MTH_1500 | conserved protein | 9.4E−24 |
| MSM1258 | Msp_0180 | TfrA | 2.0E−174 | NONE | succinate | 3.9E−185 |
| | | | | | dehydrogenase, | |
| | | | | | flavoprotein subunit | |
| MSM1259 | Msp_0200 | predicted metal-dependent | 1.0E−115 | MTH_1505 | N-ethylammeline | 9.3E−120 |
| | | hydrolase | | | chlorohydrolase homolog | |
| MSM1260 | Msp_0383 | archaeal histone | 8.8E−16 | MTH_1696 | histone HMtA2 | 8.4E−16 |
| MSM1261 | Msp_0178 | HisG | 1.4E−88 | MTH_1506 | ATP | 1.3E−90 |
| | | | | | phosphoribosyltransferase | |
| MSM1262 | NONE | | | NONE | | |
| MSM1263 | Msp_0003 | PyrB | 8.4E−98 | MTH_1413 | aspartate | 5.1E−96 |
| | | | | | carbamoyltransferase | |
| MSM1264 | Msp_0001 | cell division control protein 6-like 1 | 4.9E−141 | MTH_1412 | Cdc6 related protein | 8.2E−160 |
| MSM1265 | NONE | | | MTH_1410 | unknown | 1.4E−31 |
| MSM1266 | Msp_1588 | CobD | 4.4E−76 | MTH_1409 | cobalamin biosynthesis | 7.6E−54 |
| | | | | | protein B | |
| MSM1267 | Msp_1587 | CbiG | 2.3E−70 | MTH_1408 | cobalamin biosynthesis | 3.0E−50 |
| | | | | | protein G | |
| MSM1268 | Msp_1586 | conserved hypothetical protein | 2.7E−21 | MTH_1407 | conserved protein | 2.6E−28 |
| MSM1269 | NONE | | | NONE | | |
| MSM1270 | Msp_1585 | predicted class II aldolase | 4.7E−40 | MTH_1406 | fuculose-1-phosphate | 4.9E−43 |
| | | | | | aldolase | |
| MSM1271 | Msp_1584 | PolB | 4.5E−131 | MTH_1405 | DNA polymerase delta | 3.6E−156 |
| | | | | | small subunit | |
| MSM1272 | Msp_1583 | hypothetical membrane-spanning | 5.8E−19 | MTH_1404 | unknown | 4.3E−28 |
| | | protein | | | | |
| MSM1273 | Msp_1582 | CbiH | 2.5E−98 | MTH_1403 | precorrin-3 methylase | 1.2E−101 |
| MSM1274 | NONE | | | MTH_1402 | conserved protein | 6.4E−73 |
| MSM1275 | Msp_0962 | hypothetical membrane-spanning | 2.4E−04 | MTH_1401 | unknown | 5.4E−108 |
| | | protein | | | | |
| MSM1276 | Msp_1558 | hypothetical protein | 1.7E−10 | MTH_1400 | unknown | 1.3E−16 |
| MSM1277 | Msp_1559 | conserved hypothetical membrane- | 8.0E−38 | MTH_1399 | unknown | 2.0E−46 |
| | | spanning protein | | | | |
| MSM1278 | Msp_0757 | predicted ATPase | 4.3E−101 | NONE | | |
| MSM1279 | Msp_1562 | conserved hypothetical protein | 1.5E−50 | MTH_1398 | conserved protein | 2.3E−52 |
| MSM1280 | Msp_1561 | conserved hypothetical protein | 5.0E−52 | MTH_1397 | conserved protein | 1.2E−25 |
| MSM1281 | Msp_1563 | CbiX | 7.5E−42 | MTH_1397 | conserved protein | 8.6E−30 |
| MSM1282 | Msp_0590 | member of asn/thr-rich large | 3.1E−13 | MTH_716 | cell surface glycoprotein | 2.7E−05 |
| | | protein family | | | (s-layer protein) | |
| MSM1283 | Msp_1564 | ThiL | 6.8E−48 | MTH_1396 | thiamine monphosphate | 3.1E−57 |
| | | | | | kinase | |
| MSM1284 | Msp_1565 | predicted pyruvate-formate lyase- | 1.5E−66 | MTH_1395 | pyruvate formate-lyase | 3.5E−81 |
| | | activating enzyme | | | activating enzyme | |
| | | | | | related protein | |
| MSM1285 | Msp_0615 | partially conserved hypothetical | 6.8E−05 | NONE | | |
| | | membrane-spanning protein | | | | |
| MSM1286 | Msp_1479 | predicted 3-octaprenyl-4- | 5.7E−147 | MTH_1394 | conserved protein | 3.5E−152 |
| | | hydroxybenzoate carboxy-lyase | | | | |
| MSM1287 | Msp_1480 | PurE | 6.4E−68 | MTH_1393 | phosphoribosylaminoimidazole | 1.9E−80 |
| | | | | | carboxylase | |
| MSM1288 | NONE | | | NONE | | |
| MSM1289 | Msp_1168 | CobS | 6.5E−04 | NONE | | |
| MSM1290 | Msp_0054 | predicted glycosyltransferase | 1.4E−33 | MTH_374 | dolichyl-phosphate | 7.5E−31 |
| | | | | | mannose synthase | |
| | | | | | related protein | |
| MSM1291 | NONE | | | NONE | | |
| MSM1292 | Msp_0920 | predicted transcriptional accessory | 9.5E−232 | NONE | translation initiation | 2.1E−04 |
| | | protein | | | factor eIF-2, alpha | |
| | | | | | subunit | |
| MSM1293 | Msp_0965 | predicted nitroreductase | 3.3E−16 | MTH_120 | NADPH-oxidoreductase | 2.1E−33 |
| MSM1294 | Msp_1481 | conserved hypothetical membrane- | 3.4E−124 | MTH_1392 | dolichyl-phosphate | 5.8E−150 |
| | | spanning protein | | | mannoosyltransferase | |
| | | | | | related protein | |
| MSM1295 | Msp_1482 | conserved hypothetical membrane- | 7.0E−94 | MTH_1391 | conserved protein | 3.8E−114 |
| | | spanning protein | | | | |
| MSM1296 | Msp_1483 | RibH | 2.0E−50 | MTH_1390 | riboflavin synthase beta | 1.4E−54 |
| | | | | | subunit | |
| MSM1297 | Msp_0219 | conserved hypothetical protein | 3.0E−70 | NONE | | |
| MSM1298 | Msp_1484 | LeuB | 3.8E−109 | MTH_1388 | 3-isopropylmalate | 3.2E−103 |
| | | | | | dehydrogenase | |
| MSM1299 | Msp_1485 | LeuD1 | 3.1E−43 | NONE | 3-isopropylmalate | 3.3E−60 |
| | | | | | dehydratase, LeuC | |
| | | | | | subunit | |
| MSM1300 | Msp_1486 | LeuC1 | 1.3E−165 | NONE | 3-isopropylmalate | 1.7E−175 |
| | | | | | dehydratase, LeuD | |
| | | | | | subunit | |
| MSM1301 | NONE | | | NONE | | |
| MSM1302 | NONE | | | NONE | | |
| MSM1303 | Msp_0214 | predicted UDP-N-acetyl-D- | 2.3E−143 | MTH_836 | UDP-N-acetyl-D- | 2.8E−79 |
| | | mannosaminuronate | | | mannosaminuronic acid | |
| | | dehydrogenase | | | dehydrogenase | |
| MSM1304 | Msp_1116 | predicted dTDP-4- | 9.6E−42 | MTH_1792 | dTDP-4- | 1.9E−73 |
| | | dehydrorhamnose reductase | | | dehydrorhamnose | |
| | | | | | reductase | |
| MSM1305 | Msp_0762 | member of asn/thr-rich large | 5.3E−36 | MTH_716 | cell surface glycoprotein | 2.2E−12 |
| | | protein family | | | (s-layer protein) | |
| MSM1306 | Msp_0590 | member of asn/thr-rich large | 3.5E−45 | MTH_716 | cell surface glycoprotein | 1.8E−07 |
| | | protein family | | | (s-layer protein) | |
| MSM1307 | Msp_1102 | predicted dTDP-glucose | 4.1E−41 | MTH_1791 | glucose-1-phosphate | 1.4E−123 |
| | | pyrophosphorylase | | | thymidylyltransferase | |
| MSM1308 | Msp_0539 | predicted dTDP-4- | 1.9E−68 | NONE | dTDP-4- | 5.4E−60 |
| | | dehydrorhamnose 3,5-epimerase | | | dehydrorhamnose 3,5- | |
| | | | | | epimerase | |
| MSM1309 | Msp_1114 | predicted dTDP-D-glucose 4,6- | 4.5E−106 | NONE | dTDP-glucose 4,6- | 3.0E−137 |
| | | dehydratase | | | dehydratase | |
| MSM1310 | Msp_0212 | predicted glycosyltransferase | 1.8E−54 | MTH_884 | teichoic acid biosynthesis | 7.1E−10 |
| | | | | | related protein | |
| MSM1311 | Msp_0496 | predicted glycosyltransferase | 2.8E−34 | MTH_136 | dolichyl-phosphate | 2.2E−05 |
| | | | | | mannose synthase | |
| MSM1312 | Msp_0500 | predicted glycosyltransferase | 4.8E−79 | MTH_172 | conserved protein | 6.5E−19 |
| MSM1313 | Msp_0492 | predicted glycosyltransferase | 6.1E−57 | MTH_338 | LPS biosynthesis RfbU | 2.9E−07 |
| | | | | | related protein | |
| MSM1314 | NONE | | | NONE | | |
| MSM1315 | NONE | | | NONE | | |
| MSM1316 | Msp_0495 | predicted glycosyltransferase | 2.3E−33 | MTH_884 | teichoic acid biosynthesis | 8.9E−09 |
| | | | | | related protein | |
| MSM1317 | Msp_0500 | predicted glycosyltransferase | 2.9E−07 | NONE | | |
| MSM1318 | Msp_0927 | hypothetical protein | 2.1E−30 | NONE | | |
| MSM1319 | Msp_0928 | hypothetical protein | 3.0E−31 | NONE | | |
| MSM1320 | Msp_0492 | predicted glycosyltransferase | 4.1E−58 | NONE | | |
| MSM1321 | Msp_0500 | predicted glycosyltransferase | 4.4E−76 | MTH_172 | conserved protein | 9.5E−17 |
| MSM1322 | Msp_0492 | predicted glycosyltransferase | 6.5E−62 | MTH_338 | LPS biosynthesis RfbU | 9.6E−12 |
| | | | | | related protein | |
| MSM1323 | Msp_0495 | predicted glycosyltransferase | 5.3E−34 | MTH_884 | teichoic acid biosynthesis | 2.0E−08 |
| | | | | | related protein | |
| MSM1324 | Msp_0215 | predicted glycosyltransferase | 1.0E−32 | MTH_884 | teichoic acid biosynthesis | 1.5E−08 |
| | | | | | related protein | |
| MSM1325 | Msp_0204 | predicted ABC-type | 1.2E−64 | MTH_1092 | putative membrane | 6.6E−06 |
| | | polysaccharide/polyol phosphate | | | protein | |
| | | export system, permease protein | | | | |
| MSM1326 | Msp_0205 | predicted ABC-type | 3.7E−79 | MTH_1370 | ABC transporter (ATP- | 2.0E−16 |
| | | polysaccharide/polyol phosphate | | | binding protein) | |
| | | export system, ATP-binding protein | | | | |
| MSM1327 | NONE | | | MTH_361 | teichoic acid biosynthesis | 2.4E−17 |
| | | | | | protein RodC related | |
| | | | | | protein | |
| MSM1328 | Msp_0212 | predicted glycosyltransferase | 2.9E−26 | MTH_884 | teichoic acid biosynthesis | 2.0E−12 |
| | | | | | related protein | |
| MSM1329 | Msp_0206 | predicted glycosyltransferase | 5.2E−82 | MTH_172 | conserved protein | 2.5E−46 |
| MSM1330 | Msp_0207 | predicted glycosyltransferase | 9.1E−69 | MTH_172 | conserved protein | 1.1E−20 |
| MSM1331 | Msp_0208 | predicted bacterial sugar | 9.0E−117 | NONE | | |
| | | transferase | | | | |
| MSM1332 | Msp_1487 | predicted ssDNA-binding protein | 6.2E−157 | MTH_1385 | replication factor A | 7.8E−152 |
| | | | | | related protein | |
| MSM1333 | Msp_1488 | RadA | 6.9E−142 | MTH_1383 | DNA repair protein RadA | 6.4E−144 |
| MSM1334 | Msp_1477 | predicted permease | 1.4E−56 | MTH_1382 | conserved protein | 1.2E−57 |
| MSM1335 | NONE | | | NONE | | |
| MSM1336 | Msp_1476 | HdrA1 | 6.9E−277 | NONE | heterodisulfide | 2.0E−298 |
| | | | | | reductase, subunit A | |
| MSM1337 | Msp_1475 | GlyA | 5.9E−145 | MTH_1380 | serine | 6.5E−151 |
| | | | | | hydroxymethyltransferase | |
| MSM1338 | Msp_1473 | predicted flavoprotein | 3.4E−53 | MTH_1379 | conserved protein | 5.0E−73 |
| | | | | | (contains ferredoxin | |
| | | | | | domain) | |
| MSM1339 | Msp_1471 | conserved hypothetical protein | 2.5E−11 | MTH_1377 | conserved protein | 9.7E−22 |
| MSM1340 | Msp_1470 | S-adenosylmethionine synthetase | 2.2E−138 | MTH_1376 | conserved protein | 3.7E−148 |
| MSM1341 | Msp_1468 | IleS | 0.0E+00 | MTH_1375 | isoleucyl-tRNA | 0.0E+00 |
| | | | | | synthetase | |
| MSM1342 | Msp_1467 | PurL | 5.9E−239 | MTH_1374 | phosphoribosylformylglycinamidine | 4.4E−255 |
| | | | | | synthase II | |
| MSM1343 | NONE | | | MTH_1369 | molybdenum cofactor | 2.5E−110 |
| | | | | | biosynthesis MoeA | |
| MSM1344 | Msp_1466 | predicted membrane-associated | 1.4E−81 | MTH_1368 | conserved protein | 3.4E−99 |
| | | Zn-dependent protease | | | | |
| MSM1345 | NONE | | | NONE | | |
| MSM1346 | Msp_0822 | hypothetical protein | 1.6E−06 | NONE | | |
| MSM1347 | NONE | | | NONE | | |
| MSM1348 | Msp_0789 | rubrerythrin | 2.7E−04 | MTH_1351 | conserved protein | 4.2E−37 |
| MSM1349 | Msp_0787 | FprA | 2.9E−136 | MTH_1350 | flavoprotein AI | 2.7E−152 |
| MSM1350 | Msp_0061 | conserved hypothetical protein | 5.4E−32 | MTH_1349 | conserved protein | 3.1E−48 |
| MSM1351 | Msp_0038 | CbiL | 1.1E−58 | MTH_1348 | precorrin-2 | 9.8E−61 |
| | | | | | methyltransferase | |
| MSM1352 | Msp_0036 | putative ATP-dependent helicase | 1.1E−175 | MTH_1347 | probable ATP-dependent | 3.4E−212 |
| | | | | | helicase | |
| MSM1353 | Msp_1532 | hypothetical membrane-spanning | 1.6E−08 | MTH_1313 | unknown | 9.0E−13 |
| | | protein | | | | |
| MSM1354 | Msp_1533 | RpoM1 | 4.7E−33 | MTH_1314 | transcription elongation | 4.8E−36 |
| | | | | | factor TFIIS | |
| MSM1355 | Msp_1534 | putative ADP-ribose | 4.9E−38 | MTH_1315 | mutator MutT protein | 1.1E−34 |
| | | pyrophosphatase | | | | |
| MSM1356 | Msp_1535 | RpoL | 2.1E−14 | NONE | DNA-dependent RNA | 5.5E−19 |
| | | | | | polymerase, subunit L | |
| MSM1357 | Msp_1536 | predicted RNA-binding protein | 2.6E−32 | MTH_1318 | conserved protein | 1.6E−46 |
| MSM1358 | Msp_1537 | predicted diphthamide synthase, | 6.1E−95 | MTH_1319 | conserved protein | 1.1E−109 |
| | | subunit DPH2 | | | | |
| MSM1359 | Msp_1538 | putative adenine | 5.0E−52 | MTH_1320 | adenine | 2.2E−54 |
| | | phosphoribosyltransferase | | | phosphoribosyltransferase | |
| MSM1360 | Msp_1539 | signal recognition particle, 54 kDa | 2.0E−151 | MTH_1321 | signal recognition particle | 5.8E−159 |
| | | protein | | | protein SRP54 | |
| MSM1361 | Msp_1541 | predicted pseudouridylate synthase | 4.0E−82 | MTH_1322 | conserved protein | 1.0E−104 |
| MSM1362 | NONE | | | MTH_809 | molybdenum cofactor | 2.2E−47 |
| | | | | | biosynthesis protein | |
| | | | | | MoaC | |
| MSM1363 | Msp_0229 | SecG | 2.2E−12 | NONE | | |
| MSM1364 | Msp_0032 | HisF | 1.6E−112 | MTH_1343 | imidazoleglycerol- | 3.7E−109 |
| | | | | | phosphate synthase | |
| | | | | | (cyclase) | |
| MSM1365 | Msp_0034 | putative 3-methyladenine DNA | 2.1E−37 | MTH_1342 | 8-oxoguanine DNA | 1.1E−68 |
| | | glycosylase/8-oxoguanine DNA | | | glycosylase | |
| | | glycosylase | | | | |
| MSM1366 | NONE | | | MTH_758 | S-D-lactoylglutathione | 7.2E−26 |
| | | | | | methylglyoxal lyase | |
| MSM1367 | Msp_0035 | predicted peptidyl-prolyl cis-trans | 2.3E−63 | MTH_1338 | peptidyl-prolyl cis-trans | 1.9E−57 |
| | | isomerase 1 | | | isomerase B | |
| MSM1368 | Msp_0037 | ArgD | 6.6E−121 | MTH_1337 | N-acetylornithine | 8.1E−121 |
| | | | | | aminotransferase | |
| MSM1369 | Msp_0006 | predicted NUDIX-related protein | 4.5E−12 | MTH_1336 | mutator MutT protein | 1.0E−17 |
| | | | | | homolog | |
| MSM1370 | Msp_0715 | conserved hypothetical membrane- | 9.6E−97 | NONE | | |
| | | spanning protein | | | | |
| MSM1371 | Msp_1578 | LysA | 2.9E−152 | MTH_1335 | diaminopimelate | 2.3E−155 |
| | | | | | decarboxylase | |
| MSM1372 | Msp_1579 | DapF | 1.3E−74 | MTH_1334 | diaminopimelate | 2.8E−86 |
| | | | | | epimerase | |
| MSM1373 | Msp_1545 | conserved hypothetical protein | 3.2E−50 | MTH_1329 | methyltransferase related | 4.1E−46 |
| | | | | | protein | |
| MSM1374 | Msp_1544 | KsgA | 1.6E−62 | MTH_1326 | dimethyladenosine | 1.3E−56 |
| | | | | | transferase | |
| MSM1375 | NONE | | | MTH_1325 | conserved protein | 2.9E−61 |
| MSM1376 | Msp_1543 | conserved hypothetical protein | 5.1E−20 | MTH_1324 | conserved protein | 2.1E−28 |
| MSM1377 | Msp_1542 | 50S ribosomal protein L21e | 3.3E−32 | MTH_1323 | ribosomal protein L21 | 2.7E−35 |
| MSM1378 | Msp_0981 | conserved hypothetical protein | 7.4E−19 | NONE | | |
| MSM1379 | Msp_0967 | putative NADP-dependent alcohol | 1.4E−24 | NONE | | |
| | | dehydrogenase | | | | |
| MSM1380 | Msp_0967 | putative NADP-dependent alcohol | 4.6E−74 | NONE | | |
| | | dehydrogenase | | | | |
| MSM1381 | Msp_0967 | putative NADP-dependent alcohol | 2.2E−11 | NONE | | |
| | | dehydrogenase | | | | |
| MSM1382 | Msp_0504 | conserved hypothetical membrane- | 2.7E−53 | NONE | | |
| | | spanning protein | | | | |
| MSM1383 | Msp_0254 | anaerobic ribonucleotide- | 1.6E−307 | MTH_1539 | anaerobic | 9.9E−306 |
| | | triphosphate reductase | | | ribonucleoside- | |
| | | | | | triphosphate reductase | |
| MSM1384 | Msp_0255 | PolC | 3.9E−290 | MTH_1536 | conserved protein | 0.0E+00 |
| MSM1385 | Msp_0113 | conserved hypothetical protein | 7.7E−16 | MTH_1626 | phosphoserine | 2.3E−09 |
| | | | | | phosphatase | |
| MSM1386 | NONE | | | NONE | | |
| MSM1387 | Msp_0249 | LysS | 4.8E−205 | MTH_1542 | conserved protein | 2.6E−202 |
| MSM1388 | Msp_0251 | ThiC2 | 1.0E−156 | MTH_1543 | thiamine biosynthesis | 5.3E−172 |
| | | | | | protein | |
| MSM1389 | Msp_0252 | predicted ribokinase | 1.3E−78 | MTH_1544 | ribokinase | 3.8E−91 |
| MSM1390 | Msp_0248 | conserved hypothetical protein | 2.5E−50 | MTH_1545 | conserved protein | 1.5E−55 |
| MSM1391 | Msp_0247 | predicted sugar phosphate | 1.2E−52 | MTH_1546 | conserved protein | 1.3E−51 |
| | | isomerase | | | | |
| MSM1392 | NONE | | | NONE | nitrate assimilation | 4.4E−58 |
| | | | | | protein, narQ | |
| MSM1393 | NONE | | | NONE | | |
| MSM1394 | Msp_0355 | conserved hypothetical membrane- | 1.5E−04 | NONE | | |
| | | spanning protein | | | | |
| MSM1395 | Msp_0340 | PstB | 3.1E−27 | MTH_605 | ABC transporter | 3.2E−30 |
| MSM1396 | NONE | | | MTH_1345 | conserved protein | 4.7E−22 |
| MSM1397 | Msp_0432 | member of asn/thr-rich large protein | 7.3E−30 | MTH_911 | probable surface protein | 3.0E−12 |
| | | family | | | | |
| MSM1398 | Msp_0762 | member of asn/thr-rich large protein | 4.2E−21 | MTH_716 | cell surface glycoprotein | 2.4E−10 |
| | | family | | | (s-layer protein) | |
| MSM1399 | Msp_0911 | member of asn/thr-rich large protein | 5.8E−13 | MTH_716 | cell surface glycoprotein | 4.7E−13 |
| | | family | | | (s-layer protein) | |
| MSM1400 | Msp_0615 | partially conserved hypothetical | 5.3E−05 | MTH_672 | unknown | 1.6E−04 |
| | | membrane-spanning protein | | | | |
| MSM1401 | Msp_1106 | conserved hypothetical membrane- | 5.9E−42 | MTH_671 | unknown | 1.9E−48 |
| | | spanning protein | | | | |
| MSM1402 | Msp_1107 | conserved hypothetical membrane- | 4.2E−16 | MTH_670 | unknown | 2.4E−11 |
| | | spanning protein | | | | |
| MSM1403 | NONE | | | NONE | | |
| MSM1404 | Msp_0243 | FwdB | 5.2E−23 | NONE | formate dehydrogenase, | 1.9E−153 |
| | | | | | alpha subunit homolog | |
| MSM1405 | Msp_0639 | FdhB | 5.0E−84 | NONE | formate dehydrogenase, | 7.8E−84 |
| | | | | | beta subunit related | |
| | | | | | protein FlpB | |
| MSM1406 | Msp_0384 | predicted Fe—S oxidoreductase | 2.7E−19 | MTH_1550 | molybdenum cofactor | 2.6E−99 |
| | | | | | biosynthesis MoaA | |
| MSM1407 | Msp_0488 | predicted allosteric regulator of | 9.7E−04 | MTH_1551 | molybdopterin-guanine | 2.3E−36 |
| | | homoserine dehydrogenase | | | dinucleotide biosynthesis | |
| | | | | | protein B related | |
| MSM1408 | Msp_0147 | ferredoxin | 7.5E−10 | NONE | tungsten | 8.3E−48 |
| | | | | | formylmethanofuran | |
| | | | | | dehydrogenase, subunit H | |
| MSM1409 | Msp_1447 | EhbK | 6.0E−18 | NONE | tungsten | 3.1E−97 |
| | | | | | formylmethanofuran | |
| | | | | | dehydrogenase, subunit F | |
| MSM1410 | Msp_0241 | FwdG | 1.8E−22 | NONE | tungsten | 2.7E−19 |
| | | | | | formylmethanofuran | |
| | | | | | dehydrogenase, subunit G | |
| MSM1411 | Msp_0242 | FwdD | 5.4E−39 | NONE | tungsten | 6.9E−21 |
| | | | | | formylmethanofuran | |
| | | | | | dehydrogenase, subunit D | |
| MSM1412 | Msp_0243 | FwdB | 1.6E−156 | NONE | tungsten | 5.3E−117 |
| | | | | | formylmethanofuran | |
| | | | | | dehydrogenase, subunit B | |
| MSM1413 | Msp_0244 | FwdA | 6.4E−203 | NONE | tungsten | 1.7E−182 |
| | | | | | formylmethanofuran | |
| | | | | | dehydrogenase, subunit A | |
| MSM1414 | Msp_0245 | FwdC | 1.9E−66 | NONE | tungsten | 2.9E−52 |
| | | | | | formylmethanofuran | |
| | | | | | dehydrogenase, subunit C | |
| MSM1415 | Msp_0246 | hypothetical protein | 3.9E−13 | MTH_1568 | unknown | 1.1E−08 |
| MSM1416 | Msp_0246 | hypothetical protein | 6.8E−09 | MTH_1568 | unknown | 1.6E−05 |
| MSM1417 | Msp_0235 | conserved hypothetical membrane- | 2.9E−150 | MTH_1569 | conserved protein | 6.5E−151 |
| | | spanning protein | | | | |
| MSM1418 | Msp_0234 | GlnA | 3.8E−157 | MTH_1570 | glutamine synthetase | 4.7E−164 |
| MSM1419 | Msp_0017 | conserved hypothetical protein | 1.7E−28 | NONE | | |
| MSM1420 | Msp_0128 | predicted helicase | 5.7E−11 | MTH_511 | DNA helicase II | 1.5E−13 |
| MSM1421 | Msp_1566 | conserved hypothetical membrane- | 4.4E−92 | NONE | | |
| | | spanning protein | | | | |
| MSM1422 | Msp_1568 | conserved hypothetical membrane- | 3.5E−67 | NONE | | |
| | | spanning protein | | | | |
| MSM1423 | Msp_0721 | partially conserved hypothetical | 5.9E−42 | NONE | | |
| | | protein | | | | |
| MSM1424 | Msp_0720 | polyphosphate kinase | 2.4E−258 | NONE | | |
| MSM1425 | Msp_0871 | 30S ribosomal protein S13P | 7.7E−56 | MTH_34 | ribosomal protein S18 | 2.9E−54 |
| | | | | | (E. coli) | |
| MSM1426 | Msp_0870 | 30S ribosomal protein S4P | 6.5E−59 | MTH_35 | ribosomal protein S9 | 4.4E−65 |
| | | | | | (E. coli) | |
| MSM1427 | Msp_0869 | 30S ribosomal protein S11P | 2.5E−59 | MTH_36 | ribosomal protein S14 | 2.9E−61 |
| | | | | | (E. coli) | |
| MSM1428 | Msp_0868 | RpoD | 6.3E−61 | NONE | DNA-dependent RNA | 9.1E−74 |
| | | | | | polymerase, subunit D | |
| MSM1429 | Msp_0867 | 50S ribosomal protein L18e | 1.1E−33 | MTH_38 | ribosomal protein L18 | 5.5E−35 |
| | | | | | (E. coli) | |
| MSM1430 | Msp_0866 | 50S ribosomal protein L13P | 1.3E−51 | MTH_39 | ribosomal protein S16 | 7.1E−58 |
| | | | | | (E. coli) | |
| MSM1431 | Msp_0865 | 30S ribosomal protein S9P | 2.9E−56 | MTH_39 | ribosomal protein S16 | 1.3E−56 |
| | | | | | (E. coli) | |
| MSM1432 | Msp_0864 | RpoN | 9.4E−19 | NONE | DNA-dependent RNA | 1.3E−24 |
| | | | | | polymerase, subunit N | |
| MSM1433 | Msp_0863 | RpoK | 6.9E−16 | NONE | DNA-dependent RNA | 2.4E−18 |
| | | | | | polymerase, subunit K | |
| MSM1434 | NONE | | | NONE | | |
| MSM1435 | Msp_0862 | enolase | 2.2E−113 | MTH_43 | enolase | 3.0E−121 |
| MSM1436 | Msp_0861 | ferredoxin | 3.0E−15 | MTH_1106 | ferredoxin | 6.2E−20 |
| MSM1437 | Msp_0860 | ribosomal protein S2P | 3.9E−84 | MTH_44 | ribosomal protein Sa | 5.5E−83 |
| | | | | | (E. coli) | |
| MSM1438 | Msp_0859 | conserved hypothetical protein | 1.9E−59 | MTH_45 | conserved protein | 5.1E−64 |
| MSM1439 | Msp_0858 | putative mevalonate kinase | 2.1E−60 | MTH_46 | mevalonate kinase | 4.6E−63 |
| MSM1440 | Msp_0857 | predicted archaeal kinase | 9.2E−60 | MTH_47 | conserved protein | 3.6E−70 |
| MSM1441 | Msp_0856 | isopentenyl-diphosphate delta- | 6.2E−118 | MTH_48 | conserved protein | 4.1E−117 |
| | | isomerase | | | | |
| MSM1442 | Msp_0855 | predicted hydrolase | 8.3E−178 | MTH_49 | conserved protein | 8.6E−188 |
| MSM1443 | Msp_0854 | IdsA | 1.3E−90 | MTH_50 | bifunctional short chain | 4.1E−94 |
| | | | | | isoprenyl diphosphate | |
| | | | | | synthase | |
| MSM1444 | NONE | | | NONE | | |
| MSM1445 | Msp_1125 | predicted transcriptional regulator | 1.4E−38 | MTH_1454 | conserved protein | 2.9E−45 |
| MSM1446 | Msp_1126 | putative hydroxylamine reductase | 1.8E−152 | MTH_1453 | 6Fe—6S prismane- | 3.6E−173 |
| | | | | | containing protein | |
| MSM1447 | Msp_0002 | conserved hypothetical protein | 1.1E−31 | MTH_1452 | unknown | 2.3E−36 |
| MSM1448 | Msp_1545 | conserved hypothetical protein | 1.9E−08 | MTH_146 | precorrin-8W | 1.7E−05 |
| | | | | | decarboxylase | |
| MSM1449 | Msp_0219 | conserved hypothetical protein | 7.9E−04 | MTH_83 | O-linked GlcNAc | 9.2E−05 |
| | | | | | transferase | |
| MSM1450 | Msp_0524 | predicted oxidoreductase | 8.4E−25 | MTH_907 | conserved protein | 6.8E−08 |
| MSM1451 | Msp_0039 | predicted glycosyltransferase | 2.2E−06 | MTH_83 | O-linked GlcNAc | 3.2E−10 |
| | | | | | transferase | |
| MSM1452 | Msp_0923 | GltX | 1.1E−184 | MTH_51 | glutamyl-tRNA | 8.5E−181 |
| | | | | | synthetase | |
| MSM1453 | NONE | | | NONE | | |
| MSM1454 | Msp_0226 | hypothetical protein | 9.5E−14 | NONE | heterodisulfide | 6.6E−06 |
| | | | | | reductase, subunit C | |
| MSM1455 | Msp_0924 | predicted | 3.8E−166 | MTH_52 | aspartate | 6.6E−158 |
| | | aspartate/tyrosine/aromatic | | | aminotransferase related | |
| | | aminotransferase | | | protein | |
| MSM1456 | NONE | | | NONE | | |
| MSM1457 | NONE | | | NONE | | |
| MSM1458 | NONE | | | NONE | | |
| MSM1459 | Msp_0925 | predicted arabinose efflux | 7.3E−115 | MTH_195 | efflux pump antibiotic | 7.7E−93 |
| | | permease | | | resistance protein | |
| MSM1460 | Msp_1447 | EhbK | 1.8E−33 | MTH_1133 | polyferredoxin (MvhB) | 5.8E−143 |
| MSM1461 | Msp_0638 | MvhD2 | 1.3E−53 | NONE | methyl viologen-reducing | 2.7E−58 |
| | | | | | hydrogenase, delta | |
| | | | | | subunit homolog FlpD | |
| MSM1462 | Msp_0639 | FdhB | 1.2E−119 | NONE | formate dehydrogenase, | 1.9E−135 |
| | | | | | beta subunit related | |
| | | | | | protein FlpB | |
| MSM1463 | Msp_0640 | FdhA | 4.1E−50 | NONE | formate dehydrogenase, | 2.0E−39 |
| | | | | | alpha subunit related | |
| | | | | | protein FlpC | |
| MSM1464 | NONE | | | MTH_1141 | conserved protein (FlpE) | 1.2E−18 |
| MSM1465 | Msp_0925 | predicted arabinose efflux | 1.3E−115 | MTH_195 | efflux pump antibiotic | 9.5E−95 |
| | | permease | | | resistance protein | |
| MSM1466 | NONE | | | NONE | | |
| MSM1467 | NONE | | | NONE | | |
| MSM1468 | Msp_0986 | PurA | 7.6E−136 | MTH_615 | adenylosuccinate | 9.4E−143 |
| | | | | | synthetase | |
| MSM1469 | Msp_1164 | predicted ABC-type | 2.4E−91 | MTH_924 | molybdate-binding | 5.9E−06 |
| | | nitrate/sulfonate/bicarbonate | | | periplasmic protein | |
| | | transport system, periplasmic | | | | |
| | | solute-binding protein | | | | |
| MSM1470 | NONE | | | NONE | | |
| MSM1471 | Msp_0919 | predicted acyl-CoA synthetase | 2.3E−237 | NONE | succinyl-CoA synthetase, | 2.5E−07 |
| | | | | | alpha subunit | |
| MSM1472 | NONE | | | MTH_752 | conserved protein | 3.7E−77 |
| MSM1473 | Msp_0575 | predicted metal-dependent | 2.9E−79 | MTH_751 | conserved protein | 9.4E−72 |
| | | hydrolase | | | | |
| MSM1474 | Msp_0579 | AroC | 7.2E−124 | MTH_748 | chorismate synthase | 4.7E−125 |
| MSM1475 | Msp_0497 | putative endonuclease III | 1.0E−14 | MTH_746 | endonuclease III related | 2.1E−51 |
| | | | | | protein | |
| MSM1476 | Msp_0416 | HemB | 6.2E−102 | MTH_744 | porphobilinogen | 3.6E−102 |
| | | | | | synthase | |
| MSM1477 | Msp_0428 | predicted ATP:dephospho-CoA | 1.7E−58 | MTH_743 | conserved protein | 5.9E−70 |
| | | triphosphoribosyl transferase | | | | |
| MSM1478 | Msp_0429 | PheS | 2.6E−165 | MTH_742 | phenylalanyl-tRNA | 5.5E−170 |
| | | | | | synthetase | |
| MSM1479 | NONE | | | MTH_212 | exodeoxyribonuclease | 2.4E−73 |
| MSM1480 | Msp_1260 | predicted hydrolase | 1.5E−59 | MTH_209 | conserved protein | 1.1E−77 |
| MSM1481 | Msp_1281 | conserved hypothetical protein | 6.5E−59 | MTH_208 | DNA-dependent DNA | 2.0E−69 |
| | | | | | polymerase family B | |
| | | | | | (PolB2) | |
| MSM1482 | NONE | | | NONE | | |
| MSM1483 | Msp_0195 | ABC-type multidrug transport | 2.0E−41 | MTH_1093 | ABC transporter (ATP- | 1.4E−54 |
| | | system, ATP-binding protein | | | binding | |
| MSM1484 | Msp_0196 | ABC-type multidrug transport | 8.1E−29 | MTH_1486 | conserved protein | 1.0E−19 |
| | | system, permease protein | | | | |
| MSM1485 | Msp_0440 | member of asn/thr-rich large protein | 3.3E−06 | NONE | | |
| | | family | | | | |
| MSM1486 | Msp_1280 | 30S ribosomal protein S8e | 6.6E−34 | MTH_207 | ribosomal protein S8 | 1.5E−41 |
| MSM1487 | NONE | | | MTH_199 | unknown | 9.6E−31 |
| MSM1488 | Msp_0977 | conserved hypothetical protein | 3.1E−27 | MTH_200 | cobalamin biosynthesis | 3.0E−50 |
| | | | | | protein M related protein | |
| MSM1489 | Msp_0474 | hypothetical protein | 1.2E−09 | MTH_1346 | unknown | 1.3E−177 |
| MSM1490 | Msp_0474 | hypothetical protein | 7.1E−06 | MTH_201 | unknown | 4.9E−11 |
| MSM1491 | Msp_0474 | hypothetical protein | 9.8E−08 | MTH_1346 | unknown | 1.3E−159 |
| MSM1492 | Msp_1279 | HypE1 | 1.0E−122 | MTH_205 | hydrogenase | 3.2E−126 |
| | | | | | expression/formation | |
| | | | | | protein HypE | |
| MSM1493 | Msp_1278 | conserved hypothetical membrane- | 1.3E−21 | MTH_204 | conserved protein | 4.3E−19 |
| | | spanning protein | | | | |
| MSM1494 | NONE | | | NONE | | |
| MSM1495 | Msp_1089 | predicted nuclease | 1.8E−40 | MTH_494 | thermonuclease | 8.5E−39 |
| | | | | | precursor | |
| MSM1496 | Msp_0024 | hypothetical protein | 4.5E−67 | NONE | | |
| MSM1497 | NONE | | | MTH_1785 | coenzyme PQQ | 6.4E−57 |
| | | | | | synthesis protein | |
| MSM1498 | Msp_1228 | predicted helicase | 2.1E−131 | NONE | ATP-dependent RNA | 3.8E−114 |
| | | | | | helicase, eIF-4A family | |
| MSM1499 | Msp_1188 | predicted transcriptional regulator | 8.1E−61 | MTH_163 | conserved protein | 2.5E−62 |
| MSM1500 | Msp_1189 | RecJ | 1.5E−114 | MTH_164 | single-stranded DNA | 1.1E−116 |
| | | | | | exonuclease RecJ | |
| | | | | | related protein | |
| MSM1501 | Msp_1190 | signal recognition particle, 19 kDa | 4.0E−20 | MTH_165 | signal recognition particle | 9.3E−17 |
| | | protein | | | 19 kDa protein | |
| MSM1502 | Msp_0223 | predicted UDP-galactopyranose | 3.6E−65 | MTH_344 | UDP-galactopyranose | 2.4E−80 |
| | | mutase | | | mutase | |
| MSM1503 | Msp_0215 | predicted glycosyltransferase | 4.0E−39 | MTH_884 | teichoic acid biosynthesis | 2.4E−06 |
| | | | | | related protein | |
| MSM1504 | Msp_1191 | HemD | 2.2E−49 | MTH_166 | uroporphyrinogen III | 1.1E−52 |
| | | | | | synthase | |
| MSM1505 | NONE | | | NONE | | |
| MSM1506 | NONE | | | NONE | | |
| MSM1507 | Msp_0215 | predicted glycosyltransferase | 5.6E−34 | MTH_884 | teichoic acid biosynthesis | 7.4E−10 |
| | | | | | related protein | |
| MSM1508 | NONE | | | NONE | | |
| MSM1509 | NONE | | | NONE | | |
| MSM1510 | NONE | | | NONE | | |
| MSM1511 | NONE | | | NONE | | |
| MSM1512 | Msp_0060 | putative lipooligosaccharide | 7.0E−62 | NONE | | |
| | | cholinephosphotransferase | | | | |
| MSM1513 | Msp_0662 | putative aspartate aminotransferase | 2.7E−37 | MTH_1601 | aspartate | 1.9E−41 |
| | | | | | aminotransferase | |
| MSM1514 | Msp_1333 | predicted dehydrogenase | 1.3E−06 | NONE | 3-chlorobenzoate-3,4- | 8.7E−09 |
| | | | | | dioxygenase | |
| | | | | | dyhydrogenase related | |
| | | | | | protein | |
| MSM1515 | Msp_0060 | putative lipooligosaccharide | 1.1E−24 | NONE | | |
| | | cholinephosphotransferase | | | | |
| MSM1516 | Msp_1326 | HisC | 1.7E−26 | MTH_1587 | histidinol-phosphate | 5.5E−22 |
| | | | | | aminotransferase | |
| MSM1517 | NONE | | | MTH_1495 | omithine cyclodeaminase | 1.2E−15 |
| MSM1518 | Msp_0017 | conserved hypothetical protein | 1.2E−11 | NONE | | |
| MSM1519 | NONE | | | NONE | | |
| MSM1520 | NONE | | | NONE | | |
| MSM1521 | NONE | | | NONE | | |
| MSM1522 | NONE | | | NONE | | |
| MSM1523 | NONE | | | NONE | | |
| MSM1524 | NONE | | | NONE | | |
| MSM1525 | NONE | | | NONE | | |
| MSM1526 | Msp_0772 | hypothetical membrane-spanning | 2.3E−15 | MTH_252 | conserved protein | 7.1E−19 |
| | | protein | | | | |
| MSM1527 | NONE | | | NONE | | |
| MSM1528 | Msp_0608 | predicted transcriptional regulator | 1.9E−04 | MTH_700 | conserved protein | 1.1E−04 |
| MSM1529 | NONE | | | NONE | | |
| MSM1530 | NONE | | | NONE | | |
| MSM1531 | Msp_0691 | predicted Na+-dependent | 1.3E−131 | NONE | | |
| | | transporter | | | | |
| MSM1532 | Msp_0691 | predicted Na+-dependent | 2.0E−137 | NONE | | |
| | | transporter | | | | |
| MSM1533 | Msp_1465 | member of asn/thr-rich large protein | 7.2E−12 | MTH_1074 | putative membrane | 3.7E−06 |
| | | family | | | protein | |
| MSM1534 | Msp_0590 | member of asn/thr-rich large protein | 2.0E−24 | MTH_1074 | putative membrane | 3.0E−123 |
| | | family | | | protein | |
| MSM1535 | Msp_1114 | predicted dTDP-D-glucose 4,6- | 1.3E−10 | NONE | dTDP-glucose 4,6- | 1.2E−06 |
| | | dehydratase | | | dehydratase | |
| MSM1536 | Msp_0290 | predicted pyridoxal phosphate- | 6.9E−71 | MTH_1188 | pleiotropic regulatory | 6.6E−71 |
| | | dependent enzyme | | | protein DegT | |
| MSM1537 | Msp_0310 | predicted | 4.2E−04 | NONE | | |
| | | GTP:adenosylcobinamide- | | | | |
| | | phosphate guanylyltransferase | | | | |
| MSM1538 | Msp_1202 | predicted acetyltransferase | 1.9E−08 | NONE | N-terminal | 3.5E−06 |
| | | | | | acetyltransferase | |
| | | | | | complex, subunit ARD1 | |
| MSM1539 | NONE | | | NONE | | |
| MSM1540 | NONE | | | MTH_368 | glycerol-3-phosphate | 6.5E−48 |
| | | | | | dehydrogenase (NAD) | |
| MSM1541 | NONE | | | NONE | | |
| MSM1542 | Msp_0310 | predicted | 4.6E−06 | MTH_1152 | conserved protein | 1.4E−04 |
| | | GTP:adenosylcobinamide- | | | | |
| | | phosphate guanylyltransferase | | | | |
| MSM1543 | NONE | | | NONE | | |
| MSM1544 | Msp_0060 | putative lipooligosaccharide | 3.9E−22 | NONE | | |
| | | cholinephosphotransferase | | | | |
| MSM1545 | Msp_0495 | predicted glycosyltransferase | 1.3E−31 | MTH_136 | dolichyl-phosphate | 1.4E−08 |
| | | | | | mannose synthase | |
| MSM1546 | NONE | | | NONE | | |
| MSM1547 | Msp_1195 | PurC | 3.9E−77 | MTH_170 | phosphoribosylaminoimidazolesuccino- | 6.8E−69 |
| | | | | | carboxamide synthase | |
| MSM1548 | Msp_1194 | predicted | 1.2E−25 | MTH_169 | conserved protein | 4.5E−24 |
| | | phosphoribosylformylglycinamidine | | | | |
| | | synthase | | | | |
| MSM1549 | Msp_1193 | PurQ | 2.4E−75 | MTH_168 | phosphoribosylformylglycinamidine | 6.8E−85 |
| | | | | | synthase I | |
| MSM1550 | Msp_1192 | CobA | 6.2E−86 | MTH_167 | S-adenosyl-L-methionine | 7.1E−90 |
| | | | | | uroporphyrinogen | |
| | | | | | methyltransferase | |
| MSM1551 | Msp_1196 | GlmS | 1.5E−201 | MTH_171 | glutamine-fructose-6- | 1.5E−208 |
| | | | | | phosphate transaminase | |
| MSM1552 | NONE | | | NONE | | |
| MSM1553 | NONE | | | NONE | | |
| MSM1554 | Msp_0141 | member of asn/thr-rich large protein | 1.1E−09 | NONE | | |
| | | family | | | | |
| MSM1555 | Msp_0076 | conserved hypothetical protein | 3.5E−60 | MTH_175 | conserved protein | 4.7E−77 |
| MSM1556 | Msp_1344 | conserved hypothetical membrane- | 6.5E−75 | NONE | | |
| | | spanning protein | | | | |
| MSM1557 | Msp_0520 | predicted queuine/archaeosine | 5.0E−219 | MTH_176 | tRNA-guanine | 1.2E−206 |
| | | tRNA-ribosyltransferase | | | transglycosylase | |
| MSM1558 | NONE | | | MTH_1329 | methyltransferase related | 3.1E−04 |
| | | | | | protein | |
| MSM1559 | Msp_0063 | predicted polysaccharide | 9.5E−74 | MTH_379 | O-antigen transporter | 1.7E−72 |
| | | biosynthesis protein | | | related protein | |
| MSM1560 | Msp_0448 | predicted polysaccharide | 1.3E−78 | MTH_379 | O-antigen transporter | 4.9E−75 |
| | | biosynthesis protein | | | related protein | |
| MSM1561 | Msp_0117 | predicted 3-hydroxy-3- | 3.6E−145 | MTH_792 | 3-hydroxy-3- | 3.4E−145 |
| | | methylglutaryl CoA synthase | | | methylglutaryl-CoA- | |
| | | | | | synthase | |
| MSM1562 | Msp_0116 | predicted thiolase | 2.1E−156 | MTH_793 | lipid-transfer protein | 3.5E−168 |
| | | | | | (sterol or nonspecific) | |
| MSM1563 | NONE | | | NONE | | |
| MSM1564 | Msp_0087 | CbiT | 4.6E−05 | NONE | | |
| MSM1565 | Msp_1226 | CobQ | 9.4E−154 | MTH_787 | cobyric acid synthase | 1.1E−162 |
| MSM1566 | Msp_0233 | conserved hypothetical protein | 2.3E−22 | NONE | | |
| MSM1567 | Msp_0762 | member of asn/thr-rich large protein | 7.2E−35 | MTH_1485 | serine/threonine protein | 5.1E−13 |
| | | family | | | kinase related protein | |
| MSM1568 | NONE | | | NONE | | |
| MSM1569 | Msp_1227 | predicted ATP-dependent protease | 2.4E−226 | MTH_785 | ATP-dependent protease | 9.0E−241 |
| | | | | | LA | |
| MSM1570 | Msp_0557 | hypothetical protein | 1.1E−127 | MTH_530 | UDP-N-acetylmuramyl | 2.6E−25 |
| | | | | | tripeptide synthetase | |
| | | | | | related protein | |
| MSM1571 | NONE | | | NONE | | |
| MSM1572 | Msp_0683 | hypothetical protein | 4.9E−61 | NONE | | |
| MSM1573 | NONE | | | NONE | | |
| MSM1574 | Msp_0797 | predicted nitroreductase | 6.3E−10 | MTH_120 | NADPH-oxidoreductase | 4.2E−11 |
| MSM1575 | Msp_1055 | hypothetical membrane-spanning | 7.8E−04 | MTH_521 | unknown | 8.2E−05 |
| | | protein | | | | |
| MSM1576 | NONE | | | NONE | | |
| MSM1577 | Msp_1229 | ribose-phosphate | 1.2E−84 | MTH_784 | ribose-phosphate | 1.0E−88 |
| | | pyrophosphokinase | | | pyrophosphokinase | |
| MSM1578 | NONE | | | NONE | | |
| MSM1579 | Msp_0573 | UvrB | 1.2E−247 | MTH_442 | excinuclease ABC | 1.2E−261 |
| | | | | | subunit B | |
| MSM1580 | NONE | | | NONE | | |
| MSM1581 | Msp_0574 | UvrA | 0.0E+00 | MTH_443 | excinuclease ABC | 0.0E+00 |
| | | | | | subunit A | |
| MSM1582 | Msp_0603 | conserved hypothetical membrane- | 5.6E−85 | MTH_465 | unknown | 4.8E−84 |
| | | spanning protein | | | | |
| MSM1583 | Msp_1178 | predicted helicase | 7.4E−193 | MTH_656 | ATP-dependent RNA | 2.1E−232 |
| | | | | | helicase related protein | |
| MSM1584 | Msp_1119 | conserved hypothetical protein | 1.0E−37 | MTH_641 | conserved protein | 2.9E−29 |
| MSM1585 | Msp_0983 | member of asn/thr-rich large protein | 5.5E−38 | MTH_911 | probable surface protein | 9.9E−06 |
| | | family | | | | |
| MSM1586 | Msp_0713 | member of asn/thr-rich large protein | 1.8E−52 | MTH_911 | probable surface protein | 3.7E−14 |
| | | family | | | | |
| MSM1587 | Msp_0590 | member of asn/thr-rich large protein | 6.0E−44 | MTH_716 | cell surface glycoprotein | 1.2E−06 |
| | | family | | | (s-layer protein) | |
| MSM1588 | NONE | | | NONE | | |
| MSM1589 | NONE | | | NONE | | |
| MSM1590 | Msp_0619 | member of asn/thr-rich large protein | 2.5E−48 | MTH_716 | cell surface glycoprotein | 1.3E−07 |
| | | family | | | (s-layer protein) | |
| MSM1591 | Msp_1118 | conserved hypothetical protein | 1.0E−37 | MTH_639 | conserved protein | 5.6E−42 |
| MSM1592 | Msp_0205 | predicted ABC-type | 9.8E−72 | MTH_1370 | ABC transporter (ATP- | 1.5E−20 |
| | | polysaccharide/polyol phosphate | | | binding protein) | |
| | | export system, ATP-binding protein | | | | |
| MSM1593 | Msp_0204 | predicted ABC-type | 1.3E−53 | MTH_1092 | putative membrane | 5.7E−11 |
| | | polysaccharide/polyol phosphate | | | protein | |
| | | export system, permease protein | | | | |
| MSM1594 | Msp_0442 | predicted glycosyltransferase | 4.4E−60 | MTH_884 | teichoic acid biosynthesis | 1.5E−07 |
| | | | | | related protein | |
| MSM1595 | Msp_0929 | predicted helicase | 6.7E−04 | NONE | | |
| MSM1596 | Msp_0017 | conserved hypothetical protein | 1.7E−28 | NONE | | |
| MSM1597 | NONE | | | NONE | | |
| MSM1598 | NONE | | | NONE | | |
| MSM1599 | NONE | | | NONE | | |
| MSM1600 | NONE | | | NONE | | |
| MSM1601 | Msp_0692 | hypothetical membrane-spanning | 1.3E−07 | NONE | | |
| | | protein | | | | |
| MSM1602 | Msp_0220 | predicted glycosyltransferase | 6.9E−20 | MTH_361 | teichoic acid biosynthesis | 1.7E−04 |
| | | | | | protein RodC related | |
| | | | | | protein | |
| MSM1603 | NONE | | | MTH_637 | conserved protein | 1.1E−20 |
| MSM1604 | Msp_1101 | predicted UDP-glucose | 1.2E−103 | MTH_634 | UTP--glucose-1- | 7.6E−109 |
| | | pyrophosphorylase | | | phosphate | |
| | | | | | uridylyltransferase | |
| MSM1605 | NONE | | | NONE | | |
| MSM1606 | Msp_0612 | predicted arylsulfatase regulatory | 4.8E−102 | MTH_114 | arylsulfatase regulatory | 1.9E−64 |
| | | protein | | | protein | |
| MSM1607 | Msp_1060 | hypothetical protein | 2.4E−13 | MTH_121 | unknown | 1.2E−05 |
| MSM1608 | Msp_1350 | putative oxidoreductase | 5.9E−97 | MTH_907 | conserved protein | 8.1E−50 |
| MSM1609 | NONE | | | MTH_924 | molybdate-binding | 6.6E−23 |
| | | | | | periplasmic protein | |
| MSM1610 | Msp_0342 | PstC | 1.1E−15 | MTH_921 | anion transport system | 6.4E−25 |
| | | | | | permease protein | |
| MSM1611 | Msp_1000 | predicted ABC-type | 1.7E−28 | MTH_920 | anion permease | 2.4E−34 |
| | | nitrate/sulfonate/bicarbonate | | | | |
| | | transport system, ATB-binding | | | | |
| | | protein | | | | |
| MSM1612 | Msp_0210 | predicted UDP-glucose 6- | 6.3E−93 | MTH_836 | UDP-N-acetyl-D- | 5.4E−24 |
| | | dehydrogenase | | | mannosaminuronic acid | |
| | | | | | dehydrogenase | |
| MSM1613 | NONE | | | NONE | | |
| MSM1614 | Msp_0394 | predicted transcriptional regulator | 1.3E−74 | MTH_126 | inosine-5′- | 2.1E−97 |
| | | | | | monophosphate | |
| | | | | | dehydrogenase related | |
| | | | | | protein VII | |
| MSM1615 | Msp_0395 | putative deoxyhypusine synthase | 7.4E−106 | MTH_127 | deoxyhypusine synthase | 4.6E−95 |
| MSM1616 | Msp_0396 | hypothetical membrane-spanning | 4.0E−27 | MTH_128 | unknown | 6.2E−27 |
| | | protein | | | | |
| MSM1617 | Msp_0397 | PyrF | 1.9E−66 | MTH_129 | orotidine 5′ | 4.3E−67 |
| | | | | | monophosphate | |
| | | | | | decarboxylase | |
| MSM1618 | Msp_0398 | CbiM1 | 6.0E−72 | MTH_130 | cobalamin biosynthesis | 9.5E−79 |
| | | | | | protein M | |
| MSM1619 | Msp_0399 | CbiN | 3.0E−31 | MTH_131 | cobalt transport protein N | 7.2E−26 |
| MSM1620 | Msp_0400 | CbiQ1 | 3.0E−38 | MTH_132 | cobalt transport protein Q | 3.4E−42 |
| MSM1621 | Msp_0401 | CbiO1 | 6.0E−88 | MTH_133 | cobalt transport ATP- | 9.3E−88 |
| | | | | | binding protein O | |
| MSM1622 | Msp_1239 | RibC | 6.9E−55 | MTH_134 | riboflavin synthase | 2.3E−61 |
| MSM1623 | Msp_0541 | predicted glycosyltransferase | 2.1E−46 | MTH_136 | dolichyl-phosphate | 6.1E−52 |
| | | | | | mannose synthase | |
| MSM1624 | Msp_0542 | hypothetical membrane-spanning | 9.4E−19 | MTH_137 | unknown | 1.2E−18 |
| | | protein | | | | |
| MSM1625 | Msp_1044 | TfrB | 3.2E−34 | MTH_1850 | fumarate reductase | 7.6E−33 |
| MSM1626 | Msp_1044 | TfrB | 3.0E−07 | MTH_140 | conserved protein | 4.8E−107 |
| MSM1627 | Msp_0989 | predicted glycosyltransferase | 9.5E−11 | MTH_377 | dolichyl-phosphate | 2.0E−11 |
| | | | | | mannose synthase | |
| | | | | | related protein | |
| MSM1628 | Msp_0430 | conserved hypothetical protein | 1.9E−75 | MTH_141 | conserved protein | 7.0E−99 |
| MSM1629 | Msp_0431 | GuaB | 2.1E−163 | MTH_142 | inosine-5′- | 1.5E−174 |
| | | | | | monophosphate | |
| | | | | | dehydrogenase | |
| MSM1630 | Msp_1253 | 50S ribosomal protein L37Ae | 6.0E−33 | MTH_681 | ribosomal protein L37a | 1.1E−36 |
| MSM1631 | NONE | | | NONE | | |
| MSM1632 | Msp_1254 | partially conserved hypothetical | 1.0E−21 | MTH_680 | conserved protein | 1.4E−15 |
| | | protein | | | | |
| MSM1633 | Msp_1255 | conserved hypothetical protein | 1.0E−12 | MTH_679 | unknown | 5.3E−14 |
| MSM1634 | Msp_1256 | partially conserved hypothetical | 2.5E−27 | MTH_678 | conserved protein | 2.1E−35 |
| | | protein | | | | |
| MSM1635 | NONE | | | MTH_677 | unknown | 1.7E−10 |
| MSM1636 | Msp_1257 | conserved hypothetical protein | 2.6E−39 | MTH_669 | phosphoribosylformimino- | 1.3E−58 |
| | | | | | 5-aminoimidazole | |
| | | | | | carboxamide ribotide | |
| | | | | | isomerase related protein | |
| MSM1637 | Msp_0173 | hypothetical membrane-spanning | 9.9E−08 | NONE | | |
| | | protein | | | | |
| MSM1638 | Msp_1259 | hypothetical membrane-spanning | 1.6E−09 | MTH_667 | unknown | 3.0E−11 |
| | | protein | | | | |
| MSM1639 | Msp_0519 | predicted Co/Zn/Cd cation | 4.1E−16 | MTH_1893 | cation efflux system | 3.7E−17 |
| | | transporter | | | protein (zinc/cadmium) | |
| MSM1640 | Msp_0482 | hypothetical membrane-spanning | 1.8E−38 | NONE | | |
| | | protein | | | | |
| MSM1641 | NONE | | | NONE | | |
| MSM1642 | NONE | | | NONE | | |
| MSM1643 | NONE | | | NONE | | |
| MSM1644 | NONE | | | NONE | | |
| MSM1645 | NONE | | | NONE | | |
| MSM1646 | NONE | | | NONE | | |
| MSM1647 | NONE | | | NONE | | |
| MSM1648 | NONE | | | NONE | | |
| MSM1649 | NONE | | | NONE | | |
| MSM1650 | Msp_0260 | hypothetical protein | 7.9E−04 | NONE | | |
| MSM1651 | NONE | | | NONE | | |
| MSM1652 | NONE | | | NONE | | |
| MSM1653 | NONE | | | NONE | | |
| MSM1654 | NONE | | | NONE | | |
| MSM1655 | Msp_1059 | hypothetical protein | 1.3E−05 | NONE | | |
| MSM1656 | NONE | | | NONE | | |
| MSM1657 | Msp_0793 | hypothetical protein | 4.9E−06 | NONE | | |
| MSM1658 | NONE | | | NONE | | |
| MSM1659 | NONE | | | NONE | | |
| MSM1660 | NONE | | | NONE | | |
| MSM1661 | NONE | | | NONE | | |
| MSM1662 | NONE | | | NONE | | |
| MSM1663 | NONE | | | NONE | | |
| MSM1664 | NONE | | | NONE | | |
| MSM1665 | NONE | | | NONE | | |
| MSM1666 | Msp_0946 | conserved hypothetical protein | 1.2E−05 | NONE | | |
| MSM1667 | NONE | | | NONE | | |
| MSM1668 | NONE | | | NONE | | |
| MSM1669 | NONE | | | NONE | | |
| MSM1670 | Msp_0113 | conserved hypothetical protein | 1.8E−04 | NONE | | |
| MSM1671 | NONE | | | NONE | | |
| MSM1672 | NONE | | | NONE | | |
| MSM1673 | Msp_0474 | hypothetical protein | 4.6E−04 | NONE | | |
| MSM1674 | Msp_0822 | hypothetical protein | 2.5E−04 | NONE | | |
| MSM1675 | NONE | | | NONE | | |
| MSM1676 | NONE | | | NONE | | |
| MSM1677 | NONE | | | NONE | | |
| MSM1678 | NONE | | | NONE | | |
| MSM1679 | NONE | | | NONE | | |
| MSM1680 | NONE | | | NONE | | |
| MSM1681 | NONE | | | NONE | | |
| MSM1682 | NONE | | | NONE | | |
| MSM1683 | NONE | | | NONE | | |
| MSM1684 | Msp_0912 | member of asn/thr-rich large protein | 2.1E−06 | MTH_412 | conserved protein | 4.7E−04 |
| | | family | | | | |
| MSM1685 | NONE | | | NONE | | |
| MSM1686 | NONE | | | NONE | | |
| MSM1687 | Msp_0658 | hypothetical membrane-spanning | 8.1E−07 | MTH_1459 | unknown | 3.6E−07 |
| | | protein | | | | |
| MSM1688 | NONE | | | NONE | | |
| MSM1689 | NONE | | | NONE | | |
| MSM1690 | NONE | | | NONE | | |
| MSM1691 | Msp_1039 | partially conserved hypothetical | 1.5E−07 | MTH_357 | conserved protein | 5.3E−08 |
| | | membrane-spanning protein | | | | |
| MSM1692 | NONE | | | NONE | | |
| MSM1693 | Msp_1258 | predicted ribokinase | 6.9E−39 | MTH_668 | unknown | 1.8E−20 |
| MSM1694 | Msp_0929 | predicted helicase | 3.6E−193 | MTH_487 | DNA helicase related | 4.9E−304 |
| | | | | | protein | |
| MSM1695 | Msp_0572 | UvrC | 6.3E−164 | MTH_441 | excinuclease ABC | 5.6E−161 |
| | | | | | subunit C | |
| MSM1696 | Msp_1548 | hypothetical protein | 1.7E−08 | NONE | | |
| MSM1697 | NONE | | | NONE | | |
| MSM1698 | Msp_0439 | methyl-coenzyme M reductase, | 2.7E−147 | NONE | methyl coenzyme M | 5.4E−179 |
| | | component A2-like protein | | | reductase system, | |
| | | | | | component A2 homolog | |
| MSM1699 | Msp_0438 | predicted universal stress protein | 2.1E−14 | MTH_153 | conserved protein | 5.4E−21 |
| MSM1700 | Msp_1061 | hypothetical protein | 7.3E−12 | MTH_278 | ferredoxin | 1.4E−20 |
| MSM1701 | Msp_1062 | predicted dehydrogenase | 4.0E−130 | MTH_277 | bacteriochlorophyll | 8.8E−147 |
| | | | | | synthase 43 kDa subunit | |
| MSM1702 | Msp_1088 | ExoB | 7.9E−102 | MTH_631 | UDP-glucose 4- | 3.5E−97 |
| | | | | | epimerase | |
| MSM1703 | NONE | | | MTH_647 | unknown | 5.0E−25 |
| MSM1704 | Msp_1122 | PurF | 1.4E−143 | MTH_646 | amidophosphoribosyltransferase | 1.2E−156 |
| MSM1705 | Msp_1121 | predicted peptidase | 2.4E−100 | MTH_645 | collagenase | 3.7E−100 |
| MSM1706 | Msp_1513 | hypothetical membrane-spanning | 2.9E−24 | NONE | | |
| | | protein | | | | |
| MSM1707 | Msp_1120 | NifH | 2.6E−96 | MTH_643 | nitrogenase NifH subunit | 5.5E−99 |
| MSM1708 | NONE | | | NONE | | |
| MSM1709 | Msp_0440 | member of asn/thr-rich large protein | 1.3E−35 | MTH_716 | cell surface glycoprotein | 2.4E−04 |
| | | family | | | (s-layer protein) | |
| MSM1710 | Msp_1277 | SerS | 1.9E−187 | MTH_1455 | threonyl-tRNA | 5.3E−06 |
| | | | | | synthetase | |
| MSM1711 | Msp_0725 | hypothetical protein | 1.0E−08 | NONE | | |
| MSM1712 | Msp_0852 | predicted ferritin | 8.4E−50 | MTH_158 | ferritin like protein (RsgA) | 2.3E−59 |
| MSM1713 | Msp_1008 | predicted regulatory protein | 5.4E−32 | MTH_162 | unknown | 1.5E−41 |
| MSM1714 | Msp_1040 | coenzyme F390 synthetase II | 6.3E−164 | MTH_161 | coenzyme F390 | 3.7E−164 |
| | | | | | synthetase III | |
| MSM1715 | Msp_1110 | CobN | 1.7E−68 | MTH_714 | magnesium chelatase | 0.0E+00 |
| | | | | | subunit | |
| MSM1716 | Msp_0590 | member of asn/thr-rich large protein | 2.5E−16 | MTH_717 | unknown | 3.9E−25 |
| | | family | | | | |
| MSM1717 | Msp_1105 | predicted transporter | 1.9E−52 | MTH_672 | unknown | 2.3E−52 |
| MSM1718 | Msp_1106 | conserved hypothetical membrane- | 2.0E−50 | MTH_671 | unknown | 3.7E−61 |
| | | spanning protein | | | | |
| MSM1719 | Msp_1107 | conserved hypothetical membrane- | 4.1E−25 | MTH_670 | unknown | 1.2E−32 |
| | | spanning protein | | | | |
| MSM1720 | Msp_1533 | RpoM1 | 7.3E−28 | MTH_1314 | transcription elongation | 8.6E−30 |
| | | | | | factor TFIIS | |
| MSM1721 | NONE | | | NONE | | |
| MSM1722 | Msp_0965 | predicted nitroreductase | 6.9E−16 | MTH_120 | NADPH-oxidoreductase | 7.3E−33 |
| MSM1723 | Msp_1238 | N(5),N(10)- | 6.7E−105 | NONE | N5,N10-methenyl- | 2.1E−138 |
| | | methenyltetrahydromethanopterin | | | tetrahydromethanopterin | |
| | | cyclohydrolase | | | cyclohydrolase | |
| MSM1724 | Msp_0961 | hypothetical membrane-spanning | 3.1E−36 | MTH_1192 | conserved protein | 9.2E−25 |
| | | protein | | | | |
| MSM1725 | Msp_0961 | hypothetical membrane-spanning | 5.7E−28 | MTH_1192 | conserved protein | 1.6E−30 |
| | | protein | | | | |
| MSM1726 | Msp_0879 | hypothetical membrane-spanning | 9.0E−30 | MTH_1192 | conserved protein | 1.3E−25 |
| | | protein | | | | |
| MSM1727 | Msp_0844 | predicted multimeric flavodoxin | 1.2E−18 | MTH_135 | conserved protein | 1.9E−18 |
| MSM1728 | NONE | | | NONE | | |
| MSM1729 | Msp_0587 | hypothetical membrane-spanning | 5.0E−29 | MTH_520 | unknown | 3.9E−10 |
| | | protein | | | | |
| MSM1730 | Msp_0607 | hypothetical membrane-spanning | 6.5E−20 | MTH_1192 | conserved protein | 1.2E−26 |
| | | protein | | | | |
| MSM1731 | Msp_0714 | predicted short chain | 1.7E−115 | NONE | | |
| | | dehydrogenase | | | | |
| MSM1732 | Msp_1548 | hypothetical protein | 8.2E−07 | NONE | | |
| MSM1733 | Msp_0789 | rubrerythrin | 1.6E−39 | MTH_756 | rubrerythrin | 3.3E−43 |
| MSM1734 | Msp_1237 | ThyA | 8.9E−28 | MTH_774 | thymidylate synthase | 7.2E−26 |
| MSM1735 | Msp_0777 | member of asn/thr-rich large protein | 7.4E−116 | MTH_716 | cell surface glycoprotein | 1.4E−06 |
| | | family | | | (s-layer protein) | |
| MSM1736 | NONE | | | NONE | | |
| MSM1737 | NONE | | | NONE | | |
| MSM1738 | Msp_0154 | member of asn/thr-rich large protein | 2.3E−06 | NONE | | |
| | | family | | | | |
| MSM1739 | Msp_0987 | hypothetical membrane-spanning | 2.7E−07 | MTH_521 | unknown | 1.4E−05 |
| | | protein | | | | |
| MSM1740 | Msp_1323 | conserved hypothetical protein | 1.1E−16 | MTH_83 | O-linked GlcNAc | 4.7E−38 |
| | | | | | transferase | |
| MSM1741 | Msp_0113 | conserved hypothetical protein | 5.0E−05 | NONE | | |
| MSM1742 | Msp_0482 | hypothetical membrane-spanning | 2.7E−76 | NONE | | |
| | | protein | | | | |
| MSM1743 | Msp_0113 | conserved hypothetical protein | 4.1E−06 | NONE | | |
| MSM1744 | NONE | | | NONE | | |
| MSM1745 | Msp_0344 | predicted phosphate uptake | 2.0E−04 | NONE | | |
| | | regulator | | | | |
| MSM1746 | NONE | | | NONE | | |
| MSM1747 | Msp_0911 | member of asn/thr-rich large protein | 8.1E−06 | NONE | | |
| | | family | | | | |
| MSM1748 | NONE | | | NONE | | |
| MSM1749 | NONE | | | NONE | | |
| MSM1750 | NONE | | | NONE | | |
| MSM1751 | Msp_0113 | conserved hypothetical protein | 6.3E−15 | NONE | | |
| MSM1752 | Msp_0702 | conserved hypothetical protein | 1.2E−59 | MTH_1210 | mrr restriction system | 3.4E−42 |
| | | | | | related protein | |
| MSM1753 | Msp_0465 | conserved hypothetical membrane- | 6.7E−04 | NONE | | |
| | | spanning protein | | | | |
| MSM1754 | Msp_1328 | putative ATP-dependent protease | 3.6E−06 | NONE | | |
| | | La | | | | |
| MSM1755 | Msp_0219 | conserved hypothetical protein | 6.7E−04 | NONE | | |
| MSM1756 | Msp_0976 | hypothetical protein | 2.8E−05 | NONE | | |
| MSM1757 | NONE | | | NONE | | |
| MSM1758 | NONE | | | NONE | | |
| MSM1759 | NONE | | | NONE | | |
| MSM1760 | NONE | | | NONE | | |
| MSM1761 | Msp_0113 | conserved hypothetical protein | 7.6E−07 | MTH_540 | intracellular protein | 2.7E−05 |
| | | | | | transport protein | |
| MSM1762 | NONE | | | NONE | | |
| MSM1763 | Msp_1533 | RpoM1 | 4.6E−10 | MTH_1314 | transcription elongation | 3.1E−09 |
| | | | | | factor TFIIS | |
| MSM1764 | Msp_0226 | hypothetical protein | 8.9E−04 | NONE | | |
| MSM1765 | NONE | | | NONE | | |
| MSM1766 | Msp_1323 | conserved hypothetical protein | 4.8E−15 | MTH_83 | O-linked GlcNAc | 3.4E−35 |
| | | | | | transferase | |
| MSM1767 | Msp_1548 | hypothetical protein | 1.3E−04 | NONE | | |
| MSM1768 | NONE | | | NONE | | |
| MSM1769 | Msp_0724 | hypothetical membrane-spanning | 2.1E−08 | MTH_1277 | unknown | 8.9E−05 |
| | | protein | | | | |
| MSM1770 | Msp_0934 | conserved hypothetical membrane- | 1.4E−17 | MTH_518 | conserved protein | 3.4E−19 |
| | | spanning protein | | | | |
| MSM1771 | Msp_0128 | predicted helicase | 5.0E−19 | MTH_511 | DNA helicase II | 1.1E−26 |
| MSM1772 | Msp_0725 | hypothetical protein | 4.0E−11 | MTH_470 | conserved protein | 1.2E−04 |
| MSM1773 | Msp_1548 | hypothetical protein | 4.3E−07 | MTH_521 | unknown | 7.7E−05 |
| MSM1774 | NONE | | | NONE | | |
| MSM1775 | NONE | | | NONE | | |
| MSM1776 | NONE | | | NONE | | |
| MSM1777 | Msp_0799 | predicted transcriptional regulator | 3.3E−05 | MTH_671 | unknown | 2.6E−04 |
| MSM1778 | Msp_0726 | hypothetical protein | 2.7E−69 | NONE | | |
| MSM1779 | Msp_0725 | hypothetical protein | 2.6E−119 | NONE | | |
| MSM1780 | Msp_1055 | hypothetical membrane-spanning | 1.1E−10 | MTH_1277 | unknown | 2.7E−06 |
| | | protein | | | | |
| MSM1781 | Msp_0725 | hypothetical protein | 2.4E−13 | MTH_470 | conserved protein | 1.4E−05 |
| MSM1782 | NONE | | | NONE | | |
| MSM1783 | NONE | | | NONE | | |
| MSM1784 | NONE | | | NONE | | |
| MSM1785 | NONE | | | NONE | | |
| MSM1786 | Msp_1323 | conserved hypothetical protein | 4.1E−07 | MTH_83 | O-linked GlcNAc | 6.9E−12 |
| | | | | | transferase | |
| MSM1787 | Msp_1323 | conserved hypothetical protein | 5.6E−09 | MTH_72 | O-linked GlcNAc | 3.6E−16 |
| | | | | | transferase | |
| MSM1788 | Msp_1323 | conserved hypothetical protein | 7.3E−11 | MTH_83 | O-linked GlcNAc | 2.0E−20 |
| | | | | | transferase | |
| MSM1789 | Msp_0757 | predicted ATPase | 2.5E−08 | NONE | | |
| MSM1790 | Msp_0757 | predicted ATPase | 4.9E−08 | NONE | | |
| MSM1791 | NONE | | | MTH_512 | unknown | 1.1E−25 |
| MSM1792 | Msp_0764 | predicted nicotinate | 1.7E−193 | NONE | | |
| | | phosphoribosyltransferase | | | | |
| MSM1793 | NONE | | | NONE | | |
| MSM1794 | Msp_1103 | member of asn/thr-rich large protein | 1.5E−04 | MTH_512 | unknown | 1.2E−24 |
| | | family | | | | |
| MSM1795 | Msp_0757 | predicted ATPase | 1.7E−99 | NONE | | |

TABLE 9
Cluster of Orthologous Groups (COG) represented in the M. smithii proteome
A. Summary

| Number of M. smithii | | |
|---|---|---|
| genes in COG | Code | Functional Category |
| 136 | J | Translation |
| 60 | K | Transcription |
| 78 | L | Replication, Recombination and Repair |
| 3 | B | Chromatin Structure and Dynamics |
| 6 | D | Cell Cycle Control |
| 26 | V | Defense Mechanisms |
| 8 | T | Signal Transduction Mechanisms |
| 59 | M | Cell Wall/Membrane Biogenesis |
| 3 | N | Cell Motility |
| 1 | Z | Cytoskeleton |
| 17 | U | Intracellular Trafficking and Secretion |
| 41 | O | Post-translational Modification, Protein Turnover, |
| | | Chaperones |
| 121 | C | Energy Production and Conversion |
| 30 | G | Carbohydrate Transport and Metabolism |
| 82 | E | Amino Acid Transport and Metabolism |
| 42 | F | Nucleic Acid Transport and Metabolism |
| 92 | H | Coenzyme Transport and Metabolism |
| 18 | I | Lipid Transport and Metabolism |
| 57 | P | Inorganic Ion Transport and Metabolism |
| 1 | Q | Secondary Metabolites Biosynthesis, Transport and |
| | | Catabolism |
| 201 | R | General Function Prediction Only |
| 171 | S | Function Unknown |
| 491 | — | Not in COGs |

B. M. smithii genes in each COG

| | | |
|---|---|---|
| # in COG COG | Description | M. smithii gene(s) |

Translation (J)

| | | | |
|---|---|---|---|
| 1 | COG0008 | Glutamyl- and glutaminyl-tRNA synthetases | MSM1452 |
| 1 | COG0009 | Putative translation factor (SUA5) | MSM0612 |
| 1 | COG0012 | Predicted GTPase, probable translation factor | MSM1164 |
| 1 | COG0013 | Alanyl-tRNA synthetase | MSM0619 |
| 1 | COG0016 | Phenylalanyl-tRNA synthetase alpha subunit | MSM1478 |
| 1 | COG0017 | Aspartyl/asparaginyl-tRNA synthetases | MSM1236 |
| 1 | COG0018 | Arginyl-tRNA synthetase | MSM1231 |
| 1 | COG0023 | Translation initiation factor 1 (eIF-1/SUI1) and related proteins | MSM0754 |
| 1 | COG0024 | Methionine aminopeptidase | MSM1120 |
| 1 | COG0030 | Dimethyladenosine transferase (rRNA methylation) | MSM1374 |
| 1 | COG0042 | tRNA-dihydrouridine synthase | MSM0972 |
| 1 | COG0048 | Ribosomal protein S12 | MSM0901 |
| 1 | COG0049 | Ribosomal protein S7 | MSM0900 |
| 1 | COG0051 | Ribosomal protein S10 | MSM0897 |
| 1 | COG0060 | Isoleucyl-tRNA synthetase | MSM1341 |
| 1 | COG0064 | Asp-tRNAAsn/Glu-tRNAGln amidotransferase B subunit (PET112 | MSM1101 |
| | | homolog) | |
| 1 | COG0072 | Phenylalanyl-tRNA synthetase beta subunit | MSM0277 |
| 1 | COG0080 | Ribosomal protein L11 | MSM0623 |
| 1 | COG0081 | Ribosomal protein L1 | MSM0622 |
| 1 | COG0087 | Ribosomal protein L3 | MSM0762 |
| 1 | COG0088 | Ribosomal protein L4 | MSM0761 |
| 1 | COG0089 | Ribosomal protein L23 | MSM0760 |
| 1 | COG0090 | Ribosomal protein L2 | MSM0759 |
| 1 | COG0091 | Ribosomal protein L22 | MSM0757 |
| 1 | COG0092 | Ribosomal protein S3 | MSM0756 |
| 1 | COG0093 | Ribosomal protein L14 | MSM0751 |
| 1 | COG0094 | Ribosomal protein L5 | MSM0748 |
| 1 | COG0096 | Ribosomal protein S8 | MSM0746 |
| 1 | COG0097 | Ribosomal protein L6P/L9E | MSM0745 |
| 1 | COG0098 | Ribosomal protein S5 | MSM0741 |
| 1 | COG0099 | Ribosomal protein S13 | MSM1425 |
| 1 | COG0100 | Ribosomal protein S11 | MSM1427 |
| 1 | COG0101 | Pseudouridylate synthase | MSM0855 |
| 1 | COG0102 | Ribosomal protein L13 | MSM1430 |
| 1 | COG0103 | Ribosomal protein S9 | MSM1431 |
| 1 | COG0124 | Histidyl-tRNA synthetase | MSM1181 |
| 1 | COG0130 | Pseudouridine synthase | MSM0732 |
| 1 | COG0143 | Methionyl-tRNA synthetase | MSM0071 |
| 1 | COG0154 | Asp-tRNAAsn/Glu-tRNAGln amidotransferase A subunit and | MSM1253 |
| | | related amidases | |
| 1 | COG0162 | Tyrosyl-tRNA synthetase | MSM0513 |
| 1 | COG0172 | Seryl-tRNA synthetase | MSM1710 |
| 1 | COG0180 | Tryptophanyl-tRNA synthetase | MSM0216 |
| 1 | COG0182 | Predicted translation initiation factor 2B subunit, eIF-2B | MSM0804 |
| | | alpha/beta/delta family | |
| 1 | COG0184 | Ribosomal protein S15P/S13E | MSM1194 |
| 1 | COG0185 | Ribosomal protein S19 | MSM0758 |
| 1 | COG0186 | Ribosomal protein S17 | MSM0752 |
| 1 | COG0197 | Ribosomal protein L16/L10E | MSM0989 |
| 1 | COG0198 | Ribosomal protein L24 | MSM0750 |
| 1 | COG0199 | Ribosomal protein S14 | MSM0747 |
| 1 | COG0200 | Ribosomal protein L15 | MSM0739 |
| 1 | COG0215 | Cysteinyl-tRNA synthetase | MSM0268 |
| 1 | COG0231 | Translation elongation factor P (EF-P)/translation initiation factor | MSM0877 |
| | | 5A (eIF-5A) | |
| 1 | COG0244 | Ribosomal protein L10 | MSM0621 |
| 1 | COG0255 | Ribosomal protein L29 | MSM0755 |
| 1 | COG0256 | Ribosomal protein L18 | MSM0742 |
| 1 | COG0293 | 23S rRNA methylase | MSM0508 |
| 1 | COG0343 | Queuine/archaeosine tRNA-ribosyltransferase | MSM1557 |
| 1 | COG0423 | Glycyl-tRNA synthetase (class II) | MSM0403 |
| 1 | COG0441 | Threonyl-tRNA synthetase | MSM1214 |
| 1 | COG0442 | Prolyl-tRNA synthetase | MSM0287 |
| 1 | COG0480 | Translation elongation factors (GTPases) | MSM0899 |
| 1 | COG0495 | Leucyl-tRNA synthetase | MSM1172 |
| 1 | COG0522 | Ribosomal protein S4 and related proteins | MSM1426 |
| 1 | COG0525 | Valyl-tRNA synthetase | MSM0275 |
| 1 | COG0532 | Translation initiation factor 2 (IF-2; GTPase) | MSM0202 |
| 1 | COG0565 | rRNA methylase | MSM0394 |
| 1 | COG0621 | 2-methylthioadenine synthetase | MSM0845 |
| 1 | COG0689 | RNase PH | MSM0242 |
| 1 | COG1093 | Translation initiation factor 2, alpha subunit (eIF-2alpha) | MSM1133 |
| 1 | COG1096 | Predicted RNA-binding protein (consists of S1 domain and a Zn- | MSM1357 |
| | | ribbon domain) | |
| 1 | COG1097 | RNA-binding protein Rrp4 and related proteins (contain S1 domain | MSM0243 |
| | | and KH domain) | |
| 1 | COG1258 | Predicted pseudouridylate synthase | MSM1361 |
| 1 | COG1325 | Predicted exosome subunit | MSM0297 |
| 1 | COG1358 | Ribosomal protein HS6-type (S12/L30/L7a) | MSM0206 |
| 1 | COG1369 | RNase P/RNase MRP subunit POP5 | MSM0246 |
| 1 | COG1383 | Ribosomal protein S17E | MSM0833 |
| 1 | COG1384 | Lysyl-tRNA synthetase (class I) | MSM1387 |
| 1 | COG1471 | Ribosomal protein S4E | MSM0749 |
| 1 | COG1491 | Predicted RNA-binding protein | MSM1375 |
| 1 | COG1498 | Protein implicated in ribosomal biogenesis, Nop56p homolog | MSM1046 |
| 1 | COG1500 | Predicted exosome subunit | MSM0244 |
| 1 | COG1503 | Peptide chain release factor 1 (eRF1) | MSM0891 |
| 1 | COG1514 | 2′-5′ RNA ligase | MSM0054 |
| 1 | COG1534 | Predicted RNA-binding protein containing KH domain, possibly | MSM0710 |
| | | ribosomal protein | |
| 2 | COG1549 | Queuine tRNA-ribosyltransferases, contain PUA domain | MSM0633, MSM0797 |
| 1 | COG1552 | Ribosomal protein L40E | MSM0125 |
| 1 | COG1588 | RNase P/RNase MRP subunit p29 | MSM0753 |
| 1 | COG1601 | Translation initiation factor 2, beta subunit (eIF-2beta)/eIF-5 N- | MSM0511 |
| | | terminal domain | |
| 1 | COG1603 | RNase P/RNase MRP subunit p30 | MSM0247 |
| 1 | COG1631 | Ribosomal protein L44E | MSM1135 |
| 1 | COG1632 | Ribosomal protein L15E | MSM0298 |
| 1 | COG1670 | Acetyltransferases, including N-acetylases of ribosomal proteins | MSM1573 |
| 1 | COG1676 | tRNA splicing endonuclease | MSM0217 |
| 1 | COG1717 | Ribosomal protein L32E | MSM0744 |
| 1 | COG1727 | Ribosomal protein L18E | MSM1429 |
| 1 | COG1736 | Diphthamide synthase subunit DPH2 | MSM1358 |
| 1 | COG1746 | tRNA nucleotidyltransferase (CCA-adding enzyme) | MSM0053 |
| 1 | COG1798 | Diphthamide biosynthesis methyltransferase | MSM0801 |
| 1 | COG1841 | Ribosomal protein L30/L7E | MSM0740 |
| 1 | COG1867 | N2,N2-dimethylguanosine tRNA methyltransferase | MSM1031 |
| 1 | COG1889 | Fibrillarin-like rRNA methylase | MSM1047 |
| 1 | COG1890 | Ribosomal protein S3AE | MSM0661 |
| 1 | COG1911 | Ribosomal protein L30E | MSM0907 |
| 1 | COG1976 | Translation initiation factor 6 (eIF-6) | MSM0704 |
| 1 | COG1997 | Ribosomal protein L37AE/L43A | MSM1630 |
| 1 | COG1998 | Ribosomal protein S27AE | MSM0193 |
| 1 | COG2004 | Ribosomal protein S24E | MSM0194 |
| 1 | COG2007 | Ribosomal protein S8E | MSM1486 |
| 1 | COG2016 | Predicted RNA-binding protein (contains PUA domain) | MSM0183 |
| 1 | COG2023 | RNase P subunit RPR2 | MSM0711 |
| 1 | COG2051 | Ribosomal protein S27E | MSM1134 |
| 1 | COG2053 | Ribosomal protein S28E/S33 | MSM0205 |
| 1 | COG2075 | Ribosomal protein L24E | MSM0204 |
| 1 | COG2092 | Translation elongation factor EF-1beta | MSM0602 |
| 1 | COG2097 | Ribosomal protein L31E | MSM0705 |
| 1 | COG2117 | Predicted subunit of tRNA(5-methylaminomethyl-2-thiouridylate) | MSM0707 |
| | | methyltransferase, contains the PP-loop ATPase domain | |
| 1 | COG2123 | RNase PH-related exoribonuclease | MSM0241 |
| 1 | COG2125 | Ribosomal protein S6E (S10) | MSM0201 |
| 1 | COG2126 | Ribosomal protein L37E | MSM0181 |
| 1 | COG2139 | Ribosomal protein L21E | MSM1377 |
| 1 | COG2147 | Ribosomal protein L19E | MSM0743 |
| 1 | COG2157 | Ribosomal protein L20A (L18A) | MSM0703 |
| 1 | COG2163 | Ribosomal protein L14E/L6E/L27E | MSM0733 |
| 1 | COG2167 | Ribosomal protein L39E | MSM0706 |
| 1 | COG2174 | Ribosomal protein L34E | MSM0735 |
| 1 | COG2238 | Ribosomal protein S19E (S16A) | MSM0709 |
| 1 | COG2260 | Predicted Zn-ribbon RNA-binding protein | MSM1132 |
| 1 | COG2263 | Predicted RNA methylase | MSM0764 |
| 1 | COG2511 | Archaeal Glu-tRNA Gln amidotransferase subunit E (contains GAD | MSM0335 |
| | | domain) | |
| 1 | COG2519 | tRNA(1-methyladenosine) methyltransferase and related | MSM1173 |
| | | methyltransferases | |
| 1 | COG2888 | Predicted Zn-ribbon RNA-binding protein with a function in | MSM0603 |
| | | translation | |
| 1 | COG2890 | Methylase of polypeptide chain release factors | MSM1373 |
| 1 | COG3277 | RNA-binding protein involved in rRNA processing | MSM0425 |
| 1 | COG5256 | Translation elongation factor EF-1alpha (GTPase) | MSM0898 |
| 1 | COG5257 | Translation initiation factor 2, gamma subunit (eIF-2gamma; | MSM0200 |
| | | GTPase) | |

Transcription (K)

| | | | |
|---|---|---|---|
| 2 | COG0085 | DNA-directed RNA polymerase, beta subunit/140 kD subunit | MSM0910, MSM0911 |
| 2 | COG0086 | DNA-directed RNA polymerase, beta′ subunit/160 kD subunit | MSM0908, MSM0909 |
| 1 | COG0195 | Transcription elongation factor | MSM0906 |
| 1 | COG0202 | DNA-directed RNA polymerase, alpha subunit/40 kD subunit | MSM1428 |
| 1 | COG0250 | Transcription antiterminator | MSM0624 |
| 1 | COG0571 | dsRNA-specific ribonuclease | MSM0176 |
| 1 | COG0583 | Transcriptional regulator | MSM1390 |
| 3 | COG0640 | Predicted transcriptional regulators | MSM0819, MSM1126, |
| | | | MSM1350 |
| 1 | COG0789 | Predicted transcriptional regulators | MSM0949 |
| 1 | COG0846 | NAD-dependent protein deacetylases, SIR2 family | MSM1087 |
| 1 | COG0864 | Predicted transcriptional regulators containing the CopG/Arc/MetJ | MSM0364 |
| | | DNA-binding domain and a metal-binding domain | |
| 1 | COG1095 | DNA-directed RNA polymerase, subunit E′ | MSM0197 |
| 1 | COG1293 | Predicted RNA-binding protein homologous to eukaryotic snRNP | MSM0778 |
| 1 | COG1308 | Transcription factor homologous to NACalpha-BTF3 | MSM0384 |
| 2 | COG1309 | Transcriptional regulator | MSM0094, MSM0650 |
| 1 | COG1321 | Mn-dependent transcriptional regulator | MSM0218 |
| 1 | COG1378 | Predicted transcriptional regulators | MSM1445 |
| 1 | COG1395 | Predicted transcriptional regulator | MSM0453 |
| 3 | COG1396 | Predicted transcriptional regulators | MSM0026, MSM0329, |
| | | | MSM1528 |
| 1 | COG1405 | Transcription initiation factor TFIIIB, Brf1 subunit/Transcription | MSM0424 |
| | | initiation factor TFIIB | |
| 1 | COG1476 | Predicted transcriptional regulators | MSM1150 |
| 1 | COG1497 | Predicted transcriptional regulator | MSM1499 |
| 1 | COG1522 | Transcriptional regulators | MSM1032 |
| 1 | COG1581 | Archaeal DNA-binding protein | MSM1245 |
| 3 | COG1594 | DNA-directed RNA polymerase, subunit M/Transcription elongation | MSM1354, MSM1720, |
| | | factor TFIIS | MSM1763 |
| 1 | COG1644 | DNA-directed RNA polymerase, subunit N (RpoN/RPB10) | MSM1432 |
| 1 | COG1675 | Transcription initiation factor IIE, alpha subunit | MSM0631 |
| 1 | COG1695 | Predicted transcriptional regulators | MSM1250 |
| 1 | COG1733 | Predicted transcriptional regulators | MSM0864 |
| 1 | COG1758 | DNA-directed RNA polymerase, subunit K/omega | MSM1433 |
| 1 | COG1761 | DNA-directed RNA polymerase, subunit L | MSM1356 |
| 1 | COG1777 | Predicted transcriptional regulators | MSM1107 |
| 1 | COG1813 | Predicted transcription factor, homolog of eukaryotic MBF1 | MSM0355 |
| 3 | COG1846 | Transcriptional regulators | MSM0413, MSM0600, |
| | | | MSM1230 |
| 2 | COG1958 | Small nuclear ribonucleoprotein (snRNP) homolog | MSM0182, MSM1220 |
| 1 | COG1996 | DNA-directed RNA polymerase, subunit RPC10 (contains C4-type | MSM1631 |
| | | Zn-finger) | |
| 1 | COG2012 | DNA-directed RNA polymerase, subunit H, RpoH/RPB5 | MSM0912 |
| 1 | COG2093 | DNA-directed RNA polymerase, subunit E″ | MSM0196 |
| 1 | COG2101 | TATA-box binding protein (TBP), component of TFIID and TFIIIB | MSM0720 |
| 1 | COG2183 | Transcriptional accessory protein | MSM1292 |
| 1 | COG2207 | AraC-type DNA-binding domain-containing proteins | MSM0775 |
| 1 | COG2524 | Predicted transcriptional regulator, contains C-terminal CBS | MSM1614 |
| | | domains | |
| 2 | COG2865 | Predicted transcriptional regulator containing an HTH domain and | MSM0540, MSM1315 |
| | | an uncharacterized domain shared with the mammalian protein | |
| | | Schlafen | |
| 1 | COG4008 | Predicted metal-binding transcription factor | MSM0969 |
| 3 | COG4742 | Predicted transcriptional regulator | MSM0404, MSM0817, |
| | | | MSM0818 |

Replication, Recombination and Repair (L)

| | | | |
|---|---|---|---|
| 2 | COG0084 | Mg-dependent DNase | MSM0097, MSM0416 |
| 1 | COG0122 | 3-methyladenine DNA glycosylase/8-oxoguanine DNA glycosylase | MSM1365 |
| 1 | COG0164 | Ribonuclease HII | MSM0979 |
| 2 | COG0177 | Predicted EndoIII-related endonuclease | MSM0272, MSM1584 |
| 1 | COG0178 | Excinuclease ATPase subunit | MSM1581 |
| 2 | COG0188 | Type IIA topoisomerase (DNA gyrase/topo II, topoisomerase IV), A | MSM1353, MSM1775 |
| | | subunit | |
| 5 | COG0210 | Superfamily I DNA and RNA helicases | MSM0058, MSM0113, |
| | | | MSM0731, MSM1420, |
| | | | MSM1771 |
| 1 | COG0258 | 5′-3′ exonuclease (including N-terminal domain of Poll) | MSM0725 |
| 1 | COG0270 | Site-specific DNA methylase | MSM0531 |
| 1 | COG0322 | Nuclease subunit of the excinuclease complex | MSM1695 |
| 1 | COG0350 | Methylated DNA-protein cysteine methyltransferase | MSM1185 |
| 1 | COG0358 | DNA primase (bacterial type) | MSM0427 |
| 2 | COG0417 | DNA polymerase elongation subunit (family B) | MSM1041, MSM1481 |
| 3 | COG0419 | ATPase involved in DNA repair | MSM0120, MSM0693, |
| | | | MSM1761 |
| 1 | COG0420 | DNA repair exonuclease | MSM0121 |
| 2 | COG0468 | RecA/RadA recombinase | MSM0611, MSM1333 |
| 2 | COG0470 | ATPase involved in DNA replication | MSM1176, MSM1177 |
| 1 | COG0550 | Topoisomerase IA | MSM0717 |
| 1 | COG0556 | Helicase subunit of the DNA excision repair complex | MSM1579 |
| 3 | COG0582 | Integrase | MSM0428, MSM1640, |
| | | | MSM1742 |
| 1 | COG0592 | DNA polymerase sliding clamp subunit (PCNA homolog) | MSM1137 |
| 2 | COG0608 | Single-stranded DNA-specific exonuclease | MSM1193, MSM1500 |
| 1 | COG0648 | Endonuclease IV | MSM0963 |
| 1 | COG0708 | Exonuclease III | MSM1479 |
| 1 | COG1041 | Predicted DNA modification methylase | MSM0352 |
| 1 | COG1107 | Archaea-specific RecJ-like exonuclease, contains DnaJ-type Zn | MSM0260 |
| | | finger domain | |
| 1 | COG1111 | ERCC4-like helicases | MSM1187 |
| 2 | COG1112 | Superfamily I DNA and RNA helicases and helicase subunits | MSM1081, MSM1694 |
| 1 | COG1193 | Mismatch repair ATPase (MutS family) | MSM0524 |
| 1 | COG1241 | Predicted ATPase involved in replication control, Cdc46/Mcm | MSM0510 |
| | | family | |
| 1 | COG1311 | Archaeal DNA polymerase II, small subunit/DNA polymerase delta, | MSM1271 |
| | | subunit B | |
| 1 | COG1343 | Uncharacterized protein predicted to be involved in DNA repair | MSM0163 |
| 1 | COG1389 | DNA topoisomerase VI, subunit B | MSM0955 |
| 2 | COG1468 | RecB family exonuclease | MSM0165, MSM1059 |
| 2 | COG1518 | Uncharacterized protein predicted to be involved in DNA repair | MSM0023, MSM0164 |
| 1 | COG1525 | Micrococcal nuclease (thermonuclease) homologs | MSM1495 |
| 1 | COG1533 | DNA repair photolyase | MSM0543 |
| 1 | COG1570 | Exonuclease VII, large subunit | MSM0001 |
| 1 | COG1583 | Uncharacterized protein predicted to be involved in DNA repair | MSM0170 |
| | | (RAMP superfamily) | |
| 1 | COG1591 | Holliday junction resolvase - archaeal type | MSM1098 |
| 1 | COG1599 | Single-stranded DNA-binding replication protein A (RPA), large (70 kD) | MSM1332 |
| | | subunit and related ssDNA-binding proteins | |
| 1 | COG1637 | Predicted nuclease of the RecB family | MSM0497 |
| 1 | COG1688 | Uncharacterized protein predicted to be involved in DNA repair | MSM0167 |
| | | (RAMP superfamily) | |
| 1 | COG1697 | DNA topoisomerase VI, subunit A | MSM0956 |
| 1 | COG1793 | ATP-dependent DNA ligase | MSM0645 |
| 1 | COG1857 | Uncharacterized protein predicted to be involved in DNA repair | MSM0168 |
| 1 | COG1933 | Archaeal DNA polymerase II, large subunit | MSM1384 |
| 1 | COG2219 | Eukaryotic-type DNA primase, large subunit | MSM0073 |
| 1 | COG2231 | Uncharacterized protein related to Endonuclease III | MSM1475 |
| 2 | COG3335 | Transposase and inactivated derivatives | MSM0460, MSM1589 |
| 1 | COG3359 | Predicted exonuclease | MSM0138 |
| 2 | COG3415 | Transposase and inactivated derivatives | MSM0458, MSM1588 |
| 5 | COG3464 | Transposase and inactivated derivatives | MSM0087, MSM0230, |
| | | | MSM0396, MSM1093, |
| | | | MSM1566 |
| 1 | COG3666 | Transposase and inactivated derivatives | MSM1523 |

Chromatin Structure and Dynamics (B)

| | | | |
|---|---|---|---|
| 3 | COG2036 | Histones H3 and H4 | MSM0213, MSM0844, |
| | | | MSM1260 |

Cell Cycle Control (D)

| | | | |
|---|---|---|---|
| 3 | COG0037 | Predicted ATPase of the PP-loop superfamily implicated in cell | MSM0553, MSM1028, |
| | | cycle control | MSM1178 |
| 1 | COG0489 | ATPases involved in chromosome partitioning | MSM0045 |
| 1 | COG1077 | Actin-like ATPase involved in cell morphogenesis | MSM0980 |
| 1 | COG1192 | ATPases involved in chromosome partitioning | MSM1241 |

Defense Mechanisms (V)

| | | | |
|---|---|---|---|
| 5 | COG0534 | Na+-driven multidrug efflux pump | MSM0152, MSM0252, |
| | | | MSM0414, MSM1228, |
| | | | MSM1229 |
| 2 | COG0577 | ABC-type antimicrobial peptide transport system, permease | MSM0856, MSM1400 |
| | | component | |
| 2 | COG0732 | Restriction endonuclease S subunits | MSM0157, MSM0158 |
| 2 | COG0842 | ABC-type multidrug transport system, permease component | MSM1248, MSM1484 |
| 6 | COG1002 | Type II restriction enzyme, methylase subunits | MSM1743, MSM1744, |
| | | | MSM1745, MSM1746, |
| | | | MSM1747, MSM1748 |
| 3 | COG1131 | ABC-type multidrug transport system, ATPase component | MSM0593, MSM1249, |
| | | | MSM1483 |
| 2 | COG1132 | ABC-type multidrug transport system, ATPase and permease | MSM0773, MSM0774 |
| | | components | |
| 1 | COG1136 | ABC-type antimicrobial peptide transport system, ATPase | MSM0857 |
| | | component | |
| 1 | COG1715 | Restriction endonuclease | MSM1752 |
| 1 | COG1968 | Uncharacterized bacitracin resistance protein | MSM1201 |
| 1 | COG4845 | Chloramphenicol O-acetyltransferase | MSM0047 |

Signal Transduction Mechanisms (T)

| | | | |
|---|---|---|---|
| 3 | COG0589 | Universal stress protein UspA and related nucleotide-binding | MSM0485, MSM0887, |
| | | proteins | MSM1699 |
| 5 | COG3448 | CBS-domain-containing membrane protein | MSM0305, MSM0484, |
| | | | MSM0790, MSM1053, |
| | | | MSM1054 |

Cell Wall/Membrane Biogenesis (M)

| | | | |
|---|---|---|---|
| 1 | COG0381 | UDP-N-acetylglucosamine 2-epimerase | MSM0853 |
| 3 | COG0399 | Predicted pyridoxal phosphate-dependent enzyme apparently | MSM0347, MSM1030, |
| | | involved in regulation of cell wall biogenesis | MSM1536 |
| 4 | COG0438 | Glycosyltransferase | MSM0836, MSM1313, |
| | | | MSM1317, MSM1322 |
| 1 | COG0449 | Glucosamine 6-phosphate synthetase, contains amidotransferase | MSM1551 |
| | | and phosphosugar isomerase domains | |
| 14 | COG0463 | Glycosyltransferases involved in cell wall biogenesis | MSM0423, MSM1290, |
| | | | MSM1294, MSM1297, |
| | | | MSM1310, MSM1311, |
| | | | MSM1312, MSM1316, |
| | | | MSM1323, MSM1324, |
| | | | MSM1328, MSM1545, |
| | | | MSM1623, MSM1627 |
| 2 | COG0472 | UDP-N-acetylmuramyl pentapeptide phosphotransferase/UDP- | MSM0066, MSM0360 |
| | | Nacetylglucosamine-1-phosphate transferase | |
| 1 | COG0562 | UDP-galactopyranose mutase | MSM1502 |
| 1 | COG0668 | Small-conductance mechanosensitive channel | MSM0493 |
| 1 | COG0677 | UDP-N-acetyl-D-mannosaminuronate dehydrogenase | MSM1303 |
| 1 | COG0707 | pfam match to MurG; not predicted to be a carbohydrate active | MSM0638 |
| | | enzyme by CAZy | |
| 1 | COG0750 | Predicted membrane-associated Zn-dependent proteases 1 | MSM1344 |
| 3 | COG0769 | UDP-N-acetylmuramyl tripeptide synthase | MSM0359, MSM1139, |
| | | | MSM1570 |
| 1 | COG0770 | UDP-N-acetylmuramyl pentapeptide synthase | MSM0880 |
| 1 | COG0771 | UDP-N-acetylmuramoylalanine-D-glutamate ligase | MSM0118 |
| 1 | COG0773 | UDP-N-acetylmuramate-alanine ligase | MSM1190 |
| 1 | COG0794 | Predicted sugar phosphate isomerase involved in capsule | MSM1391 |
| | | formation | |
| 1 | COG1004 | Predicted UDP-glucose 6-dehydrogenase | MSM1612 |
| 1 | COG1083 | CMP-N-acetylneuraminic acid synthetase | MSM0944 |
| 1 | COG1087 | UDP-glucose 4-epimerase | MSM1702 |
| 1 | COG1088 | dTDP-D-glucose 4,6-dehydratase | MSM1309 |
| 1 | COG1091 | dTDP-4-dehydrorhamnose reductase | MSM1304 |
| 1 | COG1209 | dTDP-glucose pyrophosphorylase | MSM1307 |
| 1 | COG1210 | UDP-glucose pyrophosphorylase | MSM1604 |
| 1 | COG1861 | Spore coat polysaccharide biosynthesis protein F, CMP-KDO | MSM1537 |
| | | synthetase homolog | |
| 1 | COG1887 | Putative glycosyl/glycerophosphate transferases involved in | MSM1327 |
| | | teichoic acid biosynthesis TagF/TagB/EpsJ/RodC | |
| 1 | COG1898 | dTDP-4-dehydrorhamnose 3,5-epimerase and related enzymes | MSM1308 |
| 1 | COG2089 | Sialic acid synthase | MSM1539 |
| 1 | COG2148 | Sugar transferases involved in lipopolysaccharide synthesis | MSM1331 |
| 1 | COG2222 | Predicted phosphosugar isomerases | MSM0872 |
| 2 | COG2230 | Cyclopropane fatty acid synthase and related methyltransferases | MSM0274, MSM0490 |
| 1 | COG2843 | Putative enzyme of poly-gamma-glutamate biosynthesis (capsule | MSM0700 |
| | | formation) | |
| 1 | COG3049 | Penicillin V acylase and related amidases | MSM0986 |
| 3 | COG3475 | LPS biosynthesis protein | MSM1512, MSM1515, |
| | | | MSM1544 |
| 1 | COG3764 | Sortase (surface protein transpeptidase) | MSM0984 |
| 1 | COG3980 | Spore coat polysaccharide biosynthesis protein, predicted | MSM1538 |
| | | glycosyltransferase | |

Cell Motility (N)

| | | | |
|---|---|---|---|
| 1 | COG3351 | Putative archaeal flagellar protein D/E | MSM0137 |
| 2 | COG5651 | PPE-repeat proteins | MSM1586, MSM1590 |

Cytoskeleton (Z)

| | | | |
|---|---|---|---|
| 1 | COG5023 | Tubulin | MSM1794 |

Intracellular Trafficking and Secretion (U)

| | | | |
|---|---|---|---|
| 1 | COG0201 | Preprotein translocase subunit SecY | MSM0738 |
| 1 | COG0541 | Signal recognition particle GTPase | MSM1360 |
| 1 | COG0552 | Signal recognition particle GTPase | MSM0701 |
| 2 | COG0681 | Signal peptidase I | MSM0232, MSM1232 |
| 3 | COG0811 | Biopolymer transport proteins | MSM0978, MSM1401, |
| | | | MSM1718 |
| 1 | COG0848 | Biopolymer transport protein | MSM0977 |
| 1 | COG1400 | Signal recognition particle 19 kDa protein | MSM1501 |
| 1 | COG2443 | Preprotein translocase subunit Sss1 | MSM0625 |
| 2 | COG3210 | Large exoproteins involved in heme utilization or adhesion | MSM0461, MSM1398 |
| 1 | COG4023 | Preprotein translocase subunit Sec61beta | MSM1363 |
| 1 | COG4962 | Flp pilus assembly protein, ATPase CpaF | MSM0597 |
| 2 | COG4965 | Flp pilus assembly protein TadB | MSM0471, MSM0596 |

Post-translational Modification, Protein Turnover, Chaperones (O)

| | | | |
|---|---|---|---|
| 1 | COG0068 | Hydrogenase maturation factor | MSM1106 |
| 1 | COG0071 | Molecular chaperone (small heat shock protein) | MSM0870 |
| 1 | COG0225 | Peptide methionine sulfoxide reductase | MSM0582 |
| 1 | COG0298 | Hydrogenase maturation factor | MSM0636 |
| 1 | COG0309 | Hydrogenase maturation factor | MSM1492 |
| 1 | COG0396 | ABC-type transport system involved in Fe—S cluster assembly, | MSM1003 |
| | | ATPase component | |
| 1 | COG0409 | Hydrogenase maturation factor | MSM0945 |
| 1 | COG0443 | Molecular chaperone | MSM1109 |
| 3 | COG0459 | Chaperonin GroEL (HSP60 family) | MSM0220, MSM0826, |
| | | | MSM1533 |
| 1 | COG0464 | ATPases of the AAA+ class | MSM0642 |
| 1 | COG0484 | DnaJ-class molecular chaperone with C-terminal Zn finger domain | MSM1110 |
| 1 | COG0492 | Thioredoxin reductase | MSM0340 |
| 2 | COG0501 | Zn-dependent protease with chaperone function | MSM1174, MSM1203 |
| 1 | COG0533 | Metal-dependent proteases with possible chaperone activity | MSM1198 |
| 1 | COG0576 | Molecular chaperone GrpE (heat shock protein) | MSM1108 |
| 1 | COG0602 | Organic radical activating enzymes | MSM1055 |
| 2 | COG0638 | 20S proteasome, alpha and beta subunits | MSM0245, MSM1037 |
| 1 | COG0652 | Peptidyl-prolyl cis-trans isomerase (rotamase) - cyclophilin family | MSM1367 |
| 1 | COG0719 | ABC-type transport system involved in Fe—S cluster assembly, | MSM1002 |
| | | permease component | |
| 1 | COG0785 | Cytochrome c biogenesis protein | MSM0549 |
| 3 | COG0826 | Collagenase and related proteases | MSM0522, MSM0523, |
| | | | MSM1705 |
| 1 | COG1047 | FKBP-type peptidyl-prolyl cis-trans isomerases 2 | MSM0930 |
| 1 | COG1067 | Predicted ATP-dependent protease | MSM1569 |
| 3 | COG1180 | Pyruvate-formate lyase-activating enzyme | MSM0538, MSM0652, |
| | | | MSM1284 |
| 1 | COG1222 | ATP-dependent 26S proteasome regulatory subunit | MSM0354 |
| 1 | COG1382 | Prefoldin, chaperonin cofactor | MSM1634 |
| 1 | COG1397 | ADP-ribosylglycohydrolase | MSM1572 |
| 1 | COG1730 | Predicted prefoldin, molecular chaperone implicated in de novo | MSM0702 |
| | | protein folding | |
| 1 | COG1899 | Deoxyhypusine synthase | MSM1615 |
| 1 | COG1973 | Hydrogenase maturation factor | MSM1158 |
| 1 | COG2143 | Thioredoxin-related protein | MSM0550 |
| 1 | COG4070 | Predicted peptidyl-prolyl cis-trans isomerase (rotamase), | MSM0813 |
| | | cyclophilin family | |
| 1 | COG4930 | Predicted ATP-dependent Lon-type protease | MSM1754 |

Energy Production and Conversion (C)

| | | | |
|---|---|---|---|
| 1 | COG0045 | Succinyl-CoA synthetase, beta subunit | MSM0924 |
| 1 | COG0074 | Succinyl-CoA synthetase, alpha subunit | MSM0228 |
| 1 | COG0221 | Inorganic pyrophosphatase | MSM0198 |
| 1 | COG0240 | Glycerol-3-phosphate dehydrogenase | MSM1540 |
| 2 | COG0243 | Anaerobic dehydrogenases, typically selenocysteine-containing | MSM1404, MSM1463 |
| 1 | COG0247 | Fe—S oxidoreductase | MSM1625 |
| 1 | COG0371 | Glycerol dehydrogenase and related enzymes | MSM0286 |
| 1 | COG0372 | Citrate synthase | MSM0446 |
| 2 | COG0426 | Uncharacterized flavoproteins | MSM0222, MSM1349 |
| 1 | COG0479 | Succinate dehydrogenase/fumarate reductase, Fe—S protein | MSM0393 |
| | | subunit | |
| 1 | COG0636 | F0F1-type ATP synthase, subunit c/Archaeal/vacuolar-type H+- | MSM0439 |
| | | ATPase, subunit K | |
| 1 | COG0644 | Dehydrogenases (flavoproteins) | MSM1701 |
| 2 | COG0650 | Formate hydrogenlyase subunit 4 | MSM0317, MSM1062 |
| 3 | COG0674 | Pyruvate:ferredoxin oxidoreductase and related 2- | MSM0332, MSM0559, |
| | | oxoacid:ferredoxin oxidoreductases, alpha subunit | MSM0927 |
| 1 | COG0680 | Ni,Fe-hydrogenase maturation factor | MSM1123 |
| 3 | COG0716 | Flavodoxins | MSM0062, MSM0503, |
| | | | MSM0861 |
| 1 | COG0731 | Fe—S oxidoreductases | MSM0922 |
| 4 | COG0778 | Nitroreductase | MSM0445, MSM1293, |
| | | | MSM1574, MSM1722 |
| 1 | COG0822 | NifU homolog involved in Fe—S cluster formation | MSM0263 |
| 1 | COG1012 | NAD-dependent aldehyde dehydrogenases | MSM0467 |
| 3 | COG1013 | Pyruvate:ferredoxin oxidoreductase and related 2- | MSM0333, MSM0560, |
| | | oxoacid:ferredoxin oxidoreductases, beta subunit | MSM0926 |
| 3 | COG1014 | Pyruvate:ferredoxin oxidoreductase and related 2- | MSM0391, MSM0557, |
| | | oxoacid:ferredoxin oxidoreductases, gamma subunit | MSM0925 |
| 1 | COG1029 | Formylmethanofuran dehydrogenase subunit B | MSM1412 |
| 2 | COG1032 | Fe—S oxidoreductase | MSM0696, MSM0787 |
| 4 | COG1035 | Coenzyme F420-reducing hydrogenase, beta subunit | MSM0135, MSM1121, |
| | | | MSM1405, MSM1462 |
| 1 | COG1036 | Archaeal flavoproteins | MSM1338 |
| 1 | COG1042 | Acyl-CoA synthetase (NDP forming) | MSM1471 |
| 1 | COG1053 | Succinate dehydrogenase/fumarate reductase, flavoprotein subunit | MSM1258 |
| 1 | COG1139 | Uncharacterized conserved protein containing a ferredoxin-like | MSM1626 |
| | | domain | |
| 2 | COG1142 | Fe—S-cluster-containing hydrogenase components 2 | MSM0561, MSM0562 |
| 2 | COG1143 | Formate hydrogenlyase subunit 6/NADH:ubiquinone | MSM0998, MSM1065 |
| | | oxidoreductase 23 kD subunit (chain I) | |
| 1 | COG1144 | Pyruvate:ferredoxin oxidoreductase and related 2- | MSM0558 |
| | | oxoacid:ferredoxin oxidoreductases, delta subunit | |
| 12 | COG1145 | Ferredoxin | MSM0136, MSM0306, |
| | | | MSM0310, MSM0311, |
| | | | MSM0395, MSM0579, |
| | | | MSM0783, MSM0784, |
| | | | MSM1066, MSM1409, |
| | | | MSM1410, MSM1700 |
| 5 | COG1146 | Ferredoxin | MSM0085, MSM0209, |
| | | | MSM0331, MSM0928, |
| | | | MSM1408 |
| 2 | COG1148 | Heterodisulfide reductase, subunit A and related polyferredoxins | MSM0082, MSM1336 |
| 2 | COG1150 | Heterodisulfide reductase, subunit C | MSM0084, MSM0796 |
| 1 | COG1151 | 6Fe—6S prismane cluster-containing protein | MSM1446 |
| 1 | COG1153 | Formylmethanofuran dehydrogenase subunit D | MSM1411 |
| 1 | COG1155 | Archaeal/vacuolar-type H+-ATPase subunit A | MSM0435 |
| 1 | COG1156 | Archaeal/vacuolar-type H+-ATPase subunit B | MSM0434 |
| 1 | COG1229 | Formylmethanofuran dehydrogenase subunit A | MSM1413 |
| 1 | COG1249 | Pyruvate/2-oxoglutarate dehydrogenase complex, | MSM0637 |
| | | dihydrolipoamide dehydrogenase (E3) component, and related | |
| | | enzymes | |
| 1 | COG1269 | Archaeal/vacuolar-type H+-ATPase subunit I | MSM0440 |
| 1 | COG1304 | L-lactate dehydrogenase (FMN-dependent) and related alpha- | MSM1441 |
| | | hydroxy acid dehydrogenases | |
| 1 | COG1390 | Archaeal/vacuolar-type H+-ATPase subunit E | MSM0438 |
| 1 | COG1394 | Archaeal/vacuolar-type H+-ATPase subunit D | MSM0433 |
| 2 | COG1413 | FOG: HEAT repeat | MSM0372, MSM0501 |
| 1 | COG1436 | Archaeal/vacuolar-type H+-ATPase subunit F | MSM0436 |
| 2 | COG1526 | Uncharacterized protein required for formate dehydrogenase | MSM0295, MSM1392 |
| | | activity | |
| 1 | COG1527 | Archaeal/vacuolar-type H+-ATPase subunit C | MSM0437 |
| 2 | COG1592 | Rubrerythrin | MSM1348, MSM1733 |
| 1 | COG1600 | Uncharacterized Fe—S protein | MSM0609 |
| 1 | COG1625 | Fe—S oxidoreductase, related to NifB/MoaA family | MSM1020 |
| 2 | COG1773 | Rubredoxin | MSM0187, MSM0188 |
| 2 | COG1838 | Tartrate dehydratase beta subunit/Fumarate hydratase class I, C- | MSM0769, MSM0929 |
| | | terminal domain | |
| 2 | COG1908 | Coenzyme F420-reducing hydrogenase, delta subunit | MSM1001, MSM1461 |
| 2 | COG1941 | Coenzyme F420-reducing hydrogenase, gamma subunit | MSM1000, MSM1122 |
| 2 | COG1951 | Tartrate dehydratase alpha subunit/Fumarate hydratase class I, N- | MSM0447, MSM0563 |
| | | terminal domain | |
| 1 | COG2033 | Desulfoferrodoxin | MSM0262 |
| 2 | COG2037 | Formylmethanofuran:tetrahydromethanopterin formyltransferase | MSM0308, MSM1092 |
| 2 | COG2048 | Heterodisulfide reductase, subunit B | MSM0083, MSM0795 |
| 1 | COG2055 | Malate/L-lactate dehydrogenases | MSM1040 |
| 1 | COG2141 | Coenzyme F420-dependent N5,N10-methylene | MSM0542 |
| | | tetrahydromethanopterin reductase and related flavin-dependent | |
| | | oxidoreductases | |
| 1 | COG2191 | Formylmethanofuran dehydrogenase subunit E | MSM1396 |
| 1 | COG2218 | Formylmethanofuran dehydrogenase subunit C | MSM1414 |
| 1 | COG2710 | Nitrogenase molybdenum-iron protein, alpha and beta chains | MSM1160 |
| 1 | COG2811 | Archaeal/vacuolar-type H+-ATPase subunit H | MSM0441 |
| 2 | COG3259 | Coenzyme F420-reducing hydrogenase, alpha subunit | MSM0999, MSM1124 |
| 1 | COG3260 | Ni,Fe-hydrogenase III small subunit | MSM1064 |
| 1 | COG3261 | Ni,Fe-hydrogenase III large subunit | MSM1063 |
| 2 | COG4231 | Indolepyruvate ferredoxin oxidoreductase, alpha and beta subunits | MSM0392, MSM1460 |
| 1 | COG5016 | Pyruvate/oxaloacetate carboxyltransferase | MSM0939 |

Carbohydrate Transport and Metabolism (G)

| | | | |
|---|---|---|---|
| 1 | COG0057 | Glyceraldehyde-3-phosphate dehydrogenase/erythrose-4- | MSM0962 |
| | | phosphate dehydrogenase | |
| 1 | COG0063 | Predicted sugar kinase | MSM1091 |
| 1 | COG0120 | Ribose 5-phosphate isomerase | MSM0284 |
| 1 | COG0126 | 3-phosphoglycerate kinase | MSM0918 |
| 1 | COG0148 | Enolase | MSM1435 |
| 1 | COG0149 | Triosephosphate isomerase | MSM0919 |
| 1 | COG0235 | Ribulose-5-phosphate 4-epimerase and related epimerases and | MSM1270 |
| | | aldolases | |
| 1 | COG0483 | Archaeal fructose-1,6-bisphosphatase and related enzymes of | MSM0879 |
| | | inositol monophosphatase family | |
| 3 | COG0524 | Sugar kinases, ribokinase family | MSM0307, MSM1389, |
| | | | MSM1693 |
| 2 | COG0574 | Phosphoenolpyruvate synthase/pyruvate phosphate dikinase | MSM0823, MSM0988 |
| 1 | COG0580 | Glycerol uptake facilitator and related permeases (Major Intrinsic | MSM1085 |
| | | Protein Family) | |
| 2 | COG1082 | Sugar phosphate isomerases/epimerases | MSM1184, MSM1251 |
| 2 | COG1109 | Phosphomannomutase | MSM0648, MSM0656 |
| 1 | COG1363 | Cellulase M and related proteins | MSM0134 |
| 1 | COG1830 | DhnA-type fructose-1,6-bisphosphate aldolase and related enzymes | MSM0056 |
| 1 | COG1980 | Archaeal fructose 1,6-bisphosphatase | MSM0615 |
| 2 | COG2074 | 2-phosphoglycerate kinase | MSM0408, MSM0791 |
| 2 | COG2730 | Endoglucanase | MSM1051, MSM1125 |
| 2 | COG2814 | Arabinose efflux permease | MSM1459, MSM1465 |
| 2 | COG3635 | Predicted phosphoglycerate mutase, AP superfamily | MSM0153, MSM0657 |
| 1 | COG5297 | Cellobiohydrolase A (1,4-beta-cellobiosidase A) | MSM0958 |

Amino Acid Transport and Metabolism (E)

| | | | |
|---|---|---|---|
| 1 | COG0002 | Acetylglutamate semialdehyde dehydrogenase | MSM0860 |
| 1 | COG0006 | Xaa-Pro aminopeptidase | MSM0472 |
| 1 | COG0019 | Diaminopimelate decarboxylase | MSM1371 |
| 1 | COG0031 | Cysteine synthase | MSM0271 |
| 1 | COG0040 | ATP phosphoribosyltransferase | MSM1261 |
| 2 | COG0065 | 3-isopropylmalate dehydratase large subunit | MSM0723, MSM1300 |
| 2 | COG0066 | 3-isopropylmalate dehydratase small subunit | MSM0847, MSM1299 |
| 1 | COG0067 | Glutamate synthase domain 1 | MSM0370 |
| 2 | COG0069 | Glutamate synthase domain 2 | MSM0027, MSM0368 |
| 1 | COG0070 | Glutamate synthase domain 3 | MSM0369 |
| 2 | COG0075 | Serine-pyruvate aminotransferase/archaeal aspartate | MSM0677, MSM1513 |
| | | aminotransferase | |
| 1 | COG0076 | Glutamate decarboxylase and related PLP-dependent proteins | MSM0987 |
| 1 | COG0077 | Prephenate dehydratase | MSM1052 |
| 1 | COG0078 | Ornithine carbamoyltransferase | MSM1226 |
| 2 | COG0079 | Histidinol-phosphate/aromatic aminotransferase and cobyric acid | MSM0653, MSM1516 |
| | | decarboxylase | |
| 1 | COG0082 | Chorismate synthase | MSM1474 |
| 1 | COG0106 | Phosphoribosylformimino-5-aminoimidazole carboxamide | MSM0858 |
| | | ribonucleotide (ProFAR) isomerase | |
| 1 | COG0107 | Imidazoleglycerol-phosphate synthase | MSM1364 |
| 1 | COG0112 | Glycine/serine hydroxymethyltransferase | MSM1337 |
| 1 | COG0118 | Glutamine amidotransferase | MSM1159 |
| 3 | COG0119 | Isopropylmalate/homocitrate/citramalate synthases | MSM0350, MSM0722, |
| | | | MSM1246 |
| 1 | COG0128 | 5-enolpyruvylshikimate-3-phosphate synthase | MSM0273 |
| 1 | COG0131 | Imidazoleglycerol-phosphate dehydratase | MSM1206 |
| 1 | COG0133 | Tryptophan synthase beta chain | MSM1142 |
| 1 | COG0134 | Indole-3-glycerol phosphate synthase | MSM1143 |
| 1 | COG0136 | Aspartate-semialdehyde dehydrogenase | MSM0829 |
| 1 | COG0137 | Argininosuccinate synthase | MSM1084 |
| 1 | COG0139 | Phosphoribosyl-AMP cyclohydrolase | MSM1182 |
| 1 | COG0140 | Phosphoribosyl-ATP pyrophosphohydrolase | MSM1103 |
| 1 | COG0141 | Histidinol dehydrogenase | MSM1238 |
| 1 | COG0165 | Argininosuccinate lyase | MSM0192 |
| 1 | COG0169 | Shikimate 5-dehydrogenase | MSM1179 |
| 1 | COG0174 | Glutamine synthetase | MSM1418 |
| 1 | COG0253 | Diaminopimelate epimerase | MSM1372 |
| 1 | COG0287 | Prephenate dehydrogenase | MSM0641 |
| 1 | COG0289 | Dihydrodipicolinate reductase | MSM0830 |
| 1 | COG0334 | Glutamate dehydrogenase/leucine dehydrogenase | MSM0888 |
| 1 | COG0345 | Pyrroline-5-carboxylate reductase | MSM0089 |
| 1 | COG0346 | Lactoylglutathione lyase and related lyases | MSM1366 |
| 1 | COG0347 | Nitrogen regulatory protein PII | MSM0233 |
| 1 | COG0367 | Asparagine synthase (glutamine-hydrolyzing) | MSM0160 |
| 3 | COG0436 | Aspartate/tyrosine/aromatic aminotransferase | MSM0610, MSM0788, |
| | | | MSM1455 |
| 1 | COG0440 | Acetolactate synthase, small (regulatory) subunit | MSM1224 |
| 1 | COG0460 | Homoserine dehydrogenase | MSM0154 |
| 1 | COG0498 | Threonine synthase | MSM0214 |
| 1 | COG0527 | Aspartokinases | MSM0832 |
| 1 | COG0547 | Anthranilate phosphoribosyltransferase | MSM1144 |
| 1 | COG0548 | Acetylglutamate kinase | MSM0375 |
| 1 | COG0560 | Phosphoserine phosphatase | MSM0719 |
| 1 | COG0620 | Methionine synthase II (cobalamin-independent) | MSM0102 |
| 1 | COG0710 | 3-dehydroquinate dehydratase | MSM0231 |
| 1 | COG0747 | ABC-type dipeptide transport system, periplasmic component | MSM0300 |
| 1 | COG0765 | ABC-type amino acid transport system, permease component | MSM0806 |
| 1 | COG1045 | Serine acetyltransferase | MSM0270 |
| 1 | COG1104 | Cysteine sulfinate desulfinase/cysteine desulfurase and related | MSM0264 |
| | | enzymes | |
| 1 | COG1125 | ABC-type proline/glycine betaine transport systems, ATPase | MSM0990 |
| | | components | |
| 1 | COG1126 | ABC-type polar amino acid transport system, ATPase component | MSM0805 |
| 1 | COG1168 | Bifunctional PLP-dependent enzyme with beta-cystathionase and | MSM0044 |
| | | maltose regulon repressor activities | |
| 1 | COG1174 | ABC-type proline/glycine betaine transport systems, permease | MSM0991 |
| | | component | |
| 2 | COG1305 | Transglutaminase-like enzymes, putative cysteine proteases | MSM0219, MSM0786 |
| 1 | COG1465 | Predicted alternative 3-dehydroquinate synthase | MSM0055 |
| 1 | COG1605 | Chorismate mutase | MSM0834 |
| 1 | COG1812 | Archaeal S-adenosylmethionine synthetase | MSM1340 |
| 1 | COG1921 | Selenocysteine synthase [seryl-tRNASer selenium transferase] | MSM0767 |
| 1 | COG2021 | Homoserine acetyltransferase | MSM0496 |
| 1 | COG2061 | ACT-domain-containing protein, predicted allosteric regulator of | MSM0155 |
| | | homoserine dehydrogenase | |
| 1 | COG2303 | Choline dehydrogenase and related flavoproteins | MSM0865 |
| 1 | COG2423 | Predicted ornithine cyclodeaminase, mu-crystallin homolog | MSM1517 |
| 1 | COG2856 | Predicted Zn peptidase | MSM1529 |
| 2 | COG2873 | O-acetylhomoserine sulfhydrylase | MSM0174, MSM0265 |
| 1 | COG4992 | Ornithine/acetylornithine aminotransferase | MSM1368 |

Nucleic Acid Transport and Metabolism (F)

| | | | |
|---|---|---|---|
| 1 | COG0005 | Purine nucleoside phosphorylase | MSM0665 |
| 1 | COG0015 | Adenylosuccinate lyase | MSM1151 |
| 1 | COG0034 | Glutamine phosphoribosylpyrophosphate amidotransferase | MSM1704 |
| 1 | COG0035 | Uracil phosphoribosyltransferase | MSM0398 |
| 1 | COG0041 | Phosphoribosylcarboxyaminoimidazole (NCAIR) mutase | MSM1287 |
| 1 | COG0044 | Dihydroorotase and related cyclic amidohydrolases | MSM0997 |
| 1 | COG0046 | Phosphoribosylformylglycinamidine (FGAM) synthase, synthetase | MSM1342 |
| | | domain | |
| 1 | COG0047 | Phosphoribosylformylglycinamidine (FGAM) synthase, glutamine | MSM1549 |
| | | amidotransferase domain | |
| 1 | COG0104 | Adenylosuccinate synthase | MSM1468 |
| 1 | COG0105 | Nucleoside diphosphate kinase | MSM0203 |
| 2 | COG0125 | Thymidylate kinase | MSM0077, MSM0520 |
| 1 | COG0127 | Xanthosine triphosphate pyrophosphatase | MSM1195 |
| 1 | COG0150 | Phosphoribosylaminoimidazole (AIR) synthetase | MSM1039 |
| 1 | COG0151 | Phosphoribosylamine-glycine ligase | MSM1227 |
| 1 | COG0152 | Phosphoribosylaminoimidazolesuccinocarboxamide (SAICAR) | MSM1547 |
| | | synthase | |
| 1 | COG0167 | Dihydroorotate dehydrogenase | MSM1044 |
| 1 | COG0207 | Thymidylate synthase | MSM1734 |
| 1 | COG0274 | Deoxyribose-phosphate aldolase | MSM0843 |
| 1 | COG0284 | Orotidine-5′-phosphate decarboxylase | MSM1617 |
| 1 | COG0461 | Orotate phosphoribosyltransferase | MSM0821 |
| 1 | COG0503 | Adenine/guanine phosphoribosyltransferases and related PRPP- | MSM1359 |
| | | binding proteins | |
| 1 | COG0504 | CTP synthase (UTP-ammonia lyase) | MSM0147 |
| 1 | COG0516 | IMP dehydrogenase/GMP reductase | MSM1629 |
| 1 | COG0518 | GMP synthase - Glutamine amidotransferase domain | MSM0343 |
| 1 | COG0519 | GMP synthase, PP-ATPase domain/subunit | MSM0345 |
| 1 | COG0528 | Uridylate kinase | MSM0415 |
| 1 | COG0540 | Aspartate carbamoyltransferase, catalytic chain | MSM1263 |
| 2 | COG0717 | Deoxycytidine deaminase | MSM0402, MSM0687 |
| 1 | COG0856 | Orotate phosphoribosyltransferase homologs | MSM0883 |
| 1 | COG1001 | Adenine deaminase | MSM0874 |
| 1 | COG1051 | ADP-ribose pyrophosphatase | MSM1355 |
| 1 | COG1102 | Cytidylate kinase | MSM0734 |
| 1 | COG1328 | Oxygen-sensitive ribonucleoside-triphosphate reductase | MSM1383 |
| 1 | COG1437 | Adenylate cyclase, class 2 (thermophilic) | MSM0721 |
| 1 | COG1781 | Aspartate carbamoyltransferase, regulatory subunit | MSM0862 |
| 1 | COG1828 | Phosphoribosylformylglycinamidine (FGAM) synthase, PurS | MSM1548 |
| | | component | |
| 1 | COG1936 | Predicted nucleotide kinase (related to CMP and AMP kinases) | MSM0713 |
| 1 | COG2019 | Archaeal adenylate kinase | MSM0737 |
| 1 | COG2233 | Xanthine/uracil permeases | MSM0397 |
| 1 | COG3363 | Archaeal IMP cyclohydrolase | MSM0976 |

Coenzyme Transport and Metabolism (H)

| | | | |
|---|---|---|---|
| 1 | COG0001 | Glutamate-1-semialdehyde aminotransferase | MSM1233 |
| 1 | COG0007 | Uroporphyrinogen-III methylase | MSM1550 |
| 1 | COG0043 | 3-polyprenyl-4-hydroxybenzoate decarboxylase and related | MSM1286 |
| | | decarboxylases | |
| 1 | COG0054 | Riboflavin synthase beta-chain | MSM1296 |
| 1 | COG0108 | 3,4-dihydroxy-2-butanone 4-phosphate synthase | MSM1256 |
| 1 | COG0113 | Delta-aminolevulinic acid dehydratase | MSM1476 |
| 1 | COG0142 | Geranylgeranyl pyrophosphate synthase | MSM1443 |
| 1 | COG0157 | Nicotinate-nucleotide pyrophosphorylase | MSM0491 |
| 1 | COG0163 | 3-polyprenyl-4-hydroxybenzoate decarboxylase | MSM0237 |
| 1 | COG0171 | NAD synthase | MSM1171 |
| 1 | COG0181 | Porphobilinogen deaminase | MSM0881 |
| 1 | COG0237 | Dephospho-CoA kinase | MSM0141 |
| 1 | COG0294 | Dihydropteroate synthase and related enzymes | MSM0556 |
| 1 | COG0301 | Thiamine biosynthesis ATP pyrophosphatase | MSM0617 |
| 2 | COG0303 | Molybdopterin biosynthesis enzyme | MSM0950, MSM1343 |
| 1 | COG0311 | Predicted glutamine amidotransferase involved in pyridoxine | MSM0371 |
| | | biosynthesis | |
| 1 | COG0314 | Molybdopterin converting factor, large subunit | MSM0130 |
| 1 | COG0315 | Molybdenum cofactor biosynthesis enzyme | MSM1362 |
| 1 | COG0340 | Biotin-(acetyl-CoA carboxylase) ligase | MSM0766 |
| 1 | COG0351 | Hydroxymethylpyrimidine/phosphomethylpyrimidine kinase | MSM0289 |
| 1 | COG0352 | Thiamine monophosphate synthase | MSM0917 |
| 1 | COG0373 | Glutamyl-tRNA reductase | MSM0967 |
| 1 | COG0379 | Quinolinate synthase | MSM0494 |
| 1 | COG0382 | 4-hydroxybenzoate polyprenyltransferase and related | MSM0941 |
| | | prenyltransferases | |
| 1 | COG0407 | Uroporphyrinogen-III decarboxylase | MSM0518 |
| 2 | COG0422 | Thiamine biosynthesis protein ThiC | MSM0644, MSM1388 |
| 2 | COG0452 | Phosphopantothenoylcysteine synthetase/decarboxylase | MSM1048, MSM1049 |
| 1 | COG0476 | Dinucleotide-utilizing enzymes involved in molybdopterin and | MSM0729 |
| | | thiamine biosynthesis family 2 | |
| 1 | COG0499 | S-adenosylhomocysteine hydrolase | MSM0727 |
| 2 | COG0502 | Biotin synthase and related enzymes | MSM0573, MSM1099 |
| 1 | COG0521 | Molybdopterin biosynthesis enzymes | MSM0820 |
| 1 | COG0611 | Thiamine monophosphate kinase | MSM1283 |
| 1 | COG0684 | Demethylmenaquinone methyltransferase | MSM0426 |
| 1 | COG0720 | 6-pyruvoyl-tetrahydropterin synthase | MSM1056 |
| 1 | COG0746 | Molybdopterin-guanine dinucleotide biosynthesis protein A | MSM0240 |
| 1 | COG1010 | Precorrin-3B methylase | MSM1273 |
| 1 | COG1056 | Nicotinamide mononucleotide adenylyltransferase | MSM0129 |
| 1 | COG1270 | Cobalamin biosynthesis protein CobD/CbiB | MSM1266 |
| 2 | COG1429 | Cobalamin biosynthesis protein CobN and related Mg-chelatases | MSM1117, MSM1715 |
| 1 | COG1488 | Nicotinic acid phosphoribosyltransferase | MSM1792 |
| 2 | COG1492 | Cobyric acid synthase | MSM1254, MSM1565 |
| 2 | COG1541 | Coenzyme F390 synthetase | MSM0387, MSM1714 |
| 1 | COG1587 | Uroporphyrinogen-III synthase | MSM1504 |
| 1 | COG1648 | Siroheme synthase (precorrin-2 oxidase/ferrochelatase domain) | MSM0968 |
| 1 | COG1731 | Archaeal riboflavin synthase | MSM1622 |
| 1 | COG1763 | Molybdopterin-guanine dinucleotide biosynthesis protein | MSM1407 |
| 1 | COG1767 | Triphosphoribosyl-dephospho-CoA synthetase | MSM1477 |
| 1 | COG1797 | Cobyrinic acid a,c-diamide synthase | MSM1215 |
| 1 | COG1893 | Ketopantoate reductase | MSM0033 |
| 2 | COG1962 | Tetrahydromethanopterin S-methyltransferase, subunit H | MSM0627, MSM1007 |
| 1 | COG1985 | Pyrimidine reductase, riboflavin biosynthesis | MSM0065 |
| 1 | COG2038 | NaMN:DMB phosphoribosyltransferase | MSM1200 |
| 1 | COG2073 | Cobalamin biosynthesis protein CbiG | MSM1267 |
| 1 | COG2082 | Precorrin isomerase | MSM1234 |
| 1 | COG2099 | Precorrin-6x reductase | MSM0896 |
| 1 | COG2104 | Sulfur transfer protein involved in thiamine biosynthesis | MSM0552 |
| 1 | COG2145 | Hydroxyethylthiazole kinase, sugar kinase family | MSM0916 |
| 3 | COG2226 | Methylase involved in ubiquinone/menaquinone biosynthesis | MSM1448, MSM1558, MSM1564 |
| 1 | COG2241 | Precorrin-6B methylase 1 | MSM1167 |
| 1 | COG2242 | Precorrin-6B methylase 2 | MSM0238 |
| 1 | COG2243 | Precorrin-2 methylase | MSM1351 |
| 1 | COG2266 | GTP:adenosylcobinamide-phosphate guanylyltransferase | MSM1005 |
| 1 | COG2875 | Precorrin-4 methylase | MSM0101 |
| 1 | COG2896 | Molybdenum cofactor biosynthesis enzyme | MSM1406 |
| 1 | COG3161 | 4-hydroxybenzoate synthetase (chorismate lyase) | MSM0724 |
| 1 | COG3252 | Methenyltetrahydromethanopterin cyclohydrolase | MSM1723 |
| 2 | COG4054 | Methyl coenzyme M reductase, beta subunit | MSM0905, MSM1019 |
| 2 | COG4055 | Methyl coenzyme M reductase, subunit D | MSM0904, MSM1018 |
| 1 | COG4056 | Methyl coenzyme M reductase, subunit C | MSM1017 |
| 2 | COG4057 | Methyl coenzyme M reductase, gamma subunit | MSM0903, MSM1016 |
| 2 | COG4058 | Methyl coenzyme M reductase, alpha subunit | MSM0902, MSM1015 |
| 1 | COG4059 | Tetrahydromethanopterin S-methyltransferase, subunit E | MSM1014 |
| 1 | COG4060 | Tetrahydromethanopterin S-methyltransferase, subunit D | MSM1013 |
| 1 | COG4061 | Tetrahydromethanopterin S-methyltransferase, subunit C | MSM1012 |
| 1 | COG4062 | Tetrahydromethanopterin S-methyltransferase, subunit B | MSM1011 |
| 1 | COG4063 | Tetrahydromethanopterin S-methyltransferase, subunit A | MSM1010 |
| 1 | COG4064 | Tetrahydromethanopterin S-methyltransferase, subunit G | MSM1008 |
| 1 | COG4218 | Tetrahydromethanopterin S-methyltransferase, subunit F | MSM1009 |

Lipid Transport and Metabolism (I)

| | | | |
|---|---|---|---|
| 1 | COG0020 | Undecaprenyl pyrophosphate synthase | MSM0096 |
| 1 | COG0170 | Dolichol kinase | MSM0078 |
| 1 | COG0183 | Acetyl-CoA acetyltransferase | MSM1562 |
| 1 | COG0365 | Acyl-coenzyme A synthetases/AMP-(fatty) acid ligases | MSM0330 |
| 1 | COG0439 | Biotin carboxylase | MSM0765 |
| 2 | COG0558 | Phosphatidylglycerophosphate synthase | MSM0613, MSM1706 |
| 1 | COG0575 | CDP-diglyceride synthetase | MSM0850 |
| 1 | COG1183 | Phosphatidylserine synthase | MSM0982 |
| 2 | COG1211 | 4-diphosphocytidyl-2-methyl-D-erithritol synthase | MSM0377, MSM1542 |
| 1 | COG1250 | 3-hydroxyacyl-CoA dehydrogenase | MSM0965 |
| 1 | COG1257 | Hydroxymethylglutaryl-CoA reductase | MSM0227 |
| 1 | COG1260 | Myo-inositol-1-phosphate synthase | MSM0940 |
| 1 | COG1267 | Phosphatidylglycerophosphatase A and related proteins | MSM0934 |
| 1 | COG1577 | Mevalonate kinase | MSM1439 |
| 1 | COG1924 | Activator of 2-hydroxyglutaryl-CoA dehydratase (HSP70-class | MSM0810 |
| | | ATPase domain) | |
| 1 | COG2084 | | MSM0548 |
| 1 | COG3425 | 3-hydroxy-3-methylglutaryl CoA synthase | MSM1561 |

Inorganic Ion Transport and Metabolism (P)

| | | | |
|---|---|---|---|
| 1 | COG0003 | Oxyanion-translocating ATPase | MSM1170 |
| 1 | COG0004 | Ammonia permease | MSM0234 |
| 1 | COG0038 | Chloride channel protein EriC | MSM1721 |
| 1 | COG0053 | Predicted Co/Zn/Cd cation transporters | MSM0789 |
| 1 | COG0168 | Trk-type K+ transport systems, membrane components | MSM1095 |
| 1 | COG0226 | ABC-type phosphate transport system, periplasmic component | MSM0568 |
| 1 | COG0288 | Carbonic anhydrase | MSM1223 |
| 4 | COG0310 | ABC-type Co2+ transport system, permease component | MSM0583, MSM0584, MSM1488, |
| | | | MSM1618 |
| 1 | COG0370 | Fe2+ transport system protein B | MSM0589 |
| 1 | COG0474 | Cation transport ATPase | MSM0895 |
| 1 | COG0475 | Kef-type K+ transport systems, membrane components | MSM1186 |
| 1 | COG0530 | Ca2+/Na+ antiporter | MSM1027 |
| 1 | COG0569 | K+ transport systems, NAD-binding component | MSM1096 |
| 1 | COG0573 | ABC-type phosphate transport system, permease component | MSM0567 |
| 1 | COG0581 | ABC-type phosphate transport system, permease component | MSM0566 |
| 1 | COG0600 | ABC-type nitrate/sulfonate/bicarbonate transport system, permease | MSM0291 |
| | | component | |
| 1 | COG0609 | ABC-type Fe3+-siderophore transport system, permease | MSM1394 |
| | | component | |
| 1 | COG0614 | ABC-type Fe3+-hydroxamate transport system, periplasmic | MSM1393 |
| | | component | |
| 3 | COG0619 | ABC-type cobalt transport system, permease component CbiQ and | MSM0585, MSM0771, MSM1620 |
| | | related transporters | |
| 2 | COG0704 | Phosphate uptake regulator | MSM0564, MSM0569 |
| 1 | COG0715 | ABC-type nitrate/sulfonate/bicarbonate transport systems, | MSM1469 |
| | | periplasmic components | |
| 1 | COG0725 | ABC-type molybdate transport system, periplasmic component | MSM1609 |
| 1 | COG0798 | Arsenite efflux pump ACR3 and related permeases | MSM1078 |
| 1 | COG0855 | Polyphosphate kinase | MSM1424 |
| 1 | COG1006 | Multisubunit Na+/H+ antiporter, MnhC subunit | MSM1072 |
| 1 | COG1116 | ABC-type nitrate/sulfonate/bicarbonate transport system, ATPase | MSM0290 |
| | | component | |
| 1 | COG1117 | ABC-type phosphate transport system, ATPase component | MSM0565 |
| 1 | COG1118 | ABC-type sulfate/molybdate transport systems, ATPase component | MSM1611 |
| 2 | COG1122 | ABC-type cobalt transport system, ATPase component | MSM0586, MSM1621 |
| 1 | COG1230 | Co/Zn/Cd efflux system component | MSM1639 |
| 1 | COG1320 | Multisubunit Na+/H+ antiporter, MnhG subunit | MSM1074 |
| 1 | COG1348 | Nitrogenase subunit NifH (ATPase) | MSM1707 |
| 1 | COG1528 | Ferritin-like protein | MSM1712 |
| 1 | COG1563 | Predicted subunit of the Multisubunit Na+/H+ antiporter | MSM1073 |
| 1 | COG1824 | Permease, similar to cation transporters | MSM1275 |
| 1 | COG1863 | Multisubunit Na+/H+ antiporter, MnhE subunit | MSM1076 |
| 1 | COG1918 | Fe2+ transport system protein A | MSM0588 |
| 1 | COG1930 | ABC-type cobalt transport system, periplasmic component | MSM1619 |
| 2 | COG2111 | Multisubunit Na+/H+ antiporter, MnhB subunit | MSM1068, MSM1069 |
| 1 | COG2116 | Formate/nitrite family of transporters | MSM1403 |
| 1 | COG2212 | Multisubunit Na+/H+ antiporter, MnhF subunit | MSM1075 |
| 4 | COG2217 | Cation transport ATPase | MSM0293, MSM0960, MSM1127, |
| | | | MSM1153 |
| 1 | COG2608 | Copper chaperone | MSM0961 |
| 1 | COG3263 | NhaP-type Na+/H+ and K+/H+ antiporters with a unique C-terminal | MSM0618 |
| | | domain | |
| 1 | COG3420 | Nitrous oxidase accessory protein | MSM1397 |
| 1 | COG4149 | ABC-type molybdate transport system, permease component | MSM1610 |

Secondary Metabolites Biosynthesis, Transport and Catabolism (Q)

| | | | |
|---|---|---|---|
| 1 | COG1228 | Imidazolonepropionase and related amidohydrolases | MSM1154 |

General Function Prediction Only (R)

| | | | |
|---|---|---|---|
| 2 | COG0110 | Acetyltransferase (isoleucine patch superfamily) | MSM0189, MSM1600 |
| 2 | COG0312 | Predicted Zn-dependent proteases and their inactivated homologs | MSM0866, MSM0947 |
| 1 | COG0375 | Zn finger protein HypA/HybF (possibly regulating hydrogenase | MSM0108 |
| | | expression) | |
| 1 | COG0388 | Predicted amidohydrolase | MSM0500 |
| 1 | COG0433 | Predicted ATPase | MSM0122 |
| 1 | COG0446 | Uncharacterized NAD(FAD)-dependent dehydrogenases | MSM0046 |
| 2 | COG0456 | Acetyltransferases | MSM0893, MSM1104 |
| 11 | COG0457 | FOG: TPR repeat | MSM0530, MSM0651, MSM0914, |
| | | | MSM1449, MSM1451, MSM1740, |
| | | | MSM1766, MSM1776, MSM1786, |
| | | | MSM1787, MSM1788 |
| 2 | COG0491 | Zn-dependent hydrolases, including glyoxylases | MSM0421, MSM1097 |
| 1 | COG0496 | Predicted acid phosphatase | MSM1218 |
| 2 | COG0517 | FOG: CBS domain | MSM0175, MSM1102 |
| 4 | COG0535 | Predicted Fe—S oxidoreductases | MSM0663, MSM0808, MSM1301, |
| | | | MSM1497 |
| 1 | COG0561 | Predicted hydrolases of the HAD superfamily | MSM0946 |
| 1 | COG0595 | Predicted hydrolase of the metallo-beta-lactamase superfamily | MSM1442 |
| 1 | COG0603 | Predicted PP-loop superfamily ATPase | MSM0936 |
| 1 | COG0613 | Predicted metal-dependent phosphoesterases (PHP family) | MSM1244 |
| 1 | COG0622 | Predicted phosphoesterase | MSM0507 |
| 1 | COG0627 | Predicted esterase | MSM0149 |
| 1 | COG0628 | Predicted permease | MSM1042 |
| 1 | COG0641 | Arylsulfatase regulator (Fe—S oxidoreductase) | MSM1606 |
| 5 | COG0655 | Multimeric flavodoxin WrbA | MSM0267, MSM0664, MSM0923, |
| | | | MSM1209, MSM1727 |
| 1 | COG0661 | Predicted unusual protein kinase | MSM0525 |
| 1 | COG0663 | Carbonic anhydrases/acetyltransferases, isoleucine patch | MSM0654 |
| | | superfamily | |
| 1 | COG0666 | FOG: Ankyrin repeat | MSM0266 |
| 1 | COG0673 | Predicted dehydrogenases and related proteins | MSM0882 |
| 1 | COG0679 | Predicted permeases | MSM1334 |
| 1 | COG0714 | MoxR-like ATPases | MSM0555 |
| 1 | COG0730 | Predicted permeases | MSM0420 |
| 3 | COG0733 | Na+-dependent transporters of the SNF family | MSM0699, MSM1531, MSM1532 |
| 1 | COG0824 | Predicted thioesterase | MSM0133 |
| 1 | COG1011 | Predicted hydrolase (HAD superfamily) | MSM1480 |
| 1 | COG1019 | Predicted nucleotidyltransferase | MSM0785 |
| 1 | COG1078 | HD superfamily phosphohydrolases | MSM0236 |
| 1 | COG1084 | Predicted GTPase | MSM0869 |
| 1 | COG1094 | Predicted RNA-binding protein (contains KH domains) | MSM0954 |
| 1 | COG1099 | Predicted metal-dependent hydrolases with the TIM-barrel fold | MSM0405 |
| 3 | COG1123 | ATPase components of various ABC-type transport systems, | MSM0770, MSM0971, MSM1698 |
| | | contain duplicated ATPase | |
| 2 | COG1163 | Predicted GTPase | MSM0714, MSM0715 |
| 1 | COG1201 | Lhr-like helicases | MSM0502 |
| 1 | COG1202 | Superfamily II helicase, archaea-specific | MSM1583 |
| 1 | COG1203 | Predicted helicases | MSM0166 |
| 1 | COG1204 | Superfamily II helicase | MSM0839 |
| 1 | COG1205 | Distinct helicase family with a unique C-terminal domain including a | MSM0112 |
| | | metal-binding cysteine cluster | |
| 5 | COG1216 | Predicted glycosyltransferases | MSM1321, MSM1329, MSM1330, |
| | | | MSM1503, MSM1507 |
| 1 | COG1223 | Predicted ATPase (AAA+ superfamily) | MSM0966 |
| 1 | COG1234 | Metal-dependent hydrolases of the beta-lactamase superfamily III | MSM0492 |
| 1 | COG1235 | Metal-dependent hydrolases of the beta-lactamase superfamily I | MSM1473 |
| 1 | COG1244 | Predicted Fe—S oxidoreductase | MSM0544 |
| 1 | COG1245 | Predicted ATPase, RNase L inhibitor (RLI) homolog | MSM0607 |
| 1 | COG1253 | Hemolysins and related proteins containing CBS domains | MSM1026 |
| 4 | COG1266 | Predicted metal-dependent membrane protease | MSM0292, MSM0803, MSM1148, |
| | | | MSM1180 |
| 1 | COG1268 | Uncharacterized conserved protein | MSM0429 |
| 2 | COG1277 | ABC-type transport system involved in multi-copper enzyme | MSM0594, MSM0595 |
| | | maturation, permease component | |
| 1 | COG1287 | Uncharacterized membrane protein, required for N-linked | MSM0716 |
| | | glycosylation | |
| 1 | COG1310 | Predicted metal-dependent protease of the PAD1/JAB1 | MSM0462 |
| | | superfamily | |
| 2 | COG1323 | Predicted nucleotidyltransferase | MSM0547, MSM0994 |
| 1 | COG1326 | Uncharacterized archaeal Zn-finger protein | MSM0846 |
| 2 | COG1342 | Predicted DNA-binding proteins | MSM0207, MSM0208 |
| 1 | COG1350 | Predicted alternative tryptophan synthase beta-subunit (paralog of | MSM1242 |
| | | TrpB) | |
| 1 | COG1355 | Predicted dioxygenase | MSM1438 |
| 1 | COG1365 | Predicted ATPase (PP-loop superfamily) | MSM0190 |
| 9 | COG1373 | Predicted ATPase (AAA+ superfamily) | MSM0061, MSM0280, MSM0680, |
| | | | MSM1197, MSM1278, MSM1527, |
| | | | MSM1789, MSM1790, MSM1795 |
| 1 | COG1402 | Uncharacterized protein, putative amidase | MSM0184 |
| 2 | COG1408 | Predicted phosphohydrolases | MSM0964, MSM1165 |
| 1 | COG1409 | Predicted phosphohydrolases | MSM0383 |
| 1 | COG1411 | Uncharacterized protein related to proFAR isomerase (HisA) | MSM1636 |
| 1 | COG1412 | Uncharacterized proteins of PilT N-term./Vapc superfamily | MSM0199 |
| 1 | COG1418 | Predicted HD superfamily hydrolase | MSM0632 |
| 1 | COG1439 | Predicted nucleic acid-binding protein, consists of a PIN domain | MSM0816 |
| | | and a Znribbon module | |
| 4 | COG1453 | Predicted oxidoreductases of the aldo/keto reductase family | MSM0148, MSM0728, MSM1450, |
| | | | MSM1608 |
| 1 | COG1489 | DNA-binding protein, stimulates sugar fermentation | MSM1090 |
| 1 | COG1537 | Predicted RNA-binding proteins | MSM0640 |
| 1 | COG1545 | Predicted nucleic-acid-binding protein containing a Zn-ribbon | MSM1279 |
| 2 | COG1571 | Predicted DNA-binding protein containing a Zn-ribbon domain | MSM0452, MSM1295 |
| 1 | COG1606 | ATP-utilizing enzymes of the PP-loop superfamily | MSM0482 |
| 1 | COG1608 | Predicted archaeal kinase | MSM1440 |
| 1 | COG1611 | Predicted Rossmann fold nucleotide-binding protein | MSM0004 |
| 1 | COG1634 | Uncharacterized Rossmann fold enzyme | MSM0672 |
| 1 | COG1646 | Predicted phosphate-binding enzymes, TIM-barrel fold | MSM0124 |
| 2 | COG1672 | Predicted ATPase (AAA+ superfamily) | MSM1196, MSM1646 |
| 1 | COG1691 | NCAIR mutase (PurE)-related proteins | MSM1105 |
| 1 | COG1707 | ACT domain-containing protein | MSM1060 |
| 1 | COG1759 | ATP-utilizing enzymes of ATP-grasp superfamily (probably | MSM0506 |
| | | carboligases) | |
| 1 | COG1779 | C4-type Zn-finger protein | MSM0409 |
| 1 | COG1782 | Predicted metal-dependent RNase, consists of a metallo-beta- | MSM1038 |
| | | lactamase domain and an RNA-binding KH domain | |
| 1 | COG1821 | Predicted ATP-utilizing enzyme (ATP-grasp superfamily) | MSM0852 |
| 1 | COG1829 | Predicted archaeal kinase (sugar kinase superfamily) | MSM0060 |
| 1 | COG1855 | ATPase (PilT family) | MSM1183 |
| 1 | COG1878 | Predicted metal-dependent hydrolase | MSM0827 |
| 1 | COG1907 | Predicted archaeal sugar kinases | MSM0848 |
| 1 | COG1942 | Uncharacterized protein, 4-oxalocrotonate tautomerase homolog | MSM0688 |
| 1 | COG1964 | Predicted Fe—S oxidoreductases | MSM0849 |
| 1 | COG1988 | Predicted membrane-bound metal-dependent hydrolases | MSM1079 |
| 1 | COG1994 | Zn-dependent proteases | MSM0479 |
| 2 | COG2005 | N-terminal domain of molybdenum-binding protein | MSM0131, MSM1207 |
| 1 | COG2047 | Uncharacterized protein (ATP-grasp superfamily) | MSM1131 |
| 1 | COG2054 | Uncharacterized archaeal kinase related to aspartokinases, | MSM0604 |
| | | uridylate kinases | |
| 1 | COG2068 | Uncharacterized MobA-related protein | MSM0116 |
| 1 | COG2079 | Uncharacterized protein involved in propionate catabolism | MSM0449 |
| 1 | COG2081 | Predicted flavoproteins | MSM1235 |
| 1 | COG2085 | Predicted dinucleotide-binding enzymes | MSM0049 |
| 1 | COG2102 | Predicted ATPases of PP-loop superfamily | MSM0142 |
| 1 | COG2118 | DNA-binding protein | MSM0708 |
| 1 | COG2129 | Predicted phosphoesterases, related to the lcc protein | MSM0792 |
| 1 | COG2150 | Predicted regulator of amino acid metabolism, contains ACT | MSM0635 |
| | | domain | |
| 1 | COG2151 | Predicted metal-sulfur cluster biosynthetic enzyme | MSM0634 |
| 1 | COG2220 | Predicted Zn-dependent hydrolases of the beta-lactamase fold | MSM0779 |
| 1 | COG2232 | Predicted ATP-dependent carboligase related to biotin carboxylase | MSM0431 |
| 3 | COG2244 | Membrane protein involved in the export of O-antigen and teichoic | MSM1208, MSM1559, MSM1560 |
| | | acid | |
| 1 | COG2252 | Permeases | MSM1736 |
| 1 | COG2403 | Predicted GTPase | MSM0091 |
| 1 | COG2405 | Predicted nucleic acid-binding protein, contains PIN domain | MSM1530 |
| 1 | COG2517 | Predicted RNA-binding protein containing a C-terminal EMAP | MSM0466 |
| | | domain | |
| 2 | COG2520 | Predicted methyltransferase | MSM0802, MSM1036 |
| 1 | COG2522 | Predicted transcriptional regulator | MSM0269 |
| 3 | COG3291 | FOG: PKD repeat | MSM0281, MSM1716, MSM1735 |
| 1 | COG3442 | Predicted glutamine amidotransferase | MSM1138 |
| 1 | COG3552 | Protein containing von Willebrand factor type A (vWA) domain | MSM0554 |
| 1 | COG3608 | Predicted deacylase | MSM1080 |
| 1 | COG3894 | Uncharacterized metal-binding protein | MSM0517 |
| 1 | COG3942 | Surface antigen | MSM0921 |
| 1 | COG3943 | Virulence protein | MSM1645 |
| 1 | COG4002 | Predicted phosphotransacetylase | MSM0095 |
| 1 | COG4015 | Predicted dinucleotide-utilizing enzyme of the ThiF/HesA family | MSM0577 |
| 1 | COG4026 | Uncharacterized protein containing TOPRIM domain, potential | MSM1703 |
| | | nuclease | |
| 2 | COG4032 | Predicted thiamine-pyrophosphate-binding protein | MSM0080, MSM0081 |
| 1 | COG4052 | Uncharacterized protein related to methyl coenzyme M reductase | MSM1021 |
| | | subunit C | |
| 1 | COG4076 | Predicted RNA methylase | MSM0363 |
| 1 | COG4085 | Predicted RNA-binding protein, contains TRAM domain | MSM0647 |
| 1 | COG4087 | Soluble P-type ATPase | MSM1252 |
| 1 | COG4277 | Predicted DNA-binding protein with the Helix-hairpin-helix motif | MSM1239 |
| 2 | COG4747 | ACT domain-containing protein | MSM0388, MSM1713 |
| 1 | COG4801 | Predicted acyltransferase | MSM1385 |
| 1 | COG4827 | Predicted transporter | MSM1717 |
| 1 | COG5012 | Predicted cobalamin binding protein | MSM0516 |
| 3 | COG5271 | AAA ATPase containing von Willebrand factor type A (vWA) | MSM0993, MSM1240, MSM1454 |
| | | domain | |
| 1 | COG5362 | Phage-related terminase | MSM1671 |
| 1 | COG5518 | Bacteriophage capsid portal protein | MSM1672 |
| 2 | COG5643 | Protein containing a metal-binding domain shared with | MSM1489, MSM1491 |
| | | formylmethanofuran dehydrogenase subunit E | |

Function Unknown (S)

| | | | |
|---|---|---|---|
| 1 | COG0011 | Uncharacterized conserved protein | MSM1029 |
| 2 | COG0028 | | MSM0686, MSM1225 |
| 1 | COG0059 | | MSM1222 |
| 1 | COG0111 | | MSM0457 |
| 1 | COG0147 | | MSM1146 |
| 1 | COG0248 | | MSM1423 |
| 2 | COG0318 | | MSM0025, MSM0374 |
| 1 | COG0327 | Uncharacterized conserved protein | MSM0576 |
| 1 | COG0378 | | MSM0107 |
| 1 | COG0391 | Uncharacterized conserved protein | MSM0974 |
| 1 | COG0392 | Predicted integral membrane protein | MSM1094 |
| 2 | COG0393 | Uncharacterized conserved protein | MSM0418, MSM0456 |
| 1 | COG0432 | Uncharacterized conserved protein | MSM0279 |
| 1 | COG0444 | | MSM0303 |
| 1 | COG0451 | | MSM0327 |
| 2 | COG0458 | | MSM0361, MSM0488 |
| 1 | COG0462 | | MSM1577 |
| 2 | COG0473 | | MSM0373, MSM1298 |
| 2 | COG0477 | | MSM0772, MSM1210 |
| 2 | COG0500 | | MSM0028, MSM1510 |
| 1 | COG0505 | | MSM0489 |
| 1 | COG0512 | | MSM1145 |
| 1 | COG0513 | | MSM1498 |
| 1 | COG0543 | | MSM1043 |
| 1 | COG0585 | Uncharacterized conserved protein | MSM1156 |
| 1 | COG0591 | | MSM0386 |
| 1 | COG0599 | Uncharacterized homolog of gamma-carboxymuconolactone | MSM0296 |
| | | decarboxylase subunit | |
| 1 | COG0601 | | MSM0301 |
| 2 | COG0615 | | MSM0859, MSM1514 |
| 1 | COG1028 | | MSM1731 |
| 2 | COG1061 | | MSM0690, MSM0695 |
| 1 | COG1063 | | MSM0376 |
| 1 | COG1086 | | MSM1535 |
| 1 | COG1120 | | MSM1395 |
| 1 | COG1124 | | MSM0304 |
| 2 | COG1134 | | MSM1326, MSM1592 |
| 1 | COG1173 | | MSM0302 |
| 1 | COG1199 | | MSM1352 |
| 1 | COG1208 | | MSM0655 |
| 1 | COG1243 | | MSM0842 |
| 1 | COG1255 | Uncharacterized protein conserved in archaea | MSM0894 |
| 2 | COG1300 | Uncharacterized membrane protein | MSM0215, MSM1526 |
| 1 | COG1303 | Uncharacterized protein conserved in archaea | MSM0932 |
| 1 | COG1339 | | MSM1257 |
| 1 | COG1359 | Uncharacterized conserved protein | MSM1378 |
| 1 | COG1371 | Uncharacterized conserved protein | MSM0668 |
| 1 | COG1379 | Uncharacterized conserved protein | MSM1129 |
| 1 | COG1387 | | MSM0063 |
| 1 | COG1415 | Uncharacterized conserved protein | MSM0931 |
| 1 | COG1422 | Predicted membrane protein | MSM0736 |
| 1 | COG1430 | Uncharacterized conserved protein | MSM1339 |
| 1 | COG1460 | Uncharacterized protein conserved in archaea | MSM1376 |
| 1 | COG1469 | Uncharacterized conserved protein | MSM1033 |
| 2 | COG1474 | | MSM0671, MSM1264 |
| 1 | COG1478 | Uncharacterized conserved protein | MSM0975 |
| 1 | COG1511 | Predicted membrane protein | MSM0093 |
| 2 | COG1520 | FOG: WD40-like repeat | MSM1247, MSM1567 |
| 1 | COG1548 | | MSM0851 |
| 1 | COG1578 | Uncharacterized conserved protein | MSM0551 |
| 1 | COG1602 | Uncharacterized conserved protein | MSM0346 |
| 2 | COG1617 | Uncharacterized conserved protein | MSM0348, MSM0349 |
| 1 | COG1627 | Uncharacterized protein conserved in archaea | MSM0983 |
| 1 | COG1630 | Uncharacterized protein conserved in archaea | MSM0123 |
| 1 | COG1641 | Uncharacterized conserved protein | MSM0935 |
| 1 | COG1665 | Uncharacterized protein conserved in archaea | MSM1058 |
| 1 | COG1679 | Uncharacterized conserved protein | MSM1192 |
| 1 | COG1685 | | MSM0835 |
| 1 | COG1690 | Uncharacterized conserved protein | MSM0666 |
| 1 | COG1693 | Uncharacterized protein conserved in archaea | MSM1417 |
| 1 | COG1698 | Uncharacterized protein conserved in archaea | MSM1268 |
| 1 | COG1701 | Uncharacterized protein conserved in archaea | MSM0140 |
| 2 | COG1704 | Uncharacterized conserved protein | MSM0660, MSM1422 |
| 1 | COG1710 | Uncharacterized protein conserved in archaea | MSM0069 |
| 1 | COG1711 | Uncharacterized protein conserved in archaea | MSM1136 |
| 1 | COG1714 | Predicted membrane protein/domain | MSM1493 |
| 1 | COG1718 | | MSM0952 |
| 1 | COG1720 | Uncharacterized conserved protein | MSM0132 |
| 2 | COG1738 | Uncharacterized conserved protein | MSM0646, MSM1382 |
| 1 | COG1739 | Uncharacterized conserved protein | MSM0186 |
| 1 | COG1751 | Uncharacterized conserved protein | MSM0628 |
| 1 | COG1771 | Uncharacterized protein conserved in archaea | MSM0070 |
| 1 | COG1784 | Predicted membrane protein | MSM0599 |
| 1 | COG1786 | Uncharacterized conserved protein | MSM1155 |
| 1 | COG1795 | Uncharacterized conserved protein | MSM1213 |
| 1 | COG1809 | Uncharacterized conserved protein | MSM0086 |
| 1 | COG1817 | Uncharacterized protein conserved in archaea | MSM0106 |
| 2 | COG1822 | Predicted archaeal membrane protein | MSM0581, MSM1216 |
| 1 | COG1836 | Predicted membrane protein | MSM0659 |
| 1 | COG1844 | Uncharacterized protein conserved in archaea | MSM0356 |
| 1 | COG1849 | Uncharacterized protein conserved in archaea | MSM0614 |
| 2 | COG1852 | Uncharacterized conserved protein | MSM0225, MSM0649 |
| 1 | COG1860 | Uncharacterized protein conserved in archaea | MSM0285 |
| 1 | COG1865 | Uncharacterized conserved protein | MSM0825 |
| 1 | COG1872 | Uncharacterized conserved protein | MSM1603 |
| 4 | COG1873 | Uncharacterized conserved protein | MSM0465, MSM0822, MSM0841, |
| | | | MSM1004 |
| 1 | COG1891 | Uncharacterized protein conserved in archaea | MSM1628 |
| 1 | COG1909 | Uncharacterized protein conserved in archaea | MSM0195 |
| 1 | COG1915 | Uncharacterized conserved protein | MSM0875 |
| 1 | COG1916 | Uncharacterized homolog of PrgY (pheromone shutdown protein) | MSM1024 |
| 1 | COG1917 | Uncharacterized conserved protein, contains double-stranded | MSM1447 |
| | | beta-helix domain | |
| 1 | COG1920 | Uncharacterized conserved protein | MSM0288 |
| 1 | COG1937 | Uncharacterized protein conserved in bacteria | MSM0959 |
| 1 | COG1944 | Uncharacterized conserved protein | MSM0480 |
| 1 | COG1945 | Uncharacterized conserved protein | MSM0878 |
| 1 | COG1950 | Predicted membrane protein | MSM1166 |
| 1 | COG1971 | Predicted membrane protein | MSM0030 |
| 1 | COG1990 | Uncharacterized conserved protein | MSM0605 |
| 1 | COG1991 | Uncharacterized conserved protein | MSM0145 |
| 1 | COG2029 | Uncharacterized conserved protein | MSM1057 |
| 1 | COG2035 | Predicted membrane protein | MSM1582 |
| 1 | COG2042 | Uncharacterized conserved protein | MSM0126 |
| 1 | COG2043 | Uncharacterized protein conserved in archaea | MSM0115 |
| 1 | COG2078 | Uncharacterized conserved protein | MSM0867 |
| 1 | COG2090 | Uncharacterized protein conserved in archaea | MSM1591 |
| 1 | COG2098 | Uncharacterized protein conserved in archaea | MSM0985 |
| 1 | COG2106 | Uncharacterized conserved protein | MSM0763 |
| 1 | COG2122 | Uncharacterized conserved protein | MSM0088 |
| 1 | COG2136 | | MSM1632 |
| 2 | COG2138 | Uncharacterized conserved protein | MSM1280, MSM1281 |
| 1 | COG2246 | Predicted membrane protein | MSM1289 |
| 2 | COG2314 | Predicted membrane protein | MSM0109, MSM1739 |
| 2 | COG2364 | Predicted membrane protein | MSM0673, MSM0676 |
| 1 | COG2429 | Uncharacterized conserved protein | MSM0973 |
| 1 | COG2450 | Uncharacterized conserved protein | MSM0406 |
| 1 | COG2456 | Uncharacterized conserved protein | MSM1624 |
| 1 | COG2457 | Uncharacterized conserved protein | MSM0873 |
| 1 | COG2892 | Uncharacterized protein conserved in archaea | MSM1633 |
| 1 | COG3273 | Uncharacterized conserved protein | MSM1274 |
| 2 | COG3274 | Uncharacterized protein conserved in bacteria | MSM1370, MSM1556 |
| 1 | COG3356 | Predicted membrane protein | MSM0776 |
| 1 | COG3367 | Uncharacterized conserved protein | MSM0407 |
| 1 | COG3482 | Uncharacterized conserved protein | MSM0481 |
| 1 | COG3543 | Uncharacterized conserved protein | MSM0430 |
| 3 | COG3548 | Predicted integral membrane protein | MSM0468, MSM0469, MSM1205 |
| 1 | COG3586 | Uncharacterized conserved protein | MSM1741 |
| 1 | COG3815 | Predicted membrane protein | MSM1770 |
| 1 | COG3874 | Uncharacterized conserved protein | MSM0683 |
| 1 | COG3976 | Uncharacterized protein conserved in bacteria | MSM1637 |
| 1 | COG4009 | Uncharacterized protein conserved in archaea | MSM0794 |
| 1 | COG4010 | Uncharacterized protein conserved in archaea | MSM0793 |
| 1 | COG4012 | Uncharacterized protein conserved in archaea | MSM1243 |
| 1 | COG4014 | Uncharacterized protein conserved in archaea | MSM0840 |
| 1 | COG4016 | Uncharacterized protein conserved in archaea | MSM0578 |
| 1 | COG4017 | Uncharacterized protein conserved in archaea | MSM0575 |
| 1 | COG4018 | Uncharacterized protein conserved in archaea | MSM0571 |
| 1 | COG4019 | Uncharacterized protein conserved in archaea | MSM0574 |
| 1 | COG4020 | Uncharacterized protein conserved in archaea | MSM1221 |
| 1 | COG4021 | Uncharacterized conserved protein | MSM0463 |
| 1 | COG4022 | Uncharacterized protein conserved in archaea | MSM0643 |
| 1 | COG4029 | Uncharacterized protein conserved in archaea | MSM0812 |
| 1 | COG4030 | Uncharacterized protein conserved in archaea | MSM0309 |
| 1 | COG4033 | Uncharacterized protein conserved in archaea | MSM0103 |
| 1 | COG4035 | Predicted membrane protein | MSM0315 |
| 1 | COG4036 | Predicted membrane protein | MSM0320 |
| 1 | COG4037 | Predicted membrane protein | MSM0321 |
| 1 | COG4038 | Predicted membrane protein | MSM0322 |
| 1 | COG4039 | Predicted membrane protein | MSM0323 |
| 1 | COG4040 | Predicted membrane protein | MSM0324 |
| 1 | COG4041 | Predicted membrane protein | MSM0325 |
| 1 | COG4042 | Predicted membrane protein | MSM0326 |
| 2 | COG4050 | Uncharacterized protein conserved in archaea | MSM0811, MSM1130 |
| 1 | COG4051 | Uncharacterized protein conserved in archaea | MSM0809 |
| 1 | COG4053 | Uncharacterized protein conserved in archaea | MSM0229 |
| 1 | COG4065 | Uncharacterized protein conserved in archaea | MSM1006 |
| 2 | COG4066 | Uncharacterized protein conserved in archaea | MSM0064, MSM0367 |
| 1 | COG4068 | Uncharacterized protein containing a Zn-ribbon | MSM0417 |
| 1 | COG4069 | Uncharacterized protein conserved in archaea | MSM0815 |
| 1 | COG4071 | Uncharacterized protein conserved in archaea | MSM0630 |
| 1 | COG4073 | Uncharacterized protein conserved in archaea | MSM0726 |
| 1 | COG4077 | Uncharacterized protein conserved in archaea | MSM1034 |
| 1 | COG4078 | Predicted membrane protein | MSM0319 |
| 1 | COG4079 | Uncharacterized protein conserved in archaea | MSM1472 |
| 1 | COG4081 | Uncharacterized protein conserved in archaea | MSM0104 |
| 1 | COG4084 | Uncharacterized protein conserved in archaea | MSM0314 |
| 1 | COG4121 | Uncharacterized conserved protein | MSM1555 |
| 1 | COG4289 | Uncharacterized protein conserved in bacteria | MSM1302 |
| 1 | COG4635 | | MSM1262 |
| 3 | COG4713 | Predicted membrane protein | MSM0521, MSM1291, MSM1444 |
| 2 | COG4744 | Uncharacterized conserved protein | MSM1402, MSM1719 |
| 1 | COG4883 | Uncharacterized protein conserved in archaea | MSM1086 |
| 1 | COG4907 | Predicted membrane protein | MSM1421 |
| 1 | COG5015 | Uncharacterized conserved protein | MSM0863 |
| 1 | COG5305 | Predicted membrane protein | MSM1288 |
| 1 | COG5423 | Predicted metal-binding protein | MSM0050 |
| 1 | COG5440 | Uncharacterized conserved protein | MSM1265 |
| 4 | COG5464 | Uncharacterized conserved protein | MSM0067, MSM0681, MSM1765, |
| | | | MSM1785 |

TABLE 10
Glycosyltransferases (GT) in M. smithii and M. stadtmanae proteomes
classified according to Carbohydrate Active enZyme (CAZy) database
CAZy GT family Protein Annotation

| | | | |
|---|---|---|---|
| M. smithii | GT1 | MSM0423* | glycosyltransferase (modular protein with two domains distantly related to |
| | | | glycosyltransferases), GT2/GT1 families [CAZy] |
| | GT2 | MSM0423* | glycosyltransferase (modular protein with two domains distantly related to |
| | | | glycosyltransferases), GT2/GT1 families [CAZy] |
| | | MSM1290 | glycosyltransferase (related to beta-glycosyltransferases), GT2 family |
| | | | [CAZy] |
| | | MSM1294 | glycosyltransferase (related to beta-glycosyltransferases), GT2 family |
| | | | [CAZy] |
| | | MSM1297 | glycosyltransferase (related to beta-glycosyltransferases), GT2 family |
| | | | [CAZy] |
| | | MSM1310 | glycosyltransferase (related to beta-glycosyltransferases), GT2 family |
| | | | [CAZy] |
| | | MSM1311 | glycosyltransferase (related to beta-glycosyltransferases), GT2 family |
| | | | [CAZy] |
| | | MSM1312 | glycosyltransferase (related to beta-glycosyltransferases), GT2 family |
| | | | [CAZy] |
| | | MSM1316 | glycosyltransferase (related to beta-glycosyltransferases), GT2 family |
| | | | [CAZy] |
| | | MSM1321 | glycosyltransferase (related to beta-glycosyltransferases), GT2 family |
| | | | [CAZy] |
| | | MSM1323 | glycosyltransferase (related to beta-glycosyltransferases), GT2 family |
| | | | [CAZy] |
| | | MSM1324 | glycosyltransferase (related to beta-glycosyltransferases), GT2 family |
| | | | [CAZy] |
| | | MSM1328 | glycosyltransferase (related to beta-glycosyltransferases), GT2 family |
| | | | [CAZy] |
| | | MSM1329 | glycosyltransferase (related to beta-glycosyltransferases), GT2 family |
| | | | [CAZy] |
| | | MSM1330 | glycosyltransferase (related to beta-glycosyltransferases), GT2 family |
| | | | [CAZy] |
| | | MSM1503 | glycosyltransferase (related to beta-glycosyltransferases), GT2 family |
| | | | [CAZy] |
| | | MSM1507 | glycosyltransferase (related to beta-glycosyltransferases), GT2 family |
| | | | [CAZy] |
| | | MSM1545 | glycosyltransferase (related to beta-glycosyltransferases), GT2 family |
| | | | [CAZy] |
| | | MSM1594 | glycosyltransferase (modular protein with two N-terminal beta- |
| | | | glycosyltransferaserelated domains and C-terminal |
| | | | glycerophosphotransferase-related domain), GT2 families [CAZy] |
| | | MSM1602 | glycosyltransferase (modular protein with N-terminal beta- |
| | | | glycosyltransferase-related domain and C-terminal |
| | | | glycerophosphotransferase-related domain), GT2 family [CAZy] |
| | | MSM1623 | glycosyltransferase (related to beta-glycosyltransferases), GT2 family |
| | | | [CAZy] |
| | | MSM1627 | glycosyltransferase (related to bactoprenol beta-glucosyltransferase), GT2 |
| | | | family [CAZy] |
| | GT4 | MSM0836 | related to alpha-glycosyltransferases, GT4 family [CAZy] |
| | | MSM1313 | distantly related to glycosyltransferases, GT4 family [CAZy] |
| | | MSM1317 | distantly related to glycosyltransferases, GT4 family [CAZy] |
| | | MSM1322 | distantly related to alpha-glycosyltransferases, GT4 family [CAZy] |
| | GT66 | MSM0716 | glycosyltransferase (distantly related to oligosaccharyltransferases), STT3 |
| | | | subunit, GT66 family [CAZy] |
| M. stadtmanae | GT1 | Msp_0515 | partially conserved hypothetical protein |
| | | Msp_0645 | predicted glycosyltransferase |
| | GT2 | Msp_0042** | predicted glycosyltransferase |
| | | Msp_0045 | predicted glycosyltransferase |
| | | Msp_0054 | predicted glycosyltransferase |
| | | Msp_0203 | predicted glycosyltransferase |
| | | Msp_0206 | predicted glycosyltransferase |
| | | Msp_0207 | predicted glycosyltransferase |
| | | Msp_0212 | predicted glycosyltransferase |
| | | Msp_0215 | predicted glycosyltransferase |
| | | Msp_0218 | predicted glycosyltransferase |
| | | Msp_0220 | predicted glycosyltransferase |
| | | Msp_0441 | predicted glycosyltransferase |
| | | Msp_0442 | predicted glycosyltransferase |
| | | Msp_0492 | predicted glycosyltransferase |
| | | Msp_0493 | predicted glycosyltransferase |
| | | Msp_0495 | predicted glycosyltransferase |
| | | Msp_0496 | predicted glycosyltransferase |
| | | Msp_0500 | predicted glycosyltransferase |
| | | Msp_0538 | predicted glycosyltransferase |
| | | Msp_0541 | predicted glycosyltransferase |
| | | Msp_0645 | predicted glycosyltransferase |
| | | Msp_0989 | predicted glycosyltransferase |
| | | Msp_1087 | predicted glycosyltransferase |
| | | Msp_1481 | conserved hypothetical membrane-spanning protein |
| | | Msp_1540 | partially conserved hypothetical protein |
| | GT4 | Msp_0039 | predicted glycosyltransferase |
| | | Msp_0044 | predicted glycosyltransferase |
| | | Msp_0049 | predicted glycosyltransferase |
| | | Msp_0051 | predicted glycosyltransferase |
| | | Msp_0052 | predicted glycosyltransferase |
| | | Msp_0053 | predicted glycosyltransferase |
| | | Msp_0055 | predicted glycosyltransferase |
| | | Msp_0056 | predicted glycosyltransferase |
| | | Msp_0057 | predicted glycosyltransferase |
| | | Msp_0101 | predicted glycosyltransferase |
| | | Msp_0492 | predicted glycosyltransferase |
| | | Msp_0991 | predicted glycosyltransferase |
| | GT66 | Msp_0368 | conserved hypothetical membrane-spanning protein |

*modular protein
**probable fragment

TABLE 11
qRT-PCR analyses of M. smithii transcription in vivo in
the presence or absence of B. thetaiotaomicron VPI-5482

| | | | | |
|---|---|---|---|---|
| | | Fold | | P- |
| Gene | Annotation | Difference¹ | SEM | value |

CELL SURFACE

| | | | | |
|---|---|---|---|---|
| MSM1539 | sialic acid synthase, NeuB | 2.30 | 0.79 | 0.23 |
| MSM1305 | adhesin-like protein | 1.84 | 0.22 | 0.04 |
| MSM1112 | adhesin-like protein | 1.31 | 0.08 | 0.39 |
| MSM1113 | adhesin-like protein | 0.93 | 0.09 | 0.85 |
| MSM0411 | adhesin-like protein | 0.65 | 0.02 | 0.0006 |
| MSM1399 | adhesin-like protein | 0.60 | 0.05 | 0.0008 |
| MSM0995 | adhesin-like protein | 0.55 | 0.03 | 0.0009 |
| MSM1534 | adhesin-like protein | 0.52 | 0.10 | 0.03 |

METHANOGENESIS

| | | | | |
|---|---|---|---|---|
| MSM1381 | putative alcohol dehydrogenase, Adh | 2.31 | 0.62 | 0.003 |
| MSM0049 | F420-dependent NADP reductase, Fno | 3.75 | 0.41 | 0.006 |
| MSM0515 | methanol: cobalamin methyltransferase, MtaB | 2.37 | 0.32 | 0.01 |
| MSM0848 | ribofuranosylaminobenzene 5′-phosphate synthase, RfaS | 4.62 | 0.85 | 0.01 |

CARBON ASSIMILATION

| | | | | |
|---|---|---|---|---|
| MSM0330 | acetyl-CoA synthetase, Acs | 1.02 | 0.36 | 0.76 |
| MSM0228 | succinyl-CoA synthetase, alpha subunit, Suc | 1.33 | 0.24 | 0.31 |
| MSM0560 | pyruvate: ferredoxin oxidoreductase, beta subunit, Por | 4.92 | 0.60 | 0.0006 |
| MSM0988 | phosphoenolpyruvate synthase, PpsA | 2.72 | 0.42 | 0.002 |
| MSM0654 | carbonic anhydrase, Cab | 1.69 | 0.10 | 0.005 |
| MSM0991 | bicarbonate ABC transporter, substrate-binding component, | 0.55 | 0.05 | 0.005 |
| MSM0291 | bicarbonate ABC transporter, permease component, BtcB | 0.45 | 0.04 | 0.0006 |

NITROGEN ASSIMILATION

| | | | | |
|---|---|---|---|---|
| MSM0234 | ammonium transporter, AmtB | 2.88 | 0.24 | 0.0002 |
| MSM0888 | glutamate dehydrogenase, AdhA | 2.55 | 0.72 | 0.05 |
| MSM0027 | glutamate synthase, AltB | 2.35 | 0.64 | 0.006 |
| MSM0368 | glutamate synthase (NADPH), alpha subunit, GltA | 2.89 | 0.60 | 0.008 |
| MSM1418 | glutamine synthetase, GlnA | 19.06 | 5.35 | 0.0005 |

LIPID METABOLISM

| | | | | |
|---|---|---|---|---|
| MSM0227 | Hydroxymethylglutaryl-CoA (HMG-CoA) reductase, HmgA | 0.78 | 0.11 | 0.15 |

¹M. smithii gene expression in vivo in the presence of B. thetaiotaomicron vs. alone

TABLE 12
InterPro-based classification of adhesin-like proteins (ALPs)
in the M. smithii and M. stadtmanae proteomes

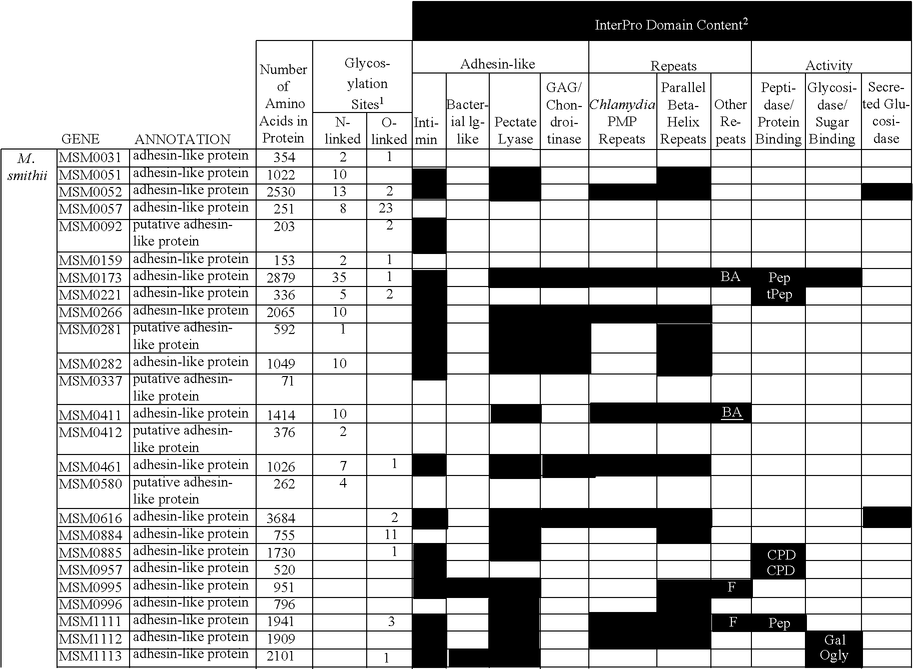

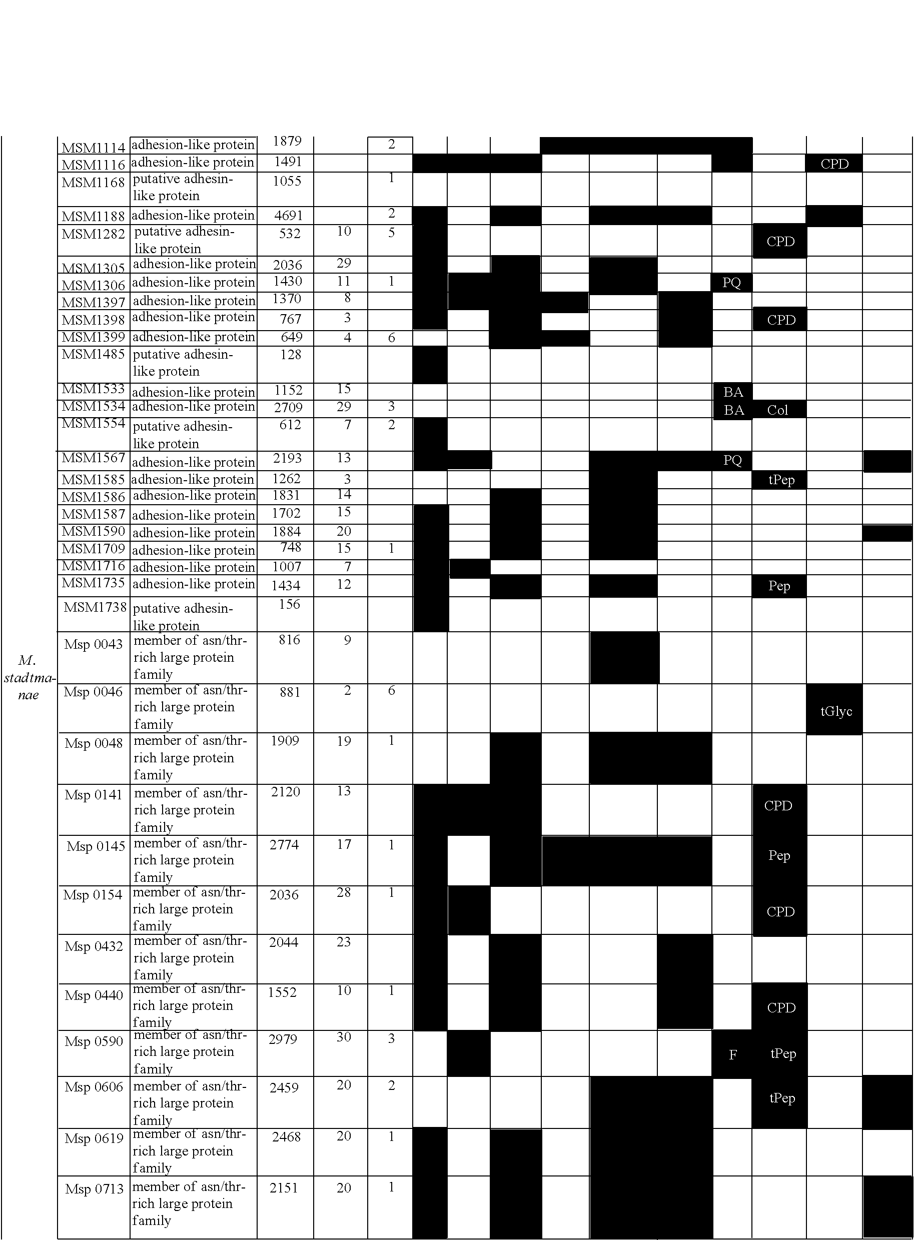

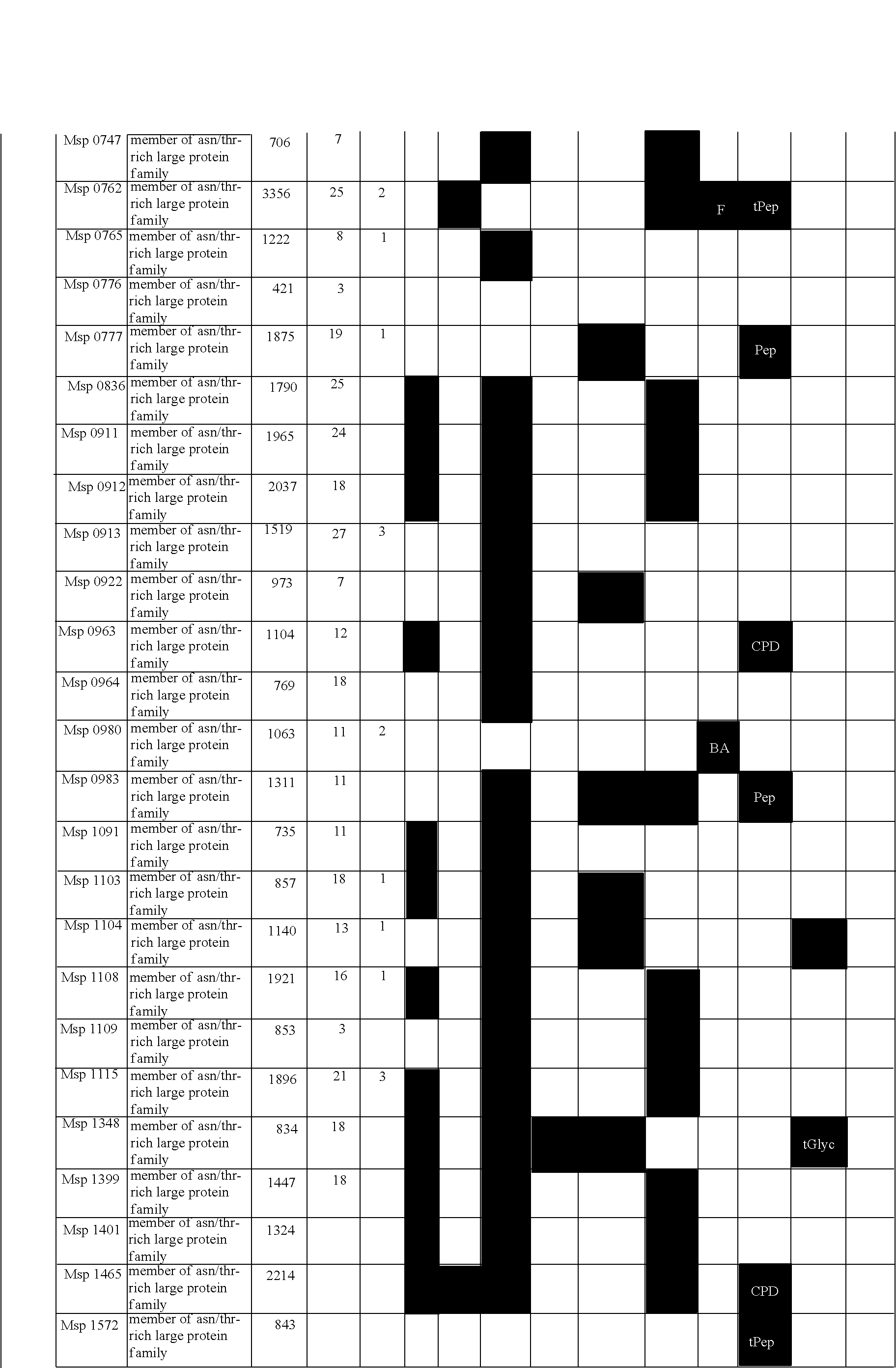

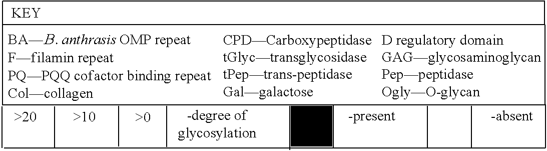

¹Predictions completed using NetNGlyc and NetOglyc (htt://www.cbs.dtu.dk/services/).
²InterPro domains: Invasin/intimin cell-adhesion (PR008964); Bacterial lg-like (IPR003344); pectin lyase fold (IPR011050); GAGlyase,Chondroitinase B-type (IPR12333); Polymorphic membrane protein, Chlamydia (IPR03368); Parallel beta-helix repeat (IPR006626); Peptidase S8 and S53 (IPR000209); Penicillin-binding protein, transpeptidase fold (IPR012338); Carboxypeptidase regulatory region (IPR008969)

TABLE 13
M. smithii GeneChip
Genes Probe Average number of Naming Prefix Represented
Probesets pairs probe pairs per probeset

| | | | | | |
|---|---|---|---|---|---|
| control sequences | AFFX | 64 | 64 | 1024 | 16 |
| protein coding genes | MSM | 1778 | 2018 | 19967 | 11 |
| tRNA genes (1-2 | MSM-tRNAxx | 34 | 74 | 450 | 11 |
| probesets/gene) | | | | | |
| rRNA genes¹ | MSMxx-rRNA | 8 | 7 | 77 | 11 |
| intergenic sequences | ig | | 1581 | 4931 | 3 |

¹Note that the M. smithii genome contains three 5S rRNA genes, one 7S rRNA gene, two 16S rRNA genes, and two 23S rRNA genes. Due to the high nucleotide sequence identity among rRNA genes of a given type, each is represented by a single probeset (the 16S rRNA probeset is replicated four times on the GeneChip

TABLE 14
BLAST analysis of the putative M. smithii prophage

| | Phage | | | | |
|---|---|---|---|---|---|
| M. smithii | Protein | | | | |
| Protein | Sequence ID* | Function | HMM Annotation | Phage HMM | E value |
| MSM1640 | 5417 | unknown | Phage_integrase: Phage integrase family | PF00589 | 2.30E−06 |
| MSM1654 | 5721 | Gp40 | ERF: ERF superfamily | PF04404 | 6.90E−11 |
| MSM1671 | 5397 | large terminase subunit | psiM2_ORF9: phage uncharacterized protein, | TIGR01630 | 0.0042 |
| | | | C-terminal domain | | |
| MSM1672 | 5398 | portal protein | portal_PBSX: phage portal protein, PBSX | TIGR01540 | 6.70E−12 |
| | | | family | | |
| MSM1675 | 6246 | putative structural protein | | | |
| MSM1677 | 6247 | putative structural protein | | | |
| MSM1684 | 20206 | ORF001 | TMP: TMP repeat | PF05017 | 0.0036 |
| MSM1691 | 6262 | PeiW | | | |

*from the Phage Sequence Databank

TABLE 15
Primers used for qRT-PCR assays
AMPLICON ORF ANNOTATION PRIMER SEQUENCE (5′ −> 3′) SIZE (bp)

| | | | | |
|---|---|---|---|---|
| MSM0027 | glutamate synthase, GltB | MSM0027.F | GAAGGCCGTCCGATAGGTA | 117 |
| | | MSM0027.R | CTCCAGTAGCTCCCCCTCTT | |
| MSM0049 | F420-dependent NADP reductase, Fno | MSM0049.F | GGGTTCAGCAGCAGAAAGG | 118 |
| | | MSM0049.R | CACATTCAATTGGGTCTGGA | |
| MSM0227 | HMG-CoA reductase, HmgA | MSM0227.F | GGCTGTGAATTACCGCATATGG | 117 |
| | | MSM0227.R | TAACGGTCCGGCTACACCTACA | |
| MSM0028 | succinyl-CoA synthetase, Suc | MSM0228.F | TGCTCGTGAAATGGACACTACAG | 165 |
| | | MSM0228.R | GTAAGCTGGCTGGCTACTTCGT | |
| MSM0234 | ammonium transporter, AmtB | MSM0234.F | TTTCTGGTGGTGTTGTTGGA | 115 |
| | | MSM0234.R | TAACCATCCTCCACCCCATA | |
| MSM0291 | bicarbonate ABC transporter, | MSM0291.F | TCTGCAGTACCGCCTATAGTTTCC | 101 |
| | permease component, BtcA | MSM0291.R | CCTAAACCGCTACTTGAACCTATCA | |
| MSM0330 | acetyl-CoA synthetase, Acs | MSM0330.F | ATCGAAGAGGAAAGCGATGA | 103 |
| | | MSM0330.R | GGAAGTCCGCTTGTACCTGA | |
| MSM0368 | glutamate synthase (NADPH), | MSM0368.F | GGAATGCTTCCTGAAGAACG | 127 |
| | alpha subunit, GltA | MSM0368.R | GCCCCCTGACCTATTTTGAT | |
| MSM0411 | adhesion-like protein | MSM0411.F | TCAGAATTGCAGGTGGTTTGG | 129 |
| | | MSM0411.R | CGTGAACATCCATCCCATTTAC | |
| MSM0515 | methanol: cobalamin methyl- | MSM0515.F | ATGTGGTGCAAAAGGACCTC | 112 |
| | transferase, MtaB | MSM0515.R | CAGAGTGTGCACAAACAGCA | |
| MSM0516 | corrinoid protein | MSM0516.F | CGTAGAAGCTTACCACACACCA | 108 |
| | | MSM0516.R | CGGTACGAATTCCCCTACAA | |
| MSM0518 | methylcobalamin: coenzyme M | MSM0518.F | TATTGCATATCTGCGGGTCA | 112 |
| | methyltransferase | MSM0518.R | GATGCTTTCCTTGGCTTTTG | |
| MSM0560 | pyruvate: ferredoxin | MSM0560.F | CAATCATTATCCGGAGCAATGG | 104 |
| | oxidoreductase, ProB | MSM0560.R | GGTGTTGCACCACTTCTTTGGA | |
| MSM0572 | methylene-H4MPT dehydrogenase, Hmd | MSM0572.F | ACCCAGGTGCTGTACCTGAAAT | 119 |
| | | MSM0572.R | TGTGAATGCAGATCCTCTTGCT | |
| MSM0654 | carbonic anhydrase, Cab | MSM0654.F | TGGTGCTGTTGTTCATGGAT | 112 |
| | | MSM0654.R | CAGCTCCAGCCCCTACAATA | |
| MSM0848 | ribofuranosylaminobenzene | MSM0848.F | CCAGCATTTGGCCATTCAA | 146 |
| | 5′-phosphate synthase, RfaS | MSM0848.R | GGTCCAAAAGAGCTCATACCTACAC | |
| MSM0888 | glutamate dehydrogenase, GhdA | MSM0888.F | TGCTCTTCCATGTGCAACTC | 100 |
| | | MSM0888.R | TAGGCATGTTTGCACCTTCA | |
| MSM0986 | conjugated bile salt acid | MSM0986.F | TTATAGTCGGGGAATGGGTTC | 109 |
| | hydrolase | MSM0986.R | TTTCAGAATCTCCGGAAACG | |
| MSM0988 | phosphoenolpyruvate synthase, PpsA | MSM0988.F | CAAGCTCATTATGGCGAACCA | 110 |
| | | MSM0988.R | GCTACGCCATTGTCATCACCTA | |
| MSM0991 | bicarbonate ABC transporter, | MSM0991.F | TTGCACGTGAAGACGGTTATG | 111 |
| | substrate-binding component, BtcB | MSM0991.R | CCTGACCCTGTTTAACTGCATCAT | |
| MSM0995 | adhesin-like protein | MSM0995.F | GTGATGCATTAGAAGAGGCTCCTT | 113 |
| | | MSM0995.R | ATCTCCCGCAGGCATGATAGTT | |
| MSM1014 | MtrE | MSM1014.F | AACAAAGCGGCTTCTGGTGAA | 127 |
| | | MSM1014.R | CGACACAAGATCCCATTGCAAT | |
| MSM1078 | sodium: bile transporter | MSM1078.F | GCTGTTTCTGGAAGTTCCGCTTA | 105 |
| | | MSM1078.R | CCTAGAAGCGGTGTCCAGATAAAGT | |
| MSM1112 | adhesion-like protein | MSM1112.F | GCTAAATTCACTGACAGCACAGGA | 114 |
| | | MSM1112.R | ACCCAAATCAGCTACACCGTCTT | |
| MSM1113 | adhesion-like protein | MSM1113.F | TCGCATAGGACTTGGATTAGGA | 107 |
| | | MSM1113.R | CAACAGCCCCTTCAATTAACCT | |
| MSM1198 | O-sialglycoprotein endopeptidase | MSM1198.F | GCTGCCGAACATCATGGAT | 162 |
| | | MSM1198.R | TAGTGCCAGTGTTCTTGCAGAA | |
| MSM1282 | adhesion-like protein | MSM1282.F | GCGGCATTATCTTTTTCAGCTG | 183 |
| | | MSM1282.R | AGCAGGTACATCCCCTCCAGTA | |
| MSM1305 | adhesion-like protein | MSM1305.F | ACATTAGACGGTCAAGGCAAACC | 131 |
| | | MSM1305.R | TATTCACCGGCCATCAGTCTGATT | |
| MSM1381 | alcohol dehydrogenase, Adh | MSM1381.F | AAGAAGTCCCGGAATGTGG | 102 |
| | | MSM1381.R | TCCGATAGCTCCTTCCCATA | |
| MSM1399 | adhesion-like protein | MSM1399.F | CTGCAACTACTTCTGGAGGATCA | 117 |
| | | MSM1399.R | CCATCACTAGAACCAGAGTCACTTG | |
| MSM1418 | glutamine synthetase, GlnA | MSM1418.F | GACGGAAAACCATTTGTTGG | 141 |
| | | MSM1418.R | GCATTGGGTATCCTTCATCG | |
| MSM1534 | adhesion-like protein | MSM1534.F | AATCCACATCTGATGCAGCTGTC | 239 |
| | | MSM1534.R | TCCCATGTCGGAGTTACAACA | |
| MSM1539 | sialic acid synthase, NeuB | MSM1539.F | TGGCAAAATCTGGTGCAGAT | 116 |
| | | MSM1539.R | CCTGACCGTCCCATATTGTTC | |

TABLE 16
M. smithii strain PS treated with varying concentrations of statins
Atorvastatin treated cells, average optical
density (600 nm), standard deviation

| 1 mM | 100 μM | 10 μM | methanol |
|---|---|---|---|
| 0.032 (0.006) | 0.032 (0.003) | 0.03 (0.001) | 0.032 (0.004) |
| 0.018 (0.002) | 0.032 (0.007) | 0.042 (0.004) | 0.07 (0.007) |
| 0.001 (0.003) | 0.031 (0.006) | 0.09 (0.005) | 0.135 (0.008) |
| 0.001 (0.004) | 0.03 (0.007) | 0.079 (0.027) | 0.13 (0.012) |
| 0.008 (0.004) | 0.033 (0.007) | 0.139 (0.043) | 0.234 (0.018) |
| 0.007 (0.012) | 0.033 (0.002) | 0.233 (0.11) | 0.195 (0.05) |
| 0.001 (0.006) | 0.024 (0.007) | 0.115 (0.045) | 0.218 (0.064) |

Pravastatin treated cells, average optical
density (600 nm), standard deviation

| 1 mM | 100 μM | 10 μM | ethanol |
|---|---|---|---|
| 0.034 (0.003) | 0.035 (0.003) | 0.039 (0.004) | 0.036 (0.005) |
| 0.036 (0.006) | 0.069 (0.02) | 0.066 (0.003) | 0.072 (0.012) |
| 0.031 (0.003) | 0.104 (0.03) | 0.097 (0.025) | 0.128 (0.011) |
| 0.038 (0.003) | 0.104 (0.024) | 0.084 (0.009) | 0.109 (0.011) |
| 0.026 (0.006) | 0.139 (0.078) | 0.08 (0.014) | 0.223 (0.015) |
| 0.016 (0.01) | 0.217 (0.175) | 0.181 (0.048) | 0.258 (0.105) |
| 0.017 (0.004) | 0.297 (0.111) | 0.039 (0.015) | 0.212 (0.113) |

Rosuvastatin treated cells, average optical
density (600 nm), standard deviation

| 1 mM | 100 μM | 10 μM | DMSO |
|---|---|---|---|
| 0.031 (0.002) | 0.031 (0.003) | 0.034 (0.002) | 0.033 (0.002) |
| 0.024 (0.006) | 0.026 (0.009) | 0.068 (0.006) | 0.075 (0.006) |
| 0.017 (0.006) | 0.021 (0.002) | 0.101 (0.009) | 0.125 (0.013) |
| 0.03 (0.014) | 0.02 (0.004) | 0.082 (0.011) | 0.093 (0.007) |
| 0.013 (0.008) | 0.027 (0.016) | 0.122 (0.039) | 0.152 (0.05) |
| 0.018 (0.004) | 0.033 (0.005) | 0.159 (0.058) | 0.117 (0.029) |
| 0.003 (0.002) | 0.033 (0.042) | 0.174 (0.146) | 0.183 (0.071) |

TABLE 17
M. smithii strain F1 treated with varying concentrations of statins
Atorvastatin treated cells, average optical
density (600 nm), standard deviation

| 1 mM | 100 μM | 10 μM | methanol |
|---|---|---|---|
| 0.015 (0.006) | 0.01 (0) | 0.019 (0.001) | 0.015 (0.003) |
| 0.008 (0.014) | 0.018 (0.001) | 0.039 (0.004) | 0.045 (0.003) |
| 0.013 (0.01) | 0.018 (0.002) | 0.039 (0.007) | 0.069 (0.002) |
| 0.004 (0.014) | 0.018 (0.003) | 0.056 (0.011) | 0.092 (0.003) |
| 0.001 (0.011) | 0.016 (0.002) | 0.061 (0.023) | 0.115 (0.008) |
| 0.001 (0.015) | 0.015 (0.001) | 0.084 (0.033) | 0.155 (0.019) |

Pravastatin treated cells, average optical
density (600 nm), standard deviation

| 1 mM | 100 μM | 10 μM | ethanol |
|---|---|---|---|
| 0.011 (0.001) | 0.007 (0.002) | 0.019 (0.001) | 0.017 (0.002) |
| 0.022 (0.002) | 0.047 (0.004) | 0.05 (0.003) | 0.05 (0.005) |
| 0.026 (0.003) | 0.066 (0.004) | 0.071 (0.003) | 0.073 (0.006) |
| 0.026 (0.003) | 0.085 (0.008) | 0.102 (0.003) | 0.095 (0.004) |
| 0.022 (0.002) | 0.089 (0.01) | 0.124 (0.004) | 0.121 (0.011) |
| 0.018 (0.003) | 0.133 (0.029) | 0.168 (0.004) | 0.153 (0.024) |

Rosuvastatin treated cells, average optical
density (600 nm), standard deviation

| 1 mM | 100 μM | 10 μM | DMSO |
|---|---|---|---|
| 0.015 (0.003) | 0.01 (0.003) | 0.021 (0.003) | 0.015 (0.003) |
| 0.016 (0.003) | 0.026 (0.003) | 0.046 (0.004) | 0.043 (0.001) |
| 0.019 (0.003) | 0.027 (0.004) | 0.057 (0.002) | 0.062 (0.003) |
| 0.019 (0.003) | 0.026 (0.004) | 0.081 (0.008) | 0.081 (0.005) |
| 0.018 (0.003) | 0.025 (0.001) | 0.085 (0.021) | 0.103 (0.005) |
| 0.02 (0.006) | 0.016 (0.003) | 0.094 (0.048) | 0.102 (0.017) |

TABLE 18
M. smithii strain ALI treated with varying concentrations of statins
Atorvastatin treated cells, average optical
density (600 nm), standard deviation

| 1 mM | 100 μM | 10 μM | methanol |
|---|---|---|---|
| 0.01 (0.008) | 0.015 (0.003) | 0.012 (0.002) | 0.019 (0.004) |
| 0.016 (0.007) | 0.008 (0.002) | 0.026 (0.016) | 0.043 (0.015) |
| 0.052 (0.063) | 0.002 (0.001) | 0.058 (0.084) | 0.046 (0.022) |
| 0.018 (0.028) | 0.014 (0.016) | 0.072 (0.066) | 0.074 (0.024) |
| 0.025 (0.043) | 0.008 (0.014) | 0.031 (0.046) | 0.06 (0.044) |
| 0.01 (0.012) | 0.001 (0) | 0.024 (0.02) | 0.093 (0.053) |

Pravastatin treated cells, average optical
density (600 nm), standard deviation

| 1 mM | 100 μM | 10 μM | ethanol |
|---|---|---|---|
| 0.013 (0.002) | 0.011 (0.003) | 0.015 (0.001) | 0.025 (0.009) |
| 0.036 (0.045) | 0.054 (0.036) | 0.06 (0.027) | 0.047 (0.012) |
| 0.103 (0.176) | 0.072 (0.076) | 0.071 (0.037) | 0.061 (0.026) |
| 0.051 (0.027) | 0.079 (0.122) | 0.086 (0.048) | 0.083 (0.036) |
| 0.018 (0.026) | 0.104 (0.154) | 0.083 (0.053) | 0.083 (0.038) |
| 0.081 (0.032) | 0.091 (0.143) | 0.116 (0.05) | 0.111 (0.047) |

Rosuvastatin treated cells, average optical
density (600 nm), standard deviation

| 1 mM | 100 μM | 10 μM | DMSO |
|---|---|---|---|
| 0.017 (0.007) | 0.029 (0.016) | 0.019 (0.005) | 0.014 (0.002) |
| 0.032 (0.02) | 0.033 (0.037) | 0.044 (0.008) | 0.04 (0.007) |
| 0.02 (0.02) | 0.012 (0.009) | 0.038 (0.011) | 0.044 (0.008) |
| 0.013 (0.01) | 0.028 (0.021) | 0.056 (0.036) | 0.058 (0.006) |
| 0.015 (0.009) | 0.015 (0.018) | 0.074 (0.036) | 0.085 (0.003) |
| 0.016 (0.01) | 0.015 (0.026) | 0.1 (0.02) | 0.126 (0.013) |

TABLE 19
M. smithii strain B181 treated with
varying concentrations of statins
Atorvastatin treated cells, average optical
density (600 nm), standard deviation

| 1 mM | 100 μM | 10 μM | methanol |
|---|---|---|---|
| 0.007 (0.004) | 0.004 (0.001) | 0.011 (0.001) | 0.007 (0.003) |
| 0.018 (0.001) | 0.013 (0.003) | 0.032 (0.007) | 0.034 (0.006) |
| 0.014 (0.003) | 0.005 (0.002) | 0.032 (0.006) | 0.046 (0.022) |
| 0.009 (0.002) | 0.003 (0.005) | 0.04 (0.008) | 0.07 (0.029) |
| 0.01 (0.004) | 0.003 (0) | 0.044 (0.011) | 0.121 (0.027) |
| 0.01 (0.003) | 0.006 (0.001) | 0.048 (0.009) | 0.133 (0.026) |

Pravastatin treated cells, average optical
density (600 nm), standard deviation

| 1 mM | 100 μM | 10 μM | ethanol |
|---|---|---|---|
| 0.007 (0.001) | 0.003 (0.001) | 0.011 (0.001) | 0.009 (0.003) |
| 0.019 (0.003) | 0.039 (0.005) | 0.047 (0.002) | 0.039 (0.013) |
| 0.015 (0.008) | 0.061 (0.004) | 0.061 (0.003) | 0.048 (0.03) |
| 0.014 (0.001) | 0.088 (0.002) | 0.102 (0.007) | 0.094 (0.075) |
| 0.016 (0.002) | 0.114 (0.006) | 0.135 (0.01) | 0.137 (0.057) |
| 0.015 (0.006) | 0.171 (0.031) | 0.198 (0.02) | 0.14 (0.037) |

Rosuvastatin treated cells, average optical
density (600 nm), standard deviation

| 1 mM | 100 μM | 10 μM | DMSO |
|---|---|---|---|
| 0.01 (0) | 0.006 (0.006) | 0.012 (0.002) | 0.005 (0.004) |
| 0.016 (0.004) | 0.013 (0.003) | 0.029 (0.001) | 0.032 (0.003) |
| 0.011 (0.002) | 0.008 (0.001) | 0.04 (0.001) | 0.066 (0.02) |
| 0.01 (0.005) | 0.007 (0.003) | 0.066 (0.005) | 0.095 (0.018) |
| 0.014 (0.004) | 0.004 (0.002) | 0.097 (0.014) | 0.148 (0.032) |
| 0.008 (0.003) | 0.001 (0.002) | 0.121 (0.012) | 0.194 (0.073) |

### Materials and Methods for Examples 6-11

Isolation and Culturing of M. smithii from Human Fecal Samples

Two gallon stainless steel paint canisters (Binks; catalog number 83S-210) were modified for incubation of plates at 37° C. in an oxygenfree mixture of 20% CO₂/80% H₂ at a pressure of 15 psi. Canisters contained a heating element (Electro-Flex Pail Heaters) regulated by a custom designed controller consisting of a 16A2120 temperature/process control (Love Controls; Dwyer Instruments), a resistance temperature detector probe to measure the internal tank temperature, and several safety features to prevent overheating or burns. Pressure in each tank was measured and recorded with a digital manometer (LEO record; Omni Instruments). The apparatus was housed inside an anaerobic chamber (COY Labs). All human fecal samples used in this study were obtained by using protocols approved by the Washington University Human Research Protection Office and its constituent review committees. All samples were deidentified and assigned codes as described in a previous publication (65): Information about the age and BMI of the donors can also be found in this publication. All samples were frozen at −20° C. within 30 min after they had been produced by donors; they were then placed in a standard −80° C. freezer no more than 24 h later and stored at this temperature for at least 1 yr prior to their use in the present study. An ≈2-g aliquot of a given frozen fecal sample was thawed (inside of the Coy anaerobic Chamber) and serially diluted in modified MBC medium (66) within the anaerobic chamber. Aliquots of serial dilutions (10⁻² to 10⁻⁸) were transferred to 14 mL of MBC supplemented with 5% rumen fluid, 10 μg/mL erythromycin, 1 μg/mL ampicillin, 10 μg/mL vancomycin and 10 mg/mL amphotericin B. The mixture was introduced into 125-mL serum bottles (Bellco Glass). These enrichment cultures were incubated under a fully deoxygenated atmosphere of 20% CO₂/80% H₂ (30 psi of pressure) at 37° C. After at least 7 d, aliquots were plated onto MBC noble agar and the plates were incubated in the custom pressurized tanks described above for colony isolation. In parallel, the same serial dilutions were spread directly onto MBC noble agar plates with antibiotics. All plates were incubated under an atmosphere of 20% CO₂/80% H₂ (15 psi of pressure) in our custom PHAT (Pressurized Heated Anaerobic Tank) system at 37° C. Colonies were picked and screened by PCR of their 16S rRNA genes by using bacterial primers 8F (5′-AGAGTTTGATCCTGGCTCAG-3′) and 1391R (5′-GACGGGCGGTGWGTRCA-3′) and archaeal primers 571aF (5′-GCYTAAAGSRICCGTAGC-3′) and 958aR (5′-YCCGGCGTTGAMTCCAATT-3′). Amplicons generated from archaeal-directed primers were sequenced using the method of Sanger (Retrogen).

Pure isolates were then cultured anaerobically in MBC medium in a fully deoxygenated atmosphere of 20% CO₂/80% H₂ (30 psi of pressure) at 37° C. Cells were harvested by centrifugation, and DNA was isolated by phenol-chloroform and ethanol precipitation, as described (50). The purity of each DNA preparation was verified by gel electrophoresis.

qPCR Assay of mcrA in Human Fecal Samples

Frozen fecal samples were pulverized by manual grinding under liquid nitrogen, and crude DNA was isolated by bead beating and phenol/chloroform extraction. The Qiagen Blood and Tissue kit was used to clean up the crude DNA and remove RNA and protein. Twenty nanograms of purified community DNA was amplified by using an Mx3000 real-time PCR system (Stratagene) in 25-μL reaction mixtures containing SYBR-green and 0.8 μM McrA_MLf/r primers (5′-GGTGGTGTMGGATTCACACARTAYGCWACAGC-3′ and 5′-TTCATTGCRTAGTTWGGRTAGTT-3′; ref. 14), which amplified a ≈450-bp region of mcrA. Cycling conditions were as follows: 40 cycles of denaturing at 94° C. for 45 s, annealing 56° C. for 45 s, extension 72° C. for 30 s, with collections at 79-81° C. A subsequent dissociation curve was used to examine the homogeneity of amplicons, to detect the presence of primer dimers, and to determine the appropriate collection temperature.

A standard curve was constructed with purified M. smithii gDNA at concentrations ranging from 0.01 ng to 10 ng and used to define the concentration of mcrA DNA in each of the fecal DNA samples. Based on the known genome size of M. smithii PS, we expressed the data as number of genome equivalents (GE) per ng of total fecal DNA. Samples that only produced detectable amplification after 37 cycles of PCR were scored as “negative,” as were samples having <40 GE per ng of DNA. Data were not normally distributed; therefore, a log-base 10 transformation was performed.

A subset of samples was selected for amplicon sequencing to determine the identity and diversity of mcrA sequences amplified by these primers, and whether archaeal DNA was present in these samples that was not found by our mcrA-based primers. The latter was determined by using PCR primers directed at archaeal 16S rRNA genes [571aF (5′-GCYTAAAGSRICCGTAGC-3′; ref. 63) and 958aR (5′-YCCGGCGTTGAMTCCAATT-3′; ref. 64)] and the following cycling conditions; 30 cycles of denaturing at 94° C. for 2 min, annealing at 65° C. for 45 s, and extension at 72° C. for 2 min. Amplicons were sequenced using the method of Sanger (Retrogen).

### Genome Sequencing

Methanobrevibacter smithii strain PS (ATCC 35061) was grown as described above for 6d at 37° C. DNA was recovered from harvested cell pellets using the QIAGEN Genomic DNA Isolation kit with mutanolysin (1 unit/mg wet weight cell pellet; Sigma) added to facilitate lysis of the microbe. An ABI 3730xl instrument was used for paired end-sequencing of inserts in a plasmid library (average insert size 5 Kb; 42,823 reads; 11.6×-fold coverage), and a fosmid library (average insert size of 40 Kb; 7,913 reads; 0.6×-fold coverage). Phrap and PCAP (Huang et al. (2003) Genome Res 13:2164-70) were used to assemble the reads. A primer-walking approach was used to fill-in sequence gaps. Physical gaps and regions of poor quality (as defined by Consed; Gordon et al., (1998) Genome Res. 8, 195-202) were resolved by PCR-based re-sequencing. The assembly's integrity and accuracy was verified by clone constraints. Regions containing insufficient coverage or ambiguous assemblies were resolved by sequencing spanning fosmids. Sequence inversions were identified based on inconsistency of constraints for a fraction of read pairs in those regions. The final assembly consisted of 12.6× sequence coverage with a Phred base quality value ≧40. Open-reading frames (ORFs) were identified and annotated as described below.

### Horizontal Gene Transfer (HGT) Analysis

For each gene call, compositional statistics were calculated by using the PyCogent code base (67). The statistics included the GC content at each position, three versions of the dinucleotide use (overlapping, nonoverlapping, or “3-1”), all K-words ranging from length 1 through 6, and codon use (Table 20 and 21). For each M. smithii strain, the composition of each gene was compared against (i) the composition of the genome as a whole and (ii) the composition of highly expressed genes. Genes that mapped to the KEGG orthology (KO) groups for ribosomal proteins were used to calculate the highly expressed test set. The gene and control vectors were compared using either the G-test statistic or Pearson correlation.

TABLE 20
Compositional evidence for HGT in adhesin-like proteins

| | | | | Fold |
|---|---|---|---|---|
| | | Atypical/ | Per- | enrich- |
| Method | Significance Measure | total | cent | ment* |
| 3-1 Dinucleotide | Rank order threshold; | 558/853 | 65% | 6.4 |
| | G-score | | | |
| Codon Usage | Rank order threshold; | 525/853 | 63% | 6.6 |
| | G-score | | | |
| K-words (length 4) | Rank order threshold; | 538/853 | 62% | 8.1 |
| | G-score | | | |
| K-words (length 6) | Rank order threshold; | 445/853 | 52% | 9.3 |
| | G-score | | | |

*Fold-enrichment is relative to the overall levels of HGT predicted by a given method.

TABLE 21
Compositional evidence for HGT in the M. smithii genome

| Method | Significance Measure | Atypical/Total | Percent |
|---|---|---|---|
| 3-1 Dinucleotide | Rank order threshold; | 4200/41694 | 10.1% |
| | G-score | | |
| 3-1 Dinucleotide | Rank order threshold; | 1410/41694 | 3.3% |
| | Pearson correlation | | |
| Codon Usage | Rank order threshold; | 3973/41694 | 9.5% |
| | G-score | | |
| Codon Usage | Rank order threshold; | 1675/41694 | 4.0% |
| | Pearson correlation | | |
| K-words (length 4) | Rank order threshold; | 3230/41694 | 7.7% |
| | G-score | | |
| K-words (length 4) | Rank order threshold; | 223/41694 | 5.3% |
| | Pearson correlation | | |
| K-words (length 6) | Rank order threshold; | 2336/41694 | 5.6% |
| | G-score | | |
| K-words (length 6) | Rank order threshold; | 3300/41694 | 7.9% |
| | Pearson correlation | | |

The significance of the results was calculated in two ways; first, the Bonferroni corrected P value was calculated for the G-test; second, because the distribution of compositional counts may violate normality, the method of picking significance thresholds based on the rank order of gene scores of Tsirigos et al. (57) was employed.

Because highly expressed genes frequently possess unusual gene compositions, gene transfer was predicted only in cases where the gene did not match the whole-genome model, and the gene also did not match the highly expressed model. Annotated tRNAs and rRNAs were also excluded from the analysis.

Phylogenetic confirmation of gene transfers predicted by compositional means was performed using the RIATA-HGT program of PhyloNet version 1.7 (68). We obtained all available gene sequences for all KO groups that contained one or more M. smithii genes. Annotations for gene family level KEGG assignments were obtained by blasting each protein sequence against version 54 of the KEGG database. The best hit with a KEGG assignment was taken. Multiple assignments were given if the best hit had more than one annotation.

Python scripts were used to generate separate FASTA files for each orthology group containing the amino acid sequences for M. smithii and KEGG proteins. All sequences for each orthology group were then separately aligned in MUSCLE (69) by using maxiters=4, and gene trees for each group were constructed in FASTTREE (70).

PhyloNet requires that no paralogs be present on protein trees. Therefore, multiple members of a KO present in a single KEGG genome were reduced to a single copy by removing sequences that produced the longest branches on the resulting phylogenetic tree. However, for M. smithii genes, we wanted to ensure that the process of paralog resolution did not prevent detection of possible xenologs (extra gene copies introduced by gene transfer). Therefore, all M. smithii genes were retained in each gene tree in the analysis. The species tree used consisted of the KEGG 16S rRNA sequences for each lineage in the tree, gathered by BLAST against the E. coli rrsG gene, and alignment in PyNAST. The location of “msi,” the M. smithii strain present in KEGG, was taken as the tree position for all M. smithii.

Because all multiple copies of gene family members were retained in M. smithii genomes, it was necessary to introduce an artificial polytomy into the species tree at the location of msi, with one tip for each paralog/strain combination. This approach is identical to separately running each gene copy, but is computationally more tractable because it avoids reinferring all transfers not involving M. smithii across the rest of the tree many times.

### Microbial RNA-Seq.

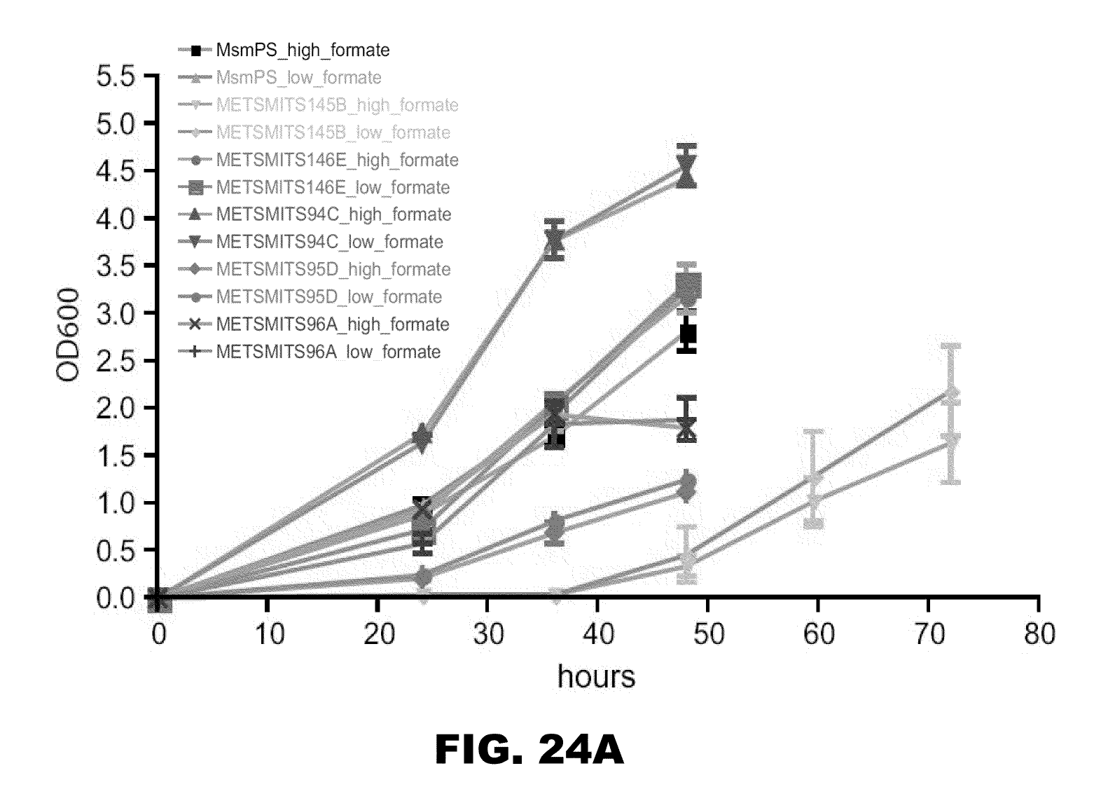

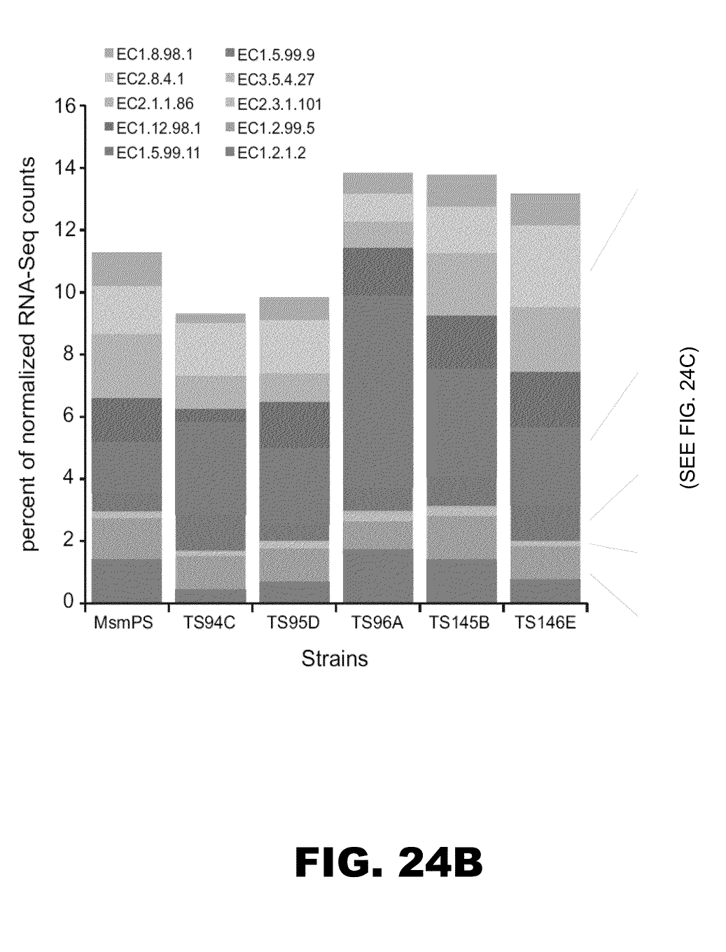

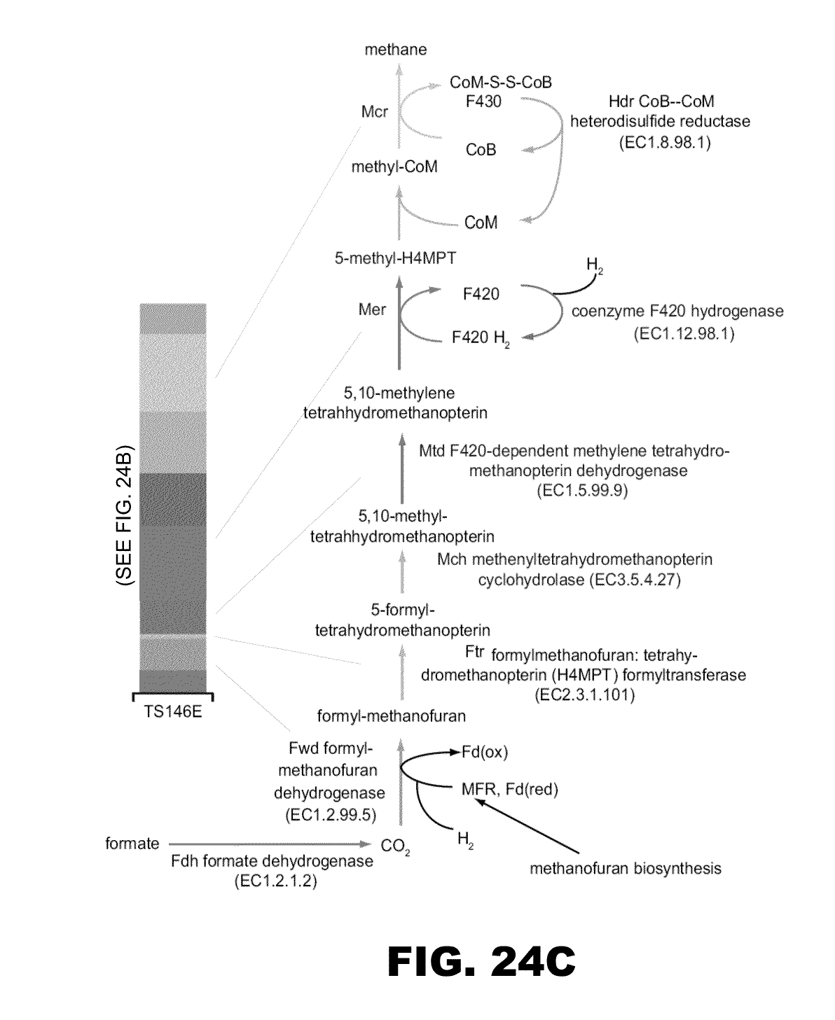

M. smithii strains were grown in standard MBC medium containing 2.8 or 44.1 mM formate. Medium was prepared anaerobically and aliquoted into 125-mL serum bottles, which were sealed and autoclaved. Triplicate cultures of each strain and condition were grown at 37° C. with agitation (100 rpm), in serum bottles containing 21 mL of medium plus 0.5 mL of 2.5% Na₂S, under an atmosphere of 80% H₂ and 20% CO₂ that was replenished every 6 h to a pressure of 30 psi. Seven milliliters of the culture were harvested at 36 h (FIG. 24A), placed directly into an equal volume of RNA-Protect (Qiagen), incubated for 5 min at room temperature, then centrifuged for 15 min at 3,220×g at 4° C. RNA was harvested by bead beating and phenol-chloroform extraction, and then treated with Turbo DNase (Ambion) and Baseline-ZERO DNase (Epicenter) to remove genomic DNA (71). RNA was then purified with the MEGAClear kit (Ambion), which also removes tRNAs and 5S rRNA. Ribosomal RNA was further depleted by using custom biotinylated oligos (Table 22) bound to magnetic Streptavidin Dynabeads (Invitrogen). Depleted RNA was reverse transcribed to doublestranded cDNA, then prepared for sequencing on an Illumina GAIIx instrument with 4 nucleotide barcoded adapters (71). Reads were assigned to barcodes, rRNA sequences were pruned, and the remaining reads were mapped to each strain's genome by using custom scripts (71) that use the ssaha mapping algorithm (72).

TABLE 22
Sequences of depletion oligos designed to remove
M. smithii 16S and 23s rRNAs.

| Name | Sequence |
|---|---|
| 16S_depl_61 | CTACGACTAAGTTTAGAGGATTACCTCCGC |
| 16S_depl_346 | TTGTCTCAGGTTCCATCTCCGGGCTCTTGC |
| 16S_depl_595 | CTAAGGGTAGGTTATCCACGTGTTACTGAG |
| 16S_depl_746 | AGGACTACCCGGGTATCTAATCCGGTTCGC |
| 16S_depl_1092 | GCGTGGGTCTCGCTCGTTGCCTGACTTAAC |
| 16S_depl_269 | AAAAGGGATTCAGTTTGTTCTAAGTCGATT |
| 16S_depl_733 | TTCCCTACGACTACAAGGATAAAAACCTTT |
| 16S_depl_1146 | AGTCTGAGTTGGTTTCTCTTTCGGGACACA |
| 16S_depl_1401 | CTGCTACTACTACCAGGATCCACATACCTG |
| 16S_depl_2644 | CAGGATGGAAAGAACCGACATCGAAGTAGC |
| 16S_depl_2704 | CCAGCTCACGTTCCCCTTTAATGGGCGAAC |

Comparison of RNA-Seq and Custom Affymetrix M. smithii GeneChips

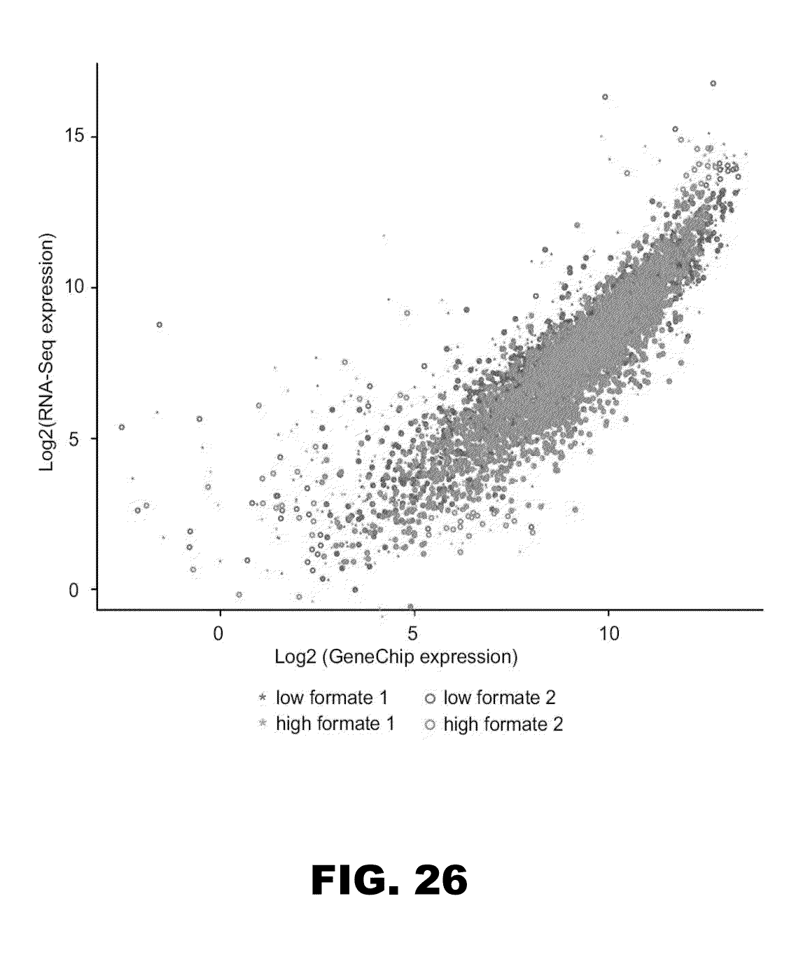

RNA from four samples of M. smithii PS (two replicates at each formate concentration) were split into aliquots for subsequent GeneChip target preparation, or for rRNA depletion and RNASeq. Nearly 106 million 36-nt Illumina GA-IIx reads were generated from the 4 samples (each sample run on a single lane of the eight-lane flow cell): 7.2 million of these reads mapped to coding regions (6.9%), whereas the remaining reads mapped to rRNA genes or other noncoding regions of the genome. Tables 20-31 were also generated for each replicate sample by using custom M. smithii GeneChips that have been described in an earlier report (50). GeneChip data were processed (see ref. 50 for details), and the resulting datasets were compared with RNA-Seq data (counts per million reads, normalized for gene length). The results obtained with each type of data were highly similar: Pearson's correlation r² values ranged from 0.86 to 0.89 for each replicate (P<2e⁻¹⁶; FIG. 26).

### Other Methods

Analyses of familial concordance or correlation for methanogen carriage or levels, and of their associations with overweight/obesity, were conducted by using logistic or linear regression, a robust variance estimator to adjust for the nonindependence of observations on family members.

### Example 6

### Detection of Insertion Sequence (IS) Elements and Prophages

A putative rearrangement was discovered in the M. smithii PS type strain by aligning draft assemblies of other strains using Mauve (49). This putative rearrangement is further evidenced by flanking transposases (Msm1419, Msm0730). When the type strain was first sequenced (50), a large number of genes predicted to be involved in genome evolution was noted: restriction modification systems, transposases, recombinases, and insertion sequence (IS) elements. IS finder (www-is.biotoul.fr) was able to detect matches to a known M. smithii IS element, ISM1, which is a member of the ISNCY family, and no other significant matches. However, the number of matches varied between strains quite considerably (Table 23).

A recent metagenomic study of the fecal viromes of adult female MZ twins showed that viromes are unique to individuals regardless of their degree of genetic relatedness. Intrapersonal diversity is very low with >95% of virotypes retained over a 1-yr period. Moreover, an individual's virome is dominated by a few temperate phage that exhibit remarkable genetic stability. These results indicated that a predatory Lotka-Volterra (LV)/Kill-the-Winner dynamic manifest in a number of other characterized environmental ecosystems is notably absent in the distal intestine where a more temperate phage lifestyle is evident (51). Therefore, it was of interest to characterize phage diversity in M. smithii as a function of host and family.

Prophages were detected by PhageFinder (52) in 7 of the 20 strains, including 4 of the 5 strains isolating one of the dizygotic twins (TS146), one strain from her co-twin (TS145), and two strains from their mother (TS147). When prophage sequences were blasted against the other strains, prophages were identified in two more strains, one from the mother of the MZ twins (METSMITS96C), and another from TS145 (METSMITS145A) (Table 23).

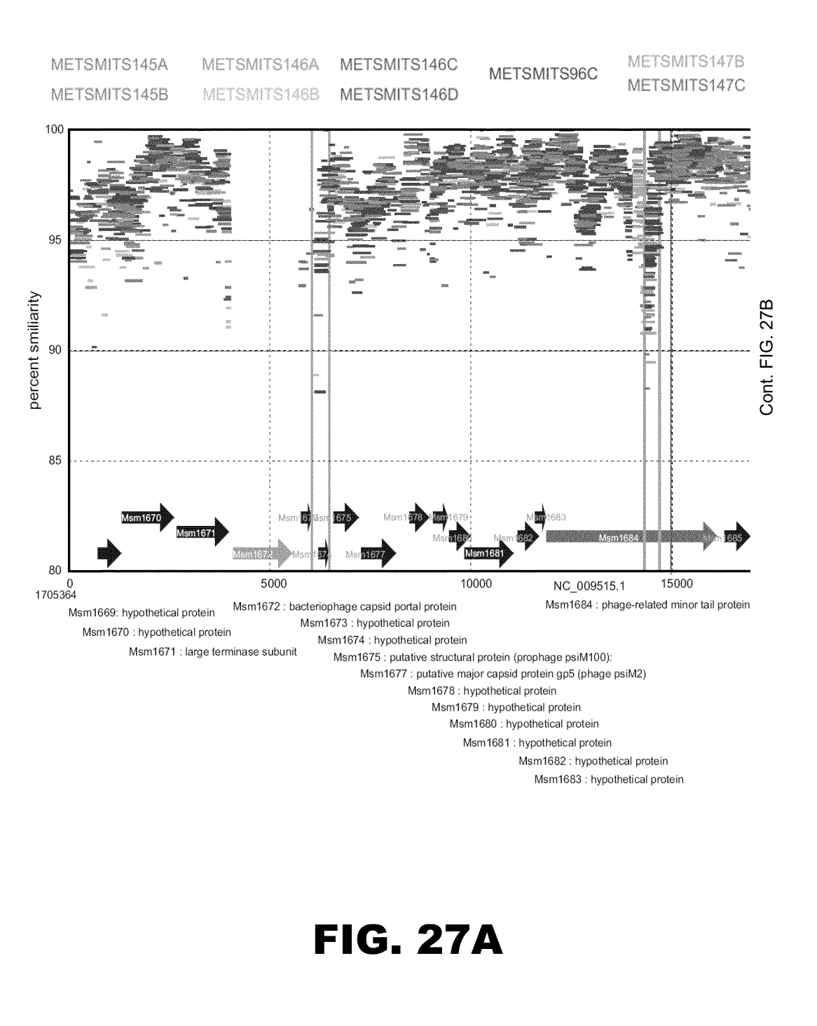

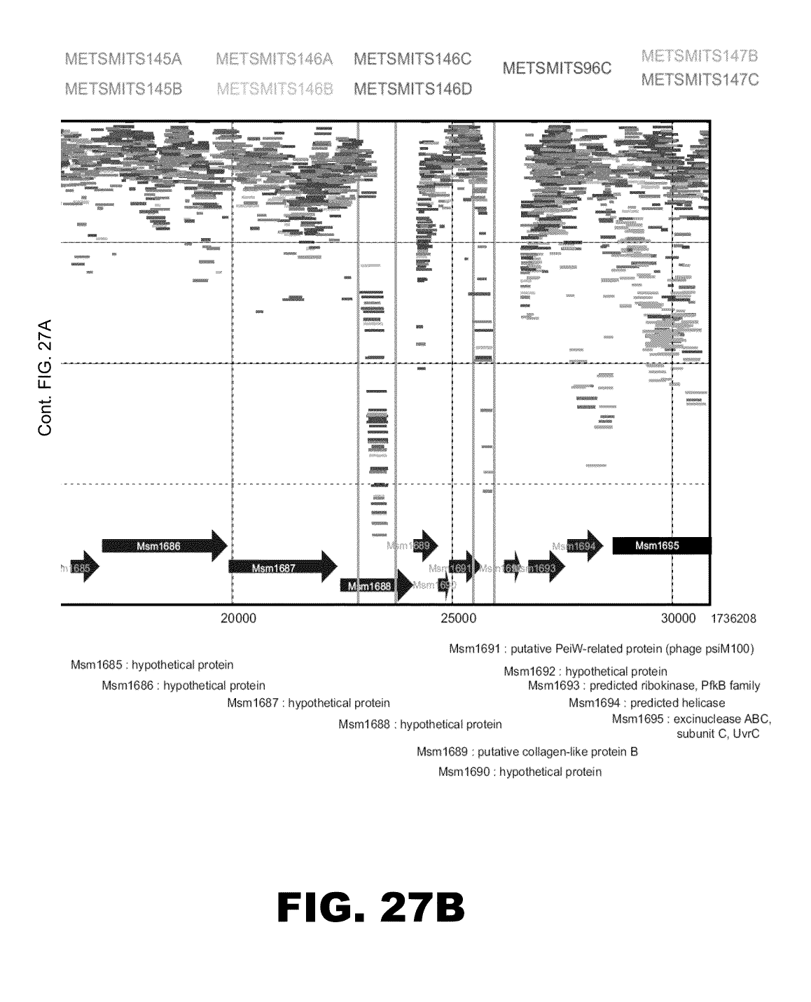

To identify regions of variation within these prophage, raw 454 Titanium reads for each strain were aligned (nucmer; ref. 53) to the prophage sequence of the PS type strain (coordinates 1705364:1736208). The results were plotted with Mummer (53) and overlayed to create a single plot with the PS type strain prophage gene calls displayed (FIG. 27). Regions of greatest variation in the prophage were in genes encoding the phage's tail protein (Msm1684), a putative PeiW-related protein (Msm1691, a predicted pseudomurein endoisopeptidase; see ref. 54) and several hypothetical proteins (Msm1674 and Msm1688).

TABLE 23
Summary of genome sequencing effort, assembly statistics and annotation results obtained for the
20 strains isolated in the present study ( Examples 6-11) and 3 previously identified isolates.

| | | number of | number of | | N50 | total |
|---|---|---|---|---|---|---|
| | | 36 nt | 454 Titanium | number of | contig | assembly |
| | strain name | Illumina reads | reads | contigs | size | size |
| MZ twin 1 | METSMITS94A | 5,049,552 | 449,545 | 47 | 120,002 | 1,889,378 |
| | METSMITS94B | 4,785,200 | 76,513 | 58 | 90,573 | 1,886,020 |
| | METSMITS94C | 20,939,658 | 433,652 | 50 | 108,845 | 1,910,054 |
| MZ twin 2 | METSMITS95A | 6,264,402 | 73,255 | 56 | 77,936 | 1,992,157 |
| | METSMITS95B | 3,557,512 | 85,737 | 44 | 133,694 | 1,972,498 |
| | METSMITS95C | 4,559,830 | 96,757 | 37 | 96,923 | 1,978,848 |
| | METSMITS95D | 22,316,058 | 415,598 | 58 | 94,662 | 2,011,683 |
| Mother of | METSMITS96A | 29,499,134 | 260,162 | 47 | 98,370 | 1,975,004 |
| MZ twins | METSMITS96B | 28,356,554 | 274,657 | 45 | 94,662 | 1,869,210 |
| | METSMITS96C | 25,292,727 | 190,329 | 108 | 43,698 | 1,818,239 |
| DZ twin 1 | METSMITS145A | 6,536,457 | 83,667 | 44 | 103,481 | 1,782,572 |
| | METSMITS145B | 8,277,390 | 45,203 | 54 | 80,226 | 1,797,373 |
| DZ twin 2 | METSMITS146A | 27,011,849 | 49,854 | 66 | 73,601 | 1,791,997 |
| | METSMITS146B | 26,899,427 | 58,633 | 43 | 147,680 | 1,794,702 |
| | METSMITS146C | 8,007,300 | 27,844 | 102 | 43,081 | 1,947,483 |
| | METSMITS146D | 9,210,075 | 73,182 | 33 | 139,646 | 1,713,264 |
| | METSMITS146E | 9,763,978 | 107,106 | 64 | 81,915 | 1,952,171 |
| Mother of | METSMITS147A | 10,284,342 | 375,219 | 61 | 87,700 | 2,008,979 |
| DZ twins | METSMITS147B | 8,551,491 | 230,907 | 40 | 99,611 | 1,965,064 |
| | METSMITS147C | 9,321,088 | 68,487 | 40 | 256,349 | 1,973,030 |
| Culture | MsmPS | | | 1 | | 1,853,160 |
| Collection | (NC_009515) | | | | | |
| (previously | METSMIALI | | | 24 | 226,159 | 1,704,865 |
| sequenced) | (DSM2375) | | | | | |
| | METSMIF1 | | | 25 | 1,043,555 | 1,727,775 |
| | (DSM2374) | | | | | |

| | | | | | number of | |
|---|---|---|---|---|---|---|
| | | | | | IS elements | presence |
| | coverage by | coverage by | total fold- | number | identified total | of |
| strain name | Illumina | Titanium | coverage | of CDS | (number >58 nt) | prophage |
| METSMITS94A | 96 | 83 | 179 | 1808 | 12(7) | |
| METSMITS94B | 91 | 14 | 106 | 1856 | 12(9) | |
| METSMITS94C | 395 | 79 | 474 | 1812 | 13(9) | |
| METSMITS95A | 113 | 13 | 126 | 1961 | 17(11) | |
| METSMITS95B | 65 | 15 | 80 | 1895 | 16(8) | |
| METSMITS95C | 83 | 17 | 100 | 1874 | 17(9) | |
| METSMITS95D | 399 | 72 | 472 | 1860 | 20(10) | |
| METSMITS96A | 538 | 46 | 584 | 1852 | 19(11) | |
| METSMITS96B | 546 | 51 | 598 | 1742 | 21(11) | |
| METSMITS96C | 501 | 37 | 537 | 1764 | 3(1) | present |
| METSMITS145A | 132 | 16 | 148 | 1786 | 2(1) | present |
| METSMITS145B | 166 | 9 | 175 | 1880 | 2(1) | present |
| METSMITS146A | 543 | 10 | 552 | 1823 | 2(1) | present |
| METSMITS146B | 540 | 11 | 551 | 1814 | 2(1) | present |
| METSMITS146C | 148 | 5 | 153 | 2355 | 3(1) | |
| METSMITS146D | 194 | 15 | 208 | 1693 | 2(1) | present |
| METSMITS146E | 180 | 19 | 199 | 1887 | 11(4) | present |
| METSMITS147A | 184 | 65 | 250 | 1969 | 9(3) | |
| METSMITS147B | 157 | 41 | 198 | 1911 | 11(5) | |
| METSMITS147C | 170 | 12 | 182 | 2014 | 10(3) | |
| MsmPS | | | | 1793 | 71(51) | present |
| (NC_009515) | | | | | | |
| METSMIALI | | | | 1679 | 14(9) | |
| (DSM2375) | | | | | | |
| METSMIF1 | | | | 1688 | 2(1) | |
| (DSM2374) | | | | | | |

### Example 7

### Monozygotic (MZ) Twins have Higher Concordance for Gut Methanogens than Dizygotic (DZ) Twins

A quantitative PCR (qPCR) assay of the mcrA gene was used to measure methanogens present in single fecal samples collected from 40 female MZ and 28 adult female DZ twin pairs (age 21-31 y). All were born in Missouri, although at the time they provided samples, only 29% were living in the same home and some lived >800 km apart (2). Based on a health questionnaire, all were healthy and none had a history of gastrointestinal disease including irritable bowel syndrome. Sixty-one percent were obese (BMI 30) and 7% overweight (BMI 25-30) at the time of sampling (2).

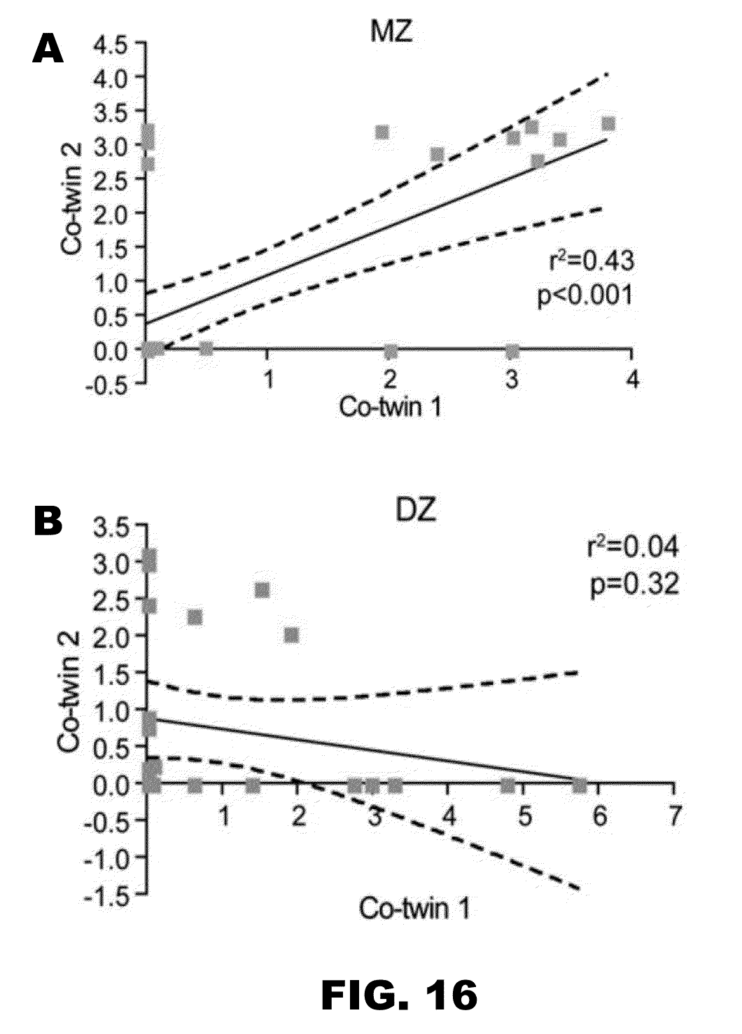

Thirty-two of the 136 individuals (23%) had levels of methanogens above our threshold for confidently calling the fecal sample “positive” (i.e., ≧4×10⁷ genome equivalents per mg of total fecal DNA), and this proportion did not vary significantly by zygosity group (P=0.59). The MZ twin pair concordance rate for carriage of methanogens was 74%, a value significantly higher than the DZ pair concordance rate (15%; P=0.009 by Breslow-Day test). In addition, there was a significantly higher degree of correlation of methanogen levels between MZ pairs by linear regression (r²=0.43, P<0.0001) than DZ pairs (r²=0.04, P=0.32), (FIGS. 16 A and B). Fecal samples were also collected from 23 of the MZ twin pairs and 12 of the DZ pairs 2 mo after the initial time point. Linear regression showed that time point 1 and time point 2 samples were highly correlated for both the presence of methanogens (r²=0.54, P<0.0001; FIG. 16C) and their levels. Neither carriage nor levels of methanogens was significantly correlated with being overweight or obese in this study population (P=0.37 and 0.38, respectively).

Thirteen samples from the initial timepoint representing 4 MZ twin pairs, 1 DZ twin pair, plus 3 other unrelated individuals that were positive for mcrA were chosen for sequencing of amplicons generated by using the mcrA primers and previously described archaeal 16S rRNA primers (n=5-10 amplicon subclones/primer set/fecal DNA sample). In 12 of the 13 samples, M. smithii was the only sequence detected by mcrA or 16S rRNA-directed PCR. In one MZ co-twin (TS17 in, Tables 24 and 25), 2 of 6 16S rRNA amplicons and 2 of 8 mcrA amplicons matched to Methanosphaera stadtmanae, a mesophilic euryarchaeota known to be present in the gut microbiota of some humans (19); the remaining amplicons generated from her fecal DNA matched to M. smithii. Her co-twin (TS16) had no detectable methanogens.

Fecal samples from 51 mothers in this study were also examined for presence of methanogens and found a similar overall degree of methanogen carriage in this population as found in their daughters (31% and 25%, respectively). Concordance for carriage of methanogens between mother and daughter (i.e., the probability that the daughter of a methanogen carrier was also a carrier, 32%) was nonsignificant (P=0.33).

TABLE 24
Summary of qPCR results for mcrA (methanogens) and
aps (SRB) in fecal samples from MZ and DZ twins

| | | | Quantification of methanogens | | | | |
|---|---|---|---|---|---|---|---|
| | | | log (mcrA) | | | | SRB |
| TS# | | zygosity | timepoint 1 | timepoint 2 | timepoint 3 | lineage | log aps |
| 1 | Co-twin 1 | MZ | 3.163 | 2.542 | 3.053 | M. smithii | 3.509 |
| 2 | Co-twin 2 | MZ | 3.293 | 3.408 | 3.901 | M. smithii | |
| 4 | Co-twin 1 | MZ | 0.000 | 0.000 | 0.000 | | 0.000 |
| 5 | Co-twin 2 | MZ | 0.000 | 0.000 | 0.000 | | 0.000 |
| 7 | Co-twin 1 | MZ | 0.000 | 0.000 | 0.000 | | 0.000 |
| 8 | Co-twin 2 | MZ | 0.000 | 0.000 | 0.000 | | 3.741 |
| 10 | Co-twin 1 | MZ | 0.000 | 0.000 | | | 0.000 |
| 11 | Co-twin 2 | MZ | 0.000 | | | | |
| 13 | Co-twin 1 | MZ | 0.000 | | | | |
| 14 | Co-twin 2 | MZ | 0.000 | | | | |
| 16 | Co-twin 1 | MZ | 0.000 | | | | 3.402 |
| 17 | Co-twin 2 | MZ | 3.243 | | | M. smithii and | |
| | | | | | | M. stadtmanae | |
| 19 | Co-twin 1 | MZ | 0.000 | | | | |
| 20 | Co-twin 2 | MZ | 0.000 | | | | |
| 22 | Co-twin 1 | MZ | 0.000 | | | | 1.744 |
| 23 | Co-twin 2 | MZ | 0.000 | | | | 0.000 |
| 25 | Co-twin 1 | MZ | 0.000 | 3.053 | | | |
| 26 | Co-twin 2 | MZ | 2.751 | 2.781 | | | |
| 28 | Co-twin 1 | MZ | 3.790 | | | | |
| 29 | Co-twin 2 | MZ | 3.344 | | | | |
| 31 | Co-twin 1 | MZ | 0.000 | | | | |
| 32 | Co-twin 2 | MZ | 0.000 | | | | |
| 34 | Co-twin 1 | MZ | 3.012 | | | M. smithii | 3.073 |
| 35 | Co-twin 2 | MZ | 3.132 | | | M. smithii | 3.356 |
| 37 | Co-twin 1 | MZ | 0.000 | | | | 0.000 |
| 38 | Co-twin 2 | MZ | 0.000 | | | | |
| 40 | Co-twin 1 | MZ | 0.000 | | | | 2.490 |
| 41 | Co-twin 2 | MZ | 0.000 | | | | |
| 43 | Co-twin 1 | MZ | 0.000 | | | | 1.958 |
| 44 | Co-twin 2 | MZ | 3.065 | | | | 0.000 |
| 46 | Co-twin 1 | MZ | 0.000 | | | | |
| 47 | Co-twin 2 | MZ | 1.126 | | | | 0.000 |
| 49 | Co-twin 1 | MZ | 0.000 | 0.000 | | | |
| 50 | Co-twin 2 | MZ | 0.000 | 0.000 | | | |
| 52 | Co-twin 1 | MZ | 0.000 | | | | |
| 53 | Co-twin 2 | MZ | 2.830 | | | | 2.615 |
| 55 | Co-twin 1 | DZ | 0.000 | | | | |
| 56 | Co-twin 2 | DZ | 0.000 | | | | |
| 58 | Co-twin 1 | MZ | 0.000 | | | | |
| 59 | Co-twin 2 | MZ | 1.582 | | | | |
| 61 | Co-twin 1 | DZ | 0.000 | 0.000 | | | 0.000 |
| 62 | Co-twin 2 | DZ | 0.052 | 0.000 | | | 0.000 |
| 64 | Co-twin 1 | MZ | 3.002 | | | | |
| 65 | Co-twin 2 | MZ | 0.000 | | | | 0.000 |
| 67 | Co-twin 1 | DZ | 0.000 | 0.000 | | | 0.000 |
| 68 | Co-twin 2 | DZ | 0.769 | 2.815 | | | 3.086 |
| 70 | Co-twin 1 | DZ | 3.270 | 3.083 | | | |
| 71 | Co-twin 2 | DZ | 0.000 | 0.858 | | | 0.000 |
| 73 | Co-twin 1 | DZ | 0.000 | 2.119 | | | 2.458 |
| 74 | Co-twin 2 | DZ | 3.109 | 3.076 | | | 0.000 |
| 76 | Co-twin 1 | MZ | 0.484 | 2.120 | | | 3.293 |
| 77 | Co-twin 2 | MZ | 0.037 | 1.894 | | | 0.000 |
| 79 | Co-twin 1 | MZ | 0.000 | | | | |
| 80 | Co-twin 2 | MZ | 0.000 | | | | 0.000 |
| 82 | Co-twin 1 | MZ | 0.000 | 0.000 | | | 2.536 |
| 83 | Co-twin 2 | MZ | 0.039 | 0.000 | | | 2.613 |
| 85 | Co-twin 1 | DZ | 0.000 | | | | 0.000 |
| 86 | Co-twin 2 | DZ | 2.995 | | | | 0.000 |
| 88 | Co-twin 1 | DZ | 0.056 | 0.103 | | | 0.000 |
| 89 | Co-twin 2 | DZ | 0.000 | 0.000 | | | 2.700 |
| 91 | Co-twin 1 | MZ | 0.000 | | | | 2.084 |
| 92 | Co-twin 2 | MZ | 0.000 | | | | 0.000 |
| 94 | Co-twin 1 | MZ | 3.212 | 3.159 | | M. smithii | |
| 95 | Co-twin 2 | MZ | 2.793 | 2.442 | | M. smithii | 2.462 |
| 97 | Co-twin 1 | DZ | 0.038 | 0.000 | | | 0.000 |
| 98 | Co-twin 2 | DZ | 0.010 | 0.000 | | | 2.044 |
| 100 | Co-twin 1 | MZ | 1.930 | 1.622 | | | 2.302 |
| 101 | Co-twin 2 | MZ | 3.215 | 0.685 | | | |
| 103 | Co-twin 1 | MZ | 0.080 | 0.000 | | | 0.000 |
| 104 | Co-twin 2 | MZ | 0.036 | 0.000 | | | 0.000 |
| 106 | Co-twin 1 | MZ | 0.000 | | | | 0.000 |
| 107 | Co-twin 2 | MZ | 0.000 | | | | |
| 109 | Co-twin 1 | DZ | 0.078 | | | | 2.006 |
| 110 | Co-twin 2 | DZ | 0.249 | | | | |
| 112 | Co-twin 1 | MZ | 0.000 | | | | 0.000 |
| 113 | Co-twin 2 | MZ | 0.000 | | | | |
| 115 | Co-twin 1 | MZ | 2.381 | 2.860 | | | |
| 116 | Co-twin 2 | MZ | 2.893 | 3.150 | | | |
| 118 | Co-twin 1 | DZ | 0.000 | | | | 0.000 |
| 119 | Co-twin 2 | DZ | 0.909 | | | | |
| 121 | Co-twin 1 | MZ | 0.000 | | | | 2.665 |
| 122 | Co-twin 2 | MZ | 0.000 | | | | |
| 124 | Co-twin 1 | DZ | 0.000 | | | | 3.002 |
| 125 | Co-twin 2 | DZ | 0.000 | | | | 3.133 |
| 127 | Co-twin 1 | DZ | 5.718 | | | M. smithii | |
| 128 | Co-twin 2 | DZ | 0.000 | | | | 0.000 |
| 130 | Co-twin 1 | MZ | 0.000 | | | | 0.000 |
| 131 | Co-twin 2 | MZ | 0.000 | | | | |
| 133 | Co-twin 1 | MZ | 0.000 | | | | |
| 134 | Co-twin 2 | MZ | 0.000 | | | | 0.000 |
| 136 | Co-twin 1 | DZ | 4.761 | | | | |
| 137 | Co-twin 2 | DZ | 0.000 | | | | 2.833 |
| 139 | Co-twin 1 | DZ | 1.890 | | | | |
| 140 | Co-twin 2 | DZ | 2.044 | | | M. smithii | 2.957 |
| 142 | Co-twin 1 | DZ | 0.000 | | | | 3.857 |
| 143 | Co-twin 2 | DZ | 0.221 | | | | 0.000 |
| 145 | Co-twin 1 | DZ | 1.502 | | | M. smithii | 4.191 |
| 146 | Co-twin 2 | DZ | 2.655 | | | M. smithii | 0.000 |
| 148 | Co-twin 1 | MZ | 0.000 | | | | |
| 149 | Co-twin 2 | MZ | 0.000 | | | | |
| 151 | Co-twin 1 | DZ | 0.000 | | | | 0.000 |
| 152 | Co-twin 2 | DZ | 3.004 | | | | 2.942 |
| 154 | Co-twin 1 | MZ | 3.388 | | | M. smithii | 0.000 |
| 155 | Co-twin 2 | MZ | 3.107 | | | M. smithii | 2.221 |
| 157 | Co-twin 1 | DZ | 1.467 | | | | 0.000 |
| 158 | Co-twin 2 | DZ | 0.000 | | | | |
| 160 | Co-twin 1 | DZ | 0.610 | | | | 0.000 |
| 161 | Co-twin 2 | DZ | 0.000 | | | | 0.000 |
| 163 | Co-twin 1 | MZ | 0.000 | | | | 0.000 |
| 164 | Co-twin 2 | MZ | 0.000 | | | | 4.550 |
| 166 | Co-twin 1 | DZ | 1.378 | | | | 0.000 |
| 167 | Co-twin 2 | DZ | 0.000 | | | | 0.000 |
| 169 | Co-twin 1 | DZ | 2.955 | | | | 3.880 |
| 170 | Co-twin 2 | DZ | 0.000 | | | | 0.000 |
| 172 | Co-twin 1 | MZ | 0.000 | | | | 3.416 |
| 173 | Co-twin 2 | MZ | 0.000 | | | | 0.000 |
| 175 | Co-twin 1 | MZ | 0.000 | | | | |
| 176 | Co-twin 2 | MZ | 0.000 | | | | 0.000 |
| 178 | Co-twin 1 | DZ | 0.613 | | | | |
| 179 | Co-twin 2 | DZ | 2.282 | | | | 1.651 |
| 181 | Co-twin 1 | DZ | 0.000 | | | | 2.505 |
| 182 | Co-twin 2 | DZ | 2.430 | | | | 4.587 |
| 184 | Co-twin 1 | MZ | 1.996 | | | | 0.000 |
| 185 | Co-twin 2 | MZ | 0.000 | | | | 0.000 |
| 187 | Co-twin 1 | DZ | 0.000 | | | | |
| 188 | Co-twin 2 | DZ | 0.000 | | | | |
| 190 | Co-twin 1 | MZ | 0.000 | | | | 3.375 |
| 191 | Co-twin 2 | MZ | 0.000 | | | | 0.000 |
| 193 | Co-twin 1 | DZ | 0.000 | | | | 3.233 |
| 194 | Co-twin 2 | DZ | 0.000 | | | | 0.000 |
| 196 | Co-twin 1 | DZ | 0.000 | | | | 2.820 |
| 197 | Co-twin 2 | DZ | 0.000 | | | | 0.000 |
| 199 | Co-twin 1 | DZ | 0.000 | | | | 2.989 |
| 200 | Co-twin 2 | DZ | 0.000 | | | | 0.000 |
| 202 | Co-twin 1 | DZ | 0.000 | | | | |
| 203 | Co-twin 2 | DZ | 0.000 | | | | |
| 205 | Co-twin 1 | DZ | 2.727 | | | | |

qPCR results are shown as log₁₀ (genome equivalents per nanogram of DNA). For mrcA, results in bold are above our threshold for calling a sample “positive”.

TABLE 25
Relative abundance of Desulfovibrio taxa (as defined
by sequencing the V2 regions of their 16S rRNA genes)

| | | | OTUs in lineages related to SRB | | | |
|---|---|---|---|---|---|---|
| | | | log (mcrA) | | | |
| TS# | | zygosity | Taxon 7973 | Taxon 12216 | Taxon 12050 | Taxon 1908 |
| 1 | Co-twin 1 | MZ | 0.000503694 | 0 | 0 | 0 |
| 2 | Co-twin 2 | MZ | 0 | 0 | 0 | 0 |
| 4 | Co-twin 1 | MZ | 0 | 0 | 0 | 0 |
| 5 | Co-twin 2 | MZ | 0 | 0 | 0 | 0 |
| 7 | Co-twin 1 | MZ | 0 | 0.000179469 | 8.97344E−05 | 0 |
| 8 | Co-twin 2 | MZ | 0 | 0.001734713 | 0.001053219 | 0 |
| 10 | Co-twin 1 | MZ | 0 | 0 | 0 | 0 |
| 11 | Co-twin 2 | MZ | 0 | 0 | 0 | 0 |
| 13 | Co-twin 1 | MZ | 0 | 0.001230769 | 0.001107692 | 0 |
| 14 | Co-twin 2 | MZ | 0 | 0.000129266 | 0 | 0 |
| 16 | Co-twin 1 | MZ | 0 | 0 | 0.002105263 | 0 |
| 17 | Co-twin 2 | MZ | 0 | 0 | 0 | 0 |
| 19 | Co-twin 1 | MZ | 0 | 0 | 0 | 0 |
| 20 | Co-twin 2 | MZ | 0 | 0 | 0 | 0 |
| 22 | Co-twin 1 | MZ | 0 | 0 | 0 | 0 |
| 23 | Co-twin 2 | MZ | 0 | 0 | 0 | 0 |
| 25 | Co-twin 1 | MZ | 0 | 0 | 0 | 0 |
| 26 | Co-twin 2 | MZ | 6.27983E−05 | 0 | 0 | 0 |
| 28 | Co-twin 1 | MZ | 0 | 0 | 0 | 0 |
| 29 | Co-twin 2 | MZ | 0 | 0 | 0 | 0 |
| 31 | Co-twin 1 | MZ | 0 | 0 | 0 | 0 |
| 32 | Co-twin 2 | MZ | 0 | 0.001546278 | 0.001546278 | 0 |
| 34 | Co-twin 1 | MZ | 0 | 0 | 0.003594536 | 0 |
| 35 | Co-twin 2 | MZ | 0 | 0 | 0.004326123 | 0 |
| 37 | Co-twin 1 | MZ | 0 | 0 | 0 | 0 |
| 38 | Co-twin 2 | MZ | 0 | 0 | 0 | 0 |
| 40 | Co-twin 1 | MZ | | | | |
| 41 | Co-twin 2 | MZ | | | | |
| 43 | Co-twin 1 | MZ | 0 | 0 | 0 | 0 |
| 44 | Co-twin 2 | MZ | 0 | 0 | 0 | 0 |
| 46 | Co-twin 1 | MZ | | | | |
| 47 | Co-twin 2 | MZ | | | | |
| 49 | Co-twin 1 | MZ | 0 | 0 | 0 | 0 |
| 50 | Co-twin 2 | MZ | 0 | 0 | 0 | 0 |
| 52 | Co-twin 1 | MZ | | | | |
| 53 | Co-twin 2 | MZ | | | | |
| 55 | Co-twin 1 | DZ | 0 | 0 | 0 | 0 |
| 56 | Co-twin 2 | DZ | 0 | 0 | 0 | 0 |
| 58 | Co-twin 1 | MZ | | | | |
| 59 | Co-twin 2 | MZ | | | | |
| 61 | Co-twin 1 | DZ | 0 | 0 | 0 | 0 |
| 62 | Co-twin 2 | DZ | 0 | 0 | 0 | 0 |
| 64 | Co-twin 1 | MZ | 0 | 0 | 0 | 0 |
| 65 | Co-twin 2 | MZ | 0 | 0 | 0 | 0 |
| 67 | Co-twin 1 | DZ | 0 | 0 | 0 | 0 |
| 68 | Co-twin 2 | DZ | 0 | 0 | 0 | 0.001277139 |
| 70 | Co-twin 1 | DZ | 0 | 0 | 0 | 0 |
| 71 | Co-twin 2 | DZ | 0 | 0 | 0 | 0 |
| 73 | Co-twin 1 | DZ | 0 | 0 | 0 | 0 |
| 74 | Co-twin 2 | DZ | 0 | 0 | 0 | 0 |
| 76 | Co-twin 1 | MZ | 0 | 0 | 0 | 0.000676361 |
| 77 | Co-twin 2 | MZ | 0 | 0 | 0 | 0 |
| 79 | Co-twin 1 | MZ | | | | |
| 80 | Co-twin 2 | MZ | | | | |
| 82 | Co-twin 1 | MZ | 0 | 0 | 0 | 0 |
| 83 | Co-twin 2 | MZ | 0 | 0 | 0.000645161 | 0 |
| 85 | Co-twin 1 | DZ | 0 | 0 | 0 | 0 |
| 86 | Co-twin 2 | DZ | 0 | 0 | 0 | 0 |
| 88 | Co-twin 1 | DZ | 0 | 0 | 0 | 0 |
| 89 | Co-twin 2 | DZ | 0 | 0 | 0 | 0.000959233 |
| 91 | Co-twin 1 | MZ | 0 | 0 | 0 | 0 |
| 92 | Co-twin 2 | MZ | 0 | 0 | 0 | 0 |
| 94 | Co-twin 1 | MZ | 0 | 0 | 0 | 0.008077544 |
| 95 | Co-twin 2 | MZ | 0 | 0 | 0 | 0.011243851 |
| 97 | Co-twin 1 | DZ | 0 | 0 | 0 | 0 |
| 98 | Co-twin 2 | DZ | 0 | 0 | 0 | 0 |
| 100 | Co-twin 1 | MZ | 0 | 0 | 0 | 0 |
| 101 | Co-twin 2 | MZ | | | | |
| 103 | Co-twin 1 | MZ | 0 | 0 | 0 | 0 |
| 104 | Co-twin 2 | MZ | 0 | 0 | 0 | 0 |
| 106 | Co-twin 1 | MZ | 0 | 0 | 0 | 0 |
| 107 | Co-twin 2 | MZ | 0 | 0 | 0 | 0 |
| 109 | Co-twin 1 | DZ | 0 | 0 | 0.002912621 | 0 |
| 110 | Co-twin 2 | DZ | 0 | 0 | 0 | 0 |
| 112 | Co-twin 1 | MZ | | | | |
| 113 | Co-twin 2 | MZ | | | | |
| 115 | Co-twin 1 | MZ | 0 | 0 | 0 | 0.002368733 |
| 116 | Co-twin 2 | MZ | 0 | 0 | 0.003847563 | 0.001832173 |
| 118 | Co-twin 1 | DZ | 0 | 0 | 0 | 0 |
| 119 | Co-twin 2 | DZ | 0 | 0 | 0 | 0 |
| 121 | Co-twin 1 | MZ | | | | |
| 122 | Co-twin 2 | MZ | | | | |
| 124 | Co-twin 1 | DZ | 0 | 0 | 0 | 0 |
| 125 | Co-twin 2 | DZ | 0 | 0 | 0.00084317 | 0 |
| 127 | Co-twin 1 | DZ | 0.000312305 | 0 | 0 | 0 |
| 128 | Co-twin 2 | DZ | 0 | 0 | 0 | 0 |
| 130 | Co-twin 1 | MZ | 0 | 0 | 0 | 0 |
| 131 | Co-twin 2 | MZ | 0 | 0 | 0 | 0 |
| 133 | Co-twin 1 | MZ | 0 | 0 | 0 | 0 |
| 134 | Co-twin 2 | MZ | 0 | 0 | 0 | 0 |
| 136 | Co-twin 1 | DZ | 0 | 0 | 0 | 0.003103448 |
| 137 | Co-twin 2 | DZ | 0 | 0 | 0.001086957 | 0 |
| 139 | Co-twin 1 | DZ | 0 | 0 | 0.000363504 | 0 |
| 140 | Co-twin 2 | DZ | 0 | 0 | 0.002235469 | 0.002980626 |
| 142 | Co-twin 1 | DZ | 0 | 0 | 0 | 0 |
| 143 | Co-twin 2 | DZ | 0 | 0 | 0 | 0 |
| 145 | Co-twin 1 | DZ | 0 | 0 | 0 | 0 |
| 146 | Co-twin 2 | DZ | 0 | 0 | 0 | 0 |
| 148 | Co-twin 1 | MZ | 0 | 0.001706193 | 0.001023716 | 0 |
| 149 | Co-twin 2 | MZ | 0 | 0 | 0 | 0 |
| 151 | Co-twin 1 | DZ | 0 | 0 | 0 | 0 |
| 152 | Co-twin 2 | DZ | 0 | 0 | 0 | 0 |
| 154 | Co-twin 1 | MZ | | | | |
| 155 | Co-twin 2 | MZ | 0 | 0 | 0 | 0 |
| 157 | Co-twin 1 | DZ | | | | |
| 158 | Co-twin 2 | DZ | | | | |
| 160 | Co-twin 1 | DZ | 0 | 0 | 0.001730104 | 0 |
| 161 | Co-twin 2 | DZ | 0 | 0 | 0 | 0 |
| 163 | Co-twin 1 | MZ | 0 | 0 | 0 | 0 |
| 164 | Co-twin 2 | MZ | 0 | 0 | 0 | 0 |
| 166 | Co-twin 1 | DZ | 0 | 0 | 0 | 0 |
| 167 | Co-twin 2 | DZ | 0 | 0 | 0 | 0 |
| 169 | Co-twin 1 | DZ | 0 | 0 | 0 | 0.000481696 |
| 170 | Co-twin 2 | DZ | 0 | 0 | 0 | 0 |
| 172 | Co-twin 1 | MZ | | | | |
| 173 | Co-twin 2 | MZ | | | | |
| 175 | Co-twin 1 | MZ | | | | |
| 176 | Co-twin 2 | MZ | | | | |
| 178 | Co-twin 1 | DZ | 0 | 0 | 0 | 0 |
| 179 | Co-twin 2 | DZ | 0 | 0 | 0 | 0 |
| 181 | Co-twin 1 | DZ | 0 | 0 | 0 | 0 |
| 182 | Co-twin 2 | DZ | 0 | 0 | 0 | 0.012687428 |
| 184 | Co-twin 1 | MZ | 0 | 0 | 0 | 0 |
| 185 | Co-twin 2 | MZ | 0 | 0 | 0 | 0 |
| 187 | Co-twin 1 | DZ | | | | |
| 188 | Co-twin 2 | DZ | | | | |
| 190 | Co-twin 1 | MZ | 0 | 0 | 0 | 0.000644122 |
| 191 | Co-twin 2 | MZ | 0 | 0 | 0 | 0 |
| 193 | Co-twin 1 | DZ | 0 | 0 | 0.002008032 | 0 |
| 194 | Co-twin 2 | DZ | 0 | 0 | 0 | 0 |
| 196 | Co-twin 1 | DZ | | | | |
| 197 | Co-twin 2 | DZ | | | | |
| 199 | Co-twin 1 | DZ | | | | |
| 200 | Co-twin 2 | DZ | | | | |
| 202 | Co-twin 1 | DZ | | | | |
| 203 | Co-twin 2 | DZ | | | | |

### Example 8

### Co-Occurance Between M. smithii and Bacterial Taxa

The qPCR results suggest that host genetic factors, including factors that influence the representation of potential syntrophic partners, may play a role in carriage of methanogens. In contrast, the study of Florin et al. (17), which used methane breath tests, showed no significant differences in concordance between young adolescent Australian MZ and DZ twin pairs. The difference could be explained if environmental factors play a dominant role in determining whether methanogens are acquired early in life, whereas persistent carriage in later life is determined by a variety of host factors. Such factors range from human genotype to the presence or absence of bacterial taxa that can collaborate or compete with the methanogens.

A role for host factors in determining carriage of methanogens is supported by previous studies of nonhuman primates. Methanogens were present in the gut microbiota of some primate phylogenetic lineages but not others; however, these patterns did not follow any identifiable features of gut physiology or morphology, nor behavior or diet (20). Another study that examined the distribution of methanogens within the guts of 253 vertebrate species found “methanogenic branches” of the host phylogenetic tree [i.e., branches containing ruminants (bovidae, cervidae, giraffidae) and “nonmethanogenic” branches (felidae, canidae, and ursidae)]. As with the primate study, the methane-producing groups could not be distinguished from the methane-negative groups based on their diets or features of their gut structure/physiology (21).

To understand whether methanogen carriage might be determined, in part, by the presence or absence of bacterial taxa that can collaborate or compete with the methanogens, the co-occurrence patterns between methanogens and sulfate-reducing bacteria (SRB) was investigated. SRB, which can use H₂ as an electron donor to generate hydrogen sulfide (H₂S) through anaerobic sulfate respiration, may show positive associations with methanogens if a hydrogen economy is more important in some individuals than others, or negative associations due to competition for H₂. Positive associations between SRB and methanogens might also occur because of syntrophy, because some methanogens and SRB can grow syntrophically on lactate, with the methanogen removing H₂ generated by the SRB (22, 23). Therefore, it was determined whether SRB and methanogens had nonrandom codistribution patterns by SRB-directed qPCR assays of 87 fecal samples from the MZ and DZ twin pairs. The aps gene encodes adenosine-5′-phosphosulfate reductase, a key enzyme that catalyzes activation and then reduction of sulfate to sulfite (24). We chose aps as a target for a qPCR assay that used previously described and validated primers (25). Forty-five percent of the samples were positive for SRB (threshold of detection defined as ≈4×10⁷ genome equivalents per mg of fecal DNA). The concordance rate for sulfate reducers was not significant for either MZ or DZ co-twins (31% and 27%, Tables 24 and 25). A logistic regression was performed to determine whether a higher level of mcrA is predictive of the presence of aps or vice versa. No statistically significant relationship was identified in either comparison (P=0.10 and 0.07).

A general search for bacterial Operational Taxonomic Units (OTUs) that had positive or negative associations with M. smithii was also performed, using sequences generated from multiplex pyrosequencing of the V2 variable region of bacterial 16S rRNA genes from these same fecal samples (2). The raw sequences from this prior study were now processed by using the PyroNoise algorithm to remove sequencing noise (26), as implemented in QIIME (27). Using UCLUST (28), the denoised sequences were further divided into OTUs that each shared ≧96% nucleotide sequence identity (a value slightly more permissive than the 97% ID threshold typically used to denote a microbial species). The most abundant sequence within each of the resulting 12,833 OTUs was then selected as a representative of that OTU. Because some of the individuals in the study were sampled multiple times, one sample per individual was randomly selected. For each of the 607 OTUs that were found in at least 10 of the samples for which there was mcrA qPCR data, an ANOVA was performed to determine whether the OTU relative abundance was significantly different in methanogen-positive and -negative individuals. Associated presence/absence patterns were also checked for by using the G-test of independence (an OTU was scored as present if it was observed one or more times). The resulting P values were corrected for multiple comparisons by using the Bonferroni correction (multiplied by 607; the number of comparisons) and the false discovery rate (FDR) method (multiplied by the number of comparisons divided by the P value rank).

Twenty-two OTUs had significantly different relative abundances in mcrA-positive versus negative individuals (P<0.05 using ANOVA with the FDR correction). Of these 22 OTUs, 21 were more abundant in samples where methanogens were present, whereas one OTU was less abundant. The G-test identified five significant OTUs (P<0.05 with FDR correction), and 4 of these 5 were also significant as judged by ANOVA. All G-test-identified associations were positive. Thus, the two statistical tests together identified 22 positively associated OTUs (Table 26) and one negatively associated OTU.

To investigate the phylogenetic relationships of these OTUs to each other, and to bacterial isolates and lineages with known biological properties, parsimony insertion was used to add a representative sequence for each significant OTU into the Greengenes coreset tree (29) in the Arb software package (30). Because the closest relatives of the OTUs were mostly from other culture-independent metagenomic studies, 16S rRNA sequences were also inserted into the tree that were from well-characterized bacteria, including 16S rRNAs from fully sequenced genomes deposited in KEGG or sequenced through the Human Gut Microbiome Initiative (HGMI; http://genome.wustl.edu/genomes/list/human_gut_microbiome/), and 16S rRNA sequences from related organisms with known properties that were identified by using BLAST searches against the National Center for Biotechnology Information nonredundant database. To look for evidence of whether relatives of the OTUs were capable of growing in pure culture, the 16S rRNA sequences were also BLASTed against sequences in the RDP (31) that were marked as being from cultured bacterial isolates.

Remarkably, 20 of the 22 positively associated OTUs were members of the class Clostridiales (Firmicutes phylum). These 20 OTUs binned into five broad groups that were scattered throughout the class, including members of the three main clusters found in the human gut (clusters I, IV, and XIVa).

The group most positively associated with M. smithii was a lineage within Clostridia cluster IV that contains members of the genera Oscillospira and Sporobacter (Table 26; note that this group had the four most significant OTUs according to the ANOVA test). Two of these OTUs are highly related to Oscillospira guilliermondii, an as yet uncultured, large, and morphologically conspicuous organism found in ruminants (32, 33). The most closely related cultured isolate that we could find for any of these OTUs is Sporobacter termitidis, a hydrogen-consuming acetogen from the termite gut (34).

Two of the positively associated OTUs are members of Clostridia cluster

XIVa. The closest isolate with a sequenced genome was Blautia hydrogenotrophica, a hydrogen-consuming homoacetogen from the human gut, although the percent identity across the lanemasked V2 region was low (89-93%) and more closely related organisms to B. hydrogenotrophica are known not to be acetogens. Whether the Sporobacter and B. hydrogenotrophica-related OTUs are acetogens cannot be determined by using 16S rRNA sequences alone, because acetogenesis is only inconsistently associated with 16S rRNA-defined phylotypes (35). However, the relationship suggests that some OTUs may co-occur with methanogens because they are homoacetogens and have a shared preference for hydrogen. Nonetheless, the OTU most related to B. hydrogenotrophica in this analysis (99% ID) did not show significant co-occurrence with M. smithii (uncorrected P value=0.38), indicating that not all homoacetogens in the human co-occur with M. smithii because of this preference for hydrogen.

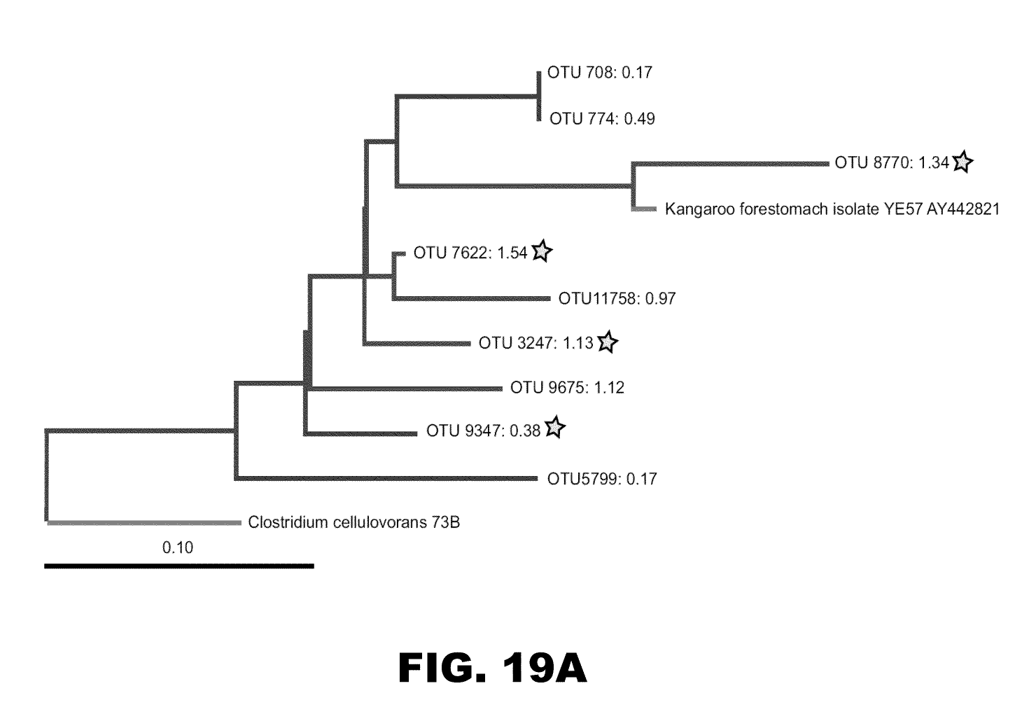

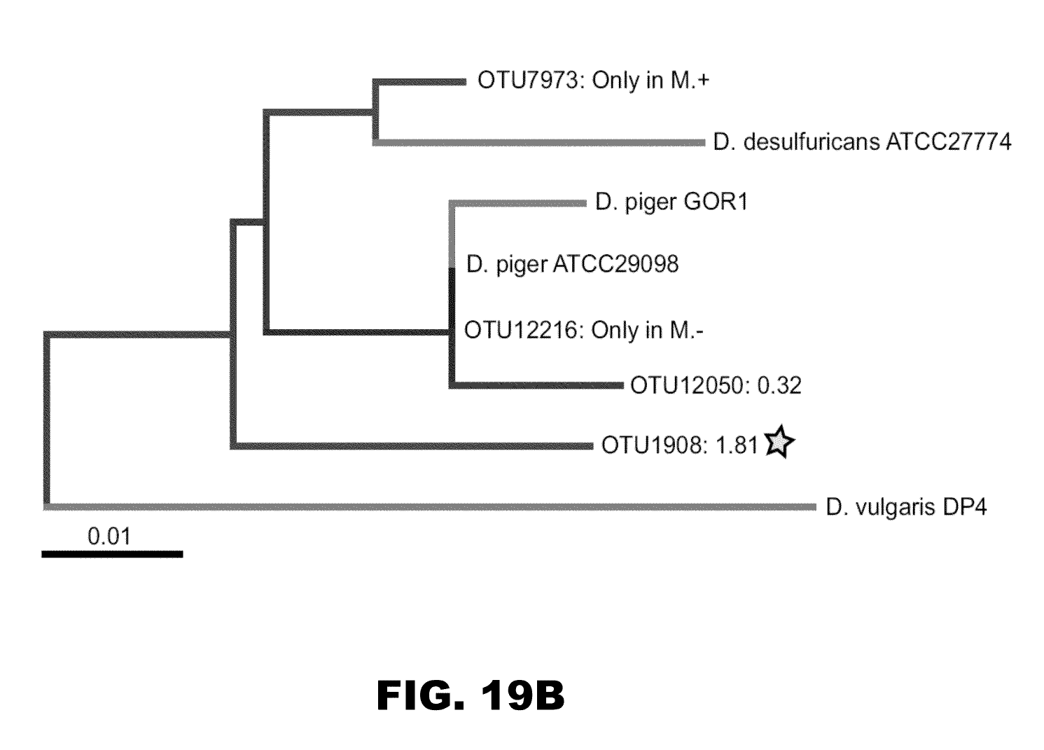

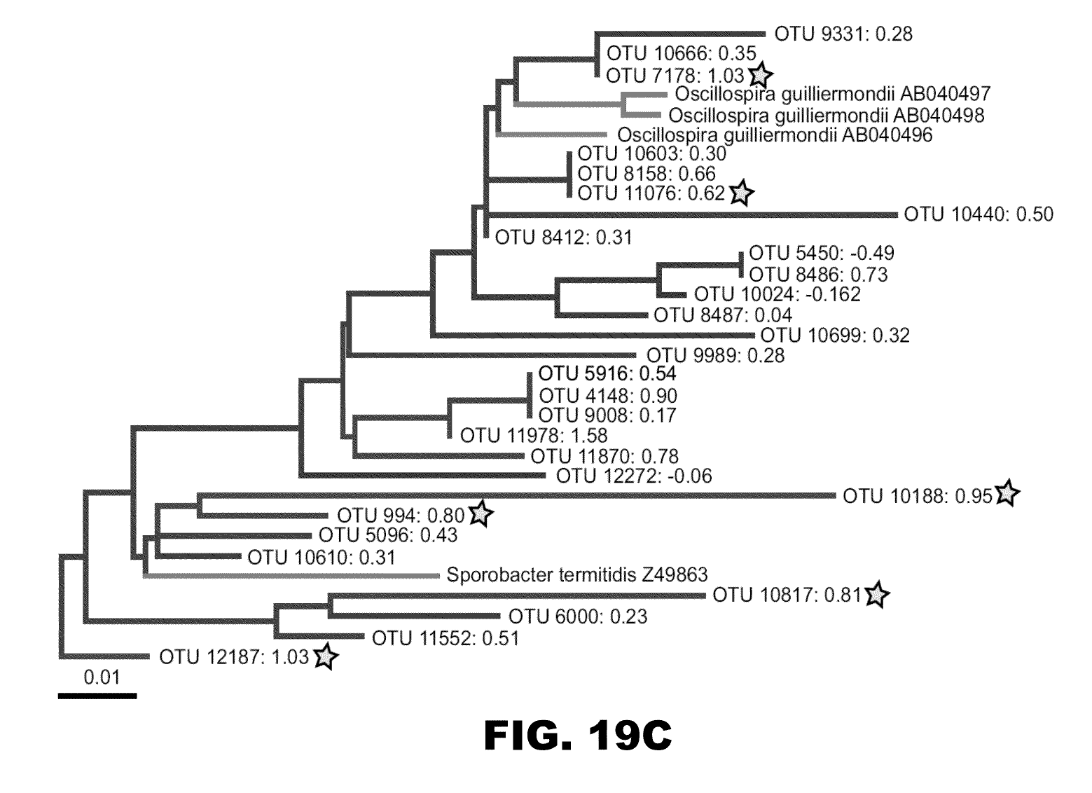

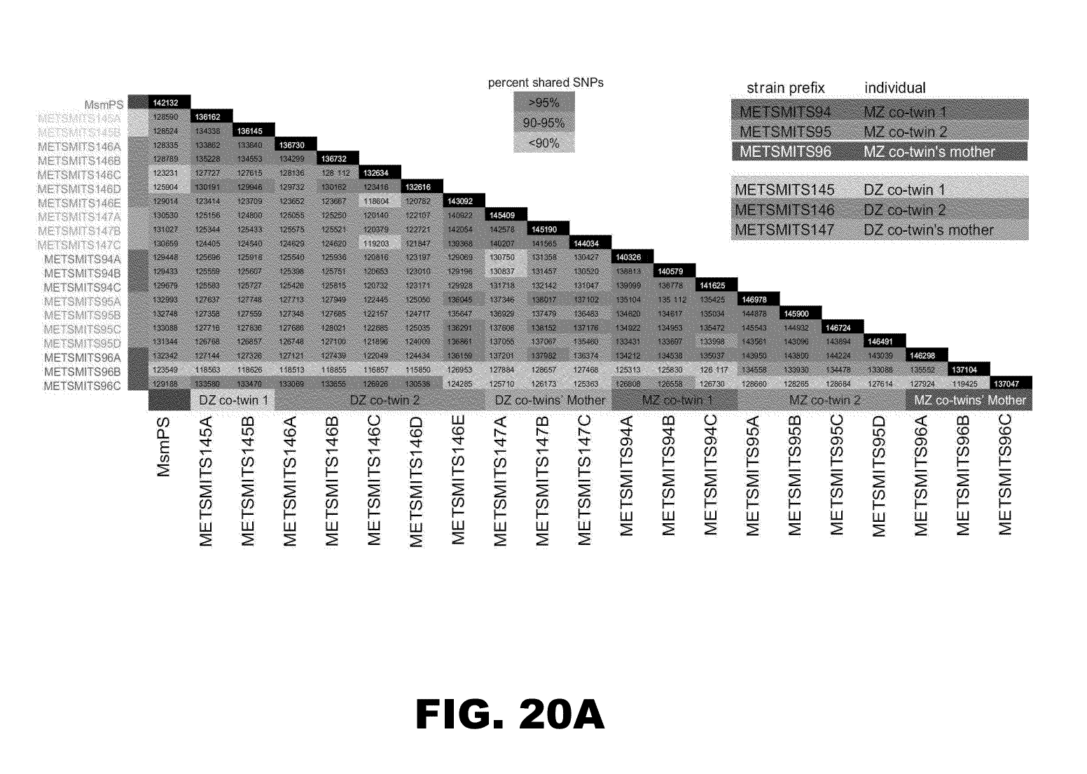

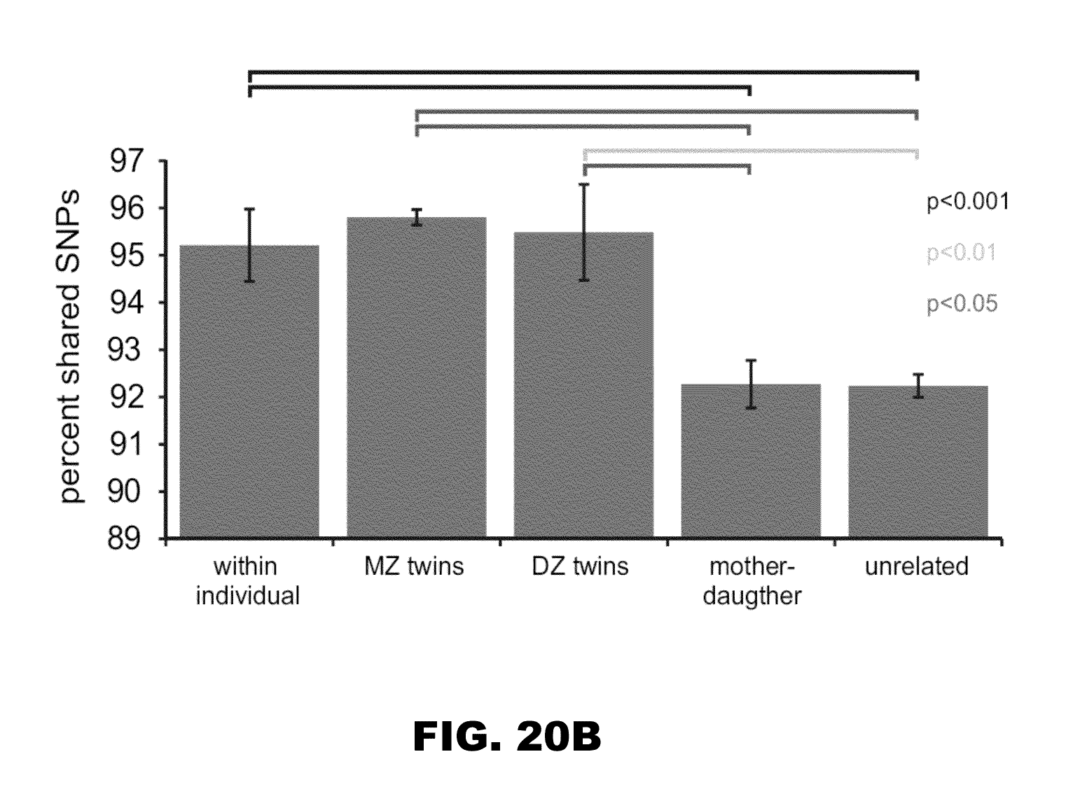

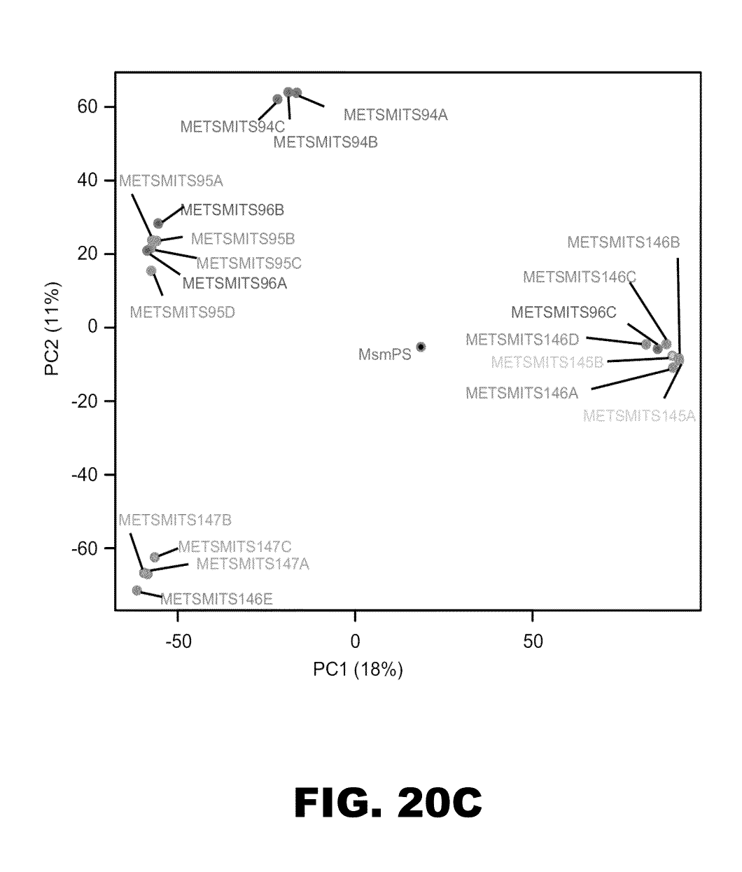

Because members of the SRB can produce and consume H₂, OTUs in the dataset that were in this group were of specific interest. Eighty-two of 281 fecal samples (29%) from the 16S rRNA analysis of these twin pairs (including additional fecal samples for which we did not obtain mcrA data) (2) had OTUs that were within the SRB Glade (FIG. 19B). The actual prevalence of SRB is likely higher, because the samples were not exhaustively sequenced. Phylogenetic comparison indicated that these OTUs represented Desulfovibrio piger in 41 (14.6%) of the samples, Desulfovibrio desulfuricans in 10 samples (3.6%), and an additional taxon (1908) in 38 samples (13.5%) that was only distantly related to cultured isolates (Table 26 and FIG. 19). Although significant associations were not detected with the SRB-specific qPCR, OTU 1908 showed a significantly positive association with methanogens (Table 26). The abundant OTU representing D. piger (OTU 12050) did not have statistically significant co-occurrence with methanogens (FIG. 19), and the three different types of SRB did not significantly co-occur with each other. The differing distribution patterns of the three different SRB species, coupled with the smaller number of fecal samples for which we had aps compared with mcrA qPCR data, likely contributed to our inability to detect a significant association between methanogens and SRB with the aps qPCR assay.

The concentration of H₂ in the gut lumen can vary over a wide range in healthy individuals (from 0.17% to 49% in a study of 11 subjects; ref. 36). Levels of H₂ in the distal gut reflect the dynamic interplay between microbial production and consumption. One of the co-occurring groups within the Clostridiales may produce abundant amounts of hydrogen. Specifically, two of the positively associated OTUs in the Clostridiales family mapped to a Glade that included isolate Rennanqilyf3, which was recovered from activated sludge by using a procedure designed to retrieve bacteria with particularly high yields of hydrogen (37). This isolate performs ethanol-type fermentation with glucose as an optimal carbon source for hydrogen production; however, its hydrogen production capacity varies with hydrogen concentration and pH. Thus, methanogen (M. smithii) abundance may be in part regulated by the presence of bacterial lineages that are efficient hydrogen producers. To our knowledge, no cultured isolates are available for members of this lineage from the gut.

Some of the OTUs that are positively associated with methanogens are quite distant from any cultured relatives (ribotypes): This observation is intriguing, because it suggests that syntrophic relationships may inhibit them from growing in monoculture. For example, four OTUs grouped in a Glade of the Clostridiales family that is dominated by relatives identified in culture-independent studies of cellulose-degrading gut environments where methanogens also reside (e.g., termite gut and cow rumen) (Gut Clone Group; Table 26 and FIG. 19A). The closest organism with a sequenced genome was only very distantly related, with a 78-86% ID over the lanemasked V2 region of rRNA. A BLAST search against the cultured component of the RDP revealed one successful attempt to culture a relative of one of these four OTUs (95% ID) from the forestomach of the kangaroo (38). However, this cultured isolate was much more distant from the other three co-occurring OTUs in this Glade, and there are no reported cultured relatives for any of these four OTUs from the human gut. Three co-occurring OTUs fell within the Catabacter lineage. The closest cultured isolate, Catabacter sp. YIT12065, is only 82-92% identical to these co-occurring OTUs; very little is known about this isolate's biology. The presence of obligate syntrophs for methanogens in the human gut would not be surprising, because they are known to exist in other environments, such as sludge (39, 40).

Unfortunately, the lack of cultured relatives for these OTUs limits the ability to more fully interpret the co-occurrence results, because knowledge is lacking about their biological properties. Targeted attempts to culture gut bacteria in the presence of M. smithii as well as targeted attempts to obtain and sequence their genomes from mixed populations should help to elucidate their functional relationships with human gut methanogens.

TABLE 26
Bacterial taxa that co-occur with methanogens

| | Related bacteria | ANOVA p-value | | | G-test p-value | | | |
|---|---|---|---|---|---|---|---|---|
| OTU # | (% identity) | Raw | Bonferroni | fdr | Raw | Bonferroni | fdr | rank |

Delta Proteobacteria; Desulfovibrio;

| | | | | | | | |
|---|---|---|---|---|---|---|---|
| 1908 | D. piger (87.4) | 4.07E−04 | 2.47E−01 | 2.24E−02 | NS | NS | 11 |
| | D. desulfuricans (90) | | | | | | |

Bacteroidetes; Alistipes;

| | | | | | | | |
|---|---|---|---|---|---|---|---|
| 4544 | Alistipes putridinis (91.6) | 7.10E−04 | 4.31E−01 | 2.87E−02 | NS | NS | 15 |

Firmicutes; Clostridiales; Cluster IV; Sporobacter/Oscillospira;

| | | | | | | | | |
|---|---|---|---|---|---|---|---|---|
| 994 | Oscillospira guilliermondi | 3.07E−06 | 1.86E−03 | 1.86E−03 | 7.48E−05 | 4.54E−02 | 2.27E−02 | 1 |
| | (94) | | | | | | | |
| | Sporobacter termitidis (89) | | | | | | | |
| 7178 | Oscillospira guilliermondi | 1.80E−05 | 1.09E−02 | 5.45E−03 | | NS | NS | 2 |
| | (95.6) | | | | | | | |
| | Sporobacter termitidis | | | | | | | |
| | (89.5) | | | | | | | |
| 11076 | Oscillospira guilliermondi | 4.12E−05 | 2.50E−02 | 8.33E−03 | | NS | NS | 3 |
| | (96) | | | | | | | |
| | Sporobacter termitidis | | | | | | | |
| | (89.7) | | | | | | | |
| 12187 | Oscillospira guilliermondi | 5.46E−05 | 3.32E−02 | 8.29E−03 | 7.55E−05 | 4.58E−02 | 1.53E−02 | 4 |
| | (93) | | | | | | | |
| | Sporobacter termitidis (93) | | | | | | | |
| 10817 | Oscillospira guilliermondi | 9.70E−04 | 5.89E−01 | 3.10E−02 | | NS | NS | 19 |
| | (89) | | | | | | | |
| | Sporobacter termitidis | | | | | | | |
| | (88.5) | | | | | | | |
| 10188 | Oscillospira guilliermondi | 1.06E−03 | 6.43E−01 | 3.22E−02 | 2.44E−04 | 1.48E−01 | 2.96E−02 | 20 |
| | (92.6) | | | | | | | |
| | Sporobacter termitidis (88) | | | | | | | |

Firmicutes; Clostridiales; Cluster IV; Rennanqily;

| | | | | | | | | |
|---|---|---|---|---|---|---|---|---|
| 10297 | Rennanqilyf3_AY363375 | 2.82E−04 | 1.71E−01 | 2.14E−02 | | NS | NS | 8 |
| | (91.9) | | | | | | | |
| 10741 | Rennanqilyf3_AY363375 | 3.03E−04 | 1.84E−01 | 2.05E−02 | 2.28E−04 | 1.39E−01 | 3.46E−02 | 9 |
| | (87) | | | | | | | |

Firmicutes; Clostridiales; Cluster IV; Anaerotruncus;

| | | | | | | | |
|---|---|---|---|---|---|---|---|
| 10014 | Clostridium methylpentosum | 2.07E−04 | 1.26E−01 | 1.79E−02 | NS | NS | 7 |
| | (92) | | | | | | |
| | Anaerotruncus colihominis | | | | | | |
| | (91) | | | | | | |
| 8310 | Clostridium methylpentosum | 6.13E−04 | 3.72E−01 | 2.86E−02 | NS | NS | 13 |
| | (92.6) | | | | | | |
| | Anaerotruncus colihominis | | | | | | |
| | (92) | | | | | | |

Firmicutes; Clostridiales; Catabacter;

| | | | | | | | | |
|---|---|---|---|---|---|---|---|---|
| 3231 | Catabacter sp. YIT12065 | 1.48E−04 | 8.98E−02 | 1.80E−02 | | NS | NS | 5 |
| | AB490809 (85) | | | | | | | |
| 6560 | Catabacter sp. YIT12065 | 1.56E−04 | 9.46E−02 | 0.016 | | NS | NS | 6 |
| | AB490809 (92) | | | | | | | |
| 4838 | Catabacter sp. YIT12065 | 9.07E−03 | 5.50E+00 | 1.34E−01 | 6.71E−05 | 4.07E−02 | 4.07E−02 | 41 |
| | AB490809 (81.9) | | | | | | | |

Firmicutes; Clostridiales; Cluster I; Gut Clone Group;

| | | | | | | | |
|---|---|---|---|---|---|---|---|
| 3247 | Clostridium cellulovorans | 3.13E−04 | 1.90E−01 | 1.90E−02 | NS | NS | 10 |
| | (83.3) | | | | | | |
| | Kangaroo forestomach | | | | | | |
| | isolate YE57 AY442821 | | | | | | |
| | (86.5) | | | | | | |
| 7622 | Clostridium cellulovorans | 7.32E−04 | 4.44E−01 | 2.78E−02 | NS | NS | 16 |
| | (85.7) | | | | | | |
| | Kangaroo forestomach | | | | | | |
| | isolate YE57 AY442821 | | | | | | |
| | (83.5) | | | | | | |
| 9347 | Clostridium cellulovorans | 9.21E−04 | 5.59E−01 | 3.11E−02 | NS | NS | 18 |
| | (81.7) | | | | | | |
| | Kangaroo forestomach | | | | | | |
| | isolate YE57 AY442821 | | | | | | |
| | (83) | | | | | | |
| 8770 | Clostridium cellulovorans | 1.41E−03 | 8.59E−01 | 4.10E−02 | NS | NS | 21 |
| | (78.4) | | | | | | |
| | Kangaroo forestomach | | | | | | |
| | isolate YE57 AY442821 | | | | | | |
| | (94.9) | | | | | | |

Firmicutes; Clostridiales; Cluster XIVa;

| | | | | | | | |
|---|---|---|---|---|---|---|---|
| 2502 | Blautia hydrogenotrophica | 6.37E−04 | 3.87E−01 | 2.76E−02 | NS | NS | 14 |
| | (92.5) | | | | | | |
| 4531 | Blautia hydrogenotrophica | 1.75E−03 | 1.06E+00 | 4.82E−02 | NS | NS | 22 |
| | (89) | | | | | | |
| 4683 | Coprococcus eutactus | 7.72E−04 | 4.69E−01 | 2.76E−02 | NS | NS | 17 |
| | (98.9) | | | | | | |

OTUs found to be significantly co-occurring with methanogens are shown, together with information about their phylogeny, the percent identity of the V2 regions of their 16S rRNA gene sequence with previously described related bacterial taxa, a P value for co-occurrence as defined by ANOVA, and corrected for multiple hypothesis testing (false discovery rate correction). Significant P values are noted in red, whereas insignificant values are shown in black or denoted with “NS.” The rank is for the ANOVA P values. G-test P values are only given for the ones that were significant after applying the FDR correction. Related isolates are followed by their percent nucleotide sequence identity (% ID) to the listed organism over the V2 region of their 16S rRNA genes (after the Lane mask for hypervariable positions was applied).

### Example 9

### Analysis of the Pan-Genome of M. smithii

It was reasoned that one approach for further characterizing factors that affect M. smithii colonization of the human gut would be to develop a method for isolating strains from frozen fecal samples obtained from twins and their mothers, sequencing their genomes, and performing RNA-Seq to evaluate strain-level variations in patterns of gene expression during growth under varying levels of hydrogen and formate.

The method that was developed for recovering M. smithii from frozen fecal samples is described above. A total of 20 strains were isolated from two families: one consisting of a MZ twin pair and their mother and the other a DZ twin pair and their mother (n=2-5 strains isolated and sequenced per individual). Deep draft genome assemblies were generated by using reads produced by Illumina GA-IIx and 454 sequencers. Table 23 describes the details of genome coverage and of the assembly statistics. Assembled genomes were aligned by using Mauve (41), which iteratively reordered contigs based on the finished genome sequence of the M. smithii type strain PS (42). Table 23 also provides information about previously generated, deep draft assemblies of the genomes of two other M. smithii type strains obtained from culture collections (42).

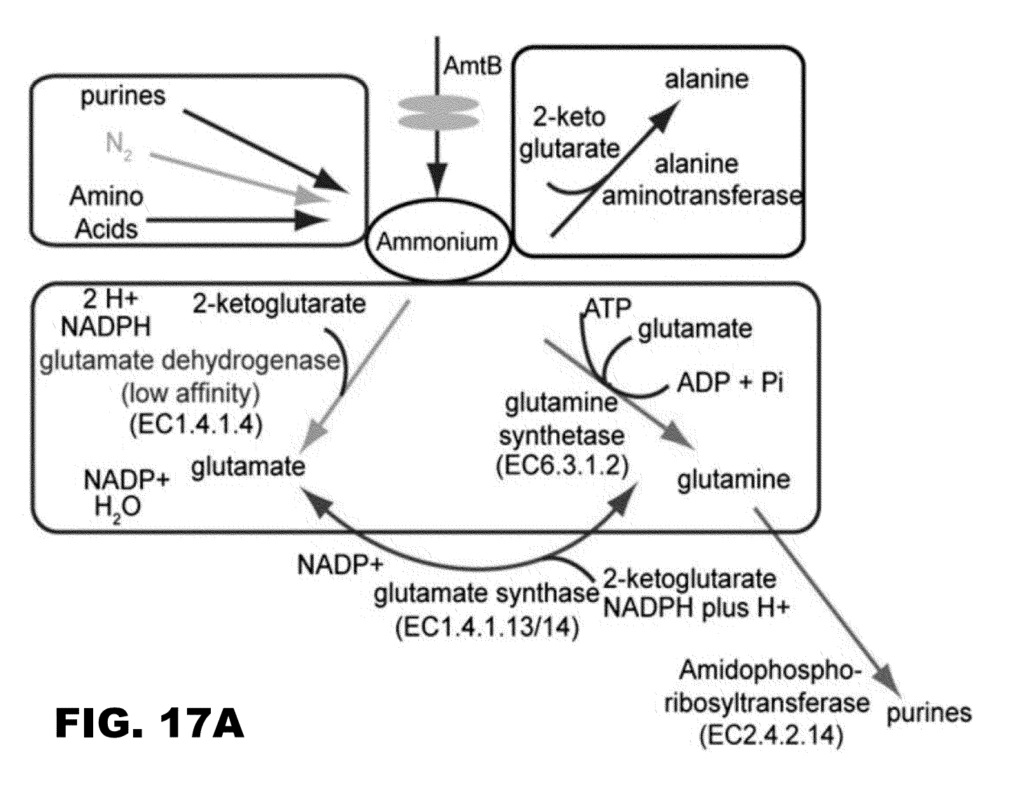

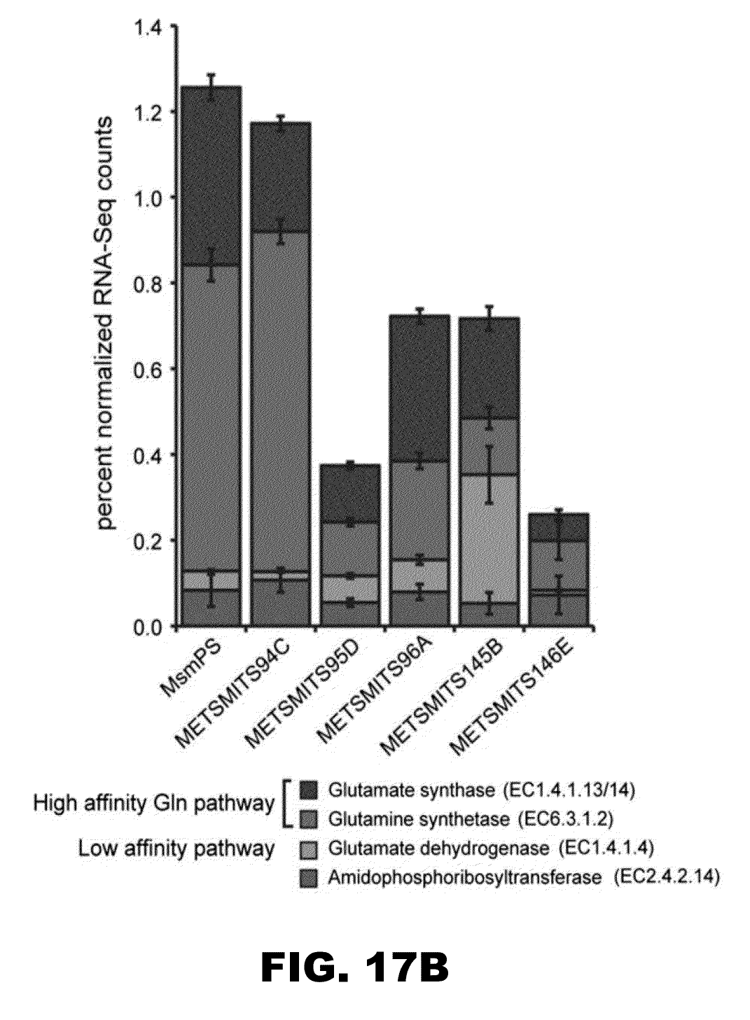

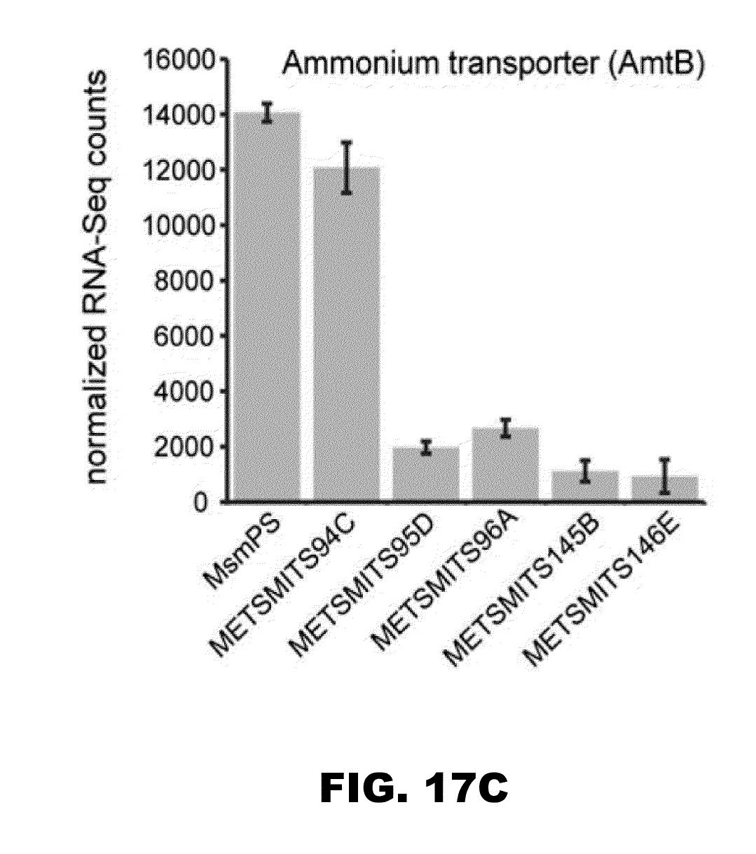

On average, any two strains shared 92.96±6.5% of their single nucleotide polymorphisms (SNPs) [129,112±6,322 (mean±SD)]. A binary table of the presence or absence of a SNP was subsequently generated, a distance matrix was calculated, and a principal components analysis (PCA) was performed (FIGS. 17A and C). The PCA showed that strains from the same individual and strains from co-twins clustered together. Both MZ and DZ co-twins shared significantly more SNPs in their strains than with strains from their mothers or unrelated individuals (FIG. 17B).

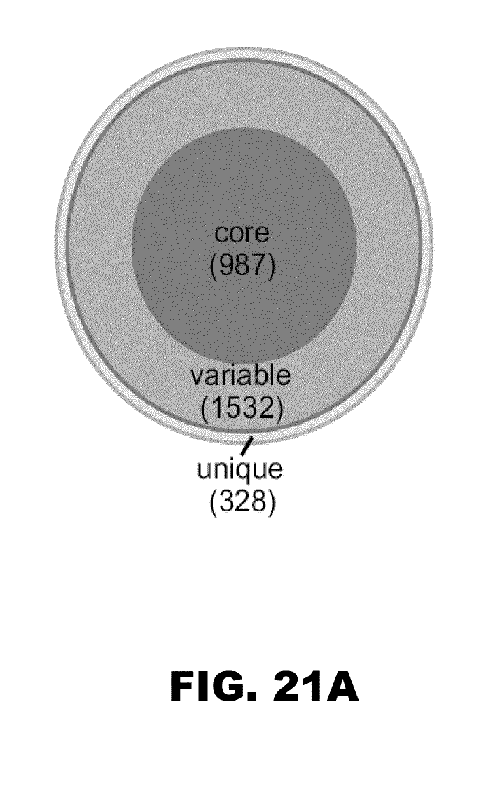

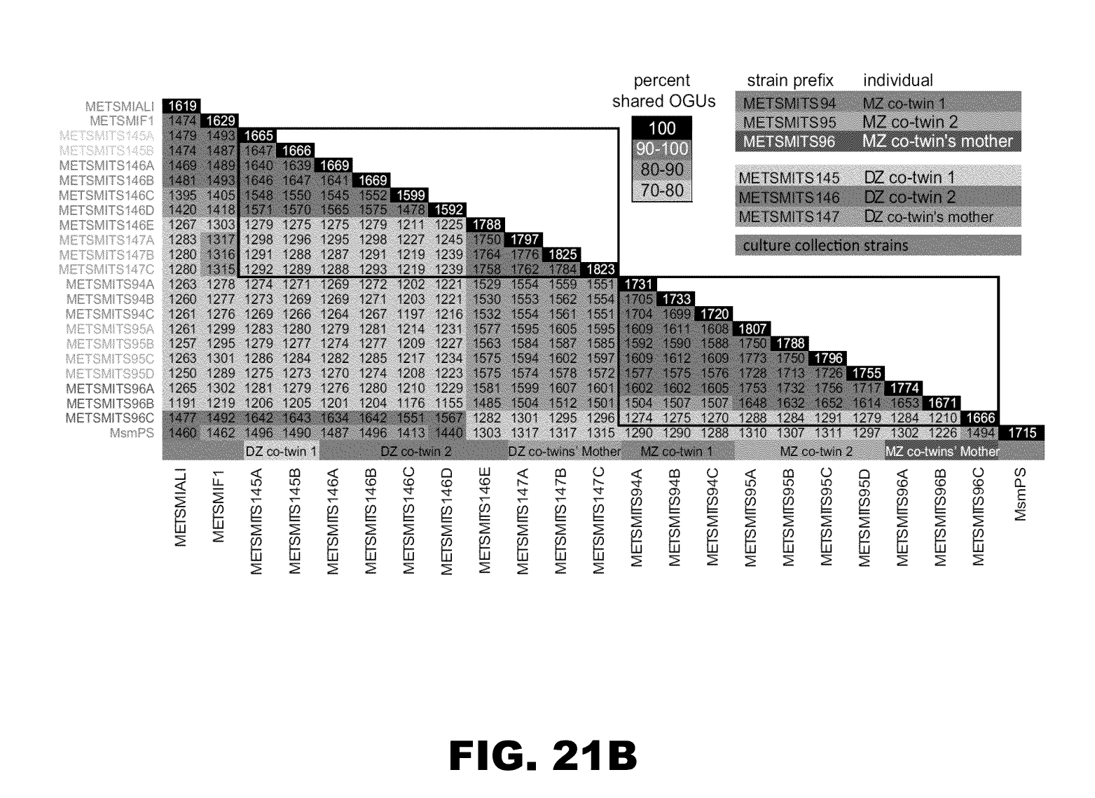

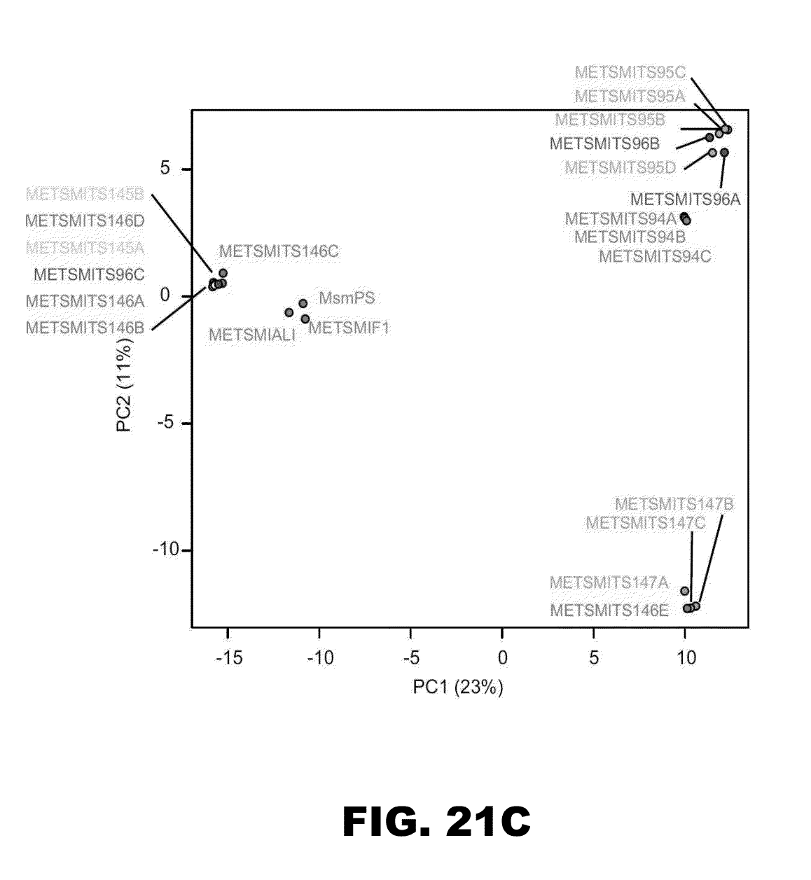

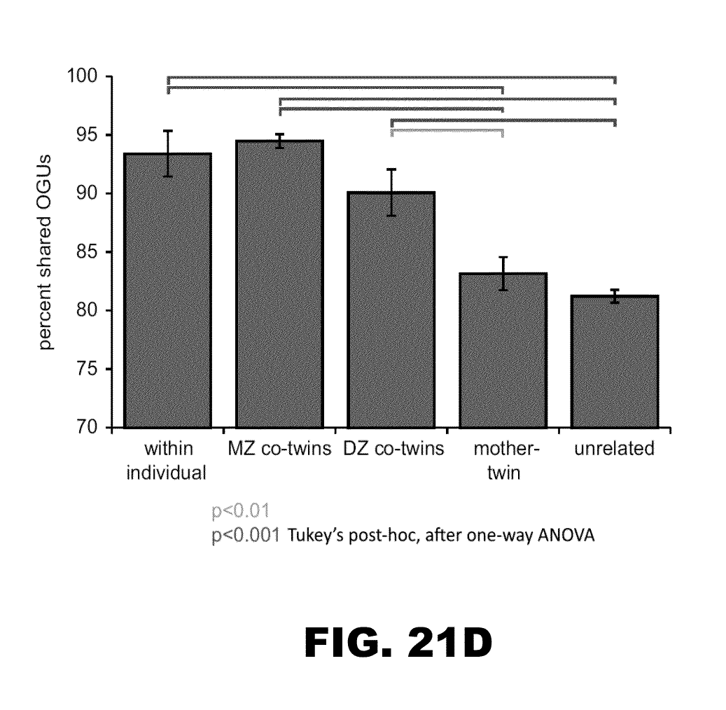

Genes were identified by using Glimmer (v3.02) trained on contigs >500 bp in each of the 20 sequenced M. smithii isolate genomes, plus the PS type strain and the two other M. smithii isolates we had sequenced. Genes in all 23 genomes were binned by using the program CD-HIT and its default parameters (>90% nucleotide sequence identity over of the length of the shorter gene in each pairwise comparison; FIG. 21) into “operational gene units” (OGUs), a term used in a way that is analogous to OTUs. If any predicted gene from an assembled genome was present in a given OGU bin, that OGU was called “present” within that genome (43). Functions were assigned to predicted proteins encoded by each gene by using the KEGG and STRING databases; Pfam and TIGRFAM annotations were also made. Note that all predicted protein-coding sequences <300 nt were filtered out and not considered in the analyses reported below.

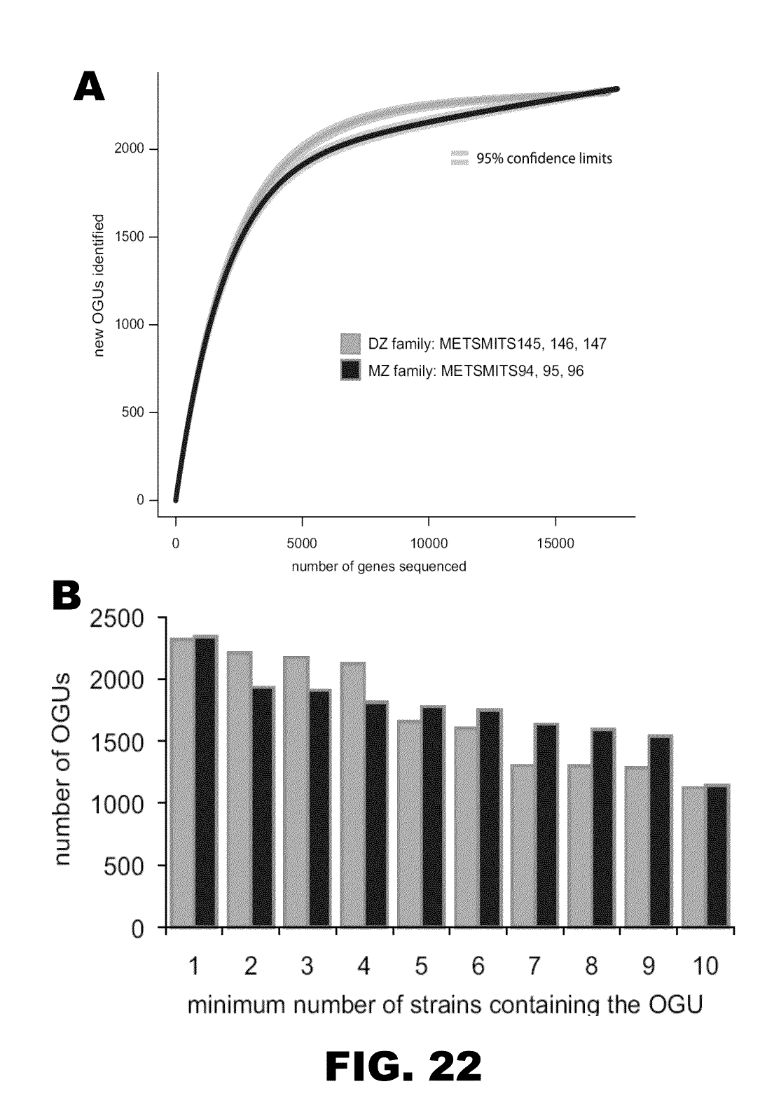

Rarefaction analysis to determine the rate at which sequencing the genes of new strains revealed new OGUs showed that the number of new or unique OGUs identified begins to plateau by the time≈6 strains were sequenced (≈10,000 genes) (FIGS. 22 A and B). A total of 987 OGUs were present in all 23 strains (34.7% of 2,847 identified OGUs), whereas 1,532 (53.8%) were found in more than one strain but not all, and 328 (11.5%) in only a single strain (FIGS. 21A and B).

PCA of OGU assignments showed clustering of strains based on family of origin: Strains from MZ family members (TS94-96) generally clustered together, whereas strains from the DZ family (TS145-147) split into two groups (FIG. 21C). Further pairwise comparisons of the degree of sharing of OGUs in strains showed that strains within an individual and within MZ and DZ co-twins shared significantly more OGUs than strains from the co-twin's mother or from unrelated individuals. Moreover, the degree of sharing of OGUs was not significantly different between MZ and DZ twin pairs (FIG. 21D). As noted above, MZ twins have greater concordance for carriage and levels of methanogens in their fecal microbiota than DZ twins. The fact that the sequenced strains are no more similar between MZ co-twins than DZ twins suggests that although shared environmental exposures to methanogens direct which strains are found in an individual's gut, long-term persistence is influenced by a combination of host and microbial genetic factors.

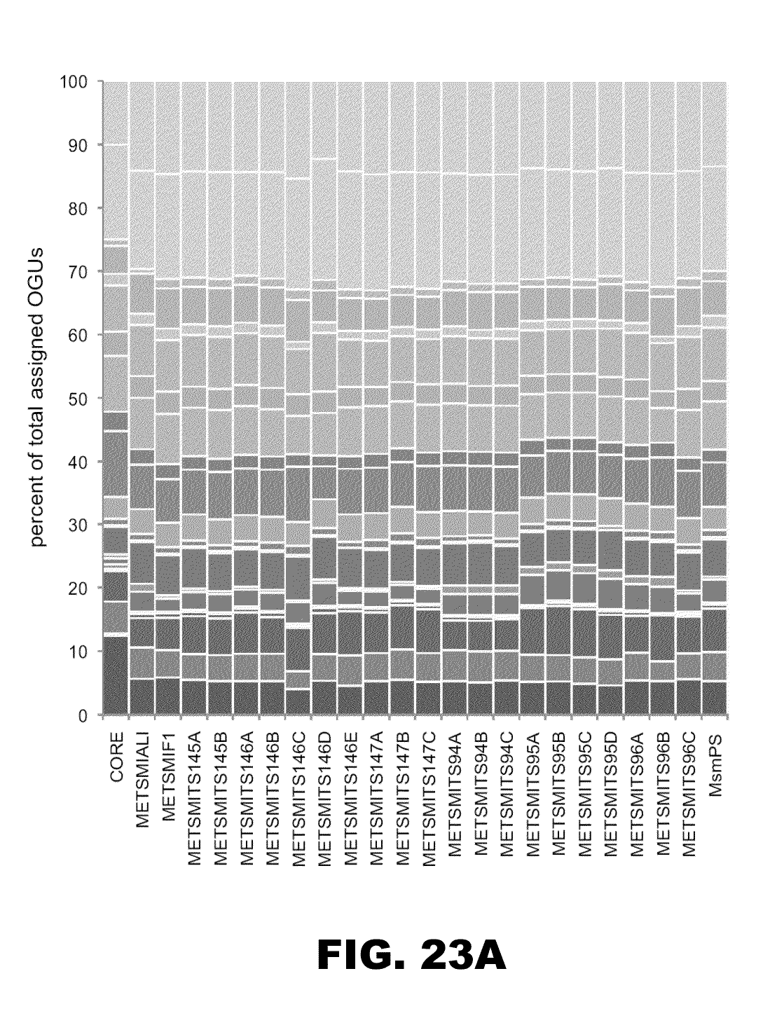

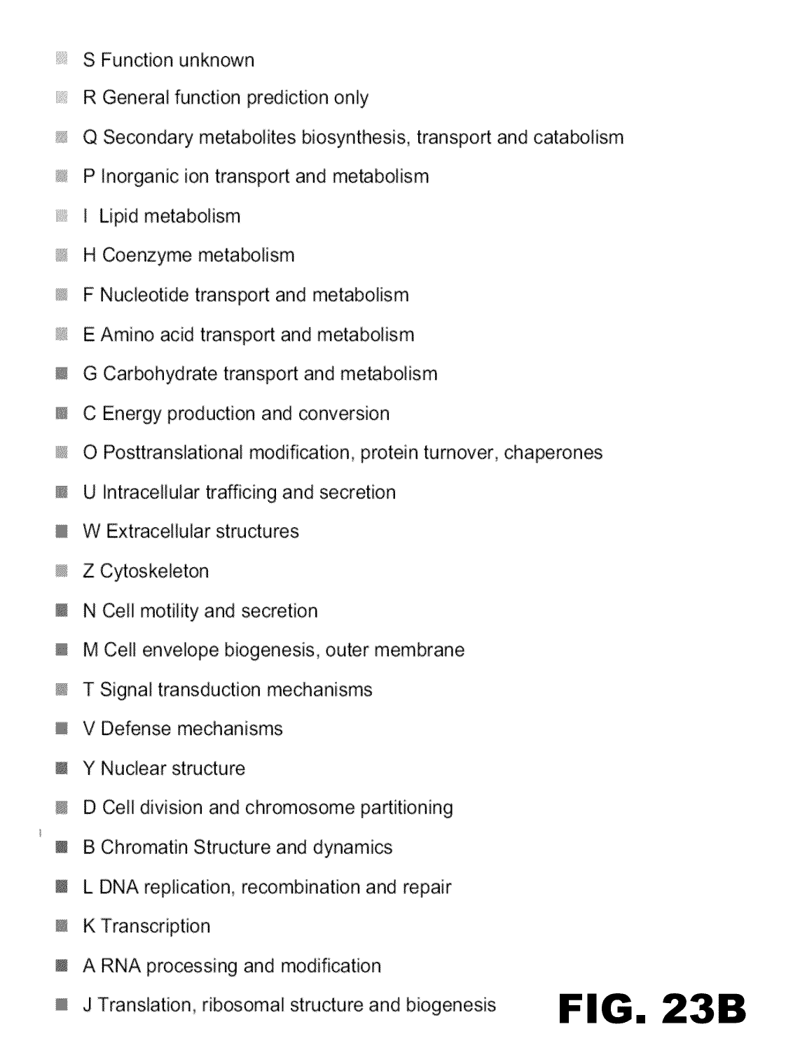

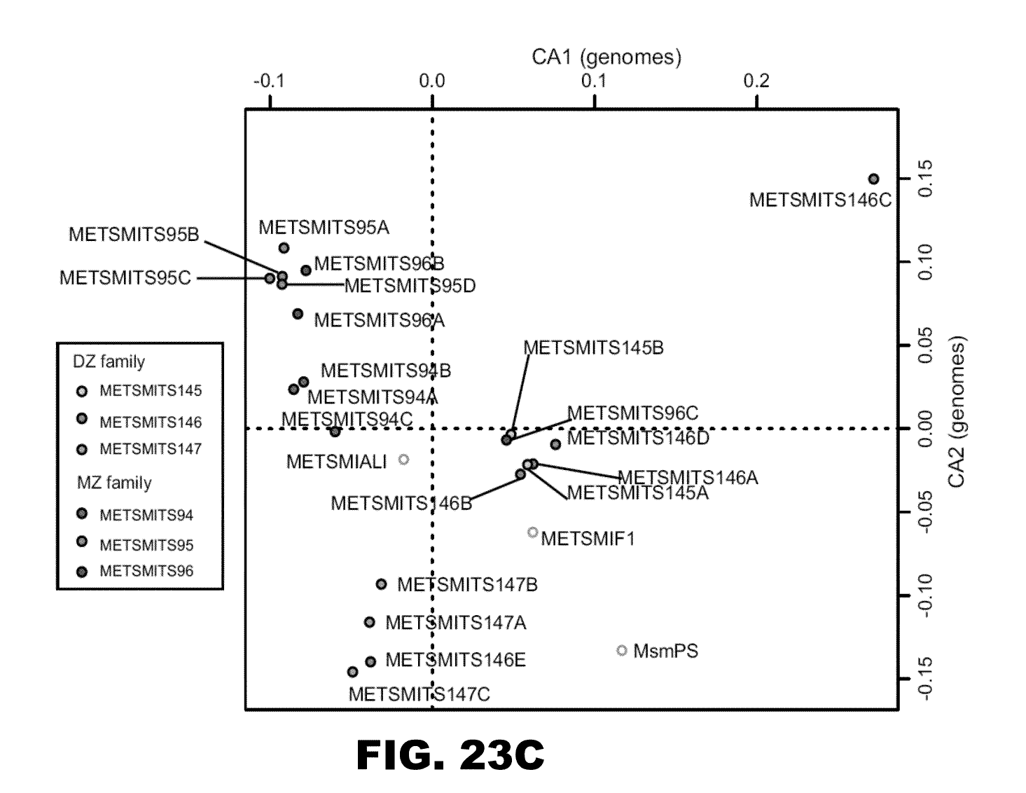

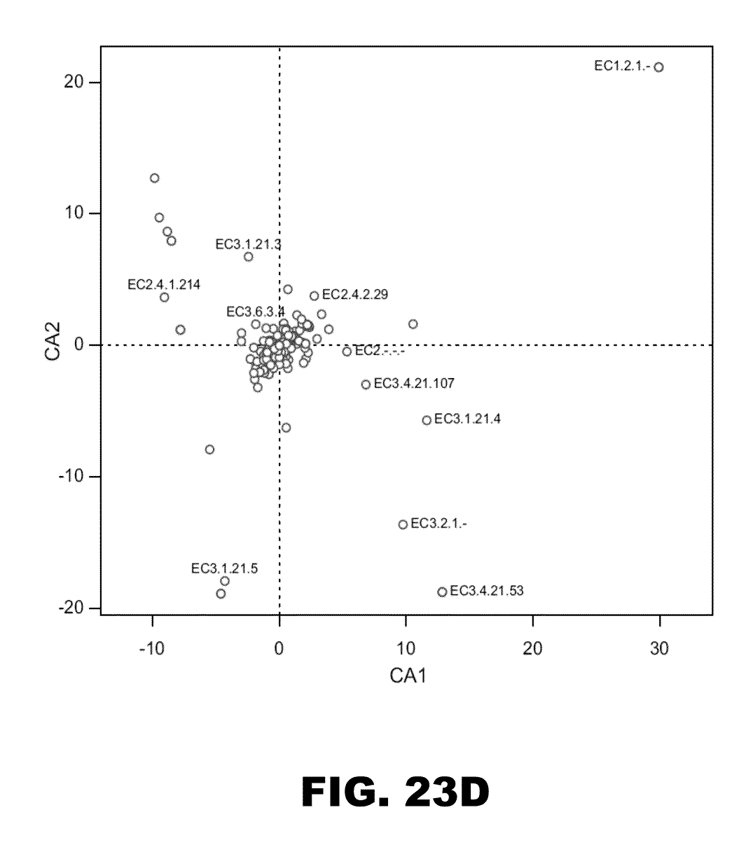

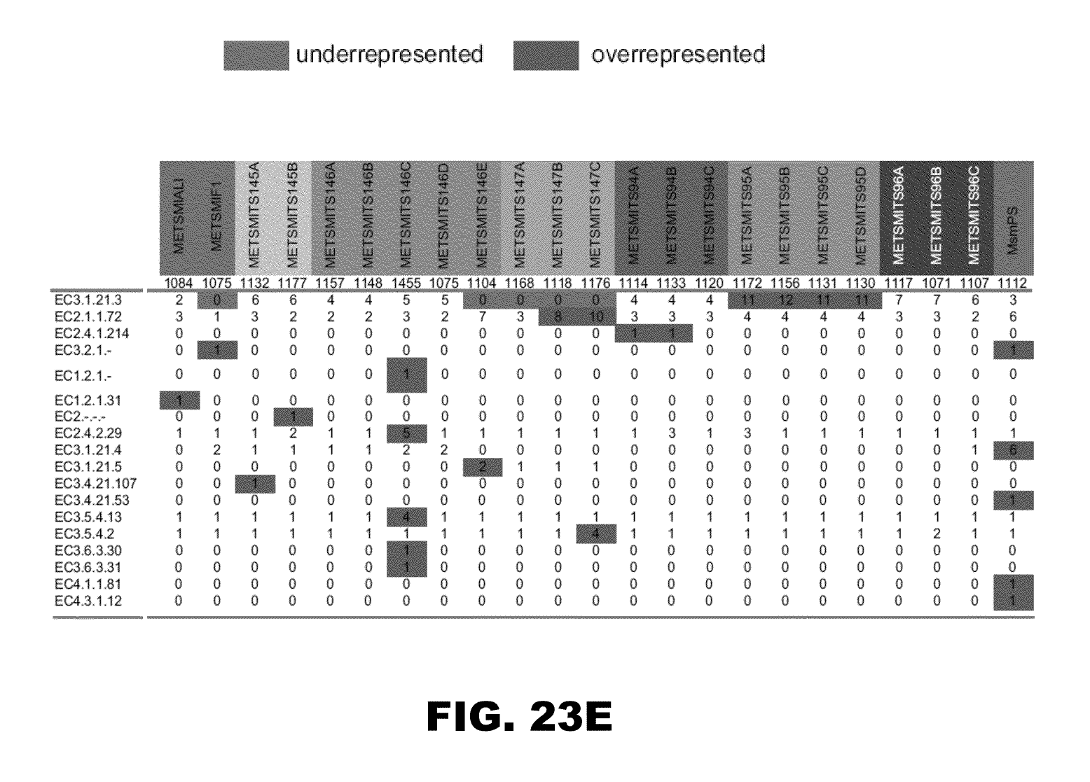

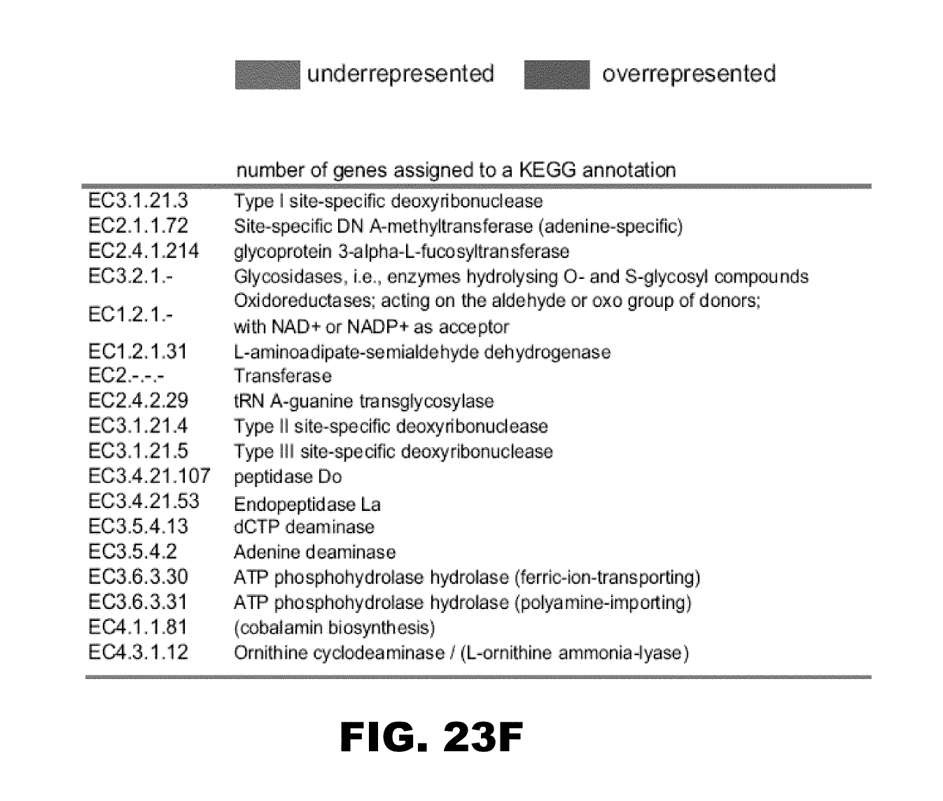

KEGG was used to assign enzyme commission (EC) numbers to genes in all of the isolates' genomes. A total of 412 ECs were identified: 349 were shared by all strains, 63 were variably represented, and 18 had significant differences in their representation between strains as judged by binomial test (FIG. 23D-E). These discriminatory ECs include (i) several restriction enzymes, (ii) two peptidases [a serine protease known as Do, HtrA, or DegP (44) that may protect against heat-stress and unfolded proteins and endopeptidase La (45)], both of which may be related to quality control in protein folding, and (iii) tRNA-guanine transglycosylases (involved in the anti-codon modification of tRNAs specific for Asn, Asp, His, and Tyr) (FIG. 23 B-E).

Genes assigned to COG M (cell envelope biogenesis/outer membrane) were prominently represented in the variable component of the pan-genome (FIG. 23A). Variability in surface proteins may directly impact the fitness of M. smithii strains in vivo, including their ability to adhere to host structures, or to interact with syntrophic partners. For example, all of the M. smithii strains contain the six genes involved in synthesis of pseudaminic acid structures related to sialic acid molecules expressed on host cell surfaces. The resulting surface epitopes are thought to play a role in the adaptation of M. smithii to the gut environment by mimicking the sialic acids that decorate the surfaces of host epithelial cells (46). Adhesin-like proteins (ALPs) are a novel class of proteins with homology to bacterial adhesins that were first identified in the M. smithii type strain. They are also hypothesized to play a role in adaptation to the gut environment (42). The 23 sequenced strains contain a total of 101 ALP OGUs (average 45±6 ALP genes per strain). Only six were present in all strains. ALP sequences are quite divergent in terms of their domain structure: e.g., many have intimin domains, which in Escherichia coli mediate binding to intestinal epithelial cells; others have pectate lyase domains and/or parallel β-helix repeats that are often found in enzymes with polysaccharide substrates. Tables 33 and B-D summarize the ALP data.

To better understand genomic differences among M. smithii strains, the M. smithii pan-genome was searched for evidence of horizontal gene transfer (HGT). The results, described below in Example 11 and summarized in Table 27, show that HGT has contributed to both the core and variable elements of the M. smithii pan-genome. They include core genes involved in methanogenesis and folate biosynthesis; e.g., both compositional- and phylogenetic-based methods revealed transfer of genes encoding THMP methyltransferase C subunit (EC 2.1.1.86), formate dehydrogenase (EC 1.2.1.2), and formylmethanofuran dehydrogenase subunit F (E.C. 1.2.99.5) (Table 28). Note that the early steps in synthesis of methanopterin, a C1 carrier coenzyme involved in the methanogenesis pathway (FIG. 24), are the same as those used for generation of folate (Table 28). In addition, between 52% and 65% ALPs show evidence of transfer: Large-scale HGT of ALPs would be consistent with their variability among strains (Table 20).

TABLE 27
Distribution of HGT genes in the core, variable
and pan-genome by detection method.

| | Variable Genome | | Core Genome | |
|---|---|---|---|---|
| Category* | Genes | % | Genes | % |
| Codons | 2695 | 67.8% | 1278 | 32.2% |
| Codons (with KO mappings) | 816 | 46.6% | 935 | 53.4% |
| Dinuc 3-1 | 2858 | 68.0% | 1342 | 32.0% |
| Dinuc 3-1 (with KO mappings) | 756 | 42.5% | 1023 | 57.5% |
| K-words order 5 | 1386 | 59.3% | 950 | 40.7% |
| K-words order 5 (with KO | 418 | 32.2% | 879 | 67.8% |
| mappings) | | | | |
| PhyloNet | 1333 | 26.0% | 3790 | 73.4% |
| PhyloNet and codons | 174 | 54.5% | 145 | 45.5% |
| PhyloNet and dinuc 3-1 | 146 | 45.9% | 172 | 54.1% |
| PhyloNet and kwords order 5 | 114 | 40.7% | 166 | 59.3% |
| Phage | 17 | 10.9% | 139 | 89.1% |

*Categories listed as ‘with KO mappings’ represent the subset of the pan-genome that could be mapped to KEGG orthology groups.

TABLE 28
Genes involved in methane metabolism and folate biosynthesis that show evidence of HGT

| Analyses used | GENE_ID | KO_ID |
|---|---|---|
| PhyloNet and codon_usage_G_score_rank_order_threshold | METSMIALI_0037 | K00205 |
| PhyloNet and codon_usage_G_score_rank_order_threshold | METSMIALI_0955 | K00205 |
| PhyloNet and codon_usage_G_score_rank_order_threshold | METSMIF1_0715 | K00205 |
| PhyloNet and codon_usage_G_score_rank_order_threshold | METSMIF1_1646 | K00205 |
| PhyloNet and codon_usage_G_score_rank_order_threshold | METSMITS145A_0445 | K00205 |
| PhyloNet and codon_usage_G_score_rank_order_threshold | METSMITS145A_1154 | K00205 |
| PhyloNet and codon_usage_G_score_rank_order_threshold | METSMITS145A_1594 | K00205 |
| PhyloNet and codon_usage_G_score_rank_order_threshold | METSMITS145B_0331 | K00205 |
| PhyloNet and codon_usage_G_score_rank_order_threshold | METSMITS145B_0824 | K00205 |
| PhyloNet and codon_usage_G_score_rank_order_threshold | METSMITS145B_1389 | K00205 |
| PhyloNet and codon_usage_G_score_rank_order_threshold | METSMITS146A_0513 | K00205 |
| PhyloNet and codon_usage_G_score_rank_order_threshold | METSMITS146A_0828 | K00205 |
| PhyloNet and codon_usage_G_score_rank_order_threshold | METSMITS146A_1220 | K00205 |
| PhyloNet and codon_usage_G_score_rank_order_threshold | METSMITS146B_0324 | K00205 |
| PhyloNet and codon_usage_G_score_rank_order_threshold | METSMITS146B_0819 | K00205 |
| PhyloNet and codon_usage_G_score_rank_order_threshold | METSMITS146B_1209 | K00205 |
| PhyloNet and codon_usage_G_score_rank_order_threshold | METSMITS146C_0709 | K00205 |
| PhyloNet and codon_usage_G_score_rank_order_threshold | METSMITS146C_1260 | K00205 |
| PhyloNet and codon_usage_G_score_rank_order_threshold | METSMITS146D_0301 | K08264 |
| PhyloNet and codon_usage_G_score_rank_order_threshold | METSMITS146D_0713 | K00205 |
| PhyloNet and codon_usage_G_score_rank_order_threshold | METSMITS146D_1094 | K00205 |
| PhyloNet and codon_usage_G_score_rank_order_threshold | METSMITS146E_0322 | K00205 |
| PhyloNet and codon_usage_G_score_rank_order_threshold | METSMITS146E_1157 | K00205 |
| PhyloNet and codon_usage_G_score_rank_order_threshold | METSMITS146E_1543 | K00205 |
| PhyloNet and codon_usage_G_score_rank_order_threshold | METSMITS147A_0308 | K00205 |
| PhyloNet and codon_usage_G_score_rank_order_threshold | METSMITS147A_1146 | K00205 |
| PhyloNet and codon_usage_G_score_rank_order_threshold | METSMITS147A_1579 | K00205 |
| PhyloNet and codon_usage_G_score_rank_order_threshold | METSMITS147B_0324 | K00205 |
| PhyloNet and codon_usage_G_score_rank_order_threshold | METSMITS147B_1201 | K00205 |
| PhyloNet and codon_usage_G_score_rank_order_threshold | METSMITS147B_1635 | K00205 |
| PhyloNet and codon_usage_G_score_rank_order_threshold | METSMITS147C_0335 | K00205 |
| PhyloNet and codon_usage_G_score_rank_order_threshold | METSMITS147C_1084 | K00205 |
| PhyloNet and codon_usage_G_score_rank_order_threshold | METSMITS147C_1668 | K00205 |
| PhyloNet and codon_usage_G_score_rank_order_threshold | METSMITS94A_0260 | K00205 |
| PhyloNet and codon_usage_G_score_rank_order_threshold | METSMITS94A_1080 | K00205 |
| PhyloNet and codon_usage_G_score_rank_order_threshold | METSMITS94B_0260 | K00205 |
| PhyloNet and codon_usage_G_score_rank_order_threshold | METSMITS94B_1083 | K00205 |
| PhyloNet and codon_usage_G_score_rank_order_threshold | METSMITS94B_1486 | K00205 |
| PhyloNet and codon_usage_G_score_rank_order_threshold | METSMITS94C_0268 | K00205 |
| PhyloNet and codon_usage_G_score_rank_order_threshold | METSMITS94C_1067 | K00205 |
| PhyloNet and codon_usage_G_score_rank_order_threshold | METSMITS94C_1473 | K00205 |
| PhyloNet and codon_usage_G_score_rank_order_threshold | METSMITS95A_0364 | K00205 |
| PhyloNet and codon_usage_G_score_rank_order_threshold | METSMITS95A_1143 | K00205 |
| PhyloNet and codon_usage_G_score_rank_order_threshold | METSMITS95A_1614 | K00205 |
| PhyloNet and codon_usage_G_score_rank_order_threshold | METSMITS95B_0355 | K00205 |
| PhyloNet and codon_usage_G_score_rank_order_threshold | METSMITS95B_0439 | K08264 |
| PhyloNet and codon_usage_G_score_rank_order_threshold | METSMITS95B_1120 | K00205 |
| PhyloNet and codon_usage_G_score_rank_order_threshold | METSMITS95C_0410 | K00205 |
| PhyloNet and codon_usage_G_score_rank_order_threshold | METSMITS95C_1166 | K00205 |
| PhyloNet and codon_usage_G_score_rank_order_threshold | METSMITS95C_1610 | K00205 |
| PhyloNet and codon_usage_G_score_rank_order_threshold | METSMITS95D_0359 | K00205 |
| PhyloNet and codon_usage_G_score_rank_order_threshold | METSMITS95D_1075 | K00205 |
| PhyloNet and codon_usage_G_score_rank_order_threshold | METSMITS95D_1511 | K00205 |
| PhyloNet and codon_usage_G_score_rank_order_threshold | METSMITS96A_0361 | K00205 |
| PhyloNet and codon_usage_G_score_rank_order_threshold | METSMITS96A_1105 | K00205 |
| PhyloNet and codon_usage_G_score_rank_order_threshold | METSMITS96A_1557 | K00205 |
| PhyloNet and codon_usage_G_score_rank_order_threshold | METSMITS96B_0381 | K00205 |
| PhyloNet and codon_usage_G_score_rank_order_threshold | METSMITS96B_0450 | K08264 |
| PhyloNet and codon_usage_G_score_rank_order_threshold | METSMITS96B_1035 | K00205 |
| PhyloNet and codon_usage_G_score_rank_order_threshold | METSMITS96B_1401 | K00205 |
| PhyloNet and codon_usage_G_score_rank_order_threshold | METSMITS96C_0321 | K00205 |
| PhyloNet and codon_usage_G_score_rank_order_threshold | METSMITS96C_1067 | K00205 |
| PhyloNet and codon_usage_G_score_rank_order_threshold | METSMITS96C_1390 | K00205 |
| PhyloNet and dinuc_3_1_G_score_rank_order_threshold | METSMIALI_0886 | K00205 |
| PhyloNet and dinuc_3_1_G_score_rank_order_threshold | METSMIF1_0380 | K00205 |
| PhyloNet and dinuc_3_1_G_score_rank_order_threshold | METSMIF1_0784 | K00205 |
| PhyloNet and dinuc_3_1_G_score_rank_order_threshold | METSMITS145A_0864 | K00205 |
| PhyloNet and dinuc_3_1_G_score_rank_order_threshold | METSMITS145A_1086 | K00205 |
| PhyloNet and dinuc_3_1_G_score_rank_order_threshold | METSMITS145B_0772 | K00205 |
| PhyloNet and dinuc_3_1_G_score_rank_order_threshold | METSMITS145B_1316 | K00205 |
| PhyloNet and dinuc_3_1_G_score_rank_order_threshold | METSMITS146A_0746 | K00205 |
| PhyloNet and dinuc_3_1_G_score_rank_order_threshold | METSMITS146A_1149 | K00205 |
| PhyloNet and dinuc_3_1_G_score_rank_order_threshold | METSMITS146B_0749 | K00205 |
| PhyloNet and dinuc_3_1_G_score_rank_order_threshold | METSMITS146B_1139 | K00205 |
| PhyloNet and dinuc_3_1_G_score_rank_order_threshold | METSMITS146C_1151 | K00205 |
| PhyloNet and dinuc_3_1_G_score_rank_order_threshold | METSMITS146D_0647 | K00205 |
| PhyloNet and dinuc_3_1_G_score_rank_order_threshold | METSMITS146D_1022 | K00205 |
| PhyloNet and dinuc_3_1_G_score_rank_order_threshold | METSMITS146E_1088 | K00205 |
| PhyloNet and dinuc_3_1_G_score_rank_order_threshold | METSMITS147A_1078 | K00205 |
| PhyloNet and dinuc_3_1_G_score_rank_order_threshold | METSMITS147B_1133 | K00205 |
| PhyloNet and dinuc_3_1_G_score_rank_order_threshold | METSMITS147C_0650 | K00320 |
| PhyloNet and dinuc_3_1_G_score_rank_order_threshold | METSMITS147C_1016 | K00205 |
| PhyloNet and dinuc_3_1_G_score_rank_order_threshold | METSMITS94A_1012 | K00205 |
| PhyloNet and dinuc_3_1_G_score_rank_order_threshold | METSMITS94B_1008 | K00205 |
| PhyloNet and dinuc_3_1_G_score_rank_order_threshold | METSMITS94C_0517 | K00320 |
| PhyloNet and dinuc_3_1_G_score_rank_order_threshold | METSMITS94C_0999 | K00205 |
| PhyloNet and dinuc_3_1_G_score_rank_order_threshold | METSMITS95A_0601 | K00320 |
| PhyloNet and dinuc_3_1_G_score_rank_order_threshold | METSMITS95A_1071 | K00205 |
| PhyloNet and dinuc_3_1_G_score_rank_order_threshold | METSMITS95B_0589 | K00320 |
| PhyloNet and dinuc_3_1_G_score_rank_order_threshold | METSMITS95B_1051 | K00205 |
| PhyloNet and dinuc_3_1_G_score_rank_order_threshold | METSMITS95C_0643 | K00320 |
| PhyloNet and dinuc_3_1_G_score_rank_order_threshold | METSMITS95C_1099 | K00205 |
| PhyloNet and dinuc_3_1_G_score_rank_order_threshold | METSMITS95D_1007 | K00205 |
| PhyloNet and dinuc_3_1_G_score_rank_order_threshold | METSMITS96A_1037 | K00205 |
| PhyloNet and dinuc_3_1_G_score_rank_order_threshold | METSMITS96B_0594 | K00320 |
| PhyloNet and dinuc_3_1_G_score_rank_order_threshold | METSMITS96B_0967 | K00205 |
| PhyloNet and dinuc_3_1_G_score_rank_order_threshold | METSMITS96C_0997 | K00205 |
| PhyloNet and dinuc_3_1_G_score_rank_order_threshold | METSMITS96C_1735 | K00205 |
| PhyloNet and kwords_order_2_G_score_rank_order_threshold | METSMIALI_1289 | K00122 |
| PhyloNet and kwords_order_2_G_score_rank_order_threshold | METSMIF1_0386 | K00122 |
| PhyloNet and kwords_order_2_G_score_rank_order_threshold | METSMITS145A_0870 | K00122 |
| PhyloNet and kwords_order_2_G_score_rank_order_threshold | METSMITS145B_0778 | K00122 |
| PhyloNet and kwords_order_2_G_score_rank_order_threshold | METSMITS146A_0752 | K00122 |
| PhyloNet and kwords_order_2_G_score_rank_order_threshold | METSMITS146B_0755 | K00122 |
| PhyloNet and kwords_order_2_G_score_rank_order_threshold | METSMITS146C_1672 | K00122 |
| PhyloNet and kwords_order_2_G_score_rank_order_threshold | METSMITS146D_0653 | K00122 |
| PhyloNet and kwords_order_2_G_score_rank_order_threshold | METSMITS146E_0783 | K00122 |
| PhyloNet and kwords_order_2_G_score_rank_order_threshold | METSMITS147A_0742 | K00122 |
| PhyloNet and kwords_order_2_G_score_rank_order_threshold | METSMITS147B_1564 | K00122 |
| PhyloNet and kwords_order_2_G_score_rank_order_threshold | METSMITS147C_0851 | K00122 |
| PhyloNet and kwords_order_2_G_score_rank_order_threshold | METSMITS94A_0708 | K00122 |
| PhyloNet and kwords_order_2_G_score_rank_order_threshold | METSMITS94B_0714 | K00122 |
| PhyloNet and kwords_order_2_G_score_rank_order_threshold | METSMITS94C_0711 | K00122 |
| PhyloNet and kwords_order_2_G_score_rank_order_threshold | METSMITS95A_1453 | K00122 |
| PhyloNet and kwords_order_2_G_score_rank_order_threshold | METSMITS95B_1495 | K00122 |
| PhyloNet and kwords_order_2_G_score_rank_order_threshold | METSMITS95C_1536 | K00122 |
| PhyloNet and kwords_order_2_G_score_rank_order_threshold | METSMITS95D_1442 | K00122 |
| PhyloNet and kwords_order_2_G_score_rank_order_threshold | METSMITS96A_1475 | K00122 |
| PhyloNet and kwords_order_2_G_score_rank_order_threshold | METSMITS96B_1323 | K00122 |
| PhyloNet and kwords_order_2_G_score_rank_order_threshold | METSMITS96C_1729 | K00122 |
| PhyloNet and kwords_order_2_G_score_rank_order_threshold | METSMIALI_0037 | K00205 |
| PhyloNet and kwords_order_2_G_score_rank_order_threshold | METSMIALI_0886 | K00205 |
| PhyloNet and kwords_order_2_G_score_rank_order_threshold | METSMIF1_0784 | K00205 |
| PhyloNet and kwords_order_2_G_score_rank_order_threshold | METSMIF1_1646 | K00205 |
| PhyloNet and kwords_order_2_G_score_rank_order_threshold | METSMITS145A_0445 | K00205 |
| PhyloNet and kwords_order_2_G_score_rank_order_threshold | METSMITS145A_1086 | K00205 |
| PhyloNet and kwords_order_2_G_score_rank_order_threshold | METSMITS145A_1594 | K00205 |
| PhyloNet and kwords_order_2_G_score_rank_order_threshold | METSMITS145B_0331 | K00205 |
| PhyloNet and kwords_order_2_G_score_rank_order_threshold | METSMITS145B_0772 | K00205 |
| PhyloNet and kwords_order_2_G_score_rank_order_threshold | METSMITS145B_0824 | K00205 |
| PhyloNet and kwords_order_2_G_score_rank_order_threshold | METSMITS145B_1316 | K00205 |
| PhyloNet and kwords_order_2_G_score_rank_order_threshold | METSMITS146A_0513 | K00205 |
| PhyloNet and kwords_order_2_G_score_rank_order_threshold | METSMITS146A_0746 | K00205 |
| PhyloNet and kwords_order_2_G_score_rank_order_threshold | METSMITS146A_0828 | K00205 |
| PhyloNet and kwords_order_2_G_score_rank_order_threshold | METSMITS146A_1149 | K00205 |
| PhyloNet and kwords_order_2_G_score_rank_order_threshold | METSMITS146B_0324 | K00205 |
| PhyloNet and kwords_order_2_G_score_rank_order_threshold | METSMITS146B_0749 | K00205 |
| PhyloNet and kwords_order_2_G_score_rank_order_threshold | METSMITS146B_0819 | K00205 |
| PhyloNet and kwords_order_2_G_score_rank_order_threshold | METSMITS146B_1139 | K00205 |
| PhyloNet and kwords_order_2_G_score_rank_order_threshold | METSMITS146C_0709 | K00205 |
| PhyloNet and kwords_order_2_G_score_rank_order_threshold | METSMITS146C_1151 | K00205 |
| PhyloNet and kwords_order_2_G_score_rank_order_threshold | METSMITS146C_1680 | K00205 |
| PhyloNet and kwords_order_2_G_score_rank_order_threshold | METSMITS146D_0647 | K00205 |
| PhyloNet and kwords_order_2_G_score_rank_order_threshold | METSMITS146D_1022 | K00205 |
| PhyloNet and kwords_order_2_G_score_rank_order_threshold | METSMITS146E_0322 | K00205 |
| PhyloNet and kwords_order_2_G_score_rank_order_threshold | METSMITS146E_1088 | K00205 |
| PhyloNet and kwords_order_2_G_score_rank_order_threshold | METSMITS146E_1102 | K00579 |
| PhyloNet and kwords_order_2_G_score_rank_order_threshold | METSMITS146E_1543 | K00205 |
| PhyloNet and kwords_order_2_G_score_rank_order_threshold | METSMITS147A_0308 | K00205 |
| PhyloNet and kwords_order_2_G_score_rank_order_threshold | METSMITS147A_1078 | K00205 |
| PhyloNet and kwords_order_2_G_score_rank_order_threshold | METSMITS147A_1092 | K00579 |
| PhyloNet and kwords_order_2_G_score_rank_order_threshold | METSMITS147A_1579 | K00205 |
| PhyloNet and kwords_order_2_G_score_rank_order_threshold | METSMITS147B_0324 | K00205 |
| PhyloNet and kwords_order_2_G_score_rank_order_threshold | METSMITS147B_1133 | K00205 |
| PhyloNet and kwords_order_2_G_score_rank_order_threshold | METSMITS147B_1147 | K00579 |
| PhyloNet and kwords_order_2_G_score_rank_order_threshold | METSMITS147B_1635 | K00205 |
| PhyloNet and kwords_order_2_G_score_rank_order_threshold | METSMITS147C_0335 | K00205 |
| PhyloNet and kwords_order_2_G_score_rank_order_threshold | METSMITS147C_1016 | K00205 |
| PhyloNet and kwords_order_2_G_score_rank_order_threshold | METSMITS147C_1030 | K00579 |
| PhyloNet and kwords_order_2_G_score_rank_order_threshold | METSMITS147C_1668 | K00205 |
| PhyloNet and kwords_order_2_G_score_rank_order_threshold | METSMITS94A_1012 | K00205 |
| PhyloNet and kwords_order_2_G_score_rank_order_threshold | METSMITS94A_1026 | K00579 |
| PhyloNet and kwords_order_2_G_score_rank_order_threshold | METSMITS94B_1008 | K00205 |
| PhyloNet and kwords_order_2_G_score_rank_order_threshold | METSMITS94B_1023 | K00579 |
| PhyloNet and kwords_order_2_G_score_rank_order_threshold | METSMITS94B_1486 | K00205 |
| PhyloNet and kwords_order_2_G_score_rank_order_threshold | METSMITS94C_0999 | K00205 |
| PhyloNet and kwords_order_2_G_score_rank_order_threshold | METSMITS94C_1013 | K00579 |
| PhyloNet and kwords_order_2_G_score_rank_order_threshold | METSMITS94C_1473 | K00205 |
| PhyloNet and kwords_order_2_G_score_rank_order_threshold | METSMITS95A_1071 | K00205 |
| PhyloNet and kwords_order_2_G_score_rank_order_threshold | METSMITS95A_1087 | K00579 |
| PhyloNet and kwords_order_2_G_score_rank_order_threshold | METSMITS95A_1614 | K00205 |
| PhyloNet and kwords_order_2_G_score_rank_order_threshold | METSMITS95B_1051 | K00205 |
| PhyloNet and kwords_order_2_G_score_rank_order_threshold | METSMITS95B_1065 | K00579 |
| PhyloNet and kwords_order_2_G_score_rank_order_threshold | METSMITS95C_1099 | K00205 |
| PhyloNet and kwords_order_2_G_score_rank_order_threshold | METSMITS95C_1112 | K00579 |
| PhyloNet and kwords_order_2_G_score_rank_order_threshold | METSMITS95C_1610 | K00205 |
| PhyloNet and kwords_order_2_G_score_rank_order_threshold | METSMITS95D_1007 | K00205 |
| PhyloNet and kwords_order_2_G_score_rank_order_threshold | METSMITS95D_1021 | K00579 |
| PhyloNet and kwords_order_2_G_score_rank_order_threshold | METSMITS95D_1511 | K00205 |
| PhyloNet and kwords_order_2_G_score_rank_order_threshold | METSMITS96A_1037 | K00205 |
| PhyloNet and kwords_order_2_G_score_rank_order_threshold | METSMITS96A_1051 | K00579 |
| PhyloNet and kwords_order_2_G_score_rank_order_threshold | METSMITS96A_1557 | K00205 |
| PhyloNet and kwords_order_2_G_score_rank_order_threshold | METSMITS96B_0967 | K00205 |
| PhyloNet and kwords_order_2_G_score_rank_order_threshold | METSMITS96B_0981 | K00579 |
| PhyloNet and kwords_order_2_G_score_rank_order_threshold | METSMITS96B_1401 | K00205 |
| PhyloNet and kwords_order_2_G_score_rank_order_threshold | METSMITS96C_0321 | K00205 |
| PhyloNet and kwords_order_2_G_score_rank_order_threshold | METSMITS96C_0997 | K00205 |
| PhyloNet and kwords_order_2_G_score_rank_order_threshold | METSMITS96C_1390 | K00205 |
| PhyloNet and kwords_order_2_G_score_rank_order_threshold | METSMITS96C_1735 | K00205 |
| PhyloNet and kwords_order_3_G_score_rank_order_threshold | METSMIALI_1289 | K00122 |
| PhyloNet and kwords_order_3_G_score_rank_order_threshold | METSMIF1_0386 | K00122 |
| PhyloNet and kwords_order_3_G_score_rank_order_threshold | METSMITS145A_0870 | K00122 |
| PhyloNet and kwords_order_3_G_score_rank_order_threshold | METSMITS145B_0778 | K00122 |
| PhyloNet and kwords_order_3_G_score_rank_order_threshold | METSMITS146A_0752 | K00122 |
| PhyloNet and kwords_order_3_G_score_rank_order_threshold | METSMITS146B_0755 | K00122 |
| PhyloNet and kwords_order_3_G_score_rank_order_threshold | METSMITS146C_1672 | K00122 |
| PhyloNet and kwords_order_3_G_score_rank_order_threshold | METSMITS146D_0653 | K00122 |
| PhyloNet and kwords_order_3_G_score_rank_order_threshold | METSMITS146E_0783 | K00122 |
| PhyloNet and kwords_order_3_G_score_rank_order_threshold | METSMITS147A_0742 | K00122 |
| PhyloNet and kwords_order_3_G_score_rank_order_threshold | METSMITS147B_1564 | K00122 |
| PhyloNet and kwords_order_3_G_score_rank_order_threshold | METSMITS147C_0851 | K00122 |
| PhyloNet and kwords_order_3_G_score_rank_order_threshold | METSMITS94A_0708 | K00122 |
| PhyloNet and kwords_order_3_G_score_rank_order_threshold | METSMITS94B_0714 | K00122 |
| PhyloNet and kwords_order_3_G_score_rank_order_threshold | METSMITS94C_0711 | K00122 |
| PhyloNet and kwords_order_3_G_score_rank_order_threshold | METSMITS95A_1453 | K00122 |
| PhyloNet and kwords_order_3_G_score_rank_order_threshold | METSMITS95B_1495 | K00122 |
| PhyloNet and kwords_order_3_G_score_rank_order_threshold | METSMITS95C_1536 | K00122 |
| PhyloNet and kwords_order_3_G_score_rank_order_threshold | METSMITS95D_1442 | K00122 |
| PhyloNet and kwords_order_3_G_score_rank_order_threshold | METSMITS96A_1475 | K00122 |
| PhyloNet and kwords_order_3_G_score_rank_order_threshold | METSMITS96B_1323 | K00122 |
| PhyloNet and kwords_order_3_G_score_rank_order_threshold | METSMITS96C_1729 | K00122 |
| PhyloNet and kwords_order_3_G_score_rank_order_threshold | METSMIALI_0037 | K00205 |
| PhyloNet and kwords_order_3_G_score_rank_order_threshold | METSMIALI_0886 | K00205 |
| PhyloNet and kwords_order_3_G_score_rank_order_threshold | METSMIF1_0784 | K00205 |
| PhyloNet and kwords_order_3_G_score_rank_order_threshold | METSMIF1_1646 | K00205 |
| PhyloNet and kwords_order_3_G_score_rank_order_threshold | METSMITS145A_0445 | K00205 |
| PhyloNet and kwords_order_3_G_score_rank_order_threshold | METSMITS145A_1086 | K00205 |
| PhyloNet and kwords_order_3_G_score_rank_order_threshold | METSMITS145A_1594 | K00205 |
| PhyloNet and kwords_order_3_G_score_rank_order_threshold | METSMITS145B_0331 | K00205 |
| PhyloNet and kwords_order_3_G_score_rank_order_threshold | METSMITS145B_0824 | K00205 |
| PhyloNet and kwords_order_3_G_score_rank_order_threshold | METSMITS145B_1316 | K00205 |
| PhyloNet and kwords_order_3_G_score_rank_order_threshold | METSMITS146A_0513 | K00205 |
| PhyloNet and kwords_order_3_G_score_rank_order_threshold | METSMITS146A_0828 | K00205 |
| PhyloNet and kwords_order_3_G_score_rank_order_threshold | METSMITS146A_1149 | K00205 |
| PhyloNet and kwords_order_3_G_score_rank_order_threshold | METSMITS146B_0324 | K00205 |
| PhyloNet and kwords_order_3_G_score_rank_order_threshold | METSMITS146B_0819 | K00205 |
| PhyloNet and kwords_order_3_G_score_rank_order_threshold | METSMITS146B_1139 | K00205 |
| PhyloNet and kwords_order_3_G_score_rank_order_threshold | METSMITS146C_0709 | K00205 |
| PhyloNet and kwords_order_3_G_score_rank_order_threshold | METSMITS146C_1151 | K00205 |
| PhyloNet and kwords_order_3_G_score_rank_order_threshold | METSMITS146D_1022 | K00205 |
| PhyloNet and kwords_order_3_G_score_rank_order_threshold | METSMITS146E_0322 | K00205 |
| PhyloNet and kwords_order_3_G_score_rank_order_threshold | METSMITS146E_1088 | K00205 |
| PhyloNet and kwords_order_3_G_score_rank_order_threshold | METSMITS146E_1102 | K00579 |
| PhyloNet and kwords_order_3_G_score_rank_order_threshold | METSMITS146E_1157 | K00205 |
| PhyloNet and kwords_order_3_G_score_rank_order_threshold | METSMITS146E_1543 | K00205 |
| PhyloNet and kwords_order_3_G_score_rank_order_threshold | METSMITS147A_0308 | K00205 |
| PhyloNet and kwords_order_3_G_score_rank_order_threshold | METSMITS147A_1078 | K00205 |
| PhyloNet and kwords_order_3_G_score_rank_order_threshold | METSMITS147A_1092 | K00579 |
| PhyloNet and kwords_order_3_G_score_rank_order_threshold | METSMITS147A_1146 | K00205 |
| PhyloNet and kwords_order_3_G_score_rank_order_threshold | METSMITS147A_1579 | K00205 |
| PhyloNet and kwords_order_3_G_score_rank_order_threshold | METSMITS147B_0324 | K00205 |
| PhyloNet and kwords_order_3_G_score_rank_order_threshold | METSMITS147B_1133 | K00205 |
| PhyloNet and kwords_order_3_G_score_rank_order_threshold | METSMITS147B_1147 | K00579 |
| PhyloNet and kwords_order_3_G_score_rank_order_threshold | METSMITS147B_1201 | K00205 |
| PhyloNet and kwords_order_3_G_score_rank_order_threshold | METSMITS147B_1635 | K00205 |
| PhyloNet and kwords_order_3_G_score_rank_order_threshold | METSMITS147C_0335 | K00205 |
| PhyloNet and kwords_order_3_G_score_rank_order_threshold | METSMITS147C_1016 | K00205 |
| PhyloNet and kwords_order_3_G_score_rank_order_threshold | METSMITS147C_1030 | K00579 |
| PhyloNet and kwords_order_3_G_score_rank_order_threshold | METSMITS147C_1084 | K00205 |
| PhyloNet and kwords_order_3_G_score_rank_order_threshold | METSMITS147C_1668 | K00205 |
| PhyloNet and kwords_order_3_G_score_rank_order_threshold | METSMITS94A_1012 | K00205 |
| PhyloNet and kwords_order_3_G_score_rank_order_threshold | METSMITS94A_1026 | K00579 |
| PhyloNet and kwords_order_3_G_score_rank_order_threshold | METSMITS94A_1080 | K00205 |
| PhyloNet and kwords_order_3_G_score_rank_order_threshold | METSMITS94B_1008 | K00205 |
| PhyloNet and kwords_order_3_G_score_rank_order_threshold | METSMITS94B_1023 | K00579 |
| PhyloNet and kwords_order_3_G_score_rank_order_threshold | METSMITS94B_1083 | K00205 |
| PhyloNet and kwords_order_3_G_score_rank_order_threshold | METSMITS94B_1486 | K00205 |
| PhyloNet and kwords_order_3_G_score_rank_order_threshold | METSMITS94C_0999 | K00205 |
| PhyloNet and kwords_order_3_G_score_rank_order_threshold | METSMITS94C_1013 | K00579 |
| PhyloNet and kwords_order_3_G_score_rank_order_threshold | METSMITS94C_1067 | K00205 |
| PhyloNet and kwords_order_3_G_score_rank_order_threshold | METSMITS94C_1473 | K00205 |
| PhyloNet and kwords_order_3_G_score_rank_order_threshold | METSMITS95A_1071 | K00205 |
| PhyloNet and kwords_order_3_G_score_rank_order_threshold | METSMITS95A_1087 | K00579 |
| PhyloNet and kwords_order_3_G_score_rank_order_threshold | METSMITS95A_1614 | K00205 |
| PhyloNet and kwords_order_3_G_score_rank_order_threshold | METSMITS95B_1051 | K00205 |
| PhyloNet and kwords_order_3_G_score_rank_order_threshold | METSMITS95B_1065 | K00579 |
| PhyloNet and kwords_order_3_G_score_rank_order_threshold | METSMITS95B_1120 | K00205 |
| PhyloNet and kwords_order_3_G_score_rank_order_threshold | METSMITS95C_1099 | K00205 |
| PhyloNet and kwords_order_3_G_score_rank_order_threshold | METSMITS95C_1112 | K00579 |
| PhyloNet and kwords_order_3_G_score_rank_order_threshold | METSMITS95C_1610 | K00205 |
| PhyloNet and kwords_order_3_G_score_rank_order_threshold | METSMITS95D_1007 | K00205 |
| PhyloNet and kwords_order_3_G_score_rank_order_threshold | METSMITS95D_1021 | K00579 |
| PhyloNet and kwords_order_3_G_score_rank_order_threshold | METSMITS95D_1075 | K00205 |
| PhyloNet and kwords_order_3_G_score_rank_order_threshold | METSMITS95D_1511 | K00205 |
| PhyloNet and kwords_order_3_G_score_rank_order_threshold | METSMITS96A_1037 | K00205 |
| PhyloNet and kwords_order_3_G_score_rank_order_threshold | METSMITS96A_1051 | K00579 |
| PhyloNet and kwords_order_3_G_score_rank_order_threshold | METSMITS96A_1105 | K00205 |
| PhyloNet and kwords_order_3_G_score_rank_order_threshold | METSMITS96A_1557 | K00205 |
| PhyloNet and kwords_order_3_G_score_rank_order_threshold | METSMITS96B_0967 | K00205 |
| PhyloNet and kwords_order_3_G_score_rank_order_threshold | METSMITS96B_0981 | K00579 |
| PhyloNet and kwords_order_3_G_score_rank_order_threshold | METSMITS96B_1035 | K00205 |
| PhyloNet and kwords_order_3_G_score_rank_order_threshold | METSMITS96B_1401 | K00205 |
| PhyloNet and kwords_order_3_G_score_rank_order_threshold | METSMITS96C_0321 | K00205 |
| PhyloNet and kwords_order_3_G_score_rank_order_threshold | METSMITS96C_0997 | K00205 |
| PhyloNet and kwords_order_3_G_score_rank_order_threshold | METSMITS96C_1390 | K00205 |
| PhyloNet and kwords_order_4_G_score_rank_order_threshold | METSMIALI_0037 | K00205 |
| PhyloNet and kwords_order_4_G_score_rank_order_threshold | METSMIALI_0886 | K00205 |
| PhyloNet and kwords_order_4_G_score_rank_order_threshold | METSMIALI_0955 | K00205 |
| PhyloNet and kwords_order_4_G_score_rank_order_threshold | METSMIF1_0715 | K00205 |
| PhyloNet and kwords_order_4_G_score_rank_order_threshold | METSMIF1_0784 | K00205 |
| PhyloNet and kwords_order_4_G_score_rank_order_threshold | METSMIF1_1646 | K00205 |
| PhyloNet and kwords_order_4_G_score_rank_order_threshold | METSMITS145A_0445 | K00205 |
| PhyloNet and kwords_order_4_G_score_rank_order_threshold | METSMITS145A_1086 | K00205 |
| PhyloNet and kwords_order_4_G_score_rank_order_threshold | METSMITS145A_1154 | K00205 |
| PhyloNet and kwords_order_4_G_score_rank_order_threshold | METSMITS145A_1594 | K00205 |
| PhyloNet and kwords_order_4_G_score_rank_order_threshold | METSMITS145B_0331 | K00205 |
| PhyloNet and kwords_order_4_G_score_rank_order_threshold | METSMITS145B_0824 | K00205 |
| PhyloNet and kwords_order_4_G_score_rank_order_threshold | METSMITS145B_1316 | K00205 |
| PhyloNet and kwords_order_4_G_score_rank_order_threshold | METSMITS145B_1389 | K00205 |
| PhyloNet and kwords_order_4_G_score_rank_order_threshold | METSMITS146A_0513 | K00205 |
| PhyloNet and kwords_order_4_G_score_rank_order_threshold | METSMITS146A_0828 | K00205 |
| PhyloNet and kwords_order_4_G_score_rank_order_threshold | METSMITS146A_1149 | K00205 |
| PhyloNet and kwords_order_4_G_score_rank_order_threshold | METSMITS146A_1220 | K00205 |
| PhyloNet and kwords_order_4_G_score_rank_order_threshold | METSMITS146B_0324 | K00205 |
| PhyloNet and kwords_order_4_G_score_rank_order_threshold | METSMITS146B_0819 | K00205 |
| PhyloNet and kwords_order_4_G_score_rank_order_threshold | METSMITS146B_1139 | K00205 |
| PhyloNet and kwords_order_4_G_score_rank_order_threshold | METSMITS146B_1209 | K00205 |
| PhyloNet and kwords_order_4_G_score_rank_order_threshold | METSMITS146C_0709 | K00205 |
| PhyloNet and kwords_order_4_G_score_rank_order_threshold | METSMITS146C_1151 | K00205 |
| PhyloNet and kwords_order_4_G_score_rank_order_threshold | METSMITS146C_1260 | K00205 |
| PhyloNet and kwords_order_4_G_score_rank_order_threshold | METSMITS146D_1022 | K00205 |
| PhyloNet and kwords_order_4_G_score_rank_order_threshold | METSMITS146E_0322 | K00205 |
| PhyloNet and kwords_order_4_G_score_rank_order_threshold | METSMITS146E_1088 | K00205 |
| PhyloNet and kwords_order_4_G_score_rank_order_threshold | METSMITS146E_1157 | K00205 |
| PhyloNet and kwords_order_4_G_score_rank_order_threshold | METSMITS146E_1543 | K00205 |
| PhyloNet and kwords_order_4_G_score_rank_order_threshold | METSMITS147A_0308 | K00205 |
| PhyloNet and kwords_order_4_G_score_rank_order_threshold | METSMITS147A_1078 | K00205 |
| PhyloNet and kwords_order_4_G_score_rank_order_threshold | METSMITS147A_1146 | K00205 |
| PhyloNet and kwords_order_4_G_score_rank_order_threshold | METSMITS147A_1579 | K00205 |
| PhyloNet and kwords_order_4_G_score_rank_order_threshold | METSMITS147B_0324 | K00205 |
| PhyloNet and kwords_order_4_G_score_rank_order_threshold | METSMITS147B_1133 | K00205 |
| PhyloNet and kwords_order_4_G_score_rank_order_threshold | METSMITS147B_1201 | K00205 |
| PhyloNet and kwords_order_4_G_score_rank_order_threshold | METSMITS147B_1570 | K00205 |
| PhyloNet and kwords_order_4_G_score_rank_order_threshold | METSMITS147B_1635 | K00205 |
| PhyloNet and kwords_order_4_G_score_rank_order_threshold | METSMITS147C_0335 | K00205 |
| PhyloNet and kwords_order_4_G_score_rank_order_threshold | METSMITS147C_0845 | K00205 |
| PhyloNet and kwords_order_4_G_score_rank_order_threshold | METSMITS147C_1016 | K00205 |
| PhyloNet and kwords_order_4_G_score_rank_order_threshold | METSMITS147C_1084 | K00205 |
| PhyloNet and kwords_order_4_G_score_rank_order_threshold | METSMITS147C_1668 | K00205 |
| PhyloNet and kwords_order_4_G_score_rank_order_threshold | METSMITS94A_1012 | K00205 |
| PhyloNet and kwords_order_4_G_score_rank_order_threshold | METSMITS94A_1080 | K00205 |
| PhyloNet and kwords_order_4_G_score_rank_order_threshold | METSMITS94B_1008 | K00205 |
| PhyloNet and kwords_order_4_G_score_rank_order_threshold | METSMITS94B_1083 | K00205 |
| PhyloNet and kwords_order_4_G_score_rank_order_threshold | METSMITS94B_1486 | K00205 |
| PhyloNet and kwords_order_4_G_score_rank_order_threshold | METSMITS94C_0999 | K00205 |
| PhyloNet and kwords_order_4_G_score_rank_order_threshold | METSMITS94C_1067 | K00205 |
| PhyloNet and kwords_order_4_G_score_rank_order_threshold | METSMITS94C_1473 | K00205 |
| PhyloNet and kwords_order_4_G_score_rank_order_threshold | METSMITS95A_1071 | K00205 |
| PhyloNet and kwords_order_4_G_score_rank_order_threshold | METSMITS95A_1143 | K00205 |
| PhyloNet and kwords_order_4_G_score_rank_order_threshold | METSMITS95A_1614 | K00205 |
| PhyloNet and kwords_order_4_G_score_rank_order_threshold | METSMITS95B_1051 | K00205 |
| PhyloNet and kwords_order_4_G_score_rank_order_threshold | METSMITS95B_1120 | K00205 |
| PhyloNet and kwords_order_4_G_score_rank_order_threshold | METSMITS95C_1099 | K00205 |
| PhyloNet and kwords_order_4_G_score_rank_order_threshold | METSMITS95C_1166 | K00205 |
| PhyloNet and kwords_order_4_G_score_rank_order_threshold | METSMITS95C_1610 | K00205 |
| PhyloNet and kwords_order_4_G_score_rank_order_threshold | METSMITS95D_1007 | K00205 |
| PhyloNet and kwords_order_4_G_score_rank_order_threshold | METSMITS95D_1075 | K00205 |
| PhyloNet and kwords_order_4_G_score_rank_order_threshold | METSMITS95D_1511 | K00205 |
| PhyloNet and kwords_order_4_G_score_rank_order_threshold | METSMITS96A_1037 | K00205 |
| PhyloNet and kwords_order_4_G_score_rank_order_threshold | METSMITS96A_1105 | K00205 |
| PhyloNet and kwords_order_4_G_score_rank_order_threshold | METSMITS96A_1557 | K00205 |
| PhyloNet and kwords_order_4_G_score_rank_order_threshold | METSMITS96B_0967 | K00205 |
| PhyloNet and kwords_order_4_G_score_rank_order_threshold | METSMITS96B_1035 | K00205 |
| PhyloNet and kwords_order_4_G_score_rank_order_threshold | METSMITS96B_1401 | K00205 |
| PhyloNet and kwords_order_4_G_score_rank_order_threshold | METSMITS96C_0997 | K00205 |
| PhyloNet and kwords_order_4_G_score_rank_order_threshold | METSMITS96C_1390 | K00205 |
| PhyloNet and kwords_order_5_G_score_rank_order_threshold | METSMITS146E_1543 | K00205 |
| PhyloNet and kwords_order_5_G_score_rank_order_threshold | METSMITS147B_1635 | K00205 |
| PhyloNet and kwords_order_5_G_score_rank_order_threshold | METSMITS147C_1668 | K00205 |

### Example 10

### Expression Profiling of M. smithii Strains by RNA-Seq

RNA-Seq was used to profile the transcriptomes of five of the M. smithii isolates: One from each member of the MZ family, one from each of the DZ co-twins, plus the PS type strain. The five strains from the two families were chosen because SNP, OGU, and EC analyses indicated that these isolates were representative of the strains from their human hosts, and because they exhibited consistent patterns of growth on MBC medium containing 2.8 or 44.1 mM formate, a substrate for the first enzyme involved in the methanogenesis pathway, formate dehydrogenase (EC 1.2.1.2 in FIG. 24B). Triplicate cultures were grown to midlog phase in medium with either low or high formate concentrations under an atmosphere that contained 80% hydrogen. Total RNA was extracted, structural RNAs were depleted, and double-stranded cDNA was synthesized and sequenced with an Illumina GA-IIx instrument (36-nt reads; 3-4 million reads per sample, with each biological triplicate sequenced twice as technical replicates). Reads were normalized to reads per kilobase per million (RPKM) and mapped back to each strain's own reference genome. At midlog phase, the number of protein-coding genes with ≧10 mapped mRNA-derived reads varied from 1,594 to 1,782 (89-97% of all CDS) among the 5 strains (Table 29). When the 987 OGUs that comprise the conserved core of the M. smithii pan-genome were compared to 31 sequenced methanogens associated with the human gut (M. stadmanae), cow rumen (M. ruminantium) or various environmental habitats, 55 OGUs were identified as unique to M. smithii (Blastp threshold E<10⁻¹⁰), of which 42 encoded predicted conserved hypothetical or hypothetical proteins (Table 30). At the depth of sequencing achieved, RNA-Seq indicated that 34 of these 42 hypothetical genes were expressed in midlog phase in the PS type strain (Table 30).

Next the phenotypes of strains based on normalized expression of each gene encoding each EC were compared. Examining the gene expression data across functional groups allowed the strains to be compared: The results revealed that no gene family was consistently regulated by formate across all strains. To identify genes significantly regulated by formate in each strain, normalized reads with CyberT were first analyzed. Two criteria were used for determining significance in regulation: a posterior probability of differential expression (PPDE) threshold ≧0.97, and a ≧2-fold difference in expression (either direction) when a given strain was incubated in low versus high levels of formate (Table 31).

All of the genes in the methanogenesis pathway illustrated in FIG. 24C were expressed in all six strains. Nonetheless, several of the genes in this pathway exhibited strain-specific differences in their levels of expression including EC 1.5.99.9 (F420-dependent methylene tetrahydromethanopterin dehydrogenase) and EC 1.5.99.11 (5,10-methylenetetrahydromethanopterin reductase). Cobalt, an important cofactor for some of the enzymes in the methanogenesis pathway, is translocated by an ABC transporter: Components of the transporter exhibited formate-responsive behavior in the PS type strain and in the strain from one of the DZ co-twins (TS145) but not in the strains from her sister or mother (Table 31).

Looking beyond the methanogenesis pathway, none of the genes encoding ECs in the M. smithii pan-genome satisfied our criteria for being responsive to differences in formate levels in the medium at midlog phase in all strains. However, as with components of the methanogenesis pathway, some exhibited strain-specific differences in formate sensitivity e.g., in strain METSMITS145B (from DZ co-twin 1) genes encoding the subunits of MtrH (EC 2.1.1.86; tetrahydromethanopterin S-methyltransferase) were up-regulated in high formate, whereas in strain METSMITS146E (from the sister of DZ co-twin 1) they were down-regulated (see Table 31 for additional examples).

M. smithii uses ammonia as a nitrogen source via an energy-dependent glutamine synthetase-glutamate synthase pathway, which has high affinity for ammonia, and a ATP-independent pathway with lower affinity (FIG. 17A). Both pathways are expressed in all strains, with 0.4-1.21% of reads mapping to enzymes involved in assimilation of ammonia. The energy-dependent GlnA pathway is generally expressed at a much higher level than the low affinity pathway, although strain-specific differences in levels expression were noted. With few exceptions, such as the genes encoding EC 1.4.1.4 and EC 1.4.1.13 in strains METSMITS145B and METSMITS96A, components of both pathways failed to exhibit a significant difference in their levels of expression in any of the strains as a function of formate concentration. Another exception was the ammonium transporter (AmtB) (FIGS. 17 B and C and Table 31).

Using the threshold criteria for formate-responsive expression, four of the six strains were defined as having genes that were sensitive to levels of this compound. Table 31 lists the 9 genes present in type strain PS, the 340 genes in the strain recovered from the mother of the DZ co-twins (TS145), the 23 genes in the strain isolated from one of her daughters (TS146), and the 81 genes in the strain from the mother of the MZ twins (TS96). Intriguingly, no genes were identified in strains from MZ twins of this mother (TS94, TS95) that exhibited significant formate responsiveness. The core component of M. smithii's pan-genome contained no genes that met our criteria for formate-responsive behavior in every isolate.

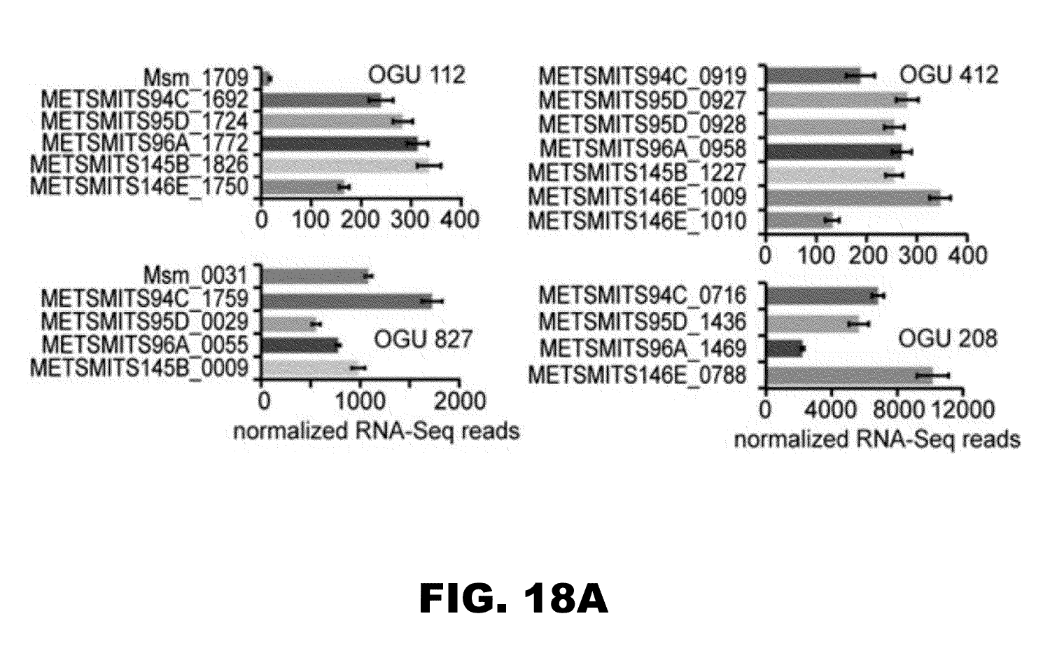

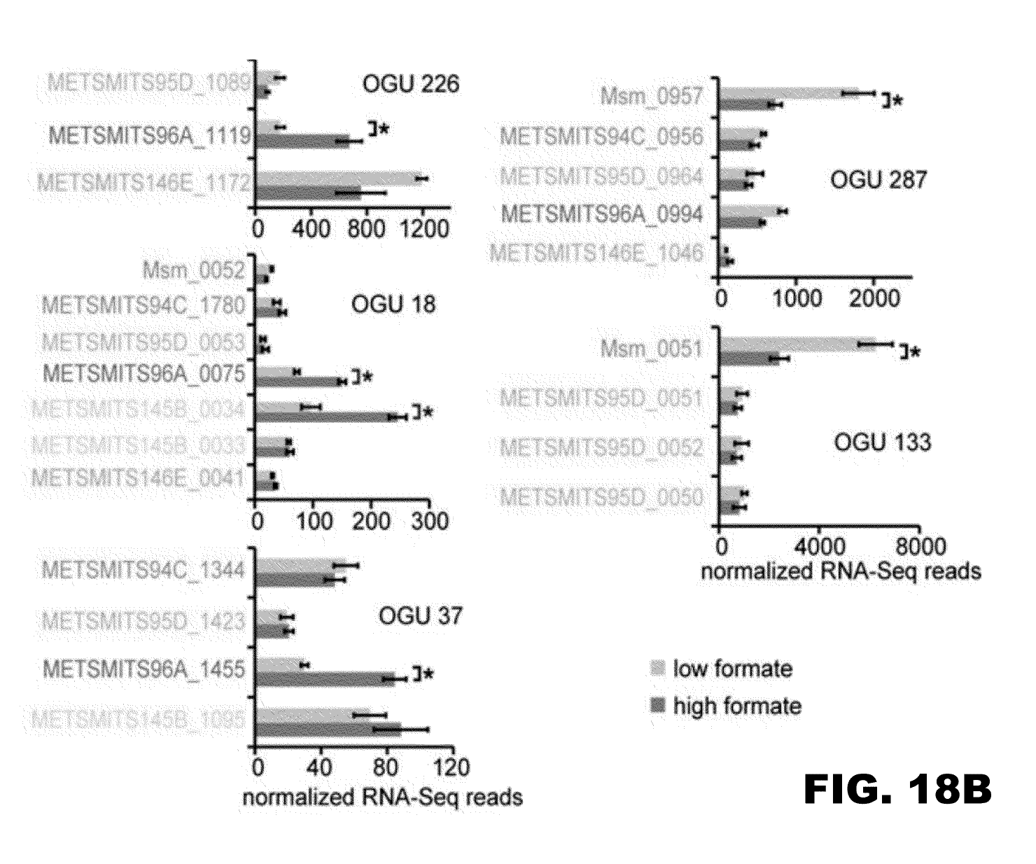

The utility of using formate to identify strain-specific phenotypes is best illustrated by ALPs. As noted above, each sequenced strain contained a distinctive repertoire of genes encoding ALPs, with only 6 ALP OGUs shared by all isolates. ALP OGUs 112, 208, 412, and 827 are encoded by genes present in 4-6 of the strains: None of the genes are formate-responsive but members of each OGU exhibit strain-specific differences in their levels of expression (levels of expression are also notably different between ALP OGUs). OGUs 18, 37, 133, and 226 show strain-specific differences in their representation, strain-specific differences in their levels of expression, plus within-OGU differences in their formate sensitivity (FIG. 18).

TABLE 29
Overview of RNA-Seq dataset

| strain | fraction_CDS | number_CDS | total_mapped | total_reads |
|---|---|---|---|---|
| METSMITS94C | 0.0294 | 93481 | 3302490 | 3429629 |
| METSMITS95D | 0.04526 | 138514 | 3170100 | 3278630 |
| METSMITS96A | 0.06311 | 234981 | 3994000 | 4095260 |
| METSMITS145B | 0.05809 | 153439 | 2873157 | 3116025 |
| METSMITS146E | 0.08068 | 190337 | 2756408 | 2895607 |
| MsmPS | 0.1027 | 219511 | 2621639 | 2713609 |
| overall | 0.06321 | 171710 | 3119632 | 3254793 |

Average number of reads assigned to protein coding regions (CDS), the total number of mapped reads, and the total number of reads for each strain, averaged across all samples for that strain.

TABLE 30
OGUs present in the M. smithii core genome but not in other sequence methanogens*

| | | mRNA |
|---|---|---|
| | | detected |
| Cluster | Annotation (M. smithii type strain) | in vitro? |
| Cluster 1042 | hypothetical protein Msm_0799 [Methanobrevibacter smithii ATCC 35061] | yes |
| Cluster 1066 | hypothetical protein Msm_0212 [Methanobrevibacter smithii ATCC 35061] | yes |
| Cluster 1086 | hypothetical protein Msm_0258 [Methanobrevibacter smithii ATCC 35061] | yes |
| Cluster 1102 | O-linked GlcNAc transferase [Methanobrevibacter smithii ATCC 35061] | |
| Cluster 1114 | hypothetical protein Msm_0067 [Methanobrevibacter smithii ATCC 35061] | yes |
| Cluster 1145 | hypothetical protein Msm_1152 [Methanobrevibacter smithii ATCC 35061] | yes |
| Cluster 1260 | acetylesterase [Methanobrevibacter smithii ATCC 35061] | |
| Cluster 1348 | hypothetical protein Msm_1729 [Methanobrevibacter smithii ATCC 35061] | yes |
| Cluster 1388 | putative SAM-dependent methyltransferase [Methanobrevibacter smithii | |
| | ATCC 35061] | |
| Cluster 1414 | cobalt ABC transporter, permease component, CbiQ [Methanobrevibacter smithii | |
| | ATCC 35061] | |
| Cluster 1463 | hypothetical protein Msm_0499 [Methanobrevibacter smithii ATCC 35061] | yes |
| Cluster 1483 | hypothetical protein Msm_0529 [Methanobrevibacter smithii ATCC 35061] | yes |
| Cluster 1503 | hypothetical protein Msm_1205 [Methanobrevibacter smithii ATCC 35061] | yes |
| Cluster 1510 | putative calcium-binding protein [Methanobrevibacter smithii ATCC 35061] | |
| Cluster 1641 | hypothetical protein Msm_1696 [Methanobrevibacter smithii ATCC 35061] | yes |
| Cluster 1665 | hypothetical protein Msm_1458 [Methanobrevibacter smithii ATCC 35061] | yes |
| Cluster 1672 | major facilitator superfamily permease [Methanobrevibacter smithii ATCC 35061] | |
| Cluster 1826 | hypothetical protein Msm_0259 [Methanobrevibacter smithii ATCC 35061] | yes |
| Cluster 1876 | hypothetical protein Msm_1490 [Methanobrevibacter smithii ATCC 35061] | yes |
| Cluster 1883 | hypothetical protein Msm_1571 [Methanobrevibacter smithii ATCC 35061] | yes |
| Cluster 1888 | hypothetical protein Msm_0546 [Methanobrevibacter smithii ATCC 35061] | yes |
| Cluster 1933 | hypothetical protein Msm_1199 [Methanobrevibacter smithii ATCC 35061] | yes |
| Cluster 1943 | hypothetical protein Msm_1470 [Methanobrevibacter smithii ATCC 35061] | Marginal/no |
| | | expression |
| Cluster 2011 | hypothetical protein Msm_0003 [Methanobrevibacter smithii ATCC 35061] | yes |
| Cluster 2016 | hypothetical protein Msm_0698 [Methanobrevibacter smithii ATCC 35061] | yes |
| Cluster 2030 | hypothetical protein Msm_0180 [Methanobrevibacter smithii ATCC 35061] | yes |
| Cluster 2035 | hypothetical protein Msm_1255 [Methanobrevibacter smithii ATCC 35061] | yes |
| Cluster 2052 | hypothetical protein Msm_0712 [Methanobrevibacter smithii ATCC 35061] | yes |
| Cluster 2069 | hypothetical protein Msm_1509 [Methanobrevibacter smithii ATCC 35061] | yes |
| Cluster 2089 | hypothetical protein Msm_0454 [Methanobrevibacter smithii ATCC 35061] | yes |
| Cluster 2134 | hypothetical protein Msm_0139 [Methanobrevibacter smithii ATCC 35061] | yes |
| Cluster 2169 | hypothetical protein Msm_0098 [Methanobrevibacter smithii ATCC 35061] | yes |
| Cluster 2174 | hypothetical protein Msm_0005 [Methanobrevibacter smithii ATCC 35061] | yes |
| Cluster 2181 | hypothetical protein Msm_0442 [Methanobrevibacter smithii ATCC 35061] | yes |
| Cluster 2206 | hypothetical protein Msm_0211 [Methanobrevibacter smithii ATCC 35061] | yes |
| Cluster 2269 | hypothetical protein Msm_0667 [Methanobrevibacter smithii ATCC 35061] | yes |
| Cluster 2299 | hypothetical protein Msm_1697 [Methanobrevibacter smithii ATCC 35061] | yes |
| Cluster 2338 | hypothetical protein Msm_0685 [Methanobrevibacter smithii ATCC 35061] | Marginal |
| Cluster 2390 | hypothetical protein Msm_1563 [Methanobrevibacter smithii ATCC 35061] | yes |
| Cluster 2402 | putative monovalent cation/H+ antiporter subunit F [Methanobrevibacter smithii ATCC | |
| | 35061] | |
| Cluster 2427 | hypothetical protein Msm_0366 [Methanobrevibacter smithii ATCC 35061] | yes |
| Cluster 2491 | hypothetical protein Msm_0478 [Methanobrevibacter smithii ATCC 35061] | Marginal |
| Cluster 2521 | hypothetical protein Msm_0587 [Methanobrevibacter smithii ATCC 35061] | yes |
| Cluster 2545 | hypothetical protein Msm_1605 [Methanobrevibacter smithii ATCC 35061] | Marginal |
| Cluster 2579 | hypothetical protein Msm_0658 [Methanobrevibacter smithii ATCC 35061] | Marginal |
| Cluster 2590 | hypothetical protein Msm_0278 [Methanobrevibacter smithii ATCC 35061] | Marginal |
| Cluster 2595 | ferredoxin [Methanobrevibacter smithii ATCC 35061] | |
| Cluster 2597 | preprotein translocase subunit SecE [Methanobrevibacter smithii ATCC 35061] | |
| Cluster 2606 | hypothetical protein Msm_1163 [Methanobrevibacter smithii ATCC 35061] | Marginal |
| Cluster 2617 | hypothetical protein Msm_0782 [Methanobrevibacter smithii ATCC 35061] | yes |
| Cluster 2807 | rubredoxin [Methanobrevibacter smithii ATCC 35061] | |
| Cluster 551 | glycerol-3-phosphate cytidyltransferase, TagD [Methanobrevibacter smithii ATCC | |
| | 35061] | |
| Cluster 573 | hypothetical protein Msm_1543 [Methanobrevibacter smithii ATCC 35061] | yes |
| Cluster 591 | ATPase [Methanobrevibacter smithii ATCC 35061] | |
| Cluster 810 | integrase-recombinase protein [Methanobrevibacter smithii ATCC 35061] | |

*Blastp threshold E < 10-10; methanogenic species used for the analysis: Methanobrevibacter_ruminantium_M1, Methanocaldococcus_FS406_22, Methanocaldococcus_fervens_AG86, Methanocaldococcus_infernus_ME, Methanocaldococcus_jannaschii_DSM_2661, Methanocaldococcus_vulcanius_M7, Methanocella_paludicola_SANAE, Methanococcoides_burtonii_DSM_6242, Methanococcus_aeolicus_Nankai_3, Methanococcus_maripaludis_C5, Methanococcus_maripaludis_C6, Methanococcus_maripaludis_C7, Methanococcus_maripaludis_S2, Methanococcus_vannielii_SB, Methanococcus_voltae_A3, Methanocorpusculum_labreanum_Z, Methanoculleus_marisnigri_JR1, Methanohalobium_evestigatum_Z_7303, Methanohalophilus_mahii_DSM_5219, Methanoplanus_petrolearius_DSM_11571, Methanopyrus_kandleri_AV19, Methanosaeta_thermophila_PT, Methanosarcina_acetivorans_C2A, Methanosarcina_barkeri_Fusaro, Methanosarcina_mazei_Go1, Methanosphaera_stadtmanae_DSM_3091, Methanosphaerula_palustris_E1_9c, Methanospirillum_hungatei_JF_1, Methanothermobacter_marburgensis_Marburg, Methanothermobacter_thermautotrophicus_Delta_H, Methanothermus_fervidus_DSM_2088. Genome sequences were downloaded from NCBI website

TABLE 31
Genes regulated by formate concentration by strain

| | | Normalized RNA-Seq counts | | | |
|---|---|---|---|---|---|
| | | High | Low | fold | |
| Gene | Annotation | formate | formate | change | PPDE(p) |
| Msm1453 | hypothetical protein | 90.1 | 253.2 | 2.81 | 0.9929 |
| Msm1119 | hypothetical protein | 1965.4 | 3984.0 | 2.03 | 0.9918 |
| Msm1488 | cobalt ABC transporter, permease | 869.1 | 1763.4 | 2.03 | 0.9902 |
| | component, CbiM | | | | |
| Msm1649 | hypothetical protein | 126.8 | 39.6 | −3.20 | 0.9858 |
| Msm0585 | cobalt ABC transporter, permease | 131.5 | 320.6 | 2.44 | 0.9853 |
| | component, CbiQ | | | | |
| Msm1306 | adhesin-like protein (Cluster 86) | 93.3 | 208.0 | 2.23 | 0.9841 |
| Msm0957 | adhesin-like protein (Cluster 287) | 731.2 | 1805.1 | 2.47 | 0.9759 |
| Msm0051 | adhesin-like protein (Cluster 133) | 2412.7 | 6249.6 | 2.59 | 0.9755 |
| Msm1747 | type II restriction enzyme, methylase | 52.6 | 110.5 | 2.10 | 0.9722 |
| | subunit | | | | |
| METSMITS146E_0738 | hypothetical protein | 3740.8 | 1409.7 | −2.65 | 0.9999 |
| METSMITS146E_0960 | GTPase of unknown function | 430.6 | 185.3 | −2.32 | 0.9994 |
| METSMITS146E_1448 | 4Fe—4S binding domain | 6952.5 | 13982.6 | 2.01 | 0.9973 |
| METSMITS146E_0599 | ABC transporter | 88.7 | 30.9 | −2.87 | 0.9967 |
| METSMITS146E_0243 | hypothetical protein | 59.3 | 233.1 | 3.93 | 0.9953 |
| METSMITS146E_0461 | hypothetical protein | 267.2 | 73.7 | −3.63 | 0.9948 |
| METSMITS146E_1097 | Tetrahydromethanopterin S- | 3051.2 | 7097.8 | 2.33 | 0.9942 |
| | methyltransferase | | | | |
| METSMITS146E_0307 | Carboxymuconolactone decarboxylase | 3318.9 | 1513.6 | −2.19 | 0.9934 |
| | family | | | | |
| METSMITS146E_1103 | Tetrahydromethanopterin S- | 2099.9 | 5557.2 | 2.65 | 0.9883 |
| | methyltransferase, | | | | |
| METSMITS146E_0686 | eRF1 domain 1 | 335.3 | 143.8 | −2.33 | 0.9875 |
| METSMITS146E_0385 | Alcohol dehydrogenase GroES-like domain | 357.5 | 172.6 | −2.07 | 0.9872 |
| METSMITS146E_0783 | Formate/nitrite transporter | 4542.5 | 11725.6 | 2.58 | 0.9862 |
| METSMITS146E_1104 | Tetrahydromethanopterin S- | 2314.3 | 5551.7 | 2.40 | 0.9861 |
| | methyltransferase, | | | | |
| METSMITS146E_1121 | N2,N2-dimethylguanosine tRNA | 290.6 | 137.6 | −2.11 | 0.9840 |
| | methyltransfera | | | | |
| METSMITS146E_0493 | hypothetical protein | 65.0 | 12.0 | −5.43 | 0.9817 |
| METSMITS146E_1244 | hypothetical protein | 208.9 | 90.1 | −2.32 | 0.9778 |
| METSMITS146E_1854 | YLP motif | 34.0 | 9.7 | −3.49 | 0.9753 |
| METSMITS146E_1202 | Bacterial regulatory protein, arsR family | 1344.2 | 326.5 | −4.12 | 0.9744 |
| METSMITS146E_1583 | MarR family | 2807.7 | 1332.7 | −2.11 | 0.9733 |
| METSMITS146E_0848 | Fibronectin-binding protein A N-terminus | 142.8 | 69.0 | −2.07 | 0.9732 |
| | (Fb | | | | |
| METSMITS146E_0278 | Pyridoxal-phosphate dependent enzyme | 80.5 | 33.0 | −2.44 | 0.9726 |
| METSMITS146E_1163 | NADH-ubiquinone/plastoquinone | 527.8 | 1067.8 | 2.02 | 0.9714 |
| | oxidoreduct | | | | |
| METSMITS146E_1164 | hypothetical protein | 262.4 | 529.8 | 2.02 | 0.9707 |
| METSMITS145B_0176 | Lyase | 352.2 | 113.7 | −3.10 | 0.9999999 |
| METSMITS145B_1436 | Chlamydia polymorphic membrane protein | 298.6 | 30.9 | −9.68 | 0.9999996 |
| | (Chl | | | | |
| METSMITS145B_0056 | tRNA synthetases class I (M) | 1177.8 | 308.9 | −3.81 | 0.9999988 |
| METSMITS145B_1144 | Peptidase family M50 | 289.6 | 95.7 | −3.03 | 0.9999983 |
| METSMITS145B_0784 | hypothetical protein | 130.9 | 454.8 | 3.47 | 0.9999942 |
| METSMITS145B_1676 | Thiolase, C-terminal domain | 2052.7 | 1019.5 | −2.01 | 0.9999928 |
| METSMITS145B_1188 | hypothetical protein | 4813.1 | 911.2 | −5.28 | 0.9999909 |
| METSMITS145B_0880 | Ribosomal protein S5, N-terminal domai | 563.5 | 145.1 | −3.88 | 0.9999902 |
| METSMITS145B_1454 | Protein of unknown function DUF75 | 362.4 | 112.3 | −3.23 | 0.9999869 |
| METSMITS145B_1212 | KH domain | 2266.0 | 536.7 | −4.22 | 0.9999837 |
| METSMITS145B_0374 | hypothetical protein | 817.0 | 295.3 | −2.77 | 0.9999826 |
| METSMITS145B_1216 | RNA polymerase Rpb2, domain 6 | 457.5 | 92.3 | −4.96 | 0.9999824 |
| METSMITS145B_0870 | TruB family pseudouridylate synthase (N | 261.4 | 45.0 | −5.81 | 0.9999807 |
| | term | | | | |
| METSMITS145B_0187 | Nucleoside diphosphate kinase | 1403.1 | 224.9 | −6.24 | 0.9999804 |
| METSMITS145B_0847 | GHMP kinases N terminal domain | 279.7 | 83.4 | −3.35 | 0.9999792 |
| METSMITS145B_0067 | Thiamine pyrophosphate enzyme, C- | 1382.7 | 387.2 | −3.57 | 0.9999792 |
| | termina | | | | |
| METSMITS145B_0414 | Permease family | 515.1 | 186.2 | −2.77 | 0.9999771 |
| METSMITS145B_0185 | Ribosomal protein S6e | 677.9 | 155.1 | −4.37 | 0.9999756 |
| METSMITS145B_1306 | Ribosomal protein L16p/L10e | 1838.1 | 517.9 | −3.55 | 0.9999755 |
| METSMITS145B_0901 | Ribosomal protein L3 | 894.0 | 183.9 | −4.86 | 0.9999753 |
| METSMITS145B_0387 | Glutamine amidotransferases class-II | 726.5 | 116.9 | −6.21 | 0.9999752 |
| METSMITS145B_0644 | Ribosomal protein L10 | 5205.4 | 905.5 | −5.75 | 0.9999750 |
| METSMITS145B_0184 | Elongation factor Tu GTP binding domain | 851.1 | 202.3 | −4.21 | 0.9999750 |
| METSMITS145B_1737 | Cobalt transport protein component CbiN | 6656.3 | 2065.1 | −3.22 | 0.9999708 |
| METSMITS145B_0799 | Ribosomal protein S8e | 4787.9 | 895.1 | −5.35 | 0.9999677 |
| METSMITS145B_1215 | RNA polymerase Rpb1, domain 2 | 508.8 | 133.9 | −3.80 | 0.9999634 |
| METSMITS145B_1438 | CobN/Magnesium Chelatase | 489.4 | 194.0 | −2.52 | 0.9999603 |
| METSMITS145B_1077 | Hsp20/alpha crystallin family | 2923.3 | 5909.2 | 2.02 | 0.9999597 |
| METSMITS145B_0242 | MarR family | 2774.0 | 7441.9 | 2.68 | 0.9999571 |
| METSMITS145B_1847 | hypothetical protein | 164.0 | 17.6 | −9.29 | 0.9999571 |
| METSMITS145B_0895 | KH domain | 543.1 | 145.8 | −3.72 | 0.9999558 |
| METSMITS145B_0385 | Conserved region in glutamate synthase | 1020.6 | 257.5 | −3.96 | 0.9999548 |
| METSMITS145B_0920 | Fibronectin-binding protein A N-terminus | 143.5 | 59.5 | −2.41 | 0.9999506 |
| | (Fb | | | | |
| METSMITS145B_1828 | Ferritin-like domain | 4084.7 | 13266.8 | 3.25 | 0.9999480 |
| METSMITS145B_0055 | Protein of unknown function (DUF530) | 590.1 | 158.8 | −3.72 | 0.9999469 |
| METSMITS145B_1456 | Eukaryotic translation initiation factor | 652.6 | 214.8 | −3.04 | 0.9999450 |
| METSMITS145B_1202 | Elongation factor Tu GTP binding domain | 1306.1 | 319.2 | −4.09 | 0.9999449 |
| METSMITS145B_0645 | Ribosomal protein L1p/L10e family | 6968.3 | 1572.0 | −4.43 | 0.9999448 |
| METSMITS145B_1217 | RNA polymerase beta subunit | 553.0 | 113.1 | −4.89 | 0.9999402 |
| METSMITS145B_0860 | Ribosomal protein S13/S18 | 1428.8 | 335.2 | −4.26 | 0.9999378 |
| METSMITS145B_0585 | Binding-protein-dependent transport syst | 1242.9 | 168.7 | −7.37 | 0.9999323 |
| METSMITS145B_0125 | M42 glutamyl aminopeptidase | 1233.1 | 419.3 | −2.94 | 0.9999312 |
| METSMITS145B_1525 | Protein of unknown function (DUF521) | 785.5 | 268.9 | −2.92 | 0.9999263 |
| METSMITS145B_1214 | RNA polymerase Rpb1, domain 5 | 2850.7 | 471.7 | −6.04 | 0.9999254 |
| METSMITS145B_0060 | Eukaryotic and archaeal DNA primase sma | 589.9 | 282.4 | −2.09 | 0.9999223 |
| METSMITS145B_0655 | Tetrahydromethanopterin S- | 3532.9 | 573.1 | −6.16 | 0.9999221 |
| | methyltransferase | | | | |
| METSMITS145B_1569 | Carbonic anhydrase | 5284.9 | 11711.4 | 2.22 | 0.9999208 |
| METSMITS145B_1433 | DnaJ domain | 351.4 | 114.3 | −3.07 | 0.9999192 |
| METSMITS145B_0851 | Enolase, C-terminal TIM barrel domain | 243.2 | 50.1 | −4.86 | 0.9999189 |
| METSMITS145B_1203 | Ribosomal protein S7p/S5e | 863.9 | 222.6 | −3.88 | 0.9999147 |
| METSMITS145B_0646 | Ribosomal protein L11, N-terminal dom | 2943.1 | 909.8 | −3.24 | 0.9999070 |
| METSMITS145B_0065 | Radical SAM superfamily | 1138.1 | 394.6 | −2.88 | 0.9999007 |
| METSMITS145B_1200 | Ribosomal protein S10p/S20e | 1683.7 | 511.8 | −3.29 | 0.9998999 |
| METSMITS145B_0845 | FMN-dependent dehydrogenase | 184.0 | 47.6 | −3.87 | 0.9998983 |
| METSMITS145B_0317 | Ribosomal L15 | 2318.5 | 804.7 | −2.88 | 0.9998920 |
| METSMITS145B_0584 | hypothetical protein | 603.8 | 65.7 | −9.19 | 0.9998727 |
| METSMITS145B_0780 | MotA/TolQ/ExbB proton channel family | 203.1 | 547.6 | 2.70 | 0.9998725 |
| METSMITS145B_0053 | hypothetical protein | 415.8 | 1428.8 | 3.44 | 0.9998667 |
| METSMITS145B_0126 | Coenzyme F420 | 1760.1 | 505.3 | −3.48 | 0.9998661 |
| | hydrogenase/dehydrogenase, | | | | |
| METSMITS145B_0582 | ABC transporter | 981.0 | 134.7 | −7.28 | 0.9998638 |
| METSMITS145B_1526 | DHH family | 434.8 | 102.7 | −4.23 | 0.9998629 |
| METSMITS145B_0973 | TCP-1/cpn60 chaperonin family | 2008.9 | 543.5 | −3.70 | 0.9998577 |
| METSMITS145B_1168 | hypothetical protein | 152.5 | 43.1 | −3.54 | 0.9998449 |
| METSMITS145B_0857 | RNA polymerase Rpb3/RpoA insert | 917.6 | 186.2 | −4.93 | 0.9998397 |
| | domain | | | | |
| METSMITS145B_0249 | DHH family | 387.9 | 186.1 | −2.08 | 0.9998334 |
| METSMITS145B_0763 | Glutamine synthetase, catalytic domain | 3520.8 | 1064.5 | −3.31 | 0.9998141 |
| METSMITS145B_1495 | hypothetical protein | 22.7 | 55.2 | 2.44 | 0.9998125 |
| METSMITS145B_1572 | Aspartate/ornithine carbamoyltransferase, | 475.6 | 149.2 | −3.19 | 0.9998077 |
| | As | | | | |
| METSMITS145B_0709 | Aminotransferase class-V | 3124.1 | 1317.0 | −2.37 | 0.9998010 |
| METSMITS145B_0504 | CBS domain pair | 844.3 | 420.0 | −2.01 | 0.9997991 |
| METSMITS145B_0186 | Elongation factor Tu GTP binding domain | 1355.8 | 217.8 | −6.22 | 0.9997922 |
| METSMITS145B_1383 | hypothetical protein | 257.4 | 72.7 | −3.54 | 0.9997910 |
| METSMITS145B_0739 | Ribosomal protein S19e | 1414.1 | 295.0 | −4.79 | 0.9997828 |
| METSMITS145B_0876 | hypothetical protein | 564.2 | 184.2 | −3.06 | 0.9997722 |
| METSMITS145B_1314 | adhesin-like protein (Cluster 199) | 181.4 | 41.6 | −4.36 | 0.9997633 |
| METSMITS145B_1189 | Glutamate/Leucine/Phenylalanine/Valin | 3763.6 | 842.9 | −4.46 | 0.9997607 |
| METSMITS145B_0737 | hypothetical protein | 1476.0 | 249.9 | −5.91 | 0.9997553 |
| METSMITS145B_0783 | Cna protein B-type domain | 406.0 | 2803.3 | 6.90 | 0.9997532 |
| METSMITS145B_0855 | Ribosomal protein L13 | 817.1 | 154.2 | −5.30 | 0.9997492 |
| METSMITS145B_1213 | Ribosomal protein L7Ae/L30e/S12e/Gadd4 | 2448.7 | 494.7 | −4.95 | 0.9997411 |
| METSMITS145B_0750 | haloacid dehalogenase-like hydrolase | 1233.6 | 352.2 | −3.50 | 0.9997362 |
| METSMITS145B_0388 | SNO glutamine amidotransferase family | 987.2 | 172.5 | −5.72 | 0.9997182 |
| METSMITS145B_0486 | Mov34/MPN/PAD-1 family | 164.5 | 61.4 | −2.68 | 0.9997154 |
| METSMITS145B_0586 | hypothetical protein | 5082.5 | 618.0 | −8.22 | 0.9997120 |
| METSMITS145B_0814 | CoA binding domain | 1264.6 | 377.6 | −3.35 | 0.9997018 |
| METSMITS145B_0188 | Ribosomal protein L24e | 1296.2 | 242.6 | −5.34 | 0.9996973 |
| METSMITS145B_1747 | IMP dehydrogenase/GMP reductase | 484.8 | 161.0 | −3.01 | 0.9996970 |
| | domain | | | | |
| METSMITS145B_0066 | hypothetical protein | 2898.0 | 780.7 | −3.71 | 0.9996914 |
| METSMITS145B_1665 | hypothetical protein | 154.4 | 380.2 | 2.46 | 0.9996806 |
| METSMITS145B_0858 | Ribosomal protein S11 | 909.4 | 201.7 | −4.51 | 0.9996784 |
| METSMITS145B_0902 | Uncharacterized ACR, COG2106 | 1763.8 | 633.2 | −2.79 | 0.9996432 |
| METSMITS145B_0449 | BioY family | 1347.2 | 570.2 | −2.36 | 0.9996428 |
| METSMITS145B_1776 | hypothetical protein | 270.2 | 775.6 | 2.87 | 0.9996396 |
| METSMITS145B_0995 | Aconitase C-terminal domain | 435.5 | 153.2 | −2.84 | 0.9996190 |
| METSMITS145B_0115 | hypothetical protein | 1003.7 | 292.8 | −3.43 | 0.9996127 |
| METSMITS145B_0190 | Ribosomal protein L7Ae/L30e/S12e/Gadd4 | 2003.0 | 486.0 | −4.12 | 0.9996116 |
| METSMITS145B_1613 | CDC6, C terminal | 282.9 | 105.3 | −2.69 | 0.9996114 |
| METSMITS145B_0477 | hypothetical protein | 251.0 | 99.3 | −2.53 | 0.9995923 |
| METSMITS145B_0734 | eIF-6 family | 1958.1 | 526.3 | −3.72 | 0.9995904 |
| METSMITS145B_1291 | hypothetical protein | 1348.3 | 4333.5 | 3.21 | 0.9995674 |
| METSMITS145B_1267 | Topoisomerase VI B subunit, transducer | 312.3 | 139.2 | −2.24 | 0.9995495 |
| METSMITS145B_0854 | Ribosomal protein S9/S16 | 758.5 | 180.4 | −4.20 | 0.9995292 |
| METSMITS145B_0581 | PhoU domain | 637.5 | 75.6 | −8.43 | 0.9995225 |
| METSMITS145B_1458 | Ribosomal protein L44 | 1860.0 | 750.1 | −2.48 | 0.9995215 |
| METSMITS145B_0647 | KOW motif | 3352.1 | 996.4 | −3.36 | 0.9995023 |
| METSMITS145B_0656 | Domain of unknown function (DUF1867) | 666.4 | 1352.6 | 2.03 | 0.9994987 |
| METSMITS145B_1359 | Proteasome A-type and B-type | 1175.0 | 339.2 | −3.46 | 0.9994870 |
| METSMITS145B_0859 | S4 domain | 913.7 | 172.1 | −5.31 | 0.9994849 |
| METSMITS145B_0692 | Ribosomal S3Ae family | 3724.4 | 1025.3 | −3.63 | 0.9994778 |
| METSMITS145B_1434 | DnaJ C terminal region | 606.2 | 119.8 | −5.06 | 0.9994702 |
| METSMITS145B_1685 | hypothetical protein | 461.3 | 210.2 | −2.19 | 0.9994676 |
| METSMITS145B_1661 | Periplasmic binding protein | 154.4 | 366.7 | 2.38 | 0.9994386 |
| METSMITS145B_1631 | hypothetical protein | 1793.0 | 754.3 | −2.38 | 0.9994340 |
| METSMITS145B_1120 | hypothetical protein | 123.6 | 398.8 | 3.23 | 0.9994298 |
| METSMITS145B_1455 | Nucleolar RNA-binding protein, Nop10p | 366.9 | 65.6 | −5.59 | 0.9994282 |
| | family | | | | |
| METSMITS145B_1835 | Uncharacterized conserved protein | 846.0 | 238.5 | −3.55 | 0.9994112 |
| | (DUF2149) | | | | |
| METSMITS145B_0843 | Polyprenyl synthetase | 303.9 | 124.2 | −2.45 | 0.9994048 |
| METSMITS145B_0711 | hypothetical protein | 86.5 | 391.4 | 4.52 | 0.9994019 |
| METSMITS145B_0275 | adhesin-like protein (Cluster 317) | 252.8 | 976.3 | 3.86 | 0.9993871 |
| METSMITS145B_0900 | Ribosomal protein L4/L1 family | 559.6 | 135.5 | −4.13 | 0.9993812 |
| METSMITS145B_0817 | Adenylosuccinate synthetase | 758.6 | 197.3 | −3.84 | 0.9993764 |
| METSMITS145B_0054 | hypothetical protein | 411.8 | 157.2 | −2.62 | 0.9993431 |
| METSMITS145B_1531 | Glycoprotease family | 208.9 | 431.2 | 2.06 | 0.9993307 |
| METSMITS145B_0415 | Phosphoribosyl transferase domain | 763.7 | 174.7 | −4.37 | 0.9993204 |
| METSMITS145B_0228 | 3′ exoribonuclease family, domain 1 | 618.2 | 209.4 | −2.95 | 0.9992944 |
| METSMITS145B_1749 | Ribosomal L37ae protein family | 2003.5 | 944.9 | −2.12 | 0.9992646 |
| METSMITS145B_0896 | Ribosomal protein L22p/L17e | 1031.7 | 317.7 | −3.25 | 0.9992573 |
| METSMITS145B_1585 | tRNA synthetases class II (D, K and N) | 489.8 | 178.9 | −2.74 | 0.9992398 |
| METSMITS145B_1060 | Staphylococcal nuclease homologue | 773.1 | 1570.0 | 2.03 | 0.9992376 |
| METSMITS145B_0898 | Ribosomal Proteins L2, C-terminal doma | 953.7 | 279.8 | −3.41 | 0.9992289 |
| METSMITS145B_1584 | HI0933-like protein | 73.6 | 23.7 | −3.11 | 0.9992283 |
| METSMITS145B_1307 | ABC transporter | 65.7 | 15.2 | −4.31 | 0.9992127 |
| METSMITS145B_0829 | Aminotransferase class I and II | 575.1 | 266.6 | −2.16 | 0.9992114 |
| METSMITS145B_0844 | Metallo-beta-lactamase superfamily | 268.7 | 66.2 | −4.06 | 0.9991340 |
| METSMITS145B_0189 | Ribosomal protein S28e | 1819.2 | 324.7 | −5.60 | 0.9991304 |
| METSMITS145B_0178 | Ribosomal protein S24e | 1310.5 | 545.1 | −2.40 | 0.9991251 |
| METSMITS145B_0199 | tRNA synthetases class I (W and Y) | 340.6 | 113.2 | −3.01 | 0.9991108 |
| METSMITS145B_0204 | TCP-1/cpn60 chaperonin family | 1542.1 | 483.0 | −3.19 | 0.9990865 |
| METSMITS145B_0732 | Prefoldin subunit | 1034.8 | 453.6 | −2.28 | 0.9990781 |
| METSMITS145B_1352 | hypothetical protein | 1820.2 | 541.5 | −3.36 | 0.9990488 |
| METSMITS145B_0505 | Universal stress protein family | 3104.8 | 7598.8 | 2.45 | 0.9990446 |
| METSMITS145B_1238 | FKBP-type peptidyl-prolyl cis-trans | 902.4 | 213.9 | −4.22 | 0.9990183 |
| | isomeras | | | | |
| METSMITS145B_0014 | hypothetical protein | 286.4 | 808.0 | 2.82 | 0.9990161 |
| METSMITS145B_0356 | PET112 family, N terminal region | 291.8 | 106.4 | −2.74 | 0.9990137 |
| METSMITS145B_1113 | hypothetical protein | 712.6 | 291.6 | −2.44 | 0.9989838 |
| METSMITS145B_1143 | MoeA N-terminal region (domain I and II | 493.6 | 191.3 | −2.58 | 0.9989256 |
| METSMITS145B_0386 | GXGXG motif | 914.6 | 223.2 | −4.10 | 0.9989225 |
| METSMITS145B_1748 | IMP dehydrogenase/GMP reductase | 769.7 | 339.6 | −2.27 | 0.9989211 |
| | domain | | | | |
| METSMITS145B_1736 | Cobalt uptake substrate-specific | 3330.3 | 912.2 | −3.65 | 0.9989004 |
| | transmembra | | | | |
| METSMITS145B_0888 | Ribosomal family S4e | 313.8 | 77.7 | −4.04 | 0.9988336 |
| METSMITS145B_1818 | FAD binding domain | 1018.4 | 482.3 | −2.11 | 0.9988300 |
| METSMITS145B_0506 | Amidohydrolase family | 193.8 | 399.2 | 2.06 | 0.9988200 |
| METSMITS145B_0968 | PRC-barrel domain | 1888.1 | 4744.9 | 2.51 | 0.9988084 |
| METSMITS145B_1141 | tRNA synthetases class I (I, L, M and V) | 304.8 | 146.7 | −2.08 | 0.9987920 |
| METSMITS145B_0933 | CBS domain pair | 1036.5 | 2349.7 | 2.27 | 0.9987890 |
| METSMITS145B_1093 | adhesin-like protein (Cluster 222) | 757.7 | 312.1 | −2.43 | 0.9987764 |
| METSMITS145B_1360 | Metallo-beta-lactamase superfamily | 142.6 | 42.4 | −3.36 | 0.9987727 |
| METSMITS145B_1174 | RNA polymerase Rpb4 | 1196.9 | 381.6 | −3.14 | 0.9987565 |
| METSMITS145B_0144 | 2,3-bisphosphoglycerate-independent pho | 707.8 | 319.3 | −2.22 | 0.9987488 |
| METSMITS145B_1660 | FdhD/NarQ family | 72.9 | 187.5 | 2.57 | 0.9987478 |
| METSMITS145B_0290 | CAAX amino terminal protease family | 935.2 | 448.3 | −2.09 | 0.9987370 |
| METSMITS145B_0106 | hypothetical protein | 940.3 | 466.0 | −2.02 | 0.9987099 |
| METSMITS145B_1602 | Amidase | 339.2 | 162.6 | −2.09 | 0.9986677 |
| METSMITS145B_0764 | Domain of unknown function DUF128 | 545.8 | 161.1 | −3.39 | 0.9986243 |
| METSMITS145B_0534 | NMD3 family | 326.3 | 129.9 | −2.51 | 0.9985967 |
| METSMITS145B_1656 | ThiC family | 5845.1 | 2037.2 | −2.87 | 0.9985545 |
| METSMITS145B_1262 | MoeA N-terminal region (domain I and II | 191.0 | 84.3 | −2.27 | 0.9985525 |
| METSMITS145B_0217 | hypothetical protein | 190.8 | 1685.3 | 8.83 | 0.9985134 |
| METSMITS145B_1204 | Ribosomal protein S12 | 930.4 | 310.1 | −3.00 | 0.9984510 |
| METSMITS145B_1738 | Cobalt transport protein | 276.5 | 79.6 | −3.48 | 0.9984390 |
| METSMITS145B_0544 | Peptidase family U32 | 172.8 | 84.6 | −2.04 | 0.9984250 |
| METSMITS145B_0832 | hypothetical protein | 261.7 | 64.1 | −4.08 | 0.9984085 |
| METSMITS145B_0894 | Ribosomal L29 protein | 433.4 | 92.5 | −4.68 | 0.9984004 |
| METSMITS145B_0887 | ribosomal L5P family C-terminus | 436.3 | 99.8 | −4.37 | 0.9983973 |
| METSMITS145B_1249 | Conserved carboxylase domain | 1286.5 | 569.9 | −2.26 | 0.9983772 |
| METSMITS145B_1123 | ABC-2 type transporter | 253.3 | 118.2 | −2.14 | 0.9983630 |
| METSMITS145B_0070 | Cysteine-rich domain | 211.0 | 457.7 | 2.17 | 0.9983307 |
| METSMITS145B_0738 | Double-stranded DNA-binding domain | 2332.3 | 1120.4 | −2.08 | 0.9983227 |
| METSMITS145B_0166 | LSM domain | 2243.6 | 712.9 | −3.15 | 0.9983088 |
| METSMITS145B_0177 | Ribosomal protein S27a | 732.1 | 281.9 | −2.60 | 0.9983070 |
| METSMITS145B_0183 | hypothetical protein | 193.9 | 34.7 | −5.59 | 0.9982994 |
| METSMITS145B_0892 | Domain of unknown function UPF0086 | 444.7 | 65.1 | −6.83 | 0.9982397 |
| METSMITS145B_0741 | RNAse P Rpr2/Rpp21/SNM1 subunit | 2696.9 | 777.6 | −3.47 | 0.9982195 |
| | domain | | | | |
| METSMITS145B_0154 | adhesin-like protein (Cluster 92) | 55.7 | 120.1 | 2.16 | 0.9982156 |
| METSMITS145B_0595 | NIF3 (NGG1p interacting factor 3) | 153.5 | 47.2 | −3.25 | 0.9982128 |
| METSMITS145B_0747 | DNA topoisomerase | 235.6 | 107.2 | −2.20 | 0.9981900 |
| METSMITS145B_0406 | ACT domain | 363.3 | 771.6 | 2.12 | 0.9981515 |
| METSMITS145B_0503 | hypothetical protein | 347.7 | 142.0 | −2.45 | 0.9981511 |
| METSMITS145B_0875 | Integral membrane protein DUF106 | 726.1 | 150.6 | −4.82 | 0.9981432 |
| METSMITS145B_0740 | CRS1/YhbY (CRM) domain | 2241.9 | 303.2 | −7.40 | 0.9980914 |
| METSMITS145B_0035 | 2′,5′ RNA ligase family | 229.0 | 87.6 | −2.61 | 0.9979889 |
| METSMITS145B_0268 | hypothetical protein | 36.2 | 103.1 | 2.85 | 0.9979688 |
| METSMITS145B_0179 | Protein of unknown function (DUF359) | 952.4 | 388.4 | −2.45 | 0.9979441 |
| METSMITS145B_0883 | Ribosomal protein L32 | 477.7 | 120.5 | −3.97 | 0.9979379 |
| METSMITS145B_0884 | Ribosomal protein L6 | 448.3 | 125.7 | −3.57 | 0.9979229 |
| METSMITS145B_1457 | Ribosomal protein S27 | 1013.8 | 378.9 | −2.68 | 0.9977081 |
| METSMITS145B_0980 | hypothetical protein | 2545.5 | 1171.2 | −2.17 | 0.9976843 |
| METSMITS145B_1092 | hypothetical protein | 658.7 | 278.5 | −2.37 | 0.9976820 |
| METSMITS145B_1163 | 8-oxoguanine DNA glycosylase, N-terminal | 80.8 | 31.3 | −2.58 | 0.9976816 |
| | dom | | | | |
| METSMITS145B_1518 | Nitrogen regulatory protein P-II | 1068.9 | 139.1 | −7.69 | 0.9976814 |
| METSMITS145B_0220 | Nitrogen regulatory protein P-II | 1068.9 | 139.1 | −7.69 | 0.9976814 |
| METSMITS145B_1850 | Rubrerythrin | 4998.3 | 14204.1 | 2.84 | 0.9975017 |
| METSMITS145B_1313 | hypothetical protein | 70.1 | 19.9 | −3.52 | 0.9974803 |
| METSMITS145B_1430 | GrpE | 141.5 | 33.7 | −4.19 | 0.9974753 |
| METSMITS145B_0579 | 4Fe—4S binding domain | 194.2 | 59.6 | −3.26 | 0.9974621 |
| METSMITS145B_0624 | hypothetical protein | 276.1 | 96.5 | −2.86 | 0.9973842 |
| METSMITS145B_0380 | hypothetical protein | 50.7 | 23.7 | −2.14 | 0.9973443 |
| METSMITS145B_0181 | RNA polymerase Rpb7-like, N-terminal d | 1063.1 | 433.0 | −2.46 | 0.9973353 |
| METSMITS145B_0889 | KOW motif | 942.2 | 281.6 | −3.35 | 0.9971608 |
| METSMITS145B_0423 | hypothetical protein | 681.3 | 320.1 | −2.13 | 0.9970890 |
| METSMITS145B_1565 | hypothetical protein | 423.6 | 865.4 | 2.04 | 0.9970848 |
| METSMITS145B_0288 | ABC transporter | 268.3 | 126.6 | −2.12 | 0.9970833 |
| METSMITS145B_0877 | eubacterial secY protein | 1062.7 | 445.0 | −2.39 | 0.9970693 |
| METSMITS145B_1431 | Hsp70 protein | 642.9 | 256.0 | −2.51 | 0.9969834 |
| METSMITS145B_1605 | 3,4-dihydroxy-2-butanone 4-phosphate sy | 554.2 | 202.0 | −2.74 | 0.9968535 |
| METSMITS145B_1410 | Sir2 family | 284.3 | 121.8 | −2.33 | 0.9968306 |
| METSMITS145B_0760 | S-adenosyl-L-homocysteine hydrolase, NA | 854.7 | 368.6 | −2.32 | 0.9967602 |
| METSMITS145B_0059 | hypothetical protein | 386.7 | 167.5 | −2.31 | 0.9967564 |
| METSMITS145B_1444 | Hydrogenase maturation protease | 3568.0 | 7435.4 | 2.08 | 0.9966401 |
| METSMITS145B_1096 | Chlamydia polymorphic membrane protein | 98.6 | 31.9 | −3.09 | 0.9966163 |
| | (Chl | | | | |
| METSMITS145B_0856 | Ribosomal protein L15 | 858.0 | 178.6 | −4.80 | 0.9965206 |
| METSMITS145B_0848 | Memo-like protein | 132.2 | 45.9 | −2.88 | 0.9964448 |
| METSMITS145B_0872 | hypothetical protein | 2094.6 | 460.3 | −4.55 | 0.9963996 |
| METSMITS145B_0949 | ABC transporter | 415.5 | 156.6 | −2.65 | 0.9963777 |
| METSMITS145B_0427 | hypothetical protein | 520.9 | 161.3 | −3.23 | 0.9963302 |
| METSMITS145B_1302 | Pyridoxal-dependent decarboxylase conse | 136.5 | 37.9 | −3.60 | 0.9962536 |
| METSMITS145B_1538 | methylene-5,6,7,8- | 8996.2 | 24598.8 | 2.73 | 0.9962188 |
| | tetrahydromethanopterin de | | | | |
| METSMITS145B_0576 | Pyruvate flavodoxin/ferredoxin oxidor | 851.8 | 401.3 | −2.12 | 0.9958999 |
| METSMITS145B_1496 | hypothetical protein | 438.9 | 1271.5 | 2.90 | 0.9958583 |
| METSMITS145B_1222 | Thiamine monophosphate synthase/TENI | 152.4 | 50.0 | −3.05 | 0.9957322 |
| METSMITS145B_0052 | hypothetical protein | 184.2 | 546.8 | 2.97 | 0.9957191 |
| METSMITS145B_0948 | Initiation factor 2 subunit family | 476.7 | 221.5 | −2.15 | 0.9956836 |
| METSMITS145B_0964 | Domain of unknown function (DUF1724) | 37.1 | 148.0 | 3.99 | 0.9954538 |
| METSMITS145B_1739 | ABC transporter | 181.6 | 52.0 | −3.49 | 0.9954323 |
| METSMITS145B_1136 | Serine hydroxymethyltransferase | 720.1 | 334.8 | −2.15 | 0.9953723 |
| METSMITS145B_0497 | hypothetical protein | 185.6 | 88.8 | −2.09 | 0.9953688 |
| METSMITS145B_1308 | Substrate binding domain of ABC-type gly | 52.1 | 13.4 | −3.90 | 0.9950982 |
| METSMITS145B_0623 | hypothetical protein | 5695.6 | 1596.0 | −3.57 | 0.9950323 |
| METSMITS145B_1481 | tRNA pseudouridine synthase D (TruD) | 203.1 | 100.0 | −2.03 | 0.9949935 |
| METSMITS145B_1750 | Brix domain | 264.3 | 78.2 | −3.38 | 0.9949691 |
| METSMITS145B_0062 | Thymidylate kinase | 328.9 | 136.9 | −2.40 | 0.9949192 |
| METSMITS145B_0266 | Anticodon-binding domain | 554.3 | 247.9 | −2.24 | 0.9948838 |
| METSMITS145B_0028 | NADP oxidoreductase coenzyme F420- | 732.8 | 1959.3 | 2.67 | 0.9948097 |
| | depe | | | | |
| METSMITS145B_1182 | hypothetical protein | 465.3 | 141.4 | −3.29 | 0.9947834 |
| METSMITS145B_0535 | tRNA synthetases class I (W and Y) | 385.6 | 142.9 | −2.70 | 0.9946715 |
| METSMITS145B_0164 | hypothetical protein | 528.8 | 1108.3 | 2.10 | 0.9946358 |
| METSMITS145B_0853 | RNA polymerases N/8 kDa subunit | 239.1 | 80.0 | −2.99 | 0.9945515 |
| METSMITS145B_1321 | hypothetical protein | 370.5 | 174.2 | −2.13 | 0.9944052 |
| METSMITS145B_1177 | Zinc-binding dehydrogenase | 1040.7 | 6448.2 | 6.20 | 0.9943331 |
| METSMITS145B_0622 | EF-1 guanine nucleotide exchange domain | 641.0 | 252.5 | −2.54 | 0.9942381 |
| METSMITS145B_0660 | PUA domain | 447.6 | 203.6 | −2.20 | 0.9940320 |
| METSMITS145B_0349 | hypothetical protein | 131.9 | 44.8 | −2.94 | 0.9936418 |
| METSMITS145B_0879 | Ribosomal protein L30p/L7e | 559.9 | 206.9 | −2.71 | 0.9935049 |
| METSMITS145B_0063 | hypothetical protein | 359.4 | 133.5 | −2.69 | 0.9934970 |
| METSMITS145B_0583 | hypothetical protein | 1081.9 | 205.1 | −5.27 | 0.9933050 |
| METSMITS145B_1269 | Type IIB DNA topoisomerase | 559.3 | 215.7 | −2.59 | 0.9932221 |
| METSMITS145B_0609 | Ferrous iron transport protein B | 133.3 | 315.1 | 2.36 | 0.9928589 |
| METSMITS145B_0897 | Ribosomal protein S19 | 815.1 | 325.5 | −2.50 | 0.9926403 |
| METSMITS145B_1219 | Tetratricopeptide repeat | 12.9 | 43.1 | 3.33 | 0.9924422 |
| METSMITS145B_1394 | NADH-Ubiquinone/plastoquinone (complex | 273.2 | 123.0 | −2.22 | 0.9923872 |
| | I) | | | | |
| METSMITS145B_0022 | Aminotransferase class I and II | 71.4 | 24.3 | −2.94 | 0.9922219 |
| METSMITS145B_0831 | hypothetical protein | 203.4 | 95.8 | −2.12 | 0.9921651 |
| METSMITS145B_0578 | 4Fe—4S binding domain | 869.8 | 397.6 | −2.19 | 0.9921494 |
| METSMITS145B_0735 | Ribosomal protein L31e | 1124.2 | 429.2 | −2.62 | 0.9918193 |
| METSMITS145B_0893 | Translation initiation factor SUI1 | 280.1 | 74.6 | −3.76 | 0.9917441 |
| METSMITS145B_0034 | adhesin-like protein (Cluster 18) | 245.6 | 96.6 | −2.54 | 0.9917377 |
| METSMITS145B_1114 | hypothetical protein | 315.2 | 104.0 | −3.03 | 0.9916374 |
| METSMITS145B_1432 | hypothetical protein | 455.9 | 128.2 | −3.56 | 0.9916028 |
| METSMITS145B_0322 | ABC transporter | 328.2 | 138.0 | −2.38 | 0.9913972 |
| METSMITS145B_0447 | Toprim domain | 498.8 | 245.5 | −2.03 | 0.9909964 |
| METSMITS145B_1825 | hypothetical protein | 71.8 | 27.3 | −2.62 | 0.9909923 |
| METSMITS145B_0749 | hypothetical protein | 475.5 | 158.5 | −3.00 | 0.9907966 |
| METSMITS145B_1356 | YLP motif | 662.5 | 255.6 | −2.59 | 0.9907192 |
| METSMITS145B_1002 | hypothetical protein | 2145.8 | 5907.5 | 2.75 | 0.9903513 |
| METSMITS145B_0524 | hypothetical protein | 92.0 | 200.6 | 2.18 | 0.9901892 |
| METSMITS145B_0246 | hypothetical protein | 68.8 | 27.3 | −2.52 | 0.9899688 |
| METSMITS145B_0891 | Ribosomal protein S17 | 499.7 | 207.6 | −2.41 | 0.9898280 |
| METSMITS145B_0885 | Ribosomal protein S8 | 550.5 | 208.5 | −2.64 | 0.9898115 |
| METSMITS145B_0881 | Ribosomal L18p/L5e family | 841.1 | 349.9 | −2.40 | 0.9896522 |
| METSMITS145B_1752 | Prefoldin subunit | 604.8 | 280.1 | −2.16 | 0.9895336 |
| METSMITS145B_0850 | 4Fe—4S binding domain | 252.8 | 46.8 | −5.40 | 0.9895237 |
| METSMITS145B_1678 | hypothetical protein | 82.5 | 40.2 | −2.06 | 0.9891309 |
| METSMITS145B_1218 | RNA polymerase Rpb5, C-terminal domain | 463.5 | 212.8 | −2.18 | 0.9886263 |
| METSMITS145B_0537 | hypothetical protein | 504.4 | 1372.1 | 2.72 | 0.9884802 |
| METSMITS145B_0230 | KH domain | 244.8 | 95.3 | −2.57 | 0.9884130 |
| METSMITS145B_0564 | Cytidylyltransferase | 48.4 | 21.3 | −2.27 | 0.9882508 |
| METSMITS145B_1517 | Ammonium Transporter Family | 1021.8 | 93.0 | −10.98 | 0.9879685 |
| METSMITS145B_0221 | Ammonium Transporter Family | 1021.8 | 93.0 | −10.98 | 0.9879685 |
| METSMITS145B_1029 | Sodium: neurotransmitter symporter family | 94.0 | 31.1 | −3.03 | 0.9878348 |
| METSMITS145B_0804 | DNA polymerase family B | 158.2 | 352.3 | 2.23 | 0.9876538 |
| METSMITS145B_0599 | adhesin-like protein (Cluster 1267) | 191.4 | 90.4 | −2.12 | 0.9874649 |
| METSMITS145B_0890 | Ribosomal protein L14p/L23e | 536.9 | 185.0 | −2.90 | 0.9874616 |
| METSMITS145B_1686 | hypothetical protein | 342.5 | 88.7 | −3.86 | 0.9873751 |
| METSMITS145B_1039 | Methyltransferase domain | 288.9 | 130.6 | −2.21 | 0.9873505 |
| METSMITS145B_0143 | MatE | 71.6 | 23.9 | −3.00 | 0.9869973 |
| METSMITS145B_0874 | Integral membrane protein DUF106 | 330.0 | 113.0 | −2.92 | 0.9869525 |
| METSMITS145B_1547 | Peptidase family M48 | 75.1 | 162.2 | 2.16 | 0.9866127 |
| METSMITS145B_0036 | 3-dehydroquinate synthase (EC 4.6.1.3) | 400.8 | 199.6 | −2.01 | 0.9865625 |
| METSMITS145B_0367 | Protein of unknown function (DUF509) | 221.5 | 100.5 | −2.20 | 0.9865053 |
| METSMITS145B_0878 | Ribosomal protein L15 | 516.5 | 237.4 | −2.18 | 0.9864541 |
| METSMITS145B_0600 | Protein of unknown function DUF70 | 109.3 | 31.0 | −3.53 | 0.9864365 |
| METSMITS145B_1537 | Peptidase family M48 | 65.8 | 140.4 | 2.13 | 0.9862686 |
| METSMITS145B_0536 | hypothetical protein | 691.9 | 316.8 | −2.18 | 0.9862085 |
| METSMITS145B_0196 | Histone-like transcription factor (CBF/ | 116600.0 | 233350.9 | 2.00 | 0.9860558 |
| METSMITS145B_1804 | hypothetical protein | 6.5 | 14.7 | 2.26 | 0.9858840 |
| METSMITS145B_0838 | Cupin domain | 989.7 | 2032.4 | 2.05 | 0.9858283 |
| METSMITS145B_0707 | hypothetical protein | 54.4 | 19.0 | −2.86 | 0.9848908 |
| METSMITS145B_1834 | hypothetical protein | 378.5 | 124.5 | −3.04 | 0.9847138 |
| METSMITS145B_1666 | hypothetical protein | 69.3 | 27.5 | −2.52 | 0.9838153 |
| METSMITS145B_0827 | hypothetical protein | 265.7 | 111.7 | −2.38 | 0.9837558 |
| METSMITS145B_0994 | hypothetical protein | 296.2 | 121.7 | −2.43 | 0.9836185 |
| METSMITS145B_0826 | hypothetical protein | 76.9 | 23.6 | −3.26 | 0.9830315 |
| METSMITS145B_0538 | B12 binding domain | 362.5 | 1019.1 | 2.81 | 0.9828607 |
| METSMITS145B_0357 | hypothetical protein | 111.7 | 245.7 | 2.20 | 0.9821259 |
| METSMITS145B_1424 | Phosphoribosyl-ATP | 447.5 | 208.0 | −2.15 | 0.9820992 |
| | pyrophosphohydrolase | | | | |
| METSMITS145B_1281 | Shikimate/quinate 5-dehydrogenase | 126.8 | 58.6 | −2.16 | 0.9820247 |
| METSMITS145B_0632 | yrdC domain | 106.0 | 44.9 | −2.36 | 0.9811305 |
| METSMITS145B_0899 | Ribosomal protein L23 | 524.8 | 190.1 | −2.76 | 0.9809105 |
| METSMITS145B_0396 | hypothetical protein | 4585.6 | 332.4 | −13.80 | 0.9804666 |
| METSMITS145B_0105 | hypothetical protein | 155.8 | 29.4 | −5.30 | 0.9802722 |
| METSMITS145B_1663 | ABC transporter | 75.6 | 163.2 | 2.16 | 0.9788921 |
| METSMITS145B_0963 | hypothetical protein | 68.3 | 230.8 | 3.38 | 0.9781729 |
| METSMITS145B_1346 | Sodium/calcium exchanger protein | 148.2 | 66.5 | −2.23 | 0.9779362 |
| METSMITS145B_0300 | hypothetical protein | 90.9 | 30.7 | −2.96 | 0.9775189 |
| METSMITS145B_1030 | Sodium: neurotransmitter symporter family | 156.3 | 68.8 | −2.27 | 0.9774789 |
| METSMITS145B_1824 | 4Fe—4S iron sulfur cluster binding proteins | 141.7 | 55.0 | −2.58 | 0.9772790 |
| METSMITS145B_0464 | hypothetical protein | 255.8 | 565.0 | 2.21 | 0.9770922 |
| METSMITS145B_1670 | hypothetical protein | 26.1 | 11.1 | −2.34 | 0.9768899 |
| METSMITS145B_0434 | hypothetical protein | 24.5 | 62.1 | 2.54 | 0.9766617 |
| METSMITS145B_0276 | hypothetical protein | 158.0 | 672.7 | 4.26 | 0.9765445 |
| METSMITS145B_0381 | NikR C terminal nickel binding domain | 2712.3 | 1008.3 | −2.69 | 0.9760396 |
| METSMITS145B_0292 | hypothetical protein | 147.9 | 465.8 | 3.15 | 0.9759447 |
| METSMITS145B_0064 | hypothetical protein | 976.2 | 486.1 | −2.01 | 0.9759221 |
| METSMITS145B_1577 | MatE | 53.0 | 26.2 | −2.02 | 0.9738624 |
| METSMITS145B_0248 | hypothetical protein | 126.4 | 41.7 | −3.03 | 0.9730076 |
| METSMITS145B_0450 | hypothetical protein | 137.1 | 51.2 | −2.68 | 0.9726703 |
| METSMITS145B_0700 | Protein of unknown function DUF101 | 56.9 | 161.7 | 2.84 | 0.9724525 |
| METSMITS145B_0305 | hypothetical protein | 52.7 | 26.2 | −2.01 | 0.9721916 |
| METSMITS145B_0765 | Uncharacterized protein conserved in | 303.7 | 640.0 | 2.11 | 0.9714954 |
| | archaea | | | | |
| METSMITS145B_0662 | ACT domain | 150.3 | 45.5 | −3.30 | 0.9704155 |
| METSMITS96A_1127 | Uncharacterized protein conserved in | 459.6 | 176.9 | −2.60 | 1.0000 |
| | archaea | | | | |
| METSMITS96A_0937 | Elongation factor Tu GTP binding domain | 439.8 | 988.1 | 2.25 | 1.0000 |
| METSMITS96A_1571 | CoA binding domain | 167.2 | 844.3 | 5.05 | 0.9999 |
| METSMITS96A_0605 | 4Fe—4S binding domain | 50.1 | 287.7 | 5.75 | 0.9999 |
| METSMITS96A_0778 | Ribosomal protein L3 | 331.4 | 736.7 | 2.22 | 0.9998 |
| METSMITS96A_0026 | Acetyltransferase (GNAT) family | 2152.7 | 954.9 | −2.25 | 0.9998 |
| METSMITS96A_0075 | adhesin-like protein (Cluster 18) | 150.1 | 72.3 | −2.08 | 0.9998 |
| METSMITS96A_1071 | AsnC family | 1146.2 | 443.9 | −2.58 | 0.9998 |
| METSMITS96A_0603 | Pyruvate flavodoxin/ferredoxin oxidor | 45.1 | 251.4 | 5.57 | 0.9997 |
| METSMITS96A_1455 | adhesin-like protein (Cluster 37) | 84.5 | 30.0 | −2.82 | 0.9996 |
| METSMITS96A_0777 | Ribosomal protein L4/L1 family | 155.9 | 464.0 | 2.98 | 0.9996 |
| METSMITS96A_0593 | hypothetical protein | 974.4 | 479.5 | −2.03 | 0.9993 |
| METSMITS96A_1126 | Major intrinsic protein | 281.8 | 107.5 | −2.62 | 0.9993 |
| METSMITS96A_0604 | Thiamine pyrophosphate enzyme, C- | 53.6 | 335.9 | 6.26 | 0.9993 |
| | termina | | | | |
| METSMITS96A_0948 | RNA polymerase Rpb1, domain 2 | 128.9 | 258.2 | 2.00 | 0.9993 |
| METSMITS96A_0403 | Helix-turn-helix | 1264.1 | 597.2 | −2.12 | 0.9989 |
| METSMITS96A_0626 | Peptide methionine sulfoxide reductase | 523.4 | 230.3 | −2.27 | 0.9987 |
| METSMITS96A_1014 | hypothetical protein | 4182.6 | 1728.2 | −2.42 | 0.9986 |
| METSMITS96A_1260 | hypothetical protein | 125.6 | 46.8 | −2.68 | 0.9985 |
| METSMITS96A_0601 | Pyruvate ferredoxin/flavodoxin | 336.6 | 1075.0 | 3.19 | 0.9984 |
| | oxidoreductas | | | | |
| METSMITS96A_1456 | Chlamydia polymorphic membrane protein | 243.5 | 118.7 | −2.05 | 0.9984 |
| | (Chl | | | | |
| METSMITS96A_0239 | TCP-1/cpn60 chaperonin family | 207.4 | 422.6 | 2.04 | 0.9983 |
| METSMITS96A_0947 | RNA polymerase Rpb1, domain 5 | 656.1 | 1373.2 | 2.09 | 0.9982 |
| METSMITS96A_0913 | Glutamate/Leucine/Phenylalanine/Valin | 347.6 | 782.8 | 2.25 | 0.9980 |
| METSMITS96A_0926 | Glutamate/Leucine/Phenylalanine/Valin | 347.6 | 782.8 | 2.25 | 0.9980 |
| METSMITS96A_1542 | Sugar-specific transcriptional regulator Trm | 228.7 | 53.0 | −4.31 | 0.9977 |
| METSMITS96A_0732 | hypothetical protein | 1541.1 | 634.2 | −2.43 | 0.9975 |
| METSMITS96A_1524 | S4 domain | 326.0 | 659.6 | 2.02 | 0.9974 |
| METSMITS96A_1119 | adhesin-like protein (Cluster 226) | 673.8 | 179.8 | −3.75 | 0.9973 |
| METSMITS96A_0349 | Ribosomal L15 | 1139.3 | 2798.6 | 2.46 | 0.9969 |
| METSMITS96A_1374 | Chlamydia polymorphic membrane protein | 149.9 | 73.9 | −2.03 | 0.9961 |
| | (Chl | | | | |
| METSMITS96A_0373 | Predicted membrane protein (DUF2107) | 734.6 | 341.7 | −2.15 | 0.9957 |
| METSMITS96A_1733 | Uncharacterized conserved protein | 2328.9 | 742.3 | −3.14 | 0.9955 |
| | (DUF2304) | | | | |
| METSMITS96A_1793 | hypothetical protein | 1206.1 | 594.3 | −2.03 | 0.9954 |
| METSMITS96A_1758 | hypothetical protein | 1312.0 | 595.6 | −2.20 | 0.9954 |
| METSMITS96A_1532 | Enolase, C-terminal TIM barrel domain | 47.7 | 95.7 | 2.01 | 0.9952 |
| METSMITS96A_1849 | hypothetical protein | 269.7 | 126.4 | −2.13 | 0.9951 |
| METSMITS96A_1403 | 4Fe—4S binding domain | 2597.7 | 5467.9 | 2.10 | 0.9950 |
| METSMITS96A_0945 | KH domain | 790.6 | 1791.8 | 2.27 | 0.9950 |
| METSMITS96A_0935 | Ribosomal protein S10p/S20e | 302.4 | 868.1 | 2.87 | 0.9937 |
| METSMITS96A_1519 | Transposase DDE domain | 49.5 | 21.1 | −2.34 | 0.9934 |
| METSMITS96A_0833 | hypothetical protein | 394.4 | 133.6 | −2.95 | 0.9929 |
| METSMITS96A_0720 | hypothetical protein | 434.5 | 161.3 | −2.69 | 0.9926 |
| METSMITS96A_0087 | hypothetical protein | 43.7 | 19.0 | −2.31 | 0.9917 |
| METSMITS96A_0304 | Uncharacterised protein family UPF0047 | 388.8 | 95.0 | −4.09 | 0.9911 |
| METSMITS96A_0859 | Chlamydia polymorphic membrane protein | 209.8 | 98.3 | −2.13 | 0.9910 |
| | (Chl | | | | |
| METSMITS96A_0973 | hypothetical protein | 91.0 | 36.1 | −2.52 | 0.9900 |
| METSMITS96A_0974 | hypothetical protein | 661.1 | 284.5 | −2.32 | 0.9895 |
| METSMITS96A_0347 | Archaeal ATPase | 141.9 | 68.1 | −2.08 | 0.9893 |
| METSMITS96A_0272 | hypothetical protein | 439.7 | 216.2 | −2.03 | 0.9893 |
| METSMITS96A_0005 | hypothetical protein | 171.4 | 76.9 | −2.23 | 0.9884 |
| METSMITS96A_0664 | Ribosomal protein L11, N-terminal dom | 1444.2 | 3049.9 | 2.11 | 0.9877 |
| METSMITS96A_1347 | hypothetical protein | 496.1 | 171.7 | −2.89 | 0.9873 |
| METSMITS96A_0501 | Helix-turn-helix | 683.8 | 270.7 | −2.53 | 0.9867 |
| METSMITS96A_0919 | E1-E2 ATPase | 171.4 | 82.3 | −2.08 | 0.9862 |
| METSMITS96A_1650 | Glycosyl transferase family 2 | 105.8 | 46.5 | −2.28 | 0.9861 |
| METSMITS96A_0602 | 4Fe—4S binding domain | 57.2 | 250.6 | 4.38 | 0.9859 |
| METSMITS96A_1529 | Ribosomal protein S9/S16 | 316.2 | 679.4 | 2.15 | 0.9852 |
| METSMITS96A_0050 | hypothetical protein | 576.9 | 280.1 | −2.06 | 0.9846 |
| METSMITS96A_1591 | hypothetical protein | 59.5 | 169.9 | 2.86 | 0.9832 |
| METSMITS96A_0093 | hypothetical protein | 349.3 | 168.8 | −2.07 | 0.9826 |
| METSMITS96A_0019 | Exonuclease VII small subunit | 68.7 | 24.2 | −2.84 | 0.9824 |
| METSMITS96A_1783 | Transcription factor S-II (TFIIS) | 633.2 | 302.7 | −2.09 | 0.9820 |
| METSMITS96A_0189 | hypothetical protein | 149.7 | 73.4 | −2.04 | 0.9816 |
| METSMITS96A_1107 | Domain related to MnhB subunit of Na+/H+ | 21.7 | 51.5 | 2.37 | 0.9810 |
| | ant | | | | |
| METSMITS96A_0885 | HxlR-like helix-turn-helix | 332.2 | 126.0 | −2.64 | 0.9809 |
| METSMITS96A_1237 | 6-O-methylguanine DNA methyltransferase | 221.2 | 99.0 | −2.23 | 0.9805 |
| METSMITS96A_0253 | hypothetical protein | 412.8 | 126.2 | −3.27 | 0.9799 |
| METSMITS96A_1566 | Histidine kinase-, DNA gyrase B-, and | 75.0 | 34.0 | −2.21 | 0.9781 |
| | HSP90 | | | | |
| METSMITS96A_0852 | GHMP kinases N terminal domain | 45.2 | 91.8 | 2.03 | 0.9775 |
| METSMITS96A_0746 | RNAse P Rpr2/Rpp21/SNM1 subunit | 707.8 | 1480.0 | 2.09 | 0.9769 |
| | domain | | | | |
| METSMITS96A_1611 | hypothetical protein | 57.6 | 25.1 | −2.30 | 0.9765 |
| METSMITS96A_1628 | hypothetical protein | 44.4 | 21.8 | −2.04 | 0.9764 |
| METSMITS96A_1064 | Domain of unknown function (DUF1922) | 4893.8 | 2191.7 | −2.23 | 0.9764 |
| METSMITS96A_0765 | Ribosomal family S4e | 101.0 | 214.3 | 2.12 | 0.9750 |
| METSMITS96A_0116 | NADP oxidoreductase coenzyme F420- | 38.1 | 17.3 | −2.20 | 0.9744 |
| | depe | | | | |
| METSMITS96A_1102 | hypothetical protein | 25.6 | 57.4 | 2.25 | 0.9735 |
| METSMITS96A_1559 | Coenzyme F420 | 37.4 | 16.6 | −2.26 | 0.9733 |
| | hydrogenase/dehydrogenase, | | | | |
| METSMITS96A_1822 | YLP motif | 34.3 | 69.0 | 2.01 | 0.9715 |
| METSMITS96A_0301 | hypothetical protein | 844.2 | 361.9 | −2.33 | 0.9714 |
| METSMITS96A_0061 | hypothetical protein | 2255.9 | 803.8 | −2.81 | 0.9709 |

Genes significantly regulated by formate were identified for each strain by analyzing normalized reads by CyberT, whch calculates a posterior probability of differential expression (PPDE) statistic to determine significance (PPDE ≧ 0.97 and at least a twofold difference between coditions).

### Example 11

### Horizontal Gene Transfer (HG)

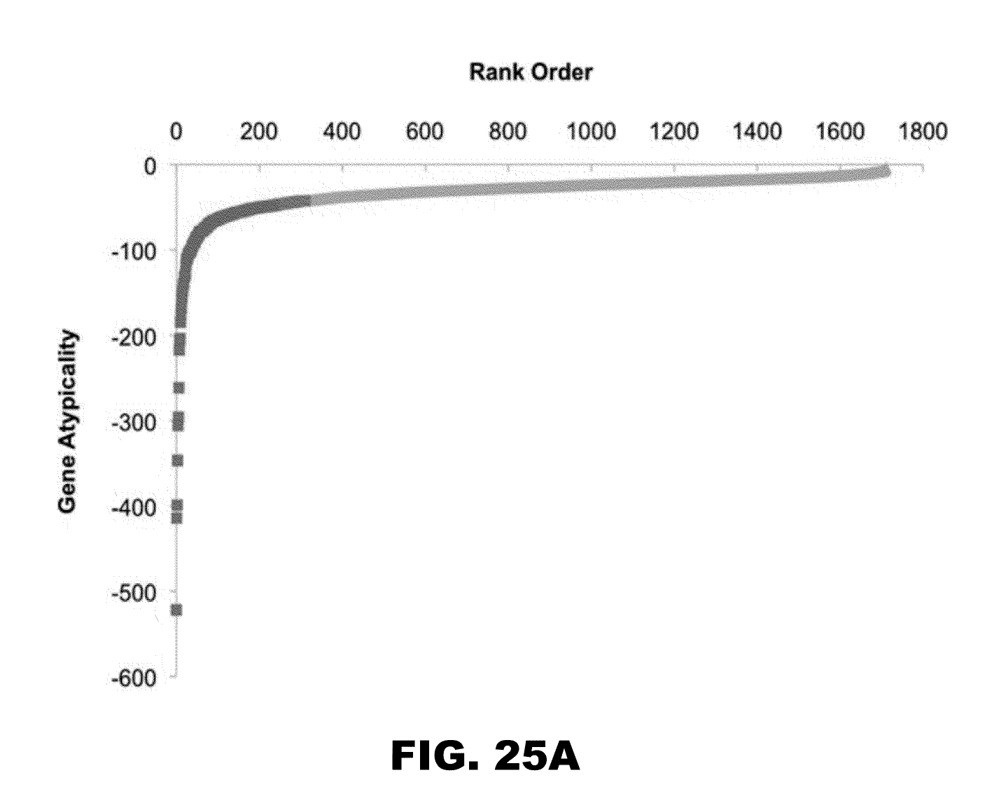

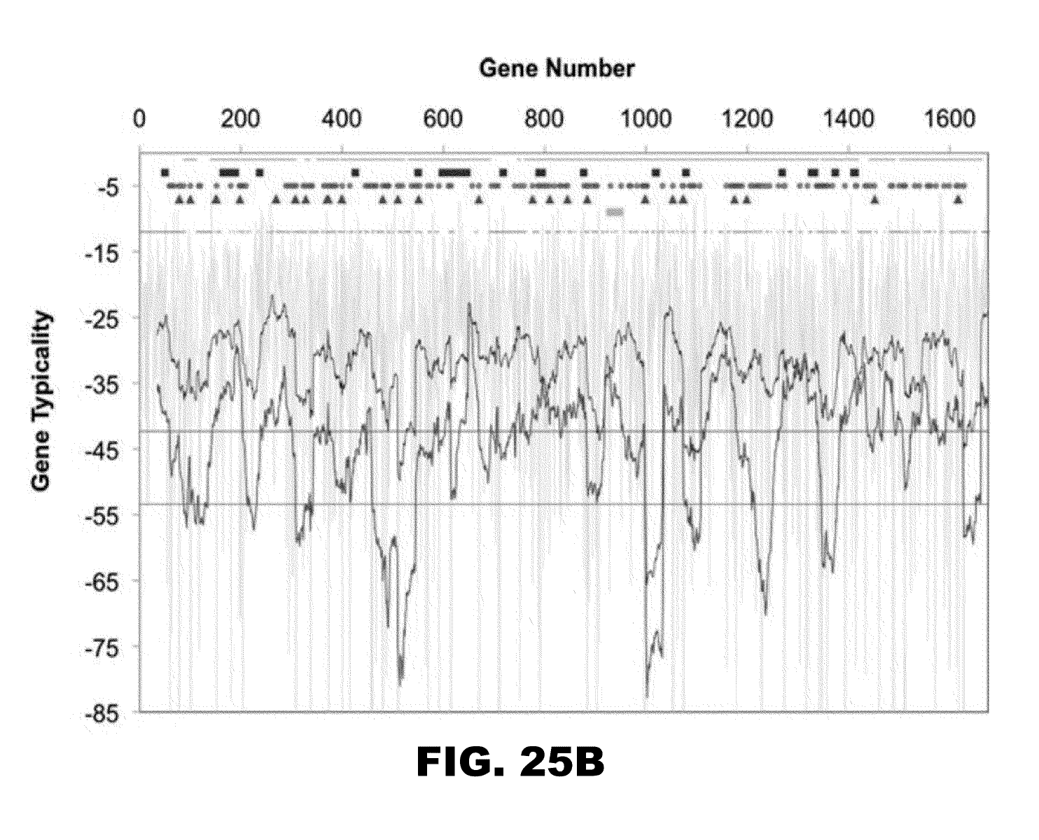

To better understand genomic differences among M. smithii strains, HGT was detected by using both compositional and phylogenetic methods. Compositional HGT detection was performed by examining the typicality of dinucleotides, codons, and k-words of lengths 4 and 6. Because highly expressed genes are known to contain unusual compositions, genes were scored for typicality against both a whole-genome compositional model and a model built using ribosomal proteins (55, 56). Only genes found to be below the significance threshold when compared against both models were annotated as transferred. To select significance thresholds for transfer, genes in each genome were ordered from most to least atypical. As reported (57), gene typicality was observed to increase rapidly for the most extreme genes, and then to rise only gradually for the rest of the genome (FIG. 25A). In this case, thresholds were set at the point where the change among the overlapping 30 gene windows was <0.1% of the score of the previous window.

Among the compositional measures analyzed, the proportion of genes defined as horizontally transferred ranged from 3.3 to 10.1% in the dataset as a whole. However, because the absolute number of horizontally transferred genes predicted can depend on the compositional measure chosen, the stringency of the thresholds selected, the amount of time that has passed since the transfer occurred, and the compositional distinctiveness of gene transfer donors (ref. 58; reviewed in ref. 56), this analysis did not focus on the absolute magnitude of gene transfer in these lineages. Instead, differences in the frequency of HGT events for different classes of genes were of primary interest, in addition to how this process has contributed to the evolution and specialization of the characterized M. smithii strains.

When using compositional methods, it was observed that gene transfer is more frequent in the variable genome than the core. For example, when examining 3-1 dinucleotide use (55) and using the rank order of G scores as the significance threshold, 5.7% of the core genes in the pan-genome show compositional evidence of transfer, compared with fully 16.4% of the variably represented genes, suggesting an approximately threefold enrichment of gene transfer in the variable relative to the core components of the pan-genome.

However, others have observed that phylogenetic methods tend to detect more ancient transfer events than compositional methods (59). Consistent with these observations, 73% of the genes for which PhyloNet found evidence of HGT were part of M. smithii's core genome, indicating transfer before the divergence of strains. By contrast, most putative HGT events predicted by compositional methods were part of the variable genome (59.3-68.0% of transfers, depending on the method) (Tables 20 and 21). This difference may be due in part to the requirement of phylogenetic methods for orthologs of the gene under investigation: Compositional HGT predictions for the subset of genes that could be mapped to KEGG orthology groups were also biased toward the core genome. Genes with both compositional and phylogenetic evidence of transfer tend to be more evenly split between the core and variable genomes than transfers supported by either type of evidence alone (Tables 20 and 21).

Taken together, these findings suggest that gene transfer has shaped both the core genome of M. smithii and differences between strains. External evidence further supports a role for HGT in shaping the core genome of M. smithii: 89.1% of genes within prophage (as detected by PhageFinder) are part of the core genome (Tables 20 and 21).

### Functional Contribution of Horizontally Transferred Genes.

To test for differences in the functions contributed to the M. smithii pan-genome by the core genome, variable genome, or horizontally transferred genes, each of these three gene sets were annotated to KEGG pathways (level 2). The M. smithii core genome is enriched in genes involved in “translation” while being depleted in “membrane transporters” and “unclassified metabolic” genes (Bonferroni-corrected G-test for significance; P<0.001). The variable genome is enriched in genes for membrane transporters, “glycan biosynthesis and metabolism,” and genes whose functions are poorly characterized, while being depleted for genes involved in translation (Bonferroni-corrected G-test; P<0.001). Horizontally transferred genes, regardless of the detection method used, are most divergent from the pan-genome in their functional profile than either the core or variable components of the M. smithii pan-genome. This finding suggests that gene transfer has contributed significant functional diversity to M. smithii.

To understand in more detail the specific categories of genes that have been most frequently transferred, significant HGT results for 3-1 dinucleotide use were pooled across genomes and categorized according to KEGG pathway and KEGG orthology group, weighting genes with multiple pathway annotations on a per gene (rather than per annotation) basis (Table 32). As previously observed for genomic islands (60), genes of unknown or poorly characterized function dominated the HGT pool. Among genes with known KEGG level 2 pathway annotations, those in the KEGG category for folate biosynthesis were the most frequently transferred (101.7 normalized annotations). Tetrahydromethanopterin (THMP) methyltransferase genes were the most frequently transferred KEGG orthology (KO) within this group (23 putative HGT events for the D subunit). THMP methylransferase (61) participates in both the methanogenesis and folate biosynthesis pathways by transferring a methyl group from 5-Methyl-THMP to coenzyme-M (FIG. 24). Genes involved in coenzyme-M recycling during methanogenesis were similarly frequently transferred, including methyl-coenzyme M reductase α subunit (EC 2.8.4.1; 23 annotations), and heterodisulfide reductase subunit a (EC 1.8.98.1; 22 annotations). Other frequently exchanged KEGG pathway functions included PST-family polysaccharide transporters (50.5/52.5 normalized annotations were compositionally atypical, representing a 5.3-fold enrichment in the putative HGT pool).

Phylogenetic analysis of HGT revealed similar trends. Genes involved in the KEGG folate biosynthesis pathway are the second most frequently transferred functional class (after unclassified metabolic genes). Methanogenesis genes are also among the most abundant transferred functional classes (rank order 22/173 classes). As in the analysis of genes with atypical dinucleotide compositions, phylogenetic HGT detection found transfer in KO groups involved in methyl-coenzyme M recycling, including those for THMP methyltransferase A, B, and C subunits (EC 2.1.1.86), methyl-coenzyme M reductase system component A2, and heterodisulfide reductase (B and D subunits) (EC 1.8.98.1).

In addition to characterizing KEGG functional categories, ALP gene transfer were analyzed given their proposed importance in M. smithii niche specialization. Because the vast majority of ALP genes could not be assigned to KEGG orthology groups, only a small subset could be tested for gene transfer by using phylogenetic methods. Of the ALPs that could be assigned to KO groups, 6/49 (12.2%) were classified as being horizontally transferred using phylogenetic techniques. When analyzed compositionally, 5 or 6 of 6 of these ALPs were compositionally atypical in dinucleotide use, codon use, and k-words of length 4 or 6.

Remarkably, it was found that in the full pool of 854 ALP OGUs, between 52% and 65% show evidence of transfer across a variety of compositional measures, an enrichment of 6.4- to 9.3-fold when normalized to the overall levels of gene transfer predicted by the same methods. ALPs that could be mapped to KO groups were less compositionally atypical than ALPs as a whole (only 30.6-36.7% were compositionally annotated as transferred for this subgroup). Despite the observation that these genes are highly expressed in M. smithii strains, the ALPs annotated as possessing compositional evidence of transfer do not match the model for ribosomal proteins in their genome, meaning that their expression level alone does not account for their compositional atypicality. Large-scale HGT of ALPs would be consistent with their variability among strains.

TABLE 32
KEGG categories of genes with evidence of horizontal gene transfer

| | Compositionally | | All genes in | | |
|---|---|---|---|---|---|
| | Atypical Genes | | pan-genome | | Fold |
| KEGG Pathway | in pathway* | Percent | in pathway | Percent | Enrichment |
| Unclassified; Poorly | 215 | 12.1 | 3067 | 13.0 | 0.93 |
| Characterized | | | | | |
| Metabolism; Metabolism of | 201 | 11.3 | 2395 | 10.1 | 1.12 |
| Cofactors and Vitamins | | | | | |
| Unclassified; Cellular Processes | 197 | 11.1 | 1031 | 4.4 | 2.54 |
| and Signaling | | | | | |
| Genetic Information Processing; | 187 | 10.5 | 1259 | 5.3 | 1.97 |
| Replication and Repair | | | | | |
| Unclassified; Genetic Information | 143 | 8.0 | 1918 | 8.1 | 0.99 |
| Processing | | | | | |
| Environmental Information | 133 | 7.5 | 1268 | 5.4 | 1.39 |
| Processing; Membrane Transport | | | | | |
| Unclassified; Metabolism | 125 | 7.0 | 1881 | 8.0 | 0.88 |
| Metabolism; Carbohydrate | 75 | 4.2 | 1371 | 5.8 | 0.73 |
| Metabolism | | | | | |
| Metabolism; Nucleotide Metabolism | 70 | 3.9 | 1237 | 5.2 | 0.75 |
| Metabolism; Glycan Biosynthesis | 62 | 3.5 | 298 | 1.3 | 2.74 |
| and Metabolism | | | | | |
| Metabolism; Enzyme Families | 60 | 3.4 | 402 | 1.7 | 1.97 |
| Metabolism; Amino Acid | 58 | 3.3 | 1981 | 8.4 | 0.39 |
| Metabolism | | | | | |
| Metabolism; Energy Metabolism | 57 | 3.2 | 963 | 4.1 | 0.78 |
| Environmental Information | 52 | 2.9 | 78 | 0.3 | 8.89 |
| Processing; Signaling Molecules | | | | | |
| and Interaction | | | | | |
| Genetic Information Processing; | 24 | 1.4 | 384 | 1.6 | 0.84 |
| Folding, Sorting and Degradation | | | | | |
| Metabolism; Xenobiotics | 21 | 1.2 | 516 | 2.2 | 0.53 |
| Biodegradation and Metabolism | | | | | |
| Metabolism; Metabolism of Other | 17 | 1.0 | 269 | 1.1 | 0.85 |
| Amino Acids | | | | | |
| Cellular Processes; Cell Motility | 15 | 0.8 | 57 | 0.2 | 3.50 |
| Human Diseases; Infectious | 11 | 0.6 | 69 | 0.3 | 2.09 |
| Diseases | | | | | |
| Environmental Information | 10 | 0.6 | 119 | 0.5 | 1.12 |
| Processing; Signal Transduction | | | | | |
| Genetic Information Processing; | 9 | 0.5 | 2010 | 8.5 | 0.06 |
| Translation | | | | | |
| Cellular Processes; Transport and | 8 | 0.4 | 34 | 0.1 | 3.09 |
| Catabolism | | | | | |
| Genetic Information Processing; | 8 | 0.4 | 382 | 1.6 | 0.28 |
| Transcription | | | | | |
| Organismal Systems; Immune | 7 | 0.4 | 23 | 0.1 | 4.38 |
| System | | | | | |
| Human Diseases; | 7 | 0.4 | 53 | 0.2 | 1.69 |
| Neurodegenerative Diseases | | | | | |
| Organismal Systems; Excretory | 3 | 0.2 | 21 | 0.1 | 1.77 |
| System | | | | | |
| Metabolism; Biosynthesis of | 2 | 0.1 | 292 | 1.2 | 0.08 |
| Polyketides and Terpenoids | | | | | |
| Organismal Systems; | 1 | 0.1 | 6 | 0.0 | 2.80 |
| Environmental Adaptation | | | | | |
| Organismal Systems; Circulatory | 1 | 0.1 | 3 | 0.0 | 4.80 |
| System | | | | | |
| Metabolism; Lipid Metabolism | 1 | 0.1 | 246 | 1.0 | 0.05 |
| Metabolism; Biosynthesis of Other | 0 | 0.0 | 135 | 0.6 | 0.02 |
| Secondary Metabolites | | | | | |

*Genes shown are atypical in 3-1 dinucleotide usage

TABLE 33
Number of ALPs per M. smithii strain

| | M. smithii strains | Number of ALPs |
|---|---|---|
| MZ twin 1 | METSMITS94A | 52 |
| | METSMITS94B | 57 |
| | METSMITS94C | 52 |
| MZ twin 2 | METSMITS95A | 71 |
| | METSMITS95B | 58 |
| | METSMITS95C | 54 |
| | METSMITS95D | 61 |
| Mother of MZ twins | METSMITS96A | 56 |
| | METSMITS96B | 50 |
| | METSMITS96C | 43 |
| DZ twin 1 | METSMITS145A | 47 |
| | METSMITS145B | 48 |
| DZ twin 2 | METSMITS146A | 44 |
| | METSMITS146B | 41 |
| | METSMITS146C | 89 |
| | METSMITS146D | 43 |
| | METSMITS146E | 52 |
| Mother of DZ twins | METSMITS147A | 51 |
| | METSMITS147B | 53 |
| | METSMITS147C | 53 |
| Culture Collection | METSMIALI (DSM2375) | 31 |
| (previously sequenced) | METSMIF1 (DSM2374) | 34 |
| | MsmPS (NC_009515) | 50 |

### Example 12

### Prospectus

These results lead us to hypothesize that M. smithii strains use their different repertoires of ALPs and the different sensitivities of ALP genes to formate to create diversity in their physical locations and/or their metabolic niches within the gut. Stated another way, these variations in expressed ALP repertoires could have important effects on the ability of different strains to establish syntrophic relationships with bacterial partners that have different abilities to generate formate or other substrates, or that have differing patterns of co-occurrence within an individual over time and between individuals. To further explore this notion, it will be important to define the structures of representative members of different ALP clusters through an M. smithii-directed structural genomics effort: Selection of ALPs could be guided by a number of criteria, including their strain distribution and their patterns of expression, both in vitro in monoculture in the presence of a variety of potential substrates for their metabolic networks, and in vivo in gnotobiotic mice containing various collections of sequenced M. smithii isolates and available cultured co-occurring bacterial taxa. The interactions between isolates and co-occurring bacterial species can also be explored in vitro if cocolonization of gnotobiotic mice proves to be problematic either because of difficulty in identifying suitable host diets or strains that are fit in the mouse gut (e.g., we have not yet been able to achieve persistent colonization of gnotobiotic mice with any of the five strains characterized in vitro by RNA-Seq after inoculating all of them together with a consortium of human gut-derived members of the Firmicutes, Bacteroidetes, and Proteobacteria that include saccharolytic bacteria and hydrogen producers and consumers). A complementary approach will be to select taxa for these in vitro and in vivo studies by predicting potential syntrophic relationships through in silico metabolic reconstructions of the metabolic networks of sequenced co-occurring species and M. smithii isolates, using methods described by Borenstein et al. (47).

### References for Examples 6-12

1. Costello E K, et al. Bacterial community variation in human body habitats across space and time. Science. 2009; 326:1694-1697.
2. Turnbaugh P J, et al. A core gut microbiome in obese and lean twins. Nature. 2009; 457:480-484.
3. Eckburg P B, et al. Diversity of the human intestinal microbial flora. Science. 2005; 308:1635-1638.
4. Dethlefsen L, Huse S, Sogin M L, Relman D A. The pervasive effects of an antibiotic on the human gut microbiota, as revealed by deep 16S rRNA sequencing. PLoS Biol. 2008; 6:e280.
5. Qin J, et al. MetaHIT Consortium A human gut microbial gene catalogue established by metagenomic sequencing. Nature. 2010; 464:59-65.
6. Reyes A, et al. Viruses in the faecal microbiota of monozygotic twins and their mothers. Nature. 2010; 466:334-338.
7. Wolin M J, Miller T L. Interactions of microbial populations in cellulose fermentation. Fed Proc. 1983; 42:109-113.
8. McNeil N I. The contribution of the large intestine to energy supplies in man. Am J Clin Nutr. 1984; 39:338-342.
9. Scanlan P D, Shanahan F, Marchesi J R. Culture-independent analysis of desulfovibrios in the human distal colon of healthy, colorectal cancer and polypectomized individuals. FEMS Microbiol Ecol. 2009; 69:213-221.
10. Bond J H, Jr, Engel R R, Levitt M D. Factors influencing pulmonary methane excretion in man. An indirect method of studying the in situ metabolism of the methane-producing colonic bacteria. J Exp Med. 1971; 133:572-588.
11. Levitt M D, Fume J K, Kuskowski M, Ruddy J. Stability of human methanogenic flora over 35 years and a review of insights obtained from breath methane measurements. Clin Gastroenterol Hepatol. 2006; 4:123-129.
12. Scanlan P D, Shanahan F, Marchesi J R. Human methanogen diversity and incidence in healthy and diseased colonic groups using mcrA gene analysis. BMC Microbiol. 2008; 8:79.
13. Attaluri A, Jackson M, Valestin J, Rao S S C. Methanogenic flora is associated with altered colonic transit but not stool characteristics in constipation without IBS. Am J Gastroenterol. 2010; 105:1407-1411.
14. Pimentel M, et al. Methane, a gas produced by enteric bacteria, slows intestinal transit and augments small intestinal contractile activity. Am J Physiol Gastrointest Liver Physiol. 2006; 290:G1089-G1095.
15. Armougom F, Henry M, Vialettes B, Raccah D, Raoult D. Monitoring bacterial community of human gut microbiota reveals an increase in Lactobacillus in obese patients and methanogens in anorexic patients. PLoS ONE. 2009; 4:e7125.
16. Zhang H, et al. Human gut microbiota in obesity and after gastric bypass. Proc Natl Acad Sci USA. 2009; 106:2365-2370.
17. Florin T H, Zhu G, Kirk K M, Martin N G. Shared and unique environmental factors determine the ecology of methanogens in humans and rats. Am J Gastroenterol. 2000; 95:2872-2879.
18. Pitt P, de Bruijn K M, Beeching M F, Goldberg E, Blendis L M. Studies on breath methane: The effect of ethnic origins and lactulose. Gut. 1980; 21:951-954.
19. Fricke W F, et al. The genome sequence of Methanosphaera stadtmanae reveals why this human intestinal archaeon is restricted to methanol and H2 for methane formation and ATP synthesis. J Bacteriol. 2006; 188:642-658.
20. Hackstein J H P, Van Alen T A, Op Den Camp H, Smits A, Mariman E. Intestinal methanogenesis in primates—a genetic and evolutionary approach. Dtsch Tierarztl Wochenschr. 1995; 102:152-154.
21. Hackstein J H P, et al. Fecal methanogens and vertebrate evolution. Evolution. 1996; 50:559-572.
22. Scholten J C, Culley D E, Brockman F J, Wu G, Zhang W. Evolution of the syntrophic interaction between Desulfovibrio vulgaris and Methanosarcina barkeri: Involvement of an ancient horizontal gene transfer. Biochem Biophys Res Commun. 2007; 352:48-54.
23. Plugge C M, et al. Global transcriptomics analysis of the Desulfovibrio vulgaris change from syntrophic growth with Methanosarcina barkeri to sulfidogenic metabolism. Microbiology. 2010; 156:2746-2756.
24. Friedrich M W. Phylogenetic analysis reveals multiple lateral transfers of adenosine-5′-phosphosulfate reductase genes among sulfate-reducing microorganisms. J Bacteriol. 2002; 184:278-289.
25. Stewart J A, Chadwick V S, Murray A. Carriage, quantification, and predominance of methanogens and sulfate-reducing bacteria in faecal samples. Lett Appl Microbiol. 2006; 43:58-63.
26. Quince C, et al. Accurate determination of microbial diversity from 454 pyrosequencing data. Nat Methods. 2009; 6:639-641.
27. Caporaso J G, et al. QIIME allows analysis of high-throughput community sequencing data. Nat Methods. 2010; 7:335-336.
28. Edgar R C. Search and clustering orders of magnitude faster than BLAST. Bioinformatics. 2010; 26:2460-2461.
29. DeSantis T Z, et al. Greengenes, a chimera-checked 16S rRNA gene database and workbench compatible with ARB. Appl Environ Microbiol. 2006; 72:5069-5072.
30. Ludwig W, et al. ARB: a software environment for sequence data. Nucleic Acids Res. 2004; 32:1363-1371.
31. Cole J R, et al. The Ribosomal Database Project (RDP-II): Sequences and tools for high-throughput rRNA analysis. Nucleic Acids Res. 2005; 33(Database issue):D294-D296.
32. Mackie R I, et al. Ecology of uncultivated Oscillospira species in the rumen of cattle, sheep, and reindeer as assessed by microscopy and molecular approaches. Appl Environ Microbiol. 2003; 69:6808-6815.
33. Yanagita K, et al. Flow cytometric sorting, phylogenetic analysis and in situ detection of Oscillospira guillermondii, a large, morphologically conspicuous but uncultured ruminal bacterium. Int J Syst Evol Microbiol. 2003; 53:1609-1614.
34. Grech-Mora I, et al. Isolation and characterization of Sporobacter termitidis gen nov sp nov, from the digestive tract of the wood-feeding termite Nasutitermes lujae. Int J Syst Bacteriol. 1996; 46:512-518.
35. Drake H L, Gössner A S, Daniel S L. Old acetogens, new light. Ann N Y Acad. Sci. 2008; 1125:100-128.
36. Levitt M D. Volume and composition of human intestinal gas determined by means of an intestinal washout technic. N Engl J Med. 1971; 284:1394-1398.
37. Li Y F, et al. Molecular characterization and hydrogen production of a new species of anaerobe. Environ Sci Health A Tox Hazard Subst Environ Eng. 2005; 40:1929-1938.
38. Ouwerkerk D, Klieve A V, Forster R J, Templeton J M, Maguire A J. Characterization of culturable anaerobic bacteria from the forestomach of an eastern grey kangaroo, Macropus giganteus. Lett Appl Microbiol. 2005; 41:327-333.
39. Kosaka T, et al. The genome of Pelotomaculum thermopropionicum reveals niche-associated evolution in anaerobic microbiota. Genome Res. 2008; 18:442-448.
40. McInerney M J, et al. The genome of Syntrophus aciditrophicus: Life at the thermodynamic limit of microbial growth. Proc Natl Acad Sci USA. 2007; 104:7600-7605.
41. Darling A C, Mau B, Blattner F R, Perna N T. Mauve: Multiple alignment of conserved genomic sequence with rearrangements. Genome Res. 2004; 14:1394-1403.
42. Samuel B S, et al. Genomic and metabolic adaptations of Methanobrevibacter smithii to the human gut. Proc Natl Acad Sci USA. 2007; 104:10643-10648.
43. Giannakis M, et al. Response of gastric epithelial progenitors to Helicobacter pylori Isolates obtained from Swedish patients with chronic atrophic gastritis. J Biol Chem. 2009; 284:30383-30394.
44. Lipinska B, Zylicz M, Georgopoulos C. The HtrA (DegP) protein, essential for Escherichia coli survival at high temperatures, is an endopeptidase. J Bacteriol. 1990; 172:1791-1797.
45. Lee I, Berdis A J, Suzuki C K. Recent developments in the mechanistic enzymology of the ATP-dependent Lon protease from Escherichia coli: Highlights from kinetic studies. Mol Biosyst. 2006; 2:477-483.
46. Lewis A L, et al. Innovations in host and microbial sialic acid biosynthesis revealed by phylogenomic prediction of nonulosonic acid structure. Proc Natl Acad Sci USA. 2009; 106:13552-13557.
47. Borenstein E, Kupiec M, Feldman M W, Ruppin E. Large-scale reconstruction and phylogenetic analysis of metabolic environments. Proc Natl Acad Sci USA. 2008; 105:14482-14487.
48. Zerbino D R, Birney E. Velvet: Algorithms for de novo short read assembly using de Bruijn graphs. Genome Res. 2008; 18:821-829.
49. Darling A C, Mau B, Blattner F R, Perna N T (2004) Mauve: Multiple alignment of conserved genomic sequence with rearrangements. Genome Res 14:1394-1403.
50. Samuel B S, et al. (2007) Genomic and metabolic adaptations of Methanobrevibacter smithii to the human gut. Proc Natl Acad Sci USA 104:10643-10648.
51. Reyes A, et al. (2010) Viruses in the faecal microbiota of monozygotic twins and their mothers. Nature 466:334-338.
52. Fouts D E (2006) Phage_Finder: Automated identification and classification of prophage regions in complete bacterial genome sequences. Nucleic Acids Res 34:5839-5851.
53. Delcher A L, Phillippy A, Carlton J, Salzberg S L (2002) Fast algorithms for large-scale genome alignment and comparison. Nucleic Acids Res 30:2478-2483.
54. Luo Y, Pfister P, Leisinger T, Wasserfallen A (2002) Pseudomurein endoisopeptidases PeiW and PeiP, two moderately related members of a novel family of proteases produced in Methanothermobacter strains. FEMS Microbiol Lett 208:47-51.
55. Karlin S, Mrázek J, Campbell A M (1998) Codon usages in different gene classes of the Escherichia coli genome. Mol Microbiol 29:1341-1355.
56. Zaneveld J R, Nemergut D R, Knight R (2008) Are all horizontal gene transfers created equal? Prospects for mechanism-based studies of HGT patterns. Microbiology 154:1-15.
57. Tsirigos A, Rigoutsos I (2005) A new computational method for the detection of horizontal gene transfer events. Nucleic Acids Res 33:922-933.
58. Lawrence J G, Ochman H (1997) Amelioration of bacterial genomes: Rates of change and exchange. J Mol Evol 44:383-397.
59. Ragan M A, Harlow T J, Beiko R G (2006) Do different surrogate methods detect lateral genetic transfer events of different relative ages? Trends Microbiol 14:4-8.
60. Hsiao W W, et al. (2005) Evidence of a large novel gene pool associated with prokaryotic genomic islands. PLoS Genet 1:e62.
61. Sauer F D (1986) Tetrahydromethanopterin methyltransferase, a component of the methane synthesizing complex of Methanobacterium thermoautotrophicum. Biochem Biophys Res Commun 136:542-547.
62. Hales B A, et al. (1996) Isolation and identification of methanogen-specific DNA from blanket bog peat by PCR amplification and sequence analysis. Appl Environ Microbiol 62:668-675.
63. Eckburg P B, et al. (2005) Diversity of the human intestinal microbial flora. Science 308:1635-1638.
64. DeLong E F (1992) Archaea in coastal marine environments. Proc Natl Acad Sci USA 89:5685-5689.
65. Turnbaugh P J, et al. (2009) A core gut microbiome in obese and lean twins. Nature 457:480-484.
66. Kayar S R, Fahlman A, Lin W C, Whitman W B (2001) Increasing activity of H₂-metabolizing microbes lowers decompression sickness risk in pigs during H₂ dives. J Appl Physiol 91:2713-2719.
67. Knight R, et al. (2007) PyCogent: A toolkit for making sense from sequence. Genome Biol 8:R171.
68. Than C, Ruths D, Nakhleh L (2008) PhyloNet: A software package for analyzing and reconstructing reticulate evolutionary relationships. BMC Bioinformatics 9:322.
69. Edgar R C (2004) MUSCLE: multiple sequence alignment with high accuracy and high throughput. Nucleic Acids Res 32:1792-1797.
70. Price M N, Dehal P S, Arkin A P (2010) FastTree 2—approximately maximum-likelihood trees for large alignments. PLoS ONE 5:e9490.
71. Rey F E, et al. (2010) Dissecting the in vivo metabolic potential of two human gut acetogens. J Biol Chem 285:22082-22090.
72. Ning Z, Cox A J, Mullikin J C (2001) SSAHA: A fast search method for large DNA databases. Genome Res 11:1725-1729.

What is claimed is:

1. An array comprising a substrate, the substrate having disposed thereon at least one nucleic acid, wherein the nucleic acid comprises a nucleic acid sequence selected from the group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, and 95.

2. The array of claim 1, wherein the nucleic acid or nucleic acids are located at a spatially defined address of the array.

3. The array of claim 2, wherein the array has no more than 500 spatially defined addresses.

4. The array of claim 2, wherein the array has at least 500 spatially defined addresses.

5. The array of claim 2, wherein the array further comprises at least one nucleic acid selected from the group consisting of SEQ ID NOs: 97-1240.

6. An array comprising a substrate, the substrate having disposed thereon at least one polypeptide, wherein the polypeptide is encoded by a nucleic acid sequence selected from the nucleic acid sequences selected from the group consisting of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, and 95.

7. The array of claim 5, wherein the polypeptide or polypeptides are located at a spatially defined address of the array.

8. The array of claim 6, wherein the array has no more than 500 spatially defined addresses.

9. The array of claim 6, wherein the array has at least 500 spatially defined addresses.

10. The array of claim 6, wherein the array further comprises at least one polypeptide encoded by a nucleic acid selected from the group consisting of SEQ ID NOs: 97-1240.

11. A method of selecting a compound that has efficacy for modulating a gene product of M. smithii present in the gastrointestinal tract of a subject, the method comprising:
a. comparing a plurality of biomolecules from M. smithii before and after administration of a compound for modulating a gene product of M. smithii, such that if the abundance of a biomolecule that correlates with the gene product is modulated, the compound is efficacious in modulating a gene product of M. smithii; and
b. selecting a compound that modulates a M. smithii gene product,
wherein the gene product correlates with a biomolecule selected from the group consisting of SEQ ID NOs: 1-96.

12. The method of claim 11, wherein the compound inhibits the M. smithii gene product.

13. The method of claim 12, wherein the compound inhibits the growth of M. smithii.

14. The method of claim 12, wherein the compound decreases the efficiency of carbohydrate metabolism in the subject.

15. The method of claim 12, wherein the compound promotes weight loss.

16. The method of claim 11, wherein the compound upregulates the M. smithii gene product.

17. The method of claim 16, wherein the compound promotes the growth of M. smithii.

18. The method of claim 16, wherein the compound increases the efficiency of carbohydrate metabolism in the subject.

19. The method of claim 16, wherein the compound promotes weight gain.
